(19) United States
(12) Patent Application Publication
(10) Pub. No.: US 2022/0064671 A1
(43) Pub. Date: Mar. 3, 2022

(54) METHODS AND SYSTEMS FOR PRODUCING AAV PARTICLES

(71) Applicant: VOYAGER THERAPEUTICS, INC., Cambridge, MA (US)

(72) Inventors: Luis Maranga, Cambridge, MA (US); Christopher J. Morrison, Cambridge, MA (US); Krishanu Mathur, Cambridge, MA (US); Matthew Luther, Cambridge, MA (US); Daniel S. Hurwit, Cambridge, MA (US); Jacob J. Cardinal, Cambridge, MA (US); Lori B. Karpes, Ashland,, MA (US); Aditya Ansondaria, Cambridge, MA (US); James Forster, Cambridge, MA (US); David Dismuke, Cary, NC (US); Robert Steininger, Cambridge, MA (US)

(21) Appl. No.: 17/423,737

(22) PCT Filed: Jan. 17, 2020
(86) PCT No.: PCT/US2020/014000

§ 371 Date: Jul. 16, 2021

Related U.S. Application Data
(60) Provisional application No. 62/931,848, filed on Nov. 7, 2019.
(60) Provisional application No. 62/794,216, filed on Jan. 18, 2019.
(60) Provisional application No. 62/794,204, filed on Jan. 18, 2019.
(60) Provisional application No. 62/794,208, filed on Jan. 18, 2019.
(60) Provisional application No. 62/794,199, filed on Jan. 18, 2019.

(51) Int. Cl. C12N 15/86; A61K 48/00

(52) U.S. Cl. CPC C12N 15/86; C12N 2750/14143; C12N 2750/14151; A61K 48/005

(57) ABSTRACT

The present disclosure describes methods and systems for use in the production of adeno-associated virus (AAV) particles, comprising recombinant adeno-associated virus (rAAV) particles. In certain embodiments, the production process and system use Sf9 insect cells as viral production cells. In certain embodiments, the production process and system use Baculoviral Expression Vectors (BEVs) and Baculoviral Infected Insect Cells (BIICs) in the production of AAV particles.

### CROSS REFERENCE TO RELATED APPLICATIONS

This application claims the benefit of: U.S. Provisional Patent Application No. 62/794,199, filed Jan. 18, 2019, entitled METHODS AND SYSTEMS FOR PRODUCING AAV PARTICLES; U.S. Provisional Patent Application No. 62/794,204, filed Jan. 18, 2019, entitled METHODS AND SYSTEMS FOR PRODUCING AAV PARTICLES; U.S. Provisional Patent Application No. 62/794,208, filed Jan. 18, 2019, entitled METHODS AND SYSTEMS FOR PRODUCING AAV PARTICLES; U.S. Provisional Patent Application No. 62/794,216, filed Jan. 18, 2019, entitled BBC COMPOSITIONS FOR PRODUCING AAV PARTICLES; U.S. Provisional Patent Application No. 62/931,848, filed Nov. 07, 2019, entitled METHODS AND SYSTEMS FOR PRODUCING AAV PARTICLES; the contents of which are each incorporated herein by reference in their entireties.

### REFERENCE TO THE SEQUENCE LISTING

The present application is being filed along with a Sequence Listing in electronic format. The Sequence Listing is provided as a file entitled 20571526PCTSL.txt, created on Jan. 17, 2020, which is 6,478,295 bytes in size. The information in the electronic format of the sequence listing is incorporated herein by reference in its entirety.

### FIELD OF THE DISCLOSURE

The present disclosure describes methods and systems for use in the production of adeno-associated virus (AAV) particles, compositions and formulations, comprising recombinant adeno-associated viruses (rAAV). In certain embodiments, the present disclosure presents methods and systems for designing, producing, clarifying, purifying, formulating, filtering and processing rAAVs and rAAV formulations. In certain embodiments, the production process and system use Spocloptera frugipercla insect cells (such as Sf9 or Sf21) as viral production cells. In certain embodiments, the production process and system use Baculoviral Expression Vectors (BEVs) and/or Baculoviral Infected Insect Cells (BIICs) in the production of rAAVs.

### BACKGROUND

AAVs have emerged as one of the most widely studied and utilized viral vectors for gene transfer to mammalian cells. See, e.g., Tratschin et al., Mol. Cell Biol., 5(11):325-3260 (1985) and Grimm et al., Hum. Gene Ther., 10(15):2445-2450 (1999), the contents of which are each incorporated herein by reference in their entireties insofar as they do not conflict with the present disclosure. Adeno-associated viral (AAV) vectors are promising candidates for therapeutic gene delivery and have proven safe and efficacious in clinical trials. The design and production of improved AAV particles for this purpose is an active field of study.

There remains a need for improved systems and methods for producing AAV capsids proteins, AAV capsids, and corresponding AAV vectors (such as AAV particles).

### SUMMARY

The present disclosure presents methods and systems for producing recombinant adeno-associated viruses (rAAVs).

In certain embodiments, the method for producing a recombinant adeno-associated virus AV) comprises one or more of the following steps: (a) introducing at least one viral production cell (VPC) into a bioreactor and expanding the number of VPCs in the bioreactor to a target VPC cell density, (b) introducing into the bioreactor at least one expression baculovirus infected insect cell (BIIC) which comprises an AAV viral expression construct and at least one payload BIIC which comprises an AAV payload construct; (c) incubating the mixture of VPCs, expression BIICs and payload BIICs in the bioreactor under conditions which result in the production of one or more rAAVs within one or more of the VPCs; (d) harvesting a viral production pool from the bioreactor, wherein the viral production pool comprises a liquid media and the one or more VPCs containing the one or more rAAVs, (e) exposing the one or more VPCs within the viral production pool to chemical lysis using a chemical lysis solution under chemical lysis conditions, wherein the chemical lysis releases the one or more rAAVs from the VPCs into the liquid media of the viral production pool; (f) processing the viral production pool through one or more clarification filtration steps in which the viral production pool is processed through one or more clarification filtration systems; (g) processing the viral production pool through one or more affinity chromatography steps in which the viral production pool is processed through one or more affinity chromatography systems; (h) processing the viral production pool through one or more ion exchange chromatography steps in which the viral production pool is processed through one or more ion exchange chromatography systems; (i) processing the viral production pool through one or more tangential flow filtration (TFF) steps in which the viral production pool is processed through one or more tangential flow filtration (TFF) systems; and (h) processing the viral production pool through one or more virus retentive filtration (VRF) steps in which the viral production pool is processed through one or more virus retentive filtration (VRF) systems.

In certain embodiments, the rAAVs are produced in viral production cells (VPCs) within a bioreactor. In certain embodiments, the volume of the bioreactor is at least 5 L, 10 L, 20 L, 50 L, 100 L, or 200 L. In certain embodiments, the VPCs comprise insect cells. In certain embodiments, the VPCs comprise Sf9 insect cells. In certain embodiments, the rAAVs are produced using a baculovinis production system.

In certain embodiments, the target VPC cell density at BIIC introduction is 2.0-4.0×10⁶ cells/mL, 2.5-3.5×10⁶ cells/mL, or about 3.0-10⁶ cells/mL. In certain embodiments, the ratio of VPC cells at RUC introduction relative to the number of expression BIICs introduced into the bioreactor is between 1:2.0×1.0⁵-1:4.0×1.0⁵ v/v. between 1:2.5×1.0⁵-1:3.5×10⁵ v/v, about 1:2.5×10⁵ v/v, about 1:3.0×10⁵ v/v, about 1:3.5×10⁵ v/v, or about 1:4.0×10⁵ v/v. In certain embodiments, the ratio of VPC cells at BIIC introduction relative to the number of payload BACs introduced into the bioreactor is between 1:5.0×10⁴−2.0×1.0⁵ v/v, between 1:8.0×10⁴-1:1.5×10⁵ v/v, about 1:8.0×10⁴ v/v, about 1:1.0×10⁵ v/v, or about 1:1.5×10⁵ v/v. In certain embodiments, the ratio of expression BACs introduced into the bioreactor relative payload BACs introduced into the bioreactor is between 1:1-5:1, between 2:1-4:1, between 2.5:1-3.5:1, or about 3:1.

In certain embodiments, the method comprises one or more chemical lysis steps in which the viral production pool is exposed to chemical lysis. In certain embodiments, the method comprises: harvesting the viral production pool from the bioreactor, wherein the viral production pool comprises a liquid media and the one or more VPCs containing the one or more rAAVs; and exposing the one or more VPCs within the viral production pool to chemical lysis using a chemical lysis solution under chemical lysis conditions, wherein the chemical lysis releases the one or more rAAVs from the VPCs into the liquid media of the viral production pool. In certain embodiments, the chemical lysis solution comprises a stabilizing additive selected from arginine and salts thereof.

In certain embodiments, the method comprises one or more clarification filtration steps in which the viral production pool is processed through one or more clarification filtration systems. In certain embodiments, the one or more clarification filtration steps comprises processing the viral production pool through a depth filtration system, a 0.2 μm microfiltration system, or a combination thereof In certain embodiments, the one or more clarification filtration steps comprises processing the viral production pool through a depth filtration system and then a 0.2 μm microfiltration system. In certain embodiments, the one or more clarification filtration steps comprises processing the viral production pool through a first depth filtration system, then a second depth filtration system, and then a 0.2 μm microfiltration system.

In certain embodiments, the method comprises one or more affinity chromatography steps in which the viral production pool is processed through one or more affinity chromatography systems. In certain embodiments, the method comprises processing the viral production pool through one or more immunoaffinity chromatography systems in bind-elute mode. In certain embodiments, the immunoaffinity chromatography system comprises one or more recombinant single-chain antibodies which are capable of binding to one or more AAV capsid variants. In certain embodiments, the affinity chromatography system comprises an AVB column resin, AAV9 column resin or AAVX column resin.

In certain embodiments, the method comprises one or more ion exchange chromatography steps in which the viral production pool is processed through one or more ion exchange chromatography systems. In certain embodiments, the method comprises processing the viral production pool through one or more anion exchange chromatography systems in flow-through mode. In certain embodiments, the anion exchange chromatography system comprises a stationary phase which hinds non-viral impurities, non-AAV viral particles, or a combination thereof In certain embodiments, the anion exchange chromatography system comprises a stationary phase which does not bind to the one or more rAAVs in the viral production pool. In certain embodiments, the stationary phase of the anion exchange chromatography system comprises a quaternary amine functional group. In certain embodiments, the anion exchange chromatography system comprises a trimethylammonium ethyl (MME) functional group.

In certain embodiments, the method comprises one or more tangential flow filtration (TFF) steps in which the viral production pool is processed through one or more TFF systems. In certain embodiments, a 50% sucrose mixture is added to the viral production pool prior to the one or more TFT steps. In certain embodiments, the 50% sucrose mixture is added to the viral production pool at a centration between 9-13% v/v prior to the one or more TFF steps. In certain embodiments, the 50% sucrose mixture is added to the viral production pool at a centration between 10-12% v/v prior to the one or more TFF steps. In certain embodiments, the 50% sucrose mixture is added to the viral production pool at a centration of 11% v/v prior to the one or more TFF steps.

In certain embodiments, the one or more TFF steps comprises a first diafiltration step in which at least a portion of the liquid media of the viral production pool is replaced with a low-sucrose diafiltration buffer. In certain embodiments, the low-sucrose diafiltration buffer comprises between 4-6% w/v of a sugar or sugar substitute and between 150-250 mM of an alkali chloride salt. In certain embodiments, the low-sucrose diafiltration buffer comprises between 4.5-5.5% w/v of sucrose and between 210-230 mM sodium chloride. In certain embodiments, the low-sucrose diafiltration buffer comprises 5% w/v of sucrose and 220 mM sodium chloride.

In certain embodiments, the one or more TFF steps comprises an ultrafiltration concentration step, wherein the AAV particles in the viral production pool are concentrated to a target particle concentration. In certain embodiments, the AAV particles in the viral production pool are concentrated to between 1.0×10¹²-5.0×10¹³ vg/mL. In certain embodiments, the AAV particles in the viral production pool are concentrated to between 2.0×10¹² -5.0×10¹² vg/mL. In certain embodiments, the AAV particles in the viral production pool are concentrated to between 1.0×10′³-5.0×10¹³ vg/mL. In certain embodiments, the AAV particles in the viral production pool are concentrated to between 2.0×10¹³-3.0×10¹³ g/mL. In certain embodiments, the AAV particles in the viral production pool are concentrated to 2.7×10¹³ vg/mL.

In certain embodiments, the one or more TFF steps comprises a formulation diafiltration step in which at least a portion of the liquid media of the viral production pool is replaced with a high-sucrose formulation buffer. In certain embodiments, the high-sucrose formulation buffer comprises between 6-8% w/v of a sugar or sugar substitute and between 90-100 mM of an alkali chloride salt. In certain embodiments, the high-sucrose formulation buffer comprises 7% w/v of sucrose and between 90-100 mM sodium chloride. In certain embodiments, the high-sucrose formulation buffer comprises 7% w/v of sucrose, 10 mM Sodium Phosphate, between 95-100 mM sodium chloride, and 0.001% (w/v) Poloxamer 188. In certain embodiments, the formulation diafiltration step is the final diafiltration step in the one or more TFF steps. in certain embodiments, the formulation diafiltration step is the only diafiltration step in the one or more TFF steps.

In certain embodiments, the method comprises one or more virus retentive filtration (VRF) steps in which the viral production pool is processed through one or more VRF systems, In certain embodiments, the VRF system comprises a filter medium which retains particles which are 50 nm or larger. In certain embodiments, the VRF system comprises a filter medium which retains particles which are 35 nm or larger. In certain embodiments, the VRF system comprises a filter medium which retains particles which are 20 nm or larger.

The present disclosure presents methods and systems for producing a pharmaceutical formulation by: (i) providing one or more rAAVs produced by a method or system of the present disclosure; and (ii) combining the one or more rAAVs with one or more one pharmaceutical excipient. The present disclosure presents pharmaceutical formulations produced by a method or system of the present disclosure.

The present disclosure presents methods and systems for producing a gene therapy product by: (i) providing a pharmaceutical formulation comprising rAAVs of the present disclosure, wherein the pharmaceutical formulation and/or rAAVs are produced by a method or system of the present disclosure; and (ii) suitably aliquoting the pharmaceutical formulation into a formulation container.

The present disclosure presents pharmaceutical formulations useful for gene therapy modalities. In certain embodiments, the pharmaceutical formulations comprise rAAVs of the present disclosure. In certain embodiments, the pharmaceutical formulations comprise rAAVs at a concentration less than 5×10¹³ vg/ml. In certain embodiments, the pharmaceutical formulations comprise rAAVs at a concentration between 1.0×10¹²-5.0×10¹³ vg/mL. In certain embodiments, the pharmaceutical formulations comprise rAAVs at a concentration between 1.0×10¹² 5.0×10¹² vg/mL. In certain embodiments, the pharmaceutical formulations comprise rAAVs at a concentration between 1.0×10¹³-5×10¹³ vg/mL. In certain embodiments, the pharmaceutical formulations comprise rAAVs at a concentration of 2.7×10¹³ vg/mL.

### BRIEF DESCRIPTION OF THE FIGURES

The foregoing and other objects, features and advantages will be apparent from the following description of particular embodiments of the present disclosure, as illustrated in the accompanying figures. The figures are not necessarily to scale or comprehensive, with emphasis instead being placed upon illustrating the principles of various embodiments of the present disclosure,

FIG. 1 shows a schematic for one embodiment of a system, and a flow diagram for one embodiment of a process, for producing baculovirus infected insect cells (BIICs) using Viral Production Cells (VPC) and plasmid constructs,

FIG. 2 shows a schematic for one embodiment of a system, and a flow diagram for one embodiment of a process, for producing AAV Particles using Viral Production Cells (VPC) and baculovinis infected insect cells (BIICs).

FIG. 3 shows schematic for one embodiment of a system, and a flow diagram for one embodiment of a process, for producing a Drug Substance by processing, clarifying and purifying a bulk harvest of AAV particles and Viral Production Cells.

FIG. 4A and FIG. 49 show the results of computer modeling for BIIC^{Rep/CaP} cell count (y-axis) vs. BIIC^{ReP/CaP}-to-BIIC^{Payload} v/v ratio (x-axis) in BIIC transfection of viral production cells (VPC). FIG. 4A shows AAV titer (vg/mL) using ddPCR, and FIG. 4B shows Capsid Full %.

FIG. 5A and FIG. 5B show the results of computer modeling for BIIC^{ReP/Cap} cell count (y-axis) vs. VPC cells/mL (x-axis, ×10⁶) in BIIC transfection of viral production cells (VPC). FIG. 5A shows AAV titer (vg/mL) using ddPCR and FIG. 5B shows Capsid

FIG. 6A and FIG. 6B show the results of computer modeling for BIIC^{Rep/Cap}-to-BIIC^{Payload} v/v ratio (y-axis) vs. VPC cells/mL (x-axis, ×10⁶) in BIIC transfection of viral production cells (VPC). FIG. 6A shows AAV titer (vg/mL) using ddPCR, and FIG. 6B shows Capsid Full %.

### DETAILED DESCRIPTION

### I. ADENO-ASSOCIATED VIRUSES (AAVs)

### Overview

Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family comprises the Dependovirus genus which comprises AAV, capable of replication in vertebrate hosts comprising, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in Fields Virology (3d Ed. 1996), the content of which is incorporated herein by reference in its entirety as related to parvoviruses, insofar as it does not conflict with the present disclosure.

AAV have proven to be useful as a biological tool due to their relatively simple structure, their ability to infect a wide range of cells (comprising quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

### AAV Viral Genomes

The wild-type AAV viral genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions comprising, but not limited to, acting as an origin for DNA replication by functioning as primers for the endogenous DNA polymerase complex of the host viral replication cell.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ NO: 123 of U.S. Pat. No. 7,906,111, the content of which is incorporated herein by reference in its entirety as related to AAV9/hu.14, insofar as it does not conflict with the present disclosure) VP1 refers to amino acids 1-736, \/P2 refers to amino acids 138-736, and VP3 refers to amino acids 203-736. In other words, VPT is the full-length capsid sequence, while VP2 and VP3 are shorter components of the whole. As a result, changes in the sequence in the VP3 region, are also changes to VP1 and VP2, however, the percent difference as compared to the parent sequence will be greatest for \/P3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

For use as a biological tool, the wild-type AAV viral genome can be modified to replace the rep/cap sequences with a nucleic acid sequence comprising, a payload region with at least one ITR region. Typically, in recombinant AAV viral genomes there are two ITR regions. The rep/cap sequences can be provided in trans during production to generate AAV particles.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. See Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entireties as related to AAV variants and equivalents, insofar as they do not conflict with the present disclosure.

In certain embodiments, AAV particles, viral genomes and/or payloads of the present disclosure, and the methods of their use, may be as described in WO2017189963, the content of which is incorporated herein by reference in its entirety as related to AAV particles, viral genomes and/or payloads, insofar as it does not conflict with the present disclosure.

AAV particles of the present disclosure may he formulated in any of the gene therapy formulations of the disclosure comprising any variations of such formulations apparent to those skilled in the art. The reference to “AAV particles”, “AAV particle formulations” and “formulated AAV particles” in the present application refers to the AAV particles which may be formulated and those which are formulated without limiting either.

In certain embodiments, AAV particles of the present disclosure are recombinant AAV (rAAV) viral particles which are replication defective, lacking sequences encoding functional Rep and Cap proteins within their viral genome. These defective AAV particles may lack most or all parental coding sequences and essentially carry only one or two AAV ITR sequences and the nucleic acid of interest (i.e. payload) for delivery to a cell, a tissue, an organ or an organism.

In certain embodiments, the viral genome of the AAV particles of the present disclosure comprises at least one control element which provides for the replication, transcription and translation of a coding sequence encoded therein. Not all of the control elements need always be present as long as the coding sequence is capable of being replicated, transcribed and/or translated in an appropriate host cell. Non-limiting examples of expression control elements comprise sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV particles of the present disclosure may be produced recombinantly and may be based on adeno-associated virus (AAV) parent or reference sequences. As used herein, a “vector” is any molecule or moiety which transports, transduces or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV vector genomes contain DNA strands which anneal together to form double stranded. DNA. By skipping second strand synthesis, scAAVs allow for rapid expression in the cell.

In certain embodiments, the AAV viral genome of the present disclosure is a scAAV. In certain embodiments, the AAV viral genome of the present disclosure is a ssAAV.

Methods for producing and/or modifying AAV particles are disclosed in the art, such as pseudotyped AAV particles (KT Patent Publication Nos. WO200028004; WO200123001; WO2004112727; WO 2005005610 and WO 2005072364, the contents of each of which are incorporated herein by reference in their entireties as related to producing and/or modifying AAV particles, insofar as they do not conflict with the present disclosure)

AAV particles may be modified to enhance the efficiency of delivery. Such modified AAV particles can he packaged efficiently and be used to successfully infect the target cells at high frequency and with minimal toxicity. In certain embodiments the capsids of the AAV particles are engineered according to the methods described in US Publication Number US 20130195801, the content of which is incorporated herein by reference in its entirety as related to modifying AAV particles to enhance the efficiency of delivery, insofar as it does not conflict with the present disclosure.

In certain embodiments, the AAV particles comprise a payload region encoding a polypeptide or protein of the present disclosure, and may be introduced into mammalian cells.

### Inverted Terminal Repeats (ITRs)

The AAV particles of the present disclosure comprise a viral genome with at least one ITR region and a payload region. In certain embodiments, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes of the present disclosure may be comprised of naturally occurring polynucleotide sequences or recombinantly derived polynucleotide sequences.

The ITRs may be derived from the same serotype as the capsid, or a derivative thereof. The ITR may be of a different serotype than the capsid. In certain embodiments, the AAV particle has more than one ITR. In a non-limiting example, the AAV particle has a viral genome comprising two ITRs. In certain embodiments, the ITRs are of the same serotype as one another. In another embodiment, the ITRs are of different serotypes. examples comprise zero, one or both of the ITRs having the same serotype as the capsid, In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 130, 140, 141, 142, 145 nucleotides in length, and those having at least 95% identity thereto.

In certain embodiments, each ITR may be 141 nucleotides in length. In certain embodiments, each ITR may be 130 nucleotides in length. In certain embodiments, each ITR may be 119 nucleotides in length.

In certain embodiments, the AAV particles comprise two ITRs and one ITR is 141 nucleotides in length and the other ITR is 130 nucleotides in length. In certain embodiments, the AAV particles comprise two ITRs and both ITRs are 141 nucleotides in length.

Independently, each ITR may be about 75 to about 175 nucleotides in length. The ITR may, independently, have a length such as, but not limited to, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175 nucleotides. The length of the ITR for the viral genome may be 75-80, 75-85, 75-100, 80-85, 80-90, 80-105, 85-90, 85-95, 85-110, 90-95, 90-100, 90-115, 95-100, 95-105, 95-120, 100-105, 100-110, 100-125, 105-110, 105-115, 105-130, 110-115, 110-120, 110-135, 115-120, 115-125, 115-140, 120-125, 120-130, 120-145, 125-130, 125-135, 125-150, 130-135, 130-140, 130-155, 135-140, 135-145, 135-160, 140-145, 140-150, 140-165, 145-150, 145-155, 145-170, 150-155, 150-160, 150-175, 155-160, 155-165, 160-165, 160-170, 165-170, 165-175, and 170-175 nucleotides. As a non-limiting example, the viral genome comprises an ITR that is about 105 nucleotides in length. As a non-limiting example, the viral genome comprises an ITR that is about 141 nucleotides in length. As a non-limiting example, the viral genome comprises an ITR that is about 130 nucleotides in length. As a non-limiting example, the viral genome comprises an ITR that is about 105 nucleotides in length and an ITR that is about 141 nucleotides in length. As a non-limiting example, the viral genome comprises an ITR that is about 105 nucleotides in length and an Ha that is about 130 .nucleotides in length. As a non-limiting example, the viral genome comprises an ITR that is about 130 nucleotides in length and an ITR that is about 141 nucleotides in length. As a non-limiting example, the viral genome may comprise two ITRs, each of which are about 141 nucleotides in length.

### Promoters

In certain embodiments, the payload region of the viral genome comprises at least one clement to enhance the transgene target specificity and expression (See e.g., Powell et al. Viral Expression Cassette Elements to Enhance Transgene Target Specificity and Expression in Gene Therapy, 2015; the content of which is incorporated herein by reference in its entirety as related to payload/transgene enhancer elements, insofar as it does not conflict with the present disclosure). Non-limiting examples of elements to enhance the transgene target specificity and expression comprise promoters, endogenous miRNAs, post-transcriptional regulatory elements (PREs), polyadenylation (PolyA) signal sequences and upstream enhancers (USEs), CMV enhancers and introns.

A person skilled in the art may recognize that expression of the polypeptides of the present disclosure in a target cell may require a specific promoter, comprising but not limited to, a promoter that is species specific, inducible, tissue-specific, or cell cycle-specific (see Parr et al., Nat. Med. 3:1145-9 (1997); the content of which is incorporated herein by reference in its entirety as related to polypeptide expression promoters, insofar as it does not conflict with the present disclosure).

In certain embodiments, the promoter is deemed to be efficient when it drives expression of the polypeptide(s) encoded in the payload region of the viral genome of the AAV particle. In certain embodiments, the promoter is deemed to be efficient when it drives expression in the cell being targeted. In certain embodiments, the promoter has a tropism for the cell being targeted. In certain embodiments, the promoter has a tropism for a viral production cell.

In certain embodiments, the promoter drives expression of the payload for a period of time in targeted cells or tissues. Expression driven by a promoter may be for a period of 1-31 days (or any value or range therein), 1-23 months (or any value or range therein), 2-10 years (or any value or range therein), or more than 10 years. Expression may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years. As a non-limiting example, the promoter can be a weak promoter for sustained expression of a payload in nervous (e.g. CNS) cells or tissues.

In certain embodiments, the promoter drives expression of the polypeptides of the present disclosure for at least 1-11 months (or any individual value therein), 2-65 years any individual value therein), or more than 65 years,

Promoters may be naturally occurring or non-naturally occurring. Non-limiting examples of promoters comprise viral promoters, plant promoters and mammalian promoters. In certain embodiments, the promoters may be human promoters. In certain embodiments, the promoter may be truncated or mutated.

Promoters which drive or promote expression in most tissues comprise, but are not limited to, human elongation factor 1α-subunit (EF1α), cytomegalovirus (CMV) immediate-early enhancer and/or promoter, chicken β-actin (CBA) and its derivative CAG, β glucuronidase (GUSB), or ubiquitin C (UBC). Tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons or subtypes of neurons, astrocytes, or oligodendrocytes.

Non-limiting examples of muscle-specific promoters comprise mammalian muscle creatine kinase (MCK) promoter, mammalian desmin (DES) promoter, mammalian troponin I (TNNI2) promoter, and mammalian skeletal alpha-actin (ASKA) promoter (see, e.g. U.S. Patent Publication US 20110212529, the content of which is incorporated herein by reference in its entirety as related to muscle-specific promoters, insofar as they do no conflict with the present disclosure)

Non-limiting examples of tissue-specific expression elements for neurons comprise neuron-specific enolase (NSE), platelet-derived growth factor (PDGF), platelet-derived growth factor B-chain (PDGF-β), synapsin (Syn), methyl-CpG binding protein 2 (MeCP2), Ca²⁺/calmodulin-dependent protein kinase II (CaMKII), metabotropic glutamate receptor 2 (mGluR2), neurofilament light chain (NFL) or neurofilament heavy chain (NTH), β-globin minigene nβ2, preproenkephalin (PPE), enkephalin (Enk) and excitatory amino acid transporter 2 (EAAT2) promoters. Non-limiting examples of tissue-specific expression elements for astrocytes comprise glial fibrillary acidic protein (GFAP) and EAAT2 promoters. A non-limiting example of a tissue-specific expression element for oligodendrocytes comprises the myelth basic protein (MBP) promoter.

In certain embodiments, the promoter may be less than 1 kb, The promoter may have a length of 200-800 nucleotides (or any value or range therein), or more than 800 nucleotides. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800.

The AAV particles of the present disclosure comprise a viral genome with at least one promoter region. The promoter region(s) may, independently, have a length such as, but not limited to, 4, 5, 6, 7, 8, 9, 10, 11, 12, 13. 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86. 87, 88, 89, 90, 91, 92, 93. 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145,146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212. 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251. 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345. 346, 347, 348, 349, 350, 351, 352, 353, 354, 355, 356, 357, 358, 359, 360, 361, 362, 363. 364, 365, 366, 367, 368, 369, 370, 371, 372, 373, 374, 375, 376, 377, 378, 379, 380, 381, 382, 383, 384, 385, 386. 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492,493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, and 600 nucleotides. The length of the promoter region for the viral genome may be 4-10, 10-20, 10-50, 20-30, 30-40, 40-50, 50-60, 50-100, 60-70, 70-80, 80-90, 90-100, 100-110, 100-150, 110-120, 120-130, 130-140, 140-150, 150-160, 150-200, 160-170, 170-180, 180-190, 190-200, 200-210, 200-250, 210-220, 220-230, 230-240, 240-250, 250-260, 250-300, 260-270, 270-280, 280-290, 290-300, 300-310, 300-350, 310-320, 320-330, 330-340, 340-350, 350-360, 350-400, 360-370, 370-380, 380-390, 390-400, 400-410, 400-450, 410-420, 420-430, 430-440, 440-450, 450-460, 450-500, 460-470, 470-480, 480-490, 490-500, 500-510, 500-550, 510-520, 520-530, 530-540, 540-550, 550-560, 550-600, 560-570, 570-580, 580-590, and 590-600 nucleotides. As a non-limiting example, the viral genome comprises a promoter region that is about 4 nucleotides in length. As a non-limiting example, the viral genome comprises a promoter region that is about 17 nucleotides in length. As a non-limiting example, the viral genome comprises a promoter region that is about 204 nucleotides in length. As a non-limiting example, the viral genome comprises a promoter region that is about 219 nucleotides in length. As a non-limiting example, the viral genome comprises a promoter region that is about 260 nucleotides in length. As a non-limiting example, the viral genome comprises a promoter region that is about 303 nucleotides in length. As a non-limiting example, the viral genome comprises a promoter region that is about 382 nucleotides in length. As a non-limiting example, the viral genome comprises a promoter region that is about 588 nucleotides in length.

In certain embodiments, the promoter may be a combination of two or more components of the same or different starting or parental promoters such as, but not limited to, CMV and CBA. Each component may have a length of 200-800 nucleotides (or any value or range therein), or more than 800 nucleotides. Each component may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. In certain embodiments, the promoter is a combination of a 382 nucleotide CMV-enhancer sequence and a 260 nucleotide CBA-promoter sequence.

In certain embodiments, the viral genome comprises a ubiquitous promoter. Non-limiting examples of ubiquitous promoters comprise CMV, CBA (comprising derivatives CAG, CBh, etc.), EF-1a, PGK, UBC, GUSB (hGBp), and UCOE (promoter of HNRPA2B1-CBX3), In certain embodiments, the promoter region is derived from a CBA promoter sequence. As a non-limiting example, the promoter is 260 nucleotides in length.

Yu et al. (Molecular Pain 2011, 7:63: the content of which is incorporated herein by reference in its entirety, insofar as it does not conflict with the present disclosure) evaluated the expression of eGFP under the CAG, EFIn, PGK and UBC promoters in rat DRG cells and primary DRG cells using lentiviral vectors and found that UBC showed weaker expression than the other 3 promoters and only 10-12% glial expression was seen for all promoters. Soderblom et al. (E. Neuro 2015; the contents of which are each incorporated herein by reference in its entirety) evaluated the expression of eGFP in AAV8 with CMV and UBC promoters and AAV2 with the CMV promoter after injection in the motor cortex. Intranasal administration of a plasmid containing a UBC or EFIa promoter showed a sustained airway expression greater than the expression with the CMV promoter (See e.g., Gill et al., Gene Therapy 2001, Vol. 8, 1539-1546; the content of which is incorporated herein by reference in its entirety, insofar as it does not conflict with the present disclosure) Husain et al. (Gene Therapy 2009; the content of which is incorporated herein by reference in its entirety, insofar as it does not conflict with the present disclosure) evaluated an HβH construct with a hGUSB promoter, a HSV-1LAT promoter and an NSE: promoter and found that the HβH construct showed weaker expression than NSE in mouse brain. Passini and Wolfe (J. Virol. 2001, 12382-12392, the content of which is incorporated herein by reference in its entirety, insofar as it does not conflict with the present disclosure) evaluated the long term effects of the HβH vector following an intraventricular injection in neonatal mice and found that there was sustained expression for at least 1 year. Low expression in all brain regions was found by Xu et al, (Gene Therapy 2001, 8, 1323-1332; the content of which is incorporated herein by reference in its entirety, insofar as it does not conflict with the present disclosure) when NFL and NFH promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), FT, NSF (0.3 kb), GFAP, CMV, hENK, PPE, NFL and NFH. ⁻NFL promoter is a 650-nucleotide promoter and NFH promoter is a 920-nucleotide promoter which are both absent in the liver but NFH promoter is abundant in the sensory proprioceptive neurons, brain and spinal cord and NFH promoter is present in the heart. SCNSA promoter is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. Identification of evolutionary conserved, functional noncoding elements in the promoter region of the sodium channel gene SCN8A, Manim Genome (2007) 18:723-731; and Raymond et al. Expression of Alternatively Spliced Sodium Channel a-subunit genes, Journal of Biological Chemistry (2004) 279(44) 46234-46241; the contents of each of which are incorporated herein by reference in their entireties, insofar as they do not conflict with the present disclosure).

Any of the promoters taught by the aforementioned Yu, Soderblom, Gill, Husain, Passini, Xu, Drews or Raymond may be used in the present disclosures.

In certain embodiments, the promoter is not cell specific.

In certain embodiments, the promoter is a ubiquitin e (HBC) promoter. The UBC promoter may have a size of 300-350 nucleotides. As a non-limiting example, the UBC promoter is 332 nucleotides. In certain embodiments, the promoter is a f3-glucuronidase (GUSB) promoter. The GUSB promoter may have a size of 350-400 nucleotides. As a non-limiting example, the GUSB promoter is 378 nucleotides. In certain embodiments, the promoter is a neurofilament light chain (NFL) promoter. The NFL promoter may have a size of 600-700 nucleotides. As a non-limiting example, the NFL promoter is 650 nucleotides. In certain embodiments, the promoter is a neurofilament heavy chain (NFH) promoter. The NFH promoter may have a size of 900-950 riticleotides. As a non-limiting example, the NFH promoter is 920 nucleotides. In certain embodiments, the promoter is a SCNSA promoter. The SCN8A promoter may have a size of 450-500 nucleotides. As a non-limiting example, the SCN8A promoter is 470 nucleotides.

In certain embodiments, the promoter is a frataxin (FXN) promoter. In certain embodiments, the promoter is a phosphoglycerate kinase 1 (PGK) promoter. In certain embodiments, the promoter is a chicken β-actin (CBA) promoter, or variant thereof. In certain embodiments, the promoter is a CB6 promoter. In certain embodiments, the promoter is a minimal CB promoter. In certain embodiments, the promoter is a cytomegalovirus (CMV) promoter. In certain embodiments, the promoter is a H1 promoter. In certain embodiments, the promoter is a CAG promoter. In certain embodiments, the promoter is a GFAP promoter. In certain embodiments, the promoter is a synapsin promoter. In certain embodiments, the promoter is an engineered promoter. In certain embodiments, the promoter is a liver or a skeletal muscle promoter. Non-limiting examples of liver promoters comprise human α-1-antitrypsin (hAAT) and thyroxine binding globulin (TBG). Non-limiting examples of skeletal muscle promoters comprise Desmin, MCK or synthetic C5-12. In certain embodiments, the promoter is an RNA pot III promoter. As a non-limiting example, the RNA pol III promoter is U6. As a non-limiting example, the RNA pol HI promoter is H1 . In certain embodiments, the promoter is a cardiomyocyte-specific promoter. Non-limiting examples of cardiomyocyte-specific promoters comprise aMHC, cTnT, and CMV-MLC2k. In certain embodiments, the viral genome comprises two promoters. As a non-limiting example, the promoters are an EF1α promoter and a CMV promoter.

In certain embodiments, the viral genome comprises an enhancer element, a promoter and/or a 5′ UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′ UTR/intron may be, but is not limited to, SV40, and CBA-MVM, As a non-limiting example, the enhancer, promoter and/or intron used in combination may be: (1) CMV enhancer, CMV promoter, SV40 5′ UTR intron; (2) CMV enhancer, CBA promoter, SV-40 5′ UTR intron; (3) CMV enhancer, CBA promoter, CBA-MVM 5′ UTR intron; (4) UBC promoter; (5) GUSB promoter; (6) NSE promoter; (7) Synapsin promoter; (8) MeCP2 promoter and (9) GFAP promoter.

In certain embodiments, the viral genome comprises an engineered promoter.

In another embodiment, the viral genome comprises a promoter from a naturally expressed protein.

In certain embodiments, the AAV particles of the present disclosure comprise a viral genome with at least one enhancer region. The enhancer region(s) may, independently, have a length such as, but not limited to, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, and 400 nucleotides. The length of the enhancer region for the viral genome may be 300-310, 300-325, 305-315, 310-320, 315-325, 320-330, 325-335, 325-350, 330-340, 335-345, 340-350, 345-355, 350-360, 350-375, 355-365, 360-370, 365-375, 370-380, 375-385, 375-400, 380-390, 385-395, and 390-400 nucleotides, As a non-limiting example, the viral genome comprises an enhancer region that is about 303 nucleotides in length. As a non-limiting example, the viral genome comprises an enhancer region that is about 382 nucleotides in length.

In certain embodiments, the enhancer region is derived from a CMV enhancer sequence. As a non-limiting example, the CMV enhancer is 382 nucleotides in length.

### Untranslated Regions (UT)

By definition, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the viral genomes of the AAV particles of the present disclosure to enhance expression in hepatic cell lines or liver.

While not wishing to be bound by theory, wild-type 5′ untranslated regions (UTRs) comprise features which play roles in translation initiation. Kozak sequences, which are commonly known to be involved in the process by which the ribosome initiates translation of many genes, are usually comprised in 5′ UTRs. Kozak sequences have the consensus CCR(A/G)CCAUGG, where R is a purine (adenine or guanine) three bases upstream of the start codon (ATG), which is followed by another ‘G’. In certain embodiments, the 5′ UTR in the viral genome comprises a Kozak sequence. In certain embodiments, the 5′ UTR in the viral genome does not comprise a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the content of which is incorporated herein by reference in its entirety as related to AU rich elements, insofar as it does not conflict with the present disclosure): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in viva.

Introduction, removal or modification of 3′ UTR AU rich elements (AREs) can be used to modulate the stability of polynucleotides. When engineering specific polynucleotides, (e.g., payload regions of viral genomes), one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In certain embodiments, the 3′ UTR of the viral genome may comprise an oligo(dT) sequence for templated addition of a poly-A tail.

In certain embodiments, the viral genome may comprise at least one miRNA seed, binding site or full sequence. MicroRNAs (or miRNA or miR) are 19-25 nucleotide noncoding RNAs that bind to the sites of nucleic acid targets and down-regulate gene expression either by reducing nucleic acid molecule stability or by inhibiting translation. A microRNA sequence comprises a “seed” region, i.e., a sequence in the region of positions 2-8 of the mature microRNA., which sequence has perfect Watson-Crick complementarity to the miRNA target sequence of the nucleic acid.

In certain embodiments, the viral genome may be engineered to comprise, alter or remove at least one miRNA binding site, sequence or seed region.

Any UTR from any gene known in the art may he incorporated into the viral genome of the AAV particle. These UTRs, or portions thereof, may be placed in the same orientation as in the gene from which they were selected, or they may be altered in orientation or location. In certain embodiments, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In certain embodiments, the viral genome of the AAV particle comprises at least one artificial UTRs which is not a variant of a wild type UTR.

In certain embodiments, the viral genome of the AAV particle comprises UTRs which have been selected from a family of transcripts whose proteins share a common function, structure, feature or property.

### Polyadenylation Sequence

In certain embodiments, the viral genome of the AAV particles of the present disclosure comprise at least one polyadenylation sequence. The viral genome of the AAV particle may comprise a polyadenylation sequence between the 3′ end of the payload coding sequence and the 5′ end of the 3′ ITR.

In certain embodiments, the polyadenylation sequence or “polyA sequence” may range from absent to about 500 nucleotides in length. The polyadenylation sequence may be, but is not limited to, 1-500 nucleotides in length (or any value or range therein),

In certain embodiments, the polyadenylation sequence is 127 nucleotides in length. In certain embodiments, the polyadenylation sequences is 477 nucleotides in length. In certain embodiments, the polyadenylation sequence is 552 nucleotides in length.

### Linkers

Viral genomes of the present disclosure may be engineered with one or more spacer or linker regions to separate coding or non-coding regions.

In certain embodiments, the payload region of the AAV particle may optionally encode one or more linker sequences. In some cases, the linker may be a peptide linker that may be used to connect the polypeptides encoded by the payload region. Some peptide linkers may be cleaved after expression to separate polypeptide domains, allowing assembly of mature protein fragments. Linker cleavage may be enzymatic. In some cases, linkers comprise an enzymatic cleavage site to facilitate intracellular or extracellular cleavage. Some payload regions encode linkers that interrupt polypeptide synthesis during translation of the linker sequence from an mRNA transcript. Such linkers may facilitate the translation of separate protein domains (e.g., heavy and light chain antibody domains) from a single transcript. In some cases, two or more linkers are encoded by a payload region of the viral genome.

In certain embodiments, payload regions encode linkers comprising furin cleavage sites. Furin is a calcium dependent serine endoprotease that cleaves proteins just downstream of a basic amino acid target sequence (Arg-X-(Arglys)-Arg) (Thomas, G., 2002. Nature Reviews Molecular Cell Biology 3(10): 753-66; the content of which is incorporated herein by reference in its entirety as related to linker molecules or sequences, insofar as it does not conflict with the present disclosure). Furin is enriched in the trans-golgi network where it is involved in processing cellular precursor proteins. Furin also plays a role in activating a number of pathogens. This activity can be taken advantage of for expression of polypeptides of the disclosure.

In certain embodiments, payload regions encode linkers comprising 2A peptides. 2A peptides are small “self-cleaving” peptides (18-22 amino acids) derived from viruses such as foot-and-mouth disease virus (F2A), porcine teschovirus-1 (P2A), Thoseaasigna virus (T2A), or equine rhinitis A virus (E2A). The 2A designation refers specifically to a region of picomavims polyproteins that lead to a ribosomal skip at the glycyl-prolyl bond in the C-terminus of the 2A peptide (Kim, et al.. 2011. PLoS One 6(4): e18556; the content of which is incorporated herein by reference in its entirety as related to 2A peptide linkers, insofar as it does not conflict with the present disclosure). This skip results in a cleavage between the 2A peptide and its immediate downstream peptide. As opposed to IRES linkers, 2A peptides generate stoichiometric expression of proteins flanking the 2A peptide and their shorter length can be advantageous in generating viral expression vectors.

In certain embodiments, payload regions encode linkers comprising IRES. Internal ribosomal entry site (IRES) is a nucleotide sequence (>500 nucleotides) that allows for initiation of translation in the middle of an mRNA sequence (Kim, J. H. et al., 2011. PLoS One 6(4): e18556, the content of which is incorporated herein by reference in its entirety as related to IRES regions and linkers, insofar as it does not conflict with the present disclosure). Use of an IRES sequence ensures co-expression of genes before and after the IRES, though the sequence following the IRES may be transcribed and translated at lower levels than the sequence preceding the IRES sequence.

In certain embodiments, the payload region may encode one or more linkers comprising cathepsin, matrix metalloproteinases or legumain cleavage sites. Such linkers are described e.g. by Cizeau and Macdonald in International Publication No. WO2008052322, the content of which is incorporated herein by reference in its entirety as related to linker molecules and sequences, insofar as it does not conflict with the present disclosure. Cathepsins are a family of proteases with unique mechanisms to cleave specific proteins. Cathepsin B is a cysteine protease and cathepsin D is an aspartyl protease. Matrix metalloproteinases are a family of calcium-dependent and zinc-containing endopeptidases. Legumain is an enzyme catalyzing the hydrolysis of (-Asn-Xaa-) bonds of proteins and small molecule substrates.

In certain embodiments, payload regions may encode linkers that are not cleaved. Such linkers may comprise a simple amino acid sequence, such as a glycine rich sequence. In some cases, linkers may comprise flexible peptide linkers comprising glycine and serine residues. These flexible linkers are small and without side chains so they tend not to influence secondary protein structure while providing a flexible linker between antibody segments (George, R. A., et al., 2002, Protein Engineering 15(11): 871-9; Huston, J. S. et at, 1988. PNAS 85:5879-83; and Shan, D. et al., 1999. Journal of Immunology. 162(11):6589-95; the contents of which are each incorporated herein by reference in their entireties as related to linker molecules and sequences, insofar as they do not conflict with the present disclosure). Furthermore, the polarity of the serine residues improves solubility and prevents aggregation problems.

In certain embodiments, payload regions of the present disclosure may encode small and unbranched serine-rich peptide linkers, such as those described by Huston et al. in U.S. Pat. No. 5,525,491, the content of which is incorporated herein by reference in its entirety as related to linker molecules and sequences, insofar as it does not conflict with the present disclosure. Polypeptides encoded by the payload region of the present disclosure, linked by serine-rich linkers, have increased solubility.

In certain embodiments, payload regions of the present disclosure may encode artificial linkers, such as those described by Whitlow and Filpula in U.S. Pat. No. 5,856,456 and Ladner et al. in U.S. Pat. No. 4,946,778, the contents of which are each incorporated herein by reference in their entireties as related to linker molecules and sequences, insofar as they do not conflict with the present disclosure.

In certain embodiments, the linker region may be 1-50, 1-100, 50-100, 50-150, 100-150, 100-200, 150-200, 150-250, 200-250, 200-300, 250-300, 250-350, 300-350, 300-400, 350-400, 350-450, 400-450, 400-500, 450-500, 450-550, 500-550, 500-600_{;} 550-600, 550-650, or 600-650 nucleotides in length. The linker region may have a length of 1-650 nucleotides (or any value or range therein) r greater than 650. In certain embodiments, the linker region may he 12 nucleotides in length. In certain embodiments, the linker region may be 18 nucleotides in length. In certain embodiments, the linker region may be 45 nucleotides in length. In certain embodiments, the linker region may be 54 nucleotides in length. In certain embodiments, the linker region may be 66 nucleotides in length. In certain embodiments, the linker region may be 75 nucleotides in length. In certain embodiments, the linker region may be 78 nucleotides in length. In certain embodiments, the linker region may be 87 nucleotides in length. In certain embodiments, the linker region may be 108 nucleotides in length. In certain embodiments, the linker region may be 153 nucleotides in length. In certain embodiments, the linker region may be 198 nucleotides in length. In certain embodiments, the linker region may be 623 nucleotides in length.

### Introns and Evans

In certain embodiments, the vector genome comprises at least one element to enhance the transgene target specificity and expression (See e.g., Powell et al. Viral Expression Cassette Elements to Enhance Transgene Target Specificity and Expression in Gene Therapy, 2015; the content of which is incorporated herein by reference in its entirety as related to transgene targeting enhancers, insofar as it does not conflict with the present disclosure) such as an intron. Non-limiting examples of introns comprise, MVM (67-97 bps), F.IX truncated intron 1 (300 bps), β-globin SD/immunoglobulin heavy chain splice acceptor (250 bps), adenovirus splice donor/immunoglobin splice acceptor (500 bps), SV40 late splice donor/splice acceptor (1.9S/16S) (180 bps) and hybrid adenovirus splice donor/IgG splice acceptor (230 bps).

In certain embodiments, the intron or intron portion may be 100-500 nucleotides in length. The intron may have a length of 80, 90, 100, 110, 120, 130, 140, 150, 160, 170, 171, 172, 173, 174, 175, 176, 177, 178, 179, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 or 500. The intron may have a length between 80-100, 80-120, 80-140, 80-160, 80-180, 80-200, 80-250, 80-300, 80-350, 80-400, 80-450, 80-500, 200-300, 200-400, 200-500, 300-400, 300-500, or 400-500.

In certain embodiments, the intron region(s) may, independently, have a length such as, but not limited to, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131 132, 133. 134, 135. 136, 137, 138, 139, 140, 141, 142 143,144, 145, 146, 147, 148, 149, 150, 151, 152, 153, 154, 155, 156, 157, 158, 159, 160 161, 162, 163, 164, 165, 166, 167, 168, 169, 170, 171, 172, 173, 174, 175, 176, 177, 178, 179, 180, 181, 182, 183, 184, 185, 186, 187, 188. 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241. 242, 243, 244, 245, 246, 247, 248, 249, 250, 251, 252, 253, 254, 255, 256, 257, 258, 259. 260, 261, 262, 263, 264, 265, 266, 267, 268. 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, and 350 nucleotides. The length of the intron region for the viral genome may be 25-35, 25-50, 35-45, 45-55, 50-75, 55-65, 65-75, 75-85, 75-100, 85-95, 95-105, 100-125, 105-115, 115-125, 125-135, 125-150, 135-145, 145-155, 150-175, 155-165, 165-175, 175-185, 175-200, 185-195, 195-205, 200-225, 205-215, 215-225, 225-235, 225-250, 235-245, 245-255, 250-275, 255-265, 265-275, 275-285, 275-300, 285-295, 295-305, 300-325, 305-315, 315-325, 325-335, 325-350, and 335-345 nucleotides. As a non-limiting example, the viral genome comprises an intron region that is about 32 nucleotides in length. As a non-limiting example, the viral genome comprises an intron region that is about 172 nucleotides in length. As a non-limiting example, the viral genome comprises an intron region that is about 201 nucleotides in length. As a non-limiting example, the viral genome comprises an intron region that is about 347 nucleotides in length.

In certain embodiments, the intron region is derived from a SV40 intron sequence. As a non-limiting example, the intron is 172 nucleotides in length.

In certain embodiments, the AAV particles of the present disclosure can comprise a viral genome with at least one exon region. The exon region(s) may, independently, have a length such as, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 121, 122, 123 124, 125, 126, 127, 128, 129, 130, 131, 132, 133, 134, 135, 136, 137, 138, 139, 140, 141, 142, 143, 144, 145, 146, 147, 148, 149, and 150 nucleotides. The length of the exon region for the viral genome may be 2-10, 5-10, 5-15, 10-20, 10-30, 10-40, 15-20, 15-25, 20-30, 20-40, 20-50, 25-30, 25-35, 30-40, 30-50, 30-60, 35-40, 35-45, 40-50, 40-60, 40-70, 45-50, 45-55, 50-60, 50-70, 50-80, 55-60, 55-65, 60-70, 60-80, 60-90, 65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 115-120, 115-125, 120-130, 120-140, 120-150, 125-130, 125-135, 130-140, 130-150, 135-140, 135-145, 140-150, and 145-150 nucleotides. As a non-limiting example, the viral genome comprises an exon region that is about 53 nucleotides in length. As a non-limiting example, the viral aenome comprises an exon region that is about 134 nucleotides in length.

### Stuffer Sequences

In certain embodiments, the viral genome comprises at least one element to improve packaging efficiency and expression, such as a sniffer or filler sequence. Non-limiting examples of stutter sequences comprise albumin and/or alpha-1 antitrypsin. Any known viral, mammalian, or plant sequence may be manipulated for use as a stutter sequence.

In certain embodiments, the stutter or filler sequence may be from about 100-3500 nucleotides in length. The stiffer sequence may have a length of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or 3000.

In certain embodiments, the stuffer/filler region(s) may, independently, have a length such as, but not limited to, 50, 51, 52, 53, 54, 55, 56, 57, 58, 59. 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122. 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 2.02, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248. 249, 250, 251, 252, 253, 254, 255, 256, 257, 258. 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295. 296, 297, 298, 299, 300, 301, 302, 303, 304, 305, 306, 307, 308, 309, 310, 311, 312, 313, 314, 315, 316, 317, 318, 319, 320. 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352. 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393. 394, 395, 396, 397, 398 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555. 556, 557, 558, 559, 560, 561, 562, 563, 564, 565, 566, 567, 568, 569, 570, 571, 572. 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618. 619, 620, 621, 622, 623, 624, 625, 626, 627, 628, 629, 630, 631, 632, 633, 634, 635, 636 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698. 699, 700, 701, 702, 703, 704, 705, 706, 707, 708, 709, 710, 711. 712, 713, 714, 715, 716. 717, 718. 719, 720, 721, 722, 723, 724, 725, 726, 727, 728, 729, 730. 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796 797, 798, 799, 800, 801, 802, 803, 804, 805, 806, 807, 808, 809, 810, 811, 812, 813, 814, 815, 816, 817, 818, 819, 820, 821, 822, 823, 824. 825, 826, 827, 828, 829, 830, 831, 832, 833, 834, 835. 836, 837, 838, 839, 840, 841, 842, 843, 844, 845, 846, 847, 848, 849, 850, 851, 852, 853, 854, 855, 856, 857, 858, 859, 860, 861, 862, 863 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120,1121, 1122, 1123, 1124, 1125, 1126, 1127, 1128, 1129, 1130, 1131, 1132., 1133, 1134, 1135, 1136, 1137, 1138, 1139, 1140, 1141, 1142, 1143, 1144, 1145,1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170,1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200. 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240. 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280. 1281, 1282, 1283, 1284, 1285, 1286, 1287, 1288, 1289, 1290. 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420. 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575. 1576, 1577, 1578, 1579, 1580. 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785. 1786, 1787, 1788, 1789, 1790, 1791, 1792, 1793, 1794, 1795, 1796, 1797, 1798, 1799, 1800. 1801, 1802, 1803, 1804, 1805, 1806, 1807, 1808, 1809, 1810,1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840. 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980. 1981, 1982, 1983, 1984, 1985, 1986, 1987, 1988, 1989, 1990, 1991, 1992, 1993, 1994, 1995, 1996, 1997, 1998, 1999, 2000, 2001, 2002, 2003, 2004, 2005, 2006, 2007 2008, 2009, 2010, 2011, 2012, 2013, 2014, 2015, 2016, 2017, 2018, 2019, 2020. 2021, 2022, 2023, 2024, 2025, 2026, 2027, 2028, 2029, 2030, 2031, 2032, 2033, 2034, 2035, 2036, 2037, 2038, 2039, 2040, 2041, 2042, 2043, 2044, 2045, 2046, 2047, 2048, 2049, 2050, 2051, 2052, 2053, 2054, 2055, 2056, 2057, 2058, 2059, 2060, 2061, 2062, 2063, 2064, 2065, 2066, 2067, 2068, 2069, 2070, 2071, 2012, 2073, 2074, 2075, 2076, 2077, 2078, 2079, 2080, 2081, 2082, 2083, 2084, 2085, 2086, 2087, 2088, 2089, 2090, 2091, 2092, 2093, 2094, 2095, 2096, 2097, 2098, 2099, 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830. 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955. 2956, 2957, 2958, 2959, 2960, 2961, 2962, 2963, 2964, 2965, 2966, 2967, 2968, 2969, 2970. 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010. 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, and 3250 nucleotides. The length of any filler region for the viral genome may be 50-100, 100-150, 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1050, 1050-1100, 1100-1150, 1150-1200, 1200-1250, 1250-1300, 1300-1350, 1350-1400, 1400-1450, 1450-1500, 1500-1550, 1550-1600, 1600-1650, 1650-1700, 1700-1750, 1750-1800, 1800-1850, 1850-1900, 1900-1950, 1950-2000, 2000-2050, 2050-2100, 2100-2150, 2150-2200, 2200-2250, 2250-2300, 2300-2350, 2350-2400, 2400-2450, 2450-2500, 2500-2550, 2550 2600, 2600-2650, 2650-2700, 2700-2750, 2750-2800, 2800-2850, 2850-2900, 2900-2950, 2950-3000, 3000-3050, 3050-3100, 3100-3150, 3150-3200, and 3200-3250 nucleotides. As a non-limiting example, the viral genome comprises a filler region that is about 55 nucleotides in length. As a non-limiting example, the viral genome comprises a filler region that is about 56 nucleotides in length. As a non-limiting example, the viral genome comprises a filler region that is about 97 nucleotides in length. As a non-limiting example, the viral genome comprises a filler region that is about 103 nucleotides in length. As a non-limiting example, the viral genome comprises a filler region that is about 105 nucleotides in length. As a non-limiting example, the viral genome comprises a filler region that is about 357 nucleotides in length. As a non-limiting example, the viral genome comprises a filler region that is about 363 nucleotides in length. As a non-limiting example, the viral genome comprises a filler region that is about 712 nucleotides in length. As a non-limiting example, the viral genome comprises a filler region that is about 714 nucleotides in length. As a non-limiting example, the viral genome comprises a filler region that is about 1203 nucleotides in length. As a non-limiting example, the viral genome comprises a filler region that is about 1209 nucleotides in length. As a non-limiting example, the viral genome comprises a filler region that is about 1512 nucleotides in length. As a non-limiting example, the viral genome comprises a filler region that is about 1519 nucleotides in length. As a non-limiting example, the viral genome comprises a filler region that is about 2395 nucleotides in length. As a non-limiting example, the viral genome comprises a filler region that is about 2403 nucleotides in length. As a non-limiting example, the viral genome comprises a filler region that is about 2405 nucleotides in length. As a non-limiting example, the viral genome comprises a filler region that is about 3013 nucleotides in length. As a non-limiting example, the viral genome comprises a filler region that is about 3021 nucleotides in length.

In certain embodiments, the filler region is 714 nucleotides in length.

### Multiple Cloning Site (MCS) Region

In certain embodiments, the AA particles of the present disclosure comprise a viral genome with at least one multiple cloning site (MCS) region. The MCS region(s) may, independently, have a length such as, but not limited to, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121 22, 123, 124, 125, 126, 127, 128, 129, 130, 131, 132, 133, 134, 135, 136, 137, 138, 139, 140, 141, 142, 143, 144, 145, 146, 147, 148, 149, and 150 nucleotides, The length of the MCS region for the viral genome may be 2-10, 5-10, 5-15, 10-20, 10-30, 10-40, 15-20, 15-25, 20-30, 20-40, 20-50, 25-30, 25-35, 30-40, 30-50, 30-60, 35-40, 35-45, 40-50, 40-60, 40-70, 45-50, 45-55, 50-60, 50-70, 50-80, 55-60, 55-65, 60-70, 60-80, 60-90, 65-70, 65-75, 70-80, 70-90, 70-100, 75-80, 75-85, 80-90, 80-100, 80-110, 85-90, 85-95, 90-100, 90-110, 90-120, 95-100, 95-105, 100-110, 100-120, 100-130, 105-110, 105-115, 110-120, 110-130, 110-140, 115-120, 115-125, 120-130, 120-140, 120-150, 125-130, 125-135, 130-140, 130-150, 135-140, 135-145, 140-150, and 145-150 nucleotides, As a non-limiting example, the viral genome comprises an MCS region that is about 5 nucleotides in length. As a non-limiting example, the viral genome comprises an MCS region that is about 10 nucleotides in length. As a non-limiting example, the viral genome comprises an MCS region that is about 14 nucleotides in length. As a non-limiting example, the viral genome comprises an MCS region that is about 18 nucleotides in length. As a non-limiting example, the viral genome comprises an MCS region that is about 73 nucleotides in length. As a non-limiting example, the viral genome comprises an MCS region that is about 121 nucleotides in length.

In certain embodiments, the MCS region is 5 nucleotides in length.

In certain embodiments, the MCS region is 10 nucleotides in length.

### Genome Size

In certain embodiments, the AAV particle which comprises a payload described herein may be single stranded or double stranded vector genome. The size of the vector genome may be small, medium, large or the maximum size. Additionally, the vector genome may comprise a promoter and a polyA tail.

In certain embodiments, the vector genome which comprises a payload described herein may be a small single stranded vector genome. A small single stranded vector genome may be 2.1 to 3.5 kb in size such as about 2.1, 2.2, 2.3, 2.4, 2.5, 2.6, 2.7, 2.8, 2.9, 3.0, 3.1, 3.2, 3.3, 3.4, and 3.5 kb in size. As a non-limiting example, the small single stranded vector genome may be 3.2 kb in size. As another non-limiting example, the small single stranded vector genome may be 2.2 kb in size. Additionally, the vector genome may comprise a promoter and a polyA

In certain embodiments, the vector genome which comprises a payload described herein may be a small double stranded vector genome. A small double stranded vector genome may be 1.3 to 1.7 kb in size such as about 1.3, 1.4, 1.5, 1.6, and 1.7 kb in size. As a. non-limiting example, the small double stranded vector genome may be 1.6 kb in size. Additionally, the vector genome may comprise a promoter and a polyA tail.

In certain embodiments, the vector genome which comprises a payload described herein e.g., polynucleotide, siRNA or dsRNA, may be a medium single stranded vector genome. A medium single stranded vector genome may be 3.6 to 4.3 kb in size such as about 3.6, 3.7, 3.8, 3.9, 4.0, 4.1, 4.2 and 4.3 kb in size. As a non-limiting example, the medium single stranded vector genome may be 4.0 kb in size. Additionally, the vector genome may comprise a promoter and a polyA tail.

In certain embodiments, the vector genome which comprises a payload described herein may be a medium double stranded vector genome. A medium double stranded vector genome may be 1.8 to 2.1 kb in size such as about 1.8, 1.9, 2.0, and 2.1 kb in size. As a non-limiting example, the medium double stranded vector genome may he 2.0 kb in size. Additionally, the vector genome may comprise a promoter and a polyA tail.

In certain embodiments, the vector genome which comprises a payload described herein may be a large single stranded vector genome. A large single stranded vector genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded vector genome may be 4.7 kb in size. As another non-limiting example, the large single stranded vector genome may be 4.8 kb in size. As yet another non-limiting example, the large single stranded vector genome may be 6.0 kb in size. Additionally, the vector genome may comprise a promoter and a polyA tail.

In certain embodiments, the vector genome which comprises a payload described herein may be a large double stranded vector genome. A large double stranded vector genome may be 2.2 to 3.0 kb in size such as about 2.2, 2.3, 2.4, 2.5, 2.6, 2.7, 2.8, 2.9 and 3.0 kb in size. As a non-limiting example, the large double stranded vector genome may be 2.4 kb in size. Additionally, the vector genome may comprise a promoter and a polyA tail. AAV serotypes

AAV particles of the present disclosure may comprise or be derived from any natural or recombinant AAV serotype, According to the present disclosure, the AAV particles may utilize or be based on a serotype or comprise a peptide selected from any of the following: VOY101, VOY201, AAV9, AAV9 K449R, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAV5, AAV6, AAV6.1, AAV6.2, AAV6.1,2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1./hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1 /hu.37, AAV 114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AA.Vhu.44R1, AAVhu.44R2, AAVhu.44R3, AAVhu.45, AAVhu.46, AAVhu.47, AAVhu.48, AAVhu.48R.1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh,46, AAVrh,48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AA.Vrh.64R2, AAVrh.67, AAVrh.73, AAVrh.74, AAVrh.8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T , AAV-PAEC, AAV-LK01, AAV-LK02, AAV-LK03, AAV-LK04, AAV-LK05, AAV-LK06, AAV-LK07, AAV-LK08-AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101 , AAV-8h, AAV-8b, AAV-h, AAV-b, AAV SM 10-2 , AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8 , AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu24, AAV54.1 /hu.21, AAV54.4R/hu.27, AAV46,2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4,AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1,AAV CKd-10,AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4,AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clvh1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV AAV CLv-8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7-AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC 13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, and variants or hybrids/chimeras/combinations thereof.

In certain embodiments, an A.AV serotype used in a composition disclosed herein may be, or comprise, a sequence as described in U.S. Patent Application Publication No. U.S. Pat. No. 20030138772, (the content of which is incorporated herein by reference in its entirety as related to AAV capsids insofar as it does not conflict with the present disclosure), such as, but not limited to, AAV! (SEQ ID NO: 6 and 64 of U.S. Pat. No. 20030138772), AAV2 (SEQ ID NO: 7 and 70 of U.S. Pat. No. 20030138772), AAV3 (SEQ ID NO: 8 and 71 of U.S. Pat. No. 20030138772), AAV4 (SEQ ID NO: 63 of U.S. Pat. No. 20030138772), AAV5 (SEQ ID NO: 114 of U.S. Pat. No. 20030138772), AAV6 (SEQ ID NO: 65 of U.S. Pat. No. 20030138772.), AAV7 (SEQ ID NO: 1-3 of U.S. Pat. No. 20030138772), AAV8 (SEQ ID NO: 4 and 95 of U.S. Pat. No. 20030138772), AAV9 (SEQ ID NO: 5 and 100 of U.S. Pat. No. 20030138772), AAV10 (SEQ ID NO: 117 of U.S. Pat. No. 20030138772), AAV11 (SEQ ID NO: 118 of U.S. Pat. No. 20030138772), AAV12 (SEQ ID NO: 119 of U.S. Pat. No. 200301.38772), AAVrh10 (amino acids 1 to 738 of SEQ ID NO: 81 of U.S. Pat. No. 200301.38772), AAV16.3 (U520030138772 SEQ ID NO: 10), AAV29.3/bb.1 (U.S. Pat. No. 20030138772 SEQ ID NO: 11), AAV29.4 (U.S. Pat. No. 20030138772 SEQ ID NO: 12), AAV29.5/bb.2 (U.S. Pat. No. 20030138772 SEQ ID NO: 13), AAV1.3 (U.S. Pat. No. 20030138772 SEQ ID NO: 14), AAV13.3 (U520030138772 SEQ ID NO: 5), AAV24.1 (U520030138772 SEQ ID NO: 16), AAV27.3 (U.S. Pat. No. 20030138772 SEQ IL) NO: 17)-NAV7.2 (U.S. Pat. No. 20030138772 SEQ ID NO: 18), AAVC1 (U.S. Pat. No. 20030138772 SEQ ID NO: 19), AAVC3 (U.S. Pat. No. 20030138772 SEQ ID NO: 20), AAVC5 (U.S. Pat. No. 20030138772 SEQ ID NO: 21), AAVE1 (U.S. Pat. No. 20030138772 SEQ ID NO: 22), AAVF3 (U.S. Pat. No. 20030138772 SEQ ID NO: 23), AAVF5 (U.S. Pat. No. 20030138772 SEQ ID NO: 24), AAVH6 (U.S. Pat. No. 20030138772 SEQ ID NO: 25). AAVH2 (U520030138772 SEQ ID NO: 26), AAV42-8 (U520030138772 SEQ ID NO: 27), AAV42-15 (U520030138772 SEQ ID NO: 28), AAV42-5b (U.S. Pat. No. 20030138772 SEQ ID NO: 29), AAV42-1b (U.S. Pat. No. 20030138772 SEQ ID NO: 30), AAV42-13 (U.S. Pat. No. 20030138772 SEQ ID NO: 31), AAV42-3a (U.S. Pat. No. 20030138772 SEQ ID NO: 32), AAV42-4 (U.S. Pat. No. 20030138772 SEQ ID NO: 33), AAV42-5a (U520030138772 SEQ ID NO: 34), AAV42-10 (U.S. Pat. No. 20030138772 SEQ ID NO: 35), AAV42-3b (U.S. Pat. No. 20030138772 SEQ ID NO: 36), AAV42-11 (U.S. Pat. No. 20030138772 SEQ ID NO: 37), AAV42-6b (U.S. Pat. No. 20030138772 SEQ NO: 38), AAV43-1 (U.S. Pat. No. 20030138772 SEQ ID NO: 39), AAV43-5 (U.S. Pat. No. 20030138772 SEQ ID NO: 40), AAV43-12 (U.S. Pat. No. 20030138772 SEQ ID NO: 41), AAV43-20 (U.S. Pat. No. 20030138772 SEQ ID NO: 42), AAV43-21 (U.S. Pat. No. 20030138772 SEQ NO: 43), AAV43-23 (U.S. Pat. No. 20030138772 SEQ ID NO: 44), AAV43-25 (U.S. Pat. No. 20030138772 SEQ ID NO: 45), AAV44.1 (U.S. Pat. No. 20030138772 SEQ ID NO: 46), AAV44.5 (U.S. Pat. No. 20030138772 SEQ ID NO: 47), AAV223.1 (U.S. Pat. No. 20030138772 SEQ ID NO: 48), AAV223.2 (U.S. Pat. No. 20030138772 SEQ ID NO: 49), AAV223.4 (U.S. Pat. No. 20030138772 SEQ ID NO: 50), AAV223.5 (U.S. Pat. No. 20030138772 SEQ ID NO: 51), AAV223.6 (U.S. Pat. No. 20030138772 SEQ ID NO: 52), AAV223.7 CU.S. Pat. No. 20030138772 SEQ ID NO: 53), AAVA3.4 (U.S. Pat. No. 20030138772 SEQ ID NO: 54), AAVA3.5 (U.S. Pat. No. 20030138772 SEQ ID NO: 55), AAVA3.7 (U.S. Pat. No. 20030138772 SEQ ID NO: 56), AAVA3.3 (U.S. Pat. No. 20030138772 SEQ NO: 57), AAV42.12 (U.S. Pat. No. 20030138772 SEQ ID NO: 58), AAV44,2 (U.S. Pat. No. 20030138772 SEQ ID NO: 59) AAV42-2 (U.S. Pat. No. 20030138772 SEQ ID NO: 9), or variants or hybridslchimeraslcombinations thereof.

In certain embodiments, the AAV serotype may be, or comprise, a sequence as described in U.S. Patent Application Publication No. U.S. Pat. No. 20150159173 (the content of which is incorporated herein by reference in its entirety as related to AAV capsids, insofar as it does not conflict with the present disclosure), such as, but not limited to. AAV2 (SEQ ID NO: 7 and 23 of U.S. Pat. No. 20150159173), 600 (SEQ ID NO: 1 of U.S. Pat. No. 201501.59173), 1702/33 (SEQ NO: 2 of U.S. Pat. No. 20150159173), rh39 (SEQ ID NO: 3, 20 and 36 of U.S. Pat. No. 201501.59173), rh46 (SEQ ID NO: 4 and 22 of U.S. Pat. No. 20150159173), rh73 (SEQ ID NO: 5 of U.S. Pat. No. 20150159173), rh74 (SEQ ID NO: 6 of U.S. Pat. No. 20150159173), AAV6.1 (SEQ ID NO: 29 of U.S. Pat. No. 20150159173), rh.8 (SEQ ID NO: 41 of U.S. Pat. No. 20150159173), rh.48.1 (SEQ ID NO: 44 of U.S. Pat. No. 201.50159173), hu.44 (SEQ ID NO: 45 of U.S. Pat. No. 20150159173), hu.29 (SEQ ID NO: 42 of U.S. Pat. No. 20150159173), hu.48 (SEQ ID NO: 38 of U.S. Pat. No. 20150159173), rh54 (SEQ ID NO: 49 of U.S. Pat. No. 20150159173), AAV2 (SEQ ID NO: 7 of U.S. Pat. No. 20150159173), cy.5 (SEQ ID NO: 8 and 24 of U.S. Pat. No. 20150159173), rh.10 (SEQ ID NO: 9 and 25 of U.S. Pat. No. 20150159173), rh.13 (SEQ ID NO: 10 and 26 of U.S. Pat. No. 201.50159173), AAV1 (SEQ ID NO: 11 and 27 of U.S. Pat. No. 20150159173), AAV3 (SEQ ID NO: 12 and 28 of U.S. Pat. No. 20150159173), AAV6 (SEQ ID NO: 13 and 29 of U.S. Pat. No. 20150159173), AAV7 (SEQ ID NO: 14 and 30 of U.S. Pat. No. 20150159173), AAV8 (SEQ ID NO: 15 and 31 of U.S. Pat. No. 20150159173), hu.13 (SEQ ID NO: 16 and 32 of U.S. Pat. No. 20150159173), hu.26 (SEQ ID NO: 17 and 33 of U.S. Pat. No. 20150159173), hu.37 (SEQ ID NO: 18 and 34 of U.S. Pat. No. 20150159173), hu.53 (SEQ ID NO: 19 and 35 of U.S. Pat. No. 20150159173), rh.43 (SEQ ID NO: 21 and 37 of U.S. Pat. No. 20150159173), rh2 (SEQ ID NO: 39 of U.S. Pat. No. 201501591.73), rh.37 (SEQ ID NO: 40 of U.S. Pat. No. 201501591.73), rh.64 (SEQ ID NO: 43 of U.S. Pat. No. 201501591.73), rh.48 (SEQ ID NO: 44 of U.S. Pat. No. 20150159173), ch.5 (SEQ ID NO 46 of U.S. Pat. No. 20150159173)- rh.67 (SEQ ID NO: 47 of U.S. Pat. No. 20150159173), rh.58 (SEQ ID NO: 48 of U.S. Pat. No. 20150159173), or variants thereof comprising, but not limited to Cy5R1, Cy5R2, Cy5R3, Cy5R4, rh,13R, rh.37R2, rh,2R, rh.8R, 1711.48.1, 171148.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, or hu.48R3, or a variant or hybrid/chimera/combination thereof.

In certain embodiments, the AAV serotype may be, or comprise, a sequence as described in U.S. Pat. No. 7,198,951 (the content of which is incorporated herein by reference in its entirety as related to AAV capsids, insofar as it does not conflict with the present disclosure), such as, but not limited to, AAV9 (SEQ ID NO: 1-3 of U.S. Pat. No. 7,198,951), AAV2 (SEQ ID NO: 4 of U.S. Pat. No. 7,198,951), AAV1 (SEQ ID NO: 5 of U.S. Pat. No. 7,198,951), AAV3 (SEQ ID NO: 6 of U.S. Pat. No. 7,198,951), or AAV8 (SEQ ID NO: 7 of U.S. Pat. No. 7,198,951), or a variant or hybrid/chimera or combination thereof.

In certain embodiments, the AAV serotype may be the AAV9 sequence as described by N Pulicherla et al. (Molecular Therapy 19(6):1070-1078 (2011) (the content of which is incorporated herein by reference in its entirety as related to AAV capsids, insofar as it does not conflict with the present disclosure), or may he a variant thereof, such as but not limited to, AAV9.9, AAV9.11, AA.V9.13, AAV9.16, AAV9.24, AAV9.45, -NAV9.45, AAV9.47, AAV9.61, AAV9.68, or AAV9.84.

In certain embodiments, the AAV serotype may he, or comprise, a sequence as described in U.S. Pat. No. 6,156,303 (the content of which is incorporated herein by reference in its entirety as related to AAV capsids, insofar as it does not conflict with the present disclosure), such as, but not limited to, AAV3B (SEQ ID NO: 1 and 10 of U.S. Pat. No. 6,156,303), AAV6 (SEQ ID NO: 2, 7 and 11 of U.S. Pat. No. 6,156,303), AAV2 (SEQ ID NO: 3 and 8 of U.S. Pat. No. 6,156,303), AAV3A (SEQ ID NO: 4 and 9, of U.S. Pat. No. 6,156,303), or a derivative or a variant or hybrid/chimera or combination thereof.

In certain embodiments, the AAV serotype may be, or comprise, a sequence as described in U.S. Patent Application Publication No. U.S. Pat. No. 20140359799 (the content of which is incorporated herein by reference in its entirety as related to AAV capsids, insofar as it does not conflict with the present disclosure), such as, but not limited to, AAV8 (SEQ ID NO: 1 of U.S. Pat. No. 20140359799), AAVDJ (SEQ ID NO: 2 and 3 of U.S. Pat. No. 20140359799), or variants thereof.

In certain embodiments, the serotype may be AAVDJ or a variant thereof, such as AAVDJ8 (or AAV-DJ8), as described by Grimm et al. (Journal of Virology 82(12): 5887-5911 (2008), the content of which is incorporated herein by reference in its entirety as related to AAV capsids, insofar as it does not conflict with the present disclosure). The amino acid sequence of AAVDJ8 may comprise two or more mutations effective to remove the heparin binding domain (I-IBD). As a non-limiting example, the AAV-DJ sequence described as SEQ ID NO: 1 in U.S. Pat. No. 7,588,772 (the content of which is incorporated herein by reference in its entirety as related to AAV capsids, insofar as it does not conflict with the present disclosure), may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, the AAV-DJ sequence described in U.S. Pat. No. 7,588,772 may comprise three mutations: (1) K406R where lysine (K; Lys) at amino acid 406 is changed to arginine (R; Arg), (2) R587Q where arginine (R; Arg) at amino acid 587 is changed to glutamine (Q; Gin) and (3) R590T where arginine (R; Arg) at amino acid 590 is changed to threonine (T; Thr).

In certain embodiments, the AAV serotype may be, or comprise, a sequence of AAV4 as described in International Publication No. WO1998011244 (the content of which is incorporated herein by reference in its entirety as related to AAV capsids, insofar as it does not conflict with the present disclosure), such as, but not limited to AAV4 (SEQ ID NO: 1-20 of WO1998011244).

In certain embodiments, the AAV serotype may be, or comprise, a mutation in the AAV2 sequence to generate AAV2G9 as described in International Publication No. WO2014144229 (the content of which is incorporated herein by reference in its entirety as related to AAV capsids, insofar as it does not conflict with the present disclosure)

In certain embodiments, the AAV serotype may be, or comprise, a sequence as described in International Publication No. WO2005033321 (the content of which is incorporated herein by reference in its entirety as related to AAV capsids, insofar as it does not conflict with the present disclosure), such as, but not limited to AAV3-3 (SEQ ID NO: 217 of WO200503332 AAV 1 (SEQ ID NO: 219 and 202 of WO2005033321), AAV106.1,cliu.37 (SEQ ID No: 10 of WO2005033321), AAV114.3/hu.40 (SEQ ID No: 11 of WO2005033321), AAV127.2/hu.41 (SEQ ID NO:6 and 8 of WO2005033321), AAV128.3/hu.44 (SEQ ID No: 81 of WO2005033321), AAV130.4/hu.48 (SEQ ID NO: 78 of WO2005033321), AAV145.1/hu.53 (SEQ ID No: 176 and 177 of WO2005033321), AAV145.6/hu.56 (SEQ ID NO: 168 and 192 of WO2005033321), AAV16.12/hu.11 (SEQ ID NO: 153 and 57 of WO2005033321), AAV1.6.8/hu.10 (SEQ m NO: 156 and 56 of WO2005033321), AAV161.10/hu.60 (SEQ ID No: 170 of WO2005033321), AAV161.6/hu.61 (SEQ ID No: 174 of WO2005033321), AAV1-7/rh.48 (SEQ ID NO: 32 of WO2005033321), AAV1-8/rh.49 (SEQ ID NOs: 103 and 25 of WO2005033321)-NAV2 (SEQ ID NO: 211 and 221 of WO2005033321), AAV2-15/rh.62 (SEQ ID No: 33 and 114 of WO2005033321), AAV2-3/rh.61 (SEQ ID NO: 21 of WO2005033321), AAV2-4/rh.50 (SEQ ID No: 23 and 108 of WO2005033321), AAV2-5/rh.51 (SEQ ID NO: 104 and 22 of WO2005033321), AAV3.1./hu.6 (SEQ ID NO: 5 and 84 of WO2005033321), AAV3.1/hu.9 (SEQ ID NO: 155 and 58 of WO2005033321), AAV3-11/rh.53 (SEQ ID NO: 186 and 176 of WO2005033321), AAV3-3 (SEQ ID NO: 200 of WO2005033321), AAV33.12/hu.17 (SEQ ID NO:4 of WO2005033321), AAV33.4/hu.15 (SEQ ID No: 50 of WO2005033321), AAV33.8/hu.16 (SEQ ID No: 51 of WO2005033321), AAV3-9/rh.52 (SEQ ID NO: 96 and 18 of WO2005033321), AAV4-19/rh.55 (SEQ ID NO: 117 of WO2005033321), AAV4-4 (SEQ ID NO: 201 and 218 of WO2005033321), AAV4-9/rh.54 (SEQ ID NO: 116 of WO2005033321), AAV5 (SEQ ID NO: 199 and 216 of WO2005033321), AAV52.1/hu.20 (SEQ ID NO: 63 of WO2005033321), AAV52/hu.19 (SEQ ID NO: 133 of WO2005033321), AAV5-22/rh.58 (SEQ m No: 27 of WO2005033321), AAV5-3/rh57 (SEQ ID NO: 105 of WO2005033321), AV5-3/rh.57 (SEQ ID No: 26 of WO2005033321), AAV58.2thu.25 (SEQ ID No: 49 of WO2005033321), AAV6 (SEQ ID NO: 203 and 220 of WO2005033321, AAV7 (SEQ ID NO: 222 and 213 of WO2005033321), AAV7.3/hu.7 (SEQ ID No: 55 of WO2005033321), AAV8 (SEQ ID NO: 223 and 214 of WO2005033321), AAVH-1/hu.1 (SEQ ID No: 46 of WO2005033321), AAVH-5/hu.3 (SEQ ID No: 44 of WO2005033321), AAVhu.1 (SEQ ID NO: 144 of WO2005033321), AA Vhu.10 (SEQ ID NO: 156 of WO2005033321), AAVhu.11 (SEQ ID NO: 153 of WO2005033321), AAVhu.12 (WO2005033321 SEQ ID NO: 59), AAVhu.13 (SEQ ID NO: 129 of WO2005033321), AAVhu.14/AAV9 (SEQ ID NO: 123 and 3 of WO2005033321), AAVhu.15 (SEQ ID NO: 147 of WO2005033321), AAVhu.16 (SEQ ID NO: 148 of WO2005033321), AAVhu.17 (SEQ ID NO: 83 of WO2005033321), AAVhu.18 (SEQ ID NO: 149 of WG2005033321), AAVhu.19 (SEQ ID NO: 133 of WO2005033321), AAVhu.2 (SEQ ID NO: 143 of WO2005033321), AAVhu.20 (SEQ ID NO: 134 of WO2005033321), AAVhu.21 (SEQ ID NO: 135 of WO2005033321), AAVhu.22 (SEQ ID NO: 138 of WO2005033321), AAVhu.23.2 (SEQ ID NO: 137 of WO2005033321), AAVhu.24 (SEQ ID NO: 136 of WO2005033321), AAVhu.25 (SEQ ID NO: 146 of WO2005033321), AAVhu.27 (SEQ ID NO: 140 of WO2005033321), AAVhu.29 (SEQ IL) NO: 132 of WO2005033321), AAVhu.3 (SEQ ID NO: 145 of WO2005033321), AAVhu.31 (SEQ ID NO: 121 of WO2005033321), AAVhu.32 (SEQ ID NO: 122 of WO2005033321), AAVhu.34 (SEQ ID NO: 125 of WO2005033321), AAVhu.35 (SEQ IL) NO: 164 of WO2005033321), AAVhu.37 (SEQ ID NO: 88 of WO2005033321), AAVhu.39 (SEQ ID NO: 102 of WO2005033321), AAVhu.4 (SEQ ID NO: 141 of WO2005033321), AAVhu.40 (SEQ ID NO: 87 of WO2005033321), AAVhu.41 (SEQ ID NO: 91 of WO2005033321), AAVhu.42 (SEQ ID NO: 85 of WO2005033321), AAVhu.43 (SEQ ID NO: 160 of WO2005033321), AAVhu.44 (SEQ NO: 144 of WO2005033321), AAVhu.45 (SEQ ID NO: 127 of WO2005033321), AAVhu.46 (SEQ ID NO: 159 of WO2005033321), AAVhu.47 (SEQ ID NO: 128 of WO2005033321), AAVhu.48 (SEQ ID NO: 157 of WO2005033321), AAVhu.49 (SEQ NO: 189 of WO2005033321), AAVhu.51 (SEQ ID NO: 190 of WO2005033321), AAVhu.52 (SEQ ID NO: 191 of WO2005033321), AAVhu.53 (SEQ ID NO: 186 of WO2005033321), AAVhu.54 (SEQ ID NO: 188 of WO2005033321), AAVhu.55 (SEQ ID NO: 187 of WO2005033321), AAVhu.56 (SEQ ID NO: 192 of WO2005033321), AAVhu.57 (SEQ ID NO: 193 of WO2005033321), AAVhu.58 (SEQ ID NO: 194 of WO2005033321), AAVhu.6 (SEQ ID NO: 84 of WO2005033321), AAVhu.60 (SEQ ID NO: 184 of WO2005033321), AAVhu.61 (SEQ ID NO: 185 of WO2005033321), AAVhu.63 (SEQ ID NO: 195 of WO2005033321), AAVhu.64 (SEQ ID NO: 196 of WO2005033321), AAVhu.66 (SEQ ID NO: 197 of WO2005033321), AAVhu.67 (SEQ ID NO: 198 of WO2005033321), AAVhu.7 (SEQ m NO: 150 of WO2005033321), AAVhu.8 (WO2005033321 SEQ ID NO: 12), AAVhu.9 (SEQ ID NO: 155 of WO2005033321), AAVLG-10/rh.40 (SEQ ID No: 14 of WO2005033321), AAVLG-4/rh.38 (SEQ ID NO: 86 of WO2005033321), AAVLG-4/rh.38 (SEQ ID No: 7 of WO2005033321), AAVN721-8/rh,43 (SEQ ID NO: 163 of WO2005033321), AAVN721-8/rh.43 (SEQ ID No: 43 of WO2005033321), AAVpi.1 (WO2005033321 SEQ ID NO: 28), AAVpi.2 (WO2005033321 SEQ ID NO: 30), AAVpi.3 (WO2005033321 SEQ ID NO: 29), AAVrh.38 (SEQ ID NO: 86 of WO2005033321), AAVrh.40 (SEQ ID NO: 92 of WO2005033321), AAVrh.43 (SEQ ID NO: 163 of WO2005033321), AAVrh.44 (WO2005033321 SEQ ID NO: 34), AAVrh.45 (WO2005033321 SEQ ID NO: 41), AAVrh.47 (WO2005033321 SEQ NO: 38), AAVrh.48 (SEQ NO: 115 of WO2005033321), AAVrh.49 (SEQ ID NO: 103 of WO2005033321), AAVrh.50 (SEQ ID NO: 108 of WO2005033321), AAVrh.51 (SEQ ID NO: 104 of WO2005033321), AAVrh.52 (SEQ ID NO: 96 of WO2005033321), AAA/rh53 (SEQ ID NO: 97 of WO2005033321), AAVrh.55 (WO2005033321 SEQ ID NO: 37), AAVrh.56 (SEQ ID NO: 152 of WO2005033321), AAVrh.57 (SEQ ID NO: 105 of WO2005033321), AAVrh.58 (SEQ ID NO: 106 of WO2005033321), AAVrh.59 (WO2005033321 SEQ ID NO: 42), AAVrh.60 (WO2005033321 SEQ ID NO: 31)-NAVrh.61 (SEQ ID NO: 107 of WO2005033321), AAVrh.62 (SEQ m NO: 114 of WO2005033321), AAVrh.64 (SEQ ID NO: 99 of WO2005033321), AAVrh.65 (WO2005033321 SEQ ID NO: 35), AAVrh.68 (WO2005033321 SEQ ID NO: 16), AAVrh.69 (WO2005033321 SEQ ID NO: 39), AAVrh.70 (WO2005033321 SEQ ID NO: 20), AAVrh.72 (WO2005033321 SEQ ID NO: 9), or variants thereof comprising, but not limited to, AAVcy.2, AAVcy.3, AAVcy.4, AAVcy.5, AAVcy.6, AAVrh.12, AAVrh.17, AAVrh.18, AAVrh.19, AAVrh.21, AAVrh.22, AAVrh.23, AAVrh.24, AAVrh.25. AAVrh.25/42 15, AAVrh.31, AAVrh.32, AAVrh.33, AAVrh.34, AAVrh.35, AAVrh.36, AAVrh.37, or AAVrh14 (the contents of which are each incorporated herein by reference in their entireties as related to AAV capsids, insofar as they do not conflict with the present disclosure). Non limiting examples of variants comprise SEQ ID NO: 13, 15, 17, 19, 24, 36, 40, 45, 47, 48, 51, 52, 53, 54, 60, 61, 62, 64, 65, 66, 67, 68, 69 70, 71, 72, 73, 74, 75, 76, 77, 79, 80. 82, 89, 90, 93, 94, 95, 98. 100, 101, 109,110, 111, 112, 113, 118, 119, 120, 124, 126, 131, 139, 142, 151, 154, 158, 161, 162, 165, 166, 167, 168, 169, 170, 171, 172, 173, 174, 175, 176, 177, 178, 179, 180, 181, 182, 183, 202, 204, 205, 206, 207, 208, 209, 210, 211, 212, 215, 219, 224, 225, 226, 227, 228, 229, 230, 231, 232, 233, 234, 235 or 236 of WO2005033321 (the content of which is incorporated herein by reference in its entirety as related to AAV capsids, insofar as it does not conflict with the present disclosure).

In certain embodiments, the AAV serotype may be, or comprise, a sequence as described in International Publication No. WO2015168666 (the content of which is incorporated herein by reference in its entirety as related to AAV capsids, insofar as it does not conflict with the present disclosure), such as, but not limited to, AAVrh8R (SEQ ID NO: 9 of WO2015168666), AAVrh8R A586R mutant (SEQ ID NO: 10 of WO2015168666), AAVrh8R R533A mutant (SEQ ID NO: 11 of WO2015168666), or variants thereof

In certain embodiments, the AAV serotype may be, or comprise, a sequence as described in U.S. Pat. No. 9,233,131 (the content of which is incorporated herein by reference in its entirety as related to AAV capsids, insofar as it does not conflict with the present disclosure), such as, but not limited to, AAVhE1.1 (SEQ ID NO:44 of U.S. Pat. No. 9,233,131), AAVhEr1.5 (SEQ ID NO:45 of U.S. Pat. No. 9,233,131), AAVhER1.14 (SEQ ID NO:46 of U.S. Pat. No. 9,233,131), AAVhEr1.8 (SEQ NO:47 of U.S. Pat. No. 9,233,131), AAVhEr1.16 (SEQ NO:48 of U.S. Pat. No. 9,233,131), AAVhEr1.18 (SEQ ID NO:49 of U.S. Pat. No. 9,233,131), AAVhEr1.35 (SEQ ID NO:50 of U.S. Pat. No. 5,923,3131), AAVhEr1.7 (SEQ ID NO:51 of U.S. Pat. No. 9,233,131), AAVhEr1.36 (SEQ IL) NO:52 of U.S. Pat. No. 9,233,131), AAVhEr2.29 (SEQ ID NO:53 of U.S. Pat. No. 9,233,131), AAVhEr2.4 (SEQ ID NO:54 of U.S. Pat. No. 9,233,131), AAVhEr2.16 (SEQ ID NO:55 of U.S. Pat. No. 9,233,131), AAVhEr2.30 (SEQ ID NO:56 of U.S. Pat. No. 9,233,131), AAVhEr2.31 (SEQ ID NO:58 of U.S. Pat. No. 9,233,131), AAVhEr2.36 (SEQ ID NO:57 of U.S. Pat. No. 9,233,131), AAVhER1.23 (SEQ ID No. 53 of U.S. Pat. No. 9233131), AAVhEr3.1 (SEQ NO:59 of U.S. Pat. No. 9,233,131), AAV2.5T (SEQ ID NO:42 of U.S. Pat. No. 9,233,131), or variants thereof.

In certain embodiments, the AAV serotype may be, or comprise, a sequence as described in U.S. Patent Application Publication No. U.S. Pat. No. 20150376607 (the content of which is incorporated herein by reference in its entirety as related to AAV capsids, insofar as it does not conflict with the present disclosure), such as, but not limited to, AAV-PAEC (SEQ ID NO:1 of U.S. Pat. No. 20150376607), AAV-LK01 (SEQ IL) NO:2 of U.S. Pat. No. 20150376607), AAV-LK02 (SEQ NO:3 of U.S. Pat. No. 20150376607)-NAV-LK03 (SEQ NO:4 of U.S. Pat. No. 20150376607), (SEQ ID NO:5 of U.S. Pat. No. 20150376607), AAV-LK05 (SEQ ID NO:6 of US 20150376607), AAV-LK06 (SEQ ID NO:7 of U.S. Pat. No. 20150376607), AAV-LK07 (SEQ ID NO:8 of U.S. Pat. No. 20150376607), AAV-LK08 (SEQ ID NO:9 of U.S. Pat. No. 20150376607), AAV-LK09 (SEQ ID NO:10 of U.S. Pat. No. 20150376607), AAV-LK10 (SEQ ID NO:11 of U.S. Pat. No. 20150376607), AAV-LK11 (SEQ ID NO:12 of U.S. Pat. No. 20150376607), AAV-LK12 (SEQ ID NO:13 of U.S. Pat. No. 20150376607), AAV-LK13 (SEQ ID NO:14 of U.S. Pat. No. 20150376607), AAV-LK14 (SEQ ID NO:15 of U.S. Pat. No. 20150376607), AAV-LK15 (SEQ ID NO:16 of U.S. Pat. No. 20150376607), AAV-LK16 (SEQ ID NO:17 of U.S. Pat. No. 20150376607), AAV-LK17 (SEQ NO:18 of U.S. Pat. No. 20150376607), AAV-LK18 (SEQ ID NO:19 of U.S. Pat. No. 20150376607), AAV-LK19 (SEQ ID NO:20 of U.S. Pat. No. 20150376607), AAV-PAEC2 (SEQ ID NO:21 of U.S. Pat. No. 20150376607), AAV-PAEC4 (SEQ ID NO:22 of U.S. Pat. No. 20150376607), AAV-PAEC6 (SEQ ID NO: 23 of U.S. Pat. No. 20150376607), AAV-PAEC7 (SEQ ID NO:24 of U520150376607), AAV-PAEC8 (SEQ ID NO:25 of U.S. Pat. No. 20150376607), AAV-PAEC11 (SEQ ID NO:26 of U.S. Pat. No. 20150376607), AAV-PAEC12 (SEQ ID NO:27, of U.S. Pat. No. 20150376607), or variants thereof.

In certain embodiments, the AAV serotype may be, or comprise, a sequence as described in U.S. Pat. No. 9,163,261 (the content of which is incorporated herein by reference in its entirety as related to AAV capsids, insofar as it does not conflict with the present disclosure), such as, but not limited to, AAV-2-pre-miRNA-101 (SEQ ID NO: 1 U.S. Pat. No. 9,163,261), or variants thereof.

In certain embodiments, the AAV serotype may be, or have, a sequence as described in U.S. Patent Application Publication No. U.S. Pat. No. 20150376240 (the content of which is incorporated herein by reference in its entirety as related to AAV capsids, insofar as it does not conflict with the present disclosure), such as, but not limited to, AAV-8h (SEQ ID NO: 6 of U.S. Pat. No. 20150376240), AAV-8b (SEQ ID NO: 5 of U.S. Pat. No. 20150376240), AAV-h (SEQ ID NO: 2 of U.S. Pat. No. 20150376240), AAV-b (SEQ ID NO: 1 of U.S. Pat. No. 20150376240), or variants thereof

In certain embodiments, the AAV serotype may be, or have, a sequence as described in U.S. Patent Application Publication No. US20160017295 (the content of which is incorporated herein by reference in its entirety as related to AAV capsids, insofar as it does not conflict with the present disclosure), such as, but not limited to, AAV SM 10-2 (SEQ ID NO: 22 of US20160017295), AAV Shuffle 100-1 (SEQ ID NO: 23 of US20160017295), AAV Shuffle 100-3 (SEQ ID NO: 24 of US20160017295), AAV Shuffle 100-7 (SEQ ID NO: 25 of US20160017295), AAV Shuffle 10-2 (SEQ ID NO: 34 of US20160017295), AAV Shuffle 10-6 (SEQ ID NO: 35 of US20160017295), AAV Shuffle 10-8 (SEQ NO: 36 of US20160017295), AAV Shuffle 100-2 (SEQ ID NO: 37 of US20160017295), AAV SM 10-1 (SEQ ID NO: 38 of US20160017295), AAV SM 10-8 (SEQ ID NO: 39 of US20160017295), AAV SM 100-3 (SEQ ID NO: 40 of US20160017295), AAV SM 100-10 (SEQ ID NO: 41 of US20160017295), or variants thereof.

In certain embodiments, the AAV serotype may he, or comprise, a sequence as described in United States Patent Publication No. U.S. Pat. No. 20150238550 (the content of which is incorporated herein by reference in its entirety as related to AAV capsids, insofar as it does not conflict with the present disclosure), such as, but not limited to, BNP61 AAV (SEQ NO: 1 of U.S. Pat. No. 20150238550), BNP62 AAV (SEQ ID NO: 3 of U.S. Pat. No. 20150238550), BNP63 AAV (SEQ ID NO: 4 of U.S. Pat. No. 20150238550), or variants thereof.

In certain embodiments, the AAV serotype may be or may comprise a sequence as described in United States Patent Publication No. U.S. Pat. No. 20150315612 (the content of which is incorporated herein by reference in its entirety as related to AAV capsids, insofar as it does not conflict with the present disclosure), such as, but not limited to, AAVrh.50 (SEQ ID NO: 108 of U.S. Pat. No. 20150315612), AAVrh.43 (SEQ ID NO: 163 of U.S. Pat. No. 20150315612), AAVrh.62 (SEQ ID NO: 114 of U.S. Pat. No. 20150315612), AAVrh.48 (SEQ ID NO: 115 of U.S. Pat. No. 20150315612), AAVhu.19 (SEQ ID NO: 133 of U.S. Pat. No. 20150315612), AAVhu.11 (SEQ ID NO: 153 of U.S. Pat. No. 20150315612), AAVhu.53 (SEQ ID NO: 186 of U.S. Pat. No. 20150315612), AAV4-8/rh.64 (SEQ ID No: 15 of U.S. Pat. No. 20150315612)-AAVLG-9/hu.39 (SEQ ID No: 24 of U.S. Pat. No. 20150315612), AAV54.5/hu.23 (SEQ ID No: 60 of U.S. Pat. No. 20150315612), AAV54.2/1m.22 (SEQ ID No: 67 of U.S. Pat. No. 20150315612), AAV54.7/1ru.24 (SEQ ID No: 66 of U.S. Pat. No. 20150315612), AAV54.1/hu.21 (SEQ ID No: 65 of U.S. Pat. No. 20150315612), AAV54.4R/hu.27 (SEQ ID No: 64 of U.S. Pat. No. 20150315612), AAV46.2/hu.28 (SEQ ID No: 68 of U.S. Pat. No. 20150315612), AAV46.6/hu.29 (SEQ ID No: 69 of U.S. Pat. No. 20150315612), AAV128.1./hu.43 (SEQ ID No: 80 of U.S. Pat. No. 20150315612), or variants thereof.

In certain embodiments, the AAV serotype may be, or comprise, a sequence as described in International Publication No. WO2015121501 (the content of which is incorporated herein by reference in its entirety as related to AAV capsids, insofar as it does not conflict with the present disclosure), such as, but not limited to, true type AAV (ttAAV) (SEQ ID NO: 2 of WO2015121501), “UPenn AAV10” (SEQ ID NO: 8 of WO2015121501) “Japanese AAV10” (SEQ ID NO: 9 of WO2015121501), or variants thereof

According to the present disclosure, AAV capsid serotype selection or use may be from a variety of species. In certain embodiments, the AAV may be an avian AAV (AAAV). The AAAV serotype may be, or have, a sequence as described in U.S. Pat. No. 9,238,800 (the content of which is incorporated herein by reference in its entirety as related to AAV capsids, insofar as it does not conflict with the present disclosure), such as, but not limited to, AAAV (SEQ ID NO: 1, 2, 4, 6, 8, 10, 12, or 14 of U.S. Pat. No. 9,238,800), or variants thereof.

In certain embodiments, the AAV may be a bovine AAV (BAAV). The BAAV serotype may be, or have, a sequence as described in U.S. Pat. No. 9,193,769 (the content of which is incorporated herein by reference in its entirety as related to AAV capsids, insofar as it does not conflict with the present disclosure), such as, but not limited to. BAAV (SEQ ID NO: 1 and 6 of U.S. Pat. No. 9,193,769), or variants thereof. The BAAV serotype may be or have a sequence as described in U.S. Pat. No. 7,427,396 (the content of which is incorporated herein by reference in its entirety as related to AAV capsids, insofar as it does not conflict with the present disclosure), such as, but not limited to, BAAV (SEQ ID NO: 5 and 6 of U.S. Pat. No. 7,427,396), or variants thereof.

In certain embodiments, the AAV may be a caprine AAV. The caprine AAV serotype may be, or have, a sequence as described in U.S. Pat. No. 7,427,396 (the content of which is incorporated herein by reference in its entirety as related to AAV capsids, insofar as it does not conflict with the present disclosure), such as, but not limited to, caprine AAV (SEQ ID NO: 3 of U.S. Pat. No. 7,427,396), or variants thereof.

In certain embodiments, the AAV may be engineered as a hybrid AAV from two or more parental serotypes. In certain embodiments, the AAV may be AAV2G9 which comprises sequences from AAV2 and AA.V9. The AAV2G9 AAV serotype may be, or have, a sequence as described in U.S. Patent Application Publication No. US20160017005 (the content of which is incorporated herein by reference in its entirety as related to AAV capsids, insofar as it does not conflict with the present disclosure).

In certain embodiments, the AAV may be a serotype generated by the AAV9 capsid library with mutations in amino acids 390-627 (VP1 numbering) as described by Pulicheda et al. (Molecular Therapy 19(6):1070-1078 (2011) (the content of which is incorporated herein by reference in its entirety as related to AAV capsids, insofar as it does not conflict with the present disclosure). The serotype and corresponding nucleotide and amino acid substitutions may be, but is not limited to, AAV9.1 (G-1594C; D53214), AAV6.2 (T1418A and 71436X; V473D and 1479K), AAV9.3 (T1238A; F413Y), AAV9.4 (11250C and A16177; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A14251, A1702C, A17691; 1568P, Q590L), AAV9.13 (A1369C, A1720T; N457H, T574S), AAV9.14 (T1.340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A; G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, 11806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L), or AAV9.95 (T1605A; F535L).

In certain embodiments, the AAV serotype nay he, or comprise, a sequence as described in International Publication No. WO2016049230 (the content of which is incorporated herein by reference in its entirety as related to AAV capsids, insofar as it does not conflict with the present disclosure), such as, but not limited to AAVF1/HSC1 (SEQ ID NO: 2 and 20 of WO2016049230)-.NAVF2/HSC2 (SEQ ID NO: 3 and 21 of WO2016049230), AAVF3/HSC3 (SEQ m NO: 5 and 22 of WO2016049230), AAVF4/HSC4 (SEQ ID NO: 6 and 23 of WO2016049230), AAVF8/HSC5 (SEQ ID NO: 11 and 25 of WO2016049230), AAVF6/HSC6 (SEQ ID NO: 7 and 24 of WO2016049230), AAVF7/HSC7 (SEQ ID NO: 8 and 27 of WO2016049230), AAVF8/HSC8 (SEQ ID NO: 9 and 28 of WO2016049230), AAVF9/HSC9 (SEQ ID NO: 10 and 29 of WO2016049230), AAVF11/HSC11 (SEQ ID NO: 4 and 26 of WO2016049230), ANVF12/HSC12 (SEQ ID NO: 12 and 30 of WO2016049230), AAVF13/HSC13 (SEQ ID NO: 14 and 31 of WO2016049230), AAVF14/HSC14 (SEQ ID NO: 15 and 32 of WO2016049230), AAVF15/HSC15 (SEQ ID NO: 16 and 33 of WO2016049230), AAVF16/HSC16 (SEQ ID NO: 17 and 34 of WO2016049230), AAVF17/HSC17 (SEQ ID NO: 13 and 35 of WO2016049230), or variants or derivatives thereof.

In certain embodiments, the AAV serotype may he, or comprise, a sequence as described in U.S. Pat. No. 8,734,809 (the content of which is incorporated herein by reference in its entirety as related to AAV capsids, insofar as it does not conflict with the present disclosure), such as, but not limited to, AAV CBr-E1 (SEQ ID NO: 13 and 87 of U.S. Pat. No. 8,734,809), AAV CBr-E2 (SEQ ID NO: 14 and 88 of U.S. Pat. No. 8,734,809), AAV CBr-E3 (SEQ ID NO: 15 and 89 of U.S. Pat. No. 8,734,809), AAV CBr-E4 (SEQ ID NO: 16 and 90 of U.S. Pat. No. 8,734,809), AAV CBr-E5 (SEQ ID NO: 17 and 91 of U.S. Pat. No. 8,734,809), AAV CBr-e5 (SEQ ID NO: 18 and 92 of U.S. Pat. No. 8,734,809), AAV CBr-E6 (SEQ ID NO: 19 and 93 of U.S. Pat. No. 8,734,809), AAV CBr-E7 (SEQ ID NO: 20 and 94 of U.S. Pat. No. 8,734,809), AAV CBr-E8 (SEQ ID NO: 21 and 95 of U.S. Pat. No. 8,734,809), AAV CLv-D1 (SEQ ID NO: 22 and 96 of U.S. Pat. No. 8,734,809), AAV CLv-D2 (SEQ ID NO: 23 and 97 of U.S. Pat. No. 8,734,809), AAV CLv-D3 (SEQ ID NO: 24 and 98 of U.S. Pat. No. 8,734,809), AAV (SEQ ID NO: 25 and 99 of U.S. Pat. No. 8,734,809), AAV CLv-D5 (SEQ ID NO: 26 and 100 of U.S. Pat. No. 8,734,809), AAV CLv-D6 (SEQ ID NO: 27 and 101 of U.S. Pat. No. 8,734,809), AAV CLv-D7 (SEQ ID NO: 28 and 102 of U.S. Pat. No. 8,734,809), AAV CLv-D8 (SEQ ID NO: 29 and 103 of U.S. Pat. No. 8,734,809), AAV CLv-E1 (SEQ ID NO: 13 and 87 of U.S. Pat. No. 8,734,809), AAV CLv-R1 (SEQ ID NO: 30 and 104 of U.S. Pat. No. 8,734,809), AAV CLv-R2 (SEQ ID NO: 31 and 105 of U.S. Pat. No. 8,734,809), AAV CLv-R3 (SEQ ID NO: 32 and 106 of U.S. Pat. No. 8,734,809), AAV CLv-R4 (SEQ ID NO: 33 and 107 of U.S. Pat. No. 8,734,809), AAV CLv-R5 (SEQ ID NO: 34 and 108 of U.S. Pat. No. 8,734,809), AAV CLv-R6 (SEQ ID NO: 35 and 109 of U.S. Pat. No. 8,734,809), AAV CLv-R7 (SEQ ID NO: 36 and 110 of U.S. Pat. No. 8,734,809), AAV CLv-R8 (SEQ ID NO: 37 and 111 of U.S. Pat. No. 8,734,809), AAV CLv-R9 (SEQ ID NO: 38 and 112 of U.S. Pat. No. 8,734,809), AAV CLg-F1 (SEQ ID NO: 39 and 113 of U.S. Pat. No. 8,734,809), AAV CLg-F2 (SEQ ID NO: 40 and 114 of U.S. Pat. No. 8,734,809), AAV CLg-F3 (SEQ ID NO: 41 and 115 of U.S. Pat. No. 8,734,809), AAV CLg-F4 (SEQ ID NO: 42 and 116 of U.S. Pat. No. 8,734,809), AAV CLg-F5 (SEQ ID NO: 43 and 117 of U.S. Pat. No. 8,734,809), AAV CLg-F6 (SEQ ID NO: 43 and 117 of U.S. Pat. No. 8,734,809), AAV CLg-F7 (SEQ ID NO: 44 and 118 of U.S. Pat. No. 8,734,809), AAV CLg-F8 (SEQ ID NO: 43 and 117 of U.S. Pat. No. 8,734,809), AAV CSp-1 (SEQ ID NO: 45 and 119 of U.S. Pat. No. 8,734,809), AAV CSp-10 (SEQ ID NO: 46 and 120 of U.S. Pat. No. 8,734,809), AAV CSp-11 (SEQ ID NO: 47 and 121 of U.S. Pat. No. 8,734,809), AAV CSp-2 (SEQ ID NO: 48 and 122 of U.S. Pat. No. 8,734,809), AAV CSp-3 (SEQ ID NO: 49 and 123 of U.S. Pat. No.8,734,809), AAV CSp-4 (SEQ ID NO: 50 and 124 of U.S. Pat. No. 8,734,809), AAV CSp-6 (SEQ ID NO: 51 and 125 of U.S. Pat. No. 8,734,809), AAV CSp-7 (SEQ ID NO: 52 and 126 of U.S. Pat. No. 8,734,809), AAV CSp-8 (SEQ ID NO: 53 and 127 of U.S. Pat. No. 8,734,809), AAV CSp-9 (SEQ NO: 54 and 128 of U.S. Pat. No. 8,734,809), AAV CHt-2 (SEQ ID NO: 55 and 129 of U.S. Pat. No. 8,734,809), AAV CHt-3 (SEQ ID NO: 56 and 130 of U.S. Pat. No. 8,734,809), AAV CKd-1 (SEQ ID NO: 57 and 131 of U.S. Pat. No. 8,734,809), AAV CKd-10 (SEQ ID NO: 58 and 132 of U.S. Pat. No. 8,734,809), AAV CKd-2 (SEQ ID NO: 59 and 133 of U.S. Pat. No. 8,734,809), AAV CKd-3 (SEQ ID NO: 60 and 134 of U.S. Pat. No. 8,734,809), AAV CKd-4 (SEQ ID NO: 61 and 135 of U.S. Pat. No. 8,734,809), AAV CKd-6 (SEQ ID NO: 62 and 136 of U.S. Pat. No. 8,734,809), AAV CKd-7 (SEQ ID NO: 63 and 137 of U.S. Pat. No. 8,734,809), AAV CKd-8 (SEQ ID NO: 64 and 138 of U.S. Pat. No. 8,734,809), AAV CLv-1 (SEQ ID NO: 35 and 139 of U.S. Pat. No. 8,734,809), AAV CLv-12 (SEQ ID NO: 66 and 140 of U.S. Pat. No. 8,734,809), AAV CLv-13 (SEQ ID NO: 67 and 141 of U.S. Pat. No. 8,734,809), AAV CLv-2 (SEQ ID NO: 68 and 142 of U.S. Pat. No. 8,734,809), AAV CLv-3 (SEQ ID NO: 69 and 143 of U.S. Pat. No. 8,734,809), AAV CLv-4 (SEQ NO: 70 and 144 of U.S. Pat. No. 8,734,809), AAV CLv-6 (SEQ ID NO: 71 and 145 of U.S. Pat. No. 8,734,809), AAV CLv-8 (SEQ ID NO: 72 and 146 of U.S. Pat. No. 8,734,809), AAV CKd-B1 (SEQ ID NO: 73 and 147 of U.S. Pat. No. 8,734,809), AAV CKd-B2 (SEQ ID NO: 74 and 148 of U.S. Pat. No. 8,734,809), AAV CKd-B3 (SEQ ID NO: 75 and 149 of U.S. Pat. No. 8,734,809), AAV CKd-B4 (SEQ ID NO: 76 and 150 of U.S. Pat. No. 8,734,809), AAV CKd-B5 (SEQ ID NO: 77 and 151 of U.S. Pat. No. 8,734,809), AAV CKd-B6 (SEQ ID NO: 78 and 152 of U.S. Pat. No. 8,734,809), AAV CKd-B7 (SEQ ID NO: 79 and 153 of U.S. Pat. No. 8,734,809), AAV CKd-B8 (SEQ ID NO: 80 and 154 of U.S. Pat. No. 8,734,809), AAV CKd-H1 (SEQ ID NO: 81 and 155 of U.S. Pat. No. 8,734,809). AAV CKd-H2 (SEQ ID NO: 82 and 156 of U.S. Pat. No. 8,734,809), AAV CKd-H3 (SEQ ID NO: 83 and 157 of U.S. Pat. No. 8,734,809), AAV CKd-H4 (SEQ ID NO: 84 and 158 of U.S. Pat. No. 8,734,809), AAV CKd-HS (SEQ ID NO: 85 and 159 of U.S. Pat. No. 8,734,809), AAV CKd-H6 (SEQ ID NO: 77 and 151 of U.S. Pat. No. 8,734,809), AAV CHt-1 (SEQ ID NO: 86 and 160 of U.S. Pat. No. 8,734,809), AAV CLv1-1 (SEQ ID NO: 171 of U.S. Pat. No. 8,734,809), AAV CLv1-2 (SEQ ID NO: 172 of U.S. Pat. No. 8,734,809), AAV CLv1-3 (SEQ ID NO: 173 of U.S. Pat. No. 8,734,809), AAV CLv1-4 (SEQ ID NO: 174 of U.S. Pat. No. 8,734,809), AAV Clv1-7 (SEQ ID NO: 175 of U.S. Pat. No. 8,734,809), AAV Clv1-8 (SEQ ID NO: 176 of U.S. Pat. No. 8,734,809), AAV Clv1-9 (SEQ ID NO: 177 of U.S. Pat. No. 8,734,809), AAV Clv1-10 (SEQ ID NO: 178 of U.S. Pat. No. 8,734,809), AAV.VR-355 (SEQ ID NO: 181 of U.S. Pat. No. 8,734,809), AAV.hu.48R3 (SEQ ID NO: 183 of U.S. Pat. No. 8,734,809), or variants or derivatives thereof.

In certain embodiments, the AAV serotype may be, or comprise, a sequence as described in International Publication No. WO2016065001 (the content of which is incorporated herein by reference in its entirety as related to AAV capsids, insofar as it does not conflict with the present disclosure), such as, but not limited to AAV CHt-P2 (SEQ ID NO: 1 and 51 of WO2016065001), AAV CHt-P5 (SEQ ID NO: 2 and 52 of WO2016065001), AAV CHt-P9 (SEQ ID NO: 3 and 53 of WO2016065001), AAV CBr-7.1 (SEQ ID NO: 4 and 54 of WO2016065001), AAV CBr-7.2 (SEQ ID NO: 5 and 55 of WO2016065001), AAV CBr-7.3 (SEQ ID NO: 6 and 56 of WO2016065001), AAV CBr-7.4 (SEQ ID NO: 7 and 57 of WO2016065001), AAV CBr-7.5 (SEQ ID NO: 8 and 58 of WO2016065001), AAV CBr-7.7 (SEQ ID NO: 9 and 59 of WO2016065001), AAV CBr-7.8 (SEQ ID NO: 10 and 60 of WO2016065001), AAV CBr-7.10 (SEQ ID NO: 11 and 61 of WO2016065001), AAV CKd-N3 (SEQ m NO: 12 and 62 of WO2016065001), AAV CKd-N4 (SEQ ID NO: 13 and 63 of WO2016065001), AAV CKd-N9 (SEQ 1113 NO: 14 and 64 of WO2016065001), AAV CLv-L4 (SEQ ID NO: 15 and 65 of WO2016065001), AAV CLv-L5 (SEQ ID NO: 16 and 66 of WO2016065001), AAV CLv-L6 (SEQ ID NO: 17 and 67 of WO2016065001), AAV CLv-K1 (SEQ ID NO: 18 and 68 of WO2016065001), AAV CLv-K3 (SEQ ID NO: 19 and 69 of WO2016065001), AAV (SEQ ID NO: 20 and 70 of WO2016065001), AAV CLv-M1 (SEQ ID NO: 21 and 71 of WO2016065001), AAV CLv-M11 (SEQ ID NO: 22 and 72 of WO2016065001), AAV CLv-M2 (SEQ ID NO: 23 and 73 of WO2016065001), AAV CLv-M5 (SEQ ID NO: 24 and 74 of WO2016065001), AAV CLv-M6 (SEQ ID NO: 25 and 75 of WO2016065001), AAV CLv-M7 (SEQ ID NO: 26 and 76 of WO2016065001), AAV CLv-M8 (SEQ ID NO: 27 and 77 of WO2016065001), AAV CLv-M9 (SEQ ID NO: 28 and 78 of WO2016065001), AAV CHt-P1 (SEQ ID NO: 29 and 79 of WO2016065001), AAV CLv-P6 (SEQ ID NO: 30 and 80 of WO2016065001), AAV CHt-P8 (SEQ ID NO: 31 and 81 of WO2016065001), AAV CHt-6.1 (SEQ ID NO: 32 and 82 of WO2016065001), AAV CHt-6.10 (SEQ ID NO: 33 and 83 of WO2016065001), AAV CHt-6.5 (SEQ ID NO: 34 and 84 of WO2016065001), AAV CHt-6.6 (SEQ ID NO: 35 and 85 of WO2016065001), AAV CHt-6.7 (SEQ ID NO: 36 and 86 of WO2016065001), AAV CHt-6.8 (SEQ ID NO: 37 and 87 of WO2016065001), AAV CSp-8.10 (SEQ ID NO: 38 and 88 of WO2016065001), AAV CSp-8.2 (SEQ ID NO: 39 and 89 of WO2016065001), AAV CSp-8.4 (SEQ ID NO: 40 and 90 of WO2016065001), AAV CSp-8.5 (SEQ ID NO: 41 and 91 of WO2016065001), AAV CSp-8.6 (SEQ ID NO: 42 and 92 of WO2016065001)-NAV CSp-8.7 (SEQ ID NO: 43 and 93 of WO2016065001), AAV CSp-8.8 (SEQ ID NO: 44 and 94 of WO2016065001), AAV CSp-8.9 (SEQ ID NO: 45 and 95 of WO2016065001), AAV CBr-B7.3 (SEQ ID NO: 46 and 96 of WO2016065001), AAV CBr-B7.4 (SEQ ID NO: 47 and 97 of WO2016065001), AAV3B (SEQ ID NO: 48 and 98 of WO2016065001), AAV4 (SEQ NO: 49 and 99 of WO 016065001), AAV5 (SEQ ID NO: 50 and 100 of WO2016065001), or variants or derivatives thereof.

In certain embodiments, the AAV particle may be or comprise a serotype selected from any of those found in Table 1.

In certain embodiments, the AAV particle may comprise a sequence, fragment, or variant of any sequence in Table 1.

In certain embodiments, the AAV particle may be encoded by a sequence, fragment, or variant of any sequence in Table 1.

In the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (On) for Glutamine; E (Glu) for Glutamic Acid; S (Ser) for Serine; P (Pro) for Prolific; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (A.sx) for Aspartic acid or: sparathne; (Xle) for Leucine or Isoleucine; O (Pyl) for Pyrrolysine; U (Sec) for Selenocysteine; X (Xaa) for any amino acid; and Z (Glx) for Glutamine or Glutamic acid.

TABLE 1
Representative AAV Serotypes

| Serotype | SEQ ID NO | Reference Information |
|---|---|---|
| VOY101 | 1 or 1722 | — |
| VOY201 | 1723 or 1724 | — |
| PHP.N/PHP.B-DGT | 7 | WO2017100671 SEQ ID NO: 46 |
| AAVPHP.B or G2B-26 | 3 | WO2015038958 SEQ ID NO: 8 and 13 |
| AAVPHP.B | 4 | WO2015038958 SEQ ID NO: 9 |
| AAVG2B-13 | 5 | WO2015038958 SEQ ID NO: 12 |
| AAVTH1.1-32 | 6 | WO2015038958 SEQ ID NO: 14 |
| AAVTH1.1-35 | 7 | WO2015038958 SEQ ID NO: 15 |
| PHP.S/G2A12 | 8 | WO2017100671 SEQ ID NO: 47 |
| AAV9/hu.14 K449R | 9 | WO2017100671 SEQ ID NO: 45 |
| AAV1 | 10 | US20150159173 SEQ ID NO: 11, |
| | | US20150315612 SEQ ID NO: 202 |
| AAV1 | 11 | US20160017295 SEQ ID NO: 1, US20030138772 |
| | | SEQ ID NO: 64, US20150159173 SEQ ID NO: 27, |
| | | US20150315612 SEQ ID NO: 219, US7198951 |
| | | SEQ ID NO: 5 |
| AAV1 | 12 | US20030138772 SEQ ID NO: 6 |
| AAV1.3 | 13 | US20030138772 SEQ ID NO: 14 |
| AAV10 | 14 | US20030138772 SEQ ID NO: 117 |
| AAV10 | 15 | WO2015121501 SEQ ID NO: 9 |
| AAV10 | 16 | WO2015121501 SEQ ID NO: 8 |
| AAV11 | 17 | US20030138772 SEQ ID NO: 118 |
| AAV12 | 18 | US20030138772 SEQ ID NO: 119 |
| AAV2 | 19 | US20150159173 SEQ ID NO: 7, |
| | | US20150315612 SEQ ID NO: 211 |
| AAV2 | 20 | US20030138772 SEQ ID NO: 70, US20150159173 |
| | | SEQ ID NO: 23, US20150315612 SEQ ID NO: 221, |
| | | US20160017295 SEQ ID NO: 2, US6156303 SEQ ID |
| | | NO: 4, US7198951 SEQ ID NO: 4, WO2015121501 |
| | | SEQ ID NO: 1 |
| AAV2 | 21 | US6156303 SEQ ID NO: 8 |
| AAV2 | 22 | US20030138772 SEQ ID NO: 7 |
| AAV2 | 23 | US6156303 SEQ ID NO: 3 |
| AAV2.5T | 24 | US923313I SEQ ID NO: 42 |
| AAV223.10 | 25 | US20030138772 SEQ ID NO: 75 |
| AAV223.2 | 26 | US20030138772 SEQ ID NO: 49 |
| AAV223.2 | 27 | US20030138772 SEQ ID NO: 76 |
| AAV223.4 | 28 | US20030138772 SEQ ID NO: 50 |
| AAV223.4 | 29 | US20030138772 SEQ ID NO: 73 |
| AAV223.5 | 30 | US20030138772 SEQ ID NO: 51 |
| AAV223.5 | 31 | US20030138772 SEQ ID NO: 74 |
| AAV223.6 | 32 | US20030138772 SEQ ID NO: 52 |
| AAV223.6 | 33 | US20030138772 SEQ ID NO: 78 |
| AAV223.7 | 34 | US20030138772 SEQ ID NO: 53 |
| AAV223.7 | 35 | US20030138772 SEQ ID NO: 77 |
| AAV29.3 | 36 | US20030138772 SEQ ID NO: 82 |
| AAV29.4 | 37 | US20030138772 SEQ ID NO: 12 |
| AAV29.5 | 38 | US20030138772 SEQ ID NO: 83 |
| AAV29.5 (AAVbb.2) | 39 | US20030138772 SEQ ID NO: 13 |
| AAV3 | 40 | US20150159173 SEQ ID NO: 12 |
| AAV3 | 41 | US20030138772 SEQ ID NO: 71, US20150159173 |
| | | SEQ ID NO: 28, US20160017295 SEQ ID NO: 3, |
| | | US7198951 SEQ ID NO: 6 |
| AAV3 | 42 | US20030138772 SEQ ID NO: 8 |
| AAV3.3b | 43 | US20030138772 SEQ ID NO: 72 |
| AAV3-3 | 44 | US20150315612 SEQ ID NO: 200 |
| AAV3-3 | 45 | US20150315612 SEQ ID NO: 217 |
| AAV3a | 46 | US6156303 SEQ ID NO: 5 |
| AAV3a | 47 | US6156303 SEQ ID NO: 9 |
| AAV3b | 48 | US6156303 SEQ ID NO: 6 |
| AAV3b | 49 | US6156303 SEQ ID NO: 10 |
| AAV3b | 50 | US6156303 SEQ ID NO: 1 |
| AAV4 | 51 | US20140348794 SEQ ID NO: 17 |
| AAV4 | 52 | US20140348794 SEQ ID NO: 5 |
| AAV4 | 53 | US20140348794 SEQ ID NO: 3 |
| AAV4 | 54 | US20140348794 SEQ ID NO: 14 |
| AAV4 | 55 | US20140348794 SEQ ID NO: 15 |
| AAV4 | 56 | US20140348794 SEQ ID NO: 19 |
| AAV4 | 57 | US20140348794 SEQ ID NO: 12 |
| AAV4 | 58 | US20140348794 SEQ ID NO: 13 |
| AAV4 | 59 | US20140348794 SEQ ID NO: 7 |
| AAV4 | 60 | US20140348794 SEQ ID NO: 8 |
| AAV4 | 61 | US20140348794 SEQ ID NO: 9 |
| AAV4 | 62 | US20140348794 SEQ ID NO: 2 |
| AAV4 | 63 | US20140348794 SEQ ID NO: 10 |
| AAV4 | 64 | US20140348794 SEQ ID NO: 11 |
| AAV4 | 65 | US20140348794 SEQ ID NO: 18 |
| AAV4 | 66 | US20030138772 SEQ ID NO: 63, US20160017295 |
| | | SEQ ID NO: 4, US20140348794 SEQ ID NO: 4 |
| AAV4 | 67 | US20140348794 SEQ ID NO: 16 |
| AAV4 | 68 | US20140348794 SEQ ID NO: 20 |
| AAV4 | 69 | US20140348794 SEQ ID NO: 6 |
| AAV4 | 70 | US20140348794 SEQ ID NO: 1 |
| AAV42.2 | 71 | US20030138772 SEQ ID NO: 9 |
| AAV42.2 | 72 | US20030138772 SEQ ID NO: 102 |
| AAV42.3b | 73 | US20030138772 SEQ ID NO: 36 |
| AAV42.3B | 74 | US20030138772 SEQ ID NO: 107 |
| AAV42.4 | 75 | US20030138772 SEQ ID NO: 33 |
| AAV42.4 | 76 | US20030138772 SEQ ID NO: 88 |
| AAV42.8 | 77 | US20030138772 SEQ ID NO: 27 |
| AAV42.8 | 78 | US20030138772 SEQ ID NO: 85 |
| AAV43.1 | 79 | US20030138772 SEQ ID NO: 39 |
| AAV43.1 | 80 | US20030138772 SEQ ID NO: 92 |
| AAV43.12 | 81 | US20030138772 SEQ ID NO: 41 |
| AAV43.12 | 82 | US20030138772 SEQ ID NO: 93 |
| AAV43.20 | 83 | US20030138772 SEQ ID NO: 42 |
| AAV43.20 | 84 | US20030138772 SEQ ID NO: 99 |
| AAV43.21 | 85 | US20030138772 SEQ ID NO: 43 |
| AAV43.21 | 86 | US20030138772 SEQ ID NO: 96 |
| AAV43.23 | 87 | US20030138772 SEQ ID NO: 44 |
| AAV43.23 | 88 | US20030138772 SEQ ID NO: 98 |
| AAV43.25 | 89 | US20030138772 SEQ ID NO: 45 |
| AAV43.25 | 90 | US20030138772 SEQ ID NO: 97 |
| AAV43.5 | 91 | US20030138772 SEQ ID NO: 40 |
| AAV43.5 | 92 | US20030138772 SEQ ID NO: 94 |
| AAV4-4 | 93 | US20150315612 SEQ ID NO: 201 |
| AAV4-4 | 94 | US20150315612 SEQ ID NO: 218 |
| AAV44.1 | 95 | US20030138772 SEQ ID NO: 46 |
| AAV44.1 | 96 | US20030138772 SEQ ID NO: 79 |
| AAV44.5 | 97 | US20030138772 SEQ ID NO: 47 |
| AAV44.5 | 98 | US20030138772 SEQ ID NO: 80 |
| AAV4407 | 99 | US20150315612 SEQ ID NO: 90 |
| AAV5 | 100 | US7427396 SEQ ID NO: 1 |
| AAV5 | 101 | US20030138772 SEQ ID NO: 114 |
| AAV5 | 102 | US20160017295 SEQ ID NO: 5, US7427396 SEQ |
| | | ID NO: 2, US20150315612 SEQ ID NO: 216 |
| AAV5 | 103 | US20150315612 SEQ ID NO: 199 |
| AAV6 | 104 | US20150159173 SEQ ID NO: 13 |
| AAV6 | 105 | US20030138772 SEQ ID NO: 65, US20150159173 |
| | | SEQ ID NO: 29, US20160017295 SEQ ID NO: 6, |
| | | US6156303 SEQ ID NO: 7 |
| AAV6 | 106 | US6156303 SEQ ID NO: 11 |
| AAV6 | 107 | US6156303 SEQ ID NO: 2 |
| AAV6 | 108 | US20150315612 SEQ ID NO: 203 |
| AAV6 | 109 | US20150315612 SEQ ID NO: 220 |
| AAV6.1 | 110 | US20150159173 |
| AAV6.12 | 111 | US20150159173 |
| AAV6.2 | 112 | US20150159173 |
| AAV7 | 113 | US20150159173 SEQ ID NO: 14 |
| AAV7 | 114 | US20150315612 SEQ ID NO: 183 |
| AAV7 | 115 | US20030138772 SEQ ID NO: 2, US20150159173 |
| | | SEQ ID NO: 30, US20150315612 SEQ ID NO: 181, |
| | | US20160017295 SEQ ID NO: 7 |
| AAV7 | 116 | US20030138772 SEQ ID NO: 3 |
| AAV7 | 117 | US20030138772 SEQ ID NO: 1, US20150315612 |
| | | SEQ ID NO: 180 |
| AAV7 | 118 | US20150315612 SEQ ID NO: 213 |
| AAV7 | 119 | US20150315612 SEQ ID NO: 222 |
| AAV8 | 120 | US20150159173 SEQ ID NO: 15 |
| AAV8 | 121 | US20150376240 SEQ ID NO: 7 |
| AAV8 | 122 | US20030138772 SEQ ID NO: 4, US20150315612 |
| | | SEQ ID NO: 182 |
| AAV8 | 123 | US20030138772 SEQ ID NO: 95, US20140359799 SEQ |
| | | ID NO: 1, US20150159173 SEQ ID NO: 31, |
| | | US20160017295 SEQ ID NO: 8, US7198951 SEQ ID |
| | | NO: 7, US20150315612 SEQ ID NO: 223 |
| AAV8 | 124 | US20150376240 SEQ ID NO: 8 |
| AAV8 | 125 | US20150315612 SEQ ID NO: 214 |
| AAV-8b | 126 | US20150376240 SEQ ID NO: 5 |
| AAV-8b | 127 | US20150376240 SEQ ID NO: 3 |
| AAV-8h | 128 | US20150376240 SEQ ID NO: 6 |
| AAV-8h | 129 | US20150376240 SEQ ID NO: 4 |
| AAV9 | 130 | US20030138772 SEQ ID NO: 5 |
| AAV9 | 131 | US7198951 SEQ ID NO: 1 |
| AAV9 | 132 | US20160017295 SEQ ID NO: 9 |
| AAV9 | 133 | US20030138772 SEQ ID NO: 100, US7198951 |
| | | SEQ ID NO: 2 |
| AAV9 | 134 | US7198951 SEQ ID NO: 3 |
| AAV9 (AAVhu.14) | 135 | US7906111 SEQ ID NO: 3; WO2015038958 |
| | | SEQ ID NO: 11 |
| AAV9 (AAVhu.14) | 136 | US7906111 SEQ ID NO: 123; WO2015038958 |
| | | SEQ ID NO: 2 |
| AAVA3.1 | 137 | US20030138772 SEQ ID NO: 120 |
| AAVA3.3 | 138 | US20030138772 SEQ ID NO: 57 |
| AAVA3.3 | 139 | US20030138772 SEQ ID NO: 66 |
| AAVA3.4 | 140 | US20030138772 SEQ ID NO: 54 |
| AAVA3.4 | 141 | US20030138772 SEQ ID NO: 68 |
| AAVA3.5 | 142 | US20030138772 SEQ ID NO: 55 |
| AAVA3.5 | 143 | US20030138772 SEQ ID NO: 69 |
| AAVA3.7 | 144 | US20030138772 SEQ ID NO: 56 |
| AAVA3.7 | 145 | US20030138772 SEQ ID NO: 67 |
| AAV29.3 (AAVbb.1) | 146 | US20030138772 SEQ ID NO: 11 |
| AAVC2 | 147 | US20030138772 SEQ ID NO: 61 |
| AAVCh.5 | 148 | US20150159173 SEQ ID NO: 46, US20150315612 |
| | | SEQ ID NO: 234 |
| AAVcy.2 (AAV13.3) | 149 | US20030138772 SEQ ID NO: 15 |
| AAV24.1 | 150 | US20030138772 SEQ ID NO: 101 |
| AAVcy.3 (AAV24.1) | 151 | US20030138772 SEQ ID NO: 16 |
| AAV27.3 | 152 | US20030138772 SEQ ID NO: 104 |
| AAVcy.4 (AAV27.3) | 153 | US20030138772 SEQ ID NO: 17 |
| AAVcy.5 | 154 | US20150315612 SEQ ID NO: 227 |
| AAV7.2 | 155 | US20030138772 SEQ ID NO: 103 |
| AAVcy.5 (AAV7.2) | 156 | US20030138772 SEQ ID NO: 18 |
| AAV16.3 | 157 | US20030138772 SEQ ID NO: 105 |
| AAVcy.6 (AAV16.3) | 158 | US20030138772 SEQ ID NO: 10 |
| AAVcy.5 | 159 | US20150159173 SEQ ID NO: 8 |
| AAVcy.5 | 160 | US20150159173 SEQ ID NO: 24 |
| AAVCy.5R1 | 161 | US20150159173 |
| AAVCy.5R2 | 162 | US20150159173 |
| AAVCy.5R3 | 163 | US20150159173 |
| AAVCy.5R4 | 164 | US20150159173 |
| AAVDJ | 165 | US20140359799 SEQ ID NO: 3, US7588772 |
| | | SEQ ID NO: 2 |
| AAVDJ | 166 | US20140359799 SEQ ID NO: 2, US7588772 |
| | | SEQ ID NO: 1 |
| AAVDJ-8 | 167 | US7588772; Grimm et al 2008 |
| AAVDJ-8 | 168 | US7588772, Grimm et at 2008 |
| AAVF5 | 169 | US20030138772 SEQ ID NO: 110 |
| AAVH2 | 170 | US20030138772 SEQ ID NO: 26 |
| AAVH6 | 171 | US20030138772 SEQ ID NO: 25 |
| AAVhE1.1 | 172 | US9233131 SEQ ID NO: 44 |
| AAVhEr1.14 | 173 | US9233131 SEQ ID NO: 46 |
| AAVhEr1.16 | 174 | US9233131 SEQ ID NO: 48 |
| AAVhEr1.18 | 175 | US9233131 SEQ ID NO: 49 |
| AAVhEr1.23 (AAVhEr2.29) | 176 | US9233131 SEQ ID NO: 53 |
| AAVhEr1.35 | 177 | US9233131 SEQ ID NO: 50 |
| AAVhEr1.36 | 178 | US9233131 SEQ ID NO: 52 |
| AAVhEr1.5 | 179 | US9233131 SEQ ID NO: 45 |
| AAVhEr1.7 | 180 | US9233131 SEQ ID NO: 51 |
| AAVhEr1.8 | 181 | US9233131 SEQ ID NO: 47 |
| AAVhEr2.16 | 182 | US9233131 SEQ ID NO: 55 |
| AAVhEr2.30 | 183 | US9233131 SEQ ID NO: 56 |
| AAVhEr2.31 | 184 | US9233131 SEQ ID NO: 58 |
| AAVhEr2.36 | 185 | US9233131 SEQ ID NO: 57 |
| AAVhEr2.4 | 186 | US9233131 SEQ ID NO: 54 |
| AAVhEr3.1 | 187 | US9233131 SEQ ID NO: 59 |
| AAVhu.1 | 188 | US20150315612 SEQ ID NO: 46 |
| AAVhu.1 | 189 | US20150315612 SEQ ID NO: 144 |
| AAVhu.10 (AAV16.8) | 190 | US20150315612 SEQ ID NO: 56 |
| AAVhu.10 (AAV16.8) | 191 | US20150315612 SEQ ID NO: 156 |
| AAVhu.11 (AAV16.12) | 192 | US20150315612 SEQ ID NO: 57 |
| AAVhu.11 (AAV16.12) | 193 | US20150315612 SEQ ID NO: 153 |
| AAVhu.12 | 194 | US20150315612 SEQ ID NO: 59 |
| AAVhu.12 | 195 | US20150315612 SEQ ID NO: 154 |
| AAVhu.13 | 196 | US20150159173 SEQ ID NO: 16, US20150315612 |
| | | SEQ ID NO: 71 |
| AAVhu.13 | 197 | US20150159173 SEQ ID NO: 32, US20150315612 |
| | | SEQ ID NO: 129 |
| AAVhu.136.1 | 198 | US20150315612 SEQ ID NO: 165 |
| AAVhu.140.1 | 199 | US20150315612 SEQ ID NO: 166 |
| AAVhu.140.2 | 200 | US20150315612 SEQ ID NO: 167 |
| AAVhu.145.6 | 201 | US20150315612 SEQ ID No: 178 |
| AAVhu.15 | 202 | US20150315612 SEQ ID NO: 147 |
| AAVhu.15 (AAV33.4) | 203 | US20150315612 SEQ ID NO: 50 |
| AAVhu.156.1 | 204 | US20150315612 SEQ ID No: 179 |
| AAVhu.16 | 205 | US20150315612 SEQ ID NO: 148 |
| AAVhu.16 (AAV33.8) | 206 | US20150315612 SEQ ID NO: 51 |
| AAVhu.17 | 207 | US20150315612 SEQ ID NO: 83 |
| AAVhu.17 (AA V33.12) | 208 | US20150315612 SEQ ID NO: 4 |
| AAVhu.172.1 | 209 | US20150315612 SEQ ID NO: 171 |
| AAVhu.172.2 | 210 | US20150315612 SEQ ID NO: 172 |
| AAVhu.173.4 | 211 | US20150315612 SEQ ID NO: 173 |
| AAVhu.173.8 | 212 | US20150315612 SEQ ID NO: 175 |
| AAVhu.18 | 213 | US20150315612 SEQ ID NO: 52 |
| AAVhu.18 | 214 | US20150315612 SEQ ID NO: 149 |
| AAVhu.19 | 215 | US20150315612 SEQ ID NO: 62 |
| AAVhu.19 | 216 | US20150315612 SEQ ID NO: 133 |
| AAVhu.2 | 217 | US20150315612 SEQ ID NO: 48 |
| AAVhu.2 | 218 | US20150315612 SEQ ID NO: 143 |
| AAVhu.20 | 219 | US20150315612 SEQ ID NO: 63 |
| AAVhu.20 | 220 | US20150315612 SEQ ID NO: 134 |
| AAVhu.21 | 221 | US20150315612 SEQ ID NO: 65 |
| AAVhu.21 | 222 | US20150315612 SEQ ID NO: 135 |
| AAVhu.22 | 223 | US20150315612 SEQ ID NO: 67 |
| AAVhu.22 | 224 | US20150315612 SEQ ID NO: 138 |
| AAVhu.23 | 225 | US20150315612 SEQ ID NO: 60 |
| AAVhu.23.2 | 226 | US20150315612 SEQ ID NO: 137 |
| AAVhu.24 | 227 | US20150315612 SEQ ID NO: 66 |
| AAVhu.24 | 228 | US20150315612 SEQ ID NO: 136 |
| AAVhu.25 | 229 | US20150315612 SEQ ID NO: 49 |
| AAVhu.25 | 230 | US20150315612 SEQ ID NO: 146 |
| AAVhu.26 | 231 | US20150159173 SEQ ID NO: 17, US20150315612 |
| | | SEQ ID NO: 61 |
| AAVhu.26 | 232 | US20150159173 SEQ ID NO: 33, US20150315612 |
| | | SEQ ID NO: 139 |
| AAVhu.27 | 233 | US20150315612 SEQ ID NO: 64 |
| AAVhu.27 | 234 | US20150315612 SEQ ID NO: 140 |
| AAVhu.28 | 235 | US20150315612 SEQ ID NO: 68 |
| AAVhu.28 | 236 | US20150315612 SEQ ID NO: 130 |
| AAVhu.29 | 237 | US20150315612 SEQ ID NO: 69 |
| AAVhu.29 | 238 | US20150159173 SEQ ID NO: 42, US20150315612 |
| | | SEQ ID NO: 132 |
| AAVhu.29 | 239 | US20150315612 SEQ ID NO: 225 |
| AAVhu.29R | 240 | US20150159173 |
| AAVhu.3 | 241 | US20150315612 SEQ ID NO: 44 |
| AAVhu.3 | 242 | US20150315612 SEQ ID NO: 145 |
| AAVhu.30 | 243 | US20150315612 SEQ ID NO: 70 |
| AAVhu.30 | 244 | US20150315612 SEQ ID NO: 131 |
| AAVhu.31 | 245 | US20150315612 SEQ ID NO: 1 |
| AAVhu.31 | 246 | US20150315612 SEQ ID NO: 121 |
| AAVhu.32 | 247 | US20150315612 SEQ ID NO: 2 |
| AAVhu.32 | 248 | US20150315612 SEQ ID NO: 122 |
| AAVhu.33 | 249 | US20150315612 SEQ ID NO: 75 |
| AAVhu.33 | 250 | US20150315612 SEQ ID NO: 124 |
| AAVhu.34 | 251 | US20150315612 SEQ ID NO: 72 |
| AAVhu.34 | 252 | US20150315612 SEQ ID NO: 125 |
| AAVhu.35 | 253 | US20150315612 SEQ ID NO: 73 |
| AAVhu.35 | 254 | US20150315612 SEQ ID NO: 164 |
| AAVhu.36 | 255 | US20150315612 SEQ ID NO: 74 |
| AAVhu.36 | 256 | US20150315612 SEQ ID NO: 126 |
| AAVhu.37 | 257 | US20150159173 SEQ ID NO: 34, US20150315612 |
| | | SEQ ID NO: 88 |
| AAVhu.37 (AAV106.1) | 258 | US20150315612 SEQ ID NO: 10, US20150159173 |
| | | SEQ ID NO: 18 |
| AAVhu.38 | 259 | US20150315612 SEQ ID NO: 161 |
| AAVhu.39 | 260 | US20150315612 SEQ ID NO: 102 |
| AAVhu.39 (AAVLG-9) | 261 | US20150315612 SEQ ID NO: 24 |
| AAVhu.4 | 262 | US20150315612 SEQ ID NO: 47 |
| AAVhu.4 | 263 | US20150315612 SEQ ID NO: 141 |
| AAVhu.40 | 264 | US20150315612 SEQ ID NO: 87 |
| AAVhu.40 (AAV114.3) | 265 | US20150315612 SEQ ID No: 11 |
| AAVhu.41 | 266 | US20150315612 SEQ ID NO: 91 |
| AAVhu.41 (AAV127.2) | 267 | US20150315612 SEQ ID NO: 6 |
| AAVhu.42 | 268 | US20150315612 SEQ ID NO: 85 |
| AAVhu.42 (AAV127.5) | 269 | US20150315612 SEQ ID NO: 8 |
| AAVhu.43 | 270 | US20150315612 SEQ ID NO: 160 |
| AAVhu.43 | 271 | US20150315612 SEQ ID NO: 236 |
| AAVhu.43 (AAV128.1) | 272 | US20150315612 SEQ ID NO: 80 |
| AAVhu.44 | 273 | US20150159173 SEQ ID NO: 45, US20150315612 |
| | | SEQ ID NO: 158 |
| AAVhu.44 (AAV128.3) | 274 | US20150315612 SEQ ID NO: 81 |
| AAVhu.44R1 | 275 | US20150159173 |
| AAVhu.44R2 | 276 | US20150159173 |
| AAVhu.44R3 | 277 | US20150159173 |
| AAVhu.45 | 278 | US20150315612 SEQ ID NO: 76 |
| AAVhu.45 | 279 | US20150315612 SEQ ID NO: 127 |
| AAVhu.46 | 280 | US20150315612 SEQ ID NO: 82 |
| AAVhu.46 | 281 | US20150315612 SEQ ID NO: 159 |
| AAVhu.46 | 282 | US20150315612 SEQ ID NO: 224 |
| AAVhu.47 | 283 | US20150315612 SEQ ID NO: 77 |
| AAVhu.47 | 284 | US20150315612 SEQ ID NO: 128 |
| AAVhu.48 | 285 | US20150159173 SEQ ID NO: 38 |
| AAVhu.48 | 286 | US20150315612 SEQ ID NO: 157 |
| AAVhu.48 (AAV130.4) | 287 | US20150315612 SEQ ID NO: 78 |
| AAVhu.48R1 | 288 | US20150159173 |
| AAVhu.48R2 | 289 | US20150159173 |
| AAVhu.48R3 | 290 | US20150159173 |
| AAVhu.49 | 291 | US20150315612 SEQ ID NO: 209 |
| AAVhu.49 | 292 | US20150315612 SEQ ID NO: 189 |
| AAVhu.5 | 293 | US20150315612 SEQ ID NO: 45 |
| AAVhu.5 | 294 | US20150315612 SEQ ID NO: 142 |
| AAVhu.51 | 295 | US20150315612 SEQ ID NO: 208 |
| AAVhu.51 | 296 | US20150315612 SEQ ID NO: 190 |
| AAVhu.52 | 297 | US20150315612 SEQ ID NO: 210 |
| AAVhu.52 | 298 | US20150315612 SEQ ID NO: 191 |
| AAVhu.53 | 299 | US20150159173 SEQ ID NO: 19 |
| AAVhu.53 | 300 | US20150159173 SEQ ID NO: 35 |
| AAVhu.53 (AAV145.1) | 301 | US20150315612 SEQ ID NO: 176 |
| AAVhu.54 | 302 | US20150315612 SEQ ID NO: 188 |
| AAVhu.54 (AAV145.5) | 303 | US20150315612 SEQ ID No: 177 |
| AAVhu.55 | 304 | US20150315612 SEQ ID NO: 187 |
| AAVhu.56 | 305 | US20150315612 SEQ ID NO: 205 |
| AAVhu.56 (AAV145.6) | 306 | US20150315612 SEQ ID NO: 168 |
| AAVhu.56 (AAV145.6) | 307 | US20150315612 SEQ ID NO: 192 |
| AAVhu.57 | 308 | US20150315612 SEQ ID NO: 206 |
| AAVhu.57 | 309 | US20150315612 SEQ ID NO: 169 |
| AAVhu.57 | 310 | US20150315612 SEQ ID NO: 193 |
| AAVhu.58 | 311 | US20150315612 SEQ ID NO: 207 |
| AAVhu.58 | 312 | US20150315612 SEQ ID NO: 194 |
| AAVhu.6 (AAV3.1) | 313 | US20150315612 SEQ ID NO: 5 |
| AAVhu.6 (AAV3.1) | 314 | US20150315612 SEQ ID NO: 84 |
| AAVhu.60 | 315 | US20150315612 SEQ ID NO: 184 |
| AAVhu.60 (AAV161.10) | 316 | US20150315612 SEQ ID NO: 170 |
| AAVhu.61 | 317 | US20150315612 SEQ ID NO: 185 |
| AAVhu.61 (AAV161.6) | 318 | US20150315612 SEQ ID NO: 174 |
| AAVhu.63 | 319 | US20150315612 SEQ ID NO: 204 |
| AAVhu.63 | 320 | US20150315612 SEQ ID NO: 195 |
| AAVhu.64 | 321 | US20150315612 SEQ ID NO: 212 |
| AAVhu.64 | 322 | US20150315612 SEQ ID NO: 196 |
| AAVhu.66 | 323 | US20150315612 SEQ ID NO: 197 |
| AAVhu.67 | 324 | US20150315612 SEQ ID NO: 215 |
| AAVhu.67 | 325 | US20150315612 SEQ ID NO: 198 |
| AAVhu.7 | 326 | US20150315612 SEQ ID NO: 226 |
| AAVhu.7 | 327 | US20150315612 SEQ ID NO: 150 |
| AAVhu.7 (AAV7.3) | 328 | US20150315612 SEQ ID NO: 55 |
| AAVhu.71 | 329 | US20150315612 SEQ ID NO: 79 |
| AAVhu.8 | 330 | US20150315612 SEQ ID NO: 53 |
| AAVhu.8 | 331 | US20150315612 SEQ ID NO: 12 |
| AAVhu.8 | 332 | US20150315612 SEQ ID NO: 151 |
| AAVhu.9 (AAV3.1) | 333 | US20150315612 SEQ ID NO: 58 |
| AAVhu.9 (AAV3.1) | 334 | US20150315612 SEQ ID NO: 155 |
| AAV-LK01 | 335 | US20150376607 SEQ ID NO: 2 |
| AAV-LK01 | 336 | US20150376607 SEQ ID NO: 29 |
| AAV-LK02 | 337 | US20150376607 SEQ ID NO: 3 |
| AAV-LK02 | 338 | US20150376607 SEQ ID NO: 30 |
| AAV-LK03 | 339 | US20150376607 SEQ ID NO: 4 |
| AAV-LK03 | 340 | WO2015121501 SEQ ID NO: 12, US20150376607 |
| | | SEQ ID NO: 31 |
| AAV-LK04 | 341 | US20150376607 SEQ ID NO: 5 |
| AAV-LK04 | 342 | US20150376607 SEQ ID NO: 32 |
| AAV-LK05 | 343 | US20150376607 SEQ ID NO: 6 |
| AAV-LK05 | 344 | US20150376607 SEQ ID NO: 33 |
| AAV-LK06 | 345 | US20150376607 SEQ ID NO: 7 |
| AAV-LK06 | 346 | US20150376607 SEQ ID NO: 34 |
| AAV-LK07 | 347 | US20150376607 SEQ ID NO: 8 |
| AAV-LK07 | 348 | US20150376607 SEQ ID NO: 35 |
| AAV-LK08 | 349 | US20150376607 SEQ ID NO: 9 |
| AAV-LK08 | 350 | US20150376607 SEQ ID NO: 36 |
| AAV-LK09 | 351 | US20150376607 SEQ ID NO: 10 |
| AAV-LK09 | 352 | US20150376607 SEQ ID NO: 37 |
| AAV-LK10 | 353 | US20150376607 SEQ ID NO: 11 |
| AAV-LK10 | 354 | US20150376607 SEQ ID NO: 38 |
| AAV-LK11 | 355 | US20150376607 SEQ ID NO: 12 |
| AAV-LK11 | 356 | US20150376607 SEQ ID NO: 39 |
| AAV-LK12 | 357 | US20150376607 SEQ ID NO: 13 |
| AAV-LK12 | 358 | US20150376607 SEQ ID NO: 40 |
| AAV-LK13 | 359 | US20150376607 SEQ ID NO: 14 |
| AAV-LK13 | 360 | US20150376607 SEQ ID NO: 41 |
| AAV-LK14 | 361 | US20150376607 SEQ ID NO: 15 |
| AAV-LK14 | 362 | US20150376607 SEQ ID NO: 42 |
| AAV-LK15 | 363 | US20150376607 SEQ ID NO: 16 |
| AAV-LK15 | 364 | US20150376607 SEQ ID NO: 43 |
| AAV-LK16 | 365 | US20150376607 SEQ ID NO: 17 |
| AAV-LK16 | 366 | US20150376607 SEQ ID NO: 44 |
| AAV-LK17 | 367 | US20150376607 SEQ ID NO: 18 |
| AAV-LK17 | 368 | US20150376607 SEQ ID NO: 45 |
| AAV-LK18 | 369 | US20150376607 SEQ ID NO: 19 |
| AAV-LK18 | 370 | US20150376607 SEQ ID NO: 46 |
| AAV-LK19 | 371 | US20150376607 SEQ ID NO: 20 |
| AAV-LK19 | 372 | US20150376607 SEQ ID NO: 47 |
| AAV-PAEC | 373 | US20150376607 SEQ ID NO: 1 |
| AAV-PAEC | 374 | US20150376607 SEQ ID NO: 48 |
| AAV-PAEC11 | 375 | US20150376607 SEQ ID NO: 26 |
| AAV-PAEC11 | 376 | US20150376607 SEQ ID NO: 54 |
| AAV-PAEC12 | 377 | US20150376607 SEQ ID NO: 27 |
| AAV-PAEC12 | 378 | US20150376607 SEQ ID NO: 51 |
| AAV-PAEC13 | 379 | US20150376607 SEQ ID NO: 28 |
| AAV-PAEC13 | 380 | US20150376607 SEQ ID NO: 49 |
| AAV-PAEC2 | 381 | US20150376607 SEQ ID NO: 21 |
| AAV-PAEC2 | 382 | US20150376607 SEQ ID NO: 56 |
| AAV-PAEC4 | 383 | US20150376607 SEQ ID NO: 22 |
| AAV-PAEC4 | 384 | US20150376607 SEQ ID NO: 55 |
| AAV-PAEC6 | 385 | US20150376607 SEQ ID NO: 23 |
| AAV-PAEC6 | 386 | US20150376607 SEQ ID NO: 52 |
| AAV-PAEC7 | 387 | US20150376607 SEQ ID NO: 24 |
| AAV-PAEC7 | 388 | US20150376607 SEQ ID NO: 53 |
| AAV-PAEC8 | 389 | US20150376607 SEQ ID NO: 25 |
| AAV-PAEC8 | 390 | US20150376607 SEQ ID NO: 50 |
| AAVpi.1 | 391 | US20150315612 SEQ ID NO: 28 |
| AAVpi.1 | 392 | US20150315612 SEQ ID NO: 93 |
| AAVpi.2 | 393 | US20150315612 SEQ ID NO: 30 |
| AAVpi.2 | 394 | US20150315612 SEQ ID NO: 95 |
| AAVpi.3 | 395 | US20150315612 SEQ ID NO: 29 |
| AAVpi.3 | 396 | US20150315612 SEQ ID NO: 94 |
| AAVrh.10 | 397 | US20150159173 SEQ ID NO: 9 |
| AAVrh.10 | 398 | US20150159173 SEQ ID NO: 25 |
| AAV44.2 | 399 | US20030138772 SEQ ID NO: 59 |
| AAVrh.10 (AAV44.2) | 400 | US20030138772 SEQ ID NO: 81 |
| AAV42.1B | 401 | US20030138772 SEQ ID NO: 90 |
| AAVrh.12 (AAV42.1b) | 402 | US20030138772 SEQ ID NO: 30 |
| AAVrh.13 | 403 | US20150159173 SEQ ID NO: 10 |
| AAVrh.13 | 404 | US20150159173 SEQ ID NO: 26 |
| AAVrh.13 | 405 | US20150315612 SEQ ID NO: 228 |
| AAVrh.13R | 406 | US20150159173 |
| AAV42.3A | 407 | US20030138772 SEQ ID NO: 87 |
| AAVrh.14 (AAV42.3a) | 408 | US20030138772 SEQ ID NO: 32 |
| AAV42.5A | 409 | US20030138772 SEQ ID NO: 89 |
| AAVrh.17 (AAV42.5a) | 410 | US20030138772 SEQ ID NO: 34 |
| AAV42.5B | 411 | US20030138772 SEQ ID NO: 91 |
| AAVrh.18 (AAV42.5b) | 412 | US20030138772 SEQ ID NO: 29 |
| AAV42.6B | 413 | US20030138772 SEQ ID NO: 112 |
| AAVrh.19 (AAV42.6b) | 414 | US20030138772 SEQ ID NO: 38 |
| AAVrh.2 | 415 | US20150159173 SEQ ID NO: 39 |
| AAVrh.2 | 416 | US20150315612 SEQ ID NO: 231 |
| AAVrh.20 | 417 | US20150159173 SEQ ID NO: 1 |
| AAV42.10 | 418 | US20030138772 SEQ ID NO: 106 |
| AAVrh.21 (AAV42.10) | 419 | US20030138772 SEQ ID NO: 35 |
| AAV42.11 | 420 | US20030138772 SEQ ID NO: 108 |
| AAVrh.22 (AAV42.11) | 421 | US20030138772 SEQ ID NO: 37 |
| AAV42.12 | 422 | US20030138772 SEQ ID NO: 113 |
| AAVrh.23 (AAV42.12) | 423 | US20030138772 SEQ ID NO: 58 |
| AAV42.13 | 424 | US20030138772 SEQ ID NO: 86 |
| AAVrh.24 (AAV42.13) | 425 | US20030138772 SEQ ID NO: 31 |
| AAV42.15 | 426 | US20030138772 SEQ ID NO: 84 |
| AAVrh.25 (AAV42.15) | 427 | US20030138772 SEQ ID NO: 28 |
| AAVrh.2R | 428 | US20150159173 |
| AAVrh.31 (AAV223.1) | 429 | US20030138772 SEQ ID NO: 48 |
| AAVC1 | 430 | US20030138772 SEQ ID NO: 60 |
| AAVrh.32 (AAVC1) | 431 | US20030138772 SEQ ID NO: 19 |
| AAVrh.32/33 | 432 | US20150159173 SEQ ID NO: 2 |
| AAVrh.33 (AAVC3) | 433 | US20030138772 SEQ ID NO: 20 |
| AAVC5 | 434 | US20030138772 SEQ ID NO: 62 |
| AAVrh.34 (AAVC5) | 435 | US20030138772 SEQ ID NO: 21 |
| AAVF1 | 436 | US20030138772 SEQ ID NO: 109 |
| AAVrh.35 (AAVF1) | 437 | US20030138772 SEQ ID NO: 22 |
| AAVF3 | 438 | US20030138772 SEQ ID NO: 111 |
| AAVrh.36 (AAVF3) | 439 | US20030138772 SEQ ID NO: 23 |
| AAVrh.37 | 440 | US20030138772 SEQ ID NO: 24 |
| AAVrh.37 | 441 | US20150159173 SEQ ID NO: 40 |
| AAVrh.37 | 442 | US20150315612 SEQ ID NO: 229 |
| AAVrh.37R2 | 443 | US20150159173 |
| AAVrh.38 (AAVLG-4) | 444 | US20150315612 SEQ ID NO: 7 |
| AAVrh.38 (AAVLG-4) | 445 | US20150315612 SEQ ID NO: 86 |
| AAVrh.39 | 446 | US20150159173 SEQ ID NO: 20, US20150315612 |
| | | SEQ ID NO: 13 |
| AAVrh.39 | 447 | US20150159173 SEQ ID NO: 3, US20150159173 |
| | | SEQ ID NO: 36, US20150315612 SEQ ID NO: 89 |
| AAVrh.40 | 448 | US20150315612 SEQ ID NO: 92 |
| AAVrh.40 (AAVLG-10) | 449 | US20150315612 SEQ ID No: 14 |
| AAVrh.43 (AAVN721-8) | 450 | US20150315612 SEQ ID NO: 43, US20150159173 |
| | | SEQ ID NO: 21 |
| AAVrh.43 (AAVN721-8) | 451 | US20150315612 SEQ ID NO: 163, US20150159173 |
| | | SEQ ID NO: 37 |
| AAVrh.44 | 452 | US20150315612 SEQ ID NO: 34 |
| AAVrh.44 | 453 | US20150315612 SEQ ID NO: 111 |
| AAVrh.45 | 454 | US20150315612 SEQ ID NO: 41 |
| AAVrh.45 | 455 | US20150315612 SEQ ID NO: 109 |
| AAVrh.46 | 456 | US20150159173 SEQ ID NO: 22, US20150315612 |
| | | SEQ ID NO: 19 |
| AAVrh.46 | 457 | US20150159173 SEQ ID NO: 4, US20150315612 |
| | | SEQ ID NO: 101 |
| AAVrh.47 | 458 | US20150315612 SEQ ID NO: 38 |
| AAVrh.47 | 459 | US20150315612 SEQ ID NO: 118 |
| AAVrh.48 | 460 | US20150159173 SEQ ID NO: 44, US20150315612 |
| | | SEQ ID NO: 115 |
| AAVrh.48.1 | 461 | US20150159173 |
| AAVrh.48.1.2 | 462 | US20150159173 |
| AAVrh.48.2 | 463 | US20150159173 |
| AAVrh.48 (AAV1-7) | 464 | US20150315612 SEQ ID NO: 32 |
| AAVrh.49 (AAV1-8) | 465 | US20150315612 SEQ ID NO: 25 |
| AAVrh.49 (AAV1-8) | 466 | US20150315612 SEQ ID NO: 103 |
| AAVrh.50 (AAV2-4) | 467 | US20150315612 SEQ ID NO: 23 |
| AAVrh.50 (AAV2-4) | 468 | US20150315612 SEQ ID NO: 108 |
| AAVrh.51 (AAV2-5) | 469 | US20150315612 SEQ ID No: 22 |
| AAVrh.51 (AAV2-5) | 470 | US20150315612 SEQ ID NO: 104 |
| AAVrh.52 (AAV3-9) | 471 | US20150315612 SEQ ID NO: 18 |
| AAVrh.52 (AAV3-9) | 472 | US20150315612 SEQ ID NO: 96 |
| AAVrh.53 | 473 | US20150315612 SEQ ID NO: 97 |
| AAVrh.53 (AAV3-11) | 474 | US20150315612 SEQ ID NO: 17 |
| AAVrh.53 (AAV3-11) | 475 | US20150315612 SEQ ID NO: 186 |
| AAVrh.54 | 476 | US20150315612 SEQ ID NO: 40 |
| AAVrh.54 | 477 | US20150159173 SEQ ID NO: 49, US20150315612 |
| | | SEQ ID NO: 116 |
| AAVrh.55 | 478 | US20150315612 SEQ ID NO: 37 |
| AAVrh.55 (AAV4-19) | 479 | US20150315612 SEQ ID NO: 117 |
| AAVrh.56 | 480 | US20150315612 SEQ ID NO: 54 |
| AAVrh.56 | 481 | US20150315612 SEQ ID NO: 152 |
| AAVrh.57 | 482 | US20150315612 SEQ ID NO: 26 |
| AAVrh.57 | 483 | US20150315612 SEQ ID NO: 105 |
| AAVrh.58 | 484 | US20150315612 SEQ ID NO: 27 |
| AAVrh.58 | 485 | US20150159173 SEQ ID NO: 48, US20150315612 |
| | | SEQ ID NO: 106 |
| AAVrh.58 | 486 | US20150315612 SEQ ID NO: 232 |
| AAVrh.59 | 487 | US20150315612 SEQ ID NO: 42 |
| AAVrh.59 | 488 | US20150315612 SEQ ID NO: 110 |
| AAVrh.60 | 489 | US20150315612 SEQ ID NO: 31 |
| AAVrh.60 | 490 | US20150315612 SEQ ID NO: 120 |
| AAVrh.61 | 491 | US20150315612 SEQ ID NO: 107 |
| AAVrh.61 (AAV2-3) | 492 | US20150315612 SEQ ID NO: 21 |
| AAVrh.62 (AAV2-15) | 493 | US20150315612 SEQ ID No: 33 |
| AAVrh.62 (AAV2-15) | 494 | US20150315612 SEQ ID NO: 114 |
| AAVrh.64 | 495 | US20150315612 SEQ ID No: 15 |
| AAVrh.64 | 496 | US20150159173 SEQ ID NO: 43, US20150315612 |
| | | SEQ ID NO: 99 |
| AAVrh.64 | 497 | US20150315612 SEQ ID NO: 233 |
| AAVRh.64R1 | 498 | US20150159173 |
| AAVRh.64R2 | 499 | US20150159173 |
| AAVrh.65 | 500 | US20150315612 SEQ ID NO: 35 |
| AAVrh.65 | 501 | US20150315612 SEQ ID NO: 112 |
| AAVrh.67 | 502 | US20150315612 SEQ ID NO: 36 |
| AAVrh.67 | 503 | US20150315612 SEQ ID NO: 230 |
| AAVrh.67 | 504 | US20150159173 SEQ ID NO: 47, US20150315612 |
| | | SEQ ID NO: 113 |
| AAVrh.68 | 505 | US20150315612 SEQ ID NO: 16 |
| AAVrh.68 | 506 | US20150315612 SEQ ID NO: 100 |
| AAVrh.69 | 507 | US20150315612 SEQ ID NO: 39 |
| AAVrh.69 | 508 | US20150315612 SEQ ID NO: 119 |
| AAVrh.70 | 509 | US20150315612 SEQ ID NO: 20 |
| AAVrh.70 | 510 | US20150315612 SEQ ID NO: 98 |
| AAVrh.71 | 511 | US20150315612 SEQ ID NO: 162 |
| AAVrh.72 | 512 | US20150315612 SEQ ID NO: 9 |
| AAVrh.73 | 513 | US20150159173 SEQ ID NO: 5 |
| AAVrh.74 | 514 | US20150159173 SEQ ID NO: 6 |
| AAVrh.8 | 515 | US20150159173 SEQ ID NO: 41 |
| AAVrh.8 | 516 | US20150315612 SEQ ID NO: 235 |
| AAVrh.8R | 517 | US20150159173, WO2015168666 SEQ ID NO: 9 |
| AAVrh.8R A586R mutant | 518 | WO2015168666 SEQ ID NO: 10 |
| AAVrh.8R R533A mutant | 519 | WO2015168666 SEQ ID NO: 11 |
| BAAV (bovine AAV) | 520 | US9193769 SEQ ID NO: 8 |
| BAAV (bovine AAV) | 521 | US9193769 SEQ ID NO: 10 |
| BAAV (bovine AAV) | 522 | US9193769 SEQ ID NO: 4 |
| BAAV (bovine AAV) | 523 | US9193769 SEQ ID NO: 2 |
| BAAV (bovine AAV) | 524 | US9193769 SEQ ID NO: 6 |
| BAAV (bovine AAV) | 525 | US9193769 SEQ ID NO: 1 |
| BAAV (bovine AAV) | 526 | US9193769 SEQ ID NO: 5 |
| BAAV (bovine AAV) | 527 | US9193769 SEQ ID NO: 3 |
| BAAV (bovine AAV) | 528 | US9193769 SEQ ID NO: 11 |
| BAAV (bovine AAV) | 529 | US7427396 SEQ ID NO: 5 |
| BAAV (bovine AAV) | 530 | US7427396 SEQ ID NO: 6 |
| BAAV (bovine AAV) | 531 | US9193769 SEQ ID NO: 7 |
| BAAV (bovine AAV) | 532 | US9193769 SEQ ID NO: 9 |
| BNP61 AAV | 533 | US20150238550 SEQ ID NO: 1 |
| BNP61 AAV | 534 | US20150238550 SEQ ID NO: 2 |
| BNP62 AAV | 535 | US20150238550 SEQ ID NO: 3 |
| BNP63 AAV | 536 | US20150238550 SEQ ID NO: 4 |
| caprine AAV | 537 | US7427396 SEQ ID NO: 3 |
| caprine AAV | 538 | US7427396 SEQ ID NO: 4 |
| true type AAV (ttAAV) | 539 | WO2015121501 SEQ ID NO: 2 |
| AAAV (Avian AAV) | 540 | US9238800 SEQ ID NO: 12 |
| AAAV (Avian AAV) | 541 | US9238800 SEQ ID NO: 2 |
| AAAV (Avian AAV) | 542 | US9238800 SEQ ID NO: 6 |
| AAAV (Avian AAV) | 543 | US9238800 SEQ ID NO: 4 |
| AAAV (Avian AAV) | 544 | US9238800 SEQ ID NO: 8 |
| AAAV (Avian AAV) | 545 | US9238800 SEQ ID NO: 14 |
| AAAV (Avian AAV) | 546 | US9238800 SEQ ID NO: 10 |
| AAAV (Avian AAV) | 547 | US9238800 SEQ ID NO: 15 |
| AAAV (Avian AAV) | 548 | US9238800 SEQ ID NO: 5 |
| AAAV (Avian AAV) | 549 | US9238800 SEQ ID NO: 9 |
| AAAV (Avian AAV) | 550 | US9238800 SEQ ID NO: 3 |
| AAAV (Avian AAV) | 551 | US9238800 SEQ ID NO: 7 |
| AAAV (Avian AAV) | 552 | US9238800 SEQ ID NO: 11 |
| AAAV (Avian AAV) | 553 | US9238800 SEQ ID NO: 13 |
| AAAV (Avian AAV) | 554 | US9238800 SEQ ID NO: 1 |
| AAV Shuffle 100-1 | 555 | US20160017295 SEQ ID NO: 23 |
| AAV Shuffle 100-1 | 556 | US20160017295 SEQ ID NO: 11 |
| AAV Shuffle 100-2 | 557 | US20160017295 SEQ ID NO: 37 |
| AAV Shuffle 100-2 | 558 | US20160017295 SEQ ID NO: 29 |
| AAV Shuffle 100-3 | 559 | US20160017295 SEQ ID NO: 24 |
| AAV Shuffle 100-3 | 560 | US20160017295 SEQ ID NO: 12 |
| AAV Shuffle 100-7 | 561 | US20160017295 SEQ ID NO: 25 |
| AAV Shuffle 100-7 | 562 | US20160017295 SEQ ID NO: 13 |
| AAV Shuffle 10-2 | 563 | US20160017295 SEQ ID NO: 34 |
| AAV Shuffle 10-2 | 564 | US20160017295 SEQ ID NO: 26 |
| AAV Shuffle 10-6 | 565 | US20160017295 SEQ ID NO: 35 |
| AAV Shuffle 10-6 | 566 | US20160017295 SEQ ID NO: 27 |
| AAV Shuffle 10-8 | 567 | US20160017295 SEQ ID NO: 36 |
| AAV Shuffle 10-8 | 568 | US20160017295 SEQ ID NO: 28 |
| AAV SM 100-10 | 569 | US20160017295 SEQ ID NO: 41 |
| AAV SM 100-10 | 570 | US20160017295 SEQ ID NO: 33 |
| AAV SM 100-3 | 571 | US20160017295 SEQ ID NO: 40 |
| AAV SM 100-3 | 572 | US20160017295 SEQ ID NO: 32 |
| AAV SM 10-1 | 573 | US20160017295 SEQ ID NO: 38 |
| AAV SM 10-1 | 574 | US20160017295 SEQ ID NO: 30 |
| AAV SM 10-2 | 575 | US20160017295 SEQ ID NO: 10 |
| AAV SM 10-2 | 576 | US20160017295 SEQ ID NO: 22 |
| AAV SM 10-8 | 577 | US20160017295 SEQ ID NO: 39 |
| AAV SM 10-8 | 578 | US20160017295 SEQ ID NO: 31 |
| AAVF1/HSC1 | 579 | WO2016049230 SEQ ID NO: 20 |
| AAVF2/HSC2 | 580 | WO2016049230 SEQ ID NO: 21 |
| AAVF3/HSC3 | 581 | WO2016049230 SEQ ID NO: 22 |
| AAVF4/HSC4 | 582 | WO2016049230 SEQ ID NO: 23 |
| AAVF5/HSC5 | 583 | WO2016049230 SEQ ID NO: 25 |
| AAVF6/HSC6 | 584 | WO2016049230 SEQ ID NO: 24 |
| AAVF7/HSC7 | 585 | WO2016049230 SEQ ID NO: 27 |
| AAVF8/HSC8 | 586 | WO2016049230 SEQ ID NO: 28 |
| AAVF9/HSC9 | 587 | WO2016049230 SEQ ID NO: 29 |
| AAVF11/HSC11 | 588 | WO2016049230 SEQ ID NO: 26 |
| AAVF12/HSC12 | 589 | WO2016049230 SEQ ID NO: 30 |
| AAVF13/HSC13 | 590 | WO2016049230 SEQ ID NO: 31 |
| AAVF14/HSC14 | 591 | WO2016049230 SEQ ID NO: 32 |
| AAVF15/HSC15 | 592 | WO2016049230 SEQ ID NO: 33 |
| AAVF16/HSC16 | 593 | WO2016049230 SEQ ID NO: 34 |
| AAVF17/HSC17 | 594 | WO2016049230 SEQ ID NO: 35 |
| AAVF1/HSC1 | 595 | WO2016049230 SEQ ID NO: 2 |
| AAVF2/HSC2 | 596 | WO2016049230 SEQ ID NO: 3 |
| AAVF3/HSC3 | 597 | WO2016049230 SEQ ID NO: 5 |
| AAVF4/HSC4 | 598 | WO2016049230 SEQ ID NO: 6 |
| AAVF5/HSC5 | 599 | WO2016049230 SEQ ID NO: 11 |
| AAVF6/HSC6 | 600 | WO2016049230 SEQ ID NO: 7 |
| AAVF7/HSC7 | 601 | WO2016049230 SEQ ID NO: 8 |
| AAVF8/HSC8 | 602 | WO2016049230 SEQ ID NO: 9 |
| AAVF9/HSC9 | 603 | WO2016049230 SEQ ID NO: 10 |
| AAVF11/HSC11 | 604 | WO2016049230 SEQ ID NO: 4 |
| AAVF12/HSC12 | 605 | WO2016049230 SEQ ID NO: 12 |
| AAVF13/HSC13 | 606 | WO2016049230 SEQ ID NO: 14 |
| AAVF14/HSC14 | 607 | WO2016049230 SEQ ID NO: 15 |
| AAVF15/HSC15 | 608 | WO2016049230 SEQ ID NO: 16 |
| AAVF16/HSC16 | 609 | WO2016049230 SEQ ID NO: 17 |
| AAVF17/HSC17 | 610 | WO2016049230 SEQ ID NO: 13 |
| AAV CBr-E1 | 611 | US8734809 SEQ ID NO: 13 |
| AAV CBr-E2 | 612 | US8734809 SEQ ID NO: 14 |
| AAV CBr-E3 | 613 | US8734809 SEQ ID NO: 15 |
| AAV CBr-E4 | 614 | US8734809 SEQ ID NO: 16 |
| AAV CBr-E5 | 615 | US8734809 SEQ ID NO: 17 |
| AAV CBr-e5 | 616 | US8734809 SEQ ID NO: 18 |
| AAV CBr-E6 | 617 | US8734809 SEQ ID NO: 19 |
| AAV CBr-E7 | 618 | US8734809 SEQ ID NO: 20 |
| AAV CBr-E8 | 619 | US8734809 SEQ ID NO: 21 |
| AAV CLv-D1 | 620 | US8734809 SEQ ID NO: 22 |
| AAV CLv-D2 | 621 | US8734809 SEQ ID NO: 23 |
| AAV CLv-D3 | 622 | US8734809 SEQ ID NO: 24 |
| AAV CLv-D4 | 623 | US8734809 SEQ ID NO: 25 |
| AAV CLv-D5 | 624 | US8734809 SEQ ID NO: 26 |
| AAV CLv-D6 | 625 | US8734809 SEQ ID NO: 27 |
| AAV CLv-D7 | 626 | US8734809 SEQ ID NO: 28 |
| AAV CLv-D8 | 627 | US8734809 SEQ ID NO: 29 |
| AAV CLv-E1 | 628 | US8734809 SEQ ID NO: 13 |
| AAV CLv-R1 | 629 | US8734809 SEQ ID NO: 30 |
| AAV CLv-R2 | 630 | US8734809 SEQ ID NO: 31 |
| AAV CLv-R3 | 631 | US8734809 SEQ ID NO: 32 |
| AAV CLv-R4 | 632 | US8734809 SEQ ID NO: 33 |
| AAV CLv-R5 | 633 | US8734809 SEQ ID NO: 34 |
| AAV CLv-R6 | 634 | US8734809 SEQ ID NO: 35 |
| AAV CLv-R7 | 635 | US8734809 SEQ ID NO: 36 |
| AAV CLv-R8 | 636 | US8734809 SEQ ID NO: 37 |
| AAV CLv-R9 | 637 | US8734809 SEQ ID NO: 38 |
| AAV CLg-F1 | 638 | US8734809 SEQ ID NO: 39 |
| AAV CLg-F2 | 639 | US8734809 SEQ ID NO: 40 |
| AAV CLg-F3 | 640 | US8734809 SEQ ID NO: 41 |
| AAV CLg-F4 | 641 | US8734809 SEQ ID NO: 42 |
| AAV CLg-F5 | 642 | US8734809 SEQ ID NO: 43 |
| AAV CLg-F6 | 643 | US8734809 SEQ ID NO: 43 |
| AAV CLg-F7 | 644 | US8734809 SEQ ID NO: 44 |
| AAV CLg-F8 | 645 | US8734809 SEQ ID NO: 43 |
| AAV CSp-1 | 646 | US8734809 SEQ ID NO: 45 |
| AAV CSp-10 | 647 | US8734809 SEQ ID NO: 46 |
| AAV CSp-11 | 648 | US8734809 SEQ ID NO: 47 |
| AAV CSp-2 | 649 | US8734809 SEQ ID NO: 48 |
| AAV CSp-3 | 650 | US8734809 SEQ ID NO: 49 |
| AAV CSp-4 | 651 | US8734809 SEQ ID NO: 50 |
| AAV CSp-6 | 652 | US8734809 SEQ ID NO: 51 |
| AAV CSp-7 | 653 | US8734809 SEQ ID NO: 52 |
| AAV CSp-8 | 654 | US8734809 SEQ ID NO: 53 |
| AAV CSp-9 | 655 | US8734809 SEQ ID NO: 54 |
| AAV CHt-2 | 656 | US8734809 SEQ ID NO: 55 |
| AAV CHt-3 | 657 | US8734809 SEQ ID NO: 56 |
| AAV CKd-1 | 658 | US8734809 SEQ ID NO: 57 |
| AAV CKd-10 | 659 | US8734809 SEQ ID NO: 58 |
| AAV CKd-2 | 660 | US8734809 SEQ ID NO: 59 |
| AAV CKd-3 | 661 | US8734809 SEQ ID NO: 60 |
| AAV CKd-4 | 662 | US8734809 SEQ ID NO: 61 |
| AAV CKd-6 | 663 | US8734809 SEQ ID NO: 62 |
| AAV CKd-7 | 664 | US8734809 SEQ ID NO: 63 |
| AAV CKd-8 | 665 | US8734809 SEQ ID NO: 64 |
| AAV CLv-1 | 666 | US8734809 SEQ ID NO: 65 |
| AAV CLv-12 | 667 | US8734809 SEQ ID NO: 66 |
| AAV CLv-13 | 668 | US8734809 SEQ ID NO: 67 |
| AAV CLv-2 | 669 | US8734809 SEQ ID NO: 68 |
| AAV CLv-3 | 670 | US8734809 SEQ ID NO: 69 |
| AAV CLv-4 | 671 | US8734809 SEQ ID NO: 70 |
| AAV CLv-6 | 672 | US8734809 SEQ ID NO: 71 |
| AAV CLv-8 | 673 | US8734809 SEQ ID NO: 72 |
| AAV CKd-B1 | 674 | US8734809 SEQ ID NO: 73 |
| AAV CKd-B2 | 675 | US8734809 SEQ ID NO: 74 |
| AAV CKd-B3 | 676 | US8734809 SEQ ID NO: 75 |
| AAV CKd-B4 | 677 | US8734809 SEQ ID NO: 76 |
| AAV CKd-B5 | 678 | US8734809 SEQ ID NO: 77 |
| AAV CKd-B6 | 679 | US8734809 SEQ ID NO: 78 |
| AAV CKd-B7 | 680 | US8734809 SEQ ID NO: 79 |
| AAV CKd-B8 | 681 | US8734809 SEQ ID NO: 80 |
| AAV CKd-H1 | 682 | US8734809 SEQ ID NO: 81 |
| AAV CKd-H2 | 683 | US8734809 SEQ ID NO: 82 |
| AAV CKd-H3 | 684 | US8734809 SEQ ID NO: 83 |
| AAV CKd-H4 | 685 | US8734809 SEQ ID NO: 84 |
| AAV CKd-H5 | 686 | US8734809 SEQ ID NO: 85 |
| AAV CKd-H6 | 687 | US8734809 SEQ ID NO: 77 |
| AAV CHt-1 | 688 | US8734809 SEQ ID NO: 86 |
| AAV CLv1-1 | 689 | US8734809 SEQ ID NO: 171 |
| AAV CLv1-2 | 690 | US8734809 SEQ ID NO: 172 |
| AAV CLv1-3 | 691 | US8734809 SEQ ID NO: 173 |
| AAV CLv1-4 | 692 | US8734809 SEQ ID NO: 174 |
| AAV Clv1-7 | 693 | US8734809 SEQ ID NO: 175 |
| AAV Clv-8 | 694 | US8734809 SEQ ID NO: 176 |
| AAV Clv1-9 | 695 | US8734809 SEQ ID NO: 177 |
| AAV Clv1-10 | 696 | US8734809 SEQ ID NO: 178 |
| AAV.VR-355 | 697 | US8734809 SEQ ID NO: 181 |
| AAV.hu.48R3 | 698 | US8734809 SEQ ID NO: 183 |
| AAV CBr-E1 | 699 | US8734809 SEQ ID NO: 87 |
| AAV CBr-E2 | 700 | US8734809 SEQ ID NO: 88 |
| AAV CBr-E3 | 701 | US8734809 SEQ ID NO: 89 |
| AAV CBr-E4 | 702 | US8734809 SEQ ID NO: 90 |
| AAV CBr-E5 | 703 | US8734809 SEQ ID NO: 91 |
| AAV CBr-e5 | 704 | US8734809 SEQ ID NO: 92 |
| AAV CBr-E6 | 705 | US8734809 SEQ ID NO: 93 |
| AAV CBr-E7 | 706 | US8734809 SEQ ID NO: 94 |
| AAV CBr-E8 | 707 | US8734809 SEQ ID NO: 95 |
| AAV CLv-D1 | 708 | US8734809 SEQ ID NO: 96 |
| AAV CLv-D2 | 709 | US8734809 SEQ ID NO: 97 |
| AAV CLv-D3 | 710 | US8734809 SEQ ID NO: 98 |
| AAV CLv-D4 | 711 | US8734809 SEQ ID NO: 99 |
| AAV CLv-D5 | 712 | US8734809 SEQ ID NO: 100 |
| AAV CLv-D6 | 713 | US8734809 SEQ ID NO: 101 |
| AAV CLv-D7 | 714 | US8734809 SEQ ID NO: 102 |
| AAV CLv-D8 | 715 | US8734809 SEQ ID NO: 103 |
| AAV CLv-E1 | 716 | US8734809 SEQ ID NO: 87 |
| AAV CLv-R1 | 717 | US8734809 SEQ ID NO: 104 |
| AAV CLv-R2 | 718 | US8734809 SEQ ID NO: 105 |
| AAV CLv-R3 | 719 | US8734809 SEQ ID NO: 106 |
| AAV CLv-R4 | 720 | US8734809 SEQ ID NO: 107 |
| AAV CLv-R5 | 721 | US8734809 SEQ ID NO: 108 |
| AAV CLv-R6 | 722 | US8734809 SEQ ID NO: 109 |
| AAV CLv-R7 | 723 | US8734809 SEQ ID NO: 110 |
| AAV CLv-R8 | 724 | US8734809 SEQ ID NO: 111 |
| AAV CLv-R9 | 725 | US8734809 SEQ ID NO: 112 |
| AAV CLg-F1 | 726 | US8734809 SEQ ID NO: 113 |
| AAV CLg-F2 | 727 | US8734809 SEQ ID NO: 114 |
| AAV CLg-F3 | 728 | US8734809 SEQ ID NO: 115 |
| AAV CLg-F4 | 729 | US8734809 SEQ ID NO: 116 |
| AAV CLg-F5 | 730 | US8734809 SEQ ID NO: 117 |
| AAV CLg-F6 | 731 | US8734809 SEQ ID NO: 117 |
| AAV CLg-F7 | 732 | US8734809 SEQ ID NO: 118 |
| AAV CLg-F8 | 733 | US8734809 SEQ ID NO: 117 |
| AAV CSp-1 | 734 | US8734809 SEQ ID NO: 119 |
| AAV CSp-10 | 735 | US8734809 SEQ ID NO: 120 |
| AAV CSp-11 | 736 | US8734809 SEQ ID NO: 121 |
| AAV CSp-2 | 737 | US8734809 SEQ ID NO: 122 |
| AAV CSp-3 | 738 | US8734809 SEQ ID NO: 123 |
| AAV CSp-4 | 739 | US8734809 SEQ ID NO: 124 |
| AAV CSp-6 | 740 | US8734809 SEQ ID NO: 125 |
| AAV CSp-7 | 741 | US8734809 SEQ ID NO: 126 |
| AAV CSp-8 | 742 | US8734809 SEQ ID NO: 127 |
| AAV CSp-9 | 743 | US8734809 SEQ ID NO: 128 |
| AAV CHt-2 | 744 | US8734809 SEQ ID NO: 129 |
| AAV CHt-3 | 745 | US8734809 SEQ ID NO: 130 |
| AAV CKd-1 | 746 | US8734809 SEQ ID NO: 131 |
| AAV CKd-10 | 747 | US8734809 SEQ ID NO: 132 |
| AAV CKd-2 | 748 | US8734809 SEQ ID NO: 133 |
| AAV CKd-3 | 749 | US8734809 SEQ ID NO: 134 |
| AAV CKd-4 | 750 | US8734809 SEQ ID NO: 135 |
| AAV CKd-6 | 751 | US8734809 SEQ ID NO: 136 |
| AAV CKd-7 | 752 | US8734809 SEQ ID NO: 137 |
| AAV CKd-8 | 753 | US8734809 SEQ ID NO: 138 |
| AAV CLv-1 | 754 | US8734809 SEQ ID NO: 139 |
| AAV CLv-12 | 755 | US8734809 SEQ ID NO: 140 |
| AAV CLv-13 | 756 | US8734809 SEQ ID NO: 141 |
| AAV CLv-2 | 757 | US8734809 SEQ ID NO: 142 |
| AAV CLv-3 | 758 | US8734809 SEQ ID NO: 143 |
| AAV CLv-4 | 759 | US8734809 SEQ ID NO: 144 |
| AAV CLv-6 | 760 | US8734809 SEQ ID NO: 145 |
| AAV CLv-8 | 761 | US8734809 SEQ ID NO: 146 |
| AAV CKd-B1 | 762 | US8734809 SEQ ID NO: 147 |
| AAV CKd-B2 | 763 | US8734809 SEQ ID NO: 148 |
| AAV CKd-B3 | 764 | US8734809 SEQ ID NO: 149 |
| AAV CKd-B4 | 765 | US8734809 SEQ ID NO: 150 |
| AAV CKd-B5 | 766 | US8734809 SEQ ID NO: 151 |
| AAV CKd-B6 | 767 | US8734809 SEQ ID NO: 152 |
| AAV CKd-B7 | 768 | US8734809 SEQ ID NO: 153 |
| AAV CKd-B8 | 769 | US8734809 SEQ ID NO: 154 |
| AAV CKd-H1 | 770 | US8734809 SEQ ID NO: 155 |
| AAV CKd-H2 | 771 | US8734809 SEQ ID NO: 156 |
| AAV CKd-H3 | 772 | US8734809 SEQ ID NO: 157 |
| AAV CKd-H4 | 773 | US8734809 SEQ ID NO: 158 |
| AAV CKd-H5 | 774 | US8734809 SEQ ID NO: 159 |
| AAV CKd-H6 | 775 | US8734809 SEQ ID NO: 151 |
| AAV CHt-1 | 776 | US8734809 SEQ ID NO: 160 |
| AAV CHt-P2 | 777 | WO2016065001 SEQ ID NO: 1 |
| AAV CHt-P5 | 778 | WO2016065001 SEQ ID NO: 2 |
| AAV CHt-P9 | 779 | WO2016065001 SEQ ID NO: 3 |
| AAV CBr-7.1 | 780 | WO2016065001 SEQ ID NO: 4 |
| AAV CBr-7.2 | 781 | WO2016065001 SEQ ID NO: 5 |
| AAV CBr-7.3 | 782 | WO2016065001 SEQ ID NO: 6 |
| AAV CBr-7.4 | 783 | WO2016065001 SEQ ID NO: 7 |
| AAV CBr-7.5 | 784 | WO2016065001 SEQ ID NO: 8 |
| AAV CBr-7.7 | 785 | WO2016065001 SEQ ID NO: 9 |
| AAV CBr-7.8 | 786 | WO2016065001 SEQ ID NO: 10 |
| AAV CBr-7.10 | 787 | WO2016065001 SEQ ID NO: 11 |
| AAV CKd-N3 | 788 | WO2016065001 SEQ ID NO: 12 |
| AAV CKd-N4 | 789 | WO2016065001 SEQ ID NO: 13 |
| AAV CKd-N9 | 790 | WO2016065001 SEQ ID NO: 14 |
| AAV CLv-L4 | 791 | WO2016065001 SEQ ID NO: 15 |
| AAV CLv-L5 | 792 | WO2016065001 SEQ ID NO: 16 |
| AAV CLv-L6 | 793 | WO2016065001 SEQ ID NO: 17 |
| AAV CLv-K1 | 794 | WO2016065001 SEQ ID NO: 18 |
| AAV CLv-K3 | 795 | WO2016065001 SEQ ID NO: 19 |
| AAV CLv-K6 | 796 | WO2016065001 SEQ ID NO: 20 |
| AAV CLv-M1 | 797 | WO2016065001 SEQ ID NO: 21 |
| AAV CLv-M11 | 798 | WO2016065001 SEQ ID NO: 22 |
| AAV CLv-M2 | 799 | WO2016065001 SEQ ID NO: 23 |
| AAV CLv-M5 | 800 | WO2016065001 SEQ ID NO: 24 |
| AAV CLv-M6 | 801 | WO2016065001 SEQ ID NO: 25 |
| AAV CLv-M7 | 802 | WO2016065001 SEQ ID NO: 26 |
| AAV CLv-M8 | 803 | WO2016065001 SEQ ID NO: 27 |
| AAV CLv-M9 | 804 | WO2016065001 SEQ ID NO: 28 |
| AAV CHt-P1 | 805 | WO2016065001 SEQ ID NO: 29 |
| AAV CHt-P6 | 806 | WO2016065001 SEQ ID NO: 30 |
| AAV CHt-P8 | 807 | WO2016065001 SEQ ID NO: 31 |
| AAV CHt-6.1 | 808 | WO2016065001 SEQ ID NO: 32 |
| AAV CHt6.10 | 809 | WO2016065001 SEQ ID NO: 33 |
| AAV CHt-6.5 | 810 | WO2016065001 SEQ ID NO: 34 |
| AAV CHt-6.6 | 811 | WO2016065001 SEQ ID NO: 35 |
| AAV CHt-6.7 | 812 | WO2016065001 SEQ ID NO: 36 |
| AAV CHt-6.8 | 813 | WO2016065001 SEQ ID NO: 37 |
| AAV CSp-8.10 | 814 | WO2016065001 SEQ ID NO: 38 |
| AAV CSp-8.2 | 815 | WO2016065001 SEQ ID NO: 39 |
| AAV CSp-8.4 | 816 | WO2016065001 SEQ ID NO: 40 |
| AAV CSp-8.5 | 817 | WO2016065001 SEQ ID NO: 41 |
| AAV CSp-8.6 | 818 | WO2016065001 SEQ ID NO: 42 |
| AAV CSp-8.7 | 819 | WO2016065001 SEQ ID NO: 43 |
| AAV CSp-8.8 | 870 | WO2016065001 SEQ ID NO: 44 |
| AAV CSp-8.9 | 821 | WO2016065001 SEQ ID NO: 45 |
| AAV CBr-B7.3 | 822 | WO2016065001 SEQ ID NO: 46 |
| AAV CBr-B7.4 | 823 | WO2016065001 SEQ ID NO: 47 |
| AAV3B | 824 | WO2016065001 SEQ ID NO: 48 |
| AAV4 | 825 | WO2016065001 SEQ ID NO: 49 |
| AAV5 | 826 | WO2016065001 SEQ ID NO: 50 |
| AAV CHt-P2 | 827 | WO2016065001 SEQ ID NO: 51 |
| AAV CHt-P5 | 828 | WO2016065001 SEQ ID NO: 52 |
| AAV CHt-P9 | 829 | WO2016065001 SEQ ID NO: 53 |
| AAV CBr-7.1 | 830 | WO2016065001 SEQ ID NO: 54 |
| AAV CBr-7.2 | 831 | WO2016065001 SEQ ID NO: 55 |
| AAV CBr-7.3 | 832 | WO2016065001 SEQ ID NO: 56 |
| AAV CBr-7.4 | 833 | WO2016065001 SEQ ID NO: 57 |
| AAV CBr-7.5 | 834 | WO2016065001 SEQ ID NO: 58 |
| AAV CBr-7.7 | 835 | WO2016065001 SEQ ID NO: 59 |
| AAV CBr-7.8 | 836 | WO2016065001 SEQ ID NO: 60 |
| AAV CBr-7.10 | 837 | WO2016065001 SEQ ID NO: 61 |
| AAV CKd-N3 | 838 | WO2016065001 SEQ ID NO: 62 |
| AAV CKd-N4 | 839 | WO2016065001 SEQ ID NO: 63 |
| AAV CKd-N9 | 840 | WO2016065001 SEQ ID NO: 64 |
| AAV CLv-L4 | 841 | WO2016065001 SEQ ID NO: 65 |
| AAV CLv-L5 | 842 | WO2016065001 SEQ ID NO: 66 |
| AAV CLv-L6 | 843 | WO2016065001 SEQ ID NO: 67 |
| AAV CLv-K1 | 844 | WO2016065001 SEQ ID NO: 68 |
| AAV CLv-K3 | 845 | WO2016065001 SEQ ID NO: 69 |
| AAV CLv-K6 | 846 | WO2016065001 SEQ ID NO: 70 |
| AAV CLv-M1 | 847 | WO2016065001 SEQ ID NO: 71 |
| AAV CLv-M11 | 848 | WO2016065001 SEQ ID NO: 72 |
| AAV CLv-M2 | 849 | WO2016065001 SEQ ID NO: 73 |
| AAV CLv-M5 | 850 | WO2016065001 SEQ ID NO: 74 |
| AAV CLv-M6 | 851 | WO2016065001 SEQ ID NO: 75 |
| AAV CLv-M7 | 852 | WO2016065001 SEQ ID NO: 76 |
| AAV CLv-M8 | 853 | WO2016065001 SEQ ID NO: 77 |
| AAV CLv-M9 | 854 | WO2016065001 SEQ ID NO: 78 |
| AAV CHt-P1 | 855 | WO2016065001 SEQ ID NO: 79 |
| AAV CHt-P6 | 856 | WO2016065001 SEQ ID NO: 80 |
| AAV CHt-P8 | 857 | WO2016065001 SEQ ID NO: 81 |
| AAV CHt-6.1 | 858 | WO2016065001 SEQ ID NO: 82 |
| AAV CHt-6.10 | 859 | WO2016065001 SEQ ID NO: 83 |
| AAV CHt-6.5 | 860 | WO2016065001 SEQ ID NO: 84 |
| AAV CHt-6.6 | 861 | WO2016065001 SEQ ID NO: 85 |
| AAV CHt-6.7 | 862 | WO2016065001 SEQ ID NO: 86 |
| AAV CHt-6.8 | 863 | WO2016065001 SEQ ID NO: 87 |
| AAV CSp-8.10 | 864 | WO2016065001 SEQ ID NO: 88 |
| AAV CSp-8.2 | 865 | WO2016065001 SEQ ID NO: 89 |
| AAV CSp-8.4 | 866 | WO2016065001 SEQ ID NO: 90 |
| AAV CSp-8.5 | 867 | WO2016065001 SEQ ID NO: 91 |
| AAV CSp-8.6 | 868 | WO2016065001 SEQ ID NO: 92 |
| AAV CSp-8.7 | 869 | WO2016065001 SEQ ID NO: 93 |
| AAV CSp-8.8 | 870 | WO2016065001 SEQ ID NO: 94 |
| AAV CSp-8.9 | 871 | WO2016065001 SEQ ID NO: 95 |
| AAV CBr-B7.3 | 872 | WO2016065001 SEQ ID NO: 96 |
| AAV CBr-B7.4 | 873 | WO2016065001 SEQ ID NO: 97 |
| AAV3B | 874 | WO2016065001 SEQ ID NO: 98 |
| AAV4 | 875 | WO2016065001 SEQ ID NO: 99 |
| AAV5 | 876 | WO2016065001 SEQ ID NO: 100 |
| GPV | 877 | US9624274B2 SEQ ID NO: 192 |
| B19 | 878 | US9624274B2 SEQ ID NO: 193 |
| MVM | 879 | US9624274B2 SEQ ID NO: 194 |
| FPV | 880 | US9624274B2 SEQ ID NO: 195 |
| CPV | 881 | US9624274B2 SEQ ID NO: 196 |
| AAV6 | 882 | US9546112B2 SEQ ID NO: 5 |
| AAV6 | 883 | US9457103B2 SEQ ID NO: 1 |
| AAV2 | 884 | US9457103B2 SEQ ID NO: 2 |
| ShH10 | 885 | US9457103B2 SEQ ID NO: 3 |
| ShH13 | 886 | US9457103B2 SEQ ID NO: 4 |
| ShH10 | 887 | US9457103B2 SEQ ID NO: 5 |
| ShH10 | 888 | US9457103B2 SEQ ID NO: 6 |
| ShH10 | 889 | US9457103B2 SEQ ID NO: 7 |
| ShH10 | 890 | US9457103B2 SEQ ID NO: 8 |
| ShH10 | 891 | US9457103B2 SEQ ID NO: 9 |
| rh74 | 892 | US9434928B2 SEQ ID NO: 1, US2015023924A1 |
| | | SEQ ID NO: 2 |
| rh74 | 893 | US9434928B2 SEQ ID NO: 2, US2015023924A1 |
| | | SEQ ID NO: 1 |
| AAV8 | 894 | US9434928B2 SEQ ID NO: 4 |
| rh74 | 895 | US9434928B2 SEQ ID NO: 5 |
| rh74 (RHM4-1) | 896 | US2015023924A1 SEQ ID NO: 5, US20160375110A1 |
| | | SEQ ID NO: 4 |
| rh74 (RHM15-1) | 897 | US2015023924A1 SEQ ID NO: 6, US20160375110A1 |
| | | SEQ ID NO: 5 |
| rh74 (RHM15-2) | 898 | US2015023924A1 SEQ ID NO: 7, US20160375110A1 |
| | | SEQ ID NO: 6 |
| rh74 (RHM15-3/ | 899 | US2015023924A1 SEQ ID NO: 8, US20160375110A1 |
| RHM15-5) | | SEQ ID NO: 7 |
| rh74 (RHM15-4) | 900 | US2015023924A1 SEQ ID NO: 9, US20160375110A1 |
| | | SEQ ID NO: 8 |
| rh74 (RHM15-6) | 901 | US2015023924A1 SEQ ID NO: 10, US20160375110A1 |
| | | SEQ ID NO: 9 |
| rh74 (RHM4-1) | 902 | US2015023924A1 SEQ ID NO: 11 |
| rh74 (RHM15-1) | 903 | US2015023924A1 SEQ ID NO: 12 |
| rh74 (RHM15-2) | 904 | US2015023924A1 SEQ ID NO: 13 |
| rh74 (RHM15-3/ | 905 | US2015023924A1 SEQ ID NO: 14 |
| RHM15-5) | | |
| rh74 (RHM15-4) | 906 | US2015023924A1 SEQ ID NO: 15 |
| rh74 (RHM15-6) | 907 | US2015023924A1 SEQ ID NO: 16 |
| AAV2 (comprising lung | 908 | US20160175389A1 SEQ ID NO: 9 |
| specific polypeptide) | | |
| AAV2 (comprising lung | 909 | US20160175389A1 SEQ ID NO: 10 |
| specific polypeptide) | | |
| Anc80 | 910 | US20170051257A1 SEQ ID NO: 1 |
| Anc80 | 911 | US20170051257A1 SEQ ID NO: 2 |
| Anc81 | 912 | US20170051257A1 SEQ ID NO: 3 |
| Anc80 | 913 | US20170051257A1 SEQ ID NO: 4 |
| Anc82 | 914 | US20170051257A1 SEQ ID NO: 5 |
| Anc82 | 915 | US20170051257A1 SEQ ID NO: 6 |
| Anc83 | 916 | US20170051257A1 SEQ ID NO: 7 |
| Anc83 | 917 | US20170051257A1 SEQ ID NO: 8 |
| Anc84 | 918 | US20170051257A1 SEQ ID NO: 9 |
| Anc84 | 919 | US20170051257A1 SEQ ID NO: 10 |
| Anc94 | 920 | US20170051257A1 SEQ ID NO: 11 |
| Anc94 | 921 | US20170051257A1 SEQ ID NO: 12 |
| Anc113 | 922 | US20170051257A1 SEQ ID NO: 13 |
| Anc113 | 923 | US20170051257A1 SEQ ID NO: 14 |
| Anc126 | 924 | US20170051257A1 SEQ ID NO: 15 |
| Anc126 | 925 | US20170051257A1 SEQ ID NO: 16 |
| Anc127 | 926 | US20170051257A1 SEQ ID NO: 17 |
| Anc127 | 927 | US20170051257A1 SEQ ID NO: 18 |
| Anc80L27 | 928 | US20170051257A1 SEQ ID NO: 19 |
| Anc80L59 | 929 | US20170051257A1 SEQ ID NO: 20 |
| Anc80L60 | 930 | US20170051257A1 SEQ ID NO: 21 |
| Anc80L62 | 931 | US20170051257A1 SEQ ID NO: 22 |
| Anc80L65 | 932 | US20170051257A1 SEQ ID NO: 23 |
| Anc80L33 | 933 | US20170051257A1 SEQ ID NO: 24 |
| Anc80L36 | 934 | US20170051257A1 SEQ ID NO: 25 |
| Anc80L44 | 935 | US20170051257A1 SEQ ID NO: 26 |
| Anc80L1 | 936 | US20170051257A1 SEQ ID NO: 35 |
| Anc80L1 | 937 | US20170051257A1 SEQ ID NO: 36 |
| AAV-X1 | 938 | US8283151B2 SEQ ID NO: 11 |
| AAV-X1b | 939 | US8283151B2 SEQ ID NO: 12 |
| AAV-X5 | 940 | US8283151B2 SEQ ID NO: 13 |
| AAV-X19 | 941 | US8283151B2 SEQ ID NO: 14 |
| AAV-X21 | 942 | US8283151B2 SEQ ID NO: 15 |
| AAV-X22 | 943 | US8283151B2 SEQ ID NO: 16 |
| AAV-X23 | 944 | US8283151B2 SEQ ID NO: 17 |
| AAV-X24 | 945 | US8283151B2 SEQ ID NO: 18 |
| AAV-X25 | 946 | US8283151B2 SEQ ID NO: 19 |
| AAV-X26 | 947 | US8283151B2 SEQ ID NO: 20 |
| AAV-X1 | 948 | US8283151B2 SEQ ID NO: 21 |
| AAV-X1b | 949 | US8283151B2 SEQ ID NO: 22 |
| AAV-X5 | 950 | US8283151B2 SEQ ID NO: 23 |
| AAV-X19 | 951 | US8283151B2 SEQ ID NO: 24 |
| AAV-X21 | 952 | US8283151B2 SEQ ID NO: 25 |
| AAV-X22 | 953 | US8283151B2 SEQ ID NO: 26 |
| AAV-X23 | 954 | US8283151B2 SEQ ID NO: 27 |
| AAV-X24 | 955 | US8283151B2 SEQ ID NO: 28 |
| AAV-X25 | 956 | US8283151B2 SEQ ID NO: 29 |
| AAV-X26 | 957 | US8283151B2 SEQ ID NO: 30 |
| AAVrh8 | 958 | WO2016054554A1 SEQ ID NO: 8 |
| AAVrh8VP2FC5 | 959 | WO2016054554A1 SEQ ID NO: 9 |
| AAVrh8VP2FC44 | 960 | WO2016054554A1 SEQ ID NO: 10 |
| AAVrh8VP2ApoB100 | 961 | WO2016054554A1 SEQ ID NO: 11 |
| AAVrh8VP2RVG | 962 | WO2016054554A1 SEQ ID NO: 12 |
| AAVrh8VP2Angiopep-2 | 963 | WO2016054554A1 SEQ ID NO: 13 |
| VP2 | | |
| AAV9.47VP1.3 | 964 | WO2016054554A1 SEQ ID NO: 14 |
| AAV9.47VP2ICAMg3 | 965 | WO2016054554A1 SEQ ID NO: 15 |
| AAV9.47VP2RVG | 966 | WO2016054554A1 SEQ ID NO: 16 |
| AAV9.47VP2Angiopep-2 | 967 | WO2016054554A1 SEQ ID NO: 17 |
| AAV9.47VP2A-string | 968 | WO2016054554A1 SEQ ID NO: 18 |
| AAVrh8VP2FC5 VP2 | 969 | WO2016054554A1 SEQ ID NO: 19 |
| AAVrh8VP2FC44 VP2 | 970 | WO2016054554A1 SEQ ID NO: 20 |
| AAVrh8VP2ApoB100 VP2 | 971 | WO2016054554A1 SEQ ID NO: 21 |
| AAVrh8VP2RVG VP2 | 972 | WO2016054554A1 SEQ ID NO: 22 |
| AAVrh8VP2Angiopep-2 | 973 | WO2016054554A1 SEQ ID NO: 23 |
| VP2 | | |
| AAV9.47VP2ICAMg3 VP2 | 974 | WO2016054554A1 SEQ ID NO: 24 |
| AAV9.47VP2RVG VP2 | 975 | WO2016054554A1 SEQ ID NO: 25 |
| AAV9.47VP2Angiopep-2 | 976 | WO2016054554A1 SEQ ID NO: 26 |
| VP2 | | |
| AAV9.47VP2A-string VP2 | 977 | WO2016054554A1 SEQ ID NO: 27 |
| rAAV-B1 | 978 | WO2016054557A1 SEQ ID NO: 1 |
| rAAV-B2 | 979 | WO2016054557A1 SEQ ID NO: 2 |
| rAAV-B3 | 980 | WO2016054557A1 SEQ ID NO: 3 |
| rAAV-B4 | 981 | WO2016054557A1 SEQ ID NO: 4 |
| rAAV-B1 | 982 | WO2016054557A1 SEQ ID NO: 5 |
| rAAV-B2 | 983 | WO2016054557A1 SEQ ID NO: 6 |
| rAAV-B3 | 984 | WO2016054557A1 SEQ ID NO: 7 |
| rAAV-B4 | 985 | WO2016054557A1 SEQ ID NO: 8 |
| rAAV-L1 | 986 | WO2016054557A1 SEQ ID NO: 9 |
| rAAV-L2 | 987 | WO2016054557A1 SEQ ID NO: 10 |
| rAAV-L3 | 988 | WO2016054557A1 SEQ ID NO: 11 |
| rAAV-L4 | 989 | WO2016054557A1 SEQ ID NO: 12 |
| rAAV-L1 | 990 | WO2016054557A1 SEQ ID NO: 13 |
| rAAV-L2 | 991 | WO2016054557A1 SEQ ID NO: 14 |
| rAAV-L3 | 992 | WO2016054557A1 SEQ ID NO: 15 |
| rAAV-L4 | 993 | WO2016054557A1 SEQ ID NO: 16 |
| AAV9 | 994 | WO2016073739A1 SEQ ID NO: 3 |
| rAAV | 995 | WO2016081811A1 SEQ ID NO: 1 |
| rAAV | 996 | WO2016081811A1 SEQ ID NO: 2 |
| rAAV | 997 | WO2016081811A1 SEQ ID NO: 3 |
| rAAV | 998 | WO2016081811A1 SEQ ID NO: 4 |
| rAAV | 999 | WO2016081811A1 SEQ ID NO: 5 |
| rAAV | 1000 | WO2016081811A1 SEQ ID NO: 6 |
| rAAV | 1001 | WO2016081811A1 SEQ ID NO: 7 |
| rAAV | 1002 | WO2016081811A1 SEQ ID NO: 8 |
| rAAV | 1003 | WO2016081811A1 SEQ ID NO: 9 |
| rAAV | 1004 | WO2016081811A1 SEQ ID NO: 10 |
| rAAV | 1005 | WO2016081811A1 SEQ ID NO: 11 |
| rAAV | 1006 | WO2016081811A1 SEQ ID NO: 12 |
| rAAV | 1007 | WO2016081811A1 SEQ ID NO: 13 |
| rAAV | 1008 | WO2016081811A1 SEQ ID NO: 14 |
| rAAV | 1009 | WO2016081811A1 SEQ ID NO: 15 |
| rAAV | 1010 | WO2016081811A1 SEQ ID NO: 16 |
| rAAV | 1011 | WO2016081811A1 SEQ ID NO: 17 |
| rAAV | 1012 | WO2016081811A1 SEQ ID NO: 18 |
| rAAV | 1013 | WO2016081811A1 SEQ ID NO: 19 |
| rAAV | 1014 | WO2016081811A1 SEQ ID NO: 20 |
| rAAV | 1015 | WO2016081811A1 SEQ ID NO: 21 |
| rAAV | 1016 | WO2016081811A1 SEQ ID NO: 22 |
| rAAV | 1017 | WO2016081811A1 SEQ ID NO: 23 |
| rAAV | 1018 | WO2016081811A1 SEQ ID NO: 24 |
| rAAV | 1019 | WO2016081811A1 SEQ ID NO: 25 |
| rAAV | 1020 | WO2016081811A1 SEQ ID NO: 26 |
| rAAV | 1021 | WO2016081811A1 SEQ ID NO: 27 |
| rAAV | 1022 | WO2016081811A1 SEQ ID NO: 28 |
| rAAV | 1023 | WO2016081811A1 SEQ ID NO: 29 |
| rAAV | 1024 | WO2016081811A1 SEQ ID NO: 30 |
| rAAV | 1025 | WO2016081811A1 SEQ ID NO: 31 |
| rAAV | 1026 | WO2016081811A1 SEQ ID NO: 32 |
| rAAV | 1027 | WO2016081811A1 SEQ ID NO: 33 |
| rAAV | 1028 | WO2016081811A1 SEQ ID NO: 34 |
| rAAV | 1029 | WO2016081811A1 SEQ ID NO: 35 |
| rAAV | 1030 | WO2016081811A1 SEQ ID NO: 36 |
| rAAV | 1031 | WO2016081811A1 SEQ ID NO: 37 |
| rAAV | 1032 | WO2016081811A1 SEQ ID NO: 38 |
| rAAV | 1033 | WO2016081811A1 SEQ ID NO: 39 |
| rAAV | 1034 | WO2016081811A1 SEQ ID NO: 40 |
| rAAV | 1035 | WO2016081811A1 SEQ ID NO: 41 |
| rAAV | 1036 | WO2016081811A1 SEQ ID NO: 42 |
| rAAV | 1037 | WO2016081811A1 SEQ ID NO: 43 |
| rAAV | 1038 | WO2016081811A1 SEQ ID NO: 44 |
| rAAV | 1039 | WO2016081811A1 SEQ ID NO: 45 |
| rAAV | 1040 | WO2016081811A1 SEQ ID NO: 46 |
| rAAV | 1041 | WO2016081811A1 SEQ ID NO: 47 |
| rAAV | 1042 | WO2016081811A1 SEQ ID NO: 48 |
| rAAV | 1043 | WO2016081811A1 SEQ ID NO: 49 |
| rAAV | 1044 | WO2016081811A1 SEQ ID NO: 50 |
| rAAV | 1045 | WO2016081811A1 SEQ ID NO: 51 |
| rAAV | 1046 | WO2016081811A1 SEQ ID NO: 52 |
| rAAV | 1047 | WO2016081811A1 SEQ ID NO: 53 |
| rAAV | 1048 | WO2016081811A1 SEQ ID NO: 54 |
| rAAV | 1049 | WO2016081811A1 SEQ ID NO: 55 |
| rAAV | 1050 | WO2016081811A1 SEQ ID NO: 56 |
| rAAV | 1051 | WO2016081811A1 SEQ ID NO: 57 |
| rAAV | 1052 | WO2016081811A1 SEQ ID NO: 58 |
| rAAV | 1053 | WO2016081811A1 SEQ ID NO: 59 |
| rAAV | 1054 | WO2016081811A1 SEQ 1D NO: 60 |
| rAAV | 1055 | WO2016081811A1 SEQ ID NO: 61 |
| rAAV | 1056 | WO2016081811A1 SEQ ID NO: 62 |
| rAAV | 1057 | WO2016081811A1 SEQ ID NO: 63 |
| rAAV | 1058 | WO2016081811A1 SEQ ID NO: 64 |
| rAAV | 1059 | WO2016081811A1 SEQ ID NO: 65 |
| rAAV | 1060 | WO2016081811A1 SEQ ID NO: 66 |
| rAAV | 1061 | WO2016081811A1 SEQ ID NO: 67 |
| rAAV | 1062 | WO2016081811A1 SEQ ID NO: 68 |
| rAAV | 1063 | WO2016081811A1 SEQ ID NO: 69 |
| rAAV | 1064 | WO2016081811A1 SEQ ID NO: 70 |
| rAAV | 1065 | WO2016081811A1 SEQ ID NO: 71 |
| rAAV | 1066 | WO2016081811A1 SEQ ID NO: 72 |
| rAAV | 1067 | WO2016081811A1 SEQ ID NO: 73 |
| rAAV | 1068 | WO2016081811A1 SEQ ID NO: 74 |
| rAAV | 1069 | WO2016081811A1 SEQ ID NO: 75 |
| rAAV | 1070 | WO2016081811A1 SEQ ID NO: 76 |
| rAAV | 1071 | WO2016081811A1 SEQ ID NO: 77 |
| rAAV | 1072 | WO2016081811A1 SEQ ID NO: 78 |
| rAAV | 1073 | WO2016081811A1 SEQ ID NO: 79 |
| rAAV | 1074 | WO2016081811A1 SEQ ID NO: 80 |
| rAAV | 1075 | WO2016081811A1 SEQ ID NO: 81 |
| rAAV | 1076 | WO2016081811A1 SEQ ID NO: 82 |
| rAAV | 1077 | WO2016081811A1 SEQ ID NO: 83 |
| rAAV | 1078 | WO2016081811A1 SEQ ID NO: 84 |
| rAAV | 1079 | WO2016081811A1 SEQ ID NO: 85 |
| rAAV | 1080 | WO2016081811A1 SEQ ID NO: 86 |
| rAAV | 1081 | WO2016081811A1 SEQ ID NO: 87 |
| rAAV | 1082 | WO2016081811A1 SEQ ID NO: 88 |
| rAAV | 1083 | WO2016081811A1 SEQ ID NO: 89 |
| rAAV | 1084 | WO2016081811A1 SEQ ID NO: 90 |
| rAAV | 1085 | WO2016081811A1 SEQ ID NO: 91 |
| rAAV | 1086 | WO2016081811A1 SEQ ID NO: 92 |
| rAAV | 1087 | WO2016081811A1 SEQ ID NO: 93 |
| rAAV | 1088 | WO2016081811A1 SEQ ID NO: 94 |
| rAAV | 1089 | WO2016081811A1 SEQ ID NO: 95 |
| rAAV | 1090 | WO2016081811A1 SEQ ID NO: 96 |
| rAAV | 1091 | WO2016081811A1 SEQ ID NO: 97 |
| rAAV | 1092 | WO2016081811A1 SEQ ID NO: 98 |
| rAAV | 1093 | WO2016081811A1 SEQ ID NO: 99 |
| rAAV | 1094 | WO2016081811A1 SEQ ID NO: 100 |
| rAAV | 1095 | WO2016081811A1 SEQ ID NO: 101 |
| rAAV | 1096 | WO2016081811A1 SEQ ID NO: 102 |
| rAAV | 1097 | WO2016081811A1 SEQ ID NO: 103 |
| rAAV | 1098 | WO2016081811A1 SEQ ID NO: 104 |
| rAAV | 1099 | WO2016081811A1 SEQ ID NO: 105 |
| rAAV | 1100 | WO2016081811A1 SEQ ID NO: 106 |
| rAAV | 1101 | WO2016081811A1 SEQ ID NO: 107 |
| rAAV | 1102 | WO2016081811A1 SEQ ID NO: 108 |
| rAAV | 1103 | WO2016081811A1 SEQ ID NO: 109 |
| rAAV | 1104 | WO2016081811A1 SEQ ID NO: 110 |
| rAAV | 1105 | WO2016081811A1 SEQ ID NO: 111 |
| rAAV | 1106 | WO2016081811A1 SEQ ID NO: 112 |
| rAAV | 1107 | WO2016081811A1 SEQ ID NO: 113 |
| rAAV | 1108 | WO2016081811A1 SEQ ID NO: 114 |
| rAAV | 1109 | WO2016081811A1 SEQ ID NO: 115 |
| rAAV | 1110 | WO2016081811A1 SEQ ID NO: 116 |
| rAAV | 1111 | WO2016081811A1 SEQ ID NO: 117 |
| rAAV | 1112 | WO2016081811A1 SEQ ID NO: 118 |
| rAAV | 1113 | WO2016081811A1 SEQ ID NO: 119 |
| rAAV | 1114 | WO2016081811A1 SEQ ID NO: 120 |
| rAAV | 1115 | WO2016081811A1 SEQ ID NO: 121 |
| rAAV | 1116 | WO2016081811A1 SEQ ID NO: 122 |
| rAAV | 1117 | WO2016081811A1 SEQ ID NO: 123 |
| rAAV | 1118 | WO2016081811A1 SEQ ID NO: 124 |
| rAAV | 1119 | WO2016081811A1 SEQ ID NO: 125 |
| rAAV | 1120 | WO2016081811A1 SEQ ID NO: 126 |
| rAAV | 1121 | WO2016081811A1 SEQ ID NO: 127 |
| rAAV | 1122 | WO2016081811A1 SEQ ID NO: 128 |
| AAV8 E532K | 1123 | WO2016081811A1 SEQ ID NO: 133 |
| AAV8 E532K | 1124 | WO2016081811A1 SEQ ID NO: 134 |
| rAAV4 | 1125 | WO2016115382A1 SEQ ID NO: 2 |
| rAAV4 | 1126 | WO2016115382A1 SEQ ID NO: 3 |
| rAAV4 | 1127 | WO2016115382A1 SEQ ID NO: 4 |
| rAAV4 | 1128 | WO2016115382A1 SEQ ID NO: 5 |
| rAAV4 | 1129 | WO2016115382A1 SEQ ID NO: 6 |
| rAAV4 | 1130 | WO2016115382A1 SEQ ID NO: 7 |
| rAAV4 | 1131 | WO2016115382A1 SEQ ID NO: 8 |
| rAAV4 | 1132 | WO2016115382A1 SEQ ID NO: 9 |
| rAAV4 | 1133 | WO2016115382A1 SEQ ID NO: 10 |
| rAAV4 | 1134 | WO2016115382A1 SEQ ID NO: 11 |
| rAAV4 | 1135 | WO2016115382A1 SEQ ID NO: 12 |
| rAAV4 | 1136 | WO2016115382A1 SEQ ID NO: 13 |
| rAAV4 | 1137 | WO2016115382A1 SEQ ID NO: 14 |
| rAAV4 | 1138 | WO2016115382A1 SEQ ID NO: 15 |
| rAAV4 | 1139 | WO2016115382A1 SEQ ID NO: 16 |
| rAAV4 | 1140 | WO2016115382A1 SEQ ID NO: 17 |
| rAAV4 | 1141 | WO2016115382A1 SEQ ID NO: 18 |
| rAAV4 | 1142 | WO2016115382A1 SEQ ID NO: 19 |
| rAAV4 | 1143 | WO2016115382A1 SEQ ID NO: 20 |
| rAAV4 | 1144 | WO2016115382A1 SEQ ID NO: 21 |
| AAV11 | 1145 | WO2016115382A1 SEQ ID NO: 22 |
| AAV12 | 1146 | WO2016115382A1 SEQ ID NO: 23 |
| rh32 | 1147 | WO2016115382A1 SEQ ID NO: 25 |
| rh33 | 1148 | WO2016115382A1 SEQ ID NO: 26 |
| rh34 | 1149 | WO2016115382A1 SEQ ID NO: 27 |
| rAAV4 | 1150 | WO2016115382A1 SEQ ID NO: 28 |
| rAAV4 | 1151 | WO2016115382A1 SEQ ID NO: 29 |
| rAAV4 | 1152 | WO2016115382A1 SEQ ID NO: 30 |
| rAAV4 | 1153 | WO2016115382A1 SEQ ID NO: 31 |
| rAAV4 | 1154 | WO2016115382A1 SEQ ID NO: 32 |
| rAAV4 | 1155 | WO2016115382A1 SEQ ID NO: 33 |
| AAV2/8 | 1156 | WO2016131981A1 SEQ ID NO: 47 |
| AAV2/8 | 1157 | WO2016131981A1 SEQ ID NO: 48 |
| ancestral AAV | 1158 | WO2016154344A1 SEQ ID NO: 7 |
| ancestral AAV variant C4 | 1159 | WO2016154344A1 SEQ ID NO: 13 |
| ancestral AAV variant C7 | 1160 | WO2016154344A1 SEQ ID NO: 14 |
| ancestral AAV variant G4 | 1161 | WO2016154344A1 SEQ ID NO: 15 |
| consensus amino acid | 1162 | WO2016154344A1 SEQ ID NO: 16 |
| sequence of ancestral AAV | | |
| variants, C4, C7 and G4 | | |
| consensus amino acid | 1163 | WO2016154344A1 SEQ ID NO: 17 |
| sequence of ancestral AAV | | |
| variants, C4 and C7 | | |
| AAV8 (with a AAV2 | 1164 | WO2016150403A1 SEQ ID NO: 13 |
| phospholipase domain) | | |
| AAV VR-942n | 1165 | US20160289275A1 SEQ ID NO: 10 |
| AAV5-A (M569V) | 1166 | US20160289275A1 SEQ ID NO: 13 |
| AAV5-A (M569V) | 1167 | US20160289275A1 SEQ ID NO: 14 |
| AAV5-A (Y585V) | 1168 | US20160289275A1 SEQ ID NO: 16 |
| AAV5-A (Y585V) | 1169 | US20160289275A1 SEQ ID NO: 17 |
| AAV5-A (L587T) | 1170 | US20160289275A1 SEQ ID NO: 19 |
| AAV5-A (L587T) | 1171 | US20160289275A1 SEQ ID NO: 20 |
| AAV5-A (Y585V/L587T) | 1172 | US20160289275A1 SEQ ID NO: 22 |
| AAV5-A (Y585V/L587T) | 1173 | US20160289275A1 SEQ ID NO: 23 |
| AAV5-B (D652A) | 1174 | US20160289275A1 SEQ ID NO: 25 |
| AAV5-B (D652A) | 1175 | US20160289275A1 SEQ ID NO: 26 |
| AAV5-B (T362M) | 1176 | US20160289275A1 SEQ ID NO: 28 |
| AAV5-B (T362M) | 1177 | US20160289275A1 SEQ ID NO: 29 |
| AAV5-B (Q359D) | 1178 | US20160289275A1 SEQ ID NO: 31 |
| AAV5-B (Q359D) | 1179 | US20160289275A1 SEQ ID NO: 32 |
| AAV5-B (E350Q) | 1180 | US20160289275A1 SEQ ID NO: 34 |
| AAV5-B (E350Q) | 1181 | US20160289275A1 SEQ ID NO: 35 |
| AAV5-B (P533S) | 1182 | US20160289275A1 SEQ ID NO: 37 |
| AAV5-B (P533S) | 1183 | US20160289275A1 SEQ ID NO: 38 |
| AAV5-B (P533G) | 1184 | US20160289275A1 SEQ ID NO: 40 |
| AAV5-B (P533G) | 1185 | US20160289275A1 SEQ ID NO: 41 |
| AAV5-mutation in loop VII | 1186 | US20160289275A1 SEQ ID NO: 43 |
| AAV5-mutation in loop VII | 1187 | US20160289275A1 SEQ ID NO: 44 |
| AAV8 | 1188 | US20160289275A1 SEQ ID NO: 47 |
| Mut A (LK03/AAV8) | 1189 | WO2016181123A1 SEQ lD NO: 1 |
| Mut B (LK03/AAV5) | 1190 | WO2016181123A1 SEQ ID NO: 2 |
| Mut C (AAV8/AAV3B) | 1191 | WO2016181123A1 SEQ ID NO: 3 |
| Mut D (AAV5/AAV3B) | 1192 | WO2016181123A1 SEQ ID NO: 4 |
| Mut E (AAV8/AAV3B) | 1193 | WO2016181123A1 SEQ ID NO: 5 |
| Mut F (AAV3B/AAV8) | 1194 | WO2016181123A1 SEQ lD NO: 6 |
| AAV44.9 | 1195 | WO2016183297A1 SEQ ID NO: 4 |
| AAV44.9 | 1196 | WO2016183297A1 SEQ ID NO: 5 |
| AAVrh8 | 1197 | WO2016183297A1 SEQ ID NO: 6 |
| AAV44.9 (S470N) | 1198 | WO2016183297A1 SEQ ID NO: 9 |
| rh74 VP1 | 1199 | US20160375110A1 SEQ ID NO: 1 |
| AAV-LK03 (L125I) | 1200 | WO2017015102A1 SEQ ID NO: 5 |
| AAV3B (S663V + T492V) | 1201 | WO2017015102A1 SEQ ID NO: 6 |
| Anc80 | 1202 | WO2017019994A2 SEQ ID NO: 1 |
| Anc80 | 1203 | WO2017019994A2 SEQ ID NO: 2 |
| Anc81 | 1204 | WO2017019994A2 SEQ ID NO: 3 |
| Anc81 | 1205 | WO2017019994A2 SEQ ID NO: 4 |
| Anc82 | 1206 | WO2017019994A2 SEQ ID NO: 5 |
| Anc82 | 1207 | WO2017019994A2 SEQ ID NO: 6 |
| Anc83 | 1208 | WO2017019994A2 SEQ ID NO: 7 |
| Anc83 | 1209 | WO2017019994A2 SEQ ID NO: 8 |
| Anc84 | 1210 | WO2017019994A2 SEQ ID NO: 9 |
| Anc84 | 1211 | WO2017019994A2 SEQ ID NO: 10 |
| Anc94 | 1212 | WO2017019994A2 SEQ ID NO: 11 |
| Anc94 | 1213 | WO2017019994A2 SEQ ID NO: 12 |
| Anc113 | 1214 | WO2017019994A2 SEQ ID NO: 13 |
| Anc113 | 1215 | WO2017019994A2 SEQ ID NO: 14 |
| Anc126 | 1216 | WO2017019994A2 SEQ ID NO: 15 |
| Anc126 | 1217 | WO2017019994A2 SEQ ID NO: 16 |
| Anc127 | 1218 | WO2017019994A2 SEQ ID NO: 17 |
| Anc127 | 1219 | WO2017019994A2 SEQ ID NO: 18 |
| Anc80L27 | 1220 | WO2017019994A2 SEQ ID NO: 19 |
| Anc80L59 | 1221 | WO2017019994A2 SEQ ID NO: 20 |
| Anc80L60 | 1222 | WO2017019994A2 SEQ ID NO: 21 |
| Anc80L62 | 1223 | WO2017019994A2 SEQ ID NO: 22 |
| Anc80L65 | 1224 | WO2017019994A2 SEQ ID NO: 23 |
| Anc80L33 | 1225 | WO2017019994A2 SEQ ID NO: 24 |
| Anc80L36 | 1226 | WO2017019994A2 SEQ ID NO: 25 |
| Anc80L44 | 1227 | WO2017019994A2 SEQ ID NO: 26 |
| Anc80L1 | 1228 | WO2017019994A2 SEQ ID NO: 35 |
| Anc80L1 | 1229 | WO2017019994A2 SEQ ID NO: 36 |
| AAVrh10 | 1230 | WO2017019994A2 SEQ ID NO: 41 |
| Anc110 | 1231 | WO2017019994A2 SEQ ID NO: 42 |
| Anc110 | 1232 | WO2017019994A2 SEQ ID NO: 43 |
| AAVrh32.33 | 1233 | WO2017019994A2 SEQ ID NO: 45 |
| AAVrh74 | 1234 | WO2017049031A1 SEQ ID NO: 1 |
| AAV2 | 1235 | WO2017053629A2 SEQ ID NO: 49 |
| AAV2 | 1236 | WO2017053629A2 SEQ ID NO: 50 |
| AAV2 | 1237 | WO2017053629A2 SEQ ID NO: 82 |
| Parvo-like virus | 1238 | WO2017070476A2 SEQ ID NO: 1 |
| Parvo-like virus | 1239 | WO2017070476A2 SEQ ID NO: 2 |
| Parvo-like virus | 1240 | WO2017070476A2 SEQ ID NO: 3 |
| Parvo-like virus | 1241 | WO2017070476A2 SEQ ID NO: 4 |
| Parvo-like virus | 1242 | WO2017070476A2 SEQ ID NO: 5 |
| Parvo-like virus | 1243 | WO2017070476A2 SEQ ID NO: 6 |
| AAVrh.10 | 1244 | WO2017070516A1 SEQ ID NO: 7 |
| AAVrh.10 | 1245 | WO2017070516A1 SEQ ID NO: 14 |
| AAV2tYF | 1246 | WO2017070491A1 SEQ ID NO: 1 |
| AAV-SPK | 1247 | WO2017075619A1 SEQ ID NO:28 |
| AAV2.5 | 1248 | US20170128528A1 SEQ ID NO: 13 |
| AAV1.1 | 1249 | US20170128528A1 SEQ ID NO: 15 |
| AAV6.1 | 1250 | US20170128528A1 SEQ ID NO: 17 |
| AAV6.3.1 | 1251 | US20170128528A1 SEQ ID NO: 18 |
| AAV2i8 | 1252 | US20170128528A1 SEQ ID NO: 28 |
| AAV2i8 | 1253 | US20170128528A1 SEQ ID NO: 29 |
| ttAAV | 1254 | US20170128528A1 SEQ ID NO: 30 |
| ttAAV-S312N | 1255 | US20170128528A1 SEQ ID NO: 32 |
| ttAAV-S312N | 1256 | US20170128528A1 SEQ ID NO: 33 |
| AAV6 (Y705, Y731, and | 1257 | WO2016134337A1 SEQ ID NO: 24 |
| T492) | | |
| AAV2 | 1258 | WO2016134375A1 SEQ ID NO: 9 |
| AAV2 | 1259 | WO2016134375A1 SEQ ID NO: 10 |

The contents of each of the patents, applications, and/or publications listed in Table 1 are incorporated herein by reference in their entireties as related to AAV capsids, insofar as they do not conflict with the present disclosure.

In certain embodiments, the AAV serotype may be, or may comprise a sequence as described in International Patent Publication WO2015038958 (the content of which is incorporated herein by reference in its entirety as related to AAV capsids, insofar as it does not conflict with the present disclosure), such as, but not limited to, AAV9 (SEQ ID NO: 2 and 11 of WO2015038958 or SEQ ID NO: 135 and 136 herein), PHP.B (SEQ ID NO: 8 and 9 of WO2015038958, herein SEQ ID NO: 3 and 4), G2B-13 (SEQ ID NO: 12 of WO2015038958, herein SEQ ID NO: 5), G2B-26 (SEQ ID NO: 13 of WO2015038958, herein SEQ ID NO: 3), TH1.1-32 (SEQ ID NO: 14 of WO2015038958, herein SEQ ID NO: 6), TH1.1-35 (SEQ ID NO: 15 of WO2015038958, herein SEQ ID NO: 7), or variants thereof. Further, any of the “targeting peptides” or “amino acid inserts” (used herein interchangeably to mean sequences that may be inserted into an AAV capsid sequence to facilitate delivery to CNS tissue) described in WO2015038958, may be inserted into any parent AAV serotype, such as, but not limited to, AAV9 (SEQ ID NO: 135 for the DNA sequence and SEQ ID NO: 136 for the amino acid sequence). In certain embodiments, the amino acid insert is inserted between amino acids 586-592 of the parent AAV (e.g., AAV9), In certain embodiments, the amino acid insert is inserted between amino acids 588-589 of the parent AAV sequence. The amino acid insert may be, but is not limited to, any of the following amino acid sequences. TLAVPFK (SEQ ID NO: 1 of WO2015038958; herein SEQ ID NO: 1260), KFPVALT (SEQ ID NO: 3 of WO2015038958; herein SEQ ID NO: 1261), LAVPFK (SEQ ID NO: 31 of WO2015038958; herein SEQ ID NO: 1262), AVPFK (SEQ ID NO: 32 of WO2015038958; herein SEQ ID NO: 1263), VPFK (SEQ ID NO: 33 of WO2015038958; herein SEQ ID NO: 1264), TLAVPF (SEQ ID NO: 34 of WO2015038958; herein SEQ ID NO: 1265), TLAVP (SEQ ID NO: 35 of WO2015038958; herein SEQ ID NO: 1266), TLAV (SEQ ID NO: 36 of WO2015038958; herein SEQ ID NO: 1267), SVSKPFL (SEQ ID NO: 28 of WO2015038958; herein SEQ ID NO: 1268), FTLTTPK (SEQ ID NO: 29 of WO2015038958; herein SEQ ID NO: 1269), MNATKNV (SEQ ID NO: 30 of WO2015038958; herein SEQ ID NO: 1270), QSSQTPR (SEQ ID NO: 54 of WO2015038958; herein SEQ ID NO: 1271), ILGTGTS (SEQ ID NO: 55 of WO2015038958; herein SEQ ID NO: 1272), TRTNPEA (SEQ ID NO: 56 of WO2015038958; herein SEQ ID NO: 1273), NGGTSSS (SEQ ID NO: 58 of WO2015038958; herein SEQ ID NO: 1274), or YTLSQGW (SEQ ID NO: 60 of WO2015038958; herein SEQ ID NO: 1275). Non-limiting examples of nucleotide sequences that may encode the amino acid inserts comprise, but are not limited to, the following, AAGTTTCCTGTGGCGTTGACT (for SEQ ID NO: 3 of WO2015038958; herein SEQ ID NO: 1276), ACTTTGGCGGTGCCTTTTAAG (SEQ ID NO: 24 and 49 of WO2015038958; herein SEQ ID NO: 1277), AGTGTGAGTAAGCCTTTTTTG (SEQ ID NO: 25 of WO2015038958; herein SEQ ID NO: 1278), TTTACGTTGACGACGCCTAAG (SEQ ID NO: 26 of WO2015038958; herein SEQ ID NO: 1279), ATGAATGCTACGAAGAATGTG (SEQ ID NO: 27 of WO2015038958; herein SEQ ID NO: 1280), CAGTCGTCGCAGACGCCTAGG (SEQ ID NO: 48 of WO2015038958; herein SEQ ID NO: 1281)-.NTTCIGGGGACIGGIACTTCG (SEQ ID NO: 50 and 52 of WO2015038958; herein SEQ ID NO: 1282), ACGCGGACTAATCCTGAGGCT (SEQ ID NO: 51 of WO2015038958; herein SEQ ID NO: 1283), AATGGGGGGACTAGTAGTTCT (SEQ ID NO: 53 of WO2015038958; herein SEQ IL) NO: 1284), or TATACTTTGTCGCAGGGTTGG (SEQ ID NO: 59 of WO2015038958; herein SEQ ID NO: 1285).

In certain embodiments, the AAV serotype may be, or may comprise a sequence as described in International Patent Publication WO2017100671 (the content of which is incorporated herein by reference in its entirety as related to AAV capsids, insofar as it does not conflict with the present disclosure), such as, but not limited to, AAV9 K449R (SEQ ID NO: 45 of WO2017100671, herein SEQ ID NO: 9), PHP.N (SEQ ID NO: 46 of WO2017100671, herein SEQ NO: 2), PHP.S (SEQ ID NO: 47 of WO2017100671, herein SEQ ID NO: 8), or variants thereof. Further, any of the targeting peptides or amino acid inserts described in WO2017100671 may be inserted into any parent AAV serotype, such as, but not limited to, AAV9 (SEQ ID NO: 9 or SEQ ID NO: 136). In certain embodiments, the amino acid insert is inserted between amino acids 586-592 of the parent AAV (e.g., AAV9). In certain embodiments, the amino acid insert is inserted between amino acids 588-589 of the parent AAV sequence. The amino acid insert may be, but is not limited to, any of the following amino acid sequences, AQTLAVPFKAQ (SEQ ID NO: 1 of WO2017100671; herein SEQ ID NO: 1286), AQSVSKPFLAQ (SEQ ID NO: 2 of WO2017100671; herein SEQ ID NO: 1287)_{;} AQFTLTTPKAQ (SEQ ID NO: 3 in the sequence listing of WO2017100671; herein SEQ ID NO: 1288), DGTLAVPFKAQ (SEQ ID NO: 4 in the sequence listing of WO2017100671; herein SEQ ID NO: 1289), ESTIAVPFKAQ (SEQ ID NO: 5 of WO2017100671; herein SEQ ID NO: 1290), GGTLAVPFKAQ (SEQ ID NO: 6 of WO2017100671; herein SEQ ID NO: 1291), AQTLATPFKAQ (SEQ ID NO: 7 and 33 of WO2017100671; herein SEQ ID NO: 1292), ATTLATPFICAQ (SEQ ID NO: 8 of WO2017100671; herein SEQ ID NO: 1293), DGILATPFKAQ (SEQ ID NO: 9 of WO2017100671; herein SEQ ID NO: 1294), GGTLATPFKAQ (SEQ ID NO: 10 of WO2017100671; herein SEQ ID NO: 1295), SGSLAVPFKAQ (SEQ ID NO: 11 of WO2017100671; herein SEQ ID NO: 1296), AQTLAQPFKAQ (SEQ ID NO: 12 of WO2017100671; herein SEQ ID NO: 1297), AQTLQQPFKAQ (SEQ ID NO: 13 of WO2017100671; herein SEQ ID NO: 1298), AQTLSNPFKAQ (SEQ ID NO: 14 of WO2017100671; herein SEQ ID NO: 1299), AQTLAVPFSNP (SEQ ID NO: 15 of WO2017100671; herein SEQ ID NO: 1300), QGILAVPFKAQ (SEQ ID NO: 16 of WO2017100671; herein SEQ ID NO: 1301), NQTLAVPFKAQ (SEQ ID NO: 17 of WO2017100671; herein SEQ ID NO: 1302), EGSLAVPFKAQ (SEQ ID NO: 18 of WO2017100671; herein SEQ ID NO: 1303), SGNIAVPFKAQ (SEQ ID NO: 19 of WO2017100671; herein SEQ ID NO: 1304), EGTLAVPFKAQ (SEQ ID NO: 20 of WO2017100671; herein SEQ ID NO: 1305), DSTLAVPFKAQ (SEQ ID NO: 21 in Table 1 of WO2017100671; herein SEQ ID NO: 1306), AVTLAVPFKAQ (SEQ ID NO: 22 of WO2017100671; herein SEQ ID NO: 1307), AQTLPQPFKAQ (SEQ ID NO: 23 of WO2017100671; herein SEQ ID NO: 1308), AQTLPQPFKAQ (SEQ ID NO: 24 and 32 of WO2017100671; herein SEQ ID NO: 1309), AQTLSQPFKAQ (SEQ ID NO: 25 of WO2017100671; herein SEQ ID NO: 1310), AQTLQLPFKAQ (SEQ ID NO: 26 of WO2017100671; herein SEQ ID NO: 1311), AQTLIMPFKAQ (SEQ IL) NO: 27, and 34 of WO2017100671 and SEQ ID NO: 35 in the sequence listing of WO2017100671; herein SEQ ID NO: 1312), AQTLTTPFKAQ (SEQ ID NO: 28 of WO2017100671; herein SEQ ID NO: 1313), AQYTLSQGWAQ (SEQ ID NO: 29 of WO2017100671; herein SEQ ID NO: 1314), AQMNATKNVAQ (SEQ ID NO: 30 of WO2017100671; herein SEQ ID NO: 1315), AQVSGGHESAQ (SEQ ID NO: 31 of WO2017100671; herein SEQ ID NO: 1316), AQTLTTPFKAQ (SEQ ID NO: 35 in Table 1 of WO2017100671; herein SEQ ID NO: 1317), AQTLSKPFKAQ (SEQ ID NO: 36 of WO2017100671; herein SEQ ID NO: 1318), QAVRTSL (SEQ ID NO: 37 of WO2017100671; herein SEQ ID NO: 1319), YTLSQGW (SEQ ID NO: 38 of WO201.7100671.; herein SEQ ID NO: 1275), LAKERLS (SEQ ID NO: 39 of WO2017100671; herein SEQ ID NO: 1320), TLAVPFK (SEQ ID NO: 40 in the sequence listing of WO2017100671; herein SEQ ID NO: 1260), SVSKPFL (SEQ ID NO: 41 of WO2017100671; herein SEQ ID NO: 1268), FTLTTPK (SEQ ID NO: 42 of WO2017100671; herein SEQ ID NO: 1269), MNSTKNV (SEQ ID NO: 43 of WO2017100671; herein SEQ ID NO: 1321), VSGGHHS (SEQ ID NO: 44 of WO2017100671; herein SEQ ID NO: 1322), SAQTILAVPFKAQAQ (SEQ ID NO: 48 of WO2017100671; herein SEQ ID NO: 1323), SXXXLAVPFKAQAQ (SEQ ID NO: 49 of WO2017100671 wherein X may be any amino acid; herein SEQ ID NO: 1324), SAQXXXVPFKAQAQ (SEQ IL) NO: 50 of WO2017100671 wherein X may be any amino acid; herein SEQ ID NO: 1325), SAQTLXXXFKAQAQ (SEQ ID NO: 51 of WO2017100671 wherein X may be any amino acid; herein SEQ ID NO: 1326), SAQTLAVXXXAQAQ (SEQ ID NO: 52 of WO2017100671 wherein X may be any amino acid; herein SEQ ID NO: 1327), SAQTLAVPFXXXAQ (SEQ ID NO: 53 of WO2017100671 wherein X may be any amino acid; herein SEQ ID NO: 1328), TNHQSAQ (SEQ ID NO: 65 of WO2017100671; herein SEQ ID NO: 1329), AQAQTGW (SEQ ID NO: 66 of WO2017100671; herein SEQ ID NO: 1330), DGTLATPFK (SEQ ID NO: 67 of WO2017100671; herein SEQ ID NO: 1331), DGTLATPFKXX (SEQ ID NO: 68 of WO2017100671 wherein X may be any amino acid; herein SEQ ID NO: 1332), LAVPFKAQ (SEQ ID NO: 80 of WO2017100671; herein SEQ ID NO: 1333), VPFKAQ (SEQ ID NO: 81 of WO201.7100671; herein SEQ ID NO: 1334), FKAQ (SEQ ID NO: 82 of WO2017100671; herein SEQ ID NO: 1335), AQTLAV (SEQ ID NO: 83 of WO2017100671; herein SEQ ID NO: 1336), AQTLAVPF (SEQ m NO: 84 of WO2017100671; herein SEQ ID NO: 1337), QAVR (SEQ ID NO: 85 of WO2017100671; herein SEQ ID NO: 1338), AVRT (SEQ ID NO: 86 of WO2017100671; herein SEQ ID NO: 1339), VRTS (SEQ ID NO: 87 of WO2017100671; herein SEQ ID NO: 1340), RTSI, (SEQ ID NO: 88 of WO2017100671; herein SEQ ID NO: 1341), QAVRT (SEQ ID NO: 89 of WO2017100671; herein SEQ ID NO: 1342), AVRTS (SEQ ID NO: 90 of WO2017100671; herein SEQ ID NO: 1343), VRTSL (SEQ ID NO: 91 of WO2017100671; herein SEQ ID NO: 1344), QAVRTS (SEQ NO: 92 of WO2017100671; herein SEQ ID NO: 1345), or AVRTSI, (SEQ ID NO: 93 of WO2017100671; herein SEQ ID NO: 1346).

Non-limiting examples of nucleotide sequences that may encode the amino acid inserts comprise the following, GATGGGACTTTGGCGGTGCCTTTTAAGGCACAG (SEQ ID NO: 54 of WO2017100671; herein SEQ ID NO: 1347), GATGGGACGTTGGCGGTGCCITITAAGGCACAG (SEQ ID NO: 55 of WO2017100671; herein SEQ ID NO: 1348), CAGGCGGITAGGACGICTIT (SEQ NO: 56 of WO2017100671; herein SEQ ID NO: 1349), CAGGTCTTCACGGACTCAGACTATCAG (SEQ ID NO: 57 and 78 of WO2017100671; herein SEQ ID NO: 1350), CAAGTAAAACCTCTACAAAIGTGGTAAAATCG (SEQ ID NO: 58 of WO2017100671; herein SEQ ID NO: 1351); ACTCATCGACCAATACTTGTACTATCTCTCTAGAAC (SEQ ID NO: 59 of WO2017100671; herein SEQ ID NO: 1352), GGAAGTATICCTIGGTTTTGAACCCA (SEQ ID NO: 60 of 402017100671; herein SEQ ID NO: 1353), GGTCGCGGTTCTTGTTTGTGGAT (SEQ ID NO: 61 of WO2017100671; herein SEQ ID NO: 1354). CGACCTTGAAGCGCATGAACTCCT (SEQ ID NO: 62 of WO2017100671; herein SEQ ID NO: 1355), GTATTCCTTGGTTTTGAACCCAACCGGTCTGCGCCTGTGCMNNMNNMNNMNNM NNMNNMNNTTGGGCACTCTGGTGGTTTGTG (SEQ ID NO: 63 of WO2017100671 wherein N may be A, C, T, or G; herein SEQ ID NO: 1356), GTATTCCTTGGTFITGAACCCAACCGGTCTGCGCMNNMNNMNNAAAAGGCACCG CCAAAGTTTG (SEQ ID NO: 69 of WO2017100671 wherein N may he A, C, T, or G; herein SEQ ID NO: 1357), GTATTCCTTGGTTTTGAACCCAACCGGTCTGCGCCTGTGCMNNMNNMNNCACCG CCAAAGTTTGGGCACT (SEQ ID NO: 70 of WO2017100671 wherein N may be A, C, T, or G; herein SEQ ID NO: 1358), GTACCTTGGTTTTGAACCCAACCGGICTGCGCCTGTGCCTTAAAMNNNINNMN NCAAAGTTTGGGCACTCTGGTGG (SEQ ID NO: 71 of WO2017100671 wherein N may be A, C, T, or G; herein SEQ ID NO: 1359), GTATTCCTTGGTTTI GAACCCAACCGGTCTGCGCCTUTGCCTTAAAAGGCACMNN MNNMNNTTGGGCACTCTGGTGGTITGTG (SEQ ID NO: 72 of WO2017100671 wherein N may be A, C, T, or G; herein SEQ ID NO: 1360), ACTTIGGCGGTGCCTTTTAAG (SEQ ID NO: 74 of WO2017100671; herein SEQ ID NO: 1277), AGTGTGAGTAAGCCTTTTTTG (SEQ ID NO: 75 of WO2017100671; herein SEQ ID NO: 1278), TTTACGTTGACGACGCCTAAG (SEQ ID NO: 76 of WO2017100671; herein SEQ ID NO: 1279), TATACTTTGTCGCAGGGTTGG (SEQ ID NO: 77 of WO2017100671; herein SEQ ID NO: 1285), or CTTGCGAAGGAGCGGCTI TCG (SEQ ID NO: 79 of WO2017100671; herein SEQ ID NO: 1361).

In certain embodiments, the AAV serotype may be, or may comprise a sequence as described in U.S. Pat. No. 9,624,274 (the content of which is incorporated herein by reference in its entirety as related to AAV capsids, insofar as it does not conflict with the present disclosure), such as, but not limited to, AAV1 (SEQ ID NO: 181 of U.S. Pat. No. 9,624,274), AAV6 (SEQ ID NO: 182 of U.S. Pat. No. 9,624,274), AAV2 (SEQ ID NO: 183 of U.S. Pat. No. 9,624,274), AAV3b (SEQ ID NO: 184 of U.S. Pat. No. 9,624,274), AAV7 (SEQ ID NO: 185 of U.S. Pat. No. 9,624,274), AAV8 (SEQ ID NO: 186 of U.S. Pat. No. 9,624,274), AAV10 (SEQ ID NO: 187 of U.S. Pat. No. 9,624,274), AAV4 (SEQ ID NO: 188 of U.S. Pat. No. 9,624,274), AAV11(SEQ ID NO: 189 of U.S. Pat. No. 9,624,274), bAAV (SEQ ID NO: 190 of U.S. Pat. No. 9,624,274), AAV5 (SEQ ID NO: 191 of U.S. Pat. No. 9,624,274), GPV (SEQ ID NO: 192 of U.S. Pat. No. 9,624,274; herein SEQ ID NO: 992), B19 (SEQ ID NO: 193 of U.S. Pat. No. 9,624,274; herein SEQ ID NO: 993), MVM (SEQ ID NO: 194 of U.S. Pat. No. 9,624,274; herein SEQ ID NO: 994), FPV (SEQ ID NO: 195 of U.S. Pat. No. 9,624,274, herein SEQ ID NO: 995), CPV (SEQ ID NO: 196 of U.S. Pat. No. 9,624,274; herein SEQ NO: 996) or variants thereof. Further, any of the structural protein inserts described in U.S. Pat. No. 9,624,274, may be inserted into, but not limited to, 1-453 and 1-587 of any parent AAV serotype, such as, but not limited to, AAV2 (SEQ ID NO: 183 of U.S. Pat. No. 9,624,274). The amino acid insert may be, but is not limited to, any of the following amino acid sequences, VNLTWSRASG (SEQ ID NO: 50 of U.S. Pat. No. 9,624,274; herein SEQ ID NO: 1362), EFCIINHRGYWVCGD (SEQ ID No. 55 of U.S. Pat. No. 9,624,274; herein SEQ ID NO: 1363), EDGQVMDVDLS (SEQ ID NO: 85 of U.S. Pat. No. 9,624,274; herein SEQ ID NO: 1364), EKQRNGTLT (SEQ ID NO: 86 of U.S. Pat. No. 9,624,274; herein SEQ ID NO: 1365), TYKRNITHPHLPRALMR (SEQ ID NO: 87 of U.S. Pat. No. 9,624,274; herein SEQ ID NO: 1366), RHSTTQPRKTKGSG (SEQ ID NO: 88 of U.S. Pat. No. 9,624,274; herein SEQ ID NO: 1367), DSNPRGVSAYLSR (SEQ ID NO: 89 of U.S. Pat. No. 9,624,274; herein SEQ ID NO: 1368), TITCLWDLAPSK (SEQ ID NO: 90 of U.S. Pat. No. 9,624,274; herein SEQ ID NO: 1369), KTKGSGEFVF (SEQ ID NO: 91 of U.S. Pat. No. 9,624,274; herein SEQ ID NO: 1370), THPHLPRALMRS (SEQ ID NO: 92 of U.S. Pat. No. 9,624,274; herein SEQ ID NO: 1371), GETYKRVIEIPHLPRALMRSTIK (SEQ ID NO: 93 of U.S. Pat. No. 9624274, herein SEQ ID NO: 1372), LPRALMRS (SEQ ID NO: 94 of U.S. Pat. No. 9,624,274; herein SEQ ID NO: 1373), INHRGYWV (SEQ ID NO: 95 of U.S. Pat. No. 9,624,274; herein SEQ ID NO: 1374), CDAGSVRTNAPD (SEQ ID NO: 60 of U.S. Pat. No. 9,624,274; herein SEQ ID NO: 1375), AKAVSNLTESRSESLQS (SEQ ID NO: 96 of U.S. Pat. No. 9,624,274; herein SEQ ID NO: 1376), SLTGDEFKKVLET (SEQ ID NO: 97 of U.S. Pat. No. 9,624,274; herein SEQ ID NO: 1377), REAVAYRFEED (SEQ ID NO: 98 of U.S. Pat. No. 9,624,274; herein SEQ ID NO: 1378). INPEIFFLDG (SEQ ID NO: 99 of U.S. Pat. No. 9,624,274; herein SEQ NO: 1379), DISVIGAPVITATYL (SEQ ID NO: 100 of U.S. Pat. No. 9,624,274; herein SEQ ID NO: 1380), DISVTGAPVITA (SEQ ID NO: 101 of U.S. Pat. No. 9,624,274; herein SEQ ID NO: 1381), PKTVSNLTESSSESVQS (SEQ ID NO: 102 of U.S. Pat. No. 9,624,274; herein SEQ ID NO: 1382), SLNIGDEFICWLET (SEQ ID NO: 103 of U.S. Pat. No. 9,624,274; herein SEQ ID NO: 1383), QHSVAYTFEED (SEQ ID NO: 104 of U.S. Pat. No. 9,624,274; herein SEQ ID NO: 1384), INPEIITRDG (SEQ ID NO: 105 of U.S. Pat. No. 9,624,274; herein SEQ ID NO: 1385), DISLTGDPVITASYL (SEQ ID NO: 106 of U.S. Pat. No. 9,624,274; herein SEQ ID NO: 1386), DISLIGDPVITA (SEQ ID NO: 107 of U.S. Pat. No. 9,624,274, herein SEQ ID NO: 1387). DQSIDFEIDSA (SEQ ID NO: 108 of U.S. Pat. No. 9,624,274; herein SEQ ID NO: 1388), KNVSEDLPLPTFSPTLLGDS (SEQ ID NO: 109 of U.S. Pat. No. 9,624,274; herein SEQ ID NO: 1389), KNVSEDLPLPT (SEQ ID NO: 110 of U.S. Pat. No. 9,624,274; herein SEQ ID NO: 1390), CDSGRVRTDAPD (SEQ ID NO: 111 of U.S. Pat. No. 9,624,274; herein SEQ ID NO: 1391). FPEFILINDELQSLS (SEQ ID NO: 112 of U.S. Pat. No. 9,624,274; herein SEQ ID NO: 1392), DAEFRHDSG (SEQ ID NO: 65 of U.S. Pat. No. 9,624,274; herein SEQ ID NO: 1393), HYAAAQWDFGNTMCQL (SEQ ID NO: 113 of U.S. Pat. No. 9,624,274; herein SEQ ID NO: 1394), YAAQWDFGNTMCQ (SEQ ID NO: 114 of U.S. Pat. No. 9,624,274; herein SEQ ID NO: 1395), RSQKEGLHYT (SEQ ID NO: 115 of U.S. Pat. No. 9,624,274; herein SEQ ID NO: 1396), SSRTPSDKIWAHWANPQAE (SEQ ID NO: 116 of U.S. Pat. No. 9,624,274; herein SEQ ID NO: 1397), SRTPSDKINAHWANP (SEQ ID NO: 117 of U.S. Pat. No. 9,624,274; herein SEQ ID NO: 1398), SSRTPSDKP (SEQ ID NO: 118 of U.S. Pat. No. 9,624,274; herein SEQ ID NO: 1399), NADGNVDYHMNSVP (SEQ ID NO: 119 of U.S. Pat. No. 9,624,274; herein SEQ ID NO: 1400), DGNVDYHMNSV (SEQ ID NO: 120 of U.S. Pat. No. 9,624,274; herein SEQ ID NO: 1401), RSFKEFLQSSLRALRQ (SEQ ID NO: 121 of U.S. Pat. No. 9,624,274; herein SEQ ID NO: 1402); FKEFLQSSLRA (SEQ ID NO: 122 of U.S. Pat. No. 9,624,274; herein SEQ ID NO: 1403), or QMWAPQWGPD (SEQ ID NO: 123 of U.S. Pat. No. 9,624,274; herein SEQ ID NO: 1404).

In certain embodiments, the AAV serotype may be, or may have a sequence as described in U.S. Pat. No. 9,475,845 (the content of which is incorporated herein by reference in its entirety as related to AAV capsids, insofar as it does not conflict with the present disclosure), such as, but not limited to, AAV capsid proteins comprising modification of one or more amino acids at amino acid positions 585 to 590 of the native AAV2 capsid protein. Further the modification may result in, but not limited to, the amino acid sequence RGNRQA (SEQ ID NO: 3 of U.S. Pat. No. 9,475,845; herein SEQ ID NO: 1405), SSSTDP (SEQ ID NO: 4 of U.S. Pat. No. 9,475,845, herein SEQ ID NO: 1406), SSNTAP (SEQ ID NO: 5 of U.S. Pat. No. 9,475,845; herein SEQ ID NO: 1407), SNSNLP (SEQ ID NO: 6 of U.S. Pat. No. 9,475,845; herein SEQ ID NO: 1408), SSTTAP (SEQ ID NO: 7 of U.S. Pat. No. 9,475,845; herein SEQ ID NO: 1409), AANTAA (SEQ ID NO: 8 of U.S. Pat. No. 9,475,845; herein SEQ ID NO: 1410), QQNTAP (SEQ ID NO: 9 of U.S. Pat. No. 9,475,845; herein SEQ ID NO: 1411), SAQAQA (SEQ ID NO: 10 of U.S. Pat. No. 9,475,845; herein SEQ ID NO: 1412), QANTGP (SEQ NO: 11 of U.S. Pat. No. 9,475,845; herein SEQ ID NO: 1413), NATTAP (SEQ ID NO: 12 of U.S. Pat. No. 9,475,845; herein SEQ ID NO: 1414), SSTAGP (SEQ ID NO: 13 and 20 of U.S. Pat. No. 9,475,845; herein SEQ ID NO: 1415), QQNTAA (SEQ ID NO: 14 of U.S. Pat. No. 9,475,845; herein SEQ ID NO: 1416). PSTAGP (SEQ ID NO: 15 of U.S. Pat. No. 9,475,845; herein SEQ ID NO: 1417), NQNTAP (SEQ ID NO: 16 of U.S. Pat. No. 9,475,845; herein SEQ ID NO: 1418), QAANAP (SEQ ID NO: 17 of U.S. Pat. No. 9,475,845; herein SEQ ID NO: 1419), SIVGLP (SEQ ID NO: 18 of U.S. Pat. No. 9,475,845; herein SEQ ID NO: 1420), AASTAA (SEQ ID NO: 19, and 27 of U.S. Pat. No. 9,475,845; herein SEQ ID NO: 1421), SQNTTA. (SEQ ID NO: 21 of U.S. Pat. No. 9,475,845; herein SEQ ID NO: 1422), QQDTAP (SEQ ID NO: 22 of U.S. Pat. No. 9,475,845; herein SEQ ID NO: 1423), QTNTGP (SEQ ID NO: 23 of U.S. Pat. No. 9,475,845; herein SEQ ID NO: 1424), QTNGAP (SEQ ID NO: 24 of U.S. Pat. No. 9,475,845; herein SEQ ID NO: 1425), QQNAAP (SEQ ID NO: 25 of U.S. Pat. No. 9,475,845; herein SEQ ID NO: 1426), or AANTQA (SEQ ID NO: 26 of U.S. Pat. No. 9,475,845; herein SEQ ID NO: 1427). In certain embodiments, the amino acid modification is a substitution at amino acid positions 262 through 265 in the native AAV2 capsid protein or the corresponding position in the capsid protein of another AAV with a targeting; sequence. The targeting sequence may be, but is not limited to, any of the amino acid sequences NGRAHA (SEQ ID NO: 38 of U.S. Pat. No. 9,475,845; herein SEQ ID NO: 1428), QPEHSST (SEQ ID NO: 39 and 50 of U.S. Pat. No. 9,475,845, herein SEQ ID NO: 1429), VNTANST (SEQ ID NO: 40 of U.S. Pat. No. 9,475,845; herein SEQ NO: 1430), HGPMQKS (SEQ ID NO: 41 of U.S. Pat. No. 9,475,845; herein SEQ ID NO: 1431), PHKPPLA (SEQ ID NO: 42 of U.S. Pat. No. 9,475,845; herein SEQ ID NO: 1432), IKNNEMW (SEQ ID NO: 43 of U.S. Pat. No. 9,475,845; herein SEQ ID NO: 1433), RNLDTPM (SEQ ID NO: 44 of U.S. Pat. No. 9,475,845; herein SEQ ID NO: 1434), VDSHRQS (SEQ ID NO: 45 of U.S. Pat. No. 9,475,845; herein SEQ ID NO: 1435), YDSKIKT (SEQ NO: 46 of U.S. Pat. No. 9,475,845; herein SEQ ID NO: 1436), SQLPHQK (SEQ ID NO: 47 of U.S. Pat. No. 9,475,845; herein SEQ ID NO: 1437), STMQQNT (SEQ ID NO: 48 of U.S. Pat. No. 9,475,845; herein SEQ ID NO: 1438). TERYMTQ (SEQ ID NO: 49 of U.S. Pat. No. 9,475,845; herein SEQ ID NO: 1439), DASLSTS (SEQ ID NO: 51 of U.S. Pat. No. 9,475,845; herein SEQ ID NO: 1440), DLPNKKT (SEQ ID NO: 52 of U.S. Pat. No. 9,475,845; herein SEQ ID NO: 1441), DLTAARL (SEQ ID NO: 53 of U.S. Pat. No. 9,475,845; herein SEQ ID NO: 1442), EPHQFNY (SEQ ID NO: 54 of U.S. Pat. No. 9,475,845; herein SEQ ID NO: 1443), EPQSNHT (SEQ ID NO: 55 of U.S. Pat. No. 9,475,845; herein SEQ ID NO: 1444), MSSWPSQ (SEQ ID NO: 56 of U.S. Pat. No. 9,475,845; herein SEQ ID NO: 1445), NPKHNAT (SEQ ID NO: 57 of U.S. Pat. No. 9,475,845; herein SEQ ID NO: 1446), PDGMRTT (SEQ ID NO: 58 of U.S. Pat. No. 9,475,845; herein SEQ ID NO: 1447), PNNNKTT (SEQ ID NO: 59 of U.S. Pat. No. 9,475,845; herein SEQ ID NO: 1448), QSTTHDS (SEQ ID NO: 60 of U.S. Pat. No. 9,475,845; herein SEQ ID NO: 1449), TGSKQKQ (SEQ ID NO: 61 of U.S. Pat. No. 9,475,845; herein SEQ ID NO: 1450), SLKHQAL (SEQ ID NO: 62 of U.S. Pat. No. 9,475,845; herein SEQ ID NO: 1451), SPIDGEQ (SEQ ID NO: 63 of U.S. Pat. No. 9,475,845; herein SEQ ID NO: 1452), WIFPWIQL (SEQ ID NO: 64 and 112 of U.S. Pat. No. 9,475,845; herein SEQ ID NO: 1453), CDCRGDCFC (SEQ ID NO: 65 of U.S. Pat. No. 9,475,845; herein SEQ ID NO: 1454), CNGRC (SEQ ID NO: 66 of U.S. Pat. No. 9,475,845; herein SEQ ID NO: 1455), CPRECES (SEQ ID NO: 67 of U.S. Pat. No. 9,475,845; herein SEQ ID NO: 1456), CTTHWGFTLC (SEQ ID NO: 68 and 123 of U.S. Pat. No. 9,475,845; herein SEQ ID NO: 1457), CGRRAGGSC (SEQ ID NO: 69 of U.S. Pat. No. 9,475,845; herein SEQ ID NO: 1458). CKGGRAKDC (SEQ ID NO: 70 of U.S. Pat. No. 9,475,845; herein SEQ ID NO: 1459), CVPELGHEC (SEQ ID NO: 71 and 115 of U.S. Pat. No. 9,475,845; herein SEQ ID NO: 1460), CRRETAWAK (SEQ ID NO: 72 of U.S. Pat. No. 9,475,845; herein SEQ ID NO: 1461), VSWFSHRYSPFAVS (SEQ ID NO: 73 of U.S. Pat. No. 9,475,845; herein SEQ ID NO: 1462), GYRDGYAGPILYN (SEQ ID NO: 74 of U.S. Pat. No. 9,475,845; herein SEQ ID NO: 1463), XXXYXXX (SEQ ID NO: 75 of U.S. Pat. No. 9,475,845; herein SEQ ID NO: 1464), YXNW (SEQ ID NO: 76 of U.S. Pat. No. 9,475,845; herein SEQ ID NO: 1465), RPLPPLP (SEQ ID NO: 77 of U.S. Pat. No. 9,475,845; herein SEQ ID NO: 1466), APPLPPR (SEQ NO: 78 of U.S. Pat. No. 9,475,845; herein SEQ ID NO: 1467), DVFYPYPYASGS (SEQ ID NO: 79 of U.S. Pat. No. 9,475,845; herein SEQ ID NO: 1468), MYWYPY (SEQ ID NO: 80 of U.S. Pat. No. 9,475,845; herein SEQ ID NO: 1469), DITWDQLWDLMK (SEQ ID NO: 81 of U.S. Pat. No. 9,475,845; herein SEQ ID NO: 1470), CWDDXWLC (SEQ ID NO: 82 of U.S. Pat. No. 9,475,845; herein SEQ ID NO: 1471), EWCEYLGGYLRCYA (SEQ ID NO: 83 of U.S. Pat. No. 9,475,845; herein SEQ ID NO: 1472), YXCXXGPXTWXCXP (SEQ ID NO: 84 of U.S. Pat. No. 9,475,845; herein SEQ ID NO: 1473), IEGPTLRQWLAARA (SEQ ID NO: 85 of U.S. Pat. No. 9,475,845; herein SEQ ID NO: 1474), LWXXX (SEQ ID NO: 86 of U.S. Pat. No. 9,475,845; herein SEQ NO: 1475), XFXXY LW (SEQ ID NO: 87 of U.S. Pat. No. 9,475,845; herein SEQ ID NO: 1476), SSIISHFRWGLCD (SEQ ID NO: 88 of U.S. Pat. No. 9,475,845; herein SEQ ID NO: 1477), MSRPACPPNDKYE (SEQ ID NO: 89 of U.S. Pat. No. 9,475,845; herein SEQ ID NO: 1478), CLRSGRGC (SEQ ID NO: 90 of U.S. Pat. No. 9,475,845; herein SEQ ID NO: 1479), CHWMFSPWC (SEQ ID NO: 91 of U.S. Pat. No. 9,475,845; herein SEQ ID NO: 1480), WXXF (SEQ ID NO: 92 of U.S. Pat. No. 9,475,845; herein SEQ ID NO: 1481), CSSRLDAC (SEQ ID NO: 93 of U.S. Pat. No. 9,475,845; herein SEQ ID NO: 1482), CLPVASC (SEQ ID NO: 94 of U.S. Pat. No. 9,475,845; herein SEQ ID NO: 1483). CGFECVRQCPERC (SEQ NO: 95 of U.S. Pat. No. 9,475,845; herein SEQ ID NO: 1484), CVALCREACGEGC (SEQ ID NO: 96 of U.S. Pat. No. 9,475,845; herein SEQ ID NO: 1485), SWCEPGWCR (SEQ ID NO: 97 of U.S. Pat. No. 9,475,845; herein SEQ ID NO: 1486), YSGKWGW (SEQ ID NO: 98 of U.S. 9,475,845; herein SEQ ID NO: 1487), GLSGGRS (SEQ ID NO: 99 of U.S. Pat. No. 9,475,845; herein SEQ ID NO: 1488), LMLPRAD (SEQ ID NO: 100 of U.S. Pat. No. 9,475,845; herein SEQ ID NO: 1489), CSCFRDVCC (SEQ ID NO: 101 of U.S. Pat. No. 9,475,845; herein SEQ ID NO: 1490), CRDVVSVIC (SEQ ID NO: 102 of U.S. Pat. No. 9,475,845; herein SEQ NO: 1491), MARSGL (SEQ ID NO: 103 of U.S. Pat. No. 9,475,845; herein SEQ ID NO: 1492), MARAKE (SEQ ID NO: 104 of U.S. Pat. No. 9,475,845; herein SEQ ID NO: 1493), MSRTMS (SEQ ID NO: 105 of U.S. Pat. No. 9,475,845; herein SEQ ID NO: 1494), KCCYSL (SEQ ID NO: 106 of U.S. Pat. No. 9,475,845; herein SEQ ID NO: 1495). MYWGDSHWLQYWYE (SEQ ID NO: 107 of U.S. Pat. No. 9,475,845; herein SEQ ID NO: 1496), MQLPLAT (SEQ ID NO: 108 of U.S. Pat. No. 9,475,845; herein SEQ ID NO: .1497), EWLS (SEQ ID NO: 109 of U.S. Pat. No. 9,475,845; herein SEQ ID NO: 1498), SNEW (SEQ ID NO: 110 of U.S. Pat. No. 9,475,845; herein SEQ ID NO: 1499), TNYL (SEQ ID NO: 111 of U.S. Pat. No. 9,475,845; herein SEQ ID NO: 1500), WDILAWMERLPVG (SEQ ID NO: 113 of U.S. Pat. No. 9,475,845; herein SEQ ID NO: 1501). CTVALPGGYVRVC (SEQ ID NO: 114 of U.S. Pat. No. 9,475,845; herein SEQ ID NO: 1502), CVAYCIEHHCWIC (SEQ ID NO: 116 of U.S. Pat. No. 9,475,845; herein SEQ ID NO: 1503), CVFAHNYDYLVC (SEQ ID NO: 117 of U.S. Pat. No. 9,475,845; herein SEQ ID NO: 1504), CVFTSNYAFC (SEQ NO: 118 of U.S. Pat. No. 9,475,845; herein SEQ ID NO: 1505), VHSPNKK (SEQ ID NO: 119 of U.S. Pat. No. 9,475,845; herein SEQ ID NO: 1506), CRGDGWC (SEQ ID NO: 120 of U.S. Pat. No. 9,475,845; herein SEQ ID NO: 1507), XRGCDX (SEQ ID NO: 121 of U.S. Pat. No. 9,475,845; herein SEQ ID NO: 1508), PXXX (SEQ ID NO: 122 of U.S. Pat. No. 9,475,845; herein SEQ ID NO: 1509), SGKGPRQITAL (SEQ NO: 124 of U.S. Pat. No. 9,475,845; herein SEQ ID NO: 1510), AAAAAAAAAXXXXX (SEQ ID NO: 125 of U.S. Pat. No. 9,475,845; herein SEQ ID NO: 1511), VYMSPF (SEQ ID NO: 126 of U.S. Pat. No. 9,475,845; herein SEQ ID NO: 1512), ATWLPPR (SEQ ID NO: 127 of U.S. Pat. No. 9,475,845; herein SEQ ID NO: 1513), HIMYYHEIIQHEL (SEQ ID NO: 128 of U.S. Pat. No. 9,475,845; herein SEQ ID NO: 1514), SEVGCRAGPLQWLCEKYFG (SEQ ID NO: 129 of U.S. Pat. No. 9,475,845; herein SEQ ID NO: 1515), CGLLPVGRPDRNVWRWLC (SEQ ID NO: 130 of U.S. Pat. No. 9,475,845; herein SEQ ID NO: 1516), CKGQCDRFKGLPWEC (SEQ ID NO: 131 of U.S. Pat. No. 9,475,845; herein SEQ ID NO: 1517), SGRSA (SEQ ID NO: 132 of U.S. Pat. No. 9,475,845; herein SEQ ID NO: 1518), WGFP (SEQ ID NO: 133 of U.S. Pat. No. 9,475,845; herein SEQ ID NO: 1519), AEPMPFISLNFSQYLWYT (SEQ ID NO: 134 of U.S. Pat. No. 9,475,845; herein SEQ ID NO: 1520), WAYXSP (SEQ ID NO: 135 of U.S. Pat. No. 9,475,845; herein SEQ ID NO: 1521), IELLQAR (SEQ ID NO: 136 of U.S. Pat. No. 9,475,845; herein SEQ ID NO: 1522), AYTKCSRQWRTCMTTII (SEQ ID NO: 137 of U.S. Pat. No. 9,475,845; herein SEQ ID NO: 1523), PQNSKIPGPTFLDPH (SEQ ID NO: 138 of U.S. Pat. No. 9,475,845; herein SEQ ID NO: 1524), SMEPALPDWWWKNIFK (SEQ ID NO: 139 of U.S. Pat. No. 9,475,845; herein SEQ ID NO: 1525), NNTPCGPYTHDCPVKR (SEQ ID NO: 140 of U.S. Pat. No. 9,475,845; herein SEQ ID NO: 1526), TACHQFIVRNIVRP (SEQ ID NO: 141 of U.S. Pat. No. 9,475,845; herein SEQ ID NO: 1527), VPWMEPAYQRFL (SEQ ID NO: 142 of U.S. Pat. No. 9,475,845; herein SEQ ID NO: 1528), DPRATPGS (SEQ ID NO: 143 of U.S. Pat. No. 9,475,845; herein SEQ ID NO: 1529), FRPNRAQDYNTN (SEQ ID NO: 144 of U.S. Pat. No. 9,475,845; herein SEQ ID NO: 1530), CTKNSYLMC (SEQ ID NO: 145 of U.S. Pat. No. 9,475,845; herein SEQ ID NO: 1531), CXXTXXXGXGC (SEQ ID NO: 146 of U.S. Pat. No. 9,475,845; herein SEQ ID NO: 1532), CPIEDRPMC (SEQ ID NO: 147 of U.S. Pat. No. 9,475,845; herein SEQ ID NO: 1533), FIEWSYLAPYPWF (SEQ ID NO: 148 of U.S. Pat. No. 9,475,845; herein SEQ ID NO: 1534). MCPKEPLGC (SEQ ID NO: 149 of U.S. Pat. N9o. 9,475,845; herein SEQ ID NO: 1535), RMWPSSTVNLSAGRR (SEQ ID NO: 150 of U.S. Pat. No. 9,475,845; herein SEQ ID NO: 1536), SAKTAVSQRVWLPSFIRGGEP (SEQ ID NO: 151 of U.S. Pat. No. 9,475,845; herein SEQ ID NO: 1537), KSREIIVNNSACPSKRITAAL (SEQ ID NO: 152 of U.S. Pat. No. 9,475,845; herein SEQ ID NO: 1538), EGFR (SEQ ID NO: 153 of U.S. Pat. No. 9,475,845; herein SEQ ID NO: 1539), AGLGVR (SEQ IL) NO: 154 of U.S. Pat. No. 9,475,845; herein SEQ ID NO: 1540), GTRQGHTMRLGVSDG (SEQ ID NO: 155 of 059475845; herein SEQ ID NO: 1541), IAGLATPGWSHWLAL (SEQ ID NO: 156 of U.S. Pat. No. 9,475,845; herein SEQ ID NO: 1542), SMSIARL (SEQ ID NO: 157 of U.S. Pat. No. 9,475,845; herein SEQ ID NO: 1543), HTFEPGV (SEQ ID NO: 158 of U.S. Pat. No. 9,475,845; herein SEQ ID NO: 1544), NTSLKRISNKRIRRK (SEQ ID NO: 159 of U.S. Pat. No. 9,475,845; herein SEQ ID NO: 1545), LRIKRKRRKRKKTRK (SEQ ID NO: 160 of U.S. Pat. No. 9,475,845; herein SEQ ID NO: 1546), GGG, GFS, DVS, EGG, LLV, LSP, LBS, AGG, GRR, GGH, or GTV.

In certain embodiments, the AAV serotype may be, or may have a sequence as described in U.S. Patent Application Publication No. US 20160369298 (the content of which is incorporated herein by reference in its entirety as related to AAV capsids, insofar as it does not conflict with the present disclosure), such as, but not limited to, site-specific mutated capsid protein of AAV2 (SEQ ID NO: 97 of US 20160369298; herein SEQ ID NO: 1547) or variants thereof, wherein the specific mutated site is at least one site selected from sites R447, G453, 5578, N587, N587+1, S662 of VP1 or fragment thereof.

Further, any of the mutated sequences described in US 20160369298, may be or may have, but not limited to, any of the following; sequences: SDSGASN (SEQ ID NO: 1 and. SEQ ID NO: 231 of US20160369298; herein SEQ m NO: 1548), SPSGASN (SEQ ID NO: 2 of US20160369298; herein SEQ ID NO: 1549), SHSGASN (SEQ :113 NO: 3 of US20160369298; herein SEQ ID NO: 1550), SRSGASN (SEQ IL) NO: 4 of US20160369298, herein SEQ ID NO: 1551), SKSGASN (SEQ ID NO: 5 of US20160369298; herein SEQ ID NO: 1552), SNSGASN (SEQ ID NO: 6 of US20160369298; herein SEQ ID NO: 1553), SGSGASN (SEQ ID NO: 7 of US20160369298; herein SEQ ID NO: 1554), SASGASN (SEQ ID NO: 8, 175, and 221 of US20160369298; herein SEQ ID NO: 1555), SESGTSN (SEQ ID NO: 9 of US20160369298; herein SEQ ID NO: 1556), STTGGSN (SEQ ID NO: 10 of US20160369298; herein SEQ ID NO: 1557), SSAGSTN (SEQ ID NO: 11 of US20160369298; herein SEQ ID NO: 1558), NNDSQA (SEQ ID NO: 12 of US20160369298; herein SEQ ID NO: 1559), NNRNQA (SEQ ID NO: 13 of US20160369298; herein SEQ ID NO: 1560), NNNKQA (SEQ ID NO: 14 of US20160369298; herein SEQ ID NO: 1561), NAKRQA (SEQ ID NO: 15 of US20160369298; herein SEQ ID NO: 1562), NDEHQA (SEQ ID NO: 16 of US20160369298; herein SEQ ID NO: 1563), NTSQKA (SEQ ID NO: 17 of US20160369298; herein SEQ ID NO: 1564), YYLSRTNTPSGTDIQSRLVESQAGA (SEQ ID NO: 18 of US20160369298; herein SEQ ID NO: 1565), YYLSRTNTDSGTETQSGLDFSQAGA (SEQ ID NO: 19 of US20160369298; herein SEQ ID NO: 1566), YYLSRTNTESGTPTQSALEFSQAGA (SEQ ID NO: 20 of US20160369298; herein SEQ ID NO: 1567), YYLSRTNTHSGTHTQSPLHFSQAGA (SEQ ID NO: 21 of US20160369298; herein SEQ ID NO: 1568), YYLSRINTSSGTITISHLIFSQAGA (SEQ ID NO: 22 of US20160369298; herein SEQ ID NO: 1569), YYLSRTNTRSGIMTKSSLMFSQAGA (SEQ ID NO: 23 of US20160369298; herein SEQ ID NO: 1570), YYLSRTNTKSGRKTLSNLSFSQAGA (SEQ ID NO: 24 of US20160369298; herein SEQ ID NO: 1571), YYLSRINDGSGPVTPSKLRFSQRGA (SEQ ID NO: 25 of US20160369298; herein SEQ ID NO: 1572), YYLSRTNAASGHATHSDLKFQPGA (SEQ ID NO: 26 of US20160369298; herein SEQ ID NO: 1573), YYLSRTNGQAGSLTMSELGESQVGA (SEQ ID NO: 27 of US20160369298; herein SEQ ID NO: 1574), YYLSRTNSTGGNQTTSQLLFSQLSA (SEQ ID NO: 28 of US20160369298; herein SEQ ID NO: 1575), YFLSRTNNNTGLNTNSTLNFSQGRA (SEQ ID NO: 29 of US20160369298; herein SEQ ID NO: 1576), SKTGADNNNSEYSWTG (SEQ ID NO: 30 of US20160369298; herein SEQ ID NO: 1577), SKTDADNNNSEYSWTG (SEQ ID NO: 31 of US20160369298; herein SEQ ID NO: 1578), SKTEADN⁻NNSEYSWTG (SEQ ID NO: 32 of US20160369298; herein SEQ ID NO: 1579), SKTPADNNNSEYSWTG (SEQ ID NO: 33 of US20160369298; herein SEQ ID NO: 1580), SKTHADNNNSEYSWTG (SEQ ID NO: 34 of US20160369298; herein SEQ ID NO: 1581), SKTQADNNNSEYSWTG (SEQ ID NO: 35 of US20160369298; herein SEQ NO: 1582). SKTIADNNNSEYSWTG (SEQ 11113 NO: 36 of US20160369298; herein SEQ ID NO: 1583), SKTMADNNNSEYSWTG (SEQ ID NO: 37 of US20160369298; herein SEQ ID NO: 1584), SKTRADNNNSEYSWTG (SEQ ID NO: 38 of US20160369298; herein SEQ ID NO: 1585), SKTNADNNNSEYSWTG (SEQ ID NO: 39 of US20160369298; herein SEQ ID NO: 1586), SKTVGRNNNSEYSWTG (SEQ ID NO: 40 of US20160369298; herein SEQ ID NO: 1587), SKTADRNNNSEYSWTG (SEQ ID NO: 41 of US20160369298, herein SEQ ID NO: 1588), SKKLSQNNNSKYSWQG (SEQ ID NO: 42 of US20160369298; herein SEQ ID NO: 1589), SKPTTGNNNSDYSWPG (SEQ ID NO: 43 of US20160369298; herein SEQ ID NO: 1590), STQKNENNNSNYSWPG (SEQ ID NO: 44 of US20160369298; herein SEQ ID NO: 1591), HKDDEGKF (SEQ ID NO: 45 of US20160369298; herein SEQ ID NO: 1592), HKDDNRKF (SEQ ID NO: 46 of US20160369298; herein SEQ ID NO: 1593). FIKDDINKF (SEQ ID NO: 47 of US20160369298; herein SEQ ID NO: 1594), HEDSDKNF (SEQ ID NO: 48 of US20160369298; herein SEQ ID NO: 1595), HRDGADST (SEQ ID NO: 49 of US20160369298; herein SEQ ID NO: 1596), HGDNKSRF (SEQ ID NO: 50 of US20160369298; herein SEQ ID NO: 1597). KQGSEKTNVDFEEV (SEQ ID NO: 51 of US20160369298; herein SEQ ID NO: 1598), KQGSEKTNVDSEEV (SEQ ID NO: 52 of US20160369298; herein SEQ ID NO: 1599), KQGSEKTNVDVEEV (SEQ ID NO: 53 of US20160369298; herein SEQ ID NO: 1600), KQGSDKINVDDAGV (SEQ ID NO: 54 of US20160369298; herein SEQ ID NO: 1601), KQGSSKTNVDPREV (SEQ ID NO: 55 of US20160369298; herein SEQ ID NO: 1602), KQGSRKTNVDHKQV (SEQ ID NO: 56 of US20160369298; herein SEQ ID NO: 1603), KQGSKGGNVDTNRV (SEQ ID NO: 57 of US20160369298; herein SEQ ID NO: 1604), KQGSGEANVDNGDV (SEQ NO: 58 of US20160369298; herein SEQ ID NO: 1605), KQDAAADNIDYDHV (SEQ ID NO: 59 of US20160369298; herein SEQ ID NO: 1606), KQSGTRSNAAASSV (SEQ ID NO: 60 of US20160369298; herein SEQ ID NO: 1607), KENTNTNDTELTNV (SEQ ID NO: 61 of US20160369298; herein SEQ ID NO: 1608), QRGNNVAATADVNT (SEQ ID NO: 62 of US20160369298; herein SEQ ID NO: 1609), QRGNNEAATADVNT (SEQ ID NO: 63 of US20160369298; herein SEQ ID NO: 1610), QRGNNPAATADVNT (SEQ ID NO: 64 of US20160369298; herein SEQ ID NO: 1611), QRGNNHAATADVNT (SEQ ID NO: 65 of US20160369298; herein SEQ ID NO: 1612), QEENNIAATPGVNT (SEQ ID NO: 66 of US20160369298; herein SEQ ID NO: 1613), QPPNNIMAA IHEVNT (SEQ ID NO: 67 of US20160369298; herein SEQ ID NO: 1614), QHHNNSAATTIVNT (SEQ ID NO: 68 of US20160369298; herein SEQ ID NO: 1615), QTTNNRAAFNMVET (SEQ IL) NO: 69 of US20160369298; herein SEQ ID NO: 1616), QKKNNNAASKKVAT (SEQ ID NO: 70 of US20160369298; herein SEQ ID NO: 1617), QGGNNKAADDAVKT (SEQ ID NO: 71 of US20160369298; herein SEQ ID NO: 1618), QAAKGGAADDAVKT (SEQ ID NO: 72 of US20160369298; herein SEQ ID NO: 1619), QDDRAAAANESVDT (SEQ ID NO: 73 of US20160369298; herein SEQ ID NO: 1620), QQQHDDAAYQRVHT (SEQ ID NO: 74 of US20160369298; herein SEQ ID NO: 1621), QSSSSLAAVSTVQT (SEQ ID NO: 75 of US20160369298; herein SEQ ID NO: 1622), QNNQTTAAIRNVTT (SEQ ID NO: 76 of US20160369298; herein SEQ ID NO: 1623), NYNKKSDNVDFT (SEQ ID NO: 77 of US20160369298; herein SEQ ID NO: 1624), NYNKKSENVDFT (SEQ ID NO: 78 of US20160369298; herein SEQ ID NO: 1625), NYNKKSLNVDFT (SEQ ID NO: 79 of US20160369298; herein SEQ ID NO: 1626), NYNKKSPNVDFT (SEQ ID NO: 80 of US20160369298; herein SEQ ID NO: 1627), NYSKKSHCVDFT (SEQ ID NO: 81 of US20160369298; herein SEQ ID NO: 1628), NYRKFIYVDFT (SEQ ID NO: 82 of US20160369298; herein SEQ ID NO: 1629), NYKEKKDVHFT (SEQ ID NO: 83 of US20160369298; herein SEQ ID NO: 1630), NYGHRAIVQFT (SEQ ID NO: 84 of US20160369298; herein SEQ ID NO: 163 NYANHQFVVCT (SEQ ID NO: 85 of US20160369298; herein SEQ ID NO: 1632), NYDDDPTGVLLT (SEQ ID NO: 86 of US20160369298; herein SEQ ID NO: 1633), NYDDRIGVIAT (SEQ IL) NO: 87 of US20160369298; herein SEQ ID NO: 1634), NFEQQNSVEWT (SEQ ID NO: 88 of US20160369298; herein SEQ ID NO: 1635), SQSGASN (SEQ ID NO: 89 and SEQ ID NO: 241 of US20160369298; herein SEQ ID NO: 1636), NNGSQA (SEQ ID NO: 90 of US20160369298; herein SEQ ID NO: 1637), YYLSRTNTPSGTTTWSRLQFSQAGA (SEQ ID NO: 91 of 0520160369298; herein SEQ ID NO: 1638), SKTSADNNNSEYSWTG (SEQ ID NO: 92 of 0520160369298; herein SEQ ID NO: 1639), IIKDDEEKE (SEQ ID NO: 93, 209, 214, 219, 224, 234, 239, and 244 of US20160369298; herein SEQ ID NO: 1640), KQGSEKTNVDIEEV (SEQ ID NO: 94 of US20160369298; herein SEQ ID NO: 1641), QRGNNQAATADVNT (SEQ ID NO: 95 of US20160369298; herein SEQ ID NO: 1642), NYNKKSVNVDFT (SEQ ID NO: 96 of US20160369298; herein SEQ ID NO: 1643), SQSGASNYNTPSGTTTQSRLQFSTSADNNNSEYSWTGATKYH (SEQ ID NO: 106 of US20160369298; herein SEQ ID NO: 1644), SASGASNFNSEGGSLTQSSLGFSTDGENNNSDFSWTGATKYFI (SEQ ID NO: 107 of US20160369298; herein SEQ ID NO: 1645), SQSGASNYNTPSGTTTQSRILQFSTDGENNNSDFSWTGATKYH (SEQ ID NO: 108 of US20160369298; herein SEQ ID NO: 1646), SASGASNYNIPSGITIQSRLQESTSADNNNSEFSWPGATIYH (SEQ ID NO: 109 of US20160369298; herein SEQ ID NO: 1647), SQSGASNFNSEGGSLTQSSLGESIDGENNNSDFSWTGATKYFI (SEQ ID NO: 110 of US20160369298; herein SEQ ID NO: 1648), SASGASNYNTPSGSLTQSSLGFSTDGENNNSDFSWTGATKYH (SEQ ID NO: 111 of US20160369298; herein SEQ ID NO: 1649), SQSGASNYNTPSG11TQSRLQFSTSADNNNSDESWTGATKYFI (SEQ ID NO: 112 of US20160369298; herein SEQ ID NO: 1650), SGAGASNFNSEGGSLTQSSLGFSTDGENNNSDFSWTGATKYH (SEQ ID NO: 113 of US20160369298; herein SEQ ID NO: 1651), SGAGASN (SEQ ID NO: 176 of US20160369298; herein SEQ ID NO: 1652), NSEGGSLTQSSLGFS (SEQ ID NO: 177, 185, 193 and 202 of US20160369298; herein SEQ ID NO: 1653), TDGENNNSDFS (SEQ ID NO: 178 of US20160369298; herein SEQ ID NO: 1654), SEESWPGATT (SEQ ID NO: 179 of US20160369298; herein SEQ ID NO: 1655), TSADNNNSDFSWT (SEQ ID NO: 180 of US20160369298; herein SEQ ID NO: 1656), SQSGASNY (SEQ ID NO: 181, 187, and 198 of US20160369298; herein SEQ ID NO: 1657), NTPSGTTTQSRLQFS (SEQ ID NO: 182, 188, 191, and 199 of US20160369298; herein SEQ ID NO: 1658), TSADNNNSEYSWTGATKYII (SEQ ID NO: 183 of US20160369298; herein SEQ ID NO: 1659), SASGASNF (SEQ ID NO: 184 of US20160369298; herein SEQ ID NO: 1660), TDGENNNSDFSWTGATKYH (SEQ ID NO: 186, 189, 194, 197, and 203 of US20160369298; herein SEQ ID NO: 1661), SASGASNY (SEQ ID NO: 190 and SEQ NO: 195 of US20160369298; herein SEQ ID NO: 1662), ISADNNNSEFSWPGATTYH (SEQ ID NO: 192 of US20160369298; herein SEQ ID NO: 1663), N IPSGSLTQSSLGFS (SEQ ID NO: 196 of US20160369298; herein SEQ ID NO: 1664), TSADNNNSDFSWTGAIKYI-I (SEQ ID NO: 200 of US20160369298; herein SEQ ID NO: 1665), SGAGASNF (SEQ ID NO: 201 of US20160369298; herein SEQ ID NO: 1666), CTCCAGVNISVVSMRSRMINSGCAGCIDHONSRNSGTCVMSACACAA (SEQ ID NO: 204 of US20160369298; herein SEQ ID NO: 1667), CTCCAGAGAGGCAACAGACAAGCAGCTACCGCAGATGTCAACACACAA (SEQ ID NO: 205 of U520160369298; herein SEQ ID NO: 1668), SAAGASN (SEQ ID NO: 206 of US20160369298; herein SEQ ID NO: 1669), YFLSRTNTESGSTTQSTLRFSQAG (SEQ ID NO: 207 of US20160369298; herein SEQ ID NO: 1670), SKTSADNNNSDPS (SEQ NO: 208, 228, and 253 of US20160369298; herein SEQ ID NO: 1671), KQGSEKTDVDIDKV (SEQ ID NO: 210 of US20160369298; herein SEQ ID NO: 1672), STAGASN (SEQ ID NO: 211 of US20160369298; herein SEQ ID NO: 1673), YFISRINTISGIETQSTURFSQAG (SEQ ID NO: 212 and SEQ ID NO: 247 of US20160369298; herein SEQ ID NO: 1674), SKTDGENNNSDFS (SEQ ID NO: 213 and SEQ ID NO: 248 of US20160369298; herein SEQ ID NO: 1675), KQGAAADDVEIDGV (SEQ ID NO: 215 and SEQ ID NO: 250 of US20160369298; herein SEQ ID NO: 1676), SEAGASN (SEQ ID NO: 216 of US20160369298; herein SEQ ID NO: 1677), YYLSRTNIPSGTHQSRLQFSQAG (SEQ ID NO: 217, 232 and 242 of US20160369298; herein SEQ ID NO: 1678), SKTSADNNNSEYS (SEQ ID NO: 218, 233, 238, and 243 of US20160369298; herein SEQ ID NO: 1679), KQGSEKTNVDIEKV (SEQ ID NO: 220, 225 and 245 of US20160369298; herein SEQ ID NO: 1680), YFLSRTNDASGSDTKS ILLESQAG (SEQ ID NO: 222 of US20160369298; herein SEQ ID NO: 1681), STTPSENNNSEYS (SEQ ID NO: 223 of US20160369298, herein SEQ ID NO: 1682). SAAGATN (SEQ ID NO: 226 and. SEQ ID NO: 251 of US20160369298; herein SEQ ID NO: 1683), YFLSRINGEAGSAILSELRFSQAG (SEQ ID NO: 227 of US20160369298; herein SEQ ID NO: 1684), HGDDADRF (SEQ ID NO: 229 and SEQ ID NO: 254 of US20160369298; herein SEQ ID NO: 1685), KQGAEKSDVEVDRV (SEQ ID NO: 230 and SEQ ID NO: 255 of US20160369298; herein SEQ ID NO: 1686), KQDSGGDNIDIDQV (SEQ ID NO: 235 of US20160369298; herein SEQ ID NO: 1687), SDAGASN (SEQ ID NO: 236 of US20160369298; herein SEQ ID NO: 1688), YFLSRTNTEGGHDTQSTLIUSQAG (SEQ ID NO: 237 of US20160369298; herein SEQ ID NO: 1689), KEDGGGSDVAIDEV (SEQ ID NO: 240 of US20160369298; herein SEQ ID NO: 1690), SNAGASN (SEQ ID NO: 246 of US20160369298; herein SEQ ID NO: 1691), and NTLSRINGEAGSATLSELRFSQPG (SEQ ID NO: 252 of US20160369298; herein SEQ ID NO: 1692). Non-limiting examples of nucleotide sequences that may encode the amino acid mutated sites comprise the following, AGCVVMDCAGGARSCASCAAC (SEQ ID NO: 97 of US20160369298; herein SEQ ID NO: 1693), AACRACRRSMRSMAGGCA (SEQ ID NO: 98 of US20160369298; herein SEQ ID NO: 1694), CACRRGGACRRCRMSRRSARSTTT (SEQ ID NO: 99 of US20160369298; herein SEQ ID NO: 1695), TATTTCTTGAGCAGAACAAACRVCVVSRSCGGAMNCVHSACGMHSTCAVVSCTTV DSTITICTCAGSBCRGSGCG (SEQ ID NO: 100 of US20160369298; herein SEQ ID NO: 1696), TCAAMAMMAVNSRVCSRSAACAACAACAGTRASTTCTCGTGGMMAGGA (SEQ ID NO: 101 of US20160369298; herein SEQ ID NO: 1697), AAGSAARRCRSCRYSRVARVCRATRYCGMSNHCIWIVIVRSGTC (SEQ ID NO: 102 of US20160369298; herein SEQ ID NO: 1698), CAGVVSVVSMRSRVCVNSGCAGCTDHCVVSRNSGTCVMSACA (SEQ ID NO: 103 of US20160369298; herein SEQ ID NO: 1699), AACTWCRVSVASMATSVHSDDTGTGSWSTKSACT (SEQ ID NO: 104 of US20160369298; herein SEQ ID NO: 1700), TTG ITGAACATCACCACGTGACGCACGTTC (SEQ ID NO: 256 of US20160369298; herein SEQ ID NO: 1701), TCCCCGTGGITCTACTACATAATGTGGCCG (SEQ ID NO: 257 of US20160369298; herein SEQ ID NO: 1702), TTCCACACTCCGTTTTGGATAATGTTGAAC (SEQ ID NO: 258 of US20160369298; herein SEQ ID NO: 1703), AGGGACATCCCCAGCTCCATGCTGTGGTCG (SEQ ID NO: 259 of US20160369298; herein SEQ ID NO: 1704), AGGGACAACCCCTCCGACTCGCCCTAATCC (SEQ ID NO: 260 of US20160369298; herein SEQ ID NO: 1705). TCCTAGTAGAAGACACCCTCTCACTGCCCG (SEQ ID NO: 261 of US20160369298; herein SEQ m NO: 1706), AGTACCATGTACACCCACTCTCCCAGTGCC (SEQ ID NO: 262 of US20160369298; herein SEQ ID NO: 1707), ATATGGACGTTCATGCTGATCACCATACCG (SEQ ID NO: 263 of US20160369298; herein SEQ ID NO: 1708), AGCAGGAGCTCCTTGGCCTCAGCGTGCGAG (SEQ ID NO: 264 of US20160369298; herein SEQ ID NO: 1709). ACAAGCAGCTTCACTATGACAACCACTGAC (SEQ ID NO: 265 of US20160369298; herein SEQ ID NO: 1710), CAGCCTAGGAACTGGCTTCCTGGACCCTGGACCGCCAGCAGAGAGTCTCAAMA MMAVNSRVCSRSAACAACAACAGTRASTTCTCCTGGMMAGGAGCTACCAAGTAC CACCTCAATGGCAGAGACTCTCTGGTGAATCCCGGACCAGCTATGGCAAGCCAC RRGGACRRCRMSRRSARSTTTTTTCCTCAGAGCGGGGTTCTCATCTTTGGGAAGSA ARRCRSCRVSRVARVCRATRYCGMSNFICRVMVRSGTCATGATTACAGACGAAGA GGAGATCTGGAC (SEQ ID NO: 266 of US20160369298; herein SEQ ID NO: 1711). TGGGACAATGGCGGTCGIVICTCAGAGTTKTKKI (SEQ ID NO: :267 of US20160369298; herein SEQ ID NO: 1712), AGAGGACCKKTCCTCGATGGTTCATGGTGGAGTTA (SEQ ID NO: 268 of US20160369298; herein SEQ ID NO: 1713), CCACTTAGGGCCTGGTCGATACCGTTCGGTG (SEQ ID NO: 269 of US20160369298; herein SEQ ID NO: 1714). or TCTCGCCCCAAGAGTAGAAACCCTTCSTTYYG (SEQ ID NO: 270 of US20160369298; herein SEQ ID NO: 1715).

In certain embodiments, the AAV serotype may comprise an ocular cell targeting peptide as described in International Patent Publication WO2016134375 (the content of which is incorporated herein by reference in its entirety as related to AAV capsids, insofar as it does not conflict with the present disclosure), such as, but not limited to SEQ ID NO: 9. or SEQ ID NO:10 of WO2016134375. Further, any of the ocular cell targeting peptides or amino acids described in WO2016134375, may be inserted into any parent AAV serotype, such as, but not limited to, AAV2 (SEQ ID NO:8 of WO2016134375; herein SEQ ID ⁻NO: 1716), or AAV9 (SEQ ID NO: 11 of WO2016134375; herein SEQ ID NO: 1717). In certain embodiments, modifications, such as insertions are made in AAV2 proteins at P34-A35, T138-A139, A139-P140, 0453-T454, N587-R588, and/or R588-Q589. In certain embodiments, insertions are made at D384, G385, 1560, T561, N562, E563, E564, E565, N704, and/or Y705 of AAV9. The ocular cell targeting peptide may be, but is not limited to, any of the following amino acid sequences, GSTPPPM (SEQ ID NO: 1 of WO2016134375; herein SEQ m NO: 1718), or GETRAPL (SEQ ID NO: 4 of WO2016134375; herein SEQ ID NO: 1719).

In certain embodiments, the AAV serotype may be modified as described in U.S. Patent Application Publication No, US 20170145405 (the content of which is incorporated herein by reference in its entirety as related to AAV capsids, insofar as it does not conflict with the present disclosure). AAV serotypes may comprise, modified AAV2 (e.g., modifications at Y444F, Y500F, Y730F and/or S662V), modified AAV3 (e.g., modifications at Y705F, Y731F and/or T492V), and modified AAV6 (e.g., modifications at S663V and/or T492V).

In certain embodiments, the AAV serotype may be modified as described in the International Publication No. WO2017083722 (the content of which is incorporated herein by reference in its entirety as related to AAV capsids, insofar as it does not conflict with the present disclosure). AAV serotypes may comprise, AAV1 (Y705+731F+T492V), AAV2 (Y444+500+730F+T491V), AAV3 (Y705+73 IF), AAV5, AAV 5(Y436+693+719F), AAV6 (VP3 variant Y705F/Y731.17T492V) AAV8 (Y733F), AAV9, AAV9 (VP3 variant Y731F), and AAV10 (Y733F).

In certain embodiments, the AAV serotype may comprise, as described in International Patent Publication No. WO2017015102 (the content of which is incorporated herein by reference in its entirety as related to AAV capsids, insofar as it does not conflict with the present disclosure), an engineered epitope comprising the amino acids SPAKFA (SEQ ID NO: 24 of WO2017015102; herein SEQ ID NO: 1720) or NKDKILN (SEQ NO:2 of WO2017015102; herein SEQ ID NO: 1721). The epitope may be inserted in the region of amino acids 665 to 670 based on the numbering of the VP1 capsid of AAV8 (SEQ ID NO: 3 of WO2017015102) and/or residues 664 to 668 of AAV3B (SEQ ID NO: 3).

In certain embodiments, the AAV serotype may be, or may have a sequence as described in International Patent Publication No. WO 017058892 (the content of which is incorporated herein by reference in its entirety as related to AAV capsids, insofar as it does not conflict with the present disclosure), such as, but not limited to, AAV variants with capsid proteins that may comprise a substitution at one or more (e.g., 2, 3, 4, 5, 6, or 7) of amino acid residues 262-268, 370-379, 451-459, 472-473, 493-500, 528-534, 547-552, 588-597, 709-710, or 716-722 of AAV1, in any combination, or the equivalent amino acid residues in AAV2, AAV3, AAV4, AAV5, AAV6, AAV7, AAV8, AAV9, AAV 10, AAV11, AAV12, AAVrh8, AAVrh10, AAVrh32.33, bovine AAV, or avian AAV. The amino acid substitution(s) may be, but is/are not. limited to, any of the amino acid sequences described in WO2017058892. In certain embodiments, the AAV may rise an amino acid substitution at residues 256L, 258K, 259Q, 261S, 263A, 264S, 265T, 266G, 272H, 385S, 386Q, S472R, V473D, N500E 547S, 709A, 710N, 716D, 717N, 718N, 720I- A456T, Q457T, N458Q, K459S, T492S, K493A, S586R, S587G, S588N, T589R and/or 722T of AAV1 (SEQ ID NO: 1 of WO2017058892) in any combination, 244N, 246Q, 248R, 249E, 250I, 251K, 252S, 253G, 254S, 255V, 256D, 263Y, 377E, 378N, 453L, 456R, 532Q, 533P, 535N, 536P, 537G, 538T, 539T, 540A, 541T, 542Y, 543L, 546N, 653V, 654P, 656S, 697Q, 698F, 704D, 705S, 706T, 707G, 708E, 709Y and/or 710R of AAV5 (SEQ ID NO:5 of WO2017058892) in any combination, 248R, 316V, 317Q, 318D, 319S, 443N, 530N, 531S, 532Q, 533P, 534A, 535N, 540A, 541 T, 542Y, 543L, 545G, 546N, 697Q, 704D, 706T, 708E, 709Y and/or 710R of AAVS (SEQ ID NO: 5 of WO2017058892) in any combination, 264S, 266G, 269N, 272H, 457Q, 588S and/or 589I of AAV6 (SEQ ID NO:6 WO2017058892) in any combination, 457I, 459N, 496G, 499N, 500N, 589Q, 590N and/or 592A of AAV8 (SEQ ID NO: 8 WO2017058892) in any combination,451I, 452N, 453G, 454S, 455G, 456Q, 457N and/or 458Q of AAV9 (SEQ ID NO: 9 WO2017058892) in any combination.

In certain embodiments, the AAV may comprise a sequence of amino acids at positions 155, 156, and 157 of VP1 or at positions 17, 18, 19, and 20 of VP2, as described in International Publication No. WO 2017066764 (the content of which is incorporated herein by reference in its entirety as related to AAV capsids, insofar as it does not conflict with the present disclosure). The sequences of amino acid may be, but are not limited to, N-S-S, S-X-S, S-S-Y, N-X-S, N-S-Y, S-X-Y, or N-X-Y, where N, X, and Y are, but not limited to, independently, non-serine or non-threonine amino acids, wherein the AAV may be, but is not limited to, AAV1, AAV2, AAV3, AAV4, AAV5, AAV6, AAV7, AAV8, AAV9, AAV10, AAV11, or AAV12. In certain embodiments, the AAV may comprise a deletion of at least one amino acid at position(s) 156, 157, or 158 of VP1 or at positions 19, 20, or 21 of VP2, wherein the AAV may be, but is not limited to AAVI, AAV2, AAV3, AAV4, AAVS, AAV6, AAV7, AAV8, AAV9, AAV10, AAV 11, or AAV12.

In certain embodiments, the AAV may be a serotype generated by Crc-recombination-based AAV targeted evolution (CREATE) as described by Deverman et al., (Nature Biotechnology 34(2):204-209 (2016)), Chan et al., (Nature Neuroscience 20(8):1172-1179 (2017)), and in International Patent Application Publication Nos. WO2015038958 and WO2017100671 (the content of which is incorporated herein by reference in its entirety as related to AAV capsids, insofar as it does nor. conflict with the present disclosure). In certain embodiments, AAV serotypes generated in this mariner have improved CNS transduction and/or neuronal and astrocytic tropism, as compared to AAV serotypes not generated in this manner. As non-limiting examples, the AAV serotype may comprise a targeting peptide such as, but not limited to, PHP.B, PHP.B2, PHP.B3, PHP.A, PHP.S, PHP.N, G2A12, G2A15, G2A3, G2B4, or G2B5. In certain embodiments, these AAV serotypes may be derivates of AAV9 (SEQ ID NO: 136) or AAV9 K449R (SEQ 11) NO: 9) with an amino acid insert between amino acids 588 and 589. Non-limiting examples of these amino acid inserts comprise TLAVPFK SEQ ID NO: 1260), SVSKPFL (PHP.B2, SEQ ID NO: 1268), FTLTTPK (PHP.B3; SEQ ID NO: 1269), YTLSQGW (PHP.A; SEQ ID NO: 1275), QAVRTSL (PHP.S; SEQ ID NO: 1319), LAKERLS (G2A3; SEQ ID NO: 1320), MNSTKNV (G2B4; SEQ ID NO: 1321), VSGGHHS (G2B5; SEQ ID NO: 1322), and/or DGTLAVPFKAQ (PHP.N; SEQ ID NO: 1289).

In certain embodiments, the AAV serotype may be as described in Jackson et al (Frontiers in Molecular Neuroscience 9:154 (2016)) (the content of which is incorporated herein by reference in its entirety as related to AAV capsids, insofar as it does not conflict with the present disclosure).

In certain embodiments, the AAV serotype is AAV9 (SEQ ID NO: 135 or 136). In certain embodiments, the AAV serotype is an AAV9 with a peptide insert.

In certain embodiments, the AAV serotype is a K449R AAV9 variant (SEQ ID NO: 9). AAV9 K449R has the same function as wild-type A.A.V9. In certain embodiments, the AAV serotype is an AAV9 K449R, with a peptide insert.

In certain embodiments, the AAV serotype is PHP.B (e.g., as described in WO2015038958). In certain embodiments, the AAV serotype is paired with a synapsin promoter to enhance neuronal transduction, as compared to when more ubiquitous promoters are used (i.e., CBA or CMV).

In certain embodiments, the AAV serotype is PHP.N (e.g., as described in WO2017100671).

In certain embodiments, the AAV serotype is a serotype comprising the AAVPHP.N (PHP.N) peptide or a variant thereof.

In certain embodiments, the AAV serotype is a serotype comprising the AAVPHP.B (PHP.B) peptide or a variant thereof.

In certain embodiments, the AAV serotype is a serotype comprising the AAVPHP.A (PHRA) peptide or a variant thereof

In certain embodiments, the AAV serotype is a serotype comprising the PHP.S peptide or a variant thereof.

In certain embodiments, the AAV serotype is a serotype comprising the PHP.B2 peptide or a variant thereof.

In certain embodiments, the AAV serotype is a serotype comprising the PHP.B3 peptide or a variant thereof.

In certain embodiments, the AAV serotype is a serotype comprising the G2B4 peptide or a variant thereof.

In certain embodiments, the AAV serotype is a serotype comprising the G2B5 peptide or a variant thereof.

In certain embodiments, the AAV serotype is VOY101 or a variant thereof In certain embodiments, the VOY101 comprises the amino acid sequence of SEQ ID NO: 1. In certain embodiments, the capsid sequence comprises the nucleic acid sequence of SEQ ID NO:1722.

In certain embodiments, the AAV serotype is VOY201 or a variant thereof In certain embodiments, the VOY201 comprises the amino acid sequence of SEQ NO: 1724. In certain embodiments, the capsid sequence comprises the nucleic acid sequence of SEQ ID NO: 1723.

In certain embodiments, the AAV capsid allows for blood brain harrier penetration following intravenous administration. Non-limiting examples of such AAV capsids comprise AAV9, AAV9 K449R, VOY101, VOY201, or AAV capsids comprising a peptide insert such as, but not limited to, AAVPHP.N (PHRN), AAVPHP.B (PHP.B), PHP.S, G2A3, G2B4, G2B5, G2A12, G2A15, PHP.B2, PHP.B3, or AAVPHP.A (PHP.A),

In certain embodiments, the AAV serotype may comprise a capsid amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above. In certain embodiments, the AAV serotype comprises a capsid amino acid sequence at least 80% identical to SEQ ID NO: 1, 2, 3, 9, 136, or 1724. In certain embodiments, the AAV serotype comprises a capsid amino acid sequence at least 85% identical to SEQ ID NO: 1, 2, 3, 9, 136, or 1724. In certain embodiments, the AAV serotype comprises a capsid amino acid sequence at least 90% identical to SEQ ID NO: 1, 2, 3, 9, 136, or 1724. In certain embodiments, the AAV serotype comprises a capsid amino acid sequence at least 95% identical to SEQ ID NO: 1, 2, 3, 9, 136, or 1724. In certain embodiments, the AAV serotype comprises a capsid amino acid sequence at least 99% identical to SEQ ID NO: 1, 2, 3, 9, 136, or 1724. In certain embodiments, the AAV serotype comprises a capsid amino acid of SEQ ID NO: 1, 2, 3, 9, 136, or 1724.

In certain embodiments, the AAV serotype may be encoded by a capsid nucleic acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%. 78%, 79%. 80%, 81%, 82%. 83%, 84%, 85%. 86%, 87%, 88%. 89%, 90%, 91%. 92%, 93%, 94%, 95%, 96%, 97%, 98%, 99%, or 100% identity to any of the those described above. In certain embodiments, the AAV serotype comprises a capsid nucleic acid sequence at least 80% identical to SEQ ID NO: 4, 135, 1722. or 1723. In certain embodiments, the AAV serotype comprises a capsid nucleic acid sequence at least 85% identical to SEQ ID NO: 4, 135, 1722, or 1723. In certain embodiments, the AAV serotype comprises a capsid nucleic acid sequence at least 90% identical to SEQ ID NO: 4, 135, 1722, or 1723. In certain embodiments, the AAV serotype comprises a capsid nucleic acid sequence at least 95% identical to SEQ ID NO: 4, 135, 1722, or 1723. In certain embodiments, the AAV serotype comprises a capsid nucleic acid sequence at least 99% identical to SEQ ID NO: 4, 135, 1722, or 1723. In certain embodiments, the AAV serotype comprises a capsid nucleic acid sequence of SEQ ID NO: 4, 135. 1722, or 1723.

In certain embodiments, the initiation codon for translation of the AAV VP1 capsid protein may be CTG, TTG, or GTG as described in U.S. Pat. No. 8,163,543 (the content of which is incorporated herein by reference in its entirety as related to AAV capsids and start codons, insofar as it does not conflict with the present disclosure).

The present disclosure refers to structural capsid proteins (comprising VP1, VP2, and. VP3), which are encoded by capsid (Cap) genes. These capsid proteins form an outer protein structural shell (i.e. capsid) of a viral vector such as AAV. VP capsid proteins synthesized from Cap polynucleotides generally comprise a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two, or three) VP capsid proteins comprising the viral capsid may be produced, some of which may comprise a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are incorporated herein by reference in their entireties as related to Met/AA-clipping in capsid proteins, insofar as they do not conflict with the present disclosure.

According to the present disclosure, references to capsid proteins are not limited to either clipped (Met−/AA−) or unclipped (Met+/AA+) sequences and may, in context, refer to independent capsid proteins, viral capsids comprised of a mixture of capsid proteins, and/or polynucleotide sequences (or fragments thereof) which encode, describe, produce, or result in capsid proteins of the present disclosure. A direct reference to a “capsid protein” or “capsid polypeptide” (such as VP1, VP2, or VP3) may also comprise VP capsid proteins which comprise a Met1/AA1 amino acid (Met+/AA+) as well as corresponding VP capsid proteins which lack the Met1/AA1amino acid as a result of Met/AA-clipping (Met−/AA−).

Further, according to the present disclosure, a reference to a specific SEQ ID NO (whether a protein or nucleic acid) that comprises or encodes, respectively, one or more capsid proteins that comprise a Met1/AA1amino acid (Met+/AA+) should be understood to teach the VP capsid proteins that lack the Met1/AA1 amino acid as upon review of the sequence, it is readily apparent any sequence that merely lacks the first listed amino acid (whether or not methionine).

As a non-limiting example, reference to a VP1 polypeptide sequence which is 736 amino acids in length and which comprises a “Met1” amino acid (Met+) encoded by the AUG/ATG start codon may also be understood to teach a VP1 polypeptide sequence that is 735 amino acids in length and that does not comprise the “Met1” amino acid (Met−) of the 736 amino acid Met+ sequence. As a second non-limiting example, reference to a VP1 polypeptide sequence that is 736 amino acids in length and that comprises an “AA1” amino acid (AA1+) encoded by any NNN initiator codon may also be understood to teach a VP1 polypeptide sequence that is 735 amino acids in length and that does not comprise the “AA1” amino acid (AA1−) of the 736 amino acid AA1+ sequence.

References to viral capsids foamed. from VP capsid proteins (such as reference to specific AAV capsid serotypes) can incorporate VP capsid proteins that comprise a Met1/AA1 amino acid (Met+/AA1+), corresponding VP capsid proteins that lack the Met1/AA1 amino acid as a result of Met/AA1-clipping (Met−/AA1−), or combinations thereof (Met+/AA1+ and Met−/AA1−).

As a non-limiting example, an AAV capsid serotype can comprise VP1 (Met+/AA1+), VP1 (Met−/AA1−), or a combination of VP1 (Met+/AA1+) and VP1 (Met-/AA1−). An AAV capsid serotype can also comprise VP3 (Met+/AA1+), VP3 (Met−/AA1−), or a combination of VP3 (Met+/AA1+) and VP3 (Met−/AA1−); and can also comprise similar optional combinations of VP2 (Met+/AA1) and VP2 (Met−/AA1−).

### Payloads

AAV particles of the present disclosure can comprise, or be produced using, at least one payload construct which comprises at least one payload region. In certain embodiments, the payload region may be located within a viral genome, such as the viral genome of a payload construct, At the 5′ and/or the 3′ end of the payload region there may be at least one inverted terminal repeat (ITR). Within the payload region, there may be a promoter region, an intron region and a coding region.

In certain embodiments, a payload construct of the present disclosure can be a bacmid, also known as a baculovims plasmid or recombinant baculovinis genome.

In certain embodiments, the payload region of the AAV particle comprises one or more nucleic acid sequences encoding a polypeptide or protein of interest.

In certain embodiments, the AAV particle comprises a viral genome with a payload region comprising nucleic acid sequences encoding more than one polypeptide of interest. In certain embodiments, a viral genome encoding one or more polypeptides may be replicated and packaged into a viral particle. A target cell transduced with a viral particle comprising the vector genome may express each of the one or more polypeptides in the single target cell.

Where the AAV particle payload region encodes a polypeptide, the polypeptide may be a peptide, polypeptide or protein. As a non-limiting example, the payload region may encode at least one therapeutic protein of interest. The AAV viral genomes encoding polypeptides described herein may be useful in the fields of human disease, viruses, infections veterinary applications and a variety of in vivo and in vitro settings.

In certain embodiments, administration of the formulated AAV particles (which comprise the viral genome) to a subject will increase the expression of a protein in a subject, In certain embodiments, the increase of the expression of the protein will reduce the effects and/or symptoms of a disease or ailment associated with the polypeptide encoded by the payload.

In certain embodiments, the AAV particle comprises a viral genome with a payload region comprising a nucleic acid sequence encoding a protein of interest (i.e. a payload protein, therapeutic protein)

In certain embodiments, the payload region comprises a nucleic acid sequence encoding a protein comprising but not limited to an antibody, Aromatic L-Amino Acid Decarboxylase (AADC). ApoE2, Frataxin, survival motor neuron (SAM) protein, glucocerebrosidase, N-sulfogiucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLNS, CLN6 (linclin), NIFSD8, CLN8, aspartoacylase (ASPA), progranulin (GRN). MeCP2, beta-galactosidase (GLB1) and/or gigaxonin (GAN).

In certain embodiments, the AAV particle comprises a viral genome with a payload region comprising a nucleic acid sequence encoding any of the disease-associated proteins (and fragment or variants thereof) described in any one of the following international Publications: WO2016073693, WO2017023724, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each incorporated herein by reference in their entireties insofar as they do not conflict with the present disclosure.

Amino acid sequences encoded by payload regions of the viral genomes of the disclosure may be translated as a whole polypeptide, a plurality of polypeptides or fragments of polypeptides, which independently may be encoded by one or more nucleic acids, fragments of nucleic acids or variants of any of the aforementioned, As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides comprise gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

In certain embodiments a “polypeptide variant” is provided. The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and in certain embodiments, they will be at least about 80%, or at least about 90% identical (homologous) to a native or reference sequence.

The present disclosure comprises the use of formulated AAV particles whose vector genomes encode modulatory polynucleotides, e.g., RNA or DNA molecules as therapeutic agents. Accordingly, the present disclosure provides vector genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the AAV particle comprises a viral genome with a payload region comprising a nucleic acid sequence encoding or comprising one or more modulatory polynucleotides. In certain embodiments, the AAV particle comprises a viral genome with a payload region comprising a nucleic acid sequence encoding a modulatory polynucleotide of interest. In certain embodiments of the present disclosure, modulatory polynucleotides, e.g, RNA or DNA molecules, are presented as therapeutic agents. RNA interference mediated gene silencing can specifically inhibit targeted gene expression.

In certain embodiments, the payload region comprises a nucleic acid sequence encoding a modulatory polynucleotide which interferes with a target gene expression and/or a target protein production. In certain embodiments, the gene expression or protein production to be inhibited/modified may comprise but are not limited to superoxide dismutase 1 (SOD1), chromosome 9 open reading frame 72 (C9ORF72), TAR DNA binding protein (TARDBP), ataxin-3 (ATXN3), huntingtin amyloid precursor protein (APP), apolipoprotein E (ApoE), microtubule-associated protein tau (MAPT), alpha-synuclein (SNCA), voltage-gated sodium channel alpha subunit 9 (SCN9A), and/or voltage-gated sodium channel alpha subunit 10 (SCN10A).

In certain embodiments, the AAV particle comprises a viral genome with a payload region comprising a nucleic acid sequence encoding any of the modulatory polynucleotides, RNAi molecules, siRNA molecules, dsRNA molecules, and/or RNA duplexes described in any one of the following International Publications: WO2016073693, WO2017023724, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each incorporated herein by reference in their entireties insofar as they do not conflict with the present disclosure.

In certain embodiments, a nucleic acid sequence encoding such siRNA. molecules, or a single strand of the siRNA molecules, is inserted into adeno-associated viral vectors and introduced into cells, specifically cells in the central nervous system.

AAV particles have been investigated for siRNA delivery because of several unique features. Non-limiting examples of the features comprise (i) the ability to infect both dividing and non-dividing cells; (ii) a broad host range for infectivity, comprising human cells; (iii) wild-type AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

In certain embodiments, the encoded siRNA duplex of the present disclosure contains an antisense strand and a sense strand hybridized together forming a duplex structure, wherein the antisense strand is complementary to the nucleic acid sequence of the targeted gene of interest, and wherein the sense strand is homologous to the nucleic acid sequence of the targeted gene of interest. In other aspects, there are 0, 1 or 2 nucleotide overhangs at the 3′end of each strand.

The payloads of the formulated AAV particles of the present disclosure may encode one or more agents which are subject to RNA interference (RNAi) induced inhibition of gene expression. Provided herein are encoded siRNA duplexes or encoded dsRNA that target a gene of interest (referred to herein collectively as “siRNA molecules”). Such siRNA molecules, e.g., encoded siRNA duplexes, encoded dsRNA or encoded siRNA or dsRNA precursors can reduce or silence gene expression in cells, for example, astrocytes or microglia, cortical, hippocampal, entorhinal, thalamic, sensory or motor neurons.

RNAi (also known as post-transcriptional gene silencing (PIGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2-nucleotide 3′ overhangs and that match the nucleic acid sequence of the target gene. These short RNA species may be naturally produced in vivo by Dicer-mediated cleavage of larger dsRNAs and they are functional in mammalian cells.

Naturally expressed small RNA molecules, known as microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA mediated down regulation of gene expression may he caused by cleavage of the target mRNAs, translational inhibition of the target mRNAs, or mRNA decay. miRNA targeting sequences are usually located in the 3′ UTR of the target mRNAs. A single miRNA may target more than 100 transcripts from various genes, and one mRNA may be targeted by different miRNAs.

siRNA duplexes or dsRNA targeting a specific mRNA may be designed as a payload of an AAV particle and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498), Since this initial report, post-transcriptional gene silencing by siRNAs quickly emerged as a powerful tool for genetic analysis in mammalian cells and has the potential to produce novel therapeutics.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases it requires higher concentration of the ss-siRNA to achieve the effective gene silencing potency of the corresponding duplex.

In certain embodiments, the siRNA molecules may be encoded in a modulatory polynucleotide which also comprises a molecular scaffold. As used herein a “molecular scaffold” is a framework or starting molecule that forms the sequence or structural basis against which to design or make a subsequent molecule.

In certain embodiments, the modulatory polynucleotide which comprises the payload (e.g., siRNA, miRNA or other RNAi agent described herein) comprises molecular scaffold which comprises a leading 5′ flanking sequence which may be of any length and may be derived in whole or in part from wild type microRNA sequence or be completely artificial. A 3′ flanking sequence may mirror the 5′ flanking sequence in size and origin. In certain embodiments, one or both of the 5′ and 3′ flanking sequences are absent.

In certain embodiments, the molecular scaffold may comprise one or more linkers known in the art. The linkers may separate regions or one molecular scaffold from another. As a non-limiting example, the molecular scaffold may be polycistronic.

In certain embodiments, the modulatory polynucleotide is designed using at least one of the following properties: loop variant, seed mismatch/bulge/wobble variant, stern mismatch, loop variant and basal stem mismatch variant, seed mismatch and basal stem mismatch variant, stem mismatch and basal stem mismatch variant, seed wobble and basal stem wobble variant, or a stem sequence variant.

### Payloads: Polypeptides and Variants

In certain embodiments, the payload region of the AAV particle comprises one or more nucleic acid sequences encoding a polypeptide or protein of interest.

In certain embodiments, the AAV particle comprises a viral genome with a payload region comprising nucleic acid sequences encoding more than one polypeptide of interest. In certain embodiments, a viral genome encoding one or more polypeptides may be replicated and packaged into a viral particle. A target cell transduced with a viral particle comprising the vector genome may express each of the one or more polypeptides in the single target cell.

Where the AAV particle payload region encodes a polypeptide, the polypeptide may be a peptide, polypeptide or protein. As a non-limiting example, the payload region may encode at least one therapeutic protein of interest. The AAV viral genomes encoding polypeptides described herein may be useful in the fields of human disease, viruses, infections veterinary applications and a variety of in vivo and in vitro settings.

In certain embodiments, administration of the formulated AAV particles (which comprise the viral genome) to a subject will increase the expression of a protein in a subject. In certain embodiments, the increase of the expression of the protein will reduce the effects and/or symptoms of a disease or ailment associated with the polypeptide encoded by the payload.

In certain embodiments, the formulated AAV particles of the present disclosure may be used to reduce the decline of functional capacity and activities of daily living as measured by a standard evaluation system such as, but not limited to, the total functional capacity (TFC) scale.

In certain embodiments, the AAV particle comprises a viral genome with a payload region comprising a nucleic acid sequence encoding a protein of interest (i.e. a payload protein, therapeutic protein).

In certain embodiments, the payload region comprises a nucleic acid sequence encoding a protein comprising but not limited to an antibody, Aromatic L-Amino Acid Decarboxylase (AADC), ApoE2, Frataxin, survival motor neuron (SMN) protein, glucocerebrosidase, N-sulfogiucosamine sulfohydmlase, N-acetyl-alpha-alucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspanoacylase (ASPA), progranulin (GRN), MeCP2, beta-galactosidase (GLB 1) and/or gigaxonin (GAN).

In certain embodiments, the AAV particle comprises a viral genome with a payload region comprising a nucleic acid sequence encoding AADC or any other payload known in the art for treating Parkinson's disease, As a non-limiting example, the payload may comprise a sequence such as NM_001082971.1 (GI: 132814447), NM_000790.3 (GI: 132814459), NM_001242886.1 (GI: 338968913), NM_001242887.1 (GI: 338968916), NM_001242888.1 (GI: 338968918), NM_001242889.1 (GI: 338968920), NM_001242890.1 (GI: 338968922) and fragment or variants thereof

In certain embodiments, the AAV particle comprises a viral genome with a payload region comprising a nucleic acid sequence encoding frataxin or any other payload known in the art for treating Friedreich's Ataxia. As a non-limiting example, the payload may comprise a sequence such as NM_000144.4 (GI: 239787167), NM_181425. (GI: 239787185), NM_001161706.1 (GI: 239787197) and fragment or variants thereof.

In certain embodiments, the AAV particle comprises a viral genome with a payload region comprising a nucleic acid sequence encoding SMN or any other payload known in the art for treating spinal muscular atrophy (SMA). As a non-limiting example, the payload may comprise a sequence such as NM_001297715.1 (GI: 663070993), NM_000344.3 (GI: 196115055), NM_022874.2 (GI: 196115040) and fragment or variants thereof.

In certain embodiments, the AAV particle comprises a viral genome with a payload region comprising a nucleic acid sequence encoding any of the disease-associated proteins (and fragment or variants thereof) described in U.S. Patent publication No. 20180258424; the content of which is herein incorporated by reference in its entirety.

In certain embodiments, the AAV particle comprises a viral genome with a payload region comprising a nucleic acid sequence encoding any of the disease-associated proteins (and fragment or variants thereof) described in any one of the following International Publications: WO2016073693, WO2017023724, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety.

In certain embodiments, the formulated AAV particles of the present disclosure may be used to improve performance on any assessment used to measure symptoms of a neurodegenerative disorder/disease. Such assessments comprise, but are not limited to ADAS-cog (Alzheimer Disease Assessment Scale—cognitive), NIMSE (Mini-Mental State Examination), GDS (Geriatric Depression Scale). FAQ (Functional Activities Questionnaire), AM, (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure. Instrumental Activities of Daily Living, IQCODE (Informant Questionnaire on Cognitive Decline in the Elderly). Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B Ther Adv Neurol Disord 5(6):349-358 (2012), the contents of which are herein incorporated by reference in their entirety.

In certain embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

In certain embodiments an “amino acid sequence variant” is provided. The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not he confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least about 70% homology to a native sequence, and in certain embodiments, they will be at least about 80% or at least about 90% homologous to a native sequence. “Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximwn percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to comprise polypeptide variants which differ by one or more amino acid alterations, e.g., substitutions, additions or deletions of amino acid residues that still maintain the properties of the parent polypeptide.

Sequence tags or amino acids, such as one or more lysines, can be added to the peptide sequences of the disclosure (e.g., at the N-terminal or C-terminal ends). Sequence tags can he used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively he deleted depending on the use of the sequence, as for example, expression of the sequence as part of a larger sequence which is soluble or linked to a solid support.

In certain embodiments a “substitutional variant” is provided. “Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions comprise the substitution of a non-polar (hydrophobic) residue such as isoleucine, valine and leucine for another non-polar residue. Likewise, examples of conservative substitutions comprise the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions comprise the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In certain embodiments an “insertional variant” is provided. “Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

In certain embodiments a “deletional variant” is provided. “Deletional variants” when referring to proteins, are those with one or more amino acids in the native or starting amino acid sequence removed. Ordinarily, deletional variants will have one or more amino acids deleted in a particular region of the molecule.

As used herein, the term “derivative” is used synonymously with the term “variant” and refers to a molecule that has been modified or changed in any way relative to a reference molecule or starting molecule. In certain embodiments, derivatives comprise native or starting proteins that have been modified with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartylresidues. Alternatively, these residues are deamidated under mildly acidic conditions. Either form of these residues may be present in the proteins used in accordance with the present disclosure.

Other post-translational modifications comprise hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

“Features” when referring to proteins are defined as distinct amino acid sequence-based components of a molecule. Features of the proteins of the present disclosure comprise surface manifestations, local conformational shape, folds, loops, half-loops, domains, half-domains, sites, termini or any combination thereof.

As used herein when referring to proteins the term “surface manifestation” refers to a polypeptide-based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds comprise beta sheets and alpha helices. Examples of tertiary folds comprise domains and regions formed due to aggregation or separation of energetic forces. Regions formed in this way comprise hydrophobic and hydrophilic pockets, and the like.

As used herein the terns “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid residues as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+1−0.5 being 3 or 4).

As used herein when referring to proteins the term “domain” refers to a motif of a polypeptide having one or more identifiable structural or functional characteristics or properties (e.g., binding capacity, serving as a site for protein-protein interactions).

As used herein when referring to proteins the term “half-domain” means portion of an identified domain having at least half the number of amino acid residues as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid -based embodiments is used synonymous with “amino acid residue” and “amino acid side chain”. A site represents a position within a peptide or polypeptide that may be modified, manipulated, altered, derivatized or varied within the polypeptide-based molecules of the present disclosure.

As used herein the terms “termini or terminus” when referring to proteins refers to an extremity of a peptide or polypeptide. Such extremity is not limited only to the first or final site of the peptide or polypeptide but may comprise additional amino acids in the terminal regions. The polypeptide-based molecules of the present disclosure may be characterized as having both an N-terminus (terminated by an amino acid with a free amino group (NH2)) and a C-terminus (terminated by an amino acid with a free carboxyl group (COOH)). Proteins of the disclosure are in certain embodiments made up of multiple polypeptide chains brought together by disulfide bonds or by non-covalent forces (multimers. oligomers). These sorts of proteins will have multiple N- and C-termini. Alternatively, the termini of the polypeptides may he modified such that they begin or end, as the case may be, with a non-polypeptide-based moiety such as an organic conjugate.

Once any of the features have been identified or defined as a component of a molecule of the disclosure, any of several manipulations and/or modifications of these features may be performed by moving, swapping, inverting, deleting, randomizing or duplicating. Furthermore, it is understood that manipulation of features may result in the same outcome as a modification to the molecules of the disclosure. For example, a manipulation which involves deleting a domain would result in the alteration of the length of a molecule just as modification of a nucleic acid to encode less than a full-length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

### Payloads: Modulatoty Polynucleotides Targeting a Gene of Interest

### General

In certain embodiments, the present disclosure presents the use of formulated AAV particles whose vector genomes encode modulatory polynucleotides, e.g., RNA or DNA molecules as therapeutic agents. Accordingly, the present disclosure provides vector genomes which encode polynucleotides which are processed into small double stranded RNA (dsRNA) molecules (small interfering RNA, siRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the AAV particle comprises a viral genome with a payload region comprising a nucleic acid sequence encoding or comprising one or more modulatory polynucleotides. In certain embodiments, the AAV particle comprises a viral genome with a payload region comprising a nucleic acid sequence encoding a modulatory polynucleotide of interest. In certain embodiments of the present disclosure, modulatory polynucleotides, e.g., RNA or DNA molecules, are presented as therapeutic agents. RNA interference mediated gene silencing can specifically inhibit targeted gene expression.

In certain embodiments, the payload region comprises a nucleic acid sequence encoding a modulatory polynucleotide which interferes with a target gene expression and/or a target protein production. In certain embodiments, the gene expression or protein production to be inhibited/modified may comprise but are not limited to superoxide dismutase 1 (SOD1), chromosome 9 open reading frame 72 (C9OR1⁷72), TAR DNA binding protein (TARDBP), ataxin-3 (ATXN3), huntingtin (FITT), amyloid precursor protein (APP), apolipoprotein E (ApoE), microtubule-associated protein tau (MAPT), alpha-synuclein (SNCA.), voltage-gated sodium channel alpha subunit 9 (SCN9A), and/or voltage-gated sodium channel alpha subunit 10 (SCN10A).

The present disclosure provides small interfering RNA (siRNA) duplexes (and modulatory polynucleotides encoding them) that target SOD1 mRNA to interfere with the gene expression and/or protein production of SOD1. The present disclosure also provides methods of their use for inhibiting gene expression and protein production of an allele of SOD1, for treating amyotrophic lateral sclerosis (ALS). In certain embodiments, the siRNA duplexes of the present disclosure may target SOD1 along any segment of the respective nucleotide sequence. In certain embodiments, the siRNA duplexes of the present disclosure may target SOD1 at the location of a SNP or variant within the nucleotide sequence.

The present disclosure provides small interfering RNA (siRNA) duplexes (and modulatory polynucleotides encoding them) that target HTT mRNA to interfere with the gene expression and/or protein production of HIT. The present disclosure also provides methods of their use for inhibiting gene expression and protein production of an allele of HTT, for treating Huntington's disease (HD). In certain embodiments, the siRNA duplexes of the present disclosure may target HTT along any segment of the respective nucleotide sequence. In certain embodiments, the siRNA duplexes of the present disclosure may target HIT at the location of a SNP or variant within the nucleotide sequence,

In certain embodiments, the AAV particle comprises a viral genome with a payload region comprising a nucleic acid sequence encoding any of the modulatory polynucleotides, RNAi molecules, siRNA molecules, dsRNA molecules, and/or RNA duplexes described in any one of the following international Publications: WO2016073693, WO2017023724, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety.

In certain embodiments, a nucleic acid sequence encoding such siRNA molecules, or a single strand of the siRNA molecules, is inserted into adeno-associated viral vectors and introduced into cells, specifically cells in the central nervous system.

AAV particles have been investigated for siRNA delivery because of several unique features. Non-limiting examples of the features comprise (i) the ability to infect both dividing and non-dividing cells; (ii) a broad host range for infectivity, comprising human cells; (iii) AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

In certain embodiments, the encoded siRNA duplex of the present disclosure contains an antisense strand and a sense strand hybridized together forming a duplex structure, wherein the antisense strand is complementary to the nucleic acid sequence of the targeted gene of interest, and wherein the sense strand is homologous to the nucleic acid sequence of the targeted gene of interest. In other aspects, there are 0, 1 or 2 nucleotide overhangs at the 3′ end of each strand.

According to the present disclosure, each strand of the siRNA duplex targeting the gene of interest can be about 19 to 25, 19 to 24 or 19 to 21 nucleotides in length, such as about 19 nucleotides, 20 nucleotides, 21 nucleotides, 22 nucleotides, 23 nucleotides, 24 nucleotides, or 25 nucleotides in length.

In certain embodiments, an siRNA or dsRNA comprises at least two sequences that are complementary to each other. The dsRNA comprises a sense strand having a first sequence and an antisense strand having a second sequence. The antisense strand comprises a nucleotide sequence that is substantially complementary to at least part of an mRNA encoding a gene of interest, and the region of complementarity is 30 nucleotides or less, and at least 15 nucleotides in length. Generally, the dsRNA is 19 to 25, 19 to 24 or 19 to 21 nucleotides in length. In certain embodiments, the dsRNA is from about 15 to about 25 nucleotides in length, and in certain embodiments the dsRNA is from about 25 to about 30 nucleotides in length.

The dsRNA encoded in an expression vector upon contacting with a cell expressing protein encoded by the gene of interest, inhibits the expression of protein encoded by the gene of interest by at least 10%, at least 20%, at least 25%, at least 30%, at least 35%, at least 40%, at least 50%, at least 60%, at least 70%, at least 80%, or more, when assayed by methods known in the art or a method as described herein.

According to the present disclosure, formulated AAV particles comprising the nucleic acids of the siRNA duplexes, one strand of the siRNA duplex or the dsRNA targeting the gene of interest are produced, the AAV particle serotypes may be PHP.B, PHPA, G2B-26, G2B-13, TH1.1-32, TH1.1-35, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42,12, AAV42-1b, AAV42-2, AAV42-3a, AAV42-3b, AAV42-4, AAV42-5a, AAV42-5b, AAV42-6b, AAV42-8, AAV42, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223,2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh,57, AAV5-22/rh.58, AAV7.3/hu.7, AAV16.8/hu.10, AAV16.12/hu.11, AAV29.3/bb.1, AAV29,5/bb.2, AAV106.1/hu.37, AAV114.3/hu.40, AM/127.2/1m.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV1.61.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh,55, AAVrh.47, AAVrh.69, AAVrh.45, AAVrh.59, AAVhu.12, AAVH16, AAVLK03, AAVH-1/hu.1, AAVH5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy6, AAVhu.1, AAVhu.2, AAVhu.3, AAVhu.4, AAVhu.5. AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu,23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.54, AAVhu.55, AAVhu.56, AAVhu.57, AAVhu.58, AAVhu.60, AAVhu.61, AAVhu.63, AAVhu.64-AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, ovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T , AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK16, AAV-LK AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101 , AAV-8h, AAV-8b, AAV-h, AAV-b, AAV SM 10-2, AAV Shuffle 100-1 , AAV Shuffle 100-3, AAV Shuffle 100-7-NAV Shuffle 10-2. AAV Shuffle 10-6, AAV Shuffle 10-8. AAV Shuffle 100-2, AAV SM 10-1. AAV SM 10-8 , AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes. AAV CBr-7.1, AAV CBr-7.10, AAV C9r-7.2,NAV CBr-7.3, AAV CBr-7.4, AAV CBr-7.5, AAV CBr-7.7, AAV CBr-7.8 AAV CBr-B7.3, AAV CBr-B7.4, AAV CBr-E1l, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd.-136, AAV CKd.-B7,NAV CKd.-138, AAV CKd-H1, CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV AAV CLg-F3, AAV CLg-F4, AAV CLg-F5, AAV CLg-F6, AAV CLg-F7, AAV CLg-F8, AAV AAV CLv1-1, AAV Clv1-10, AAV CLv1-12, AAV CLv1-3,AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AA CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/1-HSC6, AAVF7/HSC7, AAVF8/HSC8, and/or AAVF9/HSC9 and variants thereof.

According to the present disclosure, the siRNA molecules arc designed and tested for their ability in reducing inRNA levels in cultured cells.

In certain embodiments, the siRNA molecules are designed and tested for their ability in reducing levels of the gene of interest in cultured cells.

The present disclosure also provides pharmaceutical compositions comprising at least one siRNA duplex targeting the gene of interest and a pharmaceutically acceptable carrier. In some aspects, the siRNA duplex is encoded by a vector genome in an AAV particle.

In certain embodiments, the present disclosure provides methods for inhibiting/silencing gene expression in a cell. In some aspects, the inhibition of gene expression refers to an inhibition by at least about 20%, such as by at least about 30%, 40%. 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 35-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%. 90-95%, 90-100% or 95-100%. Accordingly, the protein product of the targeted gene may be inhibited by at least about 20%, such as by at least about 30%, 40%, 50©′0 60%, 70%. 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In certain embodiments, the encoded siRNA duplexes may be used to reduce the expression of protein encoded by the gene of interest by at least about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 35-40%, 30-50%, 30-60%, 30-70%, 30-80%, 30-90%, 30-95%, 30-100 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%. 60-95%, 60-100%, 70-80%, 70-90%, 70-95%, 70-100%, 80-90%, 80-95%, 80-100%, 90-95%, 90-100% or 95-100%. As a non-limiting example, the expression of protein may be reduced 50-90%. As a non-limiting example, the expression of protein may be reduced 30-70%. As a non-limiting example, the expression of protein may he reduced 40-70%.

In certain embodiments, the encoded siRNA duplexes may be used to reduce the expression of mRNA transcribed from the gene of interest by at least about 20%, 30%, 40%, 50%, 60%, 70%, 80%, 85%, 90%, 95% and 100%, or at least 20-30%, 20-40%, 20-50%. 20-60%, 20-70%, 20-80%, 20-90%, 20-95%, 20-100%, 30-40%, 35-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of mRNA expression may be reduced 50-90%.

In certain embodiments, the encoded siRNA duplexes may be used to reduce the expression of protein encoded by the gene of interest and/or transcribed mRNA in at least one region of the CNS. The expression of protein and/or mRNA is reduced by at least about 20%, 30%, 40%, 50%, 60%, 70%, 80%, 85%. 90%, 95% and 100%, or at least 20-30%, 20-40%, 20-50%, 20-60%, 20-70%, 20-80%, 20-90%, 20-95%, 20-100%, 30-40%, 35-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%. 90-100% or 95-100% in at least one region of the CNS. As a non-limiting example, the region is the neurons (e.g., cortical neurons).

In certain embodiments, the formulated AAV particles comprising such encoded siRNA molecules may be introduced directly into the central nervous system of the subject, for example, by infusion into the putamen,

In certain embodiments, the formulated AAV particles comprising such encoded siRNA molecules may be introduced directly into the central nervous system of the subject, for example, by infusion into the thalamus of a subject.

In certain embodiments, the formulated AAV particles comprising such encoded siRNA molecules may be introduced directly into the 1 nervous system of the subject, for example, by infusion into the white matter of a subject.

In certain embodiments, the formulated AAV particles comprising such encoded siRNA molecules may be introduced to the central nervous system of the subject, for example, by intravenous administration to a subject.

In certain embodiments, the pharmaceutical composition of the present disclosure is used as a solo therapy. In certain embodiments, the pharmaceutical composition of the present disclosure is used in combination therapy. The combination therapy may be in combination with one or more neuroprotective agents such as small molecule compounds, growth factors and hormones which have been tested for their neuroprotective effect on motor neuron degeneration.

siRNA Molecules

The payloads of the formulated AAV particles of the present disclosure may encode one or more agents which are subject to RNA interference (RNAi) induced inhibition of gene expression. Provided herein are encoded siRNA duplexes or encoded dsRNA that target a gene of interest (referred to herein collectively as “siRNA molecules”). Such siRNA molecules, e.g., encoded siRNA duplexes, encoded dsRNA or encoded siRNA or dsRNA precursors can reduce or silence gene expression in cells, for example, astrocytes or microglia, cortical, hippocampal, entorhinal, thalamic, sensory or motor neurons.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2-nucleotide 3′ overhangs and. that match the nucleic acid sequence of the target gene. These short RNA species may be naturally produced in vivo by Dicer-mediated cleavage of larger dsRNAs and they are functional in mammalian cells.

Naturally expressed small. RNA molecules, known as microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region, miRNA mediated down regulation of gene expression may be caused by cleavage of the target mRNAs, translational inhibition of the target mRNAs, or mRNA decay. miRNA targeting sequences are usually located in the 3′ UTR of the target mRNAs. A single miRNA may target more than 100 transcripts from various genes, and one mRNA may be targeted by different miRNAs.

siRNA duplexes or dsRNA targeting a specific mRNA may be designed as a payload of an AAV particle and introduced into cells for activating RNAi processes. Elbashir et al, demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful. tool for genetic analysis in mammalian cells and has the potential to produce novel therapeutics.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g. antisense strand RNA or antisense oligonucleotides). In many cases it requires higher concentration of the ss-siRNA to achieve the effective gene silencing potency of the corresponding duplex.

Any of the foregoing molecules may be encoded by an AAV particle or vector genome.

Design and Sequences of siRNA Duplexes Targeting a Gene of Interest

Some guidelines for designing siRNAs, e.g., herein encoded as a payload in a vector genome, have been proposed in the art. These guidelines generally recommend generating a 19-nucleotide duplexed region, symmetric 2-3 nucleotide 3′overhangs, 5-phosphate and 3-hydroxyl groups targeting a region in the gene to be silenced. Other rules that may govern siRNA sequence preference comprise, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such consideration, together with the specific sequence of a target gene, highly effective siRNA molecules essential for suppressing mammalian target gene expression may be readily designed.

In certain embodiments, an siRNA molecule of the present disclosure comprises a sense strand and a complementary antisense strand in which both strands are hybridized together to form a duplex structure. The antisense strand has sufficient complementarity to the mRNA sequence to direct target-specific RNAi, i.e., the siRNA. molecule has a sequence sufficient to trigger the destruction of the target mRNA by the RNAi machinery or process.

In certain embodiments, the antisense strand and target mRNA sequences have 100% complementarity. The antisense strand may be complementary to any part of the target mRNA sequence.

In certain embodiments, the antisense strand and target mRNA sequences comprise at least one mismatch. As a non-limiting example, the antisense strand and the target mRNA sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

According to the present disclosure, encoded the siRNA molecule has a length from about 10-50 or more nucleotides, i.e., each strand comprising 10-50 nucleotides (or nucleotide analogs). In certain embodiments, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementary to a target region. In certain embodiments, the encoded siRNA molecule has a length from about 19 to 25. 19 to 24 or 19 to 21 nucleotides.

In certain embodiments, the encoded siRNA molecules of the present disclosure may comprise a region of or encoding the nucleotide sequence of a gene of interest (e.g., sense or passenger sequence). As a non-limiting example, the sense sequence used in the siRNA molecule of the present disclosure has an identity which is at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98% or 99% or at least 20-30%. 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% to a portion of the nucleotide sequence of the gene of interest or encoding the gene of interest. As another non-limiting example, the sense sequence used in the siRNA molecule of the present disclosure comprises at least 3, 4, 5, 6, 7, 8, 9, 10, 11, 12, 13, 14, 15, 16, 17, 18, 19, 20, 21 or more than 21 consecutive nucleotides of the nucleotide sequence of the gene of interest or encoding the gene of interest.

In certain embodiments, the encoded siRNA molecules of the present disclosure may comprise a region of a nucleotide sequence of the gene of interest or encoding the gene of interest (e.g., antisense or guide sequence) such as, but not limited to, at least 3, 4, 5, 6, 7, 8, 9, 10, 11, 12, 13, 14, 15, 16, 17, 18, 19, 20, 21 or more than 21 nucleotides which are the reverse complement the nucleotide sequence of the gene of interest or fragment or variant thereof. As a non-limiting example, the antisense sequence used in the encoded siRNA molecule of the present disclosure has a reverse complement which is at least 30%, 40%, 50%, 60%, 70%, 80%. 81%, 82%, 83%. 84%, 85%, 86%. 87%, 88%, 89%. 90%, 91%, 92%. 93%, 94%, 95%, 96%, 97%, 98% or 99% or at least 20-30%. 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% to a portion of a nucleotide sequence of the gene of interest or encoding the gene of interest. As another non-limiting example, the antisense sequence used in the siRNA molecule of the present disclosure comprises at least 3, 4, 5, 6, 7, 8, 9, 10, 11, 12, 13, 14, 15, 16, 17, 18, 19, 20, 21 or more than 21 consecutive nucleotides which are the reverse complement of a nucleotide sequence of the gene of interest or encoding the gene of interest.

In certain embodiments, the encoded siRNA molecules of the present disclosure may comprise an antisense sequence and a sense sequence, or a fragment or variant thereof. As a non-limiting example, the antisense sequence and the sense sequence have at least 30%, 40%, 50%, 60%, 70%, 80%, 81%, 82%, 83%, 84%, 85%, 86%, 87%, 88%, 89%, 90%, 91%, 92%, 93%, 94%, 95%, 96%. 97%, 98% or 99% or at least 20-30%, 20-40%, 20-50%, 20-60%, 20-70%, 20-80%, 20-90%, 20-95%, 20-99%, 30-40%, 30-50%, 30-60%, 30-70%, 30-80%, 30-90%, 30-95%, 30-99%, 40-50%, 40-60%, 40-70%, 40-80%, 40-90%, 40-95%, 40-99%, 50-60%, 50-70%, 50-80%, 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

In one aspect, the sense and antisense strands of an encoded siRNA duplex are typically linked by a short spacer sequence leading to the expression of a stein-loop structure termed short hairpin RNA (shRNA). The hairpin is recognized and cleaved by Dicer, thus generating mature siRNA molecules.

In certain embodiments, the encoded siRNA duplexes of the present disclosure suppress (or degrade) target mRNA. Accordingly, the encoded siRNA duplexes can be used to substantially inhibit gene expression in a cell, for example a neuron or astrocyte. In some aspects, the inhibition of gene expression refers to an inhibition by at least about 20%, such as by at least about 30%, 40%, 50%, 60%, 70%. 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. Accordingly, the protein product of the targeted gene may be inhibited by at least about 20%, such as by at least about 30%, 40%, 50%, 60%, 70%, 80%, 85%, 90%, 95% and 100%, or at least 20-30%, 20-40%, 20-50%, 20-60%, 20-70©′0 20-80©′0 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

According to the present disclosure, the siRNA molecules (as canonical structures without being encoded in an AAV vector genome) are designed and tested for their ability in reducing target mRNA levels in cultured cells.

In certain embodiments, the encoded siRNA molecules comprise a miRNA seed match for the guide strand. In another embodiment, the siRNA molecules comprise a miRNA seed match for the passenger strand. In yet another embodiment, the encoded siRNA duplexes or encoded dsRNA targeting the gene of interest do not comprise a seed match for the guide or passenger strand.

In certain embodiments, the encoded siRNA duplexes or encoded dsRNA targeting the gene of interest may have almost no significant full-length off targets for the guide strand. In another embodiment, the encoded siRNA duplexes targeting a gene of interest may have almost no significant full-length off targets for the passenger strand. The encoded siRNA duplexes targeting the gene of interest may have less than 1%, 2%, 3%, 4%, 5%, 6%, 7%, 8%, 9%, 10%, 11%, 12%, 13%, 14%, 15%, 20%, 25%, 30%, 35%, 40%, 45%, 50%, 1-5%, 2-6%, 3-7%, 4-8%, 5-9%, 5-10%, 6-10%, 5-15%, 5-20%, 5-25% 5-30%, 10-20%, 10-30%, 10-40%, 10-50%, 15-30%, 15-40%, 15-45%, 20-40%, 20-50%, 25-50%, 30-40%, 30-50%, 35-50%, 40-50%, 45-50% full-length off targets for the passenger strand. In yet another embodiment, the encoded siRNA duplexes targeting the gene of interest may have almost no significant full-length off targets for the guide strand or the passenger strand. The encoded siRNA duplexes targeting the gene of interest may have less than 1%, 2%, 3%, 4%, 5%, 6%, 7%, 8%, 9%, 10%,11%, 12%, 13%, 14%, 15%, 20%, 25%, 30%, 35%, 40%, 45%, 50%, 1-5%, 7-6%, 3-7%, 4-8%, 5-9%, 5-10%, 6-10%, 5-15%, 5-20%, 5-25% 5-30%, 10-20%, 10-30%, 10-40%, 10-50%, 15-30⁰x©, 15-40⁰x©, 15-45%, 20-40%, 20-50%, 25-50%, 30-40%, 30-50%, 35-50%, 40-50%. 45-50% full-length off targets for the guide or passenger strand.

In certain embodiments, the encoded siRNA duplexes targeting the gene of interest may have high activity in vitro. In another embodiment, the siRNA molecules may have low activity in vitro. In yet another embodiment, the siRNA duplexes or dsRNA targeting the gene of interest may have high guide strand activity and low passenger strand activity in vitro.

In certain embodiments, the siRNA molecules have a high guide strand activity and low passenger strand activity in vitro. The target knock-down (KD) by the guide strand may be at least 60%, 65%, 70%, 75%, 80%, 85%, 90%, 95%, 99%, 99.5% or 100%. The target knock-down by the guide strand may be 60-65%, 60-70%, 60-75%, 60-80%, 60-85%, 60-90%, 60-95%, 60-99%, 60-99.5%, 60-100%, 65-70%, 65-75%, 65-80%, 65-85%, 65-90%, 65-95%, 65-99%, 65-99.5%, 65-100%, 70-75%, 70-80%, 70-85%, 70-90%, 70-95%, 70-99%, 70-99.5%, 70-100%, 75-80%, 75-85%, 75-90%, 75-95%, 75-99%, 75-99.5%, 75-100%, 80-85%, 80-90%, 80-95%, 80-99%, 80-99.5%, 80-100%, 85-90%, 85-95%, 85-99%, 85-99.5%, 85-100%, 90-95%, 90-99%, 90-99.5%, 90-100%, 95-99%, 95-99.5%, 95-100%, 99-99.5%, 99-100% or 99.5-100%. As a non-limiting example, the target knock-down (KD) by the guide strand is greater than 70%.

In certain embodiments, the IC₅₀ of the passenger strand for the nearest off target is greater than 100 multiplied by the IC₅₀ of the guide strand for the target. As a non-limiting example, if the IC₅₀ of the passenger strand for the nearest off target is greater than 100 multiplied by the IC₅₀ of the guide strand for the target then the siRNA molecules is said to have high guide strand activity and a low passenger strand activity in vitro.

In certain embodiments, the 5′ processing of the guide strand has a correct start (n) at the 5′ end at least 75%, 80%, 85%. 90%, 95%, 99% or 100% of the time in vitro or in vivo. As a non-limiting example, the 5′ processing of the guide strand is precise and has a correct start (n) at the 5′ end at least 99% of the time in vitro. As a non-limiting example, the 5′ processing of the guide strand is precise and has a correct start (n) at the 5′ end at least 99% of the time in vivo.

In certain embodiments, the guide to passenger (G:P) (also referred to as the antisense to sense) strand ratio expressed is 1:10, 1:9, 1:8, 1:7, 1:6, 1:5, 1:4, 1:3, 1:2, 1;1, 2:10, 2:9, 2:8, 2:7, 2:6, 2:5, 2:4, 2:3, 2:2, 3:10, 3:9, 3:8, 3:7, 3:6, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 7:3, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 75:75, 30:70, 35:65, 40:60, 45:55, 50:50, 55:45, 60:40, 65:35, 70:30, 75:75, 80:20, 85:15, 90:10, 95:5, or 99:1 in vitro or in vivo. The guide to passenger ratio refers to the ratio of the guide strands to the passenger strands after the excision of the guide strand. For example, an 80:20 guide to passenger ratio would have 8 guide strands to every 2 passenger strands clipped out of the precursor. As a non-limiting example, the guide-to-passenger strand ratio is 80:20 in vitro. As a non-limiting example, the guide-to-passenger strand ratio is 80:20 in vivo. As a non-limiting example, the guide-to-passenger strand ratio is 8:2 r, vitro, As a non-limiting example, the guide-to-passenger strand ratio is 8:2 in vivo. As a non-limiting example, the guide-to-passenger strand ratio is 9:1 in vitro. As a non-limiting example, the guide-to-passenger strand ratio is 9:1 in vivo.

In certain embodiments, the passenger to guide (P:G) (also referred to as the sense to antisense j strand ratio expressed is 1:10, 1:9, 1:8, 1:7, 1:6, 1:5, 1:4, 1:3, 1:2, 1;1, 2:10, 2:9, 2:8, 2:7, 2:6, 2:5, 2:4, 2:3, 2:2, 2:1, 3:10, 3:9, 3:8, 3:7, 3:6, 3:5, 3:4, 3:3, 3:2, 3:1, 4:10, 4:9, 4:8, 4:7, 4:6, 4:5, 4:4, 4:3, 4:2, 4:1, 5:10, 5:9, 5:8, 5:7, 5:6, 5:5, 5:4, 5:3, 5:2, 5:1, 6:10, 6:9, 6:8, 6:7, 6:6, 6:5, 6:4, 6:3, 6:2, 6:1, 7:10, 7:9, 7:8, 7:7, 7:6, 7:5, 7:4, 73, 7:2, 7:1, 8:10, 8:9, 8:8, 8:7, 8:6, 8:5, 8:4, 8:3, 8:2, 8:1, 9:10, 9:9, 9:8, 9:7, 9:6, 9:5, 9:4, 9:3, 9:2, 9:1, 10:10, 10:9, 10:8, 10:7, 10:6, 10:5, 10:4, 10:3, 10:2, 10:1, 1:99, 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, 95:5, or 99:1 in vitro or in vivo. The passenger to guide ratio refers to the ratio of the passenger strands to the guide strands after the excision of the guide strand. For example, an 80:20 passenger to guide ratio would have 8 passenger strands to every 2 guide strands clipped out of the precursor. As a non-limiting example, the passenger-to-guide strand ratio is 80:20 in vivo. As a non-limiting example, the passenger-to-guide strand ratio is 80:20 in vivo. As a non-limiting example, the passenger-to-guide strand ratio is 8:2 in vitro. As a non-limiting example, the passenger-to-guide strand ratio is 8:2 in vivo. As a non-limiting example, the passenger-to-guide strand ratio is 9:1 in vitro. As a non-limiting example, the passenger-to-guide strand ratio is 9:1 in vivo.

In certain embodiments, the integrity of the vector genome encoding the dsRNA is at least 60%, 65%, 70%, 75%, 80%, 85%, 90%, 95%, 99% or more than 99% of the full length of the construct. As a non-limiting example, the integrity of the vector genome is 80% of the full length of the construct.

In certain embodiments, the passenger and/or guide strand is designed based on the method and rules outlined in European Patent Publication No. EP1752536. the contents of which are herein incorporated by reference in their entirety. As a non-limiting example, the 3′-terminal base of the sequence is adenine, thymine or uracil. As a non-limiting example, the 5′-terminal base of the sequence is guanine or cytosine. As a non-limiting, example, the 3′-terminal sequence comprises seven bases rich in one or more bases of adenine, thymine and uracil. As a non-limiting example, the base number is at such a level as causing RNA interference without expressing cytotoxicity.

### Molecular Scaffold

In certain embodiments, the siRNA molecules may be encoded in a modulatory polynucleotide which also comprises a molecular scaffold. As used herein a “molecular scaffold” is a framework or starting molecule that forms the sequence or structural basis against which to design or make a subsequent molecule.

In certain embodiments, the modulatory polynucleotide which comprises the payload (e.g., siRNA, miRNA or other RNAi agent described herein) comprises molecular scaffold which comprises a leading 5′ flanking; sequence which may be of any length and may be derived in whole or in part from wild type microRNA sequence or be completely artificial. A 3′ flanking sequence may mirror the 5′ flanking sequence in size and origin. In certain embodiments, one or both of the 5′ and 3′ flanking sequences are absent.

In certain embodiments the 5′ and 3′ flanking sequences are the same length.

In certain embodiments the 5′ flanking sequence is from 1-10 nucleotides in length, from 5-15 nucleotides in length, from 10-30 nucleotides in length, from 20-50 nucleotides in length, greater than 40 nucleotides in length, greater than 50 nucleotides in length, greater than 100 nucleotides in length or greater than 200 nucleotides in length.

In certain embodiments, the 5′ flanking sequence may be 1, 2. 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, SO, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142., 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208. 209, 210, 211, 212, 213, 214, 215, 216, 217. 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380. 381, 382, 383, 384, 385, 386, 387. 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 44.2, 443, 444, 445, 446, 447, 448, 449, 450, 451, 452, 453, 454, 455, 456, 457, 458, 459, 460. 461., 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides in length.

In certain embodiments the 3′ flanking sequence is from 1-10 nucleotides in length, from 5-15 nucleotides in length, from 10-30 nucleotides in length, from 20-50 nucleotides in length, greater than 40 nucleotides in length, greater than 50 nucleotides in length., greater than 100 nucleotides in length or greater than 200 nucleotides in length.

In certain embodiments, the 3′ flanking sequence may he 1, 2, 3, 4, 5, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118 119, 120, 121, 122, 123, 124, 125, 126, 1.27, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202. 203, 204, 205, 206, 207, 208, 209. 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282. 283, 284, 285, 286, 2.87, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides in length.

In certain embodiments the 5′ and 3′ flanking sequences are the same sequence. In certain embodiments they differ by 2%, 3%, 4%, 5%, 10%, 20% or more than 30% when aligned to each other.

The 3′ flanking sequence may optionally contain one or more CNNC motifs, where “N” represents any nucleotide.

Foaming the stein of a stem loop structure is a minimum of at least one payload sequence. In certain embodiments, the payload sequence comprises at least one nucleic acid sequence which is in part complementary or will hybridize to the target sequence. In certain embodiments, the payload is an siRNA molecule or fragment of an siRNA molecule.

In certain embodiments, the 5′ arm of the stein loop comprises a sense sequence.

In certain embodiments, the 3′ arm of the stem loop comprises an antisense sequence. The antisense sequence, in some instances, comprises a “G” nucleotide at the 5′ most end.

In certain embodiments, the sense sequence may reside on the 3′ arm while the antisense sequence resides on the 5′ arm of the stem of the stem loop structure.

The sense and antisense sequences may be completely complementary across a substantial portion of their length. In certain embodiments, the sense sequence and antisense sequence may be at least 70, 80, 90, 95 or 99% complementary across independently at least 50, 60, 70, 80, 85, 90, 95, or 99% of the length of the strands.

Neither the identity of the sense sequence nor the homology of th e antisense sequence need be 100% complementary to the target.

Separating the sense and antisense sequence of the stem loop structure is a loop (also known as a loop motif). The loop may be of any length, between 4-30 nucleotides, between 4-20 nucleotides, between 4-15 nucleotides, between 5-15 nucleotides, between 6-12 nucleotides, 6 nucleotides, 7, nucleotides, 8 nucleotides, 9 nucleotides, 10 nucleotides, 11 nucleotides, and/or 12 nucleotides.

In certain embodiments, the loop comprises at least one UGUG motif. In certain embodiments, the UGUG motif is located at the 5′ terminus of the loop.

Spacer regions may be present in the modulatory polynucleotide to separate one or more modules from one another. There may he one or more such spacer regions present.

In certain embodiments, a spacer region of between 8-20, i.e., 8, 9, 10, 11, 12, 13, 14, 15, 16, 17, 18, 19, or 20 nucleotides may be present between the sense sequence and a flanking sequence.

In certain embodiments, the spacer is 13 nucleotides and is located between the 5′ terminus of the sense sequence and a flanking sequence. In certain embodiments, a spacer is of sufficient length to form approximately one helical turn of the sequence.

In certain embodiments, a spacer region of between 8-20, i.e., 8, 9, 10, 11, 12, 13, 14, 15, 16, 17, 18, 19, or 20 nucleotides may be present between the antisense sequence and a flanking sequence.

In certain embodiments, the spacer sequence is between 10-13, i.e., 10, 11, 12 or 13 nucleotides and is located between the 3′ terminus of the antisense sequence and a flanking sequence. In certain embodiments, a spacer is of sufficient length to form approximately one helical turn of the sequence.

In certain embodiments, the modulatory polynucleotide comprises in the 5′ to 3′ direction, a 5′ flanking sequence, a. 5′ arm, a loop motif, a 3′ arm and a 3′ flanking sequence. As a non-limiting example, the 5′ arm may comprise a sense sequence and the 3′ aim comprises the antisense sequence. In another non-limiting example. the 5′ arm comprises the antisense sequence and the 3′ arm comprises the sense sequence.

In certain embodiments, the 5′ arm, payload (e.g., sense and/or antisense sequence), loop motif and/or 3′ arm sequence may be altered (e.g., substituting 1 or more nucleotides, adding nucleotides and/or deleting nucleotides). The alteration may cause a beneficial change in the function of the construct (e.g, increase knock-down of the target sequence, reduce degradation of the construct, reduce off target effect, increase efficiency of the payload, and reduce degradation of the payload).

In certain embodiments, the molecular scaffold of the modulatory polynucleotides is aligned in order to have the rate of excision of the guide strand be greater than the rate of excision of the passenger strand. The rate of excision of the guide or passenger strand may be, independently, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99%. As a non-limiting example, the rate of excision of the guide strand is at least 80%. As another non-limiting example, the rate of excision of the guide strand is at least 90%.

In certain embodiments, the rate of excision of the guide strand is greater than the rate of excision of the passenger strand. In one aspect, the rate of excision of the guide strand may be at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or more than 99% greater than the passenger strand.

In certain embodiments, the efficiency of excision of the guide strand is at least 60%, 65%, 70%, 75%, 80%, 85%, 90%, 95%, 99% or more than 99%. As a non-limiting example, the efficiency of the excision of the guide strand is greater than 80%.

In certain embodiments, the efficiency of the excision of the guide strand is greater than the excision of the passenger strand from the molecular scaffold. The excision of the guide strand may be 2, 3, 4, 5, 6, 7, 8, 9, 10 or more than 10 times more efficient than the excision of the passenger strand from the molecular scaffold.

In certain embodiments, the molecular scaffold comprises a dual-function targeting modulatory polynucleotide. As used herein, a dual-function targeting modulatory polynucleotide is a polynucleotide where both the guide and passenger strands knock down the same target or the guide and passenger strands knock down different targets.

In certain embodiments, the molecular scaffold of the modulatory polynucleotides described herein comprise a 5′ flanking region, a loop region and a 3′ flanking region. Non-limiting examples of the sequences for the 5′ flanking region, loop region and the 3′ flanking region which may be used in the molecular scaffolds described herein are shown in Tables 2-4.

TABLE 2
5′ Flanking Regions for Molecular Scaffold

| | | SEQ |
|---|---|---|
| 5′ Flanking | | ID |
| Region Name | 5′ Flanking Region Sequence | NO |
| 5F1 | UUUAUGCCUCAUCCUCUGAGUGCUGAAGGC | 1725 |
| | UUGCUGUAGGCUGUAUGCUG | |
| 5F2 | GUGCUGGGCGGGGGGCGGCGGGCCCUCCCGC | 1726 |
| | AGAACACCAUGCGCUCUUCGGAA | |
| 5F3 | GAAGCAAAGAAGGGGCAGAGGGAGCCCGUG | 1727 |
| | AGCUGAGUGGGCCAGGGACUGGGAGAAGGA | |
| | GUGAGGAGGCAGGGCCGGCAUGCCUCUGCU | |
| | GCUGGCCAGA | |
| 5F4 | GUGCUGGGCGGGGGGCGGCGGGCCCUCCCGC | 1728 |
| | AGAACACCAUGCGCUCUUCGGGA | |
| 5F5 | GUGCUGGGCGGGGGGCGGCGGGCCCUCCCGC | 1729 |
| | AGAACACCAUGCGCUCCACGGAA | |
| 5F6 | GGGCCCUCCCGCAGAACACCAUGCGCUCCAC | 1730 |
| | GGAA | |
| 5F7 | CUCCCGCAGAACACCAUGCGCUCCACGGAA | 1731 |
| 5F8 | GUGCUGGGCGGGGGGCGGCGGGCCCUCCCGC | 1732 |
| | AGAACACCAUGCGCUCCACGGAAG | |
| 5F9 | GUGCUGGGCGGGGGGCGGCGGGCCCUCCCGC | 1733 |
| | AGAACACCAUGCGCUCCUCGGAA | |

TABLE 3
Loop Motif Regions for Molecular Scaffold

| Loop Motif | Loop Motif Region | SEQ ID |
|---|---|---|
| Region Name | Sequence | NO |
| L1 | UGUGACCUGG | 1734 |
| L2 | UGUGAUUUGG | 1735 |
| L3 | UAUAAUUUGG | 1736 |
| L4 | CCUGACCCAGU | 1737 |
| L5 | GUCUGCACCUGUCACUAG | 1738 |
| L6 | GUGACCCAAG | 1739 |
| L7 | GUGGCCACUGAGAAG | 1740 |
| L8 | GUGACCCAAU | 1741 |
| L9 | GUGACCCAAC | 1742 |
| L10 | GUGGCCACUGAGAAA | 1743 |

TABLE 4
3′ Flanking Regions for Molecular Scaffold

| | | SEQ |
|---|---|---|
| 3′ Flanking | | ID |
| Region Name | 3' Flanking Region Sequence | NO |
| 3F1 | AGUGUAUGAUGCCUGUUACUAGCAUUCACA | 1744 |
| | UGGAACAAAUUGCUGCCGUG | |
| 3F2 | CUGAGGAGCGCCUUGACAGCAGCCAUGGGA | 1745 |
| | GGGCCGCCCCCUACCUCAGUGA | |
| 3F3 | CUGUGGAGCGCCUUGACAGCAGCCAUGGGA | 1746 |
| | GGGCCGCCCCCUACCUCAGUGA | |
| 3F4 | UGGCCGUGUAGUGCUACCCAGCGCUGGCUG | 1747 |
| | CCUCCUCAGCAUUGCAAUUCCUCUCCCAUC | |
| | UGGGCACCAGUCAGCUACCCUGGUGGGAAU | |
| | CUGGGUAGCC | |
| 3F5 | GGCCGUGUAGUGCUACCCAGCGCUGGCUGC | 1748 |
| | CUCCUCAGCAUUGCAAUUCCUCUCCCAUCU | |
| | GGGCACCAGUCAGCUACCCUGGUGGGAAUC | |
| | UGGGUAGCC | |
| 3F6 | UCCUGAGGAGCGCCUUGACAGCAGCCAUGG | 1749 |
| | GAGGGCCGCCCCCUACCUCAGUGA | |
| 3F7 | CUGAGGAGCGCCUUGACAGCAGCCAUGGGA | 1750 |
| | GGGCC | |
| 3F8 | CUGCGGAGCGCCUUGACAGCAGCCAUGGGA | 1751 |
| | GGGCCGCCCCCUACCUCAGUGA | |

Any of the regions described in Tables 2-4 may be used in the molecular scaffolds described herein.

In certain embodiments, the molecular scaffold may comprise one or more linkers known in the art. The linkers may separate regions or one molecular scaffold from another. As a non-limiting example, the molecular scaffold may be polycistronic.

In certain embodiments, the modulatory polynucleotide is designed using at least one of the following properties: loop variant, seed mismatch/bulge/wobble variant, stem mismatch, loop variant and basal stem mismatch variant, seed mismatch and basal stem mismatch variant, stem mismatch and basal stem mismatch variant, seed wobble and basal stem wobble variant, or a stem sequence variant.

Introduction into Cells

The encoded siRNA molecules (e.g., siRNA duplexes) of the present disclosure may be introduced into cells by being encoded by the vector genome of an AAV particle. These AAV particles are engineered and optimized to facilitate the entry into cells that are not readily amendable to transfection/transduction. Also, some synthetic viral vectors possess an ability to integrate the shRNA into the cell genome, thereby leading to stable siRNA expression and long-term knockdown of a target gene. In this manner, viral vectors are engineered as vehicles for specific delivery while lacking the deleterious replication and/or integration features found in wild-type virus.

In certain embodiments, the encoded siRNA molecule is introduced into a cell by transfecting, infecting or transducing the cell with an AAV particle comprising nucleic acid sequences capable of producing the siRNA molecule when transcribed in the cell. In certain embodiments, the siRNA molecule is introduced into a cell by injecting into the cell or tissue an AAV particle comprising a nucleic acid sequence capable of producing the siRNA molecule when transcribed in the cell.

In certain embodiments, prior to transfection/transduction, an AAV particle comprising a nucleic acid sequence encoding the siRNA molecules of the present disclosure may be transfected into cells.

Other methods for introducing AAV particles comprising the nucleic acid sequence for the siRNA molecules described herein may comprise photochemical internalization as described in U. S. Patent publication No. 20120264807, the content of which is incorporated herein by reference in its entirety as related to photochemical internalizations, insofar as it does not conflict with the present disclosure.

In certain embodiments, the formulations described herein may contain at least one AAV particle comprising the nucleic acid sequence encoding the siRNA molecules described herein. In certain embodiments, the siRNA molecules may target the gene of interest at one target site. In another embodiment, the formulation comprises a plurality of AAV particles, each AAV particle comprising a nucleic acid sequence encoding a siRNA molecule targeting the gene of interest at a different target site. The gene of interest may be targeted at 2, 3, 4, 5 or more than 5 sites.

In certain embodiments, the AAV particles from any relevant species, such as, but not limited to, human, pig, dog, mouse, rat or monkey may be introduced into cells.

In certain embodiments, the formulated AAV particles may be introduced into cells or tissues which are relevant to the disease to be treated.

In certain embodiments, the formulated AAV particles may be introduced into cells which have a high level of endogenous expression of the target sequence.

In another embodiment, the formulated AAV particles may be introduced into cells which have a low level of endogenous expression of the target sequence.

In certain embodiments, the cells may be those which have a high efficiency of AAV transduction.

In certain embodiments, formulated AAV particles comprising a nucleic acid sequence encoding the siRNA molecules of the present disclosure may be used to deliver siRNA molecules to the central nervous system (e.g., U.S. Pat. No. 6,180,613, the content of which is incorporated herein by reference in its entirety as related to the deliver and therapeutic use of siRNA molecules and AAV particles, insofar as it does not conflict with the present disclosure).

In certain embodiments, the formulated AAV particles comprising a nucleic acid sequence encoding the siRNA molecules of the present disclosure may further comprise a modified capsid comprising peptides from non viral origin. In other aspects, the AAV particle may contain a CNS specific chimeric capsid to facilitate the delivery of encoded siRNA duplexes into the brain and the spinal cord. For example, an alignment of cap nucleotide sequences from AAV variants exhibiting CNS tropism may be constructed to identify variable region (VR) sequence and structure.

In certain embodiments, the formulated AAV particle comprising a nucleic acid sequence encoding the siRNA molecules of the present disclosure may encode siRNA molecules which are polycistronic molecules. The siRNA molecules may additionally comprise one or more linkers between regions of the siRNA molecules.

In certain embodiments, a formulated AAV particle may comprise at least one of the modulatory polynucleotides encoding at least one of the siRNA sequences or duplexes described herein,

In certain embodiments, an expression vector may comprise, from ITR to ITR recited 5′ to 3′, an ITR, a promoter, an intron, a modulatory polynucleotide, a polyA sequence and an ITR.

In certain embodiments, the encoded siRNA molecule may be located downstream of a promoter in an expression vector such as, but not limited to, CMV, U6, H1, CBA or a CBA promoter with a SV40 intron. Further, the encoded siRNA molecule may also be located upstream of the polyadenylation sequence in an expression vector. As a non-limiting example, the encoded siRNA molecule may he located within 1, 2, 3, 4, 5, 6, 7, 8, 9, 10, 11, 12, 13, 14, 15, 16. 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the promoter and/or upstream of the polyadenylation sequence in an expression vector. As a non-limiting example, the encoded siRNA molecule may be located within the first 1%, 2%, 3%, 4%, 5%, 6%, 7©0 8%, 9%, 10%, 15%, 20%, 25% or more than 25% of the nucleotides downstream from the promoter and/or upstream of the polyadenylation sequence in an expression vector, As another non-limiting, example, the encoded siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence in an expression vector.

In certain embodiments, the encoded siRNA molecule may be located upstream of the polyadenylation sequence in an expression vector. Further, the encoded siRNA molecule may be located downstream of a promoter such as, but not limited to, CMV, U6, CBA or a CBA promoter with a SV40 intron in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the promoter and/or upstream of the polyadenylation sequence in an expression vector. As another non-limiting example, the encoded siRNA molecule may he located within 1-5, 1-10, 1-15, 1-20, 1-25, 1-30, 5-10, 5-15, 5-20, 5-25, 5-30. 10-15, 10-20, 10-25, 10-30, 15-20, 15-25, 15-30, 20-25, 20-30 or 25-30 nucleotides downstream from the promoter and/or upstream of the polyadenylation sequence in an expression vector. As a non-limiting example, the encoded siRNA molecule may he located within the first 1%, 2%, 3%, 4%, 5%, 6%, 7%, 8%, 9%, 10%, 15%, 20%, 25% or more than 25% of the nucleotides downstream from the promoter and/or upstream of the polyadenylation sequence in an expression vector. As another non-limiting example, the encoded siRNA molecule may be located with the first 1-5%, 1-10%, 1-15%, 1-20%, 1-25%, 5-10%, 5-15%, 5-20%, 5-25%, 10-15%, 10-20%, 10-25%, 15-20%, 15-25%, or 20-25% downstream from the promoter and/or upstream of the polyadenylation sequence in an expression vector.

In certain embodiments, the encoded siRNA molecule may be located in a scAAV.

In certain embodiments, the encoded siRNA molecule may be located in an ssAAV.

In certain embodiments, the encoded siRNA molecule may be located near the 5′ end of the flip ITR in an expression vector, In another embodiment, the encoded siRNA molecule may be located near the 3′ end of the flip ITR in an expression vector. In yet another embodiment, the encoded siRNA molecule may be located near the 5′ end of the flop ITR in an expression vector. In yet another embodiment, the encoded siRNA molecule may be located near the 3′ end of the flop ITR in an expression vector. In certain embodiments, the encoded siRNA molecule may be located between the 5′ end of the flip ITR and the 3′ end of the flop ITR in an expression vector. In certain embodiments, the encoded siRNA molecule may be located between (e.g., half-way between the 5′ end of the flip ITR and 3′ end of the flop ITR or the 3′ end of the flop ITR and the 5′ end of the flip ITR), the 3′ end of the flip ITR and the 5′ end of the flip ITR in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 nucleotides downstream from the 5′ or 3′ end of an ITR (e.g., Flip or Flop ITR) in an expression vector. As a non-limiting example, the encoded siRNA molecule may be located within 1, 2, 3, 4, 5, 6, 7, 8, 9, 10, 11, 12, 13, 14, 15, 16, 17, 18, 19, 20, 21, :22, 23, 24, 25, 26 27, 28, 29, 30 or more than 30 nucleotides upstream from the 5′ or 3′ end of an ITR (e.g., Flip or Flop ITR) in an expression vector. As another non-limiting example, the encoded siRNA molecule may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 nucleotides downstream from the 5′ or 3′ end of an1′1′R (e.g., Flip or Flop ITR) in an expression vector. As another non-limiting example, the encoded siRNA molecule may he located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30 or 25-30 upstream from the 5′ or 3′ end of an ITR (e.g., Flip or Flop ITR) in an expression vector. As a non-limiting example, the encoded siRNA molecule may be located within the first 1%, 2%, 3%, 4%, 5%, 6%, 7%, 8%, 9%, 10%, 15%, 20%, 25% or more than 25% of the nucleotides upstream from the 5′ or 3′ end of an ITR (e.g., Flip or Flop ITR) in an expression vector. As another non-limiting example, the encoded siRNA molecule may be located with the first 1-5%, 1-10%, 1-15%, 1-20%, 1-25%, 5-10%, 5-15%, 5-70%, 5-25%, 10-15%, 10-70%, 10-75%, 15-70%, 15-25%, or 20-25% downstream from the 5′ or 3′ end of an ITR (e.g., Flip or Flop ITR) in an expression vector.

In certain embodiments, AAV particle comprising the nucleic acid sequence for the siRNA molecules of the present disclosure may be formulated for CNS delivery. Agents that cross the brain blood barrier may be used. For example, some cell penetrating peptides that can target siRNA molecules to the brain blood barrier endothelium may be used to formulate the siRNA duplexes targeting the gene of interest.

In certain embodiments, the formulated AAV particle comprising a nucleic acid sequence encoding the siRNA molecules of the present disclosure may be administered directly to the CNS. As a non-limiting example, the vector comprises a nucleic acid sequence encoding the siRNA molecules targeting the gene of interest.

In specific embodiments, compositions of formulated AAV particles comprising a nucleic acid sequence encoding the siRNA molecules of the present disclosure may be administered in a way which facilitates the vectors or siRNA molecule to enter the central nervous system and penetrate into motor neurons.

In certain embodiments, the formulated AAV particle may be administered to a subject (e.g., to the CNS of a subject via intrathecal administration) in a therapeutically effective amount for the siRNA duplexes or dsRNA to target the motor neurons and astrocytes in the spinal cord and/or brain stem. As a non-limiting example, the siRNA duplexes or dsRNA may reduce the expression of a protein or mRNA,

### II. AAV PRODUCTION

### General Viral Production Process

Viral production cells for the production of rAAV particles generally comprise mammalian cell types. However, mammalian cells present several complications to the large-scale production of rAAV particles, comprising general low yield of viral-particles-per-replication-cell as well as high risks for undesirable contamination from other mammalian biomaterials in the viral production cell. As a result, insect cells have become an alternative vehicle for large-scale production of rAAV particles,

AAV production systems using insect cells also present a range of complications. For example, high-yield production of rAAV particles often requires a lower expression of Rep78 compared to Rep52. Controlling the relative expression of Rep78 and Rep52 in insect cells thus requires carefully designed control mechanisms within the Rep operon. These control mechanisms can comprise individually engineered insect cell promoters, such as ALE1 promoters for Rep78 and Poll-I promoters for Rep52, or the division of the Rep-encoding nucleotide sequences onto independently engineered sequences or constructs. However, implementation of these control mechanisms often leads to reduced rAAV particle yield or to structurally unstable virions.

In another example, production of rAAV particles requires VP1, VP2 and VP3 proteins which assemble to form the AAV capsid. High-yield production of rAAV particles requires adjusted ratios of VP1, VP2 and VP3, which should generally be around 1:1:10, respectively, but can vary from 1-2 for VP1 and/or 1-2 for VP2, relative to 10 VP3 copies. This ratio is important for the quality of the capsid, as too much VP1 destabilizes the capsid and too little VP1 will decrease the infectivity of the vines.

Wild type AAV use a deficient splicing method to control VP1 expression; a weak start codon (ACG) with special surrounding (“Kozak” sequence) to control VP2, and a standard start codon (ATG) for VP3 expression. However, in some baculovirus systems, the mammalian splicing sequences are not always recognized and unable to properly control the production of VP1, VP2 and VP3. Consequently, neighboring nucleotides and the ACG start sequence from VP2 can be used to drive capsid protein production. Unfortunately, for most of the AAV serotypes, this method creates a capsid with a lower ratio of VP1 compared to VP2 (<1 relative to 10 VP3 copies). To more effectively control the production of VP proteins, non-canonical or start codons have been used, like TTG, GTG or CTG. However, these start codons can be considered suboptimal by those in the art relative to the wild type ATG or ACG start codons (See, WO2007046703 and WO2007148971, the content of which is incorporated herein by reference in its entirety as related to production of AAV capsid proteins, insofar as it does not conflict with the present disclosure).

In another example, production of rAAV particles using a baculovirus/SD system generally requires the widely used bacmid-based Baculovirus Expression Vector System (BEVs), which are not optimized for large-scale AAV production. Aberrant proteolytic degradation of viral proteins in the bacmid-based BEVs is an unexpected issue, precluding the reliable large-scale production of AAV capsid proteins using the baculovims/Sf9 system.

There is continued need for methods and systems which allow for effective and efficient large scale (commercial) production of rAAV particles in mammalian and insect cells.

The details of one or more embodiments of the present disclosure are set forth in the accompanying description below. Other features, objects, and advantages of the present disclosure will be apparent from the description, drawings, and the claims. In the description, the singular forms also comprise the plural unless the context clearly dictates otherwise. Unless defined otherwise, all technical and scientific terms used herein have the same meaning as commonly understood by one of ordinary skill in the art to which this present disclosure belongs. In the case of conflict with disclosures incorporated by reference, the present express description will control.

In certain embodiments, the constructs, polynucleotides, polypeptides, vectors, serotypes, capsids formulations, or particles of the present disclosure may be, may comprise, may be modified by, may be used by, may be used for, may be used with, or may be produced with any sequence, element, construct, system, target or process described in one of the following International Publications: WO2016073693, WO2017023724, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each incorporated herein by reference in their entireties, insofar as they do not conflict with the present disclosure.

AAV production of the present disclosure comprises processes and methods for producing AAV particles and viral vectors which can contact a target cell to deliver a payload, e.g. a recombinant viral construct, which comprises a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

The present disclosure provides methods of producing AAV particles or viral vectors by (a) contacting a viral production cell with one or more viral expression constructs encoding at least one AAV capsid protein and/or at least one AAV replication protein, and one or more payload construct vectors, wherein said payload construct vector comprises a payload construct encoding a payload molecule selected from the group consisting of a transgene, a polynucleotide encoding protein, and a modulatory nucleic acid; (I)) culturing said viral production cell under conditions such that at least one AAV particle or viral vector is produced, and (c) isolating said at least one AAV particle or viral vector.

In these methods a viral expression construct may encode at least one structural protein and/or at least one non-structural protein. The structural protein may comprise any of the native or wild type capsid proteins VP1, VP2 and/or VP3 or a chimeric protein. The non-structural protein may comprise any of the native or wild type Rep78, Rep68, Rep52 and/or Rep40 proteins or a chimeric protein.

In certain embodiments, contacting occurs via transient transfection, viral transduction and/or electroporation.

In certain embodiments, the viral production cell is selected from the group consisting of a mammalian cell and an insect cell. In certain embodiments, the insect cell comprises a Spodoptera frugiperda insect cell. In certain embodiments, the insect cell comprises a S19 insect cell. In certain embodiments, the insect cell comprises a Sf21 insect cell.

The payload construct vector of the present disclosure may comprise at least one inverted terminal repeat (ITR) and may comprise mammalian DNA.

Also provided are AAV particles and viral vectors produced according to the methods described herein.

The AAV particles of the present disclosure may be formulated as a pharmaceutical composition with one or more acceptable excipients.

In certain embodiments, an AAV particle or viral vector may be produced by a method described herein.

In certain embodiments, the AAV particles may be produced by contacting a viral production cell (e.g., an insect cell or a mammalian cell) with at least one viral expression construct encoding at least one capsid protein and at least one AAV replication protein, and at least one payload construct vector. The viral production cell may be contacted by transient transfection, viral transduction and/or electroporation. The payload construct vector may comprise a payload construct encoding a payload molecule such as, but not limited to, a transgene, a polynucleotide encoding protein, and a modulatory nucleic acid. The viral production cell can be cultured under conditions such that at least one AAV particle or viral vector is produced, isolated (e.g., using temperature-induced lysis, mechanical lysis and/or chemical lysis) and/or purified (e.g., using filtration, chromatography and/or immunoaffinity purification). As a non-limiting example, the payload construct vector may comprise mammalian DNA.

In certain embodiments, the AAV particles are produced in an insect cell (e.g., Spodoptera frugiperda (Sf9) cell) using the method described herein. As a non-limiting example, the insect cell is contacted using viral transduction which may comprise baculoviral transduction.

In another embodiment, the AAV particles are produced in a inamnialian cell using the method described herein. As a non-limiting example, the mammalian cell is contacted using transient transfection.

In certain embodiments, the viral expression construct may encode at least one structural protein and at least one non-structural protein. As a non-limiting example, the structural protein comprises VP1, VP2 and/or VP3. As another non-limiting example, the non-structural protein comprises Rep78, Rep68, Rep52 and/or Rep40.

In certain embodiments, the AAV particle production method described herein produces greater than 10¹, greater than 10², greater than 10³, greater than 10⁴ or greater than 10⁵ AAV particles in a viral production cell.

In certain embodiments, a process of the present disclosure comprises production of viral particles in a viral production cell using a viral production system which comprises at least one viral expression construct and at least one payload construct. The at least one viral expression construct and at least one payload construct can be co-transfected (e.g. dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, comprising Rep proteins which replicate the payload construct and Cap proteins which assemble to form a capsid that encloses the replicated payload constructs. The resulting AAV particle is extracted from the viral production cells and processed into a pharmaceutical preparation for administration.

Once administered, the AAV particles contact a target cell and enters the cell in an endosome. The AAV particle releases from the endosome and subsequently contacts the nucleus of the target cell to deliver the payload construct. The payload construct, e.g, recombinant viral construct, is delivered to the nucleus of the target cell wherein the payload molecule encoded by the payload construct may he expressed.

In certain embodiments, the process for production of viral particles utilizes seed cultures of viral production cells that comprise one or more baculoviruses (e.g., a Baculoviral Expression Vector (BEV) or a baculovirus infected insect cell (BIIC) that has been transfected with a viral expression construct and a payload construct vector). In certain embodiments, the seed cultures are harvested, divided into aliquots and frozen, and may be used at a later time point to initiate an infection of a naïve population of production cells.

Large scale production of AAV particles may utilize a bioreactor. The use of a bioreactor allows for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined tune point and AAV particles are purified. In another embodiment, the bioreactor is used for continuous production in which a portion of the culture is harvested at an experimentally determined time point fir purification of AAV particles, and the remaining culture in the bioreactor is refreshed with additional growth media components.

AAV viral particles can be extracted from viral production cells in a process which comprises cell lysis, clarification, sterilization and purification. Cell lysis comprises any process that disrupts the structure of the viral production cell, thereby releasing AAV particles. In certain embodiments cell lysis may comprise thermal shock, chemical, or mechanical lysis methods. Clarification can comprise the gross purification of the mixture of lysed cells, media components, and AAV particles. In certain embodiments, clarification comprises centrifugation and/or filtration, comprising but not limited to depth end, tangential flow, and/or hollow fiber filtration.

The end result of viral production is a purified collection of AAV particles which comprise two components: (1) a payload construct (e.g. a recombinant viral genome construct) and (2) a viral capsid.

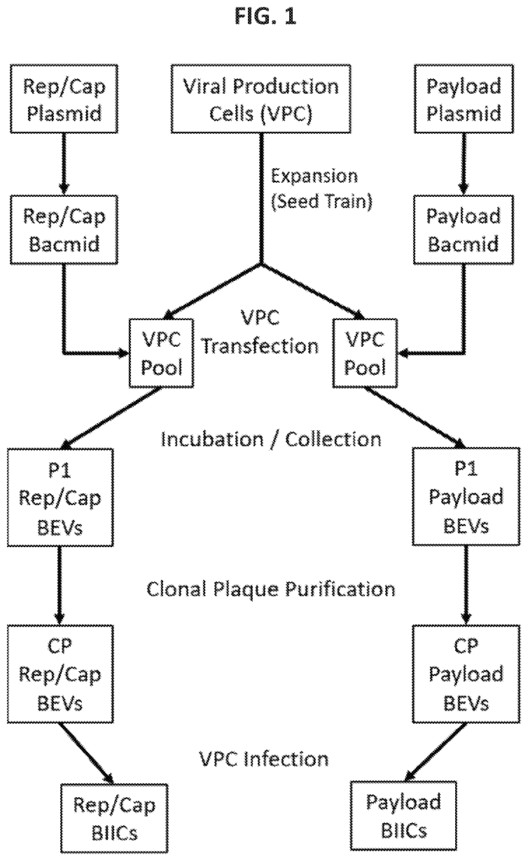

In certain embodiments, such as the embodiment presented in FIG. 1, a viral production system or process of the present disclosure comprises steps for producing baculovirus infected insect cells (BACs) using Viral Production Cells (VPC) and plasmid constructs. Viral Production Cells (VPCs) from a Cell Bank (CB) are thawed and expanded to provide a target working volume and VPC concentration, The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce PI Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can comprise a single CP Purification step or can comprise multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

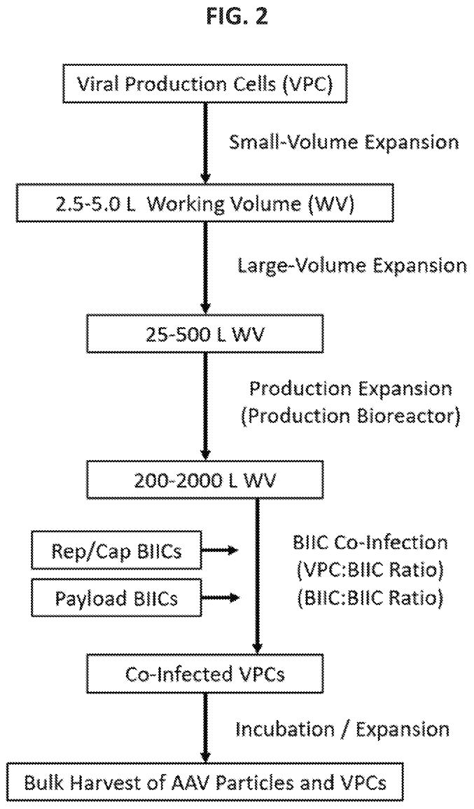

In certain embodiments, such as the embodiment presented in FIG. 2, a viral production system or process of the present disclosure comprises steps for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. The working volume of Viral Production Cells is seeded into a Production Bioreactor and can be further expanded to a working volume of 200-2000 L with a target VPC concentration for BIIC infection. The working volume of VPCs in the Production Bioreactor is then co-infected with Rep/Cap BIICs and Payload BACs, with a target VPC:BIIC ratio and a target BIIC:BIIC ratio. VCD infection can also utilize BEVs. The co-infected VPCs are incubated and expanded in the Production Bioreactor to produce a bulk harvest of AAV particles and VPCs.

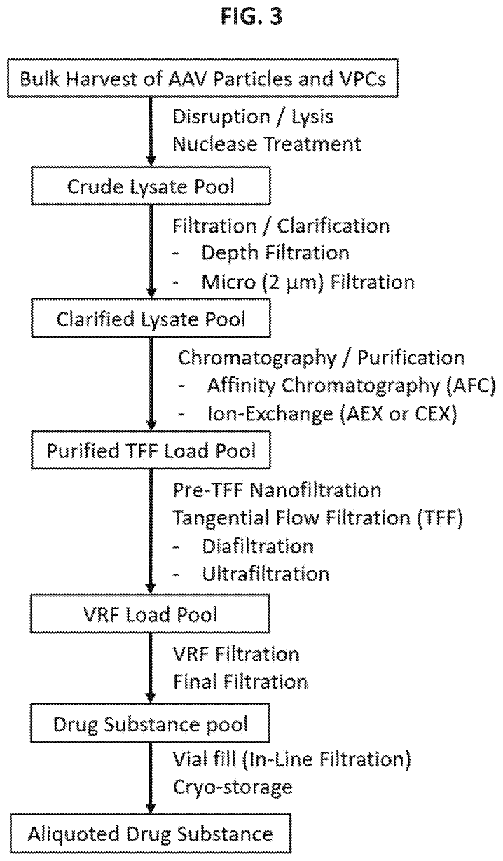

In certain embodiments, such as the embodiment presented in FIG. 3, a viral production system or process of the present disclosure comprises steps for producing a Drug Substance by processing, clarifying and purifying a bulk harvest of AAV particles and Viral Production Cells. A bulk harvest of AAV particles and VPCs (within a Production Bioreactor) are processed through cellular disruption and lysis (e.g, chemical lysis and/or mechanical lysis), followed by nuclease treatment of the lysis pool, thereby producing a crude lysate pool. The crude lysate pool is processed through one or more filtration and clarification steps, comprising depth filtration and microfiltration to provide a clarified lysate pool. The clarified lysate pool is processed through one or more chromatography and. purification steps, comprising affinity chromatography (AFC) and ion-exchange chromatography (AEX or CEX) to provide a purified product pool. The purified product pool is then optionally processed through nanofiltration, and then through tangential flow filtration (TFF). The TFF process comprises one or more diafiltration (DF) steps and one or more ultrafiltration (UF) steps, either in series or alternating. The product pool is further processed through viral retention filtration (VRF) and another filtration step to provide a drug substance pool. The drug substance pool can be further filtered, then aliquoted into vials for storage and treatment.

### Viral Expression Constructs

The viral production system of the present disclosure comprises one or more viral expression constructs which can be transfected./transduced into a viral production cell. In certain embodiments, a viral expression construct or a payload construct of the present disclosure can be a bacmid, also known as a baculovirus plasmid or recombinant baculovirus genotne. In certain embodiments, the viral expression comprises a protein-coding nucleotide sequence and at least one expression control sequence for expression in a viral production cell. In certain embodiments, the viral expression comprises a protein-coding nucleotide sequence operably linked to least one expression control sequence for expression in a viral production cell. In certain embodiments, the viral expression construct contains parvoviral genes under control of one or more promoters. Parvoviral genes can comprise nucleotide sequences encoding non-structural AAV replication proteins, such as Rep genes which encode Rep52, Rep40, Rep68 or Rep78 proteins. Parvoviral genes can comprise nucleotide sequences encoding structural AAV proteins, such as Cap genes which encode VP1, VP2 and VP3 proteins.

The viral production system of the present disclosure is not limited by the viral expression vector used to introduce the parvoviral functions into the virus replication cell. The presence of the viral expression construct in the virus replication cell need not be permanent. The viral expression constructs can be introduced by any means known, for example by chemical treatment of the cells, electroporation, or infection.

Viral expression constructs of the present disclosure may comprise any compound or formulation, biological or chemical, which facilitates transformation, transfection, or transduction of a cell with a nucleic acid. Exemplary biological viral expression constructs comprise plasmids, linear nucleic acid molecules, and recombinant viruses comprising baculovirus. Exemplary chemical vectors comprise lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994.); Maniatis et al., eds. Molecular Cloning. CSH Laboratory, NY, N.Y. (1982); and, Philiport and Scluber, eds. Liposoes as tools in Basic Research and Industry. CRC Press, Ann Arbor, Mich. (1995), the contents of which are each incorporated herein by reference in their entireties as related to viral expression constructs and uses thereof, insofar as they do not conflict with the present disclosure.

In certain embodiments, the viral expression construct is an AAV expression construct which comprises one or more nucleotide sequences encoding non-structural AAV replication proteins, structural AAV capsid proteins, or a combination thereof.

In certain embodiments, the viral expression construct of the present disclosure may be a plasmid vector. In certain embodiments, the viral expression construct of the present disclosure may be a baculoviral construct.

The present disclosure is not limited by the number of viral expression constructs employed to produce AAV particles or viral vectors. In certain embodiments, one, two, three, four, five, six, or more viral expression constructs can be employed to produce AAV particles in viral production cells in accordance with the present disclosure. In one non-limiting example, five expression constructs may individually encode AAV VP1, AAV VP2, AAV VP3, Rep52, Rep78, and with an accompanying payload construct comprising a payload polynucleotide and at least one AAV ITR. In another embodiment, expression constructs may be employed to express, for example, Rep52 and Rep40, or Rep78 and Rep 68. Expression constructs may comprise any combination of VP1, VP2, VP3, Rep52/Rep40, and Rep78/Rep68 coding sequences.

In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, the viral expression construct may encode the components of a Parvoviral capsid with incorporated Gly-Ala repeat region, which may function as an immune invasion sequence, as described in US Patent Application 20110171262, the content of which is incorporated herein by reference in its entirety as related to Parvoviral capsid proteins, insofar as it does not conflict with the present disclosure.

In certain embodiments of the present disclosure, a viral expression construct may be used for the production of AAV particles in insect cells. In certain embodiments, modifications may be made to the wild type AAV sequences of the capsid and/or rep genes, for example to improve attributes of the viral particle, such as increased infectivity or specificity, or to enhance production yields from insect cells.

In certain embodiments, a VP-coding region encodes one or more AAV capsid proteins of a specific AAV serotype. The AAV serotypes for VP-coding regions can be the same or different. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In certain embodiments, a nucleotide sequence encoding one or more VP capsid proteins can be codon optimized to have a nucleotide homology with the reference nucleotide sequence of less than 100%, In certain embodiments, the nucleotide homology between the codon-optimized VP nucleotide sequence and the reference VP nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%. VP-coding region

In certain embodiments, a viral expression construct can comprise a VP-coding region; a VP-coding region is a nucleotide sequence which comprises a VP nucleotide sequence encoding VP1, VP2, VP3, or a combination thereof. in certain embodiments, a viral expression construct can comprise a VP I-coding region; a VPI-coding region is a nucleotide sequence which comprises a VP1 nucleotide sequence encoding a VP1 protein. In certain embodiments, a viral expression construct can comprise a VP2-coding region; a VP2-coding region is a nucleotide sequence which comprises a VP2 nucleotide sequence encoding a VP2 protein. In certain embodiments, a viral expression construct can comprise a VP3-coding region; a VP3-coding region is a nucleotide sequence which comprises a VP3 nucleotide sequence encoding a VP3 protein.

Structural VP proteins, VP1, VP2, and. VP3 of a viral expression construct can be encoded in a single open reading frame regulated by utilization of both alternative splice acceptor and non-canonical translational initiation codons. VP1, VP2 and VP3 can be transcribed and translated from a single transcript in which both in-frame and/or out-of-frame start codons are engineered to control the VP1:VP2:VP3 ratio produced by the nucleotide transcript. In certain embodiments, VP1 can be produced from a sequence which encodes for VP1 only. As use herein, the terms “only for VP1” or “VP1 only” refers to a nucleotide sequence or transcript which encodes for a VP1 capsid protein and: (i) lacks the necessary start codons within the VP1 sequence (i.e. deleted or mutated) for full transcription or translation of VP2 and VP3 from the same sequence; (ii) comprises additional codons within the VP1 sequence which prevent transcription or translation of VP2 and VP3 from the same sequence; or (iii) comprises a start codon for VP1 (e.g. ATG), such that VP1 is the primary VP protein produced by the nucleotide transcript.

In certain embodiments, VP2 can be produced from a sequence which encodes for VP2 only. As use herein, the terms “only for VP2” or “VP2 only” refers to a nucleotide sequence or transcript which encodes for a VP2 capsid protein and: (i) the nucleotide transcript is a truncated variant of a full VP capsid sequence which encodes only VP2 and VP3 capsid proteins; and (ii) which comprise a start codon for VP2 (e.g. ATG), such that VP2 is the primary VP protein produced by the nucleotide transcript.

In certain embodiments, VP1 and VP2 can he produced from a sequence which encodes for VP1 and VP2 only. As use herein, the terns “only for VP1 and VP2” or “VP1 and VP2 only” refer to a nucleotide sequence or transcript which encodes for VP1 and VP2 capsid proteins and: (i) lacks the necessary start codons within the VP sequence (i.e. deleted or mutated) for full transcription or translation of VP3 from the same sequence; (ii) comprises additional codons within the VP sequence which prevent transcription or translation of VP3 from the same sequence; (iii) comprises a start codon for VP1 (e.g. ATG) and VP2 (e.g. ATG), such that VP1 and VP2 are the primary VP protein produced by the nucleotide transcript; or (iv) comprises VP1-only nucleotide transcript and a VP2-only nucleotide transcript connected by a linker, such as an IRES region.

In certain embodiments, the viral expression construct may contain a nucleotide sequence which comprises start codon region, such as a sequence encoding AAV capsid proteins which comprise one or more start codon regions. In certain embodiments, the start codon region can be within an expression control sequence. The start codon can be ATG or a non-ATG codon (i.e., a suboptimal start codon where the start codon of the AAV VP1 capsid protein is a non-ATG). In certain embodiments, the viral expression construct used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in . U.S. Pat. No. 8,163,543, the content of which is incorporated herein by reference in its entirety as related to AAV capsid proteins and the production thereof, insofar as it does not conflict with the present disclosure.

### Rep-Coding Region

In certain embodiments, a viral expression construct can comprise a Rep52-coding region; a Rep52-coding region is a nucleotide sequence which comprises a Rep52 nucleotide sequence encoding a Rep52 protein. In certain embodiments, a viral expression construct can comprise a Rep78-coding region; a Rep78-coding region is a nucleotide sequence which comprises a Rep78 nucleotide sequence encoding a Rep78 protein. In certain embodiments, a viral expression construct can comprise a Rep40-coding region; a Rep40-coding region is a nucleotide sequence which comprises a Rep40 nucleotide sequence encoding a Rep40 protein. In certain embodiments, a viral expression construct can comprise a Rep68-coding region; a Rep68-coding region is a nucleotide sequence which comprises a Rep68 nucleotide sequence encoding a Rep68 protein.

Non-structural proteins, Rep52 and Rep78, of a viral expression construct can be encoded in a single open reading frame regulated by utilization of both alternative splice acceptor and non-canonical translational initiation codons.

Both Rep78 and Rep52 can be translated from a single transcript: Rep78 translation initiates at a first start codon (AUG or non-AUG) and Rep52 translation initiates from a Rep52 start codon AUG) within the Rep78 sequence, Rep78 and Rep52 can also he translated from separate transcripts with independent start codons. The Rep52 initiation codons within the Rep78 sequence can be mutated, modified or removed, such that processing of the modified Rep78 sequence will not produce Rep52 proteins.

In certain embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG and GTG, that effects partial exon skipping upon expression in insect cells, as described in U.S. Pat. No. 8,512,981, the content of which is incorporated herein by reference in its entirety as related to the promotion of less abundant expression of Rep78 as compared to Rep52 to promote high vector yields, insofar as it does not conflict with the present disclosure.

In certain embodiments, the viral expression construct may be a plastnid vector or a baculoviral construct for the expression in insect cells that contains repeating codons with differential codon biases, for example to achieve improved ratios of Rep proteins, e.g. Rep78 and Rep52 thereby improving large scale (commercial) production of viral expression construct and/or payload construct vectors in insect cells, as taught in U.S. Pat. No. 8,697,417, the content of which is incorporated herein by reference in its entirety as related to AAV replication proteins and the production thereof, insofar as it does not conflict with the present disclosure.

In certain embodiment, improved ratios of rep proteins may be achieved using the method and constructs described in U.S. Pat. No. 8,642,314, the content of which is incorporated herein by reference in its entirety as related to AAV replications proteins and the production thereof, insofar as it does not conflict with the present disclosure.

In certain embodiments, the viral expression construct may encode mutant parvoviral Rep polypeptides which have one or more improved properties as compared with their corresponding wild type Rep polypeptide, such as the preparation of higher virus titers for large scale production. Alternatively, they may be able to allow the production of better-quality viral particles or sustain more stable production of virus. in a non-limiting example, the viral expression construct may encode mutant Rep polypeptides with a mutated nuclear localization sequence or zinc finger domain, as described in Patent Application US 20130023034, the content of which is incorporated herein by reference in its entirety as related to AAV replications proteins and the production thereof, insofar as it does not conflict with the present disclosure.

### REN Access Points and Polynucleotide Inserts

In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure (e.g. bacmid) can comprise a polynucleotide incorporated by homologous recombination (transposon donor/acceptor system) into the bacmid by standard molecular biology techniques known and performed by a person skilled in the art,

In certain embodiments, the polynucleotide incorporated into the bacmid (i.e. polynucleotide insert) can comprise an expression control sequence operably linked to a protein-coding nucleotide sequence. In certain embodiments, the polynucleotide incorporated into the bacmid can comprise an expression control sequence which comprises a promoter, such as p10 or polH, and which is operably linked to a nucleotide sequence which encodes a structural AAV capsid protein (e.g. VP1, VP2, VP3 or a combination thereof). In certain embodiments, the polynucleotide incorporated into the bacmid can comprise an expression control sequence which comprises a promoter, such as p10 or polH, and which is operably linked to a nucleotide sequence which encodes a non-structural AAV capsid protein (e.g. Rep78, Rep52, or a combination thereof).

In certain embodiments, the polynucleotide insert can be incorporated into the bacmid at the location of a baculoviral gene. In certain embodiments, the polynucleotide insert can be incorporated into the bacmid at the location of a non-essential baculoviral gene. In certain embodiments, the polynucleotide, insert can be incorporated into the bacmid by replacing a baculoviral gene or a portion of the baculoviral gene with the polynucleotide insert. In certain embodiments, the polynucleotide insert can be incorporated into the bacmid by replacing a baculoviral gene or a portion of the baculoviral gene with a fusion-polynucleotide which comprises the polynucleotide insert and the baculoviral gene (or portion thereof) being replaced.

In certain embodiments, the polynucleotide insert can be incorporated into the bacmid by splitting a baculoviral gene with the polynucleotide insert (i.e. the polynucleotide insert is incorporated into the middle of the gene, separating a 5′-portion of the gene from a 3′-portion of the bacmid gene). In certain embodiments, the polynucleotide insert can be incorporated into the bacmid by splitting a baculoviral gene with the fusion-polynucleotide which comprises the polynucleotide insert and a portion of the baculoviral gene which was split. In certain embodiments, the 3′ end of the fusion-polynucleotide comprises the 5′-portion of the gene that was split, such that the 5′-portion of the gene in the fusion-polynucleotide and the 3′-portion of the gene remaining in the bacmid form a full or functional portion of the baculoviral gene. In certain embodiments, the 5′ end of the fusion-polynucleotide comprises the 3′-portion of the gene that was split, such that the 3′-portion of the gene in the fusion-polynucleotide and the 5′-portion of the gene remaining in the bacmid form a full or functional portion of the baculoviral gene.

In certain embodiments, the polynucleotide can he incorporated into the bacmid at the location of a restriction endonuclease (REN) cleavage site (i.e. REN access point) associated with a baculoviral gene. In certain embodiments, the REN access point in the bacmid is FseI (corresponding with the gta baculovirus gene) (ggccggcc). In certain embodiments, the REN access point in the bacmid is Sdal. (corresponding with the DNA polymerase baculovirus gene) (cctgcagg). In certain embodiments, the REN access point in the bacmid is MauBI (corresponding with the lef-1 baculovirus gene) (cgcgcgcg). In certain embodiments, the REN access point in the bacmid is SbfI (corresponding with the gp64/gp67 baculovirus gene) (cctgcagg). In certain embodiments, the REN access point in the bacmid is I-CeuI (corresponding with the v-cath baculovirus gene) (SEQ ID NO: 1752). In certain embodiments, the REN access point in the bacmid is AvrII (corresponding with the egt baculovirus gene) (cctagg). In certain embodiments, the REN access point in the bacmid is NheI (gctagc). In certain embodiments, the REN access point in the bacmid is SpeI (actagt). In certain embodiments, the REN access point in the bacmid is BstZ17I (gtatac). In certain embodiments, the REN access point in the bacmid is NcoI (ccatgg). In certain embodiments, the REN access point in the bacmid is MluI (acgcgt).

In certain embodiments where the bacmid is a double-stranded construct, the REN cleavage site can comprise a cleavage sequence in one strand and the reverse complement of the cleavage sequence (which also functions as a cleavage sequence) in the other strand. A polynucleotide insert (or strand thereof) can thus comprise a REN cleavage sequence or the reverse complement REN cleavage sequence (which are generally functionally interchangeable). As a non-limiting example, a strand of a polynucleotide insert can comprise an FseI cleave sequence (ggccggcc) or its reverse complement REN cleavage sequence (ccggccgg).

Polynucleotides can be incorporated into these REN access points by: (i) providing a polynucleotide insert which has been engineered to comprise a target REN cleavage sequence (e.g. a polynucleotide insert engineered to comprise FseI REN sequences at both ends of the polynucleotide); (ii) proving a bacmid which comprises the target REN access point for polynucleotide insertion (e.g. a variant of the AcMNPV bacmid bMON14272 which comprises an FseI cleavage site (ii) digesting the REN-engineered polynucleotide with the appropriate REN enzyme (e.g. using FseI enzyme to digesting the REN-engineering polynucleotide which comprises the FseI regions at both ends, to produce a polynucleotide-FseI insert); (iii) digesting the bacmid with the same REN enzyme to produce a single-cut bacmid at the REN access point (e.g. using FseI enzyme to produce a single-cut bacmid at the FseI location); and (iv) ligating the polynucleotide insert into the single-cut bacmid using an appropriate ligation enzyme, such as T4 ligase enzyme. The result is engineered bacmid. DNA which comprises the engineered polynucleotide insert at the target REN access point.

The insertion process can be repeated one or more times to incorporate other engineered polynucleotide inserts into the same bacmid at different REN access points (e.g. insertion of a first engineered polynucleotide insert at the AvrII REN access point in the egt, followed by insertion of a second engineered polynucleotide insert at the I-CeuI REN access point in the cath gene, and followed by insertion of a third enaineered polynucleotide insert at the FseI REN access point in the gta gene).

In certain embodiments, restriction endonuclease (REN) cleavage can be used to remove one or more wild-type genes from a bacmid. In certain embodiments, restriction endonuclease (REN) cleavage can be used to remove one or more engineered polynucleotide insert which has been previously been inserted into the bacmid. In certain embodiments, restriction endonuclease (REN) cleavage can be used to replace one or more engineered polynucleotide inserts with a different enaineered polynucleotide insert which comprises the same REN cleavage sequences (e.g. an engineered polynucleotide insert at the FseI REN access point can be replaced with a different engineered polynucleotide insert which comprises FseI REN cleavage sequences)

### Expression Control

### Expression Control Regions

The viral expression constructs of the present disclosure can comprise one or more expression control region encoded by expression control sequences. In certain embodiments, the expression control sequences are for expression in a viral production cell, such as an insect cell. In certain embodiments, the expression control sequences are operably linked to a protein-coding nucleotide sequence. In certain embodiments, the expression control sequences are operably linked to a VP coding nucleotide sequence or a Rep coding nucleotide sequence.

Herein, the terms “coding nucleotide sequence”, “protein-encoding gene” or “protein-coding nucleotide sequence” refer to a nucleotide sequence that encodes or is translated into a protein product, such as VP proteins or Rep proteins. “Operably linked” means that the expression control sequence is positioned relative to the coding sequence such that it can promote the expression of the encoded gene product.

“Expression control sequence” refers to a nucleic acid sequence that regulates the expression of a nucleotide sequence to which it is operably linked. An expression control sequence is “operably linked” to a nucleotide sequence when the expression control sequence controls and regulates the transcription and/or the translation of the nucleotide sequence. Thus, an expression control sequence can comprise promoters, enhancers, untranslated regions (UIRs), internal ribosome entry sites (IRES), transcription terminators, a start codon in front of a protein-encoding gene, splicing signal for introns, and stop codons. The term “expression control sequence” is intended to comprise, at a minimum, a sequence whose presence are designed to influence expression, and can also comprise additional advantageous components. For example, leader sequences and fusion partner sequences are expression control sequences. The term can also comprise the design of the nucleic acid sequence such that undesirable, potential initiation codons in and out of frame, are removed from the sequence. It can also comprise the design of the nucleic acid sequence such that undesirable potential splice sites arc removed. It comprises sequences or polyadenylation sequences (pA) which direct the addition of a polyA tail, i.e., a string of adenine residues at the 3′-end of an mIRNA, sequences referred to as polyA sequences. It also can be designed to enhance mRNA stability. Expression control sequences which affect the transcription and translation stability, e.g., promoters, as well as sequences which effect the translation, e.g,- Kozak sequences, are known in insect cells. Expression control sequences can be of such nature as to modulate the nucleotide sequence to which it is operably linked such that lower expression levels or higher expression levels are achieved. 104341 In certain embodiments, the expression control sequence can comprise one or more promoters. Promoters can comprise, but are not limited to, baculovirus major late promoters, insect virus promoters, non-insect virus promoters, vertebrate virus promoters, nuclear gene promoters, chimeric promoters from one or more species comprising virus and non-virus elements, and/or synthetic promoters. In certain embodiments, a promoter can be Ctx, Op-EI, EI, ΔEI, EI-1, pH, PIO, polH (polyhedron), ΔpolH, Dmhsp70, Hr1, Hsp70, 4xHsp27 EcRE+minimal Hsp70, IE, IE-1, ΔIE, p10, Δp10 (modified variations or derivatives of p10), p5, p19, p35, p40, p6.9, and variations or derivatives thereof. In certain embodiments, the promoter is a Ctx promoter. In certain embodiments, the promoter is a p10 promoter. In certain embodiments, the promoter is a polH promoter. In certain embodiments, a promoter can be selected from tissue-specific promoters, cell-type-specific promoters, cell-cycle-specific promoters, and variations or derivatives thereof. In certain embodiments, a promoter can be a CMV promoter, an alpha 1-antitrypsin (α1-AT) promoter, a thyroid hormone-binding globulin promoter, a thyroxine-binding globulin (LPS) promoter, an HCR-ApoCII hybrid promoter, an HCR-hAAT hybrid promoter, an albumin promoter, an apolipoprotein E promoter, an α1-AT÷EaIb promoter, a tumor-selective E2F promoter, a mononuclear blood IL-2 promoter, and variations or derivatives thereof. In certain embodiments, the promoter is a low-expression promoter sequence. In certain embodiments, the promoter is an enhanced-expression promoter sequence. In certain embodiments, the promoter can comprise Rep or Cap promoters as described in US Patent Application 20110136227, the content of which is incorporated herein by reference in its entirety as related to expression promoters, insofar as it does not conflict with the present disclosure.

In certain embodiments, a viral expression construct can comprise the same promoter in all nucleotide sequences. In certain embodiments, a viral expression construct can comprise the same promoter in two or more nucleotide sequences. In certain embodiments, a viral expression construct can comprise a different promoter in two or more nucleotide sequences. In certain embodiments, a viral expression construct can comprise a different promoter in all nucleotide sequences.

In certain embodiments the viral expression construct encodes elements to improve expression in certain cell types. In a further embodiment, the expression construct may comprise polh and/or ΔIE-1 insect transcriptional promoters, CMV mammalian transcriptional promoter, and/or p10 insect specific promoters for expression of a desired gene in a mammalian or insect cell.

More than one expression control sequence can be operably linked to a given nucleotide sequence. For example, a promoter sequence, a translation initiation sequence, and a stop codon can be operably linked to a nucleotide sequence.

In certain embodiments, the viral expression construct can comprise one or more expression control sequence between protein-coding nucleotide sequences. In certain embodiments, an expression control region can comprise an IRES sequence region which comprises an IRES nucleotide sequence encoding an internal ribosome entry sight (IRES). The internal ribosome entry sight (IRES) can be selected from the group consisting or: FMDV-IRES from Foot-and-Mouth-Disease virus, EMCV-IRES from Encephalomyocarditis virus, and combinations thereof.

In certain embodiments, an expression control region can comprise a 2A sequence region which comprises a 2A nucleotide sequence encoding a viral 2A peptide. The sequence allows for co-translation of multiple polypeptides within a single open reading frame (ORF). As the ORF is translated, glycine and prolific residues with the 2A sequence prevent the formation of a normal peptide bond, which results in ribosomal “skipping” and “self-cleavage” within the polypeptide chain. The viral 2A peptide can be selected from the group consisting of: F2A from Foot-and-Mouth-Disease virus, T2A from Thosea asigna virus, E2A from Equine rhinitis A virus, P2A from porcine teschovirus-I, BmCPV2A from cytoplasmic polyhedrosis virus, BmIFV 2A from B. mori flacherie virus, and combinations thereof.

In certain embodiments, the viral expression construct may contain a nucleotide sequence which comprises start codon region, such as a sequence encoding AAV capsid proteins which comprise one or more start codon regions. In certain embodiments, the start codon region can be within an expression control sequence.

The method of the present disclosure is not limited by the use of specific expression control sequences. However, when a certain stoichiometry of VP products are achieved (close to 1:1:10 for VP1, VP2, and VP3, respectively) and also when the levels of Rep52 or Rep40 (also referred to as the p19 Reps) are significantly higher than Rep78 or Rep68 (also referred to as the p5 Reps), improved yields of AAV in production cells (such as insect cells) may be obtained. In certain embodiments, the p5/p19 ratio is below 0.6 more, below 0.4, or below 0.3, but always at least 0.03. These ratios can be measured at the level of the protein or can be implicated from the relative levels of specific mRNAs.

In certain embodiments, AAV particles are produced in viral production cells (such as mammalian or insect cells) wherein all three VP proteins are expressed at a stoichiometry approaching, about or Which is 1:1:10 (VP1:VP2:VP3).

In certain embodiments, AAV particles are produced in viral production cells (such as mammalian or insect cells) wherein all three VP proteins are expressed at a stoichiometry approaching, about or which is 2:2:10 (VP1:VP2:VP3).

In certain embodiments, AAV particles are produced in viral production cells (such as mammalian or insect cells) wherein all three VP proteins are expressed at a stoichiometry approaching, about or which is 2:0:10 (VP1:VP2:VP3).

In certain embodiments, AAV particles are produced in viral production cells (such as mammalian or insect cells) wherein all three VP proteins are expressed at a stoichiometry approaching, about or which is 1-2:0-2:10 (VP1:VP2:VP3).

In certain embodiments, AAV particles are produced in viral production cells (such as mammalian or insect cells) wherein all three VP proteins are expressed at a stoichiometry approaching, about or which is 1-2:1-2:10 (VP1:VP2:VP3),

In certain embodiments, AAV particles are produced in viral production cells (such as mammalian or insect cells) wherein all three VP proteins are expressed at a stoichiometry approaching, about or Which is 2-3:0-3:10 (VP1:VP2:VP3).

In certain embodiments, AAV particles are produced in viral production cells (such as mammalian or insect cells) wherein all three VP proteins are expressed at a stoichiometry approaching, about or which is 2-3:2-3:10 (VP1:VP2:VP3).

In certain embodiments, AAV particles are produced in viral production cells (such as mammalian or insect cells) wherein all three VP proteins are expressed at a stoichiometry approaching, about or which is 3:3:10 (VP1:VP2:VP3).

In certain embodiments, AAV particles are produced in viral production cells (such as mammalian or insect cells) wherein all three VP proteins are expressed at a stoichiometry approaching, about or which is 3-5:0-5:10 (VP1: VP2:VP3).

In certain embodiments, AAV particles are produced in viral production cells (such as mammalian or insect cells) wherein all three VP proteins are expressed at a stoichiometry approaching, about or which is 3-5:3-5:10 (VP1:VP2:VP3).

In certain embodiments, the expression control regions are engineered to produce a VP1: VP2:VP3 ratio selected from the group consisting of: about or exactly 1:0:10; about or exactly 1:1:10; about or exactly 2:1:10; about or exactly 2:1:10; about or exactly 2:2:10; about or exactly 3:0:10; about or exactly 3:1:10; about or exactly 3:2:10; about or exactly 3:3:10; about or exactly 4:0:10; about or exactly 4:1:10; about or exactly 4:2:10; about or exactly 4:3:10; about or exactly 4:4:10; about or exactly 5:5:10; about or exactly 1-2:0-2:10; about or exactly 1-2:1-2:10; about or exactly 1-3:0-3:10; about or exactly 1-3:1-3:10; about or exactly 1-4:0-4:10; about or exactly 1-4:1-4:10; about or exactly 1-5:1-5:10; about or exactly 2-3:0-3:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

In certain embodiments of the present disclosure, Rep52 or Rep78 is transcribed from the baculoviral derived polyhedron promoter (polh). Rep52 or Rep78 can also be transcribed from a weaker promoter, for example a deletion mutant of the IE-1 promoter, the ΔIE-1 promoter, has about 20% of the transcriptional activity of that IE-1 promoter. A promoter substantially homologous to the ΔIE-1 promoter may be used. In respect to promoters, a homology of at least 50%, 60%, 70%, 80%, 90% or more, is considered to be a substantially homologous promoter.

### Engineered Untranslated Regions (UTRs)

The present disclosure presents enaineered untranslated regions (UTRs), comprising engineered UTR polynucleotides that function as a 5′ UTR, Engineering the features in untranslated regions (UTRs) can improve the stability and protein production capability of the viral production constructs of the present disclosure.

The present disclosure presents viral expression constructs which comprise an engineered untranslated region (UTR) of the present disclosure. In certain embodiments, the viral expression construct comprises an engineered untranslated region (UTR) of the present disclosure. In certain embodiments, the viral expression construct comprises an engineered 5′ UTR of the present disclosure.

Natural 5′ UTRs comprise features which play important roles in translation initiation. They harbor signatures such as a Kozak sequences which are known to be involved in the process by which the ribosome initiates translation of many genes. The present disclosure provides engineered polynucleotide sequences which comprise at least one 5′ UTR function. Such “engineered 5′ UTR polynucleotides” or “engineered 5′ UTRs” may also comprise the start codon of the protein whose expression is being driven, e.a., a structural AAV capsid protein (VP1, VP2 or VP3) or a non-structural AAV replication protein (Rep78 or Rep52).

According to the present disclosure, the engineered 5′ UTR polynucleotides may range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides). Non-UTR sequences may be incorporated into the engineered 5′ UTRs. For example, introns or portions of introns sequences may be incorporated into the polynucleotides of the disclosure. Incorporation of intronic sequences may also increase AAV serotype protein (e.g., capsid) production.

Leader sequences may be comprised in the engineered polynucleotides. Such leader sequences may derive from or be identical to all or a portion of any AAV serotype selected from those taught herein.

According to the present disclosure, the polynucleotides may comprise a consensus sequence which is discovered through rounds of experimentation. As used herein a “consensus” sequence is a single sequence which represents a collective population of sequences allowing for variability at one or more sites.

In certain embodiments, variants of the polynucleotides of the disclosure may be generated. These variants may have the same or a similar activity as the reference polynucleotide. Alternatively, the variant may have an altered activity (e.g., increased or decreased) relative to a reference polynucleotide. Generally, variants of a particular polynucleotides of the disclosure will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide as determined by sequence alignment programs and parameters described herein and known to those skilled in the art. Such tools for alignment comprise those of the BLAST suite (Stephen F. Altschul, Thomas L. Madden, Alejandro A. Schäffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.) Other tools are described herein, specifically in the definition of “identity.”

The engineered polynucleotides of the present disclosure may be incorporated into a vector or plasmid alone or in combination with other polynucleotide sequences or features such as those disclosed in International Publications WO2007046703 and WO2007148971 (disclosing alternative start codons and AAV vectors produced in insect cells); WO2009104964 (disclosing optimization of expression of AAV proteins in insect cells and involving alteration of promoter strength, enhancer elements, temperature control); and WO2015137802 (disclosing alternative start codons, removal of start codons and AAV vectors produced in insect cells), the contents of which are each incorporated herein by reference in their entireties, insofar as they do not conflict with the present disclosure.

In certain embodiments, the engineered 5′ UTR comprises or consists of between 80-120 nucleotides, between 90-110 nucleotides, between 95-105 nucleotides, between 98-100 nucleotides, or about 99 nucleotides. In certain embodiments, the engineered 5′ UTR comprises or consists of 24 nucleotides.

In certain embodiments, the engineered 5′ UTR is derived from AAV2. In certain embodiments, the engineered 5′ UTR is derived from AAV2. In certain embodiments, the engineered 5′ UTR is derived from AAV9. In certain embodiments, the engineered 5′ UTR is derived from AAVRh10. In certain embodiments, the engineered 5′ UTR is derived from AAVPHP.B. In certain embodiments, the engineered 5′ UTR is derived from an AAV serotype presented in Table 1.

### 5′ UTR Hairpin Structure

In certain embodiments, the engineered 5′ UTR comprises a hairpin structure. In certain embodiments, the engineered 5′ U′IR region comprises: a promoter 5′ (upstream) of a 5′ UTR which comprises an “A” region (a 5′ flanking region) which is 5′ (upstream) of a hairpin, a “B” region (a 3′ flanking region which can comprise a start codon and kozak nucleotides around the start codon) which is 3′ (downstream) from the stem loop, a “C” region representing the stem of a stein-loop structure, and a loop (which can range from 4-16 nucleotides). In certain embodiments, the hairpin structure can comprise all or a portion of a Kozak sequence, such as TTT. The promoter and 5′ UTR can be associated with either a CAP gene (which encodes the structural capsid proteins VP1, VP2 and/or VP3) or a REP gene (which encodes the non-structural replication proteins Rep78 and Rep52).

In certain embodiments, the engineered 5′ UTR comprises a hairpin structure encoded by a hairpin nucleotide sequence. In certain embodiments, the hairpin nucleotide sequence comprises a leader sequence. In certain embodiments, the hairpin nucleotide sequence comprises a leader sequence and a start codon (e.g. ATG). In certain embodiments, the hairpin nucleotide sequence comprises a leader sequence, and a start codon (e.g. ATG) within a Kozak sequence or modified Kozak sequence.

In certain embodiments, the engineered 5′ UTR comprises a hairpin structure, having a 5′ flanking region (i.e. upstream region) encoded by a 5′ flanking sequence. In certain embodiments, the 5′ flanking sequence may be of any length and may be derived in whole or in part from wild type AAV sequence or be completely artificial,

In certain embodiments, the engineered 5′ VTR. comprises a hairpin structure having a 3′ flanking region (i.e. downstream region) encoded by a 3′ flanking sequence. In certain embodiments, the 3′ flanking sequence may be of any length and may be derived in whole or in part from wild type AAV sequence or be completely artificial.

The 5′ flanking sequence and 3′ flanking sequence can have the same size and origin, a different size, a different origin, or a different size and origin. Either flanking sequence may be absent. The 5′ flanking sequence can comprise or consist of 2-50, 2-40, 2-30, 2-20, or 2-15 nucleotides. The 3′ flanking sequence can comprise or consist of 2-50, 2-40, 2-30, 2-20, or 2-15 nucleotides. The 3′ flanking sequence may optionally contain the start codon of an AAV protein or proteins as well as other sequences such as a Kozak or modified Kozak sequence.

In certain embodiments, the engineered 5′ UTR comprises a hairpin structure which comprises a step-loop structure. In certain embodiments, the hairpin structure comprises a stem region and a loop region. In certain embodiments, the hairpin structure comprises a stem region, a loop region, and a stem-complement region. The stem-loop structure can comprise a stein region encoded by a stem sequence. The stein-loop structure can comprise a loop region encoded by a loop sequence. The stem-loop structure can comprise a stem-complement region encoded by a stem-complement sequence. Forming the stem of the stem-loop structure of the hairpin are paired or substantially paired nucleobases of between 2 and 50 pairs. The stein may contain one or more mismatches, bulges or loops. In certain embodiments, the stem sequence and the stem-complement sequence are 100% complementary (i.e. zero mismatches). In certain embodiments, the stem sequence and the stem-complement sequence comprise zero, one, two, three, four or five mismatches.

In certain embodiments, the engineered 5′ UTR comprises a hairpin structure presented in Table 5, or a combination of Upstream, Stem (Upstream), Loop, Stem (Downstream) and/or Downstream components listed in Table 5. In The position of the loop portion of the hairpin is underlined in the sequence with the canonical ATG start codon in Capital Letters.

TABLE 5
5′ UTR Hairpin Structures

| | Hairpin | | | | | |
|---|---|---|---|---|---|---|
| Name | Sequence | Upstream | Stem-U | Loop | Stem-D | Downstream |
| HP1 | atacgactcgacgaaga | atacgactcgacgaag | aaccgtcggc | tttatggct | | |
| | cttgatcaaccgtcggct | acttgatc | SEQ ID NO: | | | |
| | ttATGgct | SEQ ID NO: 1769 | 1773 | | | |
| | SEQ ID NO: 1753 | | | | | |
| HP2 | atacgactcgacgaaga | atacgactcgacgaag | aaccAtcggc | tttatggct | | |
| | cttgatcaaccAtcggc | acttgatc | SEQ ID NO: | | | |
| | tttATGgct | SEQ ID NO: 1769 | 1774 | | | |
| | SEQ ID NO: 1754 | | | | | |
| HP3 | atacgactcgacgaaga | atacgactcgacgaag | aaccgtAggc | tttatggct | | |
| | cttgatcaaccgtAggc | acttgatc | SEQ ID NO: | | | |
| | tttATGgct | SEQ ID NO: 1769 | 1775 | | | |
| | SEQ ID NO: 1755 | | | | | |
| HP4 | atacgactcgacgaaga | atacgactcgacgaag | cggc | tttatggct | | |
| | cttgatcctgactcggct | acttgatcctgact | | | | |
| | ttATGgct | SEQ ID NO: 1770 | | | | |
| | SEQ ID NO: 1756 | | | | | |
| HP5 | atacgactcgacgaaga | atacgactcgacgaag | agTtaaccgtc | tttatggct | | |
| | cttagTtaaccgtcggc | actt | ggc | | | |
| | tttATGgct | SEQ ID NO: 1771 | SEQ ID NO: | | | |
| | SEQ ID NO: 1757 | | 1776 | | | |
| HP6 | atacgactcgacgaaga | atacgactcgacgaag | agTtaaccgtc | tttatggct | | |
| | cttagTtaccgtcCgc | actt | Cgc | | | |
| | tttATGgct | SEQ ID NO: 1771 | SEQ ID NO: | | | |
| | SEQ ID NO: 1758 | | 1777 | | | |
| HP7 | atacgactcgacgaaga | atacgactcgacgaag | agTtaacTgtc | tttatggct | | |
| | cttagTtaacTgtcCg | actt | Cgc | | | |
| | ctttATGgct | SEQ ID NO: 1771 | SEQ ID NO: | | | |
| | SEQ ID NO: 1759 | | 1778 | | | |
| HP8 | atacgactcgacgaaga | atacgactcgacgaag | ctgcc | atctaa | ggcag | tttatggct |
| | cctgccatctaaggcag | ac | | | | |
| | tttATGgct | SEQ ID NO: 1772 | | | | |
| | SEQ ID NO: 1760 | | | | | |
| HP9 | atacgactctgccagct | atacgact | ctgccagctc | atctaa | gagctggcag | tttatggct |
| | catctaagagctggcag | | SEQ ID NO: | | SEQ ID NO: | |
| | tttATGgct | | 1779 | | 1784 | |
| | SEQ ID NO: 1761 | | | | | |
| HP10 | atacgactctgccTgct | atacgact | ctgccTgctc | atctaa | gagctggcag | tttatggct |
| | catctaagagctggcag | | SEQ ID NO: | | SEQ ID NO: | |
| | tttATGgct | | 1780 | | 1784 | |
| | SEQ ID NO: 1762 | | | | | |
| HP11 | atacctgccagctcttcg | atac | ctgccagctcttc | atctaa | cgttagagctgg | tttatggct |
| | atctaacgaagagctgg | | g | | cag | |
| | cagtttATGgct | | SEQ ID NO: | | SEQ ID NO: | |
| | SEQ ID NO: 1763 | | 1781 | | 1785 | |
| HP12 | atacctgccTgctcttc | atac | ctgccTgctctt | atctaa | cgaagagagg | tttatggct |
| | gatctaacgaagagctg | | cg | | cag | |
| | gcagtttATGgct | | SEQ ID NO: | | SEQ ID NO: | |
| | SEQ ID NO: 1764 | | 1782 | | 1785 | |
| HP13 | atacctgccTgctcAtc | atac | ctgccTgctcA | atctaa | cgaagagctgg | tttatggct |
| | gatctaacgaagagctg | | tcg | | cag | |
| | gcagtttATGgct | | SEQ ID NO: | | SEQ ID NO: | |
| | SEQ ID NO: 1765 | | 1783 | | 1785 | |
| HP14 | atacctgccatctaagg | atac | ctgcc | atctaa | ggcag | gactcgacgaag |
| | caggactcgacgaaga | | | | | actttatggct |
| | ctttATGgct | | | | | SEQ ID NO: |
| | SEQ ID NO: 1766 | | | | | 1786 |
| HP15 | atacctgccagctcatct | atac | ctgccagctc | atctaa | gagctggcag | gacttttatggct |
| | aagagctggcaggactt | | SEQ ID NO: | | SEQ ID NO: | SEQ ID NO: |
| | ttATGAct | | 1779 | | 1784 | 1787 |
| | SEQ ID NO: 1767 | | | | | |
| HP16 | atacctgccTgctcatct | atac | ctgccTgctc | atctaa | gagctggcag | gacttttatggct |
| | aagagctggcaggactt | | SEQ ID NO: | | SEQ ID NO: | SEQ ID NO: |
| | ttATGgct | | 1780 | | 1784 | 1787 |
| | SEQ ID NO: 1768 | | | | | |

In certain embodiments, the engineered 5′ UTR. comprises a hairpin structure, or a component thereof, which is encoded by a hairpin nucleotide sequence selected from SEQ ID NO:1753-1768. In certain embodiments, the engineered 5′ UTR comprises a hairpin structure, or a component thereof, encoded by a nucleotide sequence having at least 60% identity, at least 65% identity, at least 70% identity, at least 75% identity, at least 80% identity, at least 85% identity, at least 90% identity or at least 95% identity to SEQ ID NO:1753-1768.

### G: C Content

In certain embodiments, the engineered 5′ UTRs of the present disclosure can comprise a nucleotide sequence, such as a leader sequence, which has varied G:C content or percentage. In certain embodiments, the 5′ flanking region of the 5′ UIRs have a varied G:C content. In certain embodiments, the stem of the 5′ UTRs has a varied (3:C content. In certain embodiments, the 3′ flanking region of the 5′ UTRs has a varied G:C content. In certain embodiments, the G:C content of the engineered 5′ UTR is 10-80%, 20-70%, 25%-65%, or 30%-60%. In certain embodiments, the G:C content of the engineered 5′ UTR is about 25%, about 30%, 34%, about 35%, about 40%, about 45%, about 50%, about 55%, 58%, about 60%, 62% or about 65%.

In certain embodiments, the engineered 5′ VTR comprises or consists of between 98-100 nucleotides, and comprises a G:C content of about 25%, about 30%, 34%, about 35%, about 40%, about 45%, about 50%, about 55%, 58%, about 60%, 62% or about 65%.

### Modified Kozak Sequences

The translational start site of eukaryotic mRNA is controlled in part by a nucleotide sequence referred to as a Kozak sequence as described in Kozak, M Cell. 1986 Jan. 31; 440:283-92 and Kozak, M. J Cell Biol. 1989 February; 108(2):229-41 the content of which is incorporated herein by reference in its entirety as related to Kozak sequences and. uses thereof, insofar as it does not conflict with the present disclosure. Both naturally occurring and synthetic (i.e. modified or engineered) translational start sites of the Kozak form can be used in the production of polypeptides by molecular genetic techniques, as described in Kozak, M. Mamm Genome. 1946 August; 7(8): 563-74, the content of which is incorporated herein by reference in its entirety as related to Kozak sequences and uses thereof, insofar as it does not conflict with the present disclosure. The Kozak consensus sequence is generally defined as GCCRCC(NNN)GC (SEQ. ID NO: 1788), wherein R is a purine (i.e. A or G) and wherein (NNN) stands for a translation initiation start codon, such as a suboptimal start codon. In certain embodiments, Kozak sequences are modified to provide leaky ribosome scanning of the VP-coding region. Any corresponding increase of VP1 production will conversely reduce VP3 translation, such that marginal changes of VP1 initiation rate can induce large changes in VPI/VP3 ratio. As used herein, the terms “modified Kozak sequence” or “engineered Kozak sequence” represent an altered Kozak sequence, such as, for example, a Kozak sequence which comprises nucleotide mutations, additions, or deletions.

In certain embodiments, the engineered 5′ UTRs of the present disclosure can comprise a modified Kozak sequence, such as a modified weak Kozak sequence. In certain embodiments, the engineered 5′ UTRs of the present disclosure can comprise a modified Kozak sequence which comprises or is associated with a VP start codon and/or VP translation initiation region. In certain embodiments, the engineered 5′ UTRs of the present disclosure can comprise a modified Kozak sequence which comprises or is associated with a VP1 start codon and/or VP1 translation initiation region. In certain embodiments, the engineered 5′ UTRs of the present disclosure can comprise a modified Kozak sequence which comprises or is associated with a VP2 start codon and/or VP2 translation initiation region. In certain embodiments, the engineered 5′ UTRs of the present disclosure can comprise a modified Kozak sequence which comprises or is associated with a VP3 start codon and/or VP3 translation initiation region.

In certain embodiments, the engineered 5′ UTRs of the present disclosure can comprise a modified Kozak sequence selected from Table 6.

TABLE 6
Modified Kozak Sequences

| Sequence | SEQ ID NO: |
|---|---|
| tttagatggct | 1789 |
| tttagatgttg | 1790 |
| tttttatgttg | 1791 |

In certain embodiments, the engineered 5′ UTRs of the present disclosure can comprise a modified Kozak sequence selected from SEQ ID NOS: 1789-1791. In certain embodiments, the engineered 5′ UTRs of the present disclosure can comprise a modified Kozak sequence which has at least 60% identity, at least 65% identity, at least 70% identity, at least 75% identity, at least 80% identity, at least 85% identity, at least 90% identity or at least 95% identity to a modified Kozak sequence selected from SEQ ID NOS: 1789-1791.

In certain embodiments, an engineered 5′ UTR. of the present disclosure can comprise the modified Kozak sequence of SEQ ID NO: 1789, In certain embodiments, an engineered 5′ UTR of the present disclosure can comprise the modified Kozak sequence of SEQ ID NO: 1790. In certain embodiments, an engineered 5′ UTR of the present disclosure can comprise the modified Kozak sequence of SEQ ID NO: 1791.

In certain embodiments, the modified Kozak sequence is engineered or selected to produce a VP1:VP2:VP3 ratio selected from: about or exactly 1:1:10; about or exactly 2:2:10; about or exactly 3:3:10; about or exactly 4:4:10; about or exactly 5:5:10; about or exactly 1-2:1-2:10; about or exactly 1-3:1-3:10; about or exactly 1-4:1-4:10; about or exactly 1-5:1-5:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

In certain embodiments, the present disclosure presents method of producing rAAV particles in a viral production cell such as an insect cell. In certain embodiments, the method comprises (i) transfecting a viral production cell (e.g. Sf insect cell) with a payload construct and viral expression construct which comprises a nucleotide sequence encoding a modified Kozak sequence and a sequence encoding VP1, VP2, and/or VP3 capsid proteins, and (ii) culturing the insect cell under conditions suitable to produce rAAV particles.

### Viral Production Cells and Vectors

### Mammalian Cells

Viral production of the present disclosure disclosed herein describes processes and methods for producing AAV particles or viral vector that contacts a target cell to deliver a payload construct, e.g. a recombinant AAV particle or viral construct, which comprises a nucleotide encoding a payload molecule. The viral production cell may be selected from any biological organism, comprising prokaryotic (e. g., bacterial) cells, and eukaryotic cells, comprising, insect cells, yeast cells and mammalian cells.

In certain embodiments, the AAV particles of the present disclosure may be produced in a viral production cell that comprises a mammalian cell. Viral production cells may comprise mammalian cells such as A549, WEH1, 3T3, 1.0T1/2, BHK, MDCK, COS 1, COS 7, BSC 1, BSC 40, BMT 10, VERO. W138, HeLa, HEK293, HEK293T (293T), Saos, C2C12, L cells, HT1080, HepG2 and primary fibroblast, hepatocyte and myoblast cells derived from mammals. Viral production cells can comprise cells derived from mammalian species comprising, but not limited to, human, monkey, mouse, rat, rabbit, and hamster or cell type, comprising but not limited to fibroblast, hepatocyte, tumor cell, cell line transformed cell, etc.

AAV viral production cells commonly used for production of recombinant AAV particles comprise, but is not limited to HEK293 cells. COS cells, C127, 3T3, CHO, HeLa cells, KB cells, BHK, and other mammalian cell lines as described in U.S. Pat. Nos. 6,156,303, 5,387,484, 5,741,683, 5,691,176, 6,428,988 and 5,688,676, U.S. patent application 2002/0081721, and International Patent Publication Nos. WO 00/47757, WO 00/24916, and WO 96/17947, the contents of which are each incorporated herein by reference in their entireties, insofar as they do not conflict with the present disclosure. In certain embodiments, the AAV viral production cells are trans-complementing packaging cell lines that provide functions deleted from a replication-defective helper virus, e.g., HEK293 cells or other Ea trans-complementing cells.

In certain embodiments, the packaging cell line 293-10-3 (ATCC Accession No. PTA-2361) may be used to produce the AAV particles, as described in U.S. Pat. No. 6,281,010, the content of which is incorporated herein by reference in its entirety as related to the 293-10-3 packaging cell line and uses thereof, insofar as it does not conflict with the present disclosure.

In certain embodiments, of the present disclosure a cell line, such as a HeLA cell line, for trans-complementing E1 deleted adenoviral vectors, which encoding adenovirus E1a and adenovirus E1b under the control of a phosphoglycerate kinase (PGK) promoter can be used for AAV particle production as described in U.S. Pat. No. 6,365,394, the content of which is incorporated herein by reference in its entirety as related to the HeLA cell line and uses thereof, insofar as it does not conflict with the present disclosure.

In certain embodiments, AAV particles are produced in mammalian cells using a triple transfection method wherein a payload construct, parvoviral Rep and parvoviral Cap and a helper construct are comprised within three different constructs. The triple transfection method of the three components of AAV particle production may be utilized to produce small lots of virus for assays comprising transduction efficiency, target tissue (tropism) evaluation, and stability.

AAV particles to be formulated may be produced by triple transfection or baculovirus mediated virus production, or any other method known in the art. Any suitable permissive or packaging cell known in the art may be employed to produce the vectors. In certain embodiments, trans-complementing packaging cell lines are used that provide functions deleted from a replication-defective helper virus, e.g., 293 cells or other E1a trans-complementing cells.

The gene cassette may contain some or all of the parvovirus (e.g., AAV) cap and rep genes. In certain embodiments, some or all of the cap and rep functions are provided in trans by introducing a packaging vector(s) encoding the capsid and/or Rep proteins into the cell. In certain embodiments, the gene cassette does not encode the capsid or Rep proteins. Alternatively, a packaging cell line is used that is stably transformed to express the cap and/or rep genes.

Recombinant AAV virus particles are, in certain embodiments, produced and purified from culture supernatants according to the procedure as described in US2016/0032254, the content of which is incorporated herein by reference in its entirety as related to the production and processing of recombinant AAV virus particles, insofar as it does not conflict with the present disclosure. Production may also involve methods known in the art comprising those using 293T cells, triple transfection or any suitable production method.

In certain embodiments, mammalian viral production cells (e.g. 293T cells) can be in an adhesion/adherent state (e.g. with calcium phosphate) or a suspension state (e.g. with polyethyleneimine (PEI). The mammalian viral production cell is transfected with plasmids required for production of AAV, (i.e., AAV rep/cap construct, an adenoviral helper construct, and/or ITR flanked payload construct). In certain embodiments, the transfection process can comprise optional medium changes (e.g. medium changes for cells in adhesion form, no medium changes for cells in suspension form, medium changes for cells in suspension form if desired). In certain embodiments, the transfection process can comprise transfection mediums such as DMEM or F17. In certain embodiments, the transfection medium can comprise serum or can be scrum-free (e.g. cells in adhesion state with calcium phosphate and with serum, cells in suspension state with PEI and without serum).

Cells can subsequently be collected by scraping (adherent form) and/or pelleting (suspension form and scraped adherent form) and transferred into a receptacle. Collection steps can be repeated as necessary for full collection of produced cells. Next, cell lysis can be achieved by consecutive freeze-thaw cycles (−80C to 37C), chemical lysis (such as adding detergent triton), mechanical lysis, or by allowing the cell culture to degrade after reaching ˜0% viability. Cellular debris is removed by centrifugation and/or depth filtration. The samples are quantified for AAV particles by DNase resistant genome titration by DNA qPCR.

AAV particle titers are measured according to genome copy number (genome particles per milliliter). Genome particle concentrations are based on DNA qPCR of the vector DNA as previously reported (Clark et al. (1999) Hum. Gene Ther., 10:1031-1039: Veldwijk et al. (2002) Mol. Ther., 6:272-278, the contents of which are each incorporated herein by reference in their entireties as related to the measurement of particle concentrations, insofar as they do not conflict with the present disclosure)

### Insect Cells

Viral production of the present disclosure comprises processes and methods for producing AAV particles or viral vectors that contact a target cell to deliver a payload construct, e.g. a recombinant viral construct, which comprises a nucleotide encoding a payload molecule. In certain embodiments, the AAV particles or viral vectors of the present disclosure may be produced in a viral production cell that comprises an insect cell.

Growing conditions for insect cells in culture, and production of heterologous products in insect cells in culture are well-known in the art, see U.S. Pat. No. 6,204,059, the content of which is incorporated herein by reference in its entirety as related to the growth and use of insect cells in viral production, insofar as it does not conflict with the present disclosure.

Any insect cell which allows for replication of parvovirus and which can he maintained in culture can be used in accordance with the present disclosure. AAV viral production cells commonly used for production of recombinant AAV particles comprise, but is not limited to, Spodoptera frugiperda, comprising, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, NJ (1995); O′Reilly et al., Baculovirus Expression Vectors, A Laboratory Manual, Oxford Univ. Press (1994); Satnulski et al., J. Vir.63:3822-8 (1989); Kajigaya et al., Proc. Nat'l. Acad. Sci. USA 88: 4646-50 (1991); Ruffin et al., J. Vir. 66:6922-30 (1992); Kimbauer et al ., Vir.21.9:37-44 (1996); Zhao et al., Vir.272:382-93 (2000); and Samulski et al., U.S. Pat. No. 6,204,059, the contents of which are each incorporated herein by reference in their entireties as related to the use of insect cells in viral production, insofar as they do not conflict with the present disclosure.

In one embodiment, the AAV particles are made using the methods described in WO2015/191508, the content of which is incorporated herein by reference in its entirety, insofar as it does not conflict with the present disclosure.

In certain embodiments, insect host cell systems, in combination with baculoviral systems (e.g., as described by Luckow et al., Bio/Technology 6: 47 (1988)) may be used. In certain embodiments, an expression system for preparing chimeric peptide is Trichoplusia ni, Tn 5B1-4 insect cells/ baculoviral system, which can be used for high levels of proteins, as described in U.S. Pat. No. 6,660,521, the content of which is incorporated herein by reference in its entirety, insofar as it does not conflict with the present disclosure.

Expansion, culturing, transfection, infection and storage of insect cells can be carried out in any cell culture media, cell transfection media or storage media known in the art, comprising Hyclone SFX Insect Cell Culture Media, Expression System ESF AF Insect Cell Culture Mediwn, Basal IPL-41 Insect Cell Culture Media, ThermoFisher Sf90011 media, ThermoFisher Sf900III media, or ThermoFisher Grace's Insect Media. Insect cell mixtures of the present disclosure can also comprise any of the formulation additives or elements described in the present disclosure, comprising (but not limited to) salts, acids, bases, buffers, surfactants (such as Poloxatner 188/Pluronic. F-68), and other known culture media elements. Formulation additives can be incorporated gradually or as “spikes” (incorporation of large volumes in a short time).

### Baculovirus-Production Systems

In certain embodiments, processes of the present disclosure can comprise production of AAV particles or viral vectors in a baculoviral system using a viral expression construct and a payload construct vector. In certain embodiments, the baculoviral system comprises Baculovirus expression vectors (BEVs) and/or baculovirus infected insect cells (BIICs). In certain embodiments, a viral expression construct or a payload construct of the present disclosure can be a bacmid, also known as a baculovirus plasmid or recombinant baculovirus genome. In certain embodiments, a viral expression construct or a payload construct of the present disclosure can be polynucleotide incorporated by homologous recombination (transposon donor/acceptor system) into a bacmid by standard molecular biology techniques known and performed by a person skilled in the art. Transfection of separate viral replication cell populations produces two or more groups (e.g. two, three) of baculoviruses (BEVs), one or more group which can comprise the viral expression construct (Expression BEV), and one or more group which can comprise the payload construct (Payload BEV). The baculoviruses may be used to infect a viral production cell for production of AAV particles or viral vector.

In certain embodiments, the process comprises transfection of a single viral replication cell population to produce a single baculovirus (BEV) group which comprises both the viral expression construct and the payload construct. These baculoviruses may be used to infect a viral production cell for production of AAV particles or viral vector.

In certain embodiments, BEVs are produced using a Bacmid Transfection agent, such as Promega FuGENE HD, WFI water, or Thermaisher Cellfectin II Reagent. In certain embodiments, BEVs are produced and expanded in viral production cells, such as an insect cell.

In certain embodiments, the method utilizes seed cultures of viral production cells that comprise one or more BEVs, comprising baculovirus infected insect cells (BIICs). The seed BIICs have been transfected/transduced/infected with an Expression BEV which comprises a viral expression construct, and also a Payload BEV which comprises a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN₂ vapor.

Baculoviruses are made of several essential proteins which are essential for the function and replication of the Baculovirus, such as replication proteins, envelope proteins and capsid proteins, The Baculovirus genome thus comprises several essential-gene nucleotide sequences encoding the essential proteins. As a non-limiting example, the genome can comprise an essential-gene region which comprises an essential-gene nucleotide sequence encoding an essential protein for the Baculovirus construct. The essential protein can comprise: GP64 baculovirus envelope protein, VP39 baculovirus capsid protein, or other similar essential proteins for the Baculovirus construct.

Baculovirus expression vectors (BEV) for producing AAV particles in insect cells, comprising but not limited to Spodoptera frugiperda (SD) cells, provide high titers of viral vector product. Recombinant baculovirus encoding the viral expression construct and payload. construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the content of which is incorporated herein by reference in its entirety as related to the production and use of BEVs and viral particles, insofar as it does not conflict with the present disclosure.

Production of AAV particles with baculovirus in an insect cell system may address known baculovirus genetic and physical instability.

In certain embodiments, the production system of the present disclosure addresses baculovirus instability over multiple passages by utilizing a titerless infected-cells preservation and scale-up system. Small scale seed cultures of viral producing cells are ⁻transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture Wasilko D J et al. Protein Expr Purif. 2009 June; 65(2):122-32, the content of which is incorporated herein by reference in its entirety as related to the production and use of BEVs and viral particles, insofar as it does not conflict with the present disclosure.

A genetically stable baculovirus may be used to produce a source of the one or more of the components for producing AAV particles in invertebrate cells. In certain embodiments, defective baculovirus expression vectors may be maintained episomally in insect cells. In such an embodiment the corresponding bacmid vector is engineered with replication control elements, comprising but not limited to promoters, enhancers, and/or cell-cycle regulated replication elements.

In certain embodiments, baculoviruses may be engineered with a marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In certain embodiments, stable viral producing cells permissive for baculovirus infection are engineered with at least one stable integrated copy of any of the elements necessary for AAV replication and vector production comprising, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In certain embodiments, the Baculovirus expression vectors (BEV) are based on the AcMNPV baculovirus or BmNPV baculovirus BmNPV. In certain embodiments, a bacmid of the present disclosure is based on (i.e. engineered variant of) an AcMNPV bacmid such as bmon14272, vAce25ko or vAclef11KO.

In certain embodiments, the Baculovirus expression vectors (BEV) is a BEV in which the baculoviral v-cat/i gene has been partially or fully deleted (“v-cath deleted BEV”) or mutated. In certain embodiments, the BEVs lack the v-cath gene or comprise a mutationally inactivated version of the v-cath gene. In certain embodiments, the BEVs lack the v-cath gene. In certain embodiments, the BEVs comprise a mutationally inactivated version of the v-cath gene.

Viral production bacmids of the present disclosure can comprise deletion of certain baculoviral genes or loci.

The baculovirus/Sf9 system is a cGMP compatible and scalable manufacturing platform established for rAAV production. Baculoviral inoculum is a critical raw material for this manufacturing process and can have a significant impact on process yields and product quality. However, as with any other biological-based production processes, there is a large potential for batch-to-batch variability in the production of baculoviral inoculum. Each hank baculoviral inoculum requires extensive analytical characterization and optimized bioreactor production parameters to be used optimally in rAAV production (which adds time and cost for large-scale manufacturing). It is therefore advantageous to produce large banks of baculoviral inoculum, as these large banks allow for consistent and repeatable production of rAAV in large-scale bioreactors across multiple manufacturing campaigns from the same baculoviral inoculum bank.

In certain embodiments, baculoviral inoculum banks can be produced using small-scale shake flasks, such as 3L or 5L shake flasks. However, this process is generally limited in the maximum cell density of the BIIC cells which can be produced, and thus requires centrifugation to concentrate resulting cells into a workable concentration. This correspondingly limits the volume (i.e. quantity) of the baculoviral inoculum bank (˜600 mL) which can be produced and stored using this method. This process also presents sterility concerns due to open operation.

In certain embodiments, baculoviral inoculum banks can be produced using bioreactors, such as 20-50 L bioreactors. However, this process is also generally limited in the maximum cell density of the BIIC cells which can be produced, and thus requires significant processing through Tangential Flow Filtration (TFF) and/or centrifugation to concentrate resulting cells into a workable concentration (with 3 L of culture material being required to produce about 600 mL of concentrated BIIC formulation, corresponding with a 15-25% yield). This correspondingly limits the volume (i.e. quantity) of the baculoviral inoculum bank (˜3000 mL) which can be produced and stored using this method. This process also presents sterility concerns due to open operation.

In certain embodiments, perfusion technology can be used in the production of baculoviral inoculum banks. Perfusion systems are fluid circulation systems which use combinations of pumps, filters and screens to retain cells inside a bioreactor while continually removing cell waste products and replacing media depleted of nutrients by cell metabolism. In certain embodiments, the perfusion system is an alternating tangential flow (ATF) perfusion system. in certain embodiments, a perfusion system can be used in coordination with bioreactors to manage and cycle cell culture media within a bioreactor during the production of Baculovirus Infected Insect Cells (BACs). In certain embodiments, a perfusion system can be used to support the production of high quality BIIC banks having an unexpectedly high cell density at large-scale. In certain embodiments, a perfusion system can be used to provide an infection-cell-to-product-cell yield of greater than 70% (e.g. 75-80%, 80-85%, 85-90%, 90-95% or 95-100%). In certain embodiments, a perfusion system can be used to perform a media switch within the bioreactor, such as the replacements of a cell culture media with a cryopreservation media which allows for BIIC cells to be frozen and preserved.

The present disclosure presents methods for producing a baculovirus infected insect cell (BIIC). In certain embodiments, the present disclosure presents methods for producing a baculovirus infected insect cell (BIIC) which comprises the following steps: (a) introducing a volume of cell culture medium into a bioreactor; (b) introducing at least one viral production cell (VPC) into the bioreactor and expanding the number of VPCs in the bioreactor to a target VPC cell density; (c) introduction at least one Baculoviral Expression Vector (BEV) into the bioreactor, wherein the BEV comprises an AAV viral expression construct or an AAV payload construct; (d) incubating the mixture of VPCs and BEVs in the bioreactor under conditions which allow at least one BEV to infect at least one NIPC to produce a baculoviras infected insect cell (BIIC); (e) incubating the bioreactor under conditions which allow the number of BIICs in the bioreactor to reach a target BIIC cell density; and (f) harvesting the BIICs from the bioreactor. In certain embodiments, the bioreactor has a volume of at least 5 L, 10 L, 20 L, 50 L, 100 L, or 200 L. In certain embodiments, the volume of cell culture medium (i.e. working volume) in the bioreactor is at least 5 L, 10 L, 20 L, 50 L, 100 L, or 200 L.

In certain embodiments, the NIPC density at BEV introduction is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at BEV introduction is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, the target VPC cell density at BEV introduction is 1.5-4.0×10⁶ cells/mL. In certain embodiments, the target VPC cell density at BEV introduction is 2.0-3.5×10⁶ cells/mL.

In certain embodiments, the BE\/s are introduced into the bioreactor at a target Multiplicity of Infection (M01) of BEVs to VPCs. In certain embodiments, the BEV MIN is 0.0005-0.003, or more specifically 0.001-0.002.

In certain embodiments, the bioreactor can comprise a perfusion :stem for managing the cell culture medium within the bioreactor. in certain embodiments, the perfusion system is an alternating tangential flow (ATF) perfusion system. In certain embodiments, the perfusion system replaces at least a portion of the culture medium in the bioreactor while retaining at least 90% of the VPCs and FMCS within the bioreactor. In certain embodiments, the perfusion system removes cell waste products from the cell culture medium within the bioreactor. In certain embodiments, the perfusion system replaces cell culture media which has been depleted of nutrients by cellular metabolism. In certain embodiments, the perfusion system replaces the cell culture media with a cryopreservation media which allows for BIIC cells to be frozen and preserved. In certain embodiments, the perfusion system replaces the cell culture media with a cell culture media supplemented with growth or production boosting factors to increase the quality and quantity of the AAV product.

In certain embodiments, the BIICs are harvested from the bioreactor at a specific BIIC cell density. In certain embodiments, the BIICs harvested from the bioreactor have a specific BIIC cell density. In certain embodiments, the BIIC cell density at harvesting is 6.0-18.0×10⁶ cells/mL, 8.0-16.5×10⁶ cells/mL, 10.0-16.5×cells/mL.

### Other

In certain embodiments expression hosts comprise, but are not limited to, bacterial species within the genera Escherichia, Bacillus, Pseudomonas, or Salmonella.

In certain embodiments, a host cell which comprises AAV rep and cap genes stably integrated within the cell's chromosomes, may be used for AAV particle production. In a non-limiting example, a host cell which has stably integrated in its chromosome at least two copies of an AAV rep gene and AAV cap gene may he used to produce the AAV particle according to the methods and constructs described in U.S. Pat. No. 7,238,526, the content of which is incorporated herein by reference in its entirety as related to the production of viral particles, insofar as it does not conflict with the present disclosure.

In certain embodiments, the AAV particle can be produced in a host cell stably transformed with a molecule comprising the nucleic acid sequences which permit the regulated expression of a rare restriction enzyme in the host cell, as described in U.S. Pat. No. 20030092161. and EP1183380, the contents of which are each incorporated herein by reference in their entireties as related to the production of viral particles, insofar as they do not conflict with the present disclosure.

In certain embodiments, production methods and cell lines to produce the AAV particle may comprise, but are not limited to those taught in PCT/US1996/010245, PCT/US1997/015716, PCT/US1997/015691, PCT/US1998/019479, PCT/US1998/019463, PCT/US2000/000415, PCT/US2000/040872, PCT/US2004/01.6614, PCT/US2007/010055, PCT/US1999/005870, PCT/US2000/004755, US Patent Application Nos. US08/549489, US08/462014, US09/659203, US10/246447, US10/465302, U.S. Pat. Nos. 6,281,010, 6,270,996, 6,261,551, 5,756,283 (Assigned to NIH), U.S. Pat. Nos. 6,428,988, 6,274,354, 6,943,019, 6,482,634, (Assigned to NUM U.S. Pat. Nos. 7,238,526, 6,475,769), U.S. Pat. No. 6,365,394 (Assigned to NIH), U.S. Pat. Nos. 7,491,508, 7,291,498, 7,022,519, 6,485,966, 6,953,690, 6,258,595, EP2018421, EP1064393, EP1163354, EP835321, EP931158, EP950111, EP1015619, EP1183380, EP2018421, EP1226264, EP1636370, EP1163354, EP1064393, US20030032613, US20020102714, US20030073232, US20030040101 (Assigned to US20060003451, US20020090717, US20030092161, US20070231303, US20060211115, US20090275107, US2007004042, US20030119191, US20020019050, the contents of which are each incorporated herein by reference in their nireties, insofar as they do not conflict with the present disclosure.

### Viral Production Systems

### Large-Scale Production

In certain embodiments, AAV particle production may be modified to increase the scale of production. Large scale viral production methods according to the present disclosure may comprise any of the processes or processing steps taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference by reference in their entirety.

Methods of increasing AAV particle production scale typically comprise increasing the number of viral production cells. In certain embodiments, viral production cells comprise adherent cells. To increase the scale of AAV particle production by adherent viral production cells, larger cell culture surfaces are required. In certain embodiments, large-scale production methods comprise the use of roller bottles to increase cell culture surfaces, Other cell culture substrates with increased surface areas are known in the art. Examples of additional adherent cell culture products with increased surface areas comprise, but are not limited to iCELLis (Pall Corp, Port Washington, N.Y.), CELLSTACK®, CULCUBE® (Corning Corp., Corning, N.Y.) and NUNC™ CELL FACTORY™ (Thermo Scientific, Waltham, Mass.) In certain embodiments, large-scale adherent cell surfaces may comprise from about 1,000 cm² to about 100,000 cm².

In certain embodiments, large-scale viral production methods of the present disclosure may comprise the use of suspension cell cultures. Suspension cell culture can allow for significantly increased numbers of cells. Typically, the number of adherent cells that can be grown on about 10-50 cm² of surface area can be grown in about 1 cm³ volume in suspension.

In certain embodiments, large-scale cell cultures may comprise from about 10⁷ to about 10⁹ cells, from about 10⁸ to about 10¹⁰ cells, from about 10⁹ to about 10¹² cells or at least 10¹² cells. In certain embodiments, large-scale cultures may produce from about 10⁹ to about 10¹², from about 10¹⁶ to about 10¹³, from about 10¹¹ to about 10¹⁴, from about 10¹² to about 10¹⁵ or at least 10¹⁵ AAV particles.

Transfection of replication cells in large-scale culture formats may be carried out according to any methods known in the art. For large-scale adherent cell cultures, transfection methods may comprise, but are not limited to the use of inorganic compounds (e.g. calcium phosphate,) organic compounds (e.g. polyethyleneimine (PEI)) or the use of non-chemical methods (e.g. electroporation). With cells grown in suspension, transfection methods may comprise, but are not limited to the use of inorganic compounds (e.g., calcium phosphate,) organic compounds (e.g. polyethyleneimine (PEI)) or the use of non-chemical methods (e.g. electroporation). In certain embodiments, transfection of large-scale suspension cultures may be carried out according to the section entitled “Transfection Procedure” described in Feng, L. et al,, 2008. Biotechnol Appl Biochem. 50:121-32, the contents of which are herein incorporated by reference in their entirety. According to such embodiments. PEI-DNA complexes may be formed for introduction of plasmids to be transfected. In certain embodiments, cells being transfected with PEI-DNA complexes may be ‘shocked’ prior to transfection. This comprises lowering cell culture temperatures to 4° C. for a period of about I hour. In certain embodiments, cell cultures may be shocked for a period of from about 10 minutes to about 5 hours. In certain embodiments, cell cultures may be shocked at a temperature of from about 0° C. to about 20° C.

In certain embodiments, transfections may comprise one or more vectors for expression of an RNA effector molecule to reduce expression of nucleic acids from one or more payload construct. Such methods may enhance the production of AAV particles by reducing cellular resources wasted on expressing payload constructs. In certain embodiments, such methods may be carried according to those taught in US Publication No. US2014/0099666, the contents of which are herein incorporated by reference in their entirety.

### Bioreactors

In certain embodiments, cell culture bioreactors may be used for large scale production of AAV particles. In certain embodiments, bioreactors comprise stirred tank reactors. Such reactors generally comprise a vessel, typically cylindrical in shape, with a stirrer impeller.) in certain embodiments, such bioreactor vessels may be placed within a water jacket to control vessel temperature and/or to minimize effects from ambient temperature changes.

Bioreactor vessel volume may range in size from about 500 ml to about 2 L, from about 1 L to about 5 L, from about 2.5 L to about 20 L, from about 10 L to about 50 L, from about 25 L to about 100 L, from about 75 L to about 500 L, from about 250 L to about 2,000 L, from about 1,000 L to about 10,000 L, from about 5,000 L to about 50,000 L or at least 50,000 L. Vessel bottoms may be rounded or flat. In certain embodiments, animal cell cultures may be maintained in bioreactors with rounded vessel bottoms.

In certain embodiments, bioreactor vessels may be warmed through the use of a thermocirculator. Thermocirculators pump heated water around water jackets. In certain embodiments, heated water may be pumped through pipes (e.g. coiled pipes) that are present within bioreactor vessels. In certain embodiments, warm air may be circulated around bioreactors, comprising, but not limited to air space directly above culture medium. Additionally, pH and CO₂ levels may be maintained to optimize cell viability.

In certain embodiments, bioreactors may comprise hollow-fiber reactors. Hollow-fiber bioreactors may support the culture of both anchorage dependent and anchorage independent cells. Further bioreactors may comprise, but are not limited to, packed-bed or fixed-bed bioreactors. Such bioreactors may comprise vessels with glass beads for adherent cell attachment. Further packed-bed reactors may comprise ceramic beads.

In certain embodiments, viral particles are produced through the use of a disposable bioreactor. In certain embodiments, bioreactors may comprise GE WAVE bioreactor, a GE Xcellerax Bioreactor, a Sartorius Biostat Bioreactor, a ThermoFisher Hyclone Bioreactor, or a Pall Allegro Bioreactor.

In certain embodiments, AAV particle production in cell bioreactor cultures may be carried out according to the methods or systems taught in U.S. Pat. Nos. 5,064764, 6,194,191, 6,566,118, 8,137,948 or US Patent Application No. US2011/0229971, the contents of each of which are herein incorporated by reference in their entirety.

In certain embodiments, perfusion technology can be used in the production of viral particles. Perfusion systems are fluid circulation systems which use filters and screens to retain cells inside a bioreactor while continually removing cell waste products and media depleted of nutrients by cell metabolism. In certain embodiments, the perfusion system is an alternating tangential flow (ATF) perfusion system. In certain embodiments, a perfusion system can be used in coordination with bioreactors to manage and cycle cell culture media within a bioreactor during the production of viral particles, such as AAV viral particles. In certain embodiments, a perfusion system can be used to support the production of high quality AAV viral particles having an unexpectedly high cell density at large-scale. In certain embodiments, a perfusion system can be used to perform a media switch within the bioreactor, such as the replacement of a cell culture media with media supplemented with growth or production boosting; factors to increase the quality and quantity of the AAV product.

It is advantageous to produce large batches of AAV particles in single production campaigns for gene therapy clinical development activities, as the large batches of therapeutic materials ensure clinical study consistency and minimize the therapeutic and statistical variability resulting from multiple smaller manufacturing campaigns. It is advantageous to produce large batches of AAV particles in single production campaigns for commercial product development activities, as the large batches of therapeutic materials minimize the variability resulting from multiple smaller manufacturing campaigns and corresponding complications in quality control and product analysis associated with small-batch production.

### Expansion of Viral Production Cell (VPC) Mixtures

In certain embodiments, an AAV particle or viral vector of the present disclosure may be produced in a viral production cell (VPC), such as an insect cell. Production cells can be sourced from a Cell Bank (CB) and are often stored in frozen cell banks.

In certain embodiments, a viral production cell from a Cell Bank is provided in frozen form. The vial of frozen cells is thawed, typically until ice crystal dissipate. In certain embodiments, the frozen cells are thawed at a temperature between 10-50° C., 15-40° C., 20-30° C., 25-50° C., 30-45° C., 35-40° C., or 37-39° C. In certain embodiments, the frozen viral production cells are thawed using a heated water bath.

In certain embodiments, a thawed CB cell mixture will have a cell density of 1.0×10⁴-1.0×10⁹ cells/mL. In certain embodiments, the thawed CB cell mixture has a cell density of 1.0×10⁴-2.5×10⁴ cells/mL, 2.5×10⁴-5.0×10⁴ cells/int, 5.0×10⁴-7.5×10⁴ cells/mL, 7.5×10⁴-1.0×10⁵ cell s/mL, 1.0×10⁵-2.5×10⁵ cells/mL, 2.5×10⁵-5.0×10⁵ cells/int, 5.0×10⁵-7.5×10⁵ cells/mL. 7.5×10⁵-1.0×10⁶ cells/mL, 1.0×10⁶-2.5×10⁶ cells/mL, 2.5×10⁶-5.0×10⁶ cells/mL, 5.0×10⁶-7.5×10⁶ cells/int, 7.5×10⁶-1.0×10⁷ cells/mL, 1.0×10⁷-2.5×10⁷ cells/mL, 2.5×10⁷-5.0×10⁷ cells/mL, 5.0×10⁷-7.5×10⁷ cells/mL, 7.5×10⁷-1.0×10⁸ 1.0×10⁸-2.5×10⁸ cells/mL, 2.5×10⁸-5.0×10⁸ cells/mL, 5.0×10⁸-7.5×10⁸ cells/mL, or 7.5×10⁸-1.0×10⁹ cells/mL.

In certain embodiments, the volume of the CB cell mixture is expanded. This process is commonly referred to as a Seed Train, Seed Expansion, or CB Cellular Expansion. Cellular/Seed expansion can comprise successive steps of seeding and expanding a cell mixture through multiple expansion steps using successively larger working volumes. In certain embodiments, cellular expansion can comprise one, two, three, four, five, six, seven, or more than seven expansion steps. In certain embodiments, the working volume in the cellular expansion can comprise one or more of the following working volumes or working volume ranges: 5 mL, 10 mL, 20 mL, 5-20 mL, 25 mL, 30 mL, 40 mL, 50 mL, 20-50 mL, 75 mL, 100 mL, 125 mL, 150 mL, 175 mL, 200 mL, 50-200 mL, 250 mL, 300 mL, 400 mL, 500 mL, 750 mL, 1000 mL, 250-1000 mL, 1250 mL, 1500 mL, 1750 mL, 2000 mL, 1000-2000 mL, 2250 mL, 2500 mL, 2750 mL, 3000 mL, 2000-3000 mL, 3500 mL, 4000 mL, 4500 mL, 5000 mL, 3000-5000 mL, 5.5 L, 6.0 L, 7.0 L, 8.0 L, 9.0 L. 10.0 L, and 5.0-10.0 L.

In certain embodiments, a volume of cells from a first expanded cell mixture can be used to seed a second, separate Seed Train/Seed Expansion (instead of using thawed CB cell mixture). This process is commonly referred to as rolling inoculum. In certain embodiments, rolling inoculum is used in a series of two or more (e.g. two, three, four or five)separate Seed Trains/Seed Expansions.

In certain embodiments, large-volume cellular expansion can comprise the use of a bioreactor, such as a GE WAVE bioreactor, a GE Xcellerax Bioreactor, a Sartorius Biostat Bioreactor, a ThermoFisher Hyclone Bioreactor, or a Pall Allegro Bioreactor.

In certain embodiments, the cell density within a working volume is expanded to a target output cell density. In certain embodiments, the output cell density of an expansion step is 1.0×10⁵-5.0×10⁵, 5.0×10⁵-1.0×1.0⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10.0×10⁷, 4.0×10⁷, 5.0×10⁷, 6.0×10⁷, 7.0×10⁷, 8.0×10⁷, or 9.0×10⁷ cells/mL.

In certain embodiments, the output cell density of a working volume provides a seeding cell density for a larger, successive working volume. In certain embodiments, the seeding cell density of an expansion step is 1.0×10⁵-5.0×10⁵, 5.0×10⁵-10×10⁶, 1.0×10⁶-5.0×10⁶, 5.0×10⁶-1.0×10⁷, 1.0×10⁷-5.0×10⁷, 5.0×10⁷-1.0×10⁸, 5.0×10¹¹ , 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, cellular expansion can last for 1-50 days. Each cellular expansion step or the total cellular expansion can last for 1-10 days, 1-5 days, 1-3 days, 2-3 days, 2-4 days, 2-5 days, 2-6 days, 3-4 days, 3-5 days, 3-6 days, 3-8 days, 4-5 days, 4-6 days, 4-8 days, 5-6 days, or 5-8 days. In certain embodiments, each cellular expansion step or the total cellular expansion can last for 1-100 generations, 1-1000 generations, 100-1000 generation, 100 generations or more, or 1000 generation or more.

In certain embodiments, infected or transfected production cells can be expanded in the same manner as CB cell mixtures, as set forth in the present disclosure.

### Infection of Viral Production Cells

In certain embodiments. AAV particles of the present disclosure are produced in a viral production cell (VPC), such as an insect cell, by infecting the VPC with a viral vector which comprises an AAV expression construct and/or a viral vector which comprises an AAV payload construct. In certain embodiments, the VPC is infected with an Expression BEV, which comprises an AAV expression construct and a Payload BEV which comprises an AAV payload construct.

In certain embodiments, AAV particles are produced by infecting a VPC with a viral vector which comprises both an AAV expression construct and an AAV payload construct. In certain embodiments, the VPC is infected with a single BEV which comprises both an AAV expression construct and an AAV payload construct.

In certain embodiments, VPCs (such as insect cells) are infected using Infection BIICs in an infection process which comprises the following steps: (i) A collection of VPCs are seeded into a Production Bioreactor; (ii) The seeded VPCs can optionally be expanded to a target working volume and cell density; (iii) Infection BIICs which comprise Expression BEVs and Infection BIICs which comprise Payload BEVs are injected into the Production Bioreactor, resulting in infected viral production cells; and (iv) incubation of the infected viral production cells to produce AAV particles within the viral production cells.

In certain embodiments, the VPC density at infection is 1.0×10⁵-2.5×10⁵, 2.5×10⁵-5.0×10⁵, 5.0×1.0⁵-7.5×10⁵, 7.5×1.0⁵-1.0×10⁶, 1.0×1.0⁶-5.0×10⁶, 1.0×1.0⁶-2.0×10⁶, 2.5×10⁶, 2.0×10⁶-3.0×10⁶, 2.5×10⁶-3.5×10⁶, 3.0×10⁶-3.4×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×1.0⁷, 9.5×10° -1.5×1.0⁷, 1.0×10⁷-5.0×10⁷, or 5.0×10⁷-1.0×10⁸ cells/mL. In certain embodiments, the VPC density at infection is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.1×10⁶, 3.2×10⁶, 3.3×10⁶, 3.4×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL. In certain embodiments, the VPC density at infection is 2.0-3.5×10⁶ cells/mt. In certain embodiments, the VPC density at infection is 3.5-5.0×10⁶ cells/mL. In certain embodiments, the VPC density at infection is 5.0-7.5×10⁶ cells/mL. In certain embodiments, the VPC density at infection is 5.0-10.0×10⁶ cells/mL.

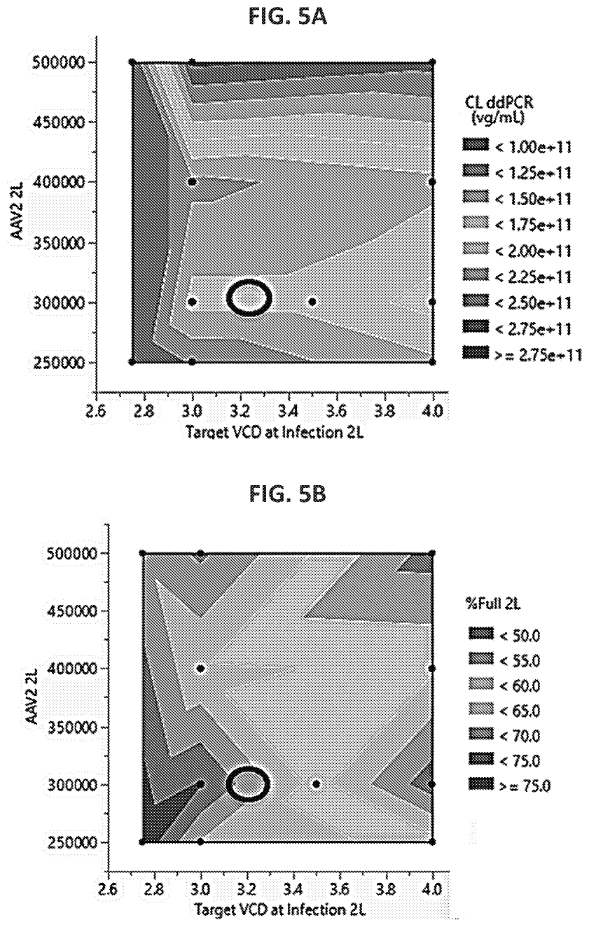

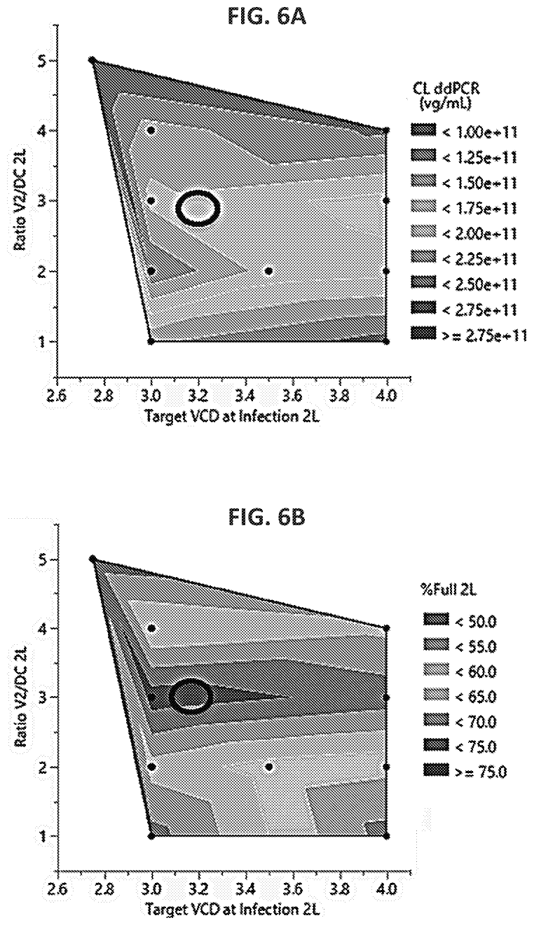

FIG. 5A, FIG. 5B, FIG. 6A and. FIG. 6B show that a VPC density at transfection/infection of between 3.0-3.4×10⁶ cells/mL, particularly 3.2×10⁶ cells/mL, combined with a VPC-to-BIIC^{Rep/Cap} ratio of about 1:300K v/v, provides favorable AAV titer (vg/mL) and Capsid Full %.

In certain embodiments, Infection BIICs are combined with the VPCs in target ratios of VPC-to-BIIC. In certain embodiments, the VPC-to-BIIC infection ratio (volume to volume) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁶, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ BIIC-per-VPC. In certain embodiments, the VPC-to-BIIC infection ratio (cell to cell) is 1.0×1.0³-5.0×10³, 5.0×10³-1.0×10⁴, 1.0×1.0⁴-5.0×10⁴, 5.0×10⁴-1.0×10⁵, 1.0×1.0⁵-5.0×10⁵, 5.0×10⁵-1.0×10⁶, 1.0×10³, 2.0×10³, 3.0×10³, 4.0×10³, 5.0×10³ 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ BIIC-per-VPC.

In certain embodiments, Infection BIICs which comprise Expression BEVs are combined with the VPCs in target ratios of VPC-to-BIIC. In certain embodiments, the VPC-to-BIIC infection ratio (volume to volume) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁵, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×1.0⁵, 7.0×10⁵, 8.0×10⁵, or 9.0×10⁵ BIIC-per-VPC. In certain embodiments, the VPC-to-BIIC infection ratio (cell to cell) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁶, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ BIIC-per-VPC.

In certain embodiments, Infection BIICs which comprise Payload BEVs are combined with the VPCs in target ratios of VPC-to-BHC, In certain embodiments, the NPC-to-BIIC infection ratio (volume to volume) is 1.0×10³-5.0×10³, 5.0×10³-1.0×10⁴, 1.0×10⁴-5.0×10⁴, 5.0×10⁴-1.0×10⁵, 1.0×10⁵-5.0×10⁵, 5.0×10⁵-1.0×10⁶, 1.0×10³, 2.0×10³, 3.0×10³, 4.0×10³, 5.0×10³, 6.0×10³, 7.0×10³, 8.0×10³, 9.0×10³, 1.0×10⁴, 2.0×10⁴, 3.0×10⁴, 4.0×10⁴, 5.0×10⁴, 6.0×10⁴, 7.0×10⁴, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ BHC-per-VPC. In certain embodiments, the VPC-to-BIIC infection ratio (cell to cell) is 1.0×10³-5.0×10³, 5.0×10³-1.0×10⁴, 1.0×10⁴-5.0×10⁴, 5.0×1.0⁴-1.0×10⁵, 1.0×1.0⁵-5.0×10⁵, 5.0×10⁵-1.0×10⁶, 1.0×1.0³, 2.0×10³, 3.0×10³, 4.0×10³, 5.0×10³, 6.0×10³, 7.0×10³, 8.0×10³, 9.0×10³, 10×10⁴, 2.0×10⁴, 3.0×10⁴, 4.0×10⁴, 5.0×10⁴, 6.0×10⁴, 7.0×10⁵, 8.0×10⁴, or 9.0×10⁴, 1.0×10⁵, 2.0×10⁵, 3.0×10⁵, 4.0×10⁵, 5.0×10⁵, 6.0×10⁵, 7.0×10⁵, 8.0×10⁵, or 9.0×10⁵ BIIC-per-VPC.

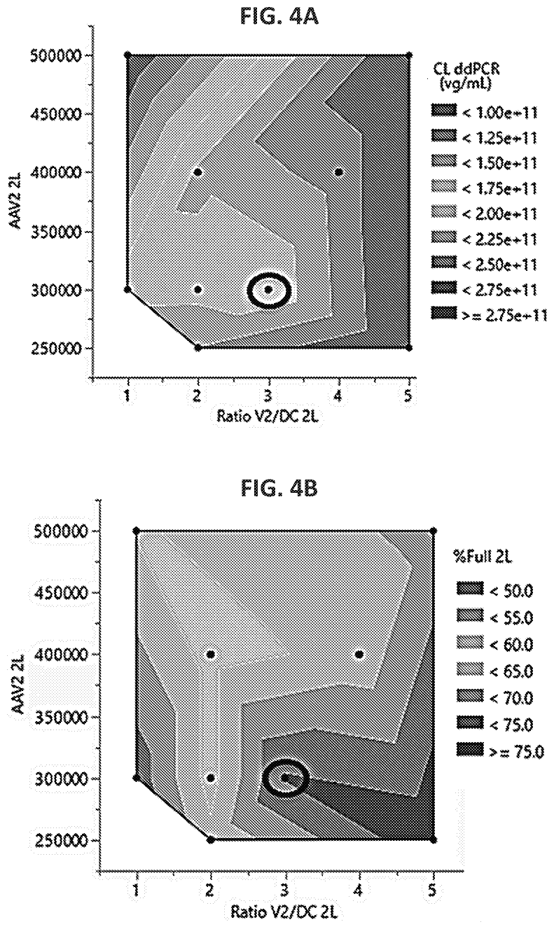

In certain embodiments, Infection BIICs which comprise Expression BEVs and Infection BIICs which comprise Payload BEVs are combined with the VPCs in target BIIC-to-BIIC ratios. In certain embodiments, the ratio of Expression (Rep/Cap) BIICs to Payload RUCs is 10:1, 9:1, 8:1, 7:1, 6:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4 1:4.5, 1:5, 1:5,5, 1:6, 1:6.5, 1:7, 1:7.5, 1:8, 1:9, 1:10, 3.5-4.5:1, 3-4:1, 2.5-3.5:1, 2-3:1, 1.5-2.5:1, 1-2:1, 1-1.5:1, 1:1-1.5, 1:1-2, 1:1.5-2.5, 1:2-3, 1:2.5-3.5, 1:3-4, 1:3.5-4.5, 1:4-5, 1:4.5-5.5, 1:5-6, 1:5.5-6.5, 1:6-7, or 1:6.5-7.5. 105601 FIG. 4A, FIG. 4B, FIG. 6A and FIG. 6B show that a VPC density at transfection/infection of between 3.0-3.4×10⁶ cells/mL, particularly 3.2×10⁶ cells/mL, combined with a VPC-to-BIIC^{Rep/Cap} ratio of about 1:300K v/v and a VPC-to-BIIC^{Payload} ratio of about 1:100K v/v (3:1 BIIC-to-BIIC ratio), provide favorable AAV titer (vg/mL) and Capsid Full %.

In certain embodiments, infected Viral Production Cells are incubated under a certain Dissolved Oxygen (DO) Content (DO %). In certain embodiments, infected Viral Production Cells are incubated under a DO % between 10%-50%, 20%-40%, 10%-20%, 15%-25%, 20%-30%, 25%-35%, 30%-40%, 35%-45%, 40%-50%, 10%-15%, 15%-20%, 20%-25%, 25%-30%, 30%-35%, 35%-40%, 40%-45%, or 45%-50%. In certain embodiments, infected Viral Production Cells are incubated under a DO % of about 10%, about 15%, about 20%, about 25%, about 30%, about 35%, about 40%, about 45%, about 50%. In certain embodiments, infected Viral Production Cells are incubated under a DO % between 20%-30% or about 25%. In certain embodiments, infected Viral Production Cells are incubated under a DO % between 25%-35% or about 30%. In certain embodiments, infected Viral Production Cells are incubated under a DO % between 30%-40% or about 35%. in certain embodiments, infected Viral Production Cells are incubated under a DO % between 35%-45% or about 40%.

### Cell Lysis

Cells of the present disclosure, comprising, but not limited to viral production cells, may he subjected to cell lysis according to any methods known in the art. Cell lysis may be carried out to obtain one or more agents (e.g. viral particles) present within any cells of the disclosure. In certain embodiments, a bulk harvest of AAV particles and viral production cells is subjected to cell lysis according to the present disclosure.

In certain embodiments, cell lysis may be carried out according to any of the methods or systems presented in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Cell lysis methods and systems may be chemical or mechanical. Chemical cell lysis typically comprises contacting one or more cells with one or more chemical lysis agent under chemical lysis conditions. Mechanical lysis typically comprises subjecting one or more cells to cell lysis carried out by mechanical force. Lysis can also be completed by allowing the cells to degrade after reaching ˜0% viability.

In certain embodiments, chemical lysis may be used to lyse cells. As used herein, the term “chemical lysis agent” refers to any agent that may aid in the disruption of a cell. In certain embodiments, lysis agents are introduced in solutions, termed lysis solutions or lysis buffers. As used herein, the term “chemical lysis solution” refers to a solution (typically aqueous) comprising one or more lysis agent. In addition to lysis agents, lysis solutions may comprise one or more buffering agents, solubilizing agents, surfactants, preservatives, cryoprotectants, enzymes, enzyme inhibitors and/or chelators. Lysis buffers are lysis solutions comprising one or more buffering agent. Additional components of lysis solutions may comprise one or more solubilizing agent. As used herein, the term “solubilizing agent” refers to a compound that enhances the solubility of one or more components of a solution and/or the solubility of one or more entities to which solutions are applied. In certain embodiments, solubilizing agents enhance protein solubility. In certain embodiments, solubilizing agents are selected based on their ability to enhance protein solubility while maintaining protein conformation and/or activity.

Exemplary lysis agents may comprise any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In certain embodiments, lysis agents may be selected from lysis salts, amphoteric agents, cationic agents, ionic detergents and non-ionic detergents. Lysis salts may comprise, but are not limited to, sodium chloride (NaCl) and potassium chloride (KCl) Further lysis salts may comprise any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety.

In certain embodiments, the cell lysate solution comprises a stabilizing additive. In certain embodiments, the stabilizing additive can comprise trehalose, glycine betaine, mannitol, potassium citrate, CuCl₂, proline, xylitol, NDSB 201, CTAB and K₂PO₄. In certain embodiments, the stabilizing additive can comprise amino acids such as arginine, or acidified amino acid mixtures such as arginine HCl. In certain embodiments, the stabilizing additive can comprise 0,1 M arginine or arginine HCl. In certain embodiments, the stabilizing additive can comprise 0.2 M arginine or arginine HCl. In certain embodiments, the stabilizing additive can comprise 0.25 M arginine or arginine HCl. In certain embodiments, the stabilizing additive can comprise 0.3 M arginine or arginine HCl. In certain embodiments, the stabilizing additive can comprise 0.4 M arginine or arginine HCl. In certain embodiments, the stabilizing additive can comprise 0.5 M arginine or arginine HCl. In certain embodiments, the stabilizing additive can comprise 0.6 M arginine or arginine HCl. In certain embodiments, the stabilizing additive can comprise 0.7 M arginine or arginine HCl. In certain embodiments, the stabilizing additive can comprise 0.8 M arginine or arginine HCl. In certain embodiments, the stabilizing additive can comprise 0.9 M arginine or arginine HCl. In certain embodiments, the stabilizing additive can comprise 1.0M arginine or arginine HCl.

Concentrations of salts may be increased or decreased to obtain an effective concentration for the rupture of cell membranes. Amphoteric agents, as referred to herein, are compounds capable of reacting as an acid or a base. Amphoteric agents may comprise, but are not limited to lysophosphatidylcholine, 3((3-Cholamidopropyl) dimethylammonium)-1-propanesulfonate (CHAPS), ZWITTERGENT® and the like. Cationic agents may comprise, but are not limited to, cetyltrimethylammonium bromide (C (16) TAB) and Benzalkonium chloride. Lysis agents comprising detergents may comprise ionic detergents or non-ionic detergents.

Detergents may function to break apart or dissolve cell structures comprising, but not limited to cell membranes, cell walls, lipids, carbohydrates, lipoproteins and glycoproteins. Exemplary ionic detergents comprise any of those taught in U.S. Pat. Nos. 7,625,570 and 6,593,123 or US Publication No. US2014/0087361, the contents of each of which are herein incorporated by reference in their entirety. In certain embodiments, the lysis solution comprises one or more ionic detergents. Example of ionic detergents for use in a lysis solution comprise, but are not limited to, sodium dodecyl sulfate (SDS), cholate and deoxycholate. In certain embodiments, ionic detergents may be comprised in lysis solutions as a solubilizing agent. In certain embodiments, the lysis solution comprises one or more nonionic detergents. Non-ionic detergents for use in a lysis solution may comprise, but are not limited to, octylgiucoside, digitonin, lubrol, C12E8, TWEEN®-20, TWEEN®-80, Triton X-100, Triton X-114, Brij-35, Brij-58, and Noniodet P-40. Non-ionic detergents are typically weaker lysis agents but may be comprised as solubilizing agents for solubilizing cellular and/or viral proteins. In certain embodiments, the lysis solution comprises one or more zwitterionic detergents. Zwitterionic detergents for use in a lysis solution may comprise, but are not limited to: Lauryl dimethylamine N-oxide (LDAO); N,N-Dimethyl-N-dodecylglycine betaine (Empigen® BB); 3-(N,N-Dimethylmyristylammonio)propanesulfonate (Zwittergent® 3-10); n-Dodecyl-N,N-dimethyl-3-ammonio-1-propariesulfonate (Zwittergent® 3-12); n-Tetradecyl-N,N-dimethyl-3-ammonio-1-propanesulfonate (Zwittergent® 3-14); 3-(1\,N-Dimethylpalmitylammonio) propanesulfonate (Zwittergent® 3-16); 3-((3-cholainidopropyl)dimethylammonio)-1-propariesulfonate (CHAPS); and 3-([3-Cholainidopropyl]dimethylammonio)-2-hydroxy-1-propanesulfonate (CHAPSO).

In certain embodiments, the lysis solution comprises Triton X-100, such as 0.5% w/v of Triton X-100. In certain embodiments, the lysis solution comprises Lauryldimethylamine N-oxide (LDAO), such as 0.184% w/v (4×CMC) of LDAO. In certain embodiments, the lysis solution comprises a seed oil surfactant such as Ecosurf™ SA-9. In certain embodiments, the lysis solution comprises N,N-Dimethyl-N-dodec,71₁,*,7cine betaine (Empigen® BB). In certain embodiments, the lysis solution comprises a Zwittergent® detergent, such as Zwittergent® 3-12 (n-Dodecyl-N,N-dimethyl-3-ammonio-1-propanesulfonate), Zwittergent® 3-14 (n-Tetradecyl-N,N-dimethyl-3-ammonio-1-propanesulfonate), or Zwittergent® 3-16 (3-(N,N-Dimethyl palmitylammonio)propanesulfonate).

Further lysis agents may comprise enzymes and urea. In certain embodiments, one or more lysis agents may be combined in a lysis solution in order to enhance one or more of cell lysis and protein solubility. In certain embodiments, enzyme inhibitors may be comprised in lysis solutions in order to prevent proteolysis that may be triggered by cell membrane disruption.

In certain embodiments, the lysis solution comprises between 0.1-1.0% w/v, between 0.2-0.8% w/v, between 0.3-0.7% w/v, between 0.4-0.6% w/v, or about 0.5% w/v of a cell lysis agent (e.g. detergent). In certain embodiments, the lysis solution comprises between 0.3-0.35% w/v, between 0.35-0.4% w/v, between 0.4-0.45% w/v, between 0.45-0.5% w/v, between 0.5-0.55% w/v, between 0.55-0.6% w/v, between 0.6-0.65% w/v, or between 0.65-0.7% w/v of a cell lysis agent (e.g. detergent).

In certain embodiments, cell lysates generated from adherent cell cultures may be treated with one more nuclease, such as Benzonase nuclease (Grade I, 99% pure) or c-LEcta. Denarase nuclease (formerly Sartorius Denarase). In certain embodiments, nuclease is added to lower the viscosity of the lysates caused by liberated DNA.

In certain embodiments, chemical lysis uses a single chemical lysis mixture. In certain embodiments, chemical lysis uses several lysis agents added in series to provide a final chemical lysis mixture.

In certain embodiments, a chemical lysis mixture comprises an acidified amino acid mixture (such as arginine HCl), a non-ionic detergent (such as Triton X-100), and a nuclease (such as Benzonase nuclease). In certain embodiments, the chemical lysis mixture can comprise an acid or base to provide a target lysis pH.

In certain embodiments, chemical lysis is conducted under chemical lysis conditions. As used herein, the term “chemical lysis conditions” refers to any combination of environmental conditions (e.g., temperature, pressure, pH, etc) which targets cells can be lysed by a chemical lysis agent.

In certain embodiments, the lysis pH is between 3.0-3.5, 3.5-4.0, 4.0-4.5, 4.5-5.0, 5.0-5.5, 5.5-6.0, 6.0-6.5, 6.5-7.0, 7.0-7.5, or 7.5-8.0. In certain embodiments, the lysis pH is between 6.0-7.0, 6.5-7.0, 6.5-7.5, or 7.0-7.5.

In certain embodiments, the lysis temperature is between 15-35° C. between 20-30° C., between 25-39° C., between 20-21° C., between 20-22° C., between 21-22° C., between 21-23° C., between 22-23° C., between 22-24° C., between 23-24° C., between 23-25° C., between 24-25° C., between 24-26° C., between 25-26° C., between 25-27° C., between 26-27° C., between 26-28° C., between 27-28° C., between 27-29 CC, between 28-29° C., between 28-30° C., between 29-30° C., between 29-31° C., between 30-31° C., between 30-32° C., between 31-32° C., or between 31-33° C,.

In certain embodiments, mechanical cell lysis is carried out. Mechanical cell lysis methods may comprise the use of one or more lysis condition and/or one or more lysis force. As used herein, the term “lysis condition” refers to a state or circumstance that promotes cellular disruption. Lysis conditions may comprise certain temperatures, pressures, osmotic purity, salinity and the like. In certain embodiments, lysis conditions comprise increased or decreased temperatures. According to certain embodiments, lysis conditions comprise changes in temperature to promote cellular disruption. Cell lysis carried out according to such embodiments may comprise freeze-thaw lysis. As used herein, the term “freeze-thaw lysis” refers to cellular lysis in which a cell solution is subjected to one or more freeze-thaw cycle. According to freeze-thaw lysis methods, cells in solution are frozen to induce a mechanical disruption of cellular membranes caused by the formation and expansion of ice crystals. Cell solutions used according freeze-thaw lysis methods, may further comprise one or more lysis agents, solubilizing agents, buffering agents, cryoprotectants, surfactants, preservatives, enzymes, enzyme inhibitors and/or chelators. Once cell solutions subjected to freezing are thawed, such components may enhance the recovery of desired cellular products. In certain embodiments, one or more cryoprotectants are comprised in cell solutions undergoing freeze-thaw lysis. As used herein, the term “cryoprotectant” refers to an agent used to protect one or more substance from damage due to freezing. Cryoprotectants may comprise any of those taught in US Publication No. US2013/0323302 or U.S. Pat. Nos. 6,503,888, 6,180,613, 7,888,096, 7,091,030, the contents of each of which are herein incorporated by reference in their entirety. In certain embodiments, cryoprotectants may comprise, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose and urea. In certain embodiments, freeze-thaw lysis may be carried out according to any of the methods described in U.S. Pat. No. 7,704,721, the contents of which are herein incorporated by reference in their entirety.

As used herein, the term “lysis force” refers to a physical activity used to disrupt a cell. Lysis forces may comprise, but are not limited to mechanical forces, sonic forces, gravitational forces, optical forces, electrical forces and the like. Cell lysis carried out by mechanical force is referred to herein as “mechanical lysis.” Mechanical forces that may be used according to mechanical lysis may comprise high shear fluid forces. According to such methods of mechanical lysis, a microfluidizer may he used. Microfluidizers typically comprise an inlet reservoir where cell solutions may be applied. Cell solutions may then be pumped into an interaction chamber via a pump (e.g. high-pressure pump) at high speed and/or pressure to produce shear fluid forces. Resulting lysates may then be collected in one or more output reservoir. Pump speed and/or pressure may he adjusted to modulate cell lysis and enhance recovery of products (e.g. viral particles.) Other mechanical lysis methods may comprise physical disruption of cells by scraping.

Cell lysis methods may be selected based on the cell culture format of cells to be lysed. For example, with adherent cell cultures, some chemical and mechanical lysis methods may be used. Such mechanical lysis methods may comprise freeze-thaw lysis or scraping. In another example, chemical lysis of adherent cell cultures may be carried out through incubation with lysis solutions comprising surfactant, such as Triton-X-100.

In certain embodiments, a method for harvesting AAV particles without lysis may be used for efficient and scalable AAV particle production. In a non-limiting example, AAV particles may be produced by culturing an AAV particle lacking a heparin binding site, thereby allowing the AAV particle to pass into the supernatant, in a cell culture, collecting supernatant from the culture; and isolating the AAV particle from the supernatant, as described in US Patent Application 20090275107, the contents of which are incorporated herein by reference in their entirety.

### Clarification and Purification: General

Cell lysates comprising viral particles may be subjected to clarification and purification. Clarification generally refers to the initial steps taken in the purification of viral particles from cell lysates and serves to prepare lysates for further purification by removing larger, insoluble debris from a bulk lysis harvest. Viral production can comprise clarification steps at any point in the viral production process. Clarification steps may comprise, but are not limited to, centrifugation and filtration. During clarification, centrifugation may be carried out at low speeds to remove larger debris only. Similarly, filtration may be carried out using filters with larger pore sizes so that only larger debris is removed.

Purification generally refers to the final steps taken in the purification and concentration of viral particles from cell lysates by removing smaller debris from a clarified lysis harvest in preparing a final Pooled Drug Substance. Viral production can comprise purification steps at any point in the viral production process. Purification steps may comprise, but are not limited to, filtration and chromatography. Filtration may be carried out using filters with smaller pore sizes to remove smaller debris from the product or with larger pore sizes to retain larger debris from the product. Filtration may be used to alter the concentration and/or contents of a viral production pool or stream. Chromatography may be carried out to selectively separate target particles from a pool of impurities.

Large-scale production of high-concentration AAV formulations is complicated by the tendency for high concentrations of AAV particles to aggregate or agglomerate. Small scale clarification and concentration systems, such as dialysis cassettes or spin centrifugation, are generally not sufficiently scalable for large-scale production. The present disclosure provides embodiments of a clarification, purification and concentration system for processing large volumes of high-concentration AAV production formulations. In certain embodiments, the large-volume clarification system comprises one or more of the following processing steps: Depth Filtration, Microfiltration (e.g. 0.2 μm Filtration). Affinity Chromatography, Ion Exchange Chromatography such as anion exchange chromatography (AEX) or cation exchange chromatography (CEX), a tangential flow filtration system (TFF), Nanofiltration (e.g. Virus Retentive Filtration (VIM), Final Filtration (FT), and Fill Filtration,

Objectives of viral clarification and purification comprise high throughput processing of cell lysates and to optimize ultimate viral recovery. Advantages of comprising clarification and purification steps of the present disclosure comprise scalability for processing of larger volumes of lysate, In certain embodiments, clarification and purification may be carried out according to any of the methods or systems presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos, US2013/0045186, US2011/0263027, US2011/0151434, US200310138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

In certain embodiments, the compositions comprising at least one AAV particle may be isolated or purified using the methods or systems described in U.S. Pat. Nos. 6,146,874, 6,660,514, 8,283,151 or 8,524,446, the contents of which are herein incorporated by reference in their entirety.

### Clarification and Purification: Centrifugation

According to certain embodiments, cell lysates may be clarified by one or more centrifugation steps. Centrifugation may be used to pellet insoluble particles in the lysate. During clarification, centrifugation strength (which can be expressed in terms of gravitational units (g), which represents multiples of standard gravitational force) may be lower than in subsequent purification steps. In certain embodiments, centrifugation may be carried out on cell lysates at a gravitation force from about 200 g to about 800 g, from about 500 g to about 1500 g, from about 1000 g to about 5000 g, from about 1200 g to about 10000 g or from about 8000 g to about 15000 g. In certain embodiments, cell lysate centrifugation is carried out at 8000 g for 15 minutes. In certain embodiments, density gradient centrifugation may be carried out in order to partition particulates in the cell lysate by sedimentation rate. Gradients used according to methods or systems of the present disclosure may comprise, but are not limited to, cesium chloride gradients and iodixanol step gradients. In certain embodiments, centrifugation uses a decanter centrifuge system. In certain embodiments, centrifugation uses a disc-stack centrifuge system. In certain embodiments, centrifugation comprises ultracentrifugation, such two-cycle CsCl gradient ultracentrifugation or iodixanol discontinuous density gradient ultracentrifugation.

### Clarification and Purification: Filtration

In certain embodiments, one or more microfiltration, nanofiltration and/or ultrafiltration steps may be used during clarification, purification and/or sterilization. The one or more microfiltration, nanofiltration or ultrafiltration steps can comprise the use of a filtration system such as EMD Millipore Express SHC XTIO 0.5/0.2 μm filter, EMD Millipore Express SFICXL6000 0.5/0.2 μm filter, EMD Millipore Express SFICXL150 filter, EMD Millipore Millipak Gamma Gold 0.22 μm filter (dual-in-line sterilizing grade filters), a Pall Supor EKV, 0.2 μm sterilizing-grade filter, Asahi Planova 35N, Asahi Planova 20N, Asahi Planova 75N, Asahi Planova BioEx, Millipore Viresolve NFR or a Sartorius Sartopore 2XLG, 0.8/0.2 μm.

In certain embodiments, one or more microfiltration steps may be used during clarification, purification and/or sterilization. Microfiltration utilizes microfiltration membranes with pore sizes typically between 0.1 μm and 10 μm. Microfiltration is generally used for general clarification, sterilization, and removal of microparticulates. In certain embodiments, microfiltration is used to remove aggregated clumps of viral particles. In certain embodiments, a production process or system of the present disclosure comprises at least one microfiltration step. The one or more microfiltration steps can comprise a Depth Filtration step with a Depth Filtration system, such as EMD Millipore Millistak⁺ POD filter (D0HC media series), Millipore MC0SP23CL3 filter (COSP media series), or Sartorius Sartopore filter series. Microfiltration systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, one or more ultrafiltration steps may be used during clarification and purification. The ultrafiltration steps can be used for concentrating, formulating, desalting or dehydrating either processing and/or formulation solutions of the present disclosure. Ultrafiltration utilizes ultrafiltration membranes, with pore sizes typically between 0.001 and 0.1 μm. Ultrafiltration membranes can also be defined by their molecular weight cutoff (MWCO) and can have a range from 1 kD to 500 kD. Ultrafiltration is generally used for concentrating and formulating dissolved biomolecules such as proteins, peptides, plasmids, viral particles, nucleic acids, and carbohydrates. Ultrafiltration systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, one or more nanofiltration steps may be used during clarification and purification. Nanofiltration utilizes nanofiltration membranes, with pore sizes typically less than 100 nm. Nanofiltration is generally used for removal of unwanted endogenous viral impurities (e.g. baculovirus). In certain embodiments, nanofiltration can comprise viral removal filtration (VRF). VRF filters can have a filtration size typically between 15 nm and 100 nm. Examples of VIZ filters comprise (but are not limited to): Planova 15N, Planova 20N, and Planova 35N (Asahi-Kasei Corp, Tokyo, Japan); and Viresolve NFP and Viresolve NFR (Millipore Corp, Billerica, Mass., USA). Nanofiltration systems of the present disclosure can be pre-rinsed, packed, equilibrated, flushed, processed, eluted, washed or cleaned with formulations known to those in the art, comprising AAV pharmaceutical, processing and storage formulations of the present disclosure. In certain embodiments, nanofiltration is used to remove aggregated clumps of viral particles.

In certain embodiments, one or more tangential flow filtration (TFF) (also known as cross-flow filtration) steps may be used during clarification and purification, Tangential flow filtration is a form of membrane filtration in which a feed stream (which comprises the target agent/particle to be clarified and concentrated) flows from a feed tank into a filtration module or cartridge, Within the TFF filtration module, the feed stream passes parallel to a membrane surface, such that one portion of the stream passes through the membrane (permeate/filtrate) while the remainder of the stream (retentate) is recirculated back through the filtration system and into the feed tank.

In certain embodiments, the TFF filtration module can be a flat plate module (stacked planar cassette), a spiral wound module (spiral-wound membrane layers), or a hollow fiber module (bundle of membrane tubes). Examples of TFF systems for use in the present disclosure comprise, but are not limited to: Spectrum mPES Hollow Fiber TFF system (0.5 mm fiber ID, 100 kDA MWCO) or Millipore Ultracel PLCTK system with Pellicon-3 cassette (0.57 m², 30 kDA INIWCO).

New buffer materials can be added to the TFF feed tank as the feed stream is circulated through the TFF filtration system. In certain embodiments, buffer materials can be fully replenished as the flow stream circulates through the TFF filtration system. In this embodiment, buffer material is added to the stream in equal amounts to the buffer material lost in the permeate, resulting in a constant concentration. In certain embodiments, buffer materials can be reduced as the flow stream circulates through the filtration system. In this embodiment, a reduced amount of buffer material is added to the stream relative to the buffer material lost in the permeate, resulting in an increased concentration. In certain embodiments, buffer materials can be replaced as the flow stream circulates through the filtration system. In this embodiment, the buffer added to stream is different from buffer materials lost in the permeate, resulting in an eventual replacement of buffer material in the stream. TFF systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, a TFF load pool can be spiked with an excipient or diluent prior to filtration. In certain embodiments, a TFF load pool is spiked with a high-salt mixture (such as sodium chloride or potassium chloride) prior to filtration. in certain embodiments, a TFF load pool is spiked with a high-sugar mixture (such as 50% w/v sucrose) prior to filtration.

The effectiveness of TFF processing can depend on several factors, comprising (but not limited to): shear stress from flow design, cross-flow rate, filtrate flow control, transmembrane pressure (TMP), membrane conditioning, membrane composition (e.g. hollow fiber construction) and design (e.g. surface area), system flow design, reservoir design, and mixing strategy. In certain embodiment, the filtration membrane can be exposed to pre-TFF membrane conditioning.

In certain embodiments, TFF processing can comprise one or more microfiltration stages. In certain embodiments, TFF processing can comprise one or more ultrafiltration stages. In certain embodiments, TFF processing can comprise one or more nanofiltration stages.

In certain embodiments, TFF processing can comprise one or more concentration stages, such as an ultrafiltration (UF) or microfiltration (MF) concentration stage. In the concentration stage, a reduced amount of buffer material is replaced as the stream circulates through the filtration system (relative to the amount of buffer material lost as permeate). The failure to completely replace all of the buffer material lost in the permeate results in an increased concentration of viral particles within the filtration stream. In certain embodiments, an increased amount of buffer material is replaced as the stream circulates through the filtration system. The incorporation of excess buffer material relative to the amount of buffer material lost in the permeate results in a decreased concentration of viral particles within the filtration stream.

In certain embodiments, TFF processing can comprise one or more diafiltration (DF) stages. The diafiltration stage comprises replacement of a first buffer material (such as a high salt material) within a second buffer material (such a low-salt or zero-salt material). In this embodiment, a second buffer is added to flow stream which is different from a first buffer material lost in the permeate, resulting in an eventual replacement of buffer material in the stream.

In certain embodiments, TFF processing can comprise multiple stages in series. In certain embodiments, a TFF processing process can comprise an ultrafiltration (UF) concentration stage followed by a diafiltration stage (DF). In certain embodiments, a TFF processing can comprise a diafiltration stage followed by an ultrafiltration concentration stage. In certain embodiments, a TFF processing can comprise a first diafiltration stage, followed by an ultrafiltration concentration stage, followed by a second diafiltration stage. In certain embodiments, a TFF processing can comprise a first diafiltration stage which incorporates a high-salt-low-sugar buffer material into the flow stream, followed by an ultrafiltration/concentration stage which results in a high concentration of the viral material in the flow stream, followed by a second diafiltration stage which incorporates a low-salt-high-sugar or zero-salt-high-sugar buffer material into the flow stream. In certain embodiments, the salt can he sodium chloride, sodium phosphate, potassium chloride, potassium phosphate, or a combination thereof. In certain embodiments, the sugar can be sucrose, such as a 5% w/v sucrose mixture or a 7% w/v sucrose mixture.

In certain embodiments, the one or more TFF steps can comprise a formulation diafiltration step in which at least a portion of the liquid media of the viral production pool is replaced with a high-sucrose formulation buffer. In certain embodiments, the high-sucrose formulation buffer comprises between 6-8% w/v of a sugar or sugar substitute and between 90-100 mM of an alkali chloride salt. In certain embodiments, the high-sucrose formulation buffer comprises 7% w/v of sucrose and between 90-100 mM sodium chloride. In certain embodiments, the high-sucrose formulation buffer comprises 7% w/v of sucrose. 10 mM Sodium Phosphate, between 95-100 sodium chloride, and 0.001% (w/v) Poloxamer 188. In certain embodiments, the formulation diafiltration step is the final diafiltration step in the one or more TFF steps. In certain embodiments, the formulation diafiltration step is the only diafiltration step in the one or more TFF steps.

In certain embodiments, TFF processing can comprise multiple stages which occur contemporaneously. As a non-limiting example, a TFF clarification process can comprise an ultrafiltration stage which occurs contemporaneously with a concentration stage.

Methods of cell lysate clarification and purification by filtration are well understood in the art and may be carried out according to a variety of available methods comprising, but not limited to passive filtration and flow filtration. Filters used may comprise a variety of materials and pore sizes. For example, cell lysate filters may comprise pore sizes of from about 1 μM to about 5 μM, from about 0.5 μM to about 2 μM, from about 0.1 μM to about 1 μM, from about 0.05 μM to about 0.05 μM and from about 0.001 μM to about 0.1 μM. Exemplary pore sizes for cell lysate filters may comprise, but are not limited to, 2.0, 1.9, 1.8, 1.7, 1.6, 1.5, 1.4, 1.3, 1.2, 1.1, 1, 0.9. 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In certain embodiments, clarification may comprise filtration through a filter with 2.0 μM pore size to remove large debris, followed by passage through a filter with 0.45 μM pore size to remove intact cells.

Filter materials may be composed of a variety of materials. Such materials may comprise, but are not limited to, polymeric materials and metal materials (e.g. sintered metal and pored aluminum.) Exemplary materials may comprise, but are not limited to nylon, cellulose materials (e.g. cellulose acetate), polyvinylidene fluoride (PVDF), polyethersulfone, polyamide, polysulfone, polypropylene, and polyethylene terephthalate. In certain embodiments, filters useful for clarification of cell lysates may comprise, but are not limited to ULTIPLEAT PROFILE™ filters (Pall Corporation, Port Washington, N.Y.), SUPORIM membrane filters (Pall Corporation, Port Washington, N.Y.).

In certain embodiments, flow filtration may be carried out to increase filtration speed and/or effectiveness. In certain embodiments, flow filtration may comprise vacuum filtration. According to such methods, a vacuum is created on the side of the filter opposite that of cell lysate to be filtered. In certain embodiments, cell lysates may be passed through filters by centrifugal forces. In certain embodiments, a pump is used to force cell lysate through clarification filters. Flow rate of cell lysate through one or more filters may he modulated by adjusting one of channel size and/or fluid pressure.

### Clarification and Purification: Chromatography

In certain embodiments, AAV particles in a formulation may be clarified and purified from cell lysates through one or more chromatography steps using one or more different methods of chromatography. Chromatography refers to any number of methods known in the art for selectively separating out one or more elements from a mixture. Such methods may comprise, but are not limited to, ion exchange chromatography (e.g, cation exchange chromatography and anion exchange chromatography), affinity chromatography (e.g. immunoaffinity chromatography, metal affinity chromatography, pseudo affinity chromatography such as Blue Sepharose resins), hydrophobic interaction chromatography (HIC), size-exclusion chromatography, and multimodal chromatography (MMC) (chromatographic methods that utilize more than one form of interaction between the stationary phase and analytes). in certain embodiments, methods or systems of viral chromatography may comprise any of those taught in U.S. Pat. Nos, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Chromatography systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, one or more ion exchange (IEX) chromatography steps may be used to isolate viral particles. The ion exchange step can comprise anion exchange (AEX) chromatography, cation exchange (CEX) chromatography, or a combination thereof In certain embodiments, ion exchange chromatography is used in a bind/elute mode. Bind/elute IEX can he used by binding viral particles to a stationary phase based on charge-charge interactions between capsid proteins (or other charged components) of the viral particles and charged sites present on the stationary phase. This process can comprise the use of a column through which viral preparations (e.g. clarified lysates) are passed. After application of viral preparations to the charged stationary phase (e.g, column), bound viral particles may then be elated from the stationary phase by applying an elution solution to disrupt the charge-charge interactions. Elution solutions may be optimized by adjusting salt concentration and/or pH to enhance recovery of hound viral particles. In certain embodiments, the elution solution can comprise a nuclease such as Fienzonase nuclease. Depending on the charge of viral capsids being isolated, cation or anion exchange chromatography methods may be selected. In certain embodiments, ion exchange chromatography is used in a flow-through mode, Flow-through IEX can he used by binding non-viral impurities or unwanted viral particles to a stationary phase (based on charge-charge interactions) and allowing the target viral particles in the viral preparation to “flow through” the IEX system into a collection pool.

Methods or systems of ion exchange chromatography may comprise, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety.

In certain embodiments, the TEX process uses an AEX chromatography system such as a Sartorius Sartobind Q membrane, a Sartorius Sartohind STIC membrane, a Millipore Fractogel TMAE HiCap(m) Flow-Through membrane, a GE Q Sepharose HP membrane, Poros XQ or Poros HQ. In certain embodiments, the IEX process uses a CEX system such as a Poros XS membrane. In certain embodiments, the AEX system comprises a stationary phase which comprises a trimethylammoniumethyl (TMAE) functional group. In certain embodiments, the IEX process uses a Multimodal Chromatography (MMC) system such as a Nuvia aPrime 4A membrane.

In certain embodiments, one or more affinity chromatography steps, such as immunoaffinity chromatography, may be used to isolate viral particles. Immunoaffinity chromatography is a form of chromatography that utilizes one or more immune compounds (e.g. antibodies or antibody-related structures) to retain viral particles. Immune compounds may bind specifically to one or more structures on viral particle surfaces, comprising, but not limited to one or more viral coat protein. In certain embodiments, immune compounds may be specific for a particular viral variant. In certain embodiments, immune compounds may bind to multiple viral variants. In certain embodiments, immune compounds may comprise recombinant single-chain antibodies. Such recombinant single chain antibodies may comprise those described in Smith, R. H. et al., 2009. Mol. Ther. 17(11):1888-96, the contents of which are herein incorporated by reference in their entirety. Such immune compounds are capable of binding to several AAV capsid variants, comprising, but not limited to AAV1, AAV2, AAV6 and AAV8 or any of those taught herein. In certain embodiments, the AFC process uses a GE AVB Sepharose HP column resin, Poros CaptureSelect AAV8 resins (ThermoFisher), Poros CaptureSelect AAV9 resins (ThermoFisher) and Poros CaptureSelect AAVX resins (ThermoFisher).

In certain embodiments, one or more size-exclusion chromatography (SEC) steps may be used to isolate viral particles. SEC may comprise the use of a gel to separate particles according to size. In viral particle purification, SEC filtration is sometimes referred to as “polishing.” In certain embodiments, SEC may be carried out to generate a final product that is near-homogenous. Such final products may in certain embodiments be used in pre-clinical studies and/or clinical studies (Kotin, R. M. 2011. Human Molecular Genetics. 20(1):R2-R6, the contents of which are herein incorporated by reference in their entirety.) In certain embodiments, SEC may be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

### III. COMPOSITIOSN AND FORMULATIONS

### General

Gene therapy drug products (such as rAAV particles) are challenging to incorporate into composition and formulations due to their limited stability in the liquid state and a high propensity for large-scale aggregation at low concentrations. Gene therapy drug products are often delivered directly to treatment areas (comprising CNS tissue); which requires that excipients and formulation parameters be compatible with tissue function, microenvironment, and volume restrictions.

According to the present disclosure. AAV particles may he prepared as, or comprised in, pharmaceutical compositions. It will be understood that such compositions necessarily comprise one or more active ingredients and, most often, one or more pharmaceutically acceptable excipients.

Relative amounts of the active ingredient (e.g. AAV particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% v/w) of the active ingredient. By way of example, the composition may comprise between 0.1% and 100%, e.g., between .5 and 50%, between 1-30%, between 5-80%, or at least 80% (w/w) active inuedient.

In certain embodiments, the AAV particle pharmaceutical compositions described herein may comprise at least one payload of the present disclosure. As a non-limiting example, the pharmaceutical compositions may contain an AAV particle with 1, 2, 3, 4 or 5 payloads.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated comprise, but are not limited to, humans and; or other primates; mammals, comprising commercially relevant mammals such as cattle, pigs, horses, sheep, cats, dogs, mice, rats, birds, comprising commercially relevant birds such as poultry, chickens, ducks, geese, and/or turkeys.

In certain embodiments, compositions are administered to humans, human patient or subjects.

Formulations of the present disclosure can comprise, without limitation, saline, liposomes, lipid nanoparticles, polymers, peptides, proteins, cells transfected with AAV particles (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods comprise the step of associating the active ingredient with an excipient and/or one or more other accessory ingredients. As used herein, the phrase “active ingredient” generally refers either to an AAV particle carrying a payload rethon encoding the polynucleotide or polypeptides of the present disclosure or to the end product encoded by a viral genome of an AAV particle as described herein.

In certain embodiments, the formulations may comprise at least one inactive ingredient. As used herein, the term “inactive ingredient” refers to one or more inactive agents comprised in formulations. In certain embodiments, all, none or some of the inactive ingredients which may be used in the formulations of the present disclosure may be approved by the US Food and Drug Administration (FDA).

Formulations of the AAV particles and pharmaceutical compositions described herein may be prepared by any method known or hereafter developed in the art of pharmacology. In general, such preparatory methods comprise the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In certain embodiments, formulations of the present disclosure are aqueous formulations (i.e. formulations which comprise water). In certain embodiments, formulations of the present disclosure comprise water, sanitized water, or Water-for-injection (WFI).

In certain embodiments, the AAV particles of the present disclosure may be formulated in PBS with 0.001%-0.1% (w/v) of Poloxamer 188 (e.g. Pluronic F-68) at a pH of about 7.0.

In certain embodiments, the , AV formulations described herein may contain sufficient AAV particles for expression of at least one expressed functional payload. As a non-limiting example, the AAV particles may contain viral genomes encoding 1, 2, 3, 4 or 5 functional payloads.

According to the present disclosure AAV particles may be formulated for CNS delivery. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

In certain embodiments, the AAV formulations described herein may comprise a buffering system which comprises phosphate, Tris, and/or Histidine. The buffering agents of phosphate, Tris, and/or Histidine may be independently used in the formulation in a range of 2-12 mM.

Formulations of the present disclosure can be used in any step of producing, processing, preparing, storing, expanding, or administering AAV particles and viral vectors of the present disclosure. in certain embodiments, pharmaceutical formulations and components can be use in AAV production, AAV processing, AAV clarification, AAV purification, and AAV finishing systems of the present disclosure, all of which can be pre-rinsed, packed, equilibrated, flushed, processed, eluted, washed or cleaned with formulations known to those in the art, comprising AAV pharmaceutical, processing and storage formulations of the present disclosure.

### Excipients and Diluents

The AAV particles of the present disclosure can be formulated into a pharmaceutical composition which comprises one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload of the present disclosure.

Relative amounts of the active ingredient (e.g. AAV particle), the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. in certain embodiments, the composition may comprise between 0.001% and 99% (w/w) of the active ingredient. By way of example, the composition may comprise between 0.001% and 100%, e.g., between 0.5 and 50%, between 1-30%, between 5-80%, or at least 80% (w/w) active ingredient. In certain embodiments, the composition may comprise between 0.001% arid 99% (w/w) of the excipients and diluents. By way of example, the composition may comprise between 0.001% and 100%, e.g., between 0.5 and 50%, between 1-30%, between 5-80%, or at least 80% (w/w) excipients and diluents.

In certain embodiments, a pharmaceutically acceptable excipient may be at least 95%, at least 96%, at least 97%, at least 98%, at least 99%, or 100% pure, In certain embodiments, an excipient is approved for use for humans and for veterinary use. In certain embodiments, an excipient may be approved by United States Food and Drug Administration. In certain embodiments, an excipient may be of pharmaceutical grade. In certain embodiments, an excipient may meet the standards of the United States Pharmacopoeia (USP), the European Pharmacopoeia (EP), the British Pharmacopoeia, and/or the International Pharmacopoeia.

Excipients, as used herein, comprise, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety), The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary excipients and diluents which can be comprised in formulations of the present disclosure comprise, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary excipients and diluents which can be comprised in formulations of the present disclosure comprise, but are not limited to, 1,2,6-fiexanetriol; 1,2-Dirrivristovi-Sn-Glycero-3-(Phospho-S-(1-Glycerol)); 1,2-Dimyristoyl-Sn-Glycero-3-Phosphocholine; 1,2-Diolcoyl-Sn-Glycero-3-Phosphocholine; 1,2-Dipalmitoyl-Sn-Glycem-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-O-Tolyibiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A103; Adhesive Tape; Adipic Acid; Aerotex Resin 3730; Alanine; Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl Alpha-Ionone, Almond Oil; Alpha-Terpineol; Alpha-Tocopherol; Alpha-Tocopherol Acetate, Dl-; Alpha-Tocopherol, Dl-; Aluminum Acetate; Aluminum Chlorhydroxy Allantoinate; Aluminum Hydroxide; Aluminum Hydroxide—Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Pea-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C20-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric. Triglyceride; Captylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa Butter; Coco-Glycerides; Coconut Oil; Coconut Oil, Hydrogenated; Coconut Oil/Palm Kernel Oil Glycerides, Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, D1-; D&C Red No. 28; D&C Red No. 33; D&C Red No. 36; D&C Red No. 39; D&C Yellow No, 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylarninoethyl Methacrylate-Butyl Methacrylate-Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatidylalycerol, D1-; Dipropylene. Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetate Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Oleate; Ethylcelluloses; Ethylene Glycol; Ethylene Vinyl Acetate Copolymer; Ethylenediamine; Ethylenediamine Dihydrochlaride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazine; Fat, Edible; Fat, Hard; Fatty Acid Esters; Fatty Acid Pentaerythriol Ester; Fatty Acids; Fatty Alcohol Citrate; Fatty Alcohols; H&C Blue No. 1; Fd&C Green No. 3; Fd&C Red No. 4; Fd&C Red No. 40; Fd&C Yellow No. 10 (Delisted); Fd&.0 Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor FIG. 827118; Flavor Raspberry Pfc-8407; Flavor Rhodia Pharmaceutical No. Rf 451; Fluorochlomhydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498g; Fragrance Balsam Pine No. 5124; Fragrance Bouquet 10328; Fragrance Chemodemi 6401-B; Fragrance Chemodemi 6411; Fragrance Cream No. 73457; Fragrance Cs-28197; Fragrance Felton 066m; Fragrance Firmenich 47373; Fragrance Givaudan Ess 9090/1c; Fragrance H-6540; Fragrance Herbal 10396; Fragrance Nj-1085; Fragrance P O F1-147; Fragrance Pa 52805; Fragrance Pera Denn D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoarn Sponge; Cellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, D1-; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate-Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm./Palm Kernel Oil. Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosanyl Hydroxystearate; Hydroxypropyl Cellulose; Hydroxypropyl Methylcellulose 2906; Hydroxypropyl-Beta-cyclodextrin; Hypromellose 2208 (15000 Mpa.S); Hypromellose 2910 (15000 Mpa.S); Hypromelloses; Imidurea; Iodine; Todoxamic Acid; Iofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate—Myristyl Alcohol; Isopropyl Palmitate; Isopropyl Stearate; isostearic Acid; isostearyl Alcohol; Isotonic Sodium Chloride Solution; Jelene; Kaolin; Kathon Cg; Kathon Cg II; Lactate; Lactic Acid; Lactic Acid, DI-; Lactic Acid, L-; Lactohionic Acid; Lactose; Lactose Monohydrate; Lactose, Hydrous; Laneth; Lanolin; Lanolin Alcohol—Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdirnonium Hydrolyzed Animal Collagen; Laureth Sulfate; Laureth-2; Laureth-23; Laureth-4; Laurie Diethanolamide; Laurie Myristic Diethanolamide; Lattroyl Sarcosine; Lauryl Lactate; Lauryl Sulfate; Lavandula Angustifolia Flowering Top; Lecithin; Lecithin Unblea.ched; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+1-)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified S-15; Medical Antifonn A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa.S); Mahylcelluloses; Methylchloroisothiazolinone; Methylene Blue; Methylisothiazolinone; Methylparaben; Microcrystalline Wax, Mineral Oil; Mono And Dialyceride; Monostearyl Citrate; Monothioglycerol; Multisterol Extract; Myristyl Alcohol; Myristyl Lactate; Myristyl-Gamma-Picolinium Chloride; N-(Carbamoyl-Methoxy Peg-40)-1,2-Distearoyl-Cephalin Sodium; N,N-Dimethylacetatnide; Niacinamide; Nioxime; Nitric Acid; Nitrogen; Nonoxynol Iodine; _Nonoxynol-15; Nonoxynol-9; Norflurane; Oatmeal; Octadecene-1/Maleic Acid Copolymer; Octanoic Acid; Octisalate; Octoxynol-1; Octoxynol-40; Octoxynol-9; Octyldodecanol; Octylphenol Polymethylene; Oleic Acid; Oleth-10/Oleth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Paifum Creme 45/3; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg-120 Glyceryl Stearate; Peg-120 Methyl Glucose Dioleate; Peg-15 Cocamine; Peg-150 Distearate; Peg-2 Stearate; Peg-20 Sorbitan Isostearate; Pea-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90g; Phosphoric Acid; Pine Needle Oil (Pinus Sylvestris); Piperazine Hexahydrate; Pla.stibase-50w; Polacrilin; Polidronium Chloride; Poloxamer 124; Poloxamer 181; Poloxamer 182; Poloxamer 188; Poloxamer 237; .Poloxamer 407; Poly(Bis(P-Carboxyphenoxy)Propane Anhydride): Sebacic Acid; Poly(Dimethylsiloxane/Methylvinylsiloxane/Methylhydrogensiloxane) Dimethylvinyl Or Dimethylhydroxy Or Trimethyl Endblocked; Poly(D1-Lactic-Co-Glycolic Acid), (50:50; Poly(DI-Lactic-Co-Glycolic Acid), Ethyl Ester Terminated, (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyalactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene-Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxi,71Distearate; Polyoxyl Glyceryl Stearate; Polyoxyl Lanolin; Polyoxyl Palmitate; Polyoxyl Stearate; Polypropylene; Polypropylene Glycol; Polyquatemium-10; Polyquatemium-7 (70/30 Acrylamide/Dadmac; Polysiloxane; Polysorbate 20; Polysorbate 40; Polysorbate 60; Polysorbate 65, Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate, Potassium Bisuifite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorhate; Povidone Acrylate Copolymer; Povidone Hydrogel; Povidone K17; Povidone K25; Povidone K29/32; Povidone K30; Povidone K90; Povidone K90f, Povidone/Eicosene Copolymer, Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; PramIgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl; Urea/Methylpaniben/Propylparben; Propylparaben; Protamine Sulfate; Protein Hydrolysate; Pvin/Ma Copolymer; Quatemium-15; Quaternium-15 Cis-Form; Quatemium-52; Ra-2397; Ra-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine, Sesame Oil; Shea Butter; Silastic Brand Medical Grade Tubing; Silastic Medical Adhesive,Silicone Type A; Silica, Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated UndecycleniclYlonoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; SorbitanlYlonolaurate; SorbitanlYlonooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalene; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10; Steareth-100; Steareth-2; Steareth-20; Steareth-2I; Steareth-40; Stearic. Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether .Beta.-Cyclodextrin; Sulfur Dioxide; Sulfuric Acid; Sulfurous Acid; Surfactol Qs; Tagatose, D-; Talc; Tall Oil; Tallow Glycerides; Tartaric Acid; Tartaric Acid, D1-; Tenox; Tenox-2; Tert-Butyl Alcohol; Tert-Butyl Hydroperoxide; Tert-Butylhydroquinone; Tetrakis(2-Methoxvisobutylisocyanide)Copper(i) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodiwn Citrate Dihydrate; Trisodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

Pharmaceutical formulations of AAV particles disclosed herein may comprise cations or anions. In certain embodiments, the formulations comprise metal cations such as, but not limited to, Zn²⁺, Ca²⁺, Cu²⁺, Mn²⁺, Mg⁺ and combinations thereof. As a non-limiting example, formulations may comprise polymers and complexes with a metal cation (See e.g., U.S. Pat. Nos. 6,265,389 and 6,555,525, each of which is herein incorporated by reference in its entirety).

Formulations of the present disclosure may also comprise one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid).

In certain embodiments, additional excipients that may be used in formulating the pharmaceutical composition may comprise magnesium chloride (MgCl₂), arginine, sorbitol, and/or trehalose.

Formulations of the present disclosure may comprise at least one excipient and/or diluent in addition to the AAV particle. The formulation may comprise 1, 2, 3, 4, 5, 6, 7, 8, 9, 10, or more than 10 excipients and/or diluents in addition to the AAV particle.

In certain embodiments, the formulation may comprise, but is not limited to, phosphate-buffered saline (PBS). As a non-limiting example, the PBS may comprise sodium chloride, potassium chloride, disodium phosphate, monopotassium phosphate, and distilled water. In some instances, the PBS does not contain potassium or magnesium. In other instances, the PBS contains calcium and magnesium.

### Sodium Phosphate

In certain embodiments, at least one of the components in the formulation is sodium phosphate. The formulation may comprise monobasic, dibasic or a combination of both monobasic and dibasic sodium phosphate.

In certain embodiments, the concentration of sodium phosphate in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10,6 mM. 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12,6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may comprise sodium phosphate in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 moi, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM. 8.6-9.1 mM, 8.7-9.2 mM, 8,8-9.3 mM, 89-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM. 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In certain embodiments, the formulation may comprise 0-10 mM of sodium phosphate.

In certain embodiments, the formulation may comprise 2-12 mM of sodium phosphate.

In certain embodiments, the formulation may comprise 2-3 mM of sodium phosphate.

In certain embodiments, the formulation may comprise 9-10 mM of sodium phosphate.

In certain embodiments, the formulation may comprise 10-11 mM of sodium phosphate.

In certain embodiments, the formulation may comprise 2.7 mM of sodium phosphate.

In certain embodiments, the formulation may comprise 10 mM of sodium phosphate,

### Potassium Phosphate

In certain embodiments, at least one of the components in the formulation is potassium phosphate. The formulation may comprise monobasic, dibasic or a combination of both monobasic and dibasic potassium phosphate.

In certain embodiments, the concentration of potassium phosphate in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may comprise potassium phosphate in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, mM, mM, 5.3-5.8 mM, 5.4-5.9 mM, 5.5-6 mM. 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12,6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM- 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In certain embodiments, the formulation may comprise 0-10 mM of potassium phosphate.

In certain embodiments, the formulation may comprise 1-3 mM of potassium phosphate.

In certain embodiments, the formulation may comprise 1-2 mM of potassium phosphate.

In certain embodiments, the formulation may comprise 2-3 mM of potassium phosphate.

In certain embodiments, the formulation may comprise 2-12 mM of potassium phosphate.

In certain embodiments, the formulation may comprise 1.5 mM of potassium phosphate. As a non-limiting example, the formulation may comprise 1.54 mM of potassium phosphate.

In certain embodiments, the formulation may comprise 2 mM of potassium phosphate.

### Sodium Chloride

In certain embodiments, at least one of the components in the formulation is sodium chloride.

In certain embodiments, the concentration of sodium chloride in a formulation may be, but is not limited to, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 101 mM, 102 mM, 103 mM, 104 mM, 105 mM, 106 mM, 107 mM, 108 mM, 109 mM, 110 mM, 111 mM, 112 mM, 113 mM, 114 mM, 115 mM, 116 mM, 117 mM, 118 mM, 119 mM, 120 mM, 121 mM, 122 mM, 123 mM, 124 mM, 125 mM, 126 mM, 127 mM, 128 mM, 129 mM, 130 mM, 131 mM, 132 mM, 133 mM, 134 mM, 135 mM, 136 mM, 137 mM, 138 mM, 139 mM, 140 mM, 141 mM, 142 mM, 143 mM, 144 mM, 145 mM, 146 mM, 147 mM, 148 mM, 149 mM, 150 mM, 151 mM, 152 mM, 153 mM, 154 mM, 155 mM, 156 mM, 157 mM, 158 mM, 159 mM, 160 mM, 161 mM, 162 mM, 163 mM, 164 mM, 165 mM, 166 mM, 167 mM, 168 mM, 169 mM, 170 mM, 171 mM, 172 mM, 173 mM, 174 mM, 175 mM, 176 mM, 177 mM, 178 mM, 179 mM, 180 mM, 181 mM, 182 mM, 183 mM, 184 mM, 185 mM, 186 mM, 187 mM, 188 mM, 189 mM, 190 mM, 191 mM, 192 mM, 193 mM, 194 mM, 195 mM, 196 mM, 197 mM, 198 mM, 199 mM, 200 mM, 201 mM, 202 mM, 203 mM, 204 mM, 205 mM, 206 mM, 207 mM, 208 mM, 209 mM, 210 mM, 211 mM, 212 mM, 213 mM, 214 mM, 215 mM, 216 mM, 217 mM, 218 mM, 219 mM, or 220 mM.

The formulation may comprise sodium chloride in a range of 7 -85 mM, 80-90 mM, 85-95 mM, 90-100 mM, 95-105 mM, 100-110 mM, 105-115 m 110-120 mM, 115-125 mM, 120-130 mM, 125-135 mM, 130-140 mM, 135-145 mM, 140-150 mM, 145-155 mM, 150-160 mM, 155-165 mM, 160-170 mM, 165-175 mM, 170-180 mM, 175-185 mM, 180-190 mM, 185-195 mM, 190-200 mM, 75-95 mM, 80-100 mM, 85-105 mM, 90-110 mM, 95-115 mM, 100-120 mM, 105-125 mM, 110-130 mM, 115-135 mM, 120-140 mM, 125-145 mM, 130-150 mM, 135-155 mM, 140-160 mM, 145-165 mM, 150-170 mM, 155-175 mM, 160-180 mM, 165-185 mM, 170-190 mM, 175-195 mM, 180-200 mM, 75-100 mM, 80-105 mM, 85-110 mM, 90-115 mM, 95-120 mM, 100-125 mM, 105-130 mM, 110-135 mM, 115-140 mM, 120-145 mM, 125-150 mM, 130-155 mM, 135-160 mM, 140-165 mM, 145-170 mM, 150-175 mM, 155-180 mM, 160-185 mM, 165-190 mM, 170-195 mM, 175-200 mM, 75-105 mM, 80-110 mM, 85-115 mM, 90-120 mM, 95-125 mM, 100-130 mM, 105-135 mM, 110-140 mM, 115-145 mM, 120-150 mM, 125-155 mM, 130-160 mM, 135-165 mM, 140-170 mM, 145-175 mM, 150-180 mM, 155-185 mM, 160-190 mM, 165-195 mM, 170-200 mM, 75-115 mM, 80-120 mM, 85-125 mM, 90-130 mM, 95-135 mM, 100-140 mM, 105-145 mM, 110-150 mM, 115-155 mM, 120-160 mM, 125-165 mM, 130-170 mM, 135-175 mM, 140-180 mM, 145-185 mM, 150-190 mM, 155-195 mM, 160-200 mM, 75-120 mM, 80-125 mM, 85-130 mM, 90-135 mM, 95-140 mM, 100-145 mM, 105-150 mM, 110-155 mM, 115-160 mM, 120-165 mM, 125-170 mM, 130-175 mM, 135-180 mM, 140-185 mM, 145-190 mM, 150-195 mM, 155-200 mM, 75-125 mM, 80-130 mM, 85-135 mM, 90-140 mM, 95-145 mM, 100-150 mM, 105-155 mM, 110-160 mM, 115-165 mM, 120-170 mM, 125-175 mM, 130-180 mM, 135-185 mM, 140-190 mM, 145-195 mM, 150-200 mM, 75-125 mM, 80-130 mM, 85-135 mM, 90-140 mM, 95-145 mM, 100-150 mM, 105-155 mM, 110-160 mM, 115-165 mM, 120-170 mM, 125-175 mM, 130-180 mM, 135-185 mM, 140-190 mM, 145-195 mM, 150-200 mM, 75-135 mM, 80-140 mM, 85-145 mM, 90-150 mM, 95-155 mM, 100-160 mM, 105-165 mM, 110-170 mM, 115-175 mM, 120-180 mM, 125-185 mM, 130-190 mM, 135-195 mM, 140-200 mM, 75-145 mM, 80-150 mM, 85-155 mM, 90-160 mM, 95-165 mM, 100-170 mM, 105-175 mM, 110-180 mM, 115-185 mM, 120-190 mM, 125-195 mM, 130-200 mM, 75-155 mM, 80-160 mM, 85-165 mM, 90-170 mM, 95-175 mM, 100-180 mM, 105-185 mM, 110-190 mM, 115-195 mM, 120-200 mM, 75-165 mM, 80-170 mM, 85-175 mM, 90-180 mM, 95-185 mM, 100-190 mM, 105-195 mM, 110-200 mM, 75-175 mM, 80-180 mM, 85-185 mM, 90-190 mM, 95-195 mM, 100-200 mM, 80-220 mM, 90-220 mM, 100-220 mM, 110-220 mM, 120-220 mM, 130-220 mM, 140-220 mM, 150-220 mM, 160-220 mM, 170-220 mM, 180-220 mM, 190-220 mM, 200-220 mM, or 210-220 mM.

In certain embodiments, the formulation may comprise 80-220 mM of sodium chloride.

In certain embodiments, the formulation may comprise 80-150 mM of sodium chloride.

In certain embodiments, the formulation may comprise 75 mM of sodium chloride.

In certain embodiments, the formulation may comprise 83 mM of sodium chloride.

In certain embodiments, the formulation may comprise 92 mM of sodium chloride.

In certain embodiments, the formulation may comprise 95 mM of sodium chloride.

In certain embodiments, the formulation may comprise 98 mM of sodium chloride

In certain embodiments, the formulation may comprise 100 mM of sodium chloride.

In certain embodiments, the formulation may comprise 107 mM of sodium chloride.

In certain embodiments, the formulation may comprise 109 mM of sodium chloride.

In certain embodiments, the formulation may comprise 118 mM of sodium chloride.

In certain embodiments, the formulation may comprise 125 mM of sodium chloride.

In certain embodiments, the formulation may comprise 127 mM of sodium chloride.

In certain embodiments, the formulation may comprise 133 mM of sodium chloride.

In certain embodiments, the formulation may comprise 142 mM of sodium chloride.

In certain embodiments, the formulation may comprise 150 mM of sodium chloride

In certain embodiments, the formulation may comprise 155 mM of sodium chloride.

In certain embodiments, the formulation may comprise 180 mM of sodium chloride.

In certain embodiments, the formulation may comprise 192 mM of sodium chloride.

In certain embodiments, the formulation may comprise 210 mM of sodium chloride.

### Potassium Chloride

In certain embodiments, at least one of the components in the formulation is potassium chloride.

In certain embodiments, the concentration of potassium chloride in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7,5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 m.M, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 1.4 mM, 14.1 mM, 14.2 mM, 14.3 mM, 14.4 mM, 14.5 mM, 14.6 mM, 14.7 mM, 14.8 mM, 14.9 mM or 15 mM.

The formulation may comprise potassium chloride in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM. 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14,7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM- 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In certain embodiments, the formulation may comprise 0-10 mM of potassium chloride.

In certain embodiments, the formulation may comprise 1-3 mM of potassium chloride.

In certain embodiments, the formulation may comprise 1-2 mM of potassium chloride.

In certain embodiments, the formulation may comprise 2-3 mM of potassium chloride.

In certain embodiments, the formulation may comprise 1.5 mM of potassium chloride.

In certain embodiments, the formulation may comprise 2.7 mM of potassium chloride.

### Magnesium Chloride

In certain embodiments, at least one of the components in the formulation is magnesium chloride.

In certain embodiments, the concentration of magnesium chloride may be, but is not limited to, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, or 100 mM.

The formulation may comprise magnesium chloride in a range of 0-5 mM, 1-5 mM, 2-5 mM, 3-5 mM, 4-5 mM, 0-10 mM, 1-10 mM, 2-10 mM, 3-10 mM, 4-10 mM, 5-10 mM, 6-10 mM, 7-10 mM, 8-10 mM, 9-10 mM, 0-25 mM, 1-25 mM, 2-25 mM, 3-25 mM, 4-25 mM, 5-25 mM, 6-25 mM, 7-25 mM, 8-25 mM, 9-25 mM, 10-25 mM, 11-25 mM, 12-25 mM, 13-25 mM, 14-25 mM, 15-25 mM, 16-25 mM, 17-25 mM, 18-25 mM, 19-25 mM, 20-25 mM, 21-25 mM, 22-25 mM, 23-25 mM, 24-25 mM, 0-50 mM, 1-50 mM, 2-50 mM, 3-50 mM, 4-50 mM, 5-50 mM, 6-50 mM, 7-50 mM, 8-50 mM, 9-50 mM, 10-50 mM, 11-50 mM, 12-50 mM, 13-50 mM, 14-50 mM, 15-50 mM, 16-50 mM, 17-50 mM, 18-50 mM, 19-50 mM, 20-50 mM, 21-50 mM, 22-50 mM, 23-50 mM, 24-50 mM, 25-50 mM, 26-50 mM, 27-28-50 mM, 29-50 mM, 30-50 mM, 31-50 mM, 32-50 mM, 33-50 mM, 34-50 mM, 35-50 mM, 36-50 mM, 37-50 mM, 38-50 mM, 39-50 mM, 40-50 mM, 41-50 mM, 42-50 mM, 43-50 mM, 44-50 mM, 45-50 mM, 46-50 mM, 47-50 mM, 48-50 mM, 49-50 mM, 0-75 mM, 1-75 mM, 2-75 mM, 3-75 mM, 4-75 mM, 5-75 mM, 6-75 mM, 7-75 mM, 8-75 mM, 9-75 mM, 10-75 mM, 11-75 mM, 12-75 mM, 13-75 mM, 14-75 mM, 15-75 mM, 16-75 mM, 17-75 mM, 18-75 mM, 19-75 mM, 20-75 mM, 21-75 mM, 22-75 mM, 23-75 mM, 24-75 mM, 25-75 mM, 26-75 mM, 27-75 mM, 28-75 mM, 29-75 ITEM, 30-75 mM, 31-75 mM, 32-75 mM, 33-75 mM, 34-75 mM, 35-75 mM, 36-75 mM, 37-75 mM, 38-75 mM, 39-75 mM, 40-75 mM, 41-75 mM, 42-75 mM, 43-75 mM, 44-75 mM, 45-75 mM, 46-75 mM, 47-75 mM, 48-75 mM, 49-75 mM, 50-75 mM, 51-75 mM, 52-75 mM, 53-75 mM, 54-75 mM, 55-75 mM, 56-75 mM, 57-75 mM, 58-75 mM, 59-75 mM, 60-75 mM, 61-75 mM, 62-75 mM, 63-75 mM, 64-75 mM, 65-75 mM, 66-75 mM, 67-75 mM, 68-75 mM, 69-75 mM, 70-75 mM, 71-75 mM, 72-75 mM, 73-75 mM, 74-75 mM, 50-100 mM, 60-100 mM, 75-100 mM, 80-100 mM, or 90-100 mM.

In certain embodiments, the formulation may comprise 0-75 mM of magnesium chloride.

In certain embodiments, the formulation may comprise 0-5 mM of magnesium chloride.

In certain embodiments, the formulation may comprise 50-100 mM of magnesium chloride.

In certain embodiments, the formulation may comprise 2 mM of magnesium chloride.

In certain embodiments, the formulation may comprise 75 mM of magnesium chloride.

### Tris

In certain embodiments, at least one of the components in the formulation is Tris (also called tris(hydroxymethyl)aminomethane, tromethamine or THAM).

In certain embodiments, the concentration of Tris in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 0.4 mM, 10.5 mM, 10.6 mM, 10.7 mM, 10.8 mM, 10.9 mM, 11 mM, 11.1 mM, 11.2mM, 1.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM, or 15 mM.

The formulation may comprise Tris in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM, 9.6-10.1 mM 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In certain embodiments, the formulation may comprise 0-10 mM of Iris.

In certain embodiments, the formulation may comprise 2-12 mM of Tris.

In certain embodiments, the formulation may comprise 10 mM of Tris.

### Histidine

In certain embodiments, at least one of the components in the formulation is Histidine.

In certain embodiments, the concentration of Histidine in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1,7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5,6 mM, 5,7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may comprise Histidine in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3,5-4 mM, 3.6-4.1 mM, 3.7-4.2 mM, 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM- 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12.1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In certain embodiments, the formulation may comprise 0-10 mM of Histidine.

In certain embodiments, the formulation may comprise 2-12 mM of Histidine.

In certain embodiments, the formulation may comprise 10 mM of Histidine.

### Arginine

In certain embodiments, at least one of the components in the formulation is arginine.

In certain embodiments, the concentration of arginine may be, but is not limited to, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, or 100 mM.

The formulation may comprise arginine in a range of 0-5 mM, 1-5 mM, 2-5 mM, 3-5 mM, 4-5 mM, 0-10 mM, 1-10 mM, 2-10 mM, 3-10 mM, 4-10 mM, 5-10 mM, 6-10 mM, 7-10 mM, 8-10 mM, 9-10 mM, 0-25 mM, 1-25 mM, 2-25 mM, 3-25 mM, 4-25 mM, 5-25 mM, 6-25 mM, 7-25 mM, 8-25 mM, 9-25 mM, 10-25 mM, 11-25 mM, 12-25 mM, 13-25 mM, 14-25 mM, 15-25 mM, 16-25 mM, 17-25 mM, 18-25 mM, 19-25 mM, 20-25 mM, 21-25 mM, 22-25 mM, 23-25 mM, 24-25 mM, 0-50 mM, 1-50 mM, 2-50 mM, 3-50 mM, 4-50 mM, 5-50 mM, 6-50 mM, 7-50 mM, 8-50 mM, 9-50 mM, 10-50 mM, 11-50 mM, 12-50 mM, 13-50 mM, 14-50 mM, 15-50 mM, 16-50 mM, 17-50 mM, 18-50 mM, 19-50 mM, 20-50 mM, 21-50 mM, 22-50 mM, 23-50 mM, 24-50 mM, 25-50 mM, 26-50 mM, 27-50 mM, 28-29-50 mM, 30-50 mM, 31-50 mM, 32-50 mM, 33-50 mM, 34-50 mM, 35-50 mM, 36-50 mM, 37-50 mM, 38-50 mM, 39-50 mM, 40-50 mM, 41-50 mM, 42-50 mM, 43-50 mM, 44-50 mM, 45-50 mM, 46-50 mM, 47-50 mM, 48-50 mM, 49-50 mM, 0-75 mM, 1-75 mM, 2-75 mM, 3-75 mM, 4-75 mM, 5-75 mM, 6-75 mM, 7-75 mM, 8-75 mM, 9-75 mM, 10-11-75 mM, 12-75 mM, 13-75 mM, 14-75 mM, 15-75 mM, 16-75 mM, 17-75 mM, 18-75 mM, 19-75 mM, 20-75 mM, 21-75 mM, 22-75 mM, 23-75 mM, 24-75 mM, 25-75 mM, 26-75 mM, 27-75 mM, 28-75 mM, 29-75 mM, 30-75 ITEM, 31-75 mM, 32-75 mM, 33-75 mM, 34-75 mM, 35-75 mM, 36-75 mM, 37-75 mM, 38-75 mM, 39-75 mM, 40-75 mM, 41-75 mM, 42-75 mM, 43-75 mM, 44-75 mM, 45-75 mM, 46-75 mM, 47-75 mM, 48-75 mM, 49-75 mM, 50-75 mM, 51-75 mM, 52-75 mM, 53-75 mM, 54-75 mM, 55-75 mM, 56-75 mM, 57-75 mM, 58-75 mM, 59-75 mM, 60-75 mM, 61-75 mM, 62-75 mM, 63-75 mM, 64-75 mM, 65-75 mM, 66-75 mM, 67-75 mM, 68-75 mM, 69-75 mM, 70-75 mM, 71-75 mM, 72-75 mM, 73-75 mM, 74-75 mM, 50-100 mM, 60-100 mM, 75-100 mM, 80-100 mM, or 90-100 mM.

In certain embodiments, the formulation may comprise 0-75 mM, of arginine.

In certain embodiments, the formulation may comprise 50-100 mM of arginine.

In certain embodiments, the formulation may comprise 75 mM of arginine.

### Hydrochloric Acid

In certain embodiments, at least one of the components in the formulation is hydrochloric acid.

In certain embodiments, the concentration of hydrochloric acid in a formulation may be, but is not limited to, 0.1 mM, 0.2 mM, 0.3 mM, 0.4 mM, 0.5 mM, 0.6 mM, 0.7 mM, 0.8 mM, 0.9 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12,3 mM, 12.4 m12.4 mM, 12.5 mM, 12.6 mM, 12.7 mM, 12.8 mM, 12.9 mM, 13 mM, 13.1 mM, 13.2 mM, 13,3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM or 15 mM.

The formulation may comprise hydrochloric acid in a range of 0-0.5 mM, 0.1-0.6 mM, 0.2-0.7 mM, 0.3-0.8 mM, 0.4-0.9 mM, 0.5-1 mM, 0.6-1.1 mM, 0.7-1.2 mM, 0.8-1.3 mM, 0.9-1.4 mM, 1-1.5 mM, 1.1-1.6 mM, 1.2-1.7 mM, 1.3-1.8 mM, 1.4-1.9 mM, 1.5-2 mM, 1.6-2.1 mM, 1.7-2.2 mM, 1.8-2.3 mM, 1.9-2.4 mM, 2-2.5 mM, 2.1-2.6 mM, 2.2-2.7 mM, 2.3-2.8 mM, 2.4-2.9 mM, 2.5-3 mM, 2.6-3.1 mM, 2.7-3.2 mM, 2.8-3.3 mM, 2.9-3.4 mM, 3-3.5 mM, 3.1-3.6 mM, 3.2-3.7 mM, 3.3-3.8 mM, 3.4-3.9 mM, 3.5-4 mM, 3.6-4.1 mM, 3.7-4.2 mM 3.8-4.3 mM, 3.9-4.4 mM, 4-4.5 mM, 4.1-4.6 mM, 4.2-4.7 mM, 4.3-4.8 mM, 4.4-4.9 mM, 4.5-5 mM, 4.6-5.1 mM, 4.7-5.2 mM, 4.8-5.3 mM, 4.9-5.4 mM, 5-5.5 mM, 5.1-5.6 mM, 5.2-5.7 mM, 5.3-5.8 mM, 5.4-5.9 mM, 5.5-6 mM, 5.6-6.1 mM, 5.7-6.2 mM, 5.8-6.3 mM, 5.9-6.4 mM, 6-6.5 mM, 6.1-6.6 mM, 6.2-6.7 mM, 6.3-6.8 mM, 6.4-6.9 mM, 6.5-7 mM, 6.6-7.1 mM, 6.7-7.2 mM, 6.8-7.3 mM, 6.9-7.4 mM, 7-7.5 mM, 7.1-7.6 mM, 7.2-7.7 mM, 7.3-7.8 mM, 7.4-7.9 mM, 7.5-8 mM, 7.6-8.1 mM, 7.7-8.2 mM, 7.8-8.3 mM, 7.9-8.4 mM, 8-8.5 mM, 8.1-8.6 mM, 8.2-8.7 mM, 8.3-8.8 mM, 8.4-8.9 mM, 8.5-9 mM, 8.6-9.1 mM, 8.7-9.2 mM, 8.8-9.3 mM, 8.9-9.4 mM, 9-9.5 mM, 9.1-9.6 mM, 9.2-9.7 mM, 9.3-9.8 mM, 9.4-9.9 mM, 9.5-10 mM 9.6-10.1 mM, 9.7-10.2 mM, 9.8-10.3 mM, 9.9-10.4 mM, 10-10.5 mM, 10.1-10.6 mM, 10.2-10.7 mM, 10.3-10.8 mM, 10.4-10.9 mM, 10.5-11 mM, 10.6-11.1 mM, 10.7-11.2 mM, 10.8-11.3 mM, 10.9-11.4 mM, 11-11.5 mM, 11.1-11.6 mM, 11.2-11.7 mM, 11.3-11.8 mM, 11.4-11.9 mM, 11.5-12 mM, 11.6-12.1 mM, 11.7-12.2 mM, 11.8-12.3 mM, 11.9-12.4 mM, 12-12.5 mM, 12,1-12.6 mM, 12.2-12.7 mM, 12.3-12.8 mM, 12.4-12.9 mM, 12.5-13 mM, 12.6-13.1 mM, 12.7-13.2 mM, 12.8-13.3 mM, 12.9-13.4 mM, 13-13.5 mM, 13.1-13.6 mM, 13.2-13.7 mM, 13.3-13.8 mM, 13.4-13.9 mM, 13.5-14 mM, 13.6-14.1 mM, 13.7-14.2 mM, 13.8-14.3 mM, 13.9-14.4 mM, 14-14.5 mM, 14.1-14.6 mM, 14.2-14.7 mM, 14.3-14.8 mM, 14.4-14.9 mM, 14.5-15 mM, 0-1 mM, 1-2 mM, 2-3 mM, 3-4 mM, 4-5 mM, 5-6 mM, 6-7 mM, 7-8 mM, 8-9 mM, 9-10 mM, 10-11 mM, 11-12 mM, 12-13 mM, 13-14 mM, 14-15 mM, 15-16 mM, 0-2 mM, 1-3 mM, 2-4 mM, 3-5 mM, 4-6 mM, 5-7 mM, 6-8 mM, 7-9 mM, 8-10 mM, 9-11 mM, 10-12 mM, 11-13 mM, 12-14 mM, 13-15 mM, 0-3 mM, 1-4 mM, 2-5 mM, 3-6 mM, 4-7 mM, 5-8 mM, 6-9 mM, 7-10 mM, 8-11 mM, 9-12 mM, 10-13 mM, 11-14 mM, 12-15 mM, 0-4 mM, 1-5 mM, 2-6 mM, 3-7 mM, 4-8 mM, 5-9 mM, 6-10 mM, 7-11 mM, 8-12 mM, 9-13 mM, 10-14 mM, 11-15 mM, 0-5 mM, 1-6 mM, 2-7 mM, 3-8 mM, 4-9 mM, 5-10 mM, 6-11 mM, 7-12 mM, 8-13 mM, 9-14 mM, 10-15 mM, 0-6 mM, 1-7 mM, 2-8 mM, 3-9 mM, 4-10 mM, 5-11 mM, 6-12 mM, 7-13 mM, 8-14 mM, 9-15 mM, 0-7 mM, 1-8 mM, 2-9 mM, 3-10 mM, 4-11 mM, 5-12 mM, 6-13 mM, 7-14 mM, 8-15 mM, 0-8 mM, 1-9 mM, 2-10 mM, 3-11 mM, 4-12 mM, 5-13 mM, 6-14 mM, 7-15 mM, 0-9 mM, 1-10 mM, 2-11 mM, 3-12 mM, 4-13 mM, 5-14 mM, 6-15 mM, 0-10 mM, 1-11 mM, 2-12 mM, 3-13 mM, 4-14 mM, 5-15 mM, 0-11 mM, 1-12 mM, 2-13 mM, 3-14 mM, 4-15 mM, 0-12 mM, 1-13 mM, 2-14 mM, 3-15 mM, 0-13 mM, 1-14 mM, 2-15 mM, 0-14 mM, 1-15 mM, or 0-15 mM.

In certain embodiments, the formulation may comprise 0-10 mM of hydrochloric acid.

In certain embodiments, the formulation may comprise 6.2-6.3 mM of hydrochloric acid.

In certain embodiments, the formulation may comprise 8.9-9 mM of hydrochloric acid.

In certain embodiments, the formulation may comprise 6.2 mM of hydrochloric acid.

In certain embodiments, the formulation may comprise 6.3 mM of hydrochloric acid.

In certain embodiments, the formulation may comprise 8.9 mM of hydrochloric acid.

In certain embodiments, the formulation may comprise 9 mM of hydrochloric acid.

### Sugar

In certain embodiments, the formulation may comprise at least one sugar and/or sugar substitute.

In certain embodiments, the formulation may comprise at least one sugar and/or sugar substitute to increase the stability of the formulation. This increase in stability may provide longer hold times for in-process pools, provide a longer “shelf-life”, increase the concentration of AAV particles in solution (e.g., the formulation is able to have higher concentrations of AAV particles without rAAV dropping out of the solution) and/or reduce the generation or formation of aggregation in the formulations.

In certain embodiments, the inclusion of at least one sugar and/or similar substitute in the formulation may increase the stability of the formulation by 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%, 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85% 0-90%, 10-95%, 15-25%, 15-30%, 15-35%, 15-40%, 15-45%, 15-50%, 15-55%, 15-60% 5-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 75-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as compared to the same formulation without the sugar and/or sugar substitute.

In certain embodiments, the sugar and/or sugar substitute is used in combination with a phosphate buffer for increased stability. The combination of the sugar and/or sugar substitute with the phosphate butter may increase stability by 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%, 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 75-85%, 75-90%, 75-95%, 30-40%, 30-45%, 30-50%, 55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%.45-60%. 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, -80%, 70-85%, 70-90%, 70-95%, 75-85%, 75-90%, 75-95%, 80-90%, 80-95%, or 90-95% as compared to the same formulation without the sugar and/or sugar substitute. As a non-limiting example, the sugar is sucrose. As another non-limiting example, the sugar is trehalose. As another non-limiting, example, the sugar substitute is sorbitol.

In certain embodiments, the hold time of the formulation may be increased by 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%, 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as compared to the same formulation without the sugar and/or sugar substitute.

In certain embodiments, the shelf-life of the formulation may be increased by 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%. 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, -5-.?5,©, 25-40%, 25-45%, 25-50%, 25-55%, 25-60%, 25-65%, 75-70%, 75-75%, 75-80%, 75-85%, 75-90%, 75-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as compared to the same formulation without the sugar and/or sugar substitute. The shelf-life may he 1. 2, 3, 4, 5, 6, 7, 8, 9, 10, 11, 12, 13, 14, 15, 16, 17, 18, 19, 20, 21, 22, 23, 24 hours, or 1, 2, 3, 4 weeks, or 1, 2, 3, 4, 5, 6, 7, 8, 9, 10, 11, 12, 13, 14, 15, 16, 17, 18, 19, 20, 21, 22, 23, 24 months, or 1, 2, 3, 4, 5, 6, 7 or more than 7 years.

In certain embodiments, the concentration of the AAV particles in the formulation may be increased by 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or more than 95%, 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 0-75%, 0-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 2035%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, ?5-75%, ?5-80%, ?5-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as compared to the same formulation without the sugar and/or sugar substitute.

In certain embodiments, as a result of the addition of a sugar and/or sugar substitute, the formulation generation of aggregates in the formulation may be reduced by 1%, 2%, 3%, 4%, 5%, 10%, 15%, 20%, 25%, 30%, 35%, 40%, 45%, 50%, 55° ,4,, 60%, 65%, 700o, 75%, 80%, 85%, 90%, 95%, or more than 95%, 1-5%, 545%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80⁰o, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60⁰. 45-65%, 45-70%, 45-75%, 45-80%, 45-85%, 45-90% 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as compared to the same formulation without the sugar and/or sugar substitute.

In certain embodiments, as a result of the addition of a sugar and/or sugar substitute, the formulation or generation of aggregates may be 1%, 2%, 3%, 4%, 5%, 10%, 15%, 20%, 25%, 30%, 350, 40%, 45%, 50%, 55%, 60%, 65%, 70%, 75%, 80%, 85%, 90%. 95%, or more than 95%, 1-5%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80?/0, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85⁰, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95% as determined by a method known in the art (e.g., by SLS measurement) and as compared to the same formulation without the sugar and/or sugar substitute. As a non-limiting example, the aggregation of a formulation can be less than 2% by the addition of at least one sugar and/or sugar substitute to the formulation. Additional aggregates can be removed by methods known in the art.

In certain embodiments, the formulation may comprise a sugar arid/or sugar substitute at 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, or 10% w/v.

In certain embodiments, the formulation may comprise a sugar and/or sugar substitute in a range of 0-1%, 0.1-1%, 0.2-1%, 0.3-1%, 0.4-1%, 0.5-1%, 0.6-1%, 0.7-1%, 0.8-1%, 0.9-1%, 0-1.5%, 0.1-1.5%, 0.2-1.5%, 0.3-1.5%, 0.4-1.5%, 0.5-1.5%, 0.6-1.5%, 0.7-1.5%, 0.8-1.5%, 0.9-1.5%, 1-1.5%, 1.1-1.5%, 1.2-1.5%, 1.3-1.5%, 1.4-1.5%, 0-2%, 0.1-2%, 0.2-2%, 0.3-2%, 0.4-2%, 0.5-2%, 0.6-2%, 0.7-2%, 0.8-2%, 0.9-2%, 1-2%, 1.1-2%, 1.2-2%, 1.3-2%, 1.4-2%, 1.5-2%, 1.6-2%, 1.7-2%, 1.8-2%, 1.9-2%, 0-2.5%, 0.1-2.5%, 0.2-2.5%, 0.3-2.5%, 0.4-2.5% 0.5-2.5%, 0.6-2.5%, 0.7-2.5%, 0.8-2.5%, 0.9-2.5%, 1-2.5%, 1.1-2.5%, 1.2-2.5%, 1.3-2.5%, 1.4-2.5%, 1.5-2.5%, 1.6-2.5%, 1.7-2.5%, 1.8-2.5%, 19-2.5%, 2-2.5%, 2.1-2.5%, 2.2-2.5%, 2.3-2.5%, 2.4-2.5%, 0-3%, 0.1-3%, 0.2-3%, 0.3-3%, 0.4-3%, 0.5-3%, 0.6-3%, 0.7-3%, 0.8-3%, 0.9-3%, 1-3%, 1.1-3%, 1.2-3%, 1.3-3%, 1.4-3%, 1.5-3%, 1.6-3%, 1.7-3%, 1.8-3%, 1.9-3%, 2-3%, 2.1-3%, 2.2-3%, 2.3-3%, 2.4-3%, 2.5-3%, 2.6-3%, 2.7-3%, 2.8-3%, 2.9-3%, 0-3.5%, 0.1-3.5%, 0.2-3.5%, 0.3-3.5%, 0.4-3.5%, 0.5-3.5%, 0.6-3.5%, 0.7-3.5%, 0.8-3.5%, 0.9-3.5%, 1-3.5%, 1.1-3.5%, 1.2-3.5%, 1.3-3.5%, 1.4-3.5%, 1.5-3.5%, 1.6-3.5%, 1.7-3.5%, 1.8-3.5%, 1.9-3.5%, 2-3.5%, 2.1-3.5%, 2.2-3.5%, 2.3-3.5%, 2.4-3.5%, 2.5-3.5%, 2.6-3.5%, 2.7-3.5%, 2.8-3.5%, 2.9-3.5%, 3-3.5%, 3,1-3,5%, 3.2-3,5%, 3.3-3.5%, 3.4-3.5%, 0-4%, 0.1-4%, 0.2-4%, 0.3-4%, 0.4-4%, 0.5-4%, 0.6-4%, 0.7-4%, 0.8-4%, 0.9-4%, 1-4%, 1.1-4%, 1.2-4%, 1.3-4%, 1.4-4%, 1.5-4%, 1.6-4%, 1.7-4%, 1.8-4%, 19-4%, 2-4%, 2.1-4%, 2.2-4%, 2.3-4%, 2.4-4%, 2.5-4%, 2.6-4%, 2.7-4%, 2.8-4%, 2.9-4%, 3-4%, 3.1-4%, 3.2-4%, 3.3-4%, 3.4-4%, 3.5-4%, 3.6-4%, 3.7-4%, 3.8-4%, 3.9-4%, 0-4.5%, 0.1-4.5%, 0.2-4.5%, 0.3-4.5%, 0.4-4.5%, 0.5-4.5%, 0.6-4.5%, 0.7-4.5%, 0.8-4.5%, 0,9-4,5%, 1-4.5%, 1.1-4.5%, 1.2-4.5%, 1.3-4.5%, 1.4-4.5%, 1.5-4.5%, 1.6-4.5%, 1.7-4.5%, 1.8-4.5%, 1.9-4.5%, 2-4.5%, 2.1-4.5%, 2.2-4.5%, 2.3-4.5%, 2.4-4.5%, 2.5-4.5%, 2.6-4.5%, 2.7-4.5%, 2.8-4.5%, 2.9-4.5%, 3-4.5%, 3.1-4.5%, 3.2-4.5%, 3.3-4.5%, 3.4-4.5%, 3.5-4.5%, 3.6-4.5%, 3.7-4.5%, 3.8-4.5%, 3.9-4.5%, 4-4.5%, 4.1-4.5%, 4.2-4.5%, 4.3-4.5%, 4.4-4.5%, 0-5%, 0.1-5%, 0.2-5%, 0.3-5%, 0.4-5%, 0.5-5%, 0.6-5%, 0.7-5%, 0.8-5%, 0.9-5%, 1-5%, 1.1-5%, 1.2-5%, 1.3-5%, 1.4-5%, 1.5-5%, 1.6-5%, 1.7-5%, 1.8-5%, 1.9-5%, 2-5%, 2.1-5%, 2.2-5%, 2.3-5%, 2.4-5%, 2.5-5%, 2.6-5%, 2.7-5%, 2.8-5%, 2.9-5%, 3-5%, 3.1-5%, 3,2-5%, 3.3-5%, 3,4-5%, 3.5-5%, 3.6-5%, 3.7-5%, 3.8-5%, 3.9-5%, 4-5%, 4.1-5%, 4.2-5%, 4.3-5%, 4.4-5%, 4.5-5%, 4.6-5%, 4.7-5%, 4.8-5%, 4.9-5%, 0-5.5%, 0.1-5.5%, 0.2-5.5%, 0.3-5.5%, 0.4-5.5%, 0.5-5.5%, 0.6-5.5%, 0.7-5.5%, 0.8-5.5%, 0.9-5.5%, 1-5.5%, 1.1-5.5%, 1.2-5.5%, 1.3-5.5%, 1.4-5.5%, 1.5-5.5%, 1.6-5.5%, 1.7-5.5%, 1.8-5.5%, 1.9-5.5%, 2-5,5%, 2.1-5.5%, 2.2-5.5%, 2.3-5.5%, 2.4-5.5%, 2.5-5.5%, 2.6-5.5%, 2.7-5.5%, 2.8-5.5%, 2.9-5.5%, 3-5.5%, 3.1-5.5%, 3.2-5.5%, 3.3-5.5%, 3.4-5.5%, 3.5-5.5%, 3.6-5.5%, 3.7-5.5%, 3.8-5.5%, 3.9-5.5%, 4-5.5%, 4.1-5.5%, 4.2-5.5%, 4.3-5.5%, 4.4-5.5%, 4.5-5.5%, 4.6-5.5%, 4.7-5.5%, 4.8-5.5%, 4.9-5.5%, 5-5.5%, 5.1-5.5%, 5.2-5.5%, 5.3-5.5%, 5.4-5.5%, 0-6%, 0.1-6%, 0.2-6%, 0.3-6%, 0.4-6%, 0.5-6%, 0.6-6%, 0.7-6%, 0.8-6%, 0.9-6%, 1-6%, 1.1-6%, 1.2-6%, 1.3-6%, 1.4-6%, 1.5-6%, 1.6-6%, 1.7-6%, 1.8-6%, 1.9-6%, 2-6%, 2.1-6%, 2.2-6%, 2.3-6%, 2.4-6%, 2.5-6%, 2.6-6%, 2.7-6%, 2.8-6%, 2.9-6%, 3-6%, 3.1-6%, 3.2-6%, 3.3-6%, 3.4-6%, 3.5-6%, 3.6-6%, 3.7-6%, 3.8-6%, 3.9-6%, 4-6%, 4.1-6%, 4.2-6%, 4.3-6%, 4.4-6%, 4.5-6%, 4.6-6%, 4.7-6%, 4.8-6%, 4.9-6%, 5-6%, 5.1-6%, 5.2-6%, 5.3-6%, 5.4-6%, 5.5-6%, 5.6-6%, 5.7-6%, 5.8-6%) 5.9-6%, 0-6.5%, 0.1-6.5%, 0.2-6.5%, 0.3-6.5%, 0.4-6.5%, 0.5-6.5%, 0.6-6.5%, 0.7-6.5%, 0.8-6.5%, 0.9-6.5%, 1-6.5%, 1.1-6.5%, 1.2-6.5%, 1.3-6.5%, 1.4-6.5%, 1.5-6.5%, 1.6-6.5%, 1.7-6.5%, 1,8-65%, 1,9-6,5%, 2-6.5%, 2.1-6.5%, 2.2-6.5%, 2.3-6.5%, 2.4-6.5%, 2.5-6.5%. 2.6-6.5%, 2.7-6.5%, 2.8-6.5%, 2.9-6.5%, 3-6.5%, 3.1-6.5%, 3.2-6.5%, 3.3-6.5%, 3.4-6.5%, 3.5-6.5%, 3.6-6.5%, 3.7-6.5%, 3.8-6.5%, 3.9-6.5%, 4-6.5%, 4.1-6.5%, 4.2-6.5%, 4.3-6.5%, 4.4-6.5%, 4.5-6.5%, 4.6-6.5%, 4.7-6.5%, 4.8-6.5%, 4.9-6.5%, 5-6.5%, 5.1-6.5%, 5.2-6.5%, 5.3-6.5%, 5.4-6.5%, 5.5-6.5%, 5.6-6.5%, 5.7-6.5%, 5.8-6.5%, 5.9-6.5%, 6-6.5%, 6.1-6.5%, 6.2-6.5%, 6.3-6.5%, 6.4-6.5%, 0-7%, 0.1-7%, 0.2-7%, 0.3-7%, 0.4-7%, 0.5-7%, 0.6-7%, 0.7-7%, 0.8-7%, 0.9-7%, 1-7%, 1.1-7%, 1.2-7%, 1.3-7%, 1,4-7%, 1.5-7%, 1.6-7%, 1.7-7%, 1.8-7%, 19-7%, 2-7%, 2.1-7%, 2,2-7%, 2.3-7%, 2.4-7%, 2.5-7%, 2.6-7%, 2.7-7%, 2.8-7%, 2.9-7%, 3-7%, 3.1-7%, 3,2-7%, 3.3-7%, 3.4-7%, 3.5-7%, 3.6-7%, 3.7-7%, 3.8-7%, 3.9-7%, 4-7%, 4.1-7%, 4.2-7%, 4.3-7%, 4.4-7%, 4.5-7%, 4.6-7%, 4.7-7%, 4.8-7%, 4.9-7%, 5-7%, 5,1-7%, 5.2-7%, 5,3-7%, 5.4-7%, 5.5-7%, 5.6-7%, 5.7-7%, 5.8-7%, 5.9-7%, 6-7%, 6.1-7%, 6,2-7%, 6.3-7%, 6,4-7%, 6.5-7%, 6.6-7%, 6.7-7%, 6.8-7%, 6.9-7%, 0-7.5%, 0.1-7.5%, 0.2-7.5%, 0.3-7.5%, 0.4-7.5%, 0.5-7.5%, 0.6-7.5%, 0.7-7.5%, 0.8-7.5%, 0.9-7.5%, 1-7.5%, 1.1-7.5%, 1.2-7.5%, 1.3-7.5%, 1.4-7.5%, 0.5-7.5%, 1.6-7.5%, 1.7-7.5%, 1.8-7.5%, 1.9-7.5%, 2-7.5%, 2.1-7.5%, 2.2-7.5%, 2.3-7.5%, 2.4-7.5%, 2.5-7.5%, 2.6-7.3%, 2.7-7.5%, 2.8-7.5%, 2,9-7,5%, 3-7.5%, 3.1-7.5%, 3.2-7.5%, 3.3-7.5%, 3.4-7.5%, 3.5-7.5%, 3.6-7.5%, 3.7-7.5%, 3.8-7.5%, 3.9-7.5%, 4-7.5%, 4.1-7.5%, 4.2-7.5%, 4.3-7.5%, 4.4-7.5%, 4.5-7.5%, 4.6-7.5%, 4.7-7.5%, 4.8-7.5%, 4.9-7.5%, 5-7.5%, 5.1-7.5%, 5.2-7.5%, 5.3-7.5%, 5.4-7.5%, 5.5-7.5%, 5.6-7.5%, 5.7-7.5%, 5.8-7.5%, 5.9-7.5%, 6-7.5%, 6.1-7.5%, 6.2-7.5%, 6.3-7.5%, 6.4-7.5%, 6.5-7.5%, 6.6-7.5%, 6.7-7.5%, 6.8-7.5%, 6.9-7.5%, 7-7.5%, 7.1-7.5%, 7.2-7.5%, 7.3-7.5%, 7.4-7.5%, 0-8%, 0.1-8%, 0.2-8%, 0.3-8%, 0.4-8%, 0.5-8%, 0.6-8%, 0.7-8%, 0.8-8%, 0.9-8%, 1-8%, 1.1-8%, 1.2-8%, 1.3-8%, 1.4-8%, 1.5-8%, 1.6-8%, 1.7-8%, 1.8-8%, 1.9-8%, 2-8%, 2.1-8%, 2.2-8%, 2.3-8%, 2.4-8%, 2.5-8%, 2.6-8%, 2.7-8%, 2.8-8%, 2.9-8%, 3-8%, 3.1-8%, 3.2-8%, 3.3-8%, 3.4-8%, 3.5-8%, 3.6-8%, 3.7-8%, 3.8-8%, 3.9-8%, 4-8%, 4.1-8%, 4.2-8%, 4.3-8%, 4.4-8%, 4.5-8%, 4.6-8%, 4.7-8%, 4.8-8%, 4.9-8%, 5-8%, 5,1-8%, 5.2-8%, 5,3-8%, 5.4-8%, 5.5-8%, 5.6-8%, 5.7-8%, 5.8-8%, 5.9-8%, 6-8%, 6.1-8%, 6.2-8%, 6.3-8%, 6.4-8%, 6.5-8%, 6.6-8%, 6.7-8%, 6.8-8%, 6.9-8%, 7-8%, 7.1-8%, 7.2-8%, 7.3-8%, 7.4-8%, 7.5-8%, 7.6-8%, 7.7-8%, 7.8-8%, 7.9-8%, 0-8.5%, 0.1-8.5%, 0.2-8.5%, 0.3-8.5%, 0.4-8.5%, 0.5-8,5%, 0.6-8.5%, 0.7-8.5%, 0.8-8.5%, 0.9-8.5%, 1-8.5%, 1.1-8.5%, 1.2-8.5%, 1.3-8.5%, 1,4-8,5%, 1.5-8.5%, 1.6-8.5%, 1.7-8.5%, 1.8-8.5%, 9-8.5%, 2-8.5%, 2.1-8.5%, 2.2-8.5%, 2.3-8.5%, 2.4-8.5%, 2.5-8.5%, 2.6-8.5%, 2.7-8.5%, 2.8-8.5%, 2.9-8.5%, 3-8.5%, 3.1-8.5%, 3.2-8.5%, 3.3-8.5%, 3.4-8.5%, 3.5-8.5%, 3.6-8.5%, 3.7-8.5%, 3.8-8.5%, 3.9-8.5%, 4-8.5%, 4.1-8.5%, 4.2-8.5%, 4.3-8.5%, 4.4-8.5%, 4.5-8.5%, 4.6-8.5%, 4.7-8.5%, 4.8-8.5%, 4.9-8.5%, 5-8.5%, 5.1-8,5%, 5.2-8.5%, 5.3-8.5%, 5.4-8.5%, 5.5-8.5%, 5.6-8.5%, 5.7-8.5%, 5.8-8.5%, 5.9-8.5%, 6-8.5%, 6.1-8.5%, 6.2-8.5%, 6.3-8.5%, 6.4-8.5%, 6.5-8.5%, 6.6-8.5%, 6.7-8.5%, 6.8-8,5%, 6.9-8.5%, 7-8.5%, 7.1-8.5%, 7.2-8,5%, 7.3-8.5%, 7.4-8.5%, 7.5-8.5%, 7.6-8.5%, 7,7-8,5%, 7.8-8.5%, 7.9-8.5%, 8-8.5%, 8,1-8,5%, 8.2-8.5%, 8.3-8.5%, 8.4-8.5%, 0-9%, 0.1-9%, 0.2-9%, 0.3-9%, 0.4-9%, 0.5-9%, 0.6-9%, 0.7-9%, 0.8-9%, 0.9-9%, 1-9%, 1.1-9%, 1.2-9%, 1.3-9%, 1.4-9%, 1.5-9%, 1.6-9%, 1.7-9%, 1.8-9%, 1.9-9%, 2-9%, 2.1-9%, 2.2-9%, 2.3-9%, 2.4-9%, 2.5-9%, 2.6-9%, 2.7-9%, 2.8-9%, 2.9-9%, 3-9%, 3.1-9%, 3.2-9%, 3.3-9%, 3.4-9%, 3.5-9%, 3,6-9%, 3.7-9%, 3.8-9%, 3.9-9%, 4-9%, 4.1-9%, 4.2-9%, 4.3-9%, 4.4-9%, 4.5-9%, 4.6-9%, 4.7-9%, 4.8-9%, 4.9-9%, 5-9%, 5.1-9%, 5.2-9%, 5.3-9%, 5.4-9%, 5.5-9%, 5.6-9%, 5.7-9%, 5.8-9%, 5.9-9%, 6-9%, 6.1-9%, 6.2-9%, 6.3-9%, 6.4-9%, 6.5-9%, 6.6-9%, 6.7-9%, 6.8-9%, 6.9-9%, 7-9%, 7.1-9%, 7.2-9%, 7.3-9%, 7.4-9%, 7.5-9%, 7.6-9%, 7.7-9%, 7.8-9%, 7.9-9%, 8-9%, 8.1-9%, 8.2-9%, 8.3-9%, 8.4-9%, 8.5-9%, 8.6-9%, 8.7-9%, 8.8-9%, 8.9-9%, 0-9.5%, 0.1.-9.5%, 0.2-9.5%, 0.3-9.5%, 0.4-9.5%, 0.5-9.5%, 0.6-9.5%, 0.7-9.5%, 0.8-9.5%, 0.9-9.5%, 1-9.5%, 1.1-9.5%, 1.2-9.5%, 1.3-9.5%, 0.4-9.5%, 1.5-9.5%, 1.6-9.5%, 1.7-9.5%, 1.8-9.5%, 1.9-9.5%, 2-9.5%, 2.1-9.5%, 2.2-9.5%, 2.3-9.5%, 2.4-9.5%, 2.5-9.5%, 2.6-9.5%, 2.7-9.5%, 2.8-9.5%, 2.9-9.5%, 3-9.5%, 3.1-9.5%, 3.2-9.5%, 3.3-9.5%, 3.4-9.5%, 3.5-9.5%, 3.6-9.5%, 3.7-9.5%, 3.8-9.5%, 3.9-9.5%, 4-9.5%, 4.1-9.5%, 4.2-9.5%, 4.3-9.5%, 4.4-9.5%, 4.5-9.5%, 4.6-9.5%, 4.7-9.5%, 4.8-9.5%, 4.9-9.5%, 5-9.5%, 5.1-9.5%, 5.2-9.5%, 5.3-9.5%, 5.4-9.5%, 5.5-9.5%, 5.6-9.5%, 5.7-9.5%, 5.8-9.5%, 5.9-9.5%, 6-9.5%, 6.1-9.5%, 6.2-9.5%, 6.3-9.5%, 6.4-9.5%, 6.5-9.5%, 6.6-9.5%, 6.7-9.5% 6.8-9.5%, 6.9-9.5%, 7-9.5%, 7.1-9.5%, 7.2-9.5%, 7.3-9.5%, 7.4-9.5%, 7.5-9.5%, 7.6-9.5%, 7.7-9.5%, 7.8-9.5%, 7.9-9.5%, 8-9.5%, 8.1-9.5%, 8.2-9.5%, 8.3-9.5%, 8.4-9.5%, 8.5-9.5%, 8.6-9.5%, 8.7-9.5%, 8.8-9.5%, 8.9-9.5%, 9-9.5%, 9.1-9.5%, 9.2-9.5%, 9.3-9.5%, 9.4-9.5%, 0-10%, 0.1-10%, 0.2-10%, 0.3-10%, 0.4-10%, 0.5-10%, 0.6-10%, 0.7-10%, 0.8-10%, 0.9-10%, 1-10%, 1.1-10%, 1.2-10%, 1.3-10%, 1.4-10%, 1.5-10%, 1.6-10%, 1.7-10%, 1.8-10%, 1.9-10%, 2-10%, 2.1-10%, 2.2-10%, 2.3-10%, 2.4-10%, 2.5-10%, 2.6-10%, 2.7-10%, 2.8-10%, 2.9-10%, 3-10%, 3.1-10%, 3.2-10%, 3.3-10%, 3.4-10%, 3.5-10%, 3.6-10%, 3.7-10%, 3.8-10%, 3.9-10%, 4-10%, 4.1-10%, 4.2-10%, 4.3-10% 44-10%, 4.5-10%, 4.6-10%, 4.7-10%, 4.8-10%, 1.9-10%, 5-10%, 5.1-10%, 5.2-10%, 5.3-10%, 5.4-10%, 5.5-10%, 5.6-10%, 5.7-1.0%, 5.8-10%, 5.9-10%, 6-10%, 6.1-10%, 6.2-10%, 6.3-10%, 6.4-10%, 6.5-10%, 6.6-10%, 6.7-10%, 6.8-10%, 6.9-10%, 7-10%, 7.1-10%, 7.2-10%, 7.3-10%, 7.4-10%, 7.5-10%, 7.6-10%, 7.7-10%, 7.8-10%, 7.9-10%, 8-10%, 8.1-10%, 8.2-10%, 8.3-10%, 8.4-10%, 8.5-10%, 8.6-10%, 8.7-10%, 8.8-10%, 8.9-10%, 9-10%, 9.1-10%, 9.2-10%, 9.3-10%, 9.4-10%, 9.5-10%, 9.6-10%, 9.7-10%, 9.8-10%, or 9.9-10%.

In certain embodiments, the formulation may comprise 0-10% w/v of a sugar and/or sugar substitute.

In certain embodiments, the formulation may comprise 0-9% w/v of a sugar and/or sugar substitute.

In certain embodiments, the formulation may comprise 1% w/v of a sugar and/or sugar substitute.

In certain embodiments, the formulation may comprise 2% w/v of a sugar and/or sugar substitute.

In certain embodiments, the formulation may comprise 3% w/v of a sugar and/or sugar substitute.

In certain embodiments, the formulation may comprise 4% w/v of a sugar and/or sugar substitute.

In certain embodiments, the formulation may comprise 5% w/v of a sugar and/or sugar substitute.

In certain embodiments, the formulation may comprise 6% w/v of a sugar and/or sugar substitute.

In certain embodiments, the formulation may comprise 7% w/v of a sugar and/or sugar substitute.

In certain embodiments, the formulation may comprise 8% w/v of a sugar and/or sugar substitute.

In certain embodiments, the formulation may comprise 9% w/v of a sugar and/or sugar substitute.

In certain embodiments, the formulation may comprise 10% w/v of a sugar and/or sugar substitute.

In certain embodiments, formulations of pharmaceutical compositions described herein may comprise a disaccharide. Suitable disaccharides that may be used in the formulation described herein may comprise sucrose, lactulose, lactose, maltose, trehalose, cellobiose, chitobiose, kojibiose, nigerose, isomaltose, β,β-trehalose, α,β-trehalose, sophorose, laminaribiose, gentiobiose, turanose, maltulose, palatinose, gentiobiulose, mannobiose, melibiose, melibiulose, rutinose, rutinulose, and xylobiose. The concentration of disaccharide (w/v) used in the formulation may be between 1%-15%, for example, between 1%-5%, between 3%-6%, between 5%-8%, between 7%-10%, or between 10%-15%.

In certain embodiments, formulations of pharmaceutical compositions described herein may comprise a sugar alcohol. As a non-limiting example, the sugar alcohol that may be used in the formulation described herein may comprise sorbitol. The concentration of sugar alcohol (w/v) used in the formulation may be between 1%-15%, for example, between 1%-5%, between 3%-6%, between 5%-8%, between 7%-10%, or between 10%-15%.

### Sucrose

In certain embodiments, the formulation may comprise at least one sugar which is disaccharide such as, but not limited to, sucrose.

In certain embodiments, the formulation may comprise sucrose at 0.1%, 0.2%, 0.3%, 0.4%, 0.5%, 0.6%, 0.7%, 0.8%, 0.9%, 1%, 1.1%, 1.2%, 1,3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%,5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, or 10% w/v.

In certain embodiments, the formulation may comprise sucrose in a range of 0-1%, 0.1-1%, 0.2-1%, 0.3-1%, 0.4-1%, 0.5-1%, 0.6-1%, 0.7-1%, 0.8-1%, 0.9-1%, 0-1.5%, 0.1-1.5%, 0.2-1.5%, 0.3-1.5%, 0.4-1.5%, 0.5-1.5%, 0.6-1.5%, 0.7-1.5%, 0.8-1.5%, 0.9-1.5%, 1-1.5%, 1.1-1.5%, 1.2-1.5%, 1.3-1.5%, 1.4-1.5%, 0-2%, 0.1-2%, 0.2-2%, 0.3-2%, 0.4-2%, 0.5-2%, 0.6-2%, 0.7-2%, 0.8-2%, 0.9-2%, 1-2%, 1.1-2%, 1.2-2%, 1.3-2%, 1.4-2%, 1.5-2%, 1.6-2%, 1.7-2%, 1.8-2%, 1.9-2%, 0-2.5%, 0.1-2.5%, 0.2-2.5%, 0.3-2.5%, 0.4-2.5%, 0.5-2.5%, 0.6-2.5%, 0.7-2.5%, 0.8-2.5%, 0.9-2.5%, 1-2.5%, 1.1-2.5%, 1.2-2.5%, 1.3-2.5%, 1.4-2.5%, 1.5-2.5%, 1.6-2.5%, 1.7-2.5%, 1.8-2.5%. 1.9-2.5%, 2-2.5%, 2.1-2.5%, 2.2-2.5%, 2.3-2.5%, 2.4-2.5%, 0-3%, 0.1-3%, 0.2-3%, 0.3-3%, 0.4-3%, 0.5-3%, 0.6-3%, 0.7-3%, 0.8-3%, 0.9-3%, 1-3%, 1.1-3%, 1.2-3%, 1.3-3%, 1.4-3%, 1.5-3%, 1.6-3%, 1.7-3%, 1.8-3%, 1.9-3%, 2-3%, 2.1-3%, 2.2-3%, 2.3-3%, 2.4-3%, 2.5-3%, 2.6-3%, 2.7-3%, 2.8-3%, 2.9-3%, 0-3.5%, 0.1-3.5%, 0.2-3.5%, 0.3-3.5%, 0.4-3.5%, 0.5-3.5%, 0.6-3.5%, 0.7-3.5%, 0.8-3.5%, 0.9-3.5%, 1-3.5%, 1.1-3.5%, 1.2-3.5%, 1.3-3.5%, 1.4-3.5%, 1.5-3.5%, 1.6-3.5%, 1.7-3.5%, 1.8-3.5%, 1.9-3.5%, 2-3.5%, 2.1-3.5%, 2.2-3.5%, 2.3-3.5%, 2.4-3.5%, 2.5-3.5%, 2.6-3.5%, 2.7-3.5%, 2.8-3.5%, 2.9-3.5%, 3-3.5%, 3.1-3.5%, 3.2-3.5%, 3.3-3.5%, 3.4-3.5%, 0-4%, 0.1-4%, 0.2-4%, 0.3-4%, 0.4-4%, 0.5-4%, 0.6-4%, 0.7-4%, 0.8-4%, 0.9-4%, 1-4%, 1.1-4%, 1.2-4%, 1.3-4%, 1.4-4%, 1.5-4%, 1.6-4%, 1.7-4%, 1.8-4%, 1.9-4%, 2-4%, 2.1-4%, 2.2-4%, 2.3-4%, 2.4-4%, 2.5-4%, 2.6-4%, 2.7-4%, 2.8-4%, 2.9-4%, 3-4%, 3.1-4%, 3.2-4%, 3.3-4%, 3.4-4%, 3.5-4%, 3.6-4%, 3.7-4%, 3.8-4%, 3.9-4%, 0-4.5%, 0.1-45%, 0.2-4.5%, 0.3-4.5%, 0.4-4.5%, 0.5-4.5%, 0.6-4.5%, 0.7-4.5%, 0.8-4.5%, 0.9-4.5%, 1-4.5%, 1.1-4.5%, 1.2-4.5%, 1.3-4.5%, 1.4-4.5%, 1.5-4.5%, 1.6-4.3%, 1.7-4.5%, 1.8-4.5%, 1.9-4.5%, 2-4.5%, 2.1-4.5%, 2.2-4.5%, 2.3-4.5%, 2.4-4.5%, 2.5-4.5%, 2.6-4.5%, 2.7-4.5%, 2.8-4.5%, 2.9-4.5%, 3-4.5%, 3.1-4.5%, 3.2-4.5%, 3.3-4.5%, 3.4-4.5%, 3.5-4.5%, 3.6-4.5%, 3.7-4.5%, 3.8-4.5%, 3.9-4.5%, 4-4.5%, 4.1-4.5%, 4.2-4.5%, 4.3-4.5%, 4.4-4.5%, 0-5%, 0.1-5%, 0.2-5%, 0.3-5%, 0.4-5%, 0.5-5%, 0.6-5%, 0.7-5%, 0.8-5%, 0.9-5%, 1-5%, 1.1-5%, 1,2-5%, 1.3-5%, 1,4-5%, 1.5-5%, 1.6-5%, 1.7-5%, 1.8-5%, 1.9-5%, 2-5%, 2.1-5%, 2.2-5%, 2.3-5%, 2.4-5%, 2.5-5%, 2.6-5%, 2.7-5%, 2.8-5%, 2.9-5%, 3-5%, 3.1-5%, 3.2-5%, 3.3-5%, 3.4-5%, 3.5-5%, 3.6-5%, 3.7-5%, 3.8-5%, 3.9-5%, 4-5%, 4.1-5%, 4.2-5%, 4.3-5%, 4.4-5%, 4.5-5%, 4.6-5%, 4.7-5%, 4.8-5%, 4.9-5%, 0-5.5%, 0.1-5.5%, 0.2-5.5%, 0.3-5.5%, 0.4-5.5%, 0.5-5.5%, 0.6-5.5%, 0.7-5.5%, 0.8-5.5%, 0.9-5.5%, 1-5.5%, 1.1-5.5%, 1.2-5.5%, 1.3-5.5%, 1.4-5.5%, 1.5-5.5%, 1.6-5.5%, 1.7-5.5%, 1.8-5.5%, 1.9-5.5%, 2-5.5%, 2.1-5.5%, 2.2-5.5%, 2.3-5.5%, 2.4-5.5%, 2.5-5.5%, 2.6-5.5%, 2.7-5.5%, 2.8-5.5%, 2.9-5.5%, 3-5.5%, 3.1-5.5%, 3.2-5.5%, 3.3-5.5%, 3.4-5.5%, 3.5-5.5%, 3.6-5.5%, 3.7-5.5%, 3.8-5.5%, 3.9-5.5%, 4-5.5%, 4.1-5.5%, 4.2-5.5%, 4.3-5.5%, 4.4-5.5%, 4.5-5.5%, 4.6-5.5%, 4.7-5.5%, 4.8-5.5%, 4.9-5.5%, 5-5.5%, 5.1-5.5%, 5.2-5.5%, 5.3-5.5%, 5.4-5.5%, 0-6%, 0.1-6%, 0.2-6%, 0.3-6%, 0.4-6%, 0.5-6%, 0.6-6%, 0.7-6%, 0.8-6%, 0.9-6%, 1-6%, 1.1-6%, 1.2-6%, 1.3-6%, 1.4-6%, 1.5-6%, 1.6-6%, 1.7-6%, 1.8-6%, 1.9-6%, 2-6%, 2.1-6%, 2.2-6%, 2.3-6%, 2.4-6%, 2.5-6%, 2.6-6%, 2.7-6%, 2.8-6%, 2.9-6%, 3-6%, 3.1-6%, 3.2-6%, 3.3-6%, 3.4-6%, 3.5-6%, 3.6-6%, 3.7-6%, 3.8-6%, 3.9-6%, 4-6%, 4.1-6%, 4.2-6%, 4.3-6%, 4.4-6%, 4.5-6%, 4.6-6%, 4.7-6%, 4.8-6%, 4.9-6%, 5-6%, 5.1-6%, 5.2-6%, 5.3-6%, 5.4-6%, 5.5-6%, 5.6-6%, 5.7-6%, 5.8-6%, 5.9-6%, 0-6,5%, 01-65%, 0.2-6.5%, 0.3-6.5%, 0.4-6.5%, 0.5-6.5%, 0.6-6.5%, 0.7-6.5%, 0.8-6.5%, 0.9-6.5%, 1-6.5%, 1.1-6.5%, 1.2-6.5%, 1.3-6.5%, 1.4-6.5%, 1.5-6.5%, 1.6-6.5%, 1.7-6.5%, 1.8-6.5%, 1.9-6.5%, 2-6.5%, 2.1-6.5%, 2.2-6.5%, 2.3-6.5%, 2.4-6.5%, 2.5-6.5%, 2.6-6.5%, 2.7-6.5%, 2.8-6.5%, 2.9-6.5%, 3-6,5%, 3.1-6.5%, 3.2-6.5%, 3.3-6.5%, 3.4-6.5%, 3,5-6,5%, 3.6-6.5%, 3.7-6.5%, 3.8-6.5%, 3.9-6.5%, 4-6.5%, 4.1-6.5%, 4.2-6.5%, 4.3-6.5%, 4.4-6.5%, 4.5-6.5%, 4.6-6.5%, 4.7-6.5%, 4.8-6.5%, 4.9-6.5%, 5-6.5%, 5.1-6.5%, 5.2-6.5%, 5.3-6.5%, 5.4-6.5%, 5.5-6.5%, 5.6-6.5%, 5.7-6.5%, 5.8-6.5%, 5.9-6.5%, 6-6.5%, 6.1-6.5%, 6.2-6.5%, 6.3-6.5%, 6.4-6.5%, 0-7%. 0.1-7%, 0.2-7%, 0.3-7%, 0.4-7%, 0.5-7%, 0.6-7%, 0.7-7%, 0,8-7%, 0.9-7%, 1-7%, 1.1-7%, 1.2-7%, 1.3-7%, 1.4-7%, 1.5-7%, 1.6-7%, 1.7-7%, 1.8-7%, 1.9-7%, 2-7%, 2.1-7%, 2.2-7%, 2.3-7%, 2.4-7%, 2.5-7%, 2.6-7%, 2.7-7%, 2.8-7%, 2.9-7%, 3-7%, 3.1-7%, 3.2-7%, 3.3-7%, 3.4-7%, 3.5-7%, 3.6-7%, 3.7-7%, 3.8-7%, 3.9-7%, 4-7%, 4.1-7%, 4.2-7%, 4.3-7%, 4.4-7%, 4.5-7%, 4.6-7%, 4.7-7%, 4.8-7%, 4.9-7%, 5-7%, 5.1-7%, 5.2-7%, 5.3-7%, 5.4-7%, 5.5-7%, 5.6-7%, 5.7-7%, 5.8-7%, 5.9-7%, 6-7%, 6.1-7%, 6.2-7%, 6.3-7%, 6.4-7%, 6.5-7%, 6.6-7%, 6.7-7%, 6.8-7%, 6.9-7%, 0-7.5%, 0.1-7.5%, 0.2-7.5%, 0.3-7.5%, 0.4-7.5%, 0.5-7.5%, 0.6-7.5%, 0.7-7.5%, 0.8-7.5%, 0.9-7.5%, 1-7.5%, 1.1-7.5%, 1.2-7.5%, 1.3-7.5%, 1.4-7.5%, 1.5-7.5%, 1.6-7.5%, 1.7-7.5%, 1.8-7.5%, 1.9-7.5%, 2-7.5%, 2.1-7.5%,2.2-7.5%, 2.3-7.5%, 2.4-7.5%, 2.5-7.5%, 2.6-7.5%, 2.7-7.5%, 2.8-7.5%, 2.9-7.5%, 3-7.5%, 3.1-7.5%, 3.2-7.5%, 3.3-7.5%, 3.4-7.5%, 3.5-7.5%, 3.6-7.5%, 3.7-7.5%, 3.8-7.5%, 3.9-7.5%, 4-7.5%, 4.1-7.5%, 4.2-7.5%, 4.3-7.5%, 4.4-7.5%, 4.5-7.5%, 4.6-7.5%, 4.7-7.5%, 4.8-7.5%, 4.9-7.5%, 5-7.5%, 5.1-7.5%, 5.2-7.5%, 5.3-7.5%, 5.4-7.5%, 5.5-7.5%, 5.6-7.5%, 5.7-7.5%, 5.8-7.5%, 5.9-7.5%, 6-7.5%, 6.1-7.5%, 6.2-7.5%, 6.3-7.5%, 6.4-7.5%, 6.5-7.5%, 6.6-7.5%, 6.7-7.5%, 6.8-7.5%, 6.9-7.5%, 7-7.5%, 7.1-7.5%, 7.2-7.5%, 7.3-7.5%, 7.4-7.5%, 0-8%, 0.1-8%, 0.2-8%, 0.3-8%, 0.4-8%, 0.5-8%, 0.6-8%, 0.7-8%, 0.8-8%, 0.9-8%, 1-8%, 1.1-8%, 1.2-8%, 1.3-8%, 1.4-8%, 1.5-8%, 1.6-8%, 1.7-8%, 1.8-8%, 1.9-8%, 2-8%, 2.1-8%, 2.2-8%, 2.3-8%, 2.4-8%, 2.5-8%, 2.6-8%, 2.7-8%, 2.8-8%, 2.9-8%, 3-8%, 3.1-8%, 3.2-8%, 3.3-8%, 3.4-8%, 3.5-8%, 3.6-8%, 3.7-8%, 3.8-8%, 3.9-8%, 4-8%, 4.1-8%, 4.2-8%, 4.3-8%, 4.4-8%, 4.5-8%, 4.6-8%, 4.7-8%, 4.8-8%, 4.9-8%, 5-8%, 5.1-8%, 5.2-8%, 5.3-8%, 5.4-8%, 5.5-8%, 5.6-8%, 5.7-8%, 5.8-8%, 5.9-8%, 6-8%, 6.1-8%, 6.2-8%, 6.3-8%, 6.4-8%, 6.5-8%, 6.6-8%, 6.7-8%, 6.8-8%, 6.9-8%, 7-8%, 7.1-8%, 7.2-8%, 7.3-8%, 7.4-8%, 7.5-8%, 7.6-8%, 7.7-8%, 7.8-8%, 7.9-8%, 0-8.5%, 0.1-8.5%, 0.2-8.5%. 0.3-8.5%, 0.4-8.5%, 0.5-8.5%, 0.6-8.5%, 0.7-8.5%, 0.8-8.5%, 0.9-8.5%, 1-8.5%, 1.1-8.5%, 1.2-8.5%, 1.3-8.5%, 1.4-8.5%, 1.5-8.5%, 1.6-8.5%, 1.7-8.5%, 1.8-8.5%, 1.9-8.5%, 2-8.5%, 2.1-8.5%, 2.2-8.5%, 2.3-8.5%, 2.4-8.5%, 2.5-8.5%, 2.6-8.5%, 2.7-8.5%, 2.8-8.5%, 2.9-8.5%, 3-8.5%, 3.1-8.5%, 3.2-8.5%, 3.3-8.5%, 3.4-8.5%, 3.5-8.5%, 3.6-8.5%, 3.7-8.5%, 3.8-8.5%, 3.9-8.5%, 4-8.5%, 4.1-8.5%, 4.2-8.5%, 4.3-8.5%, 4.4-8.5%, 4.5-8.5%, 4.6-8.5%, 4.7-8.5%, 4.8-8.5%, 4.9-8.5%, 5-8.5%. 5.1-8.5%, 5.2-8.5%, 5.3-8.5%, 5.4-8.5%, 5.5-8.5%, 5.6-8.5%, 5.7-8.5%, 5.8-8.5%, 5.9-8.5%, 6-8.5%, 6.1-8.5%, 6.2-8.5%, 6.3-8.5%, 6.4-8.5%, 6.5-8.5%, 6.6-8.5%, 6.7-8.5%, 6.8-8.5%, 6.9-8.5%, 7-8.5%, 7.1-8.5%, 7.2-8.5%, 7.3-8.5%, 7.4-8.5%, 7.5-8.5%, 7.6-8.5%, 7.7-8.5%, 7.8-8.5%, 7.9-8.5%, 8-8.5%, 8.1-8.5%, 8.2-8.5%, 8.3-8.5%, 8.4-8.5%, 0-9%, 0.1-9%, 0.2-9%, 0.3-9%, 0.4-9%, 0.5-9%, 0.6-9%, 0.7-9%, 0.8-9%, 0.9-9%, 1-9%, 1.1-9%, 1.2-9%, 1.3-9%, 1.4-9%, 1.5-9%, 1.6-9%, 1.7-9%, 1.8-9%, 1.9-9%, 2-9%, 2.1-9%, 2.2-9%, 2.3-9%, 2.4-9%, 2.5-9%, 2.6-9%, 2.7-9%, 2.8-9%, 2.9-9%, 3-9%, 3.1-9%, 3.2-9%, 3.3-9%, 3.4-9%, 3.5-9%, 3.6-9%, 3.7-9%, 3.8-9%, 3.9-9%, 4-9%, 4.1-9%, 4.2-9%, 4.3-9%, 4.4-9%, 4.5-9%, 4.6-9%, 4.7-9%, 4.8-9%, 4.9-9%, 5-9%, 5.1-9%, 5.2-9%, 5.3-9%, 5.4-9%, 5.5-9%, 5.6-9%, 5.7-9%, 5.8-9%, 5.9-9%, 6-9%, 6.1-9%, 6.2-9%, 6.3-9%, 6.4-9%, 6.5-9%, 6.6-9%, 6.7-9%, 6.8-9%, 6.9-9%, 7-9%, 7.1-9%, 7.2-9%, 7.3-9%, 7.4-9%, 7.5-9%, 7.6-9%, 7.7-9%, 7.8-9%, 7.9-9%, 8-9%, 8.1-9%, 8.2-9%, 8.3-9%, 8.4-9%, 8.5-9%, 8.6-9%, 8.7-9%, 8.8-9%, 8.9-9%, 0-9.5%, 0.1-9.5%, 0.2-9.5%, 0,3-9,5%, 0.4-9.5%, 0.5-9.5%, 0.6-9.5%, 0.7-9.5%, 0.8-9.5%, 0.9-9.5%, 1-9.5%, 1.1-9.5%, 1.2-9.5%, 1.3-9.5%, 1.4-9.5%, 1.5-9.5%, 1.6-9.5%, 1.7-9.5%, 1.8-9.5%, 1.9-9.5%, 2-9.5%, 2.1-9.5%, 2.2-9.5%, 2.3-9.5%, 2.4-9.5%, 2.5-9.5%, 2.6-9.5%, 2.7-9.5%, 2.8-9.5%, 2.9-9.5%, 3-9.5%, 3.1-9.5%, 3.2-9.5%, 3.3-9.5%, 3.4-9.5%, 3.5-9.5%, 3.6-9.5%, 3.7-9.5%, 3.8-9.5%, 3.9-9.5%, 4-9.5%, 4.1-9.5%, 4.2-9.5%, 4.3-9.5%, 4.4-9.5%, 4.5-9.5%, 4.6-9.5%, 4.7-9.5%, 4.8-9.5%, 4.9-9.5%, 5-9.5%, 5.1-9.5%, 5.2-9.5%, 5.3-9.5%, 5.4-9.5%, 5.5-9.5%, 5.6-9.5%, 5.7-9.5%, 5.8-9.5%, 5.9-9.5%, 6-9.5%, 6.1-9.5%, 6.2-9.5%, 6.3-9.5%, 6.4-9.5%, 6.5-9.5%, 6.6-9.5%, 6.7-9.5%, 6.8-9.5%, 6.9-9.5%, 7-9.5%, 7.1-9.5%, 7.2-9.5%, 7.3-9.5%, 7.4-9.5%, 7.5-9.5%, 7.6-9.5%, 7.7-9.5%, 7.8-9.5%, 7.9-9.5%, 8-9.5%, 8.1-9.5%, 8.2-9.5%, 8.3-9.5%, 8.4-9.5%, 8,5-9.5%, 8.6-9.5%, 8.7-9.5%, 8.8-9.5%, 8.9-9.5%, 9-9.5%, 9.1-9.5%, 9.2-9.5%, 9.3-9.5%, 9.4-9.5%, 0-10%, 0.1-10%, 0.2-10%, 0.340%, 0.4-10%, 0.5-10%, 0.6-10%, 0.7-10%, 0.8-10%, 0.9-10%, 1-10%, 1.1-10%, 1.2-10%, 1.3-10%, 1.4-10%, 1.5-10%, 1.6-10%, 1.7-10%, 1.8-10%, 1.9-10%, 2-10%, 2.1-10%, 2.2-10%, 2.3-10%, 2.4-10%, 2.5-10%, 2.6-10%, 2.7-10%, 2.8-10%, 2.9-10%, 3-10%, 3.1-10%, 3.2-10%, 3.3-10%, 3.440%, 3.5-10%, 3.6-10%, 3.7-10%, 3.8-10%, 3.9-10%, 4-10%, 4.1-10%, 4.2-10%, 4.3-10%, 4.4-10%, 4.5-10%, 4.6-10%, 4.7-10%, 4.8-10%, 4.9-10%, 5-10%, 5.1-10%, 5.2-10%, 5.3-10%, 5.4-10%, 5.5-10%, 5.6-10%, 5.7-10%, 5.8-10%, 5.9-10%, 6-10%, 6.1-10%, 6.2-10%, 6.3-10%, 6.4-10%, 6.5-10%, 6.6-10%, 6.7-10%, 6.8-10%, 6.9-10%, 7-10%, 7.1-10%, 7.2-10%, 7.3-10%, 7.4-10%, 7.5-10%, 7.6-10%, 7.7-10%, 7.8-10%, 7.9-10%, 8-10%, 8.1-10%, 8.2-10%, 8.3-10%, 8.4-10%, 8.5-10%, 8.6-10%, 8.7-10%, 8.8-10%, 8.9-10%, 9-10%, 9.1-10%, 9.2-10%, 9.3-10%, 9.4-10%, 9.5-10%, 9.6-10%, 9.7-10%, 9.8-10%, or 9.940% w/v.

In certain embodiments, the formulation may comprise 0-10% w/v of sucrose.

In certain embodiments, the formulation may comprise 0-9% w/v of sucrose.

In certain embodiments, the formulation may comprise 0-8% w/v of sucrose.

In certain embodiments, the formulation may comprise 0-7% w/v of sucrose.

In certain embodiments, the formulation may comprise 0-6% w/v of sucrose.

In certain embodiments, the formulation may comprise 0-5% w/v of sucrose.

In certain embodiments, the formulation may comprise 0-4% w/v of sucrose.

In certain embodiments, the formulation may comprise 0-3% w/v of sucrose.

In certain embodiments, the formulation may comprise 0-2% w/v of sucrose.

In certain embodiments, the formulation may comprise 0-1% w/v of sucrose.

In certain embodiments, the formulation may comprise 1% w/v of sucrose.

In certain embodiments, the formulation may comprise 2% w/v of sucrose.

In certain embodiments, the formulation may comprise 3% w/v of sucrose.

In certain embodiments, the formulation may comprise 4% w/v of sucrose.

In certain embodiments, the formulation may comprise 5% w/v of sucrose.

In certain embodiments, the formulation may comprise 6% w/v of sucrose.

In certain embodiments, the formulation may comprise 7% w/v of sucrose.

In certain embodiments, the formulation may comprise 8% w/v of sucrose.

In certain embodiments, the formulation may comprise 9% w/v of sucrose.

In certain embodiments, the formulation may comprise 10% w/v of sucrose.

### Trehalose

In certain embodiments, the formulation may comprise at least one sugar which is disaccharide such as, but not limited to, trehalose.

In certain embodiments, the formulation may comprise trehalose at 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, or 10% w/v.

In certain embodiments, the formulation may comprise trehalose in a range of 0-1%, 0.1-1%, 0.2-1%, 0.3-1%, 0.4-1%, 0.5-1%, 0.6-1%, 0.7-1%, 0.8-1%, 0.9-1%, 0-1.5%, 0.1-1.5%, 0.2-1.5%, 0.3-1.5%, 0.4-1.5%, 0.5-1.5%, 0.6-1.5%, 0.7-1.5%, 0.8-1.5%, 0.9-1.5%, 1-1.5%, 1.1-1.5%, 1.2-1.5%, 1.3-1.5%, 1.4-1.5%, 0-2%, 0.1-2%, 0.2-2%, 0.3-2%, 0.4-2%, 0.5-2%, 0.6-2%, 0.7-2%, 0.8-2%, 0.9-2%, 1-2%, 1,1-2%, 1.2-2%, 1.3-2%, 1.4-2%, 1.5-2%, 1.6-2%, 1.7-2%, 1.8-2%, 1.9-2%, 0-2.5%, 0.1-2.5%, 0.2-2.5%, 0.3-2.5%, 0.4-2.5%, 0.5-2.5%, 0.6-2.5%, 0.7-2.5%, 0.8-2.5%, 0.9-2.5%, 1-2.5%, 1.1-2.5%, 1.2-2.5%, 1.3-2.5%, 1.4-2.5%, 1.5-2.5%, 1.6-2.5%, 1.7-2.5%, 1.8-2.5%, 1.9-2.5%, 2-2.5%, 2.1-2.5%, 2.2-2.5%, 2.3-2.5%, 2.4-2.5%, 0-3%, 0.1-3%, 0.2-3%, 0.3-3%, 0.4-3%, 0.5-3%, 0.6-3%, 0.7-3%, 0.8-3%, 0.9-3%, 1-3%, 1.1-3%, 1.2-3%, 1.3-3%, 1.4-3%, 1.5-3%, 1.6-3%, 1.7-3%, 1.8-3%, 1.9-3%, 2-3%, 2.1-3%, 2.2-3%, 2.3-3%, 2.4-3%, 2.5-3%, 2.6-3%, 2.7-3%, 2.8-3%, 2.9-3%, 0-3.5%, 0.1-3.5%, 0.2-35%, 0.3-3.5%, 0.4-3.5%, 0.5-3.5%, 0.6-3.5%, 0.7-3.5%, 0.8-3.5%, 0.9-3.5%, 1-3.5%, 1.1-3.5%, 1.2-3.5%, 1.3-3.5%, 1.4-3.5%, 1.5-3.5%, 1.6-3.5%, 1.7-3.5%, 1.8-3.5%, 1.9-3.5%, 2-3.5%, 2.1-3.5%, 2.2-3.5%, 2.3-3.5%, 2.4-3.5%, 2.5-3.5%, 2.6-3.5%, 2.7-3.5%, 2.8-3.5%, 2.9-3.5%, 3-3.5%, 3.1-3.5%, 3.2-3.5%, 3.3-3.5%, 3.4-3.5%, 0-4%, 0.1-4%, 0.2-4%, 0.3-4%, 0.4-4%, 0.5-4%, 0.6-4%, 0.7-4%, 0.8-4%, 0.9-4%, 1-4%, 1.1-4%, 1.2-4%, 1.3-4%, 1.4-4%, 1.5-4%, 1.6-4%, 1.7-4%, 1.8-4%, 1.9-4%, 2-4%, 2.1-4%, 2.2-4%, 2.3-4%, 2.4-4%, 2.5-4%, 2.6-4%, 2.7-4%, 2.8-4%, 2.9-4%, 3-4%, 3.1-4%, 3.2-4%, 3.3-4%, 3.4-4%, 3.5-4%, 3.6-4%, 3.7-4%, 3.8-4%, 3.9-4%, 0-4.5%, 0.1-4.5%, 0.2-4.5%, 0.3-4.5%, 0.4-4.5%, 0.5-4.5%, 0.6-4.5%, 0.7-4.5%, 0.8-4.5%, 0.9-4.5%, 1-4.5%, 1.1-4.5%, 1.2-4.5%, 1.3-4.5%, 1.4-4.5%, 1.5-4.5%, 1.6-4.5%, 1.7-4.5%, 1.8-4.5%, 1.9-4.5%, 2-4.5%, 2.1-4.5%, 2.2-4.5%, 2.3-4.5%, 2.4-4.5%, 2.5-4.5%, 2.6-4.5%, 2.7-4.5%, 2.8-4.5%, 2.9-4.5%, 3-4.5%, 3.1-4.5%, 3.2-4.5%, 3.3-4.5%, 3.4-4.5%, 3.5-4.5%, 3.6-4.5%, 3.7-4.5%, 3.8-4.5%, 3.9-4.5%, 4-4.5%, 4.1-4.5%, 4.2-4.5%, 4.3-4.5%, 4.4-4.5%, 0-5%, 0.1-5%, 0.2-5%, 0.3-5%, 0.4-5%, 0.5-5%, 0.6-5%, 0.7-5%, 0.8-5%, 0.9-5%, 1-5%, 1.1-5%, 1.2-5%, 1.3-5%, 1.4-5%, 1.5-5%, 1.6-5%, 1.7-5%, 1.8-5%, 1.9-5%, 2-5%, 2.1-5%, 2.2-5%, 2.3-5%, 2.4-5%, 2.5-5%, 2.6-5%, 2.7-5%, 2.8-5%, 2.9-5%, 3-5%, 3.1-5%, 3.2-5%, 3.3-5%, 3.4-5%, 3.5-5%, 3.6-5%, 3.7-5%, 3.8-5%, 3.9-5%, 4-5%, 4.1-5%, 4.2-5%, 4.3-5%, 4.4-5%, 4.5-5%, 4.6-5%, 4.7-5%, 4.8-5%, 4.9-5%, 0-5.5%, 0.1-5.5%, 0.2-5.5%, 0.3-5.5%, 0.4-5.5%, 0.5-5.5%, 0.6-5.5%, 0.7-5.5%, 0.8-5.5%, 0.9-5.5%, 1-5.5%, 1.1-5.5%, 1.2-5.5%, 1.3-5.5%, 1.4-5.5%, 1.5-5.5%, 1.6-5.5%, 1.7-5.5%, 1.8-5.5%, 1.9-5.5%, 2-5.5%, 2.1-5.5%, 2.2-5.5%, 2.3-5.5%, 2.4-5.5%, 2.5-5.5%, 2.6-5.5%, 2.7-5.5%, 2.8-5.5%, 2.9-5.5%, 3-5.5%, 3.1-5.5%, 3.2-5.5%, 3.3-5.5%, 3.4-5.5%, 3.5-5.5%, 3.6-5.5%, 3.7-5.5%, 3.8-5.5%, 3.9-5.5%, 4-5.5%, 4.1-5.5%, 4.2-5.5%, 4.3-5.5%, 4.4-5.5%, 4.5-5.5%, 4.6-5.5%, 4.7-5.5%, 4.8-5.5%, 4.9-5.5%, 5-5.5%, 5.1-5.5%, 5.2-5.5%, 5.3-5.5%, 5.4-5.5%, 0-6%, 0.1-6%, 0.2-6%, 0.3-6%, 0.4-6%, 0.5-6%, 0.6-6%, 0.7-6%, 0.8-6%, 0.9-6%, 1-6%, 1.1-6%, 1.2-6%, 1.3-6%, 1.4-6%, 1.5-6%, 1.6-6%, 1.7-6%, 1.8-6%, 1.9-6%, 2-6%, 2.1-6%, 2.2-6%, 2.3-6%, 2.4-6%, 2.5-6%, 2.6-6%, 2.7-6%, 2.8-6%, 2.9-6%, 3-6%, 3.1-6%, 3.2-6%, 3.3-6%, 3.4-6%, 3.5-6%, 3.6-6%, 3.7-6%, 3.8-6%, 3.9-6%, 4-6%, 4.1-6%, 4.2-6%, 4.3-6%, 4.4-6%, 4.5-6%, 4.6-6%, 4.7-6%, 4.8-6%, 4.9-6%, 5-6%, 5.1-6%, 5.2-6%, 5.3-6%, 5.4-6%, 5.5-6%, 5.6-6%, 5.7-6%, 5.8-6%, 5.9-6%, 0-6,5%, 0.1-6.5%, 0.2-6.5%, 0.3-6.5%, 0.4-6.5% 0.6-6.5%, 0.7-6.5%, 0.8-6.5%, 0.9-6.5%, 1-6.5%, 1.1-6.5%, 1.2-6.5%, 1.3-6.5%, 1.4-6.5%, 1.5-6.5%, 1.6-6.5%, 1.7-6.5%, 1.8-6.5%, 1.9-6.5%, 2-6.5%, 2.1-6.5%, 2.2-6.5%, 2.3-6.5%, 2.4-6.5%, 2.5-6.5%, 2.6-6.5%, 2.7-6.5%, 2.8-6.5%, 2.9-6.5%, 3-6.5%, 3.1-6.5%, 3.2-6.5%, 3.3-6.5%, 3.4-6.5%. 3.5-6.5%, 3.6-6.5%, 3.7-6.5%, 3.8-6.5%, 3.9-6.5%, 4-6.5%, 4.1-6.5%, 4.2-6.5%, 4.3-6.5%, 4.4-6.5%, 4.5-6.5%, 4.6-6.5%, 4.7-6.5%, 4.8-6.5%, 4.9-6.5%, 5-6.5%, 5.1-6.5%, 5.2-6.5%, 5.3-6.5%, 5.4-6.5%, 5.5-6.5%, 5.6-6.5%, 5.7-6.5%, 5.8-6.5%, 5.9-6.5%, 6-6.5%, 6.1-6.5%, 6.2-6.5%, 6.3-6.5%, 6.4-6.5%, 0-7%, 0.1-7%, 0.2-7%, 0.3-7%, 0.4-7%, 0.5-7%, 0.6-7%, 0.7-7%, 0.8-7%, 0.9-7%, 1-7%, 1.1-7%, 1.2-7%, 1.3-7%, 1.4-7%, 1.5-7%, 1.6-7%, 1.7-7%, 1.8-7%, 1.9-7%, 2-7%, 2.1-7%, 2.2-7%, 2.3-7%, 2.4-7%, 2.5-7%, 2.6-7%, 2.7-7%, 2.8-7%, 2.9-7%, 3-7%, 3.1-7%, 3.2-7%, 3.3-7%, 3.4-7%, 3.5-7%, 3.6-7%, 3.7-7%, 3.8-7%, 3.9-7%, 4-7%, 4.1-7%, 4.2-7%, 4.3-7%, 4.4-7%, 4.5-7%, 4.6-7%, 4.7-7%, 4.8-7%, 4.9-7%, 5-7%, 5.1-7%, 5.2-7%, 5.3-7%, 5.4-7%, 5.5-7%, 5.6-7%, 5.7-7%, 5.8-7%, 5.9-7%, 6-7%, 6.1-7%, 6.2-7%, 6.3-7%, 6.4-7%, 6.5-7%, 6.6-7%, 6.7-7%, 6.8-7%, 6.9-7%, 0-7.5%, 0.1-7.5%, 0.2-7.5%, 0.3-7.5%, 0.4-7.5%, 0.5-7.5%. 0.6-7.5%, 0.7-7.5%, 0.8-7.5%, 0.9-7.5%, 1-7.5%, 1.1-7.5%, 1.2-7.5%, 1.3-7.5%, 1.4-7.5%, 1.5-7.5%, 1.6-7.5%, 1.7-7.5%, 1.8-7.5%, 1.9-7.5%, 2-7.5%, 2.1-7.5%, 2.2-7.5%, 2.3-7.5%, 2.4-7.5%, 2.5-7.5%, 2.6-7.5%, 2.7-7.5%, 2.8-7.5%, 2.9-7.5%, 3-7.5%, 3.1-7.5%, 3.2-7.5%, 3.3-7.5%, 3.4-7.5%, 3.5-7.5%, 3.6-7.5%, 3.7-7.5%, 3.8-7.5%, 3.9-7.5%, 4-7.5%, 4.1-7.5%, 4.2-7.5%, 4.3-7.5%, 4.4-7.5%, 4.5-7.5%, 4.6-7.5%, 4.7-7.5%, 4.8-7.5%, 4.9-7.5%, 5-7.5%, 5.1-7.5%, 5.2-7.5%, 5.3-7.5%, 5.4-7.5%, 5.5-7.5%, 5.6-7.5%, 5.7-7.5%, 5.8-7.5%, 5.9-7.5%, 6-7.5%, 6.1-7.5%, 6.2-7.5%, 6.3-7.5%, 6.4-7.5%, 6.5-7.5%, 6.6-7.5%, 6.7-7.5%, 6.8-7.5%, 6.9-7.5%, 7-7.5%, 7.1-7.5%, 7.2-7.5%, 7.3-7.5%, 7.4-7.5%, 0-8%, 0.1-8%, 0.2-8%, 0.3-8%, 0.4-8%, 0.5-8%, 0.6-8%, 0.7-8%, 0.8-8%, 0.9-8%, 1-8%, 1.1-8%, 1.2-8%, 1,3-8%, 1.4-8%, 1.5-8%, 1.6-8%, 1.7-8%, 1.8-8%, 1.9-8%, 2-8%, 2.1-8%, 2.2-8%, 2.3-8%, 2.4-8%, 2.5-8%, 2.6-8%, 2.7-8%, 2.8-8%, 2.9-8%, 3-8%, 3.1-8%, 3.2-8%, 3.3-8%, 3.4-8%, 3.5-8%, 3.6-8%, 3.7-8%, 3.8-8%, 3.9-8%, 4-8%, 4.1-8%, 4.2-8%, 4.3-8%, 4.4-8%, 4.5-8%, 4.6-8%, 4.7-8%, 4.8-8%, 4.9-8%, 5-8%, 5.1-8%, 5.2-8%, 5.3-8%, 5.4-8%, 5.5-8%, 5.6-8%, 5.7-8%, 5.8-8%, 5.9-8%, 6-8%, 6.1-8%, 6.2-8%, 6.3-8%, 6.4-8%, 6.5-8%, 6.6-8%, 6.7-8%, 6.8-8%, 6.9-8%, 7-8%, 7.1-8%, 7.2-8%, 7.3-8%, 7.4-8%, 7.5-8%, 7.6-8%, 7.7-8%, 7.8-8%, 7.9-8%, 0-8.5%, 0.1-8.5%, 0.3-8.5%, 0.4-8.5%, 0.5-8.5%, 0.6-8.5%, 0.7-8.5%, 0.8-8.5%, 0.9-8.5%, 1-8.5%, 1.1-8.5%, 1.2-8.5%, 1.3-8.5%, 1.4-8.5%, 1.5-8.5%, 1.6-8.5%, 1.7-8.5%, 1.8-8.5%, 1.9-8.5%, 2-8.5%, 2.1-8.5%, 2.2-8.5%, 2.3-8.5%.2.4-8.5%. 2.5-8.5%, 2.6-8.5%, 2.7-8.5%, 2.8-8.5%, 2.9-8.5%, 3-8.5%, 3.1-8.5%, 3.2-8.5%, 3.3-8.5%, 3.4-8.5%, 3.5-8.5%, 3.6-8.5%, 3.7-8.5%, 3.8-8.5%, 3.9-8.5%, 4-8.5%, 4.1-8.5%, 4.2-8.5%, 4.3-8.5%, 4.4-8.5%, 4.5-8.5%, 4.6-8.5%, 4.7-8.5%, 4.8-8.5%, 4.9-8.5%, 5-8.5%, 5.1-8.5%, 5.2-8.5%, 5.3-8.5%, 5.4-8.5%, 5.5-8.5%, 5.6-8.5%, 5.7-8.5%, 5.8-8.5%, 5.9-8.5%, 6-8.5%, 6.1-8.5%, 6.2-8.5%, 6.3-8.5%, 6.4-8.5%, 6.5-8.5%, 6.6-8.5%, 6.7-8.5%, 6.8-8.5%, 6.9-8.5%, 7-8.5%, 7.1-8.5%, 7.2-8.5%, 7.3-8.5%, 7.4-8.5%, 7.5-8.5%, 7.6-8.5%, 7.7-8.5%, 7.8-8.5%, 7.9-8.5%, 8-8.5%, 8.1-8.5%, 8.2-8.5%, 8.3-8.5%, 8.4-8.5%, 0-9%, 0.1-9%, 0.2-9%, 0.3-9%, 0.4-9%, 0.5-9%, 0.6-9%, 0.7-9%, 0.8-9%, 0.9-9%, 1-9%, 1.1-9%, 1.2-9%, 1.3-9%, 1.4-9%, 1.5-9%, 1.6-9%, 1.7-9%, 1.8-9%, 1.9-9%, 2-9%, 2.1-9%, 2.2-9%, 2.3-9%, 2.4-9%, 2.5-9%, 2.6-9%, 2.7-9%, 2.8-9%, 2.9-9%, 3-9%, 3.1-9%, 3.2-9%, 3.3-9%, 3.4-9%, 3.5-9%, 3.6-9%, 3.7-9%, 3.8-9%, 3.9-9%, 4-9%, 4.1-9%, 4.2-9%, 4.3-9%, 4.4-9%.4.5-9%, 4.6-9%, 4.7-9%, 4.8-9%, 4.9-9%, 5-9%, 5.1-9%, 5.2-9%, 5.3-9%, 5.4-9%, 5.5-9%, 5.6-9%, 5.7-9%, 5.8-9%, 5.9-9%, 6-9%, 6.1-9%, 6.2-9%, 6.3-9%, 6.4-9%, 6.5-9%, 6.6-9%, 6.7-9%, 6.8-9%, 6.9-9%, 7-9%, 7.1-9%, 7.2-9%, 7.3-9%, 7.4-9%, 7.5-9%, 7.6-9%, 7.7-9%, 7.8-9%, 7.9-9%, 8-9%, 8.1-9%, 8.2-9%, 8.3-9%, 8.4-9%, 8.5-9%, 8.6-9%, 8.7-9%, 8.8-9%, 8.9-9%, 0-9.5%, 0.1-9.5%, 0.2-9.5%, 0.3-9.5%, 0.4-9.5%, 0.5-9.5%, 0.6-9.5%, 0.7-9.5%, 0.8-9.5%, 0.9-9.5%, 1-9.5%, 1.1-9.5%, 1.2-9.5%, 1.3-9.5%, 1.4-9,5%, 1.5 -9.5%, 1.6-9.5%, 1.7-9.5%, 1.8-9.5%, 1.9-9.5%, 2-9.5%, 2.1-9.5%, 2.2-9.5%, 2.3-9.5%, 2.4-9.5%, 2.5-9.5%, 2.6-9.5%, 2.7-9.5%, 2.8-9.5%, 2.9-9.5%, 3-9.5%, 3.1-9.5%, 3.2-9.5%, 3.3-9.5%, 3.4-9.5%, 3.5-9.5%, 3.6-9.5%, 3.7-9.5%, 3.8-9.5%, 3.9-9.5%, 4-9.5%, 4.1-9.5%, 4.2-9.5%, 4.3-9.5%, 4.4-9.5%, 4.5-9.5%, 4.6-9.5%, 4.7-9.5%, 4,8-9,5%, 4,9-9,5%, 5-9.5%, 5.1-9.5%, 5.2-9.5%, 5.3-9.5%, 5.4-9.5%, 5.5-9.5%, 5.6-9.5%, 5.7-9.5%, 5.8-9.5%, 5.9-9.5%, 6-9.5%, 6.1-9.5%, 6.2-9.5%, 6.3-9.5%, 6.4-9.5%, 6.5-9.5%, 6.6-9.5%, 6.7-9.5%, 6.8-9.5%, 6.9-9.5%, 7-9.5%, 7.1-9.5%, 7.2-9.5%, 7.3-9.5%, 7.4-9.5%, 7.5-9.5%, 7.6-9.5%, 7.8-9.5%, 7.9-9.5%, 8-9.5%, 8.1-9.5%, 8.2-9.5%, 8.3-9.5%, 8.4-9.5%, 8,5-9.5%, 8.6-9.5%, 8.7-9.5%, 8.8-9.5%, 8.9-9.5%, 9-9.5%, 9.1-9.5%, 9.2-9.5%, 9.3-9.5%, 9.4-9.5%, 0-10%, 0.1-10%, 0.2-10%, 0.3-10%, 0.4-10%, 0.5-10%, 0.6-10%, 0.7-10%, 0.8-10%, 0.9-10%, 1-10%, 1.1-10%, 1.2-10%, 1.3-10%, 1.4-10%, 1.5-10%, 1.6-10%, 1.7-10%, 1.8-10%, 1.9-10%, 2-10%, 2.1-10%, 2.2-10%, 2.3-10%, 2.4-10%, 2.5-10%, 2.6-10%, 2.7-10%, 2.8-10%, 2.9-10%, 3-10%, 3.1-10%, 3.2-10%, 3.3-10%, 3.4-10%, 3.5-10%, 3.6-10%, 3.7-10%, 3.8-10%, 3.9-10%, 4-10%, 4.1-10%, 4.2-10%, 4.3-10%, 4.4-10%, 4.5-10%, 4.6-10%, 4.7-10%, 4.8-10%, 4.9-10%, 5-10%, 5.1-10%, 5.2-10%, 5.3-10%, 5.4-10%, 5.5-10%, 5.6-10%, 5.7-10%, 5.8-10%, 5.9-10%, 6-10%, 6.1-10%, 6.2-10%, 6.3-10%, 6.4-10%, 6.5-10%, 6.6-10%, 6.7-10%, 6.8-10%, 6.9-10%, 7-10%, 7.1-10%, 7.2-10%, 7.3-10%, 7.4-10%, 7.5-10%, 7.6-10%, 7.7-10%, 7.8-10%, 7.9-10%, 8-10%, 8.1-10%, 8.2-10%, 8.3-10%, 8.4-10%, 8.5-10%, 8.6-10%, 8.7-10%, 8.8-10%, 8.9-10%, 9-10%, 9.1-10%, 9.2-10%, 9.3-10%, 9.4-10%, 9.5-10%, 9.6-10%, 9.7-10%, 9.8-10%, or 9.9-10% w/v.

In certain embodiments, the formulation may comprise 0-10% w/v of trehalose.

In certain embodiments, the formulation may comprise 0-9% w/v of trehalose.

In certain embodiments, the formulation may comprise 0-8% w/v of trehalose.

In certain embodiments, the formulation may comprise 0-7% w/v of trehalose.

In certain embodiments, the formulation may comprise 0-6% w/v of trehalose.

In certain embodiments, the formulation may comprise 0-5% w/v of trehalose.

In certain embodiments, the formulation may comprise 0-4% w/v of trehalose.

In certain embodiments, the formulation may comprise 0-3% w/v of trehalose.

In certain embodiments, the formulation may comprise 0-2% w/v of trehalose.

In certain embodiments, the formulation may comprise 0-1% w/v of trehalose.

In certain embodiments, the formulation may comprise 1% w/v of trehalose.

In certain embodiments, the formulation may comprise 2% w/v of trehalose.

In certain embodiments, the formulation may comprise 3% w/v of trehalose.

In certain embodiments, the formulation may comprise 4% w/v of trehalose.

In certain embodiments, the formulation may comprise 5% w/v of trehalose.

In certain embodiments, the formulation may comprise 6% w/v of trehalose.

In certain embodiments, the formulation may comprise 7% wily of trehalose.

In certain embodiments, the formulation may comprise 8% w/v of trehalose.

In certain embodiments, the formulation may comprise 9% w/v of trehalose.

In certain embodiments, the formulation may comprise 10% w/v of trehalose.

### Sorhitol

In certain embodiments, the formulation may comprise at least one sugar substitute (e.g., a sugar alcohol) which is sorbitol.

In certain embodiments, the formulation may comprise sorbitol at 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 6.1%, 6.2%, 6.3%, 6.4%, 6.5%, 6.6%, 6.7%, 6.8%, 6.9%, 7%, 7.1%, 7.2%, 7.3%, 7.4%, 7.5%, 7.6%, 7.7%, 7.8%, 7.9%, 8%, 8.1%, 8.2%, 8.3%, 8.4%, 8.5%, 8.6%, 8.7%, 8.8%, 8.9%, 9%, 9.1%, 9.2%, 9.3%, 9.4%, 9.5%, 9.6%, 9.7%, 9.8%, 9.9%, or 10% w/v.

In certain embodiments, the formulation may comprise sorbitol in a range of 0-1%, 0.1-1%, 0.2-1%, 0.3-1%, 0.4-1%, 0.5-1%, 0.6-1%, 0.7-1%, 0.8-1%, 0.9-1%, 0-1.5%, 0.1-1.5%, 0.2-1.5%, 0.3-1.5%, 0.4-1.5%, 0.5-1.5%, 0.6-1.5%, 0.7-1.5%, 0.8-1.5%, 0.9-1.5%, 1-1.5%, 1.1-1.5%, 1.2-1.5%, 1.3-1.5%, 1.4-1.5%, 0-2%, 0.1-2%, 0.2-2%, 0.3-2%, 0.4-2%, 0.5-2%, 0.6-2%, 0.7-2%, 0.8-2%, 0.9-2%, 1-2%, 1,1-2%, 1.2-2%, 1.3-2%, 1.4-2%, 1.5-2%, 1.6-2%, 1.7-2%, 1.8-2%, 1.9-2%, 0-2.5%, 0.1-2.5%, 0.2-2.5%, 0.3-2.5%, 0.4-2.5%, 0.5-2.5%, 0.6-2.5%, 0.7-2.5%, 0.8-2.5%, 0.9-2.5%, 1-2.5%, 1.1-2.5%, 1.2-2.5%, 1.3-2.5%, 1.4-2.5%, 1.5-2.5%, 1.6-2.5%, 1.7-2.5%, 1.8-2.5%, 1.9-2.5%, 2-2.5%, 2.1-2.5%, 2.2-2.5%, 2.3-2.5%, 2.4-2.5%, 0-3%, 0.1-3%, 0.2-3%, 0.3-3%, 0.4-3%, 0.5-3%, 0.6-3%, 0.7-3%, 0.8-3%, 0.9-3%, 1-3%, 1.1-3%, 1.2-3%, 1.3-3%, 1.4-3%, 1.5-3%, 1.6-3%, 1.7-3%, 1.8-3%, 1.9-3%, 2-3%, 2.1-3%, 2.2-3%, 2.3-3%, 2.4-3%, 2.5-3%, 2.6-3%, 2.7-3%, 2.8-3%, 2.9-3%, 0-3.5%, 0.1-3.5%, 0.2-3.5%, 0.3-3.5%, 0.4-3.5%, 0.5-3.5%, 0.6-3.5%,0.7-3.5%, 0.8-3.5%, 0.9-3.5%, 1-3.5%, 1.1-3.5%, 1.2-3.5%, 1.3-3.5%, 1.4-3.5%, 1.5-3.5%, 1.6-3.5%, 1.7-3.5%, 1.8-3.5%, 1.9-3.5%, 2-3.5%, 2.1-3.5%, 2.2-3.5%, 2.3-3.5%, 2.4-3.5%, 2.5-3.5%, 2.6-3.5%, 2.7-3.5%, 2.8-3.5%, 2.9-3.5%, 3-3.5%, 3.1-3.5%, 3.2-3.5%, 3.3-3.5%, 3.4-3.5%, 0-4%, 0.1-4%, 0.2-4%, 0.3-4%, 0.4-4%, 0.5-4%, 0.6-4%, 0.7-4%, 0.8-4%, 0.9-4%, 1-4%, 1.1-4%, 1.2-4%, 1.3-4%, 1.4-4%, 1.5-4%, 1.6-4%, 1.7-4%, 1.8-4%, 1.9-4%, 2-4%, 2.1-4%, 2.2-4%, 2.3-4%, 2.4-4%, 2.5-4%, 2.6-4%, 2.7-4%, 2.8-4%, 2.9-4%, 3-4%, 3.1-4%, 3.2-4%, 3.3-4%, 3.4-4%, 3.5-4%, 3.6-4%, 3.7-4%, 3.8-4%, 3.9-4%, 0-4.5%, 0.1-4.5%, 0.2-4.5%, 0.3-4.5%, 0.4-4.5%, 0.5-4.5%, 0.6-4.5%, 0.7-4.5%, 0.8-4.5%, 0.9-4.5%, 1-4.5%, 1.1-4.5%, 1.2-4.5%, 1.3-4.5%, 1.4-4.5%, 1.5-4.5%, 1.6-4.5%, 1.7-4.5%, 1.8-4.5%, 1.9-4.5%, 2-4.5%, 2.1-4.5%, 2.2-4.5%, 2.3-4.5%, 2.4-4.5%, 2.5-4.5%, 2.6-4.5%, 2.7-4.5%, 2.8-4.5%, 2.9-4.5%, 3-4.5%, 3.1-4.5%, 3.2-4.5%, 3.3-4.5%, 3.4-4.5%, 3.5-4.5%, 3.6-4.5%, 3.7-4.5%, 3.8-4.5%, 3.9-4.5%, 4-4.5%, 4.1-4.5%, 4.2-4.5%, 4.3-4.5%, 4.4-4.5%, 0-5%, 0.1-5%, 0.2-5%, 0.3-5%, 0.4-5%, 0.5-5%, 0.6-5%, 0.7-5%, 0.8-5%, 0.9-5%, 1-5%, 1.1-5%, 1.2-5%, 1.3-5%, 1.4-5%, 1.5-5%, 1.6-5%, 1.7-5%, 1.8-5%, 1.9-5%, 2-5%, 2.1-5%, 2.2-5%, 2.3-5%, 2.4-5%, 2.5-5%, 2.6-5%, 2.7-5%, 2.8-5%, 2.9-5%, 3-5%, 3.1-5%, 3.2-5%, 3.3-5%, 3.4-5%, 3.5-5%, 3.6-5%, 3.7-5%, 3.8-5%, 3.9-5%, 4-5%, 4.1-5%, 4.2-5%, 4.3-5%, 4.4-5%, 4.5-5%, 4.6-5%, 4.7-5%, 4.8-5%, 4.9-5%, 0-5.5%, 0.1-5.5%, 0.2-5.5%, 0.3-5.5%, 0.4-5.5%, 0.5-5.5%, 0.6-5.5%, 0.7-5.5%, 0.8-5.5%, 0.9-5.5%, 1-5.5%, 1.1-5.5%, 1.2-5.5%, 1.3-5.5%, 1.4-5.5%, 1.5-5.5%, 1.6-5.5%, 1.7-5.5%, 1.8-5.5%, 1.9-5.5%, 2-5.5%, 2.1-5.5%, 2.2-5.5%, 2.3-5.5%, 2.4-5.5%, 2.5-5.5%, 2.6-5.5%, 2.7-5.5%, 2.8-5.5%, 2.9-5.5%, 3-5.5%, 3.1-5.5%, 3.2-5.5%, 3.3-5.5%, 3.4-5.5%, 3.5-5.5%, 3.6-5.5%, 3.7-5.5%, 3.8-5.5%, 3.9-5.5%, 4-5.5%, 4.1-5.5%, 4.2-5.5%, 4.3-5.5%, 4.4-5.5%, 4.5-5.5%, 4.6-5.5%, 4.7-5.5%, 4.8-5.5%, 4.9-5.5%, 5-5.5%, 5.1-5.5%, 5.2-5.5%, 5.3-5.5%, 5.4-5.5%, 0-6%, 0.1-6%, 0.2-6%, 0.3-6%, 0.4-6%, 0.5-6%, 0.6-6%, 0.7-6%, 0.8-6%, 0.9-6%, 1-6%, 1.1-6%, 1.2-6%, 1.3-6%, 1.4-6%, 1.5-6%, 1.6-6%, 1.7-6%, 1.8-6%, 1.9-6%, 2-6%, 2.1-6%, 2.2-6%, 2.3-6%, 2.4-6%, 2.5-6%, 2.6-6%, 2.7-6%, 2.8-6%, 2.9-6%, 3-6%, 3.1-6%, 3.2-6%, 3.3-6%, 3.4-6%, 3.5-6%, 3.6-6%, 3.7-6%, 3.8-6%, 3.9-6%, 4-6%, 4.1-6%, 4.2-6%, 4.3-6%, 4.4-6%, 4.5-6%, 4.6-6%, 4.7-6%, 4.8-6%, 4.9-6%, 5-6%, 5.1-6%, 5.2-6%, 5.3-6%, 5.4-6%, 5.5-6%, 5.6-6%, 5.7-6%, 5.8-6%, 5.9-6%, 0-6.5%, 0.1-6.5%, 0.2-6.5%, 0.3-6.5%, 0.4-6.5%, 0.5-6.5%, 0.6-6.5%, 0.7-6.5%, 0.8-6.5%, 0.9-6.5%, 1-6.5%, 1.1-6.5%, 1.2-6.5%, 1.3-6.5%, 1.4-6.5%, 1.5-6.5%, 1.6-6.5%, 1.7-6.5%, 1.8-6.5%, 19-6.5%, 2-6.5%, 2.1-6.5%, 2.2-6.5%, 2.3-6.5%, 2.4-6.5%, 2.5-6.5%, 2.6-6.5%, 2.7-6.5%, 2.8-6.5%, 2.9-6.5%, 3-6.5%, 3.1-6.5%, 3.2-6.5%, 3.3-6.5%, 3.4-6.5%, 3.5-6.5%, 3.6-6.5%, 3.7-6.5%, 3.8-6.5%, 3.9-6.5%, 4-6.5%, 4.1-6.5%, 4.2-6.5%, 4.3-6.5%, 4.4-6.5%, 4.5-6.5%, 4.6-6.5%, 4.7-6.5%, 4.8-6.5%, 4.9-6.5%, 5-6.5%, 5.1-6.5%, 5.2-6.5%, 5.3-6.5%, 5.4-6.5%, 5.5-6.5%, 5.6-6.5%, 5.7-6.5%, 5.8-6.5%, 5.9-6.5%, 6-6.5%, 6.1-6.5%, 6.2-6.5%, 6.3-6.5%, 6.4-6.5%, 0-7%, 0.1-7%, 0.2-7%, 0.3-7%, 0.4-7%, 0.5-7%, 0.6-7%, 0.7-7%, 0.8-7%, 0.9-7%, 1-7%, 1.1-7%, 1.2-7%, 1.3-7%, 1.4-7%, 1.5-7%, 1.6-7%, 1.7-7%, 1.8-7%, 1.9-7%, 2-7%, 2.1-7%, 2.2-7%, 2.3-7%, 2.4-7%, 2.5-7%, 2.6-7%, 2.7-7%, 2.8-7%, 2.9-7%, 3-7%, 3.1-7%, 3.2-7%, 3.3-7%, 3.4-7%, 3.5-7%, 3.6-7%, 3.7-7%, 3.8-7%, 3.9-7%, 4-7%, 4.1-7%, 4.2-7%, 4.3-7%, 4.4-7%, 4.5-7%, 4.6-7%, 4.7-7%, 4.8-7%, 4.9-7%, 5-7%, 5.1-7%, 5.2-7%, 5.3-7%, 5.4-7%, 5.5-7%, 5.6-7%, 5.7-7%, 5.8-7%, 5.9-7%, 6-7%, 6.1-7%, 6.2-7%, 6.3-7%, 6.4-7%, 6.5-7%, 6.6-7%, 6.7-7%, 6.8-7%, 6.9-7%, 0-7.5%, 0.1-7.5%, 0.2-7.5%, 0.3-7.5%, 0.4-7.5%, 0.5-7.5%, 0.6-7.5%, 0.7-7.5%, 0.8-7.5%, 0.9-7.5%, 1-7.5%, 1.1-7.5%, 1.2-7.5%, 1.3-7.5%, 1.4-7.5%, 1.5-7.5%, 1.6-7.5%, 1.7-7.5%, 1.8-7.5%, 1.9-7.5%, 2-7.5%, 2.1-7.5%, 2.2-7.5%, 2.3-7.5%, 2.4-7.5%, 2,5-7.5%, 2.6-7.5%, 2.7-7.5%, 2.8-7.5%, 2.9-7.5%, 3-7.5%, 3.1-7.5%, 3.2-7.5%, 3.3-7.5%, 3.4-7.5%, 3.5-7.5%, 3.6-7.5%, 3.7-7.5%, 3.8-7.5%, 3.9-7.5%, 4-7.5%, 4.1-7.5%, 4.2-7.5%, 4.3-7.5%, 4.4-7.5%, 4.5-7.5%, 4.6-7.5%, 4.7-7.5%, 4.8-7.5%, 4.9-7.5%, 5-7.5%, 5.1-7.5%, 5.2-7.5%, 5.3-7.5%, 5.4-7.5%, 5.5-7.5%, 5.6-7.5%, 5.7-7.5%, 5.8-7.5%, 5.9-7.5%, 6-7.5%, 6.1-7.5%, 6.2-7.5%, 6.3-7.5%, 6.4-7.5%, 6.5-7.5%, 6.6-7.5%, 6.7-7.5%, 6.8-7.5%, 6.9-7.5%, 7-7.5%, 7.1-7.5%, 7.2-7.5%, 7.3-7.5%, 7.4-7.5%, 0-8%, 0.1-8%, 0.2-8%, 0.3-8%, 0.4-8%, 0.5-8%, 0.6-8%, 0.7-8%, 0.8-8%, 0.9-8%, 1-8%, 1.1-8%, 1.2-8%, 1,3-8%, 1.4-8%, 1.5-8%, 1.6-8%, 1.7-8%, 1.8-8%, 1.9-8%, 2-8%, 2.1-8%, 2.2-8%, 2.3-8%, 2.4-8%, 2.5-8%, 2.6-8%, 2.7-8%, 2.8-8%, 2.9-8%, 3-8%, 3.1-8%, 3.2-8%, 3.3-8%, 3.4-8%, 3.5-8%, 3.6-8%, 3.7-8%, 3.8-8%, 3.9-8%, 4-8%, 4.1-8%, 4.2-8%, 4.3-8%, 4.4-8%, 4,5-8%, 4.6-8%, 4.7-8%, 4.8-8%, 4.9-8%, 5-8%, 5.1-8%, 5.2-8%, 5.3-8%, 5.4-8%, 5.5-8%, 5.6-8%, 5.7-8%, 5.8-8%, 5.9-8%, 6-8%, 6.1-8%, 6.2-8%, 6.3-8%, 6.4-8%, 6.5-8%, 6.6-8%, 6.7-8%, 6.8-8%, 6.9-8%, 7-8%, 7.1-8%, 7.2-8%, 7.3-8%, 7.4-8%, 7.5-8%, 7.6-8%, 7.7-8%, 7.8-8%, 7.9-8%, 0-8.5%, 0.1-8.5%, 0.3-8.5%, 0.4-8.5%, 0.5-8.5%, 0.6-8.5%, 0.7-8.5%, 0.8-8.5%, 0.9-8.5%, 1-8.5%, 1.1-8.5%, 1.2-8.5%, 1.3-8.5%, 1.4-8.5%, 1.5-8.5%, 1.6-8.5%, 1.7-8.5%, 1.8-8.5%, 1.9-8.5%, 2-8.5%, 2.1-8.5%, 2.2-8.5%, 2.3-8.5%.2.4-8.5%. 2.5-8.5%, 2.6-8.5%, 2.7-8.5%, 2.8-8.5%, 2.9-8.5%, 3-8.5%, 3.1-8.5%, 3.2-8.5%, 3.3-8.5%, 3.4-8.5%, 3.5-8.5%, 3.6-8.5%, 3.7-8.5%, 3.8-8.5%, 3.9-8.5%, 4-8.5%, 4.1-8.5%, 4.2-8.5%, 4.3-8.5%, 4.4-8.5%, 4.5-8.5%, 4.6-8.5%, 4.7-8.5%, 4.8-8.5%, 4.9-8.5%, 5-8.5%, 5.1-8.5%, 5.2-8.5%, 5.3-8.5%, 5.4-8.5%, 5.5-8.5%, 5.6-8.5%, 5.7-8.5%, 5.8-8.5%, 5.9-8.5%, 6-8.5%, 6.1-8.5%, 6.2-8.5%, 6.3-8.5%, 6.4-8.5%, 6.5-8.5%, 6.6-8.5%, 6.7-8.5%, 6.8-8.5%, 6.9-8.5%, 7-8.5%, 7.1-8.5%, 7.2-8.5%, 7.3-8.5%, 7.4-8.5%, 7.5-8.5%, 7.6-8.5%, 7.7-8.5%, 7.8-8.5%, 7.9-8.5%, 8-8.5%, 8.1-8.5%, 8.2-8.5%, 8.3-8.5%, 8.4-8.5%, 0-9%, 0.1-9%, 0.2-9%, 0.3-9%, 0.4-9%, 0.5-9%, 0.6-9%, 0.7-9%, 0.8-9%, 0.9-9%, 1-9%, 1.1-9%, 1.2-9%, 1.3-9%, 1.4-9%, 1.5-9%, 1.6-9%, 1.7-9%, 1.8-9%, 1.9-9%, 2-9%. 2.1-9%, 2.2-9%, 2.3-9%, 2.4-9%, 2.5-9%, 2.6-9%, 2.7-9%, 2.8-9%, 2.9-9%, 3-9%, 3.1-9%, 3.2-9%, 3.3-9%, 3.4-9%, 3.5-9%, 3.6-9%, 3.7-9%, 3.8-9%, 3.9-9%, 4-9%, 4.1-9%, 4.2-9%, 4.3-9%, 4.4-9%, 4.5-9%, 4.6-9%, 4.7-9%, 4.8-9%, 4.9-9%, 5-9%, 5.1-9%, 5.2-9%, 5.3-9%, 5.4-9%, 5.5-9%, 5.6-9%, 5.7-9%, 5.8-9%, 5.9-9%, 6-9%, 6.1-9%, 6.2-9%, 6.3-9%, 6.4-9%, 6.5-9%, 6.6-9%, 6.7-9%, 6.8-9%, 6.9-9%, 7-9%, 7.1-9%, 7.2-9%, 7.3-9%, 7.4-9%, 7.5-9%, 7.6-9%, 7.7-9%, 7.8-9%, 7.9-9%, 8-9%, 8.1-9%, 8.2-9%, 8.3-9%, 8.4-9%, 8.5-9%, 8.6-9%, 8.7-9%, 8.8-9%, 8.9-9%, 0-9.5%, 0.1-9.5%, 0.2-9.5%, 0.3-9.5%, 0.4-9.5%, 0.5-9.5%, 0.6-9.5%, 0.7-9.5%, 0.8-9.5%, 0.9-9.5%, 1-9.5%, 1.1-9.5%, 1.2-9.5%, 1.3-9.5%, 1.4-9.5%, 1.5-9.5%, 1.6-9.5%, 1.7-9.5%, 1.8-9.5%, 1.9-9.5%, 2-9.5%, 2.1-9.5%, 2.2-9.5%, 2.3-9.5%, 2.4-9.5%, 2.5-9.5%, 2.6-9.5%, 2.7-9.5%, 2.8-9.5%, 2.9-9.5%, 3-9.5%, 3.1-9.5%, 3.2-9.5%, 3.3-9.5%, 3.4-9.5%, 3.5-9.5%, 3.6-9.5%, 3.7-9.5%, 3.8-9.5%, 3.9-9.5%, 4-9.5%, 4.1-9.5%, 4.2-9.5%, 4.3-9.5%, 4.4-9.5%, 4.5-9.5%, 4.6-9.5%, 4.7-9.5%, 4.8-9.5%, 4.9-9.5%, 5-9.5%, 5.1-9.5%, 5.2-9.5%, 5.3-9.5%, 5.4-9.5%, 5.5-9.5%, 5.6-9.5%, 5.7-9.5%, 5.8-9.5%, 5.9-9.5%, 6-9.5%, 6.1-9.5%, 6.2-9.5%, 6.3-9.5%, 6.4-9.5%, 6.5-9.5%, 6.6-9.5%, 6.7-9.5%, 6.8-9.5%, 6.9-9.5%, 7-9.5%, 7.1-9.5%, 7.2-9.5%, 7.3-9.5%, 7.4-9.5%, 7.5-9.5%, 7.6-9.5%, 7.7-9.5%, 7.8-9.5%, 7.9-9.5%, 8-9.5%, 8.1-9.5%, 8.2-9.5%, 8.3-9.5%, 8.4-9.5%, 8.5-9.5%, 8.6-9.5%, 8.7-9.5%, 8.8-9.5%, 8.9-9.5%, 9-9.5%, 9.1-9.5%, 9.2-9.5%, 9.3-9.5%, 9.4-9.5%, 0-10%, 0.1-10%, 0.2-10%, 0.3-10%, 0.4-10%, 0.5-10%, 0.6-10%, 0.7-10%, 0.8-10%, 0.9-10%, 1-10%, 1.1-10%, 1.2-10%, 1.3-10%, 1.4-10%, 1.5-10%, 1.6-10%, 1.7-10%, 1.8-10%, 1.9-10%, 2-10%, 2.1-10%, 2.2-10%, 2.3-10%, 2.4-10%, 2.5-10%, 2.6-10%, 2.7-10%, 2.8-10%, 2.9-10%, 3-10%, 3.1-10%, 3.2-10%, 3.3-10%, 3.4-10%, 3.5-10%, 3.6-10%, 3.7-10%, 3.8-10%, 3.9-10%, 4-10%, 4.1-10%, 4.2-10%, 4.3-10%, 4.4-10%, 4.5-10%, 4.6-10%, 4.7-10%, 4.8-10%, 4.9-10%, 5-10%, 5.1-10%, 5.2-10%, 5.3-10%, 5.4-10%, 5.5-10%, 5.6-10%, 5.7-10%, 5.8-10%, 5.9-10%, 6-10%, 6.1-10%, 6.2-10%, 6.3-10%, 6.4-10%, 6.5-10%, 6.6-10%, 6.7-10%, 6.8-10%, 6.9-10%, 7-10%, 7.1-10%, 7.2-10%, 7.3-10%, 7.4-10%, 7.5-10%, 7.6-10%, 7.7-10%, 7.8-10%, 7.9-10%, 8-10%, 8.1-10%, 8.2-10%, 8.3-10%, 8.4-10%, 8.5-10%, 8.6-10%, 8.7-10%, 8.8-10%, 8.9-10%, 9-10%, 9.1-10%, 9.2-I0%, 9.3-10%, 9.4-10%, 9.5-10%, 9.6-10%, 9.7-10%, 9.8-10%, or 9.9-10% w/v.

In certain embodiments, the formulation may comprise 0-10% w/v of sorbitol.

In certain embodiments, the formulation may comprise 0-9% w/v of sorbitol.

In certain embodiments, the formulation may comprise 0-8% w/v of sorbitol.

In certain embodiments, the formulation may comprise 0-7% w/v of sorbitol.

In certain embodiments, the formulation may comprise 0-6% w/v of sorbitol.

In certain embodiments, the formulation may comprise 0-5% w/v of sorbitol.

In certain embodiments, the formulation may comprise 0-4% w/v of sorbitol.

In certain embodiments, the formulation may comprise 0-3% w/v of sorbitol.

In certain embodiments, the formulation may comprise 0-2% w/v of sorbitol.

In certain embodiments, the formulation may comprise 0-1% w/v of sorbitol.

In certain embodiments, the formulation may comprise 1% w/v of sorbitol.

In certain embodiments, the formulation may comprise 2% w/v of sorbitol.

In certain embodiments, the formulation may comprise 3% w/v of sorbitol,

In certain embodiments, the formulation may comprise 4% w/v of sorbitol.

In certain embodiments, the formulation may comprise 5% w/v of sorbitol.

In certain embodiments, the formulation may comprise 6% w/v of sorbitol.

In certain embodiments, the formulation may comprise 7% w/v of sorbitol.

In certain embodiments, the formulation may comprise 8% w/v of sorbitol.

In certain embodiments, the formulation may comprise 9% w/v of sorbitol.

In certain embodiments, the formulation may comprise 10% w/v of sorbitol.

### Surfactant

In certain embodiments, formulations of pharmaceutical compositions described herein may comprise a surfactant. Surfactants may help control shear threes in suspension cultures. Surfactants used herein may be anionic, zwitterionic, or non-ionic surfactants and may comprise those known in the art that are suitable for use in pharmaceutical formulations.

Examples of anionic surfactants comprise, but are not limited to. sulfate, sulthnate, phosphate esters, and carboxylates.

Examples of nonionic surfactants comprise, but are not limited to, ethoxylates, fatty alcohol ethoxylates, alkylphenol ethoxylates (e.g., nonoxynols, Triton X-100), fatty acid ethoxylates, ethoxylated amines and/or fatty acid amides (e.g., polyethoxylated tallow amine, cocamide monoethanolamine, cocamide diethanolamine), ethylene oxide/propylene oxide copolymer (e.g., Poloxamers such as Pluronic® F-68 or F-127), esters of fatty acids and polyhydric alcohols, fatty acid alkanolamides, ethoxylated aliphatic acids, ethoxylated aliphatic alcohols, ethoxylated sorbitol fatty acid esters, ethoxylated glycerides, ethoxylated block copolymers with EDTA (ethylene diaminetetraacetie acid), ethoxylated cyclic ether adducts, ethoxylated amide and imidazoline adducts, ethoxylated amine adducts, ethoxylated mercaptan adducts, ethoxylated condensates with alkyl phenols, ethoxylated nitrogen-based hydrophobes, ethoxylated polyoxypropylenes, polymeric silicones, fluorinated surfactants, and polymerizable surfactants.

Examples of zwitterionic surfactants comprise, but are not limited to, alkylamido betaines and amine oxides thereof, alkyl betaines and amine oxides thereof, sulfo betaines, hydroxy sulfo betaines, amphoglycinates, amphopropionates, balanced amphopoly-carboxyglycinates, and alkyl polyaminoglycinates. Proteins have the ability of being charged or uncharged depending on the pH; thus, at the right pH, a protein, preferably with a pI of about 8 to 9, such as modified Bovine Serum Albumin or chymotrypsinogen, could function as a zwitterionic surfactant. Various mixtures of surfactants can be used if desired,

### Copolymers

In certain embodiments, at least one of the components in the formulation is copolymer.

In certain embodiments, the formulation may comprise at least one copolymer at a concentration of 0.00001%, 0.0001%, 0.001%, 0.01%, 0.1%, or 1% w/v.

In certain embodiments, the formulation may comprise at least one copolymer in a range of 0,00001%-0.0001%, 0.00001%-0.001%, 0.00001%4).01%, 0.00001%-0.1%, 0.00001%-1%, 0.0001%-0.001%, 0.0001%-0.01%, 0.0001%-0.1%; 0.0001%-1%; 0.001%-0.01%, 0.001%41%, 0.001%-1%, 0.01%-0.1%, 0.01%-1%, or 0.1-1% w/v.

In certain embodiments, the formulation may comprise 0.001% w/v copolymer.

In certain embodiments, the copolymer is an ethylene oxide/propylene oxide copolymer.

In certain embodiments, the formulation may comprise at least one ethylene oxide/propylene oxide copolymer at a concentration of 0.00001%, 0.0001%, 0.001%, 0.01%, 0.1%; or 1% w/v.

In certain embodiments, the formulation may comprise at least one ethylene oxide/propylene oxide copolymer in a range of 0,00001%-0.0001%, 0.00001%-0.001%, 0.00001%-0.01%, 0.00001%-0.1%, 0.00001%-1%, 0.0001%-0.001%, 0.0001%-0.01%, 0.0001%-0.1%, 0.0001%-1%, 0.001%-0.01%. 0.001%-0.1%, 0.001%-1%, 0.01%-0.1%, 0.01%4%, or 0.1-1% w/v.

In certain embodiments, the formulation may comprise 0.001% w/v ethylene oxide/propylene oxide copolymer.

In certain embodiments, the formulation may comprise at least one ethylene oxide/propylene copolymer which is a Poloxamer. In certain embodiments, the formulation may comprise Poloxamer at a concentration of 0.00001%. 0.0001%, 0.001%, 0.01%, 0.1%, or 1% w/v.

In certain embodiments, the formulation may comprise Poloxamer in a range of 0.00001%-0.0001%, 0.00001%-0.001%, 0.00001%-0.01%, 0.00001%-0.1%, 0.00001%-1%, 0.0001%-0.001%, 0.0001%-0.01%, 0.0001%-0.1%, 0.0001%-1%, 0.001%-0.01%, 0.001%-0.1%, 0.001%-1%, 0.01%-0.1%, 0.01%-1%, or 0.1-1% w/v.

In certain embodiments, the formulation may comprise 0.001% w/v Poloxamer.

In certain embodiments, the formulation may comprise at least one ethylene oxide/propylene copolymer which is Poloxamer 188. In certain embodiments, the formulation may comprise Poloxamer 188 at a concentration of 0.00001%, 0.0001%, 0.001%, 0.01%, 0.1%, or 1% w/v.

In certain embodiments, the formulation may comprise Poloxamer 188 in a range of 0.00001%-0.0001%, 0.00001%-0.001%, 0.00001%-0.01%, 0.00001%-0.1%, 0.00001%-1%, 0.0001%-0.001%, 0.0001%-0.01%, 0.0001%-0.1%, 0.0001%-1%, 0.001%-0.01%, 0.001%-0.1%, 0.001%-1%, 0.01%-0.1%, 0.01%-1%, or 0.1-1% w/v.

In certain embodiments, the formulation may comprise 0.001%-0.1 w/v Poloxamer 188.

In certain embodiments, the formulation may comprise at least one ethylene oxide/propylene, copolymer which is Pluronic F-68. In certain embodiments, the formulation may comprise Pluronic® F-68 at a concentration of 0.00001%, 0.0001%, 0.001%, 0.01%, 0.1%, or 1% w/v.

In certain embodiments, the formulation may comprise Pluronic® F-68 in a range of 0.00001%-0.0001%, 0.00001%-0.001%, 0.00001%-0.01%, 0.00001%-0.1%, 0.00001%-1%, 0.0001%-0.001%, 0.0001%-0.01%, 0.0001%-0.1%, 0.0001%-1%, 0.001%-0.01%, 0.001%-0.1%, 0.001%-1%, 0.01%-0.1%, 0.01%-1%, or 0.1-1% w/v.

In certain embodiments, the formulation may comprise 0,001%-0.1% w/v Pluronic® F-68. In certain embodiments, the formulation may comprise 0.001% w/v Pluronic® F-68.

### Formulation Properties

In certain embodiments, the formulation has been optimized to have a specific pH, osmolality, concentration, concentration of AAV particle, and/or total dose of AAV particle.

pH

In certain embodiments, the formulation may be optimized for a specific pH. In certain embodiments, the formulation may comprise a pH buffering agent (also referred to herein as “buffering agent”) which is a weak acid or base that, when used in the formulation, maintains the pH of the formulation near a chosen value even after another acid or base is added to the formulation. The pH of the formulation may be, but is not limited, to 0. 0.1, 0.2, 0.3. 0.4. 0.5. 0.6. 0.7. 0.8. 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11,6, 11,7, 11.8, 11.9, 12, 12,1, 12,2, 12 12.4, 12.5, 12.6, 12.7, 12.8, 12.9, 13, 13.1, 13.2, 13.3, 13.4, 13.5, 13.6, 13.7, 13.8, 13.9, and 14.

In certain embodiments, the formulation may be optimized for a specific pH range. The pH range may he, but is not limited to, 0-4, 1-5, 2-6, 3-7, 4-8, 5-9, 6-10, 7-11, 8-12, 9-13, 10-14, 0-1.5, 1-2.5, 2-3.5, 3-4.5, 4-5.5, 5-6.5, 6-7.5, 7-8.5, 8-9.5, 9-10.5, 10-11.5, 11-12.5, 12-13.5, 0-1, 1-2, 2-3, 3-4, 4-5, 5-6, 6-7, 7-8, 8-9, 9-10, 10-11, 11-12, 12-13, 13-14, 0-0.5, 0.5-1, 1-1.5, 1.5-2, 2-2.5, 2.5-3, 3-3.5, 3.5-4, 4-4.5, 4.5-5, 5-5.5, 5.5-6, 6-6.5, 6.5-7, 7-7.5, 7.2-8.2, 7.2-7.6, 7.3-7.7, 7.5-8, 7.8-8.2, 8-8.5, 8.5-9, 9-9.5, 9.5-10, 10-10.5, 10.5-11, 11-11.5, 11.5-12, 12-12.5, 12.5-13, 13-13.5, or 13.5-14.

In certain embodiments, the pH of the formulation is between 6 and 8.5.

In certain embodiments, the pH of the formulation is between 7 and 8.5

In certain embodiments, the pH of the formulation is between 7 and 7.6.

In certain embodiments, the pH of the formulation is 7.

In certain embodiments, the pH of the formulation is 7.1.

In certain embodiments, the pH of the formulation is 7.2.

In certain embodiments, the pH of the formulation is 7.3.

In certain embodiments, the pH of the formulation is 7.4.

In certain embodiments, the pH of the formulation is 7.5.

In certain embodiments, the pH of the formulation is 7.6.

In certain embodiments, the pH of the formulation is 7.7.

In certain embodiments, the pH of the formulation is 7.8.

In certain embodiments, the pH of the formulation is 7.9.

In certain embodiments, the pH of the formulation is 8.

In certain embodiments, the pH of the formulation is 8.1.

In certain embodiments, the pH of the formulation is 8.2.

In certain embodiments, the pH of the formulation is 8.3.

In certain embodiments, the pH of the formulation is 8.4.

In certain embodiments, the pH of the formulation is 8.5.

In certain embodiments, the pH is determined when the formulation is at 5° C.

In certain embodiments, the pH is determined when the formulation is at 25° C.

Suitable buffering agents may comprise, but not limited to, Tris HCl, Tris base, sodium phosphate (monosodium phosphate and/or disodium phosphate), potassium phosphate (monopotassium phosphate and/or dipotassimn phosphate), histidine, boric acid, citric acid, glycine, HEPES (4-(2-hydroxyethyl)-1-piperazineethanesulfonic acid), and MOPS (3(N-morpholino)propariesulfonic acid).

Concentration of buffering agents in the formulation may be between 1-50 mM, between 1-25 mM, between 5-30 mM, between 5-20 mM, between 5-15 mM, between 10-40 mM, or between 15-30 mM. Concentration of buffering agents in the formulation may be about 1 mM, 5 mM, 7.5 mM, 10 mM, 12.5 mM, 15 mM, 20 mM, 25 mM, 30 mM, 35 mM, 40 mM, or 50 mM.

In certain embodiments, the formulation may comprise, but is not limited to, phosphate-buffered saline (PBS). As a non-limiting example, the PBS may comprise sodium chloride, potassium chloride, disodium phosphate, monopotassium phosphate, and distilled water. in some instances, the PBS does not contain potassium or magnesium. In other instances, the PBS contains calcium and magnesium.

In certain embodiments, buffering agents used in the formulations of pharmaceutical compositions described herein may comprise sodium phosphate (monosodium phosphate and/or disodium phosphate). As a non-limiting example, sodium phosphate may be adjusted to a pH (at 5° C.) within the range of 7.4±0.2. In certain embodiments, buffering agents used in the formulations of pharmaceutical compositions described herein may comprise Tris base. Tris base may be adjusted with hydrochloric acid to any pH within the range of 7.1 and 9.1. As a non-limiting example, Tris base used in the formulations described herein may be adjusted to 8.0±0.2. As a non-limiting example, Tris base used in the formulations described herein may be adjusted to 7.5±0.2.

### Osmolality

In certain embodiments, the formulation may be optimized for a specific osmolality. The osmolality of the formulation may be, but is not limited to, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 mOsm/kg (milliosmoles/kg).

In certain embodiments, the formulation may be optimized for a specific range of osmolality. The range may be, but is not limited to, 350-360, 360-370, 370-380, 380-390, 390-400, 400-410, 410-420, 420-430, 430-440, 440-450, 450-460, 460-470, 470-480, 480-490, 490-500, 350-370, 360-380, 370-390, 380-400, 390-410, 400-420, 410-430, 420-440, 430-450, 440-460, 450-470, 460-480, 470-490, 480-500, 350-375, 375-400, 400-425, 425-450, 450-475, 475-500, 350-380, 360-390, 370-400, 380-410, 390-420, 400-430, 410-440, 420-450, 430-460, 440-470, 450-480, 460-490, 470-500, 350-390, 360-400, 370-410, 380-420, 390-430, 400-440, 410-450, 420-460, 430-470, 440-480, 450-490, 460-500, 350-400, 360-410, 370-420, 380-430, 390-440, 400-450, 410-460, 420-470, 430-480, 440-490, 450-500, 350-410, 360-420, 370-430, 380-440, 390-450, 400-460, 410-470, 420-480, 430-490, 440-500, 350-420, 360-430, 370-440, 380-450, 390-460, 400-470, 410-480, 420-490, 430-500, 350-430, 360-440, 370-450, 380-460, 390-470, 400-480, 410-490, 420-500, 350-440, 360-450, 370-460, 380-470, 390-480, 400-490, 410-500, 350-450, 360-460, 370-470, 380-480, 390-490, 400-500, 350-460, 360-470, 370-480, 380-490, 390-500, 350-470, 360-480, 370-490, 380-500, 350-480, 360-490, 370-500, 350-490, 360-500, or 350-500 mOs/kg.

In certain embodiments, the osmolality of the formulation is between 350-500 mOsm/kg.

In certain embodiments, the osmolality of the formulation is between 400-500 mOsm/kg.

In certain embodiments, the osmolality of the formulation is between 400-480 mOsm/kg.

In certain embodiments, the osmolality is 395 mOsm/kg.

In certain embodiments, the osmolality is 413 mOsm/kg.

In certain embodiments, the osmolality is 420 mOsm/kg.

In certain embodiments, the osmolality is 432 mOsm/kg.

In certain embodiments, the osmolality is 447 mOsm/kg,

In certain embodiments, the osmolality is 450 mOsm/kg.

In certain embodiments, the osmolality is 452 mOsm/kg.

In certain embodiments, the osmolality is 459 mOsm/kg.

In certain embodiments, the osmolality is 472 mOsm/kg.

In certain embodiments, the osmolality is 490 mOsm/kg.

In certain embodiments, the osmolality is 496 mOsm/kg.

### Concentration of AAV Particle

In certain embodiments, the concentration of AAV particle in the formulation may be between about 1×10⁶ VG/mi and about 1×10¹⁶ VG/ml. As used herein, “VG/ml” represents vector genomes (VG) per milliliter (ml). VG/ml also may describe genome copy per milliliter or DNase resistant particle per milliliter.

In certain embodiments, the formulation may comprise an AAV particle concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2,4×10¹¹, 2.5×10¹¹, 2.6×10¹¹, 2.7×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1.2×10¹², 1.3×10¹², 1.4×10¹², 1.5×10¹¹, 1.6×10¹², 1.7×10¹², 1.8×10¹², 1.9×10¹², 2×10¹², 2.1×10¹², 2.2×10¹², 2.3×10¹², 2.4×1.0¹², 2.5×10¹², 2.6×10¹², 2.7×10¹², 2.8×10¹², 2.9×10¹², 3×1.0¹¹, 4×10¹², 4.1×10¹², 4.2×10¹², 4.3×10¹², 4.4×10¹², 4.5×10¹², 4.6×10¹², 4.7×10¹¹, 4.8×10¹², 4.9×10¹², 5×10¹², 6×1.0¹², 7×10¹², 7.1×10¹², 7.2×10¹², 7.3×10¹², 7.4×10¹², 7.5×10¹¹, 7.6×10¹², 7.7×10¹², 7.8×10¹², 7.9×10¹², 8×10¹², 8.1×10¹², 8.2×10¹², 8.3×10¹², 8.4×10¹², 8.5×10¹², 8.6×10¹², 8.7×10¹², 8.8×10¹², 8.9×10¹², 9×10¹², 1×10^{—}, 1.1×10¹³, 1.2×10¹³, 1.3×10¹³, 1.4×10¹³, 1.5×10¹³, 1.6×10¹³, 1.7×10¹³, 1.8×10¹³ , 1.9×10¹³, 2×10¹³, 2.1×10¹³, 2.2×10¹³, 2.3×10¹³, 2.4×10¹³ , 2.5×10¹³, 2.6×10¹³, 2.7×10¹³, 2.8×10¹³, 2.9×10¹³, 3×10¹³, 3.1×10¹³, 3.2×10¹³, 3.3×10¹³, 3.4×10¹³, 3.5×10¹³, 3.6×10¹³, 3.7×10¹³, 3.8×10¹³, 3.9×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/ml.

In certain embodiments, the concentration of AAV particle in the formulation is between 1×10¹¹ and 5×10¹³, between 1×10¹² and 5×10¹², between 2×10¹² and 1×10¹³, between 5×10¹² and 1×10¹³, between 1×10¹³ and 2×10¹³, between 2 ×10¹³ and 3 ×10¹³, between 2×10¹³ and 2.5×10¹³, between 2.5×10¹³ and 3×10¹³, or no more than 5×10¹³ VG/ml.

In certain embodiments, the concentration of AAV particle in the formulation s 2.7×10¹¹ VG/ml.

In certain embodiments, the concentration of AAV particle in the formulation is 9×10¹¹ VG/ml.

In certain embodiments, the concentration of AAV particle in the formulation is 1.22×10¹² VG/ml.

In certain embodiments, the concentration of AAV particle in the formulation is 2.7×10¹² VG/ml.

In certain embodiments, the concentration of AAV particle in the formulation is 4×10¹² VG/ml.

In certain embodiments, the concentration of AAV particle in the formulation is 6×10¹² VG/ml.

In certain embodiments, the concentration of AAV particle in the formulation is 7.9×10¹² VG/ml.

In certain embodiments, the concentration of AAV particle in the formulations 8×10¹² VG/ml.

In certain embodiments, the concentration of AAV particle in the formulation is 1×10¹³ VG/ml.

In certain embodiments, the concentration of AAV particle in the formulation is 1.8×10¹³ VG/ml.

In certain embodiments, the concentration of AAV particle in the formulation is 2.2×10¹³ VG/ml.

In certain embodiments, the concentration of AAV particle in the formulation is 2.7×10¹³ VG/ml.

In certain embodiments, the concentration of AAV particle in the formulation is 3.5×10¹³ VG/ml.

In certain embodiments, the concentration of AAV particle in the formulation is 2.7-3.5×10¹⁶ VG/ml.

In certain embodiments, the concentration of AAV particle in the formulation is 7.0×10¹³ VG/ml.

In certain embodiments, the concentration of. particle in the formulation is 5.0×10¹² VG/ml.

In certain embodiments, the concentration of AAV particle in the formulation may be between about 1×10⁶ total capsid/mL and about 1×10¹⁶ total capsid/ml, in certain embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×1.0⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹⁰, 2.1×10¹⁷, 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.23×10¹², 33×10¹², 3.4×10¹², 3.5×10¹², 3.6×10¹², 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 2.1×10¹³, 2.2×10¹³, 2.3×10¹³, 2.4×10¹³, 2.5×10¹³, 2.6×10¹³ , 2.7×10¹³, 7.8×10¹³, 2.9×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹¹, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ total capsid/ml.

### Total Dose of AAV Particle

In certain embodiments, the total dose of the AAV particle in the formulation may be between about 1×10⁶ VG and about 1×10¹⁶ VG. In certain embodiments, the formulation may comprise a total dose of AAV particle of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10³, 6×10³, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 10×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 2.1×10¹¹, 2,2×10¹¹, 2.3×10¹¹, 2.4×1.0¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1 ×10¹², 1.2×10¹², 1.3×10¹², 1.4×10¹², 1.5×10¹², 1.6×10¹², 1.7×10′², 1.8×10¹², 1.9×10¹², 2×10¹², 2.1×10¹², 2.2×10¹², 2.3×1.0¹², 2.4×10¹², 2.5×10¹², 2.6×10¹², 2.7×10¹³, 2.8×10¹², 2.9×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 7.1×10¹², 7.2×10¹², 7.3×10¹², 7.4×10¹², 7.5×10¹², 7.6×10¹², 7.7×10¹², 7.8×10¹², 7.9×10¹², 8×10¹², 8.1×10¹², 8.2×10¹², 8.3×10¹², 8.4×10¹², 8.5×10¹², 8.6×10¹², 8,7×10¹², 8.8 ×10¹², 8.9×10¹², 9×10¹², 1×10′³, 1.1×1.0¹³, 1.2×10¹³, 1.3×10¹³, 1.4×1.0¹³, 1.5×10¹³, 1.6×10¹³, 1.7×10¹³, 1.8×10¹³, 1.9×10¹³, 2×10¹³, 2.1×10¹³, 2.2×10¹³, 2.3×10¹³, 2.4×10¹³, 2.5×10′³, 2.6×10¹³, 2.7×10¹³, 2.8×10¹³, 2.9×10¹³, 3×10¹³, 3.1×10¹³, 3.2×10¹³, 3.3×10¹³, 3.4×10¹³, 3.5×10¹³, 3.6×1.0¹³, 3,7×10¹³, 3.8×10¹³, 3.9×10¹³, 4×10¹³, 5×10¹³, 6×10¹³, 6.7×10¹³, 7×10¹³, 8×10¹³, 9×10¹³, 1×2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10′⁵, 6×10¹⁵, 7×10′⁵, 8×10¹⁵, 9×10¹⁵, or 1×10¹⁶ VG.

In certain embodiments, the total dose of., V particle in the formulation between 1×10¹¹ and 5×10¹³ VG.

In certain embodiments, the total dose of AAV particle in the formulation is between 1×10¹¹ and 2×10¹⁴ VG.

In certain embodiments, the total dose of AAV particle in the formulation is 1.4×10¹¹ VG.

In certain embodiments, the total dose of AAV particle in the formulation is 4.5×10¹¹ VG.

In certain embodiments, the total dose of AAV particle in the formulation is 6.8×10¹¹ VG.

In certain embodiments, the total dose of AA particle in the formulation is 1.4×10¹² VG.

In certain embodiments, the total dose of AAV particle in the formulation is 2.2×10¹² VG.

In certain embodiments, the total dose of AAV particle in the formulation is 4.6×10¹¹ VG.

In certain embodiments, the total dose of AAV particle in the formulation is 9.2×10¹² VG.

In certain embodiments, the total dose of AAV particle in the formulation is 1.0×10¹³ VG.

In certain embodiments, the total dose of AAV particle in the formulation is 2.3×10¹³ VG.

### Exemplary Formulations

Described below are exemplary, non-limiting formulations of the present disclosure. The formulations may comprise AAV-particle formulations. Table 7 presents a summary of the components and properties of certain exemplary formulations of the present disclosure. Each formulation may optionally comprise 0.001%-0.1% (w/v) of Poloxamer 188 (e.g. Pluronic F-68®).

TABLE 7
Exemplary Formulations

| | Sodium | Potassium | Sodium | Potassium | | | | |
|---|---|---|---|---|---|---|---|---|
| Formulation | phosphate | phosphate | chloride | chloride | Sugar | Other | | Osmolality |
| ID. | (mM) | (mM) | (mM) | (mM) | (w/v) | (mM) | pH | (mOsm/kg) |
| VYFORM1 | 10 | 1.5 | 95 | — | 7% | — | 7.4 | — |
| | | | | | (S) | | | |
| VYFORM2 | 2.7 | 1.5 | 155 | — | 5% | — | 7.2 | 450 |
| | | | | | (S) | | | |
| VYFORM3 | 2.7 | 1.5 | 107 | — | 7% | — | 6.9 | 428 |
| | | | | | (S) | | | |
| VYFORM4 | 2.7 | 1.5 | 92 | — | 7% | — | 6.9 | 402 |
| | | | | | (S) | | | |
| VYFORM5 | 2.7 | 1.5 | 98 | — | 9% | — | 6.9 | 428 |
| | | | | | (S) | | | |
| VYFORM6 | 2.7 | 1.5 | 83 | — | 9% | — | 6.9 | 402 |
| | | | | | (S) | | | |
| VYFORM7 | 2.7 | 1.5 | 150 | — | 7% | — | — | — |
| | | | | | (S) | | | |
| VYFORM8 | 2.7 | 1.5 | 150 | — | 9% | — | — | — |
| | | | | | (S) | | | |
| VYFORM9 | 10 | 2 | 192 | 2.7 | 1% | — | 7.4 | — |
| | | | | | (S) | | | |
| VYFORM10 | 10 | 2 | 150 | 2.7 | 3% | — | — | — |
| | | | | | (S) | | | |
| VYFORM11 | 10 | 2 | 125 | 2.7 | 5% | — | — | — |
| | | | | | (T) | | | |
| VYFORM12 | — | 2 | 125 | 2.7 | 5% | 10 (His) | — | — |
| VYFORM13 | — | — | 142 | 1.5 | 5% | 10 (Tris) | 7.4 | 424 |
| | | | | | (S) | | | |
| VYFORM14 | — | — | 127 | 1.5 | 5% | 10 (Tris) | 7.4 | 404 |
| | | | | | (S) | | | |
| VYFORM15 | — | — | 133 | 1.5 | 7% | 10 (Tris) | 7.4 | 432 |
| | | | | | (S) | | | |
| VYFORM16 | — | — | 118 | 1.5 | 7% | 10 (Tris) | 7.4 | 413 |
| | | | | | (S) | | | |
| VYFORM17 | — | — | 127 | 1.5 | 9% | 10 (Tris) | 7.4 | 436 |
| | | | | | (S) | | | |
| VYFORM18 | — | — | 109 | 1.5 | 9% | 10 (Tris) | 7.4 | 410 |
| | | | | | (S) | | | |
| VYFORM19 | — | — | 100 | 1.5 | 7% | 10 (Tris); | 8.0 | — |
| | | | | | (S) | 6.3 (HCl) | | |
| VYFORM20 | — | — | 100 | 1.5 | 7% | 10 (Tris); | 7.5 | — |
| | | | | | (S) | 9 (HCl) | | |
| VYFORM21 | — | — | 75 | — | 5% | 10 (Tris) | — | — |
| | | | | | (S) | | | |
| VYFORM22 | — | — | 150 | — | 5% | 10 (Tris) | — | — |
| | | | | | (S) | | | |
| VYFORM23 | — | — | 150 | — | 5% | 10 (Tris); | — | — |
| | | | | | (S) | 10 (MgCl₂) | | |
| VYFORM24 | — | — | 75 | — | 5% | 10 (Tris); | — | — |
| | | | | | (S) | 75 (Arg) | | |
| VYFORM25 | — | — | 150 | — | 5% | 10 (Tris) | — | — |
| | | | | | (So) | | | |
| VYFORM26 | — | — | 150 | — | 5% | 10 (His) | — | — |
| | | | | | (S) | | | |
| VYFORM27 | — | 1.5 | — | — | 7% | 10 (Tris) | 8.0 | — |
| | | | | | (S) | | | |
| VYFORM28 | — | — | — | 75 | 5% | 10 (Tris) | — | — |
| | | | | | (S) | | | |
| VYFORM29 | 10 | — | 180 | — | — | — | | |

S = Sucrose (sugar)
T = Trehalose (sugar)
So = Sorbitol (sugar alcohol)
His = Histidine (other)
Tris = tris(hydroxymethyl)aminomethane (other)
Arg = Arginine (other)

In certain embodiments, the formulation may comprise sodium phosphate, potassium phosphate, sodium chloride, sucrose, and optionally a copolymer such as Poloxamer 188 (e.g. Pluronic F-68). In certain embodiments, the formulation may comprise 10 mM sodium phosphate, 1.5 mM potassium phosphate, 100 mM sodium chloride, 5% w/v Sucrose, and optionally Poloxamer 188 (buffer pH of 7.5). In certain embodiments, the formulation may comprise 10 mM sodium phosphate, 1.5 mM potassium phosphate, 220 mM sodium chloride, 5% w/v Sucrose, and optionally Poloxamer 188 (buffer pH of 7.5). In certain embodiments, the formulation may comprise 10 mM sodium phosphate, 1.5 mM potassium phosphate, 100 sodium chloride, 7% w/v Sucrose, and optionally Poloxamer 188 (buffer pH of 7.5).

In certain embodiments, the formulation may comprise sodium phosphate, potassium phosphate, sodium chloride, potassium chloride, sucrose or trehalose, and optionally a copolymer such as Poloxamer 188 (e.g. Pluronic F-68).

In certain embodiments, the formulation may comprise potassium phosphate, sodium chloride, potassium chloride, Histidine, a sugar, and optionally a copolymer such as Poloxamer 188 (e.g. Pluronic F-68).

In certain embodiments, the formulation may comprise sodium chloride, potassium chloride, sucrose, Tris, and optionally a copolymer such as Poloxamer 188 (e.g. Pluronic F-68).

In certain embodiments, the formulation may comprise sodium chloride, potassium chloride, sucrose, Tris, hydrochloric acid, and optionally a copolymer such as Poloxamer 188 (e.g. Pluronic F-68).

In certain embodiments, the formulation may comprise sodium chloride, sucrose, Tris, and optionally a copolymer such as Poloxamer 188 (e.g. Pluronic F-68),

In certain embodiments, the formulation may comprise sodium chloride, sucrose, Tris, magnesium chloride, and optionally a copolymer such as Poloxamer 188 (e.g. Pluronic F-68).

In certain embodiments, the formulation may comprise sodium chloride, sucrose. Tris, arginine and optionally a copolymer such as Poloxamer 188 (e.g. Pluronic F-68).

In certain embodiments, the formulation may comprise sodium chloride, sorbitol, Iris, and optionally a copolymer such as Poloxamer 188 (e.g. Pluronic F-68),

In certain embodiments, the formulation may comprise sodium chloride, sucrose, Histidine and optionally a copolymer such as Poloxamer 188 (e.g. Pluronic F-68).

In certain embodiments, the formulation may comprise sodium chloride, sucrose, and optionally a copolymer such as Poloxamer 188 (e.g, Pluronic F-68). In certain embodiments, the formulation may comprise 105 mM sodium chloride, 5% (w/v) sucrose, and optionally a copolymer such as Poloxamer 188. In certain embodiments, the formulation may comprise 95 mM sodium chloride, 5% (w/v) sucrose, and optionally a copolymer such as Poloxamer 188. In certain embodiments, the formulation may comprise 220 mM sodium chloride, 5% (w/v) sucrose, and optionally a copolymer such as Poloxamer 188.

In certain embodiments, the formulation may comprise potassium phosphate, sucrose, tris and optionally a copolymer such as Poloxamer 188 (e.g. Pluronic F-68).

In certain embodiments, the formulation may comprise potassium chloride, sucrose, tris and optionally a copolymer such as Poloxamer 188 (e.g. Pluronic F-68).

In certain embodiments, the formulation may comprise sodium chloride, Tris, and optionally a copolymer such as Poloxamer 188 (e.g, Pluronic F-68). In certain embodiments, the formulation may comprise 100 mM sodium chloride, 20 mM Tris, and optionally a copolymer such as Poloxamer 188 (mixture pH of 8.0). In certain embodiments, the formulation may comprise 220 mM. sodium chloride, 20 mM Tris, and optionally a copolymer such as Poloxamer 188 (mixture pH of 7.0-8.0). In certain embodiments, the formulation may comprise 290 mM sodium chloride, 20 mM Tris, and optionally a copolymer such as Poloxamer 188 (mixture pH of 8.0). In certain embodiments, the formulation may comprise 305 mM sodium chloride, 20 mM Tris, and optionally a copolymer such as Poloxamer 188 (mixture pH of 8.0). In certain embodiments, the formulation may comprise 2 M sodium chloride, 20 mM Tris, and optionally a copolymer such as Poloxamer 188 (mixture pH of 8.0). In certain embodiments, the formulation may comprise 170 mM sodium chloride, 40 mM Tris, and optionally a copolymer such as Poloxamer 188 (mixture OH of 8.5). In certain embodiments, the formulation may comprise 2 M sodium chloride, 1 M Tris, and optionally a copolymer such as Poloxamer 188 (mixture pH of 7.5).

In certain embodiments, the formulation may comprise sodium chloride, Tris-Bis Propane, and optionally a copolymer such as Poloxamer 188 (e.g. Pluronic F-68). In certain embodiments, the formulation may comprise 200 mM sodium chloride, 50 mM Tris-Bis Propane, and optionally a copolymer such as Poloxamer 188 (mixture pH of 9.0).

In certain embodiments, the formulation may comprise sodium phosphate, sodium chloride and optionally a copolymer such as Poloxamer 188. In certain embodiments, the formulation may comprise 10 mM sodium phosphate, 180 mM sodium chloride and optionally a copolymer such as Poloxamer 188 (mixture pH of 7.3). In certain embodiments, the formulation may include 10 mM sodium phosphate, 180 mM sodium chloride and 0.001% w/v Poloxamer 188 (mixture pH of 7.3). In certain embodiments, the formulation may comprise 20 mM sodium phosphate, 350 mM sodium chloride and optionally a copolymer such as Poloxamer 188 (mixture pH of 7.4). In certain embodiments, the formulation may comprise 50 mM sodium phosphate, 350 mM sodium chloride and optionally a copolymer such as Poloxamer 188 (mixture pH of 7.4).

In certain embodiments, the formulation may comprise sodium phosphate, potassium phosphate, potassium chloride, sodium chloride, and optionally a copolymer such as Poloxamer 188. In certain embodiments, the formulation may comprise 10 mM sodium phosphate, 2 mM Potassium Phosphate, 2.7 mM. Potassium Chloride, 192 mM Sodium Chloride, and optionally a copolymer such as Poloxamer 188 (mixture pH of 7.5).

In certain embodiments, the formulation may comprise sodium citrate, sodium chloride and optionally a copolymer such as Poloxamer 188. In certain embodiments, the formulation may comprise 20 mM sodium citrate, 1 M sodium chloride and optionally a copolymer such as Poloxamer 188 (mixture pH of 6.0). In certain embodiments, the formulation may comprise 10 mM sodium citrate, 350 mM sodium chloride and optionally a copolymer such as Poloxamer 188 (mixture pH of 6.0). In certain embodiments, the formulation may comprise 20 mM sodium citrate, 350 mM. sodium chloride and optionally a copolymer such as Poloxamer 188 (mixture pH of 3.0).

In certain embodiments, the formulation may comprise PBS. In certain embodiments, the formulation may comprise PBS and a sugar and/or a sugar substitute. The formulation may comprise 3-5% w/v) of the sugar and/or sugar substitute to increase stability of the formulation. As a non-limiting example, the formulation is PBS and 3% (w/v) sucrose (VYFORM30). As another non-limiting example, the formulation is PBS and 5% (w/v) sucrose (VYFORM31). As another non-limiting example, the formulation is PBS and 7% (w/v) sucrose, In certain embodiments, the AAV particles of the disclosure may be formulated in PBS, in combination with an ethylene oxide/propylene oxide copolymer (also known as pluronic or poloxamer).

In certain embodiments, the AAV particles of the disclosure may be formulated in PBS with 3% (w/v) sucrose and 0.001%-0.1% (w/v) of Poloxamer 188 (e.g. Pluronic F-68).

In certain embodiments, the AAV particles of the disclosure may be formulated in PBS with 5% (w/v) sucrose and 0.001%-0.1% (w/v) of Poloxamer 188 (e.g. Pluronic F-68).

In certain embodiments, the AAV particles of the disclosure may be formulated in PBS with 0.001%-0.1% (w/v) of Poloxamer 188 (e.g. Pluronic F-68) at a pH of about 7.0.

In certain embodiments, the AAV particles of the disclosure may be formulated in PBS with 0.001%-0.1% (w/v) of Poloxamer 188 (e.g. Pluronic F-68) at a pH of about 7.3.

In certain embodiments, the AAV particles of the disclosure may be formulated in PBS with 0.001%-0.1% (w/v) of Poloxamer 188 (e.g. Pluronic F-68) at a pH of about 7.4.

In certain embodiments, the AAV particles of the disclosure may be formulated in a solution comprising sodium chloride, sodium phosphate and an ethylene oxide/propylene oxide copolymer.

In certain embodiments, the AAV particles of the disclosure may be formulated in a solution comprising 95 mM sodium chloride, 5 mM sodium phosphate dibasic, 5 mM sodium phosphate monobasic, 1.5 mM potassium phosphate, 7% w/v sucrose, and 0.001% poloxamer 188 (e.g. Pluronic F-68).

In certain embodiments, the AAV particles of the disclosure may be formulated in a solution comprising about 180 mM sodium chloride, about 10 mM sodium phosphate and about 0.001% poloxamer 188, at a pH of about 7.3. The concentration of sodium chloride in the final solution may be 150 mM-200 mM. As non-limiting examples, the concentration of sodium chloride in the final solution may be 150 mM, 160 mM, 170 mM, 180 mM, 190 mM or 200 mM. The concentration of sodium phosphate in the final solution may be 1 mM-50 mM. As non-limiting examples, the concentration of sodium phosphate in the final solution may be 1 mM, 2 mM 3 mM, 4 mM, 5 mM, 6 mM 7 mM, 8 mM, 9 mM, 10 mM, 15 mM, 20 mM 25 mM, 30 mM, 40 mM, or 50 mM. The concentration of poloxamer 188 (Pluronic F-68) may be 0.0001%-1% (w/v). As non-limiting examples, the concentration of poloxamer 188 (Pluronic F-68) may be 0.0001%, 0.0005%, 0.001%, 0.005%, 0.01%, 0.05%, 0.1%, 0.5%, or 1% (w/v). The final solution may have a pH of 6.8-7.7. Non-limiting examples for the pH of the final solution comprise a pH of 6.8, 6.9, 7.0, 7.1, 7.2, 7.3, 7.4, 7.5, 7.6, or 7.7,

In certain embodiments, the AAV particles of the disclosure may be formulated in a solution comprising about 1.05% (w/v) sodium chloride, about 0.212% (w/v) sodium phosphate dibasic, heptahydrate, about 0.025% (w/v) sodium phosphate monobasic, monohydrate, and 0.001% (w/v) poloxamer 188, at a pH of about 7.4. As a non-limiting example, the concentration of AAV particle in this formulated solution may be about 0.001% (w/v). The concentration of sodium chloride in the final solution may be 0.1-2.0% (w/v), with non-limiting examples of 0.1%, 0.25%, 0.5%, 0.75%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.25%, 1.5%, 1.75%, or 2% (w/v). The concentration of sodium phosphate dibasic in the final solution may be 0.100-0.300% (w/v) with non-limiting examples comprising 0.100%, 0.125%, 0.150%, 0.175%, 0.200%, 0.210%, 0.211%, 0.212%, 0.213%, 0.214%, 0.215%, 0.225%, 0.250%, 0.275%, 0.300% (w/v). The concentration of sodium phosphate monobasic in the final solution may be 0.010-0.050% (w/v), with non-limiting examples of 0.010%, 0.015%, 0.020%, 0.021%, 0.022%, 0.023%, 0.024%, 0.025%, 0.026%, 0.027%, 0.028%, 0.029%, 0.030%, 0.035%, 0.040%, 0.045%, or 0.050% (w/v). The concentration of poloxamer 188 (Pluronic F-68) may be 0.0001%-1% (w/v). As non-limiting examples, the concentration of poloxamer 188 (Pluronic F-68)) may be 0.0001%, 0.0005%, 0.001%, 0.005%, 0.01%, 0.05%, 0,1%, 0.5%, or 1% (w/v). The final solution may have a pH of 6.8-7.7. Non-limiting examples for the pH of the final solution comprise a pH of 6.8, 6.9, 7.0, 7.1, 7.2, 7.3, 7.4, 7.5, 7.6, or 7.7.

In certain embodiments, the formulation comprises components with the following CAS (Chemical Abstracts Services) Registry Numbers, 7647-14-15 (sodium chloride), 7782-85-6 (sodium phosphate dibasic, heptahydrate), 10049-21-5 (sodium phosphate monobasic, monohydrate), and 9003-11-6 (poloxamer 188).

### Injectable Formulations

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose, any bland fixed oil can he employed comprising synthetic mono- or diglycerides. Fatty acids such as oleic acid can be used in the preparation of injectables.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of active ingredients, it is often desirable to slow the absorption of active ingredients from subcutaneous or intramuscular injections. This may be accomplished by the use of liquid suspensions of crystalline or amorphous material with poor water solubility. The rate of absorption of active ingredients depends upon the rate of dissolution which, in turn, may depend upon crystal size and crystalline form. Alternatively, delayed absorption of a parenterally administered drug form is accomplished by dissolving or suspending the drug in an oil vehicle, Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers comprise poly(orthoesters) and poly(anhydrides). Depot injectable formulations are prepared by entrapping the drug in liposomes or microemulsions which are compatible with body tissues.

### Depot Formulations

In certain embodiments of the present disclosure, AAV particle formulations of the present disclosure are formulated in depots for extended release. Generally, specific organs or tissues (“target tissues”) are targeted for administration.

In certain embodiments of the disclosure, pharmaceutical compositions, AAV particle formulations of the present disclosure are spatially retained within or proximal to target tissues. Provided are methods of providing pharmaceutical compositions, AAV particle formulations, to target tissues of mammalian subjects by contacting target tissues (which comprise one or more target cells) with pharmaceutical compositions, AAV particle formulations, under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions, AAV particle formulations, that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99% or greater than 99.99% of pharmaceutical compositions, AAV particle formulations, administered to subjects are present intracellularly at a period of time following administration.

Certain aspects of the disclosure are directed to methods of providing pharmaceutical compositions, AAV particle formulations of the present disclosure to a target tissues of mammalian subjects, by contacting target tissues (comprising one or more target cells) with pharmaceutical compositions, AAV particle formulations under conditions such that they are substantially retained in such target tissues. Pharmaceutical compositions, AAV particles comprise enough active ingredient such that the effect of interest is produced in at least one target cell.

### IV. Administration

### Administration

The present disclosure provides methods of administering AAV particles in accordance with the present disclosure to a subject in need thereof In certain embodiments, the formulated AAV particle may be administered to a subject for the treatment of various diseases, disorders and/or conditions. In certain embodiments, the AAV particle may be administered to a subject in a therapeutically effective amount to reduce the symptoms of disease of a subject (e.g., determined using a known evaluation method).

The present disclosure provides a method of delivering to a cell or tissue the AAV particles of the present disclosure, comprising contacting the cell or tissue with said AAV particle or contacting the cell or tissue with a formulation comprising said AAV particle, or contacting the cell or tissue with any of the described compositions, comprising pharmaceutical compositions. The method of delivering the AAV particle to a cell or tissue can be accomplished in vitro, ex vivo, or in vivo.

The present disclosure provides a method of delivering to a subject, comprising a mammalian subject, an AAV particle of the present disclosure comprising administering to the subject said AAV particle, or administering to the subject a formulation comprising said AAV particle, or administering to the subject any of the described compositions, comprising pharmaceutical compositions.

In certain embodiments, the AAV particles and formulations of the present disclosure may be administered by any delivery route which results in a therapeutically effective outcome. These comprise, but are not limited to, enteral (into the intestine), gastroenteral, epidural (into the dura mater), oral (by way of the mouth), transdermal, intracerebral (into the cerebrum), intracerebroventricular (into the cerebral ventricles), sub-pial (between pia and CNS parenchyma), intracarotid arterial (into the intracarotid artery), epicutaneous (application onto the skin), intradermal, (into the skin itself), subcutaneous (under the skin), nasal administration (through the nose), intravenous (into a vein), intravenous bolus, intravenous drip, intra-arterial (into an artery), systemic, intramuscular (into a muscle), intracardiac (into the heart), intraosseous infusion (into the bone marrow), intrathecal (into the spinal canal), intraparenchymal (the body of a tissue or organ, e.g., brain, spinal cord, etc.), intraperitoneal, (infusion, or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), or in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intraabdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracorona, (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intmepideimal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralytnphatic (within the lymph), intramedullary (within the marrow cavity of a bone), intrameningeal (within the meninges), intramyocardial (within the myocardium), intraocular (within the eve), intraovarian (within the ovary), intrapericardial (within the pericardium), intrapleural (within the pleura), intraprostatic (within the prostate gland), intrapulmonary (within the lungs or its bronchi). intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

In certain embodiments, compositions may be administered in a way which allows them to cross the blood-brain barrier, vascular barrier, or other epithelial barrier. The AAV particles of the present disclosure may be administered in any suitable form., either as a liquid solution or suspension, as a solid form suitable for liquid solution or suspension in a liquid solution. The AAV particles may be formulated with any appropriate and pharmaceutically acceptable excipient. In certain embodiments, a composition of the present disclosure is administered to a subject in need intravenously, intramuscularly, subcutaneously, intraperitoneally, intraparenchymally, intrathecally and/or intraventricularly, allowing the formulated AAV particles to pass through one or bath the blood-brain barrier and the blood spinal cord barrier.

The present disclosure provides methods of administering AAV particles in accordance with the present disclosure to the CNS of a subject in need thereof. In certain embodiments, the AAV particle may be administered to the CNS of a subject in a therapeutically effective amount to reduce the symptoms of a neurological disease of a subject, as determined using a known evaluation method.

In certain embodiments, the composition comprising the formulated AAV particles of the present disclosure is administered to the central nervous system of the subject via systemic administration. In certain embodiments, the systemic administration is intravenous injection.

In certain embodiments, the composition comprising the formulated AAV particles of the present disclosure is administered to the central nervous system of the subject via intraparenchymal injection. Non-limiting examples of intraparenchymal injections comprise intrathalamic, intrastriatal (e.g. intraputamenal), intrahippocampal or targeting the entorhinal cortex.

In certain embodiments, the administration into the CNS is via multiple administrations, e.g., more than one, route and/or location. Such administration may be sequential or simultaneous. In certain embodiments, the administration is into the central nervous system of the subject via more than one route, e.g., intraparenchymal injection and intrathecal injection.

In certain embodiments, the administration is via multiple administrations, e.g., more than one, route and/or location. Such administration may be sequential or simultaneous.

In certain embodiments, the formulated AAV particles of the present disclosure may be delivered into specific types of targeted cells, comprising, but not limited to, hippocampal, cortical, motor or entorhinal neurons; glial cells comprising oligodendrocytes. astrocytes and microglia; and/or other cells surrounding neurons such as T cells.

In certain embodiments, the formulated AAV particles or pharmaceutical compositions of the present disclosure may be administered or delivered using the methods for treatment of disease described in U.S. Pat. No. 8,999,948, or International Publication No. WO2014178863, the contents of which are herein incorporated by reference in their entirety.

In certain embodiments, the formulated AAV particles or pharmaceutical compositions of the present disclosure may be administered or delivered using the methods for delivering gene therapy in Alzheimer's Disease or other neurodegenerative conditions as described in US Application No. 20150126590, the contents of which are herein incorporated by reference in their entirety.

In certain embodiments, the formulated AAV particles or pharmaceutical compositions of the present disclosure may be administered or delivered using the methods for delivery of a CNS gene therapy as described in U.S. Pat. Nos. 6,436,708, and 8,946,152, and International Publication No. WO2015168666, the contents of which are herein incorporated by reference in their entirety.

In certain embodiments, the formulated AAV particle or pharmaceutical compositions of the present disclosure may be administered or delivered using the methods for delivering proteins using AAV panicles described in European Patent Application No. EP2678433, the contents of which are herein incorporated by reference in their entirety.

In certain embodiments, the formulated AAV particle or pharmaceutical compositions of the present disclosure may be administered or delivered using the methods for delivering DNA to the bloodstream described in U.S. Pat. No. 6,211,163, the contents of which are herein incorporated by reference in their entirety.

In certain embodiments, the formulated AAV particle or pharmaceutical compositions of the present disclosure may be administered or delivered using the methods for delivering a payload to the central nervous system described in U.S. Pat. No. 7,588,757, the contents of which are herein incorporated by reference in their entirety.

In certain embodiments, the thrmulated AAV particle or pharmaceutical compositions of the present disclosure may be administered or delivered using the methods for delivering; a payload described in U.S. Pat. No. 8,283,151, the contents of which are herein incorporated by reference in their entirety.

In certain embodiments, the formulated AAV particle or pharmaceutical compositions of the present disclosure may be administered or delivered using the methods for delivering a payload using a glutamic acid decarboxylase (GAD) delivery vector described in International Patent Publication No. WO2001089583, the contents of which are herein incorporated by reference in their entirety.

In certain embodiments, the formulated AAV particle or pharmaceutical compositions of the present disclosure may be administered or delivered using the methods for delivering a payload to neural cells described in International Patent Publication No. WO2012057363, the contents of which are herein incorporated by reference in their entirety.

In certain embodiments, the formulated AAV particle or pharmaceutical compositions of the present disclosure may be administered or delivered using the methods for delivering a payload to neural cells described in International Patent Application No. PCT/US2018/042391, the contents of which are herein incorporated by reference in their entirety.

In certain embodiments, the formulated AAV particle or pharmaceutical compositions of the present disclosure may be administered or delivered using the methods for delivering a payload by intramuscular delivery route described in U.S. Pat. No. 6,506,379; the content of which is incorporated herein by reference in its entirety. Non-limiting examples of intramuscular administration comprise an intravenous injection or a subcutaneous injection.

In certain embodiments, the AAV particle formulations of the present disclosure may be delivered by oral administration. Non-limiting examples of oral administration comprise a digestive tract administration and a buccal administration.

In certain embodiments, the AAV particle formulations of the present disclosure may be delivered by intraocular delivery route. A non-limiting example of intraocular administration comprise an intravitreal injection.

In certain embodiments, the AAV particles that may be administered to a subject by peripheral injections. Non-limiting examples of peripheral injections comprise intraperitoneal, intramuscular, intravenous, conjunctival or joint injection. It was disclosed in the art that the peripheral administration of AAV particles can be transported to the central nervous system, for example, to the motor neurons (e.g., U.S. Patent Publication Nos. 20100240739; and 20100130594; the content of each of which is incorporated herein by reference in their entirety).

In certain embodiments, the AAV particle formulations may be delivered by injection into the CSF pathway. Non-limiting examples of delivery to the CSF pathway comprise intrathecal and intracerebroventricular administration.

In certain embodiments, the AAV particle formulations may be delivered by systemic delivery. As a non-limiting example, the systemic delivery may be by intravascular administration.

In certain embodiments, the AAV particle formulations of the present disclosure may be administered to a subject by intracranial delivery (See. e.g., U.S. Pat. No. 8,119,611; the content of which is incorporated herein by reference in its entirety).

In certain embodiments, the AAV particle formulations of the present disclosure may be administered by injection. As a non-limiting example, the AAV particles of the present disclosure may be administered to a subject by injection.

In certain embodiments, the AAV particle formulations of the present disclosure may be administered by muscular injection. As a non-limiting example, the AAV particles of the present disclosure may be administered to a subject by muscular administration.

In certain embodiments, the AAV particle formulations of the present disclosure may be administered by intramuscular administration. As a non-limiting example, the AAV particles of the present disclosure may be administered to a subject by intramuscular administration.

In certain embodiments, the AAV particles of the present disclosure are administered to a subject and transduce muscle of a subject.

In certain embodiments, the AAV particle formulations of the present disclosure may be administered via intraparenchymal injection. As a non-limiting example, the AAV particles of the present disclosure may be administered to a subject by intraparenchymal administration.

In certain embodiments, the AAV particle formulations of the present disclosure may be administered by intravenous administration. In certain embodiments, the AAV particle formulations of the present disclosure may be administered via a single dose intravenous delivery. As a non-limiting example, the single dose intravenous delivery may be a one-time treatment. In the context of gene therapy, the single dose intravenous delivery can produce durable relief for subjects. The relief may last for minutes such as, but not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 33 34, 35, 36, 37, 38, 39, 40, 41, 42, 43, 44, 45, 46, 47, 48, 49, 50, 51, 52, 53, 54, 55, 56, 57, 58, 59 minutes or more than 59 minutes; hours such as, but not limited to, 1, 2, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more than 48 hours; days such as, but not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or more than 31 days; weeks such as, but not limited to, 1, 2, 3, 4, 5, 6, 7, 8, 9, 10, 11, 12, 13, 14, 15, 16, or more than 16 weeks; months such as, but not limited to, 1, 2, 3, 4, 5, 6, 7, 8, 9, 10, 11, 12, 13, 14, 15, 16, 17, 18, 19, 20, 21, 22, 23, 24, or more than 24 months; years such as, but not limited to, 1, 2, 3, 4, 5, 6, 7, 8, 9, 10, 11, 12, 13, 14, 15, or more than 15 years.

In certain embodiments, the AAV particle formulations of the present disclosure may be administered via intravenous delivery to the DRG nociceptive neurons. In certain embodiments, the AAV particle formulations of the present disclosure may be administered via a single dose intravenous delivery to the DRG nociceptive neurons. As a non-limiting example, the single dose intravenous delivery may he a one-time treatment. The relief may last for minutes such as, but not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 minutes or more than 59 minutes; hours such as, but not limited to, 1, 2, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more than 48 hours; days such as, but not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or more than 31 days; weeks such as, but not limited to, 1, 2, 3, 4, 5, 6, 7, 8, 9, 10, 11, 12, 13, 14, 15, 16, or more than 16 weeks; months such as, but not limited to, 1, 2, 3, 4, 5, 6, 7, 8, 9, 10, 11, 12, 13, 14, 15, 16, 17, 18, 19, 20, 21, 22, 23, 24, or more than 24 months; years such as, but not limited to, 1, 2, 3, 4, 5, 6, 7, 8, 9, 10, 11, 12, 13, 14, 15, or more than 15 years.

In certain embodiments, the AAV particle formulations of the present disclosure may be administered intrathecally, such as by intrathecal injection. In certain embodiments, the AAV particle formulations may be administered to the cisterna magna in a therapeutically effective amount to transduce spinal cord motor neurons and/or astrocytes. As a non-limiting example, the AAV particle formulations may be administered intrathecally. In certain embodiments, the AAV particle formulations of the present disclosure may be administered via a single dose intrathecal injection. As a non-limiting example, the single dose intrathecal injection may he a one-time treatment and the single dose intrathecal injection can produce durable relief for subjects. The relief may last for minutes such as, but not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 minutes or more than 59 minutes; hours such as, but not limited to, 1, 2, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more than 48 hours; days such as, but not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or more than 31 days; weeks such as, but not limited to, 1, 2, 3, 4, 5, 6, 7, 8, 9, 10, 11, 12, 13, 14, 15, 16, or more than 16 weeks; months such as, but not limited to, 1, 2, 3, 4, 5, 6, 7, 8, 9, 10, 11, 12, 13, 14, 15, 16, 17, 18, 19, 20, 21, 22, 23, 24, or more than 24 months; years such as, but not limited to, 1, 2, 3, 4, 5, 6, 7, 8, 9, 10, 11, 12, 13, 14, 15, or more than 15 years.

In certain embodiments, the AAV particle formulations of the present disclosure may be administered via intrathecal injection to the DRG nociceptive neurons. In certain embodiments, the AAV particle formulations of the present disclosure may be administered via a single dose intrathecal injection to the DRG nociceptive neurons. As a non-limiting example, the single dose intrathecal injection may be a one-time treatment. In the context of gene therapy, the single dose intrathecal injection can produce durable relief for subjects. The relief may last for minutes such as, but not limited to, 1, 2, 3, 4, 5 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42. 43, 44, 45, 46, 47, 48, 49, 50, 51, 52, 53, 54, 55, 56, 57, 58, 59 minutes or more than 59 minutes; hours such as, but not limited to, 1, 2, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 37, 38, 39, 40, 41, 42, 43, 44, 45, 46, 47, 48, or more than 48 hours; days such as, but not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or more than 31 days; weeks such as, but not limited to, 1, 2, 3, 4, 5, 6, 7, 8, 9, 10, 11, 12, 13, 14, 15, 16, or more than 16 weeks; months such as, but not limited to, 1, 2, 3, 4, 5, 6, 7, 8, 9, 10, 11, 12, 13, 14, 15, 16, 17, 18, 19, 20, 21, 22, 23, 24, or more than 24 months; years such as, but not limited to, 1, 2, 3, 4, 5, 6, 7, 8, 9, 10, 11, 12, 13, 14, 15, or more than 15 years.

In certain embodiments, the formulated AAV particle described herein is administered via intrathecal (IT) infusion at Cl. The infusion may be for 1, 2, 3, 4, 6, 7, 8, 9, 10, 11, 12, 13, 14, 15 or more than 15 hours.

In certain embodiments, the AAV particle formulations of the present disclosure may be administered by intraparenchymal injection. In certain embodiments, the formulated AAV particle may be administered via intraparenchymal injection to the cisterna magna in a therapeutically effective amount to transduce spinal cord motor neurons and/or astrocytes.

In certain embodiments, the AAV particle formulations of the present disclosure may be administered by intraparenchymal injection and intrathecal injection.

In certain embodiments, the AAV particle formulations of the present disclosure may be administered by subcutaneous injection.

In certain embodiments, the AAV particle formulations may be delivered by direct injection into the brain. In certain embodiments, the AAV particle formulations of the present disclosure may be administered via intrastriatal injection, comprising injection into one or more putamen. In certain embodiments, the AAV particle formulations of the present disclosure may be administered via intrastriatal injection and another route of administration described herein.

In certain embodiments, the AAV particle formulations may be delivered by more than one route of administration. As non-limiting examples of combination administrations, AAV particles may be delivered by intrathecal and intracerebroventricular, or by intravenous and intraparenchymal administration.

In certain embodiments, the formulated AAV particle may be administered to the CNS in a therapeutically effective amount to improve function and/or survival for a subject. As a non-limiting example, the vector may be administered by direct infusion into the striatum,

In certain embodiments, the formulated AAV particle may be administered in a “therapeutically effective” amount, i.e., an amount that is sufficient to alleviate and/or prevent at least one symptom associated with the disease or provide improvement in the condition of the subject.

In certain embodiments, the catheter may be located at more than one site in the spine for multi-site delivery. The formulated AAV particle may be delivered in a continuous and/or bolus infusion. Each site of delivery may be a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In certain embodiments, a subject may be analyzed for spinal anatomy and pathology prior to delivery of the AAV particle described herein. As a non-limiting example, a subject with scoliosis may have a different dosing regimen and/or catheter location compared to a subject without scoliosis.

In certain embodiments, the orientation of the spine of the subject during delivery of the formulated AAV particle may be vertical to the ground.

In another embodiment, the orientation of the spine of the subject during delivery of the formulated AAV particle may be horizontal to the ground.

In certain embodiments, the spine of the subject may be at an angle as compared to the ground during the delivery of the AAV particle formulations. The angle of the spine of the subject as compared to the ground may be at least 10, 20, 30, 40, 50, 60, 70, 80, 90, 100, 110, 120, 130, 140, 150 or 180 degrees.

In certain embodiments, the delivery method and duration are chosen to provide broad transduction in the spinal cord. As a non-limiting example, intrathecal delivery is used to provide broad transduction along the rostral-caudal length of the spinal cord. As another non-limiting example, multi-site infusions provide a more uniform transduction along the rostral-caudal length of the spinal cord. As yet another non-limiting example, prolonged infusions provide a more uniform transduction along the rostral-caudal length of the spinal cord.

### Dosage and Regimen

The pharmaceutical, diagnostic, or prophylactic AAV particles, formulations and compositions of the present disclosure may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal. Compositions in accordance with the present disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate diagnostic dose level for any particular individual will depend upon a variety of factors comprising the disorder being treated and the severity of the disorder; the activity of the specific payload employed; the specific composition employed; the age, body weight, general health, sex and diet of the patient; the time of administration, route of administration, and rate of excretion of the specific AAV particle employed; the duration of the treatment; drugs used in combination or coincidental with the specific AAV particle employed; and like factors well known in the medical arts. In certain embodiments, delivery of the formulated AAV particles of the present disclosure results in minimal serious adverse events (SAEs) as a result of the delivery of the formulated AAV particles.

Traditionally, in order to achieve a sufficiently high quantity of a standard therapeutic (ensuring long lasting therapeutic effects), many therapies are delivered by repeated or extended administration, e.g. by multiple injections or long infusions. However, these extended dosing regimens are not well suited for certain AAV gene therapy modalities, such as those driven by single dose paradigms. Extended AAV dosing regimens can be particularly problematic in the treatment of CNS diseases due to limited volumetric space for formulation fluids, strinaent limitations on excipient quantity and potency, impact of ionic particles on CNS micro-environment, and surgical and clinical complications related to extended CNS treatments. Novel formulations of gene therapy modalities are necessary to achieve sufficiently high concentrations of therapeutic agents within the formulation.

In certain embodiments, formulated AAV particle pharmaceutical compositions in accordance with the present disclosure may be administered at dosage levels sufficient to deliver from about 0.0001 mg/kg to about 100 mg/kg, from about 0.001 mg/kg to about 0.05 ma/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect. It will be understood that the above dosing concentrations may be converted to vg or viral genomes per kg or into total viral genomes administered by one of skill in the art.

In certain embodiments, formulated AAV particle pharmaceutical compositions in accordance with the present disclosure may be administered at about 10 to about 600 μl/site, 50 to about 500 μl/site, 100 to about 400 μl/site, 120 to about 300 μl/site, 140 to about 200 μl/site, about 160 μl/site. As non-limiting examples, AAV particles may be administered at 50 μl/site and/or 150 μl/site.

In certain embodiments, delivery of the compositions in accordance with the present disclosure to cells comprises a rate of delivery defined by [VG/hour=ml/hour*VG/mL] wherein VG is viral genomes, VG/mL is composition concentration, and mL/hour is rate of prolonged delivery.

In certain embodiments, delivery of compositions in accordance with the present disclosure to cells may comprise a total concentration per subject between about 1×10⁶ VG and about 1×10¹⁶ VG. In certain embodiments, delivery may comprise a composition concentration of about 1×10⁶, 2×10⁶, 3×10⁶, 4×10⁶, 5×10⁵, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁹, 3×10¹⁰, 4×10¹⁰, 5×1¹⁰, 6×10¹⁰, 7×10¹⁰, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×10¹¹, 8×10¹¹, 9×10¹¹, 1×10¹², 1.1×10¹², 1,2×10¹², 1.3×10¹², 1.4×10¹², 1.5×10¹², 1.6×10¹², 1.7×10¹², 1.8×10¹², 1.9×10¹², 2×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10¹², 8.2×10¹², 8.3×10¹², 8.4×10¹², 8.5×10¹², 8.6×10¹², 8.7×10¹², 8.8×10¹², 9×10¹², 9×10¹², 1×10¹³, 2×10¹³, 3×10¹³, 4×10¹², 5×10¹³, 6×10¹³, 6.7×10¹³, 7×10¹³, 8×10¹³ 9×10¹³, 1×10¹⁴, 2×10¹⁴, 3×10¹⁴, 4×10¹⁴, 5×10¹⁴, 6×10¹⁴, 7×10¹⁴, 8×10¹¹, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/subject or VG/dose.

In certain embodiments, delivery of compositions in accordance with the present disclosure to cells may comprise a total concentration per subject between about 1×10⁶ VG/kg and about 1×10¹⁶ VG/kg. In certain embodiments, delivery may comprise a composition concentration of about 1×10⁶, 2×10⁶, 3×10⁶, 4×10⁶, 5×10⁶ 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁵, 9×10⁸, 1×10⁹, 2×10⁹, 3×10⁹, 4×10⁹, 5×10⁹, 6×10⁹, 7×10⁹, 8×10⁹, 9×10⁹, 1×10¹⁶, 2×10¹⁰, 3×10¹⁰, 4×10¹⁹, 5×10¹⁹, 6×1e, 7×10¹⁹, 8×10¹⁰, 9×10¹⁰, 1×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 4×10¹¹, 5×10¹¹, 6×10¹¹, 7×10¹¹, 7.1×10¹¹, 7.2×10¹¹, 7.3×10¹¹, 7.4×10¹¹, 7.5×10¹¹, 7.6×10¹¹, 7.7×10¹¹, 7.8×10¹¹, 7.9×1.0¹¹, 8×10¹¹, 9×10¹¹, 1×10¹², 1.1 ×10¹², 1.2×10¹², 1.3×10¹², 1.4×10¹², 1.5×10¹², 1.6×10¹², 1.7×10¹², 1.8×10¹², 1.9×10¹², 2×10¹², 3×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 8.1×10¹², 8.2×10¹², 8.3×10¹², 8.4×10¹², 8.5×10¹², 8.6×10¹², 8.7×10¹², 8.8 ×10¹², 8.9×10¹², 9×10¹², 1×10¹³, 2×10¹³, 3×10¹³, 4×10¹², 5×10¹², 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/ka.

In certain embodiments, delivery of formulated AAV particles to cells of the central nervous system (e.g., parenchyma) may comprise a total dose between about 1×10⁶ VG and about 1×10¹⁶ VG. In certain embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁹, 7×10¹⁹, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG. As a non-limiting example, the total dose is 1×10¹³ VG. As another non-limiting example, the total dose is 2.1×10¹² VG.

In certain embodiments, about 10⁵ to 10⁶ viral genome (unit) may be administered per dose.

In certain embodiments, delivery of the compositions in accordance with the present disclosure to cells may comprise a total concentration between about 1×10⁶ VG/mL and about 1×10¹⁶ Vg/mL. In certain embodiments, delivery may comprise a composition concentration of about 1×10⁶, 2×10⁶, 3×10⁶, 4×10⁶, 5×10⁶, 6×10^{,} 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 1.4×10¹², 1.5×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.8×10¹², 2.9×10¹², 3×10¹², 3.1×10¹², 3.2×10¹², 3.3×1.0¹², 3.4×10¹², 3.5×10¹², 3.6×10¹², 3.7×10¹², 3.8×10¹², 3.9×10¹¹, 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10′'', 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1.×10¹⁶ VG/mL. As used herein, “VG/mL” represents vector genomes (VG) per milliliter (mL). VG/mL also may describe genome copy per milliliter or DNase resistant particle per milliliter.

In certain embodiments, delivery of the compositions in accordance with the present disclosure to cells may comprise a total concentration between about 1×10⁶ total capsid/mL and about 1×10¹⁶ total capsid/mL. In certain embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.1×10¹², 1.2×10¹², 1.3×10¹², 4×10¹², 15×10¹², 1.6×10¹², 1.7×10¹², 1.8×10¹², 1.9×10¹², 2×10¹², 2.1×10¹², 2.2×10¹², 2.3×10¹², 2.4×10¹², 2.5×10¹², 2.6×10¹², 2.7×10¹², 2.80×10¹², 2.9×10¹², 3×10¹², 3.1×10¹², 3.2×10¹², 3.3×10¹², 3.4×10¹², 3.5×10¹², 3.6×10¹⁷, 3.7×10¹², 3.8×10¹², 3.9×10¹², 4×10¹², 4.1×10¹², 4.2×10¹², 4.3×10¹², 4.4×10¹², 4.5×10¹², 4.6×10¹², 4.7×10¹², 4.8×10¹², 4.9×10¹², 5×10¹², 6×10¹², 7×10¹², 8×10¹², 9×10¹², 1×10¹³, 2×10¹³, 3×10¹³, 4×10¹³, 5×10¹³, 6×10¹³, 6.7×10¹³, 7×10¹³, 8×10¹³, 9×10¹³, 1×10¹⁴, 2×10¹⁴, 3×10¹⁴, 40×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ total capsid/mL.

In certain embodiments, delivery of formulated AAV particles to cells of the central nervous system (e.g., parenchyma) may comprise a composition concentration between about 1×10⁶ VG/mL and about 1×10¹⁶ VG/mL. In certain embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁵, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10 ¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹⁰, 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 10×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL. In certain embodiments, the delivery comprises a composition concentration of 1×10¹³ VG/mL. In certain embodiments, the delivery comprises a composition concentration of 2.1×10¹² VG/mL,

The desired dosage of the formulated AAV particles of the present disclosure may be delivered only once, three times a day, two times a day, once a day or more than once in a single administration protocol. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event.

The desired dosage of the formulated AAV particles of the present disclosure may be administered as a “pulse dose” or as a “continuous flow”. As used herein, a “pulse dose” is a series of single unit doses of any therapeutic administered with a set frequency over a period of time. As used herein, a “continuous flow” is a dose of therapeutic administered continuously for a period of time in a single route/single point of contact, i.e., continuous administration event. A total daily dose, an amount given or prescribed in 24-hour period, may be administered by any of these methods, or as a combination of these methods, or by any other methods suitable for a pharmaceutical. administration.

In certain embodiments, delivery of the formulated AAV particles of the present disclosure to a subject provides regulating activity of a gene of interest in a subject. The regulating activity may be an increase in the production of a gene of interest in a subject or the decrease of the production of a gene of interest in a subject. The regulating activity can be for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

In certain embodiments, the formulated AAV particle of the present disclosure may be administered to a subject using a single dose, one-time treatment. The dose of the one-time treatment may be administered by any methods known in the art and/or described herein. As used herein, a “one-time treatment” refers to a composition which is only administered one time. if needed, a booster dose may be administered to the subject to ensure the appropriate efficacy is reached. A booster may be administered 1 month, 2 months, 3 months, 4 months, 5 months, 6 months, 7 months, 8 months, 9 months, 10 months. 11 months, 12 months, 1 year, 13 months, 14 months, 15 months, 16 months, 17 months, 18 months, 19 months, 20 months, 21 months, 22 months, 23 months, 24 months, 2 years, 3 years, 4 years, 5 years, 6 years, 7 years, 8 years, 9 years, 10 years, or more than 10 years after the one-time treatment.

In certain embodiments, the AAV particle formulations of the present disclosure may be delivered to a subject via a single route of administration,

In certain embodiments, the AAV particle formulations of the present disclosure may be delivered to a subject via a multi-site route of administration. A subject may be administered at 2, 3, 4, 5 or more than 5 sites.

In certain embodiments, a subject may be administered the AAV particle formulations of the present disclosure using a bolus infusion.

In certain embodiments, a subject may be administered the AAV particle formulations of the present disclosure using sustained delivery over a period of minutes, hours or days. The infusion rate may be changed depending on the subject, distribution, formulation or another delivery parameter.

In certain embodiments, the AAV particle described herein is administered via putamen and caudate infusion. As a non-limiting example, the dual infusion provides a broad striatal distribution as well as a frontal and temporal cortical distribution.

In certain embodiments, the selection of subjects for administration of the AAV particle described herein and/or the effectiveness of the dose, route of administration and/or volume of administration may be evaluated using imaging of the perivascular spaces (PVS) which are also known as Virchow-Robin spaces. PVS surround the arterioles and venules as they perforate brain parenchyma and are filled with cerebrospinal fluid (CSF)linterstitial fluid. PVS are common in the midbrain, basal ganglia, and centnim semiovale. While not wishing to be bound by theory, PVS may play a role in the normal clearance of metabolites and have been associated with worse cognition and several disease states comprising Parkinson's disease. PVS are usually are normal in size but they can increase in size in a number of disease states. Potter et al. (Cerehrovasc Dis. 2015 January; 39(4): 224-231; the contents of which are herein incorporated by reference in its entirety) developed a grading method where they studied a frill range of PVS and rated basal ganglia, centrum semiovale and midbrain PVS. They used the frequency and range of PVS used by Mac and Luilich et al. (J Neurol Neurosurg Psychiatry. 2004 November;75(11):1519-23; the contents of which are herein incorporated by reference in its entirety) and Potter et al. gave 5 ratings to basal ganglia and centrum semiovale PVS: 0 (none), 1 (1-10), 2 (11-20), 3 (21-40) and 4 (>40) and 2 ratings to midbrain PVS: 0 (non-visible) or 1 (visible). The user guide for the rating system by Potter et al. can be found at: www.shirc.ed.ac.uk/documents/epvs-rating-scale-user-guide.pdf

### Combinations

In certain embodiments, the AAV particle formulations of the present disclosure may be used in combination with one or more other therapeutic, prophylactic, research or diagnostic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. in certain embodiments, the present disclosure encompasses the delivery of pharmaceutical, prophylactic, research, or diagnostic compositions in combination with agents that may improve their bioavailability, reduce and/or modify their metabolism, inhibit their excretion, and/or modify their distribution within the body.

Therapeutic agents that may he used in combination with the formulated AAV particles of the present disclosure can be small molecule compounds which are antioxidants, anti-inflammatory agents, anti-apoptosis agents, calcium regulators, antiglutamatergic agents, structural protein inhibitors, compounds involved in muscle function, and compounds involved in metal ion regulation.

In certain embodiments, compounds tested for treating disease which may be used in combination with the formulated. AAV particles described herein comprise, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, such as Methylene blue, phenothiazines, anthraquinones, n-phenylamines or rhodamines, microtubule stabilizers such as NAP, taxol or paclitaxel, kinase or phosphatase inhibitors such as those targeting GSK3β (lithium) or PP2A, immunization with Aβ peptides or dopamine-depleting agents e.g., tetrabenazine for chorea), benzodiazepines (e.g., clonazepam thr myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acetycholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole thr psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., zopiclone and/or zolpidem for altered sleep-wake cycle), anticonvulsants (e.g., sodium valproate and carbamazepine for mania or hypomania) and mood stabilizers (e.g., lithium for mania or hypomania).

In certain embodiments, neurotrophic factors may be used in combination therapy with the formulated AAV particles of the present disclosure. Generally, a neurotrophic factor is defined as a substance that promotes survival, growth, differentiation, proliferation and/or maturation of a neuron, or stimulates increased activity of a neuron. In certain embodiments, the present methods further comprise delivery of one or more trophic factors into the subject in need of treatment. Trophic factors may comprise, but are not limited to, IGF-I, GDNF, BDNF, CTNF, VEGF, Colivelin, Xaliproden, Thyrotrophin-releasing hormone and ADNF, and variants thereof

In one aspect, the formulated AAV particle described herein may be co-administered with AAV particles (whether or not formulated) expressing neurotrophic factors such as AAV-IGF-I (See e.g., Vincent et al., Neuromolecular medicine, 2004, 6, 79-85; the content of which is incorporated herein by reference in its entirety) and AAV-GDNF (See e.g., Wang et al., J. Neurosci., 2002, 22, 6920-6928; the content of which is incorporated herein by reference in its entirety).

### Measurement and Analysis

Expression of payloads or the downregulating effect of such payloads from viral genomes may be determined using various methods known in the art such as, but not limited to immunochemistry (e.g., II-IC), in situ hybridization (ISM, enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, and/or PCR.

### Bioavailability

In certain embodiments, AAV particles formulated into a composition with a delivery agent as described herein can exhibit an increase in bioavailability as compared to a composition lacking a delivery agent as described herein. As used herein, the term “bioavailability” refers to the systemic availability of a given amount of AAV particle or expressed payload administered to a mammal. Bioavailability can be assessed by measuring the area under the curve (AUC) or the maximum serum or plasma concentration (Cₘₐₓ) of the composition following. AUC is a determination of the area under the curve plotting the serum or plasma concentration of a compound (e.g., AAV particles or expressed payloads) along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, the contents of which are herein incorporated by reference in its entirety.

The Cₘₐₓ value is the maximum concentration of the AAV particle or expressed payload achieved in the serum or plasma of a mammal following administration of the AAV particle to the mammal. The Cₘₐₓ value of can be measured using methods known to those of ordinary skill in the art. The phrases “increasing bioavailability” or “improving the pharmacokinetics,” as used herein mean that the systemic availability of a first AAV particle or expressed payload, measured as AUC, Cₘₐₓ, or Cₘᵢₙ in a mammal is greater, when co-administered with a delivery agent as described herein, than when such co-administration does not take place. In certain embodiments, the bioavailability can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Therapeutic Window

As used herein “therapeutic window” refers to the range of plasma concentrations, or the range of levels of therapeutically active substance at the site of action, with a high probability of eliciting a therapeutic effect. In certain embodiments, the therapeutic window of the AAV particle formulations as described herein can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Volume of Distribution

As used herein, he term “volume of distribution”refers to the fluid volume that would be required to contain the total amount of the drug in the body at the same concentration as in the blood or plasma: Vdist equals the amount of drug in the body/concentration of drug in blood or plasma. For example, for a 10 mg dose and a plasma concentration of 10 mg/L, the volume of distribution would be 1 liter. The volume of distribution reflects the extent to which the drug is present in the extravascular tissue. A large volume of distribution reflects the tendency of a compound to bind to the tissue components compared with plasma protein binding. In a clinical setting, Vdist can be used to determine a loading dose to achieve a steady state concentration. In certain embodiments, the volume of distribution of the AAV particle formulations as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Biologic Effect

In certain embodiments, the biological effect of the AAV particle formulations delivered to the animals may be categorized by analyzing the payload expression in the animals. The payload expression may be determined from analyzing a biological sample collected from a mammal administered the AAV particle formulations of the present disclosure. For example, a protein expression of 50-200 pg/ml for the protein encoded by the AAV particles delivered to the mammal may be seen as a therapeutically effective amount of protein in the mammal.

### V. Treatments

### General

The present disclosure provides a method for treating a disease, disorder and/or condition in a mammalian subject, comprising a human subject, comprising administering to the subject any of the viral particles or formulations described herein or administering to the subject any of the described compositions, comprising pharmaceutical compositions or formulations, described herein.

In certain embodiments, administration of the formulated AAV particles to a subject with not change the course of the underlying disease but will ameliorate symptoms in a subject.

In certain embodiments, the viral particles of the present disclosure are administered to a subject prophylactically.

In certain embodiments, the viral particles of the present disclosure are administered to a subject having at least one of the diseases described herein.

In certain embodiments, the viral particles of the present disclosure are administered to a subject to treat a disease or disorder described herein. The subject may have the disease or disorder or may be at-risk to developing the disease or disorder.

The present disclosure provides a method for administering to a subject in need thereof, comprising a human subject, a therapeutically effective amount of the AAV particles of the present disclosure to slow, stop or reverse disease progression. As a non-limiting example, disease progression may be measured by tests or diagnostic tool(s) known to those skilled in the art. As another non-limiting example, disease progression may be measured by change in the pathological features of the brain, CSF or other tissues of the subject.

In certain embodiments, various non-infectious diseases, comprising neurological diseases, may be treated with pharmaceutical compositions of the present disclosure. AAV particles, especially blood brain barrier crossing AAV particles of the present disclosure, are particularly useful in treating various neurological diseases. As a non-limiting example, the neurological disease may be Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malfortrtation, Asperger Syndrome, Ataxia, Ataxia Telangiectasia, Ataxias and Cerebellar or Spinocerebellar Degeneration, Atrial Fibrillation and Stroke, Attention Deficit-Flyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockavne Syndrome Type II, Coffin Lowry Syndrome, Colpocephaly, Coma, Complex Regional Pain Syndrome, Congenital Facial Diplegia, Congenital Myasthertia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Detnentia, Dementia-Multi-infarct, Dementia-Semantic, Dementia-Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myocionica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gersimann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barr- Syndrome, Hallervorden-Spatz Disease, Head Injury, Headache, Hemicrania Continua, Hemifacial Spasm, Hemiplegia Alterans, Hereditary-Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus-Normal Pressure, Hydromyelia, Hypercortisolism, Hypersomnia, Hypertonia, Hypotonia, Hypoxia, Immne-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsboume syndrome, Kleine-Levin Syndrome, Klippel-Feil Syndrome, Klippel-Trenaunay Syndrome (KTS), KInver-Bucy Syndrome, Korsakoffs Amnesic Syndrome, Krabbe Disease, Kugelberg-Welander Disease, Kum, Lambert-Eaton Myasthenic Syndrome, Landau-Kleffner Syndrome, Lateral Femoral Cutaneous Nellie Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease-Neurological Complications, Machado-Joseph Disease, Macrencephaly, Megalencephaly, Melkersson-Rosenthal Syndrome, Meningitis, Meningitis and Encephalitis, Menkes Disease, Meralgia. Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension., Muscular Dystrophy, Myasthenia-Congenital, Myasthenia. Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotortia, Neuronal Ceroid Lipofuscinosis, Neuronal Migration Disorders, Neuropathy- Hereditary, Neurosarcoidosis, Neurosyphilis, Neurotoxicity, Nevus Cavemosus, Niemann-Pick Disease, O'Sullivan-McLeod Syndrome, Occipital Neuralgia, Ohtahara Syndrome, Olivopontocerebellar Atrophy, Opsoci.onus itilyoclonus, Orthostatic Hypotension, Overuse Syndrome. Pain -Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-omberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease-Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgifortn (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

The present disclosure additionally provides a method for treating neurological disorders in a mammalian subject, comprising a human subject, comprising administering to the subject any of the AAV particles or pharmaceutical compositions of the present disclosure, In certain embodiments, the AAV particle is a blood brain barrier crossing particle. In certain embodiments, neurological disorders treated according to the methods described herein comprise, but are not limited to Amyotrophic lateral sclerosis (ALS), Huntington's Disease (HD), Parkinson's Disease (PD), and/or Friedreich's Ataxia (FA).

### Kits and Devices

### Kits

In some embodiments, the disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the AAV particles of the present disclosure may be comprised in a kit. In some embodiments, kits may further comprise reagents and/or instructions for creating and/or synthesizing compounds and/or compositions of the present disclosure. In some embodiments, kits may also comprise one or more buffers. In some embodiments, kits of the disclosure may comprise components for making protein or nucleic acid arrays or libraries and thus, may comprise, for example, solid supports.

In some embodiments, kit components may be packaged either in aqueous media or in lyophilized form. The container means of the kits will generally comprise at least one vial, test tube, flask, bottle, syringe or other container means, into which a component may be placed, and preferably, suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically comprise means for containing compounds and/or compositions of the present disclosure, proteins, nucleic acids, and any other reagent containers in close confinement for commercial sale. Such containers may comprise injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

In some embodiments, kits may comprise instructions for employing kit components as well the use of any other reagent not comprised in the kit. Instructions may comprise variations that may be implemented.

### Devices

In some embodiments, the AAV particles may delivered to a subject using a device to deliver the AAV particles and a head fixation assembly. The head fixation assembly may be, but is not limited to, any of the head fixation assemblies sold by MRI interventions. As a non-limiting example, the head fixation assembly may be any of the assemblies described in U.S. Pat. Nos. 8,099,150, 8,548,569 and 9,031,636 and International Patent Publication Nos. WO201108495 and WO2014014585, the contents of each of which are incorporated by reference in their entireties. A head fixation assembly may be used in combination with an MRI compatible drill such as, but not limited to, the MRI compatible drills described in International Patent Publication No. WO402013181008 and US Patent Publication No. US0520130325012, the contents of which are herein incorporated by reference in its entirety.

In some embodiments, the AAV particles may be delivered using a method, system and/or computer program for positioning apparatus to a target point on a subject to deliver the AAV particles. As a non-limiting example, the method, system andlor computer program may be the methods, systems and/or computer programs described in U.S. Pat. No. 8,340,743, the contents of which are herein incorporated by reference in its entirety. The method may comprise: determining a target point in the body and a reference point, wherein the target point and the reference point define a planned trajectory line (PTL) extending through each; determining a visualization plane, wherein the PTL intersects the visualization plane at a sighting point; mounting the guide device relative to the body to move with respect to the PTL, wherein the guide device does not intersect the visualization plane; determining a point of intersection (GPP) between the guide axis and the visualization plane; and aligning the GPP with the sighting point in the visualization plane.

In some embodiments, the AAV particles may be delivered to a subject using a convention-enhanced delivery device. Non-limiting examples of targeted delivery of drugs using convection are described in US Patent Publication Nos. US20100217228, US20130035574 and US20130035660 and International Patent Publication No. WO2013019830 and WO2008144585, the contents of each of which are herein incorporated by reference in their entireties.

In some embodiments, a subject may be imaged prior to, during and/or after delivery of the AAV particles. The imaging method may be a method known in the art andlor described herein, such as but not limited to, magnetic resonance imaging (MRI). As a non-limiting example, imaging may be used to assess therapeutic effect. As another non-limiting example, imaging may be used for assisted delivery of AAV particles, 11.0591 In some embodiments, the AAV particles may be delivered using an MRI-guided device. Non-limiting examples of MRI-guided devices are described in U.S. Pat. Nos. 9,055,884, 9,042,958, 8,886,288, 8,768,433, 8,396,532, 8,369,930, 8,374,677 and 8,175,677 and US Patent Application No. US20140024927 the contents of each of which are herein incorporated by reference in their entireties. As a non-limiting example, the MM-guided device may be able to provide data in real time such as those described in US Patent Nos. 8,886,288 and 8,768,433, the contents of each of which is herein incorporated by reference in its entirety. As another non-limiting example, the MRI-guided device or system may be used with a targeting cannula such as the systems described in U.S. Pat. Nos. 8,175,677 and 8,374,677, the contents of each of which are herein incorporated by reference in their entireties. As yet another non-limiting example, the MRI-guided device comprises a. trajectory guide frame for guiding an interventional device as described, for example, in U.S. Pat. No. 9,055,884 and US Patent Application No. US20140024927, the contents of each of which are herein incorporated by reference in their entireties.

In some embodiments, the AAV particles may be delivered using an MRI-compatible tip assembly. Non-limiting examples of MRI-compatible tip assemblies are described in US Patent Publication No. US20140275980, the contents of which is herein incorporated by reference in its entirety.

In some embodiments, the AAV particles may be delivered using a cannula which is MM-compatible. Non-limiting examples of MM-compatible cannulas comprise those taught in International Patent Publication No. WO2011130107, the contents of which are herein incorporated by reference in its entirety. In some embodiments, the cannula or a portion thereof or the tubing associated with the cannula is attached, mounted, glued, affixed or otherwise makes reversible contact with the tissue surrounding the surgical site/field. Such contact may be localized andlor stabilized in one position during all or a portion of the procedure.

In some embodiments, the AAV particles may be delivered using a catheter which is MRI-compatible. Non-limiting examples of MRI-compatible catheters comprise those taught in International Patent Publication No. WO2012116265, US Patent Publication No. 8,825,133 and US Patent Publication No. US20140024909, the contents of each of which are herein incorporated by reference in their entireties.

In some embodiments, the AAV particles may be delivered using a device with an elongated tubular body and a diaphragm as described in US Patent Publication Nos. US20140276582 and US20140276614, the contents of each of which are herein incorporated by reference in their entireties.

In some embodiments, the AAV particles may be delivered using an MRI compatible localization and/or guidance system such as, but not limited to, those described in US Patent Publication Nos. U.S. Pat. No. 20150223905 and U.S. Pat. No. 20150230871, the contents of each of which are herein incorporated by reference in their entireties. As a non-limiting example, the MRI compatible localization and/or guidance systems may comprise a mount adapted for fixation to a patient, a targeting cannula with a lumen configured to attach to the mount so as to be able to controllably translate in at least three dimensions, and an elongate probe configured to snugly advance via slide and retract in the targeting cannula lumen, the elongate probe comprising at least one of a stimulation or recording electrode.

In some embodiments, the AAV particles may he delivered to a subject using a trajectory frame as described in US Patent Publication Nos. US20150031982 and US20140066750 and International Patent Publication Nos. WO2015057807 and WO2014039481, the contents of each of which are herein incorporated by reference in their entireties.

In some embodiments, the AAV particles may be delivered to a subject using a gene gun.

### Use of AAV Particles Encoding Protein Payloads

Provided in the present disclosure are methods for introducing into cells the AAV particles manufactured according to the methods and systems of the present disclosure, the methods comprising introducing into said cells any of the vectors in an amount sufficient for an increase in the production of target mRNA and protein to occur. In some aspects, the cells may be muscle cells, stem cells, neurons such as but not limited to, motor, hippocampal, entorhinal, thalamic or cortical neurons, and glial cells such as astrocytes or microglia.

Disclosed in the present disclosure are methods for treating neurological disease associated with insufficient function/presence of a target protein in a subject in need of treatment, The method optionally comprises administering to the subject a therapeutically effective amount of a composition comprising AAV particles of the present disclosure. As a non-limiting example, the AAV particles can increase target gene expression, increase target protein production, and thus reduce one or more symptoms of neurological disease in the subject such that the subject is therapeutically treated.

In certain embodiments, the AAV particle of the present disclosure comprising a nucleic acid encoding a protein payload comprises an AAV capsid that allows for transmission across the blood brain barrier after intravenous administration.

In certain embodiments, the composition comprising the AAV particles of the present disclosure is administered to the central nervous system of the subject via systemic administration. In certain embodiments, the systemic administration is intravenous injection.

In certain embodiments, the composition comprising the AAV particles of the present disclosure is administered to the central nervous system of the subject. In certain embodiments, the composition comprising the AAV particles of the present disclosure is administered to a tissue of a subject (e.g., brain of the subject).

In certain embodiments, the composition comprising the AAV particles of the present disclosure is administered to the central nervous system of the subject via intraparenchymal injection. Non-limiting examples of intraparenchymal injections comprise Intrathalamic, intrastriatal, intrahippocampal or targeting the entorhinal cortex.

In certain embodiments, the composition comprising the AAV particles of the present disclosure is administered to the central nervous system of the subject via intraparenchymal injection and intrathecal injection.

In certain embodiments, the AAV particles of the present disclosure may be delivered into specific types of targeted cells, comprising, but not limited to, hippocampal, cortical, motor or entorhinal neurons; glial cells comprising oligodendrocytes, astrocytes and microglia; and/or other cells surrounding neurons such as T cells.

In certain embodiments, the AAV particles of the present disclosure may be delivered to neurons in the striatum (e.g. putamen) and/or cortex.

In certain embodiments, the AAV particles of the present disclosure may be used as a therapy for neurological disease.

In certain embodiments, the AAV particles of the present disclosure may be used to increase target protein and reduce symptoms of neurological disease in a subject. The increase of target protein and/or the reduction of symptoms of neurological disease may he, independently, altered (increased for the production of target protein and reduced for the symptoms of neurological disease) by 5%, 10%, 15%, 20%, 25%, 30%, 35% 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95% 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

### Use of AAV Particles Comprising RNAi Polynucleotides

Provided in the present disclosure are methods for introducing the AAV particles, comprising a nucleic acid sequence encoding the siRNA molecules of the present disclosure into cells, the method comprising introducing into said cells any of the vectors in an amount sufficient for degradation of a target mRNA to occur, thereby activating target-specific RNAi in the cells. In some aspects, the cells may be muscle cells, stem cells, neurons such as but not limited to, motor, hippocampal, entorhinal, thalamic or cortical neurons, and ghat cells such as astrocytes or microglia.

Disclosed in the present disclosure are methods for treating neurological diseases associated with dysfunction of a target protein in a subject in need of treatment. The method optionally comprises administering to the subject a therapeutically effective amount of a composition comprising AAV particles comprising a nucleic acid sequence encoding the siRNA molecules of the present disclosure. As a non-limiting example, the siRNA molecules can silence target gene expression, inhibit target protein production, and reduce one or more symptoms of neurological disease in the subject such that the subject is therapeutically treated.

In certain embodiments, the composition comprising the AAV particles of the present disclosure comprising a nucleic acid sequence encoding siRNA molecules comprise an AAV capsid that allows for transmission across the blood brain barrier after intravenous administration.

In certain embodiments, the composition comprising the AAV particles comprising a nucleic acid sequence encoding the siRNA molecules of the present disclosure is administered to the central nervous system of the subject. In certain embodiments, the composition comprising the AAV particles comprising a nucleic acid sequence encoding the siRNA molecules of the present disclosure is administered to a tissue of a subject (e.g., brain of the subject).

In certain embodiments, the composition comprising the AAV particles comprising a nucleic acid sequence encoding the siRNA molecules of the present disclosure is administered to the central nervous system of the subject via systemic administration. In certain embodiments, the systemic administration is intravenous injection.

In certain embodiments, the composition comprising the AAV particles comprising a nucleic acid sequence encoding the siRNA molecules of the present disclosure is administered to the central nervous system of the subject via intraparenchymal injection. Non-limiting examples of intraparenchymal injections comprise intrathalamic, intrastriatal, intrahippocampal or targeting the entorhinal cortex.

In certain embodiments, the composition comprising the AAV particles comprising a nucleic acid sequence encoding; the siRNA molecules of the present disclosure is administered to the central nervous system of the subject via intraparenchymal injection and intrathecal injection.

In certain embodiments, the AAV particles comprising a nucleic acid sequence encoding the siRNA molecules of the present disclosure may be delivered into specific types of targeted cells, comprising, but not limited to, hippocampal, cortical, motor or entorhinal neurons; glial cells comprising oligodendrocytes, astrocytes and microglia; and/or other cells surrounding neurons such as T cells.

In certain embodiments, the AAV particles comprising a nucleic acid sequence encoding the siRNA molecules of the present disclosure may be delivered to neurons in the striatum and/or cortex.

In certain embodiments, the AAV particles comprising a nucleic acid sequence encoding the siRNA molecules of the present disclosure may be used as a therapy for neurological disease.

In certain embodiments, the AAV particles comprising a nucleic acid sequence encoding the siRNA molecules of the present disclosure may be used as a therapy for Amyotrophic Lateral Sclerosis.

In certain embodiments, the AAV particles comprising a nucleic acid sequence encoding the siRNA molecules of the present disclosure may be used as a therapy for Huntington's Disease.

In certain embodiments, the AAV particles comprising a nucleic acid sequence encoding the siRNA molecules of the present disclosure may be used as a therapy for Parkinson's Disease.

In certain embodiments, the AAV particles comprising a nucleic acid sequence encoding the siRNA molecules of the present disclosure may be used as a therapy for Friedreich's Ataxia.

In certain embodiments, the AAV particles comprising a nucleic acid sequence encoding the siRNA molecules of the present disclosure may be used to suppress a target in order to treat neurological disease. Target protein in astrocytes may be suppressed by 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-6.5%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%, 25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, -80%, -85%, 70-90%, 70-95%, 75-85%, 75-90%, 75-95%, 80-90%, 80-95%, or 90-95%. Target protein in astrocytes may be reduced may be 5%, 10%, 15%, 20%, 25%, 30%, 35%, 40%, 45%, 50%, 55%, 60%, 65%, 70%, 75%, 80%, 85%, 90%, 95%, or more than 95%, 5-15%, 5-20%, 5-25%, 5-30%, 5-35%, 5-40%, 5-45%, 5-50%, 5-55%, 5-60%, 5-65%, 5-70%, 5-75%, 5-80%, 5-85%, 5-90%, 5-95%, 10-20%, 10-25%, 10-30%, 10-35%, 10-40%, 10-45%, 10-50%, 10-55%, 10-60%, 10-65%, 10-70%, 10-75%, 10-80%, 10-85%, 10-90%, 10-95%, 15-25%, 15-30%, 15-35%, 15-40%, 15-45%, 15-50%, 15-55%, 15-60%, 15-65%, 15-70%, 15-75%, 15-80%, 15-85%, 15-90%, 15-95%, 20-30%, 20-35%, 20-40%, 20-45%, 20-50%, 20-55%, 20-60%, 20-65%, 20-70%, 20-75%, 20-80%, 20-85%, 20-90%, 20-95%, 25-35%, 25-40%, 25-45%, 25-50%, 25-55%, 25-60%, 25-65%, 25-70%,25-75%, 25-80%, 25-85%, 25-90%, 25-95%, 30-40%, 30-45%, 30-50%, 30-55%, 30-60%, 30-65%, 30-70%, 30-75%, 30-80%, 30-85%, 30-90%, 30-95%, 35-45%, 35-50%, 35-55%, 35-60%, 35-65%, 35-70%, 35-75%, 35-80%, 35-85%, 35-90%, 35-95%, 40-50%, 40-55%, 40-60%, 40-65%, 40-70%, 40-75%, 40-80%, 40-85%, 40-90%, 40-95%, 45-55%, 45-60%, 45-65%, 45-70%, 45-75%, 45-80%, 45-85%, 45-90%, 45-95%, 50-60%, 50-65%, 50-70%, 50-75%, 50-80%, 50-85%, 50-90%, 50-95%, 55-65%, 55-70%, 55-75%, 55-80%, 55-85%, 55-90%, 55-95%, 60-70%, 60-75%, 60-80%, 60-85%, 60-90%, 60-95%, 65-75%, 65-80%, 65-85%, 65-90%, 65-95%, 70-80%, 70-85%, 70-90%, 70-95%, 75-85%, 75-90%, 75-95%, 80-90%, 80-95%, or 90-95%.

In certain embodiments, administration of the AAV particles encoding a siRNA of the present disclosure, to a subject may lower target protein levels in a subject. The target protein levels may be lowered by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS of a subject. As a non-limiting, example, the AAV particles may lower the protein levels of a target protein by at least 50%, As a non-limiting example, the AAV particles may lower the proteins levels of a target protein by at least 40%.

### Therapeutic Indications

### Parkinson s Disease

Parkinson's Disease (PD) is a progressive disorder of the nervous system affecting especially the substantia nigra of the brain. PD develops are a result of the loss of dopamine producing brain cells. Typical early syinptoms of PD comprise shaking or trembling of a limb, e.g, hands, arms, legs, feet and face. Additional characteristic symptoms are stiffness of the limbs and torso, slow movement or an inability to move, impaired balance and coordination, cognitional changes, and psychiatric conditions e.g., depression and visual hallucinations. PD has both familial and idiopathic forms and it is suggestion to be involved with genetic and environmental causes. PD affects more than 4 million people worldwide. In the US, approximately 60,000 cases are identified annually. Generally, PD begins at the age of 50 or older. An early-onset form of the condition begins at age younger than 50, and juvenile-onset PD begins before age of 20.

Death of dopamine producing brain cells related to PD has been associated with aggregation, deposition and dysfunction of alpha-synuclein protein (see, e.g. Marques and Outeiro, 2012, Cell Death Dis. 3:e350, Jenner, 1989, J Neurol Neurosurg Psychiatry. Special Supplement, 22-28, and references therein). Studies have suggested that alpha-synuclein has a role in presynaptic signaling, membrane trafficking and regulation of dopamine release and transport. Alpha-synuclein aggregates, e,g. in forms of oligomers, have been suggested to he species responsible for neuronal dysfunction and death. Mutations of the alpha-synuclein gene (SNCA) have been identified in the familial forms of PD, but also enviromnental factors, e.g. neurotoxin affect alpha-synuclein aggregation. Other suggested causes of brain cell death in PD are dysfunction of proteosomal and lysosomal systems, reduced mitochondrial activity.

PD is related to other diseases related to alpha-synuclein aggregation, referred to as “synucleinopathies.” Such diseases comprise, but are not limited to, Parkinson's Disease Dementia (PDD), multiple system atrophy (MSA), dementia with Lewy bodies, juvenile-onset generalized neuroaxonal dystrophy (Hallervorden-Spatz disease), pure autonomic failure (PAF), neurodegeneration with brain iron accumulation type-1 (NBIA-1) and combined Alzheimer's and Parkinson's disease.

As of today, no cure or prevention therapy for PD has been identified. A variety of drug therapies available provide relief to the symptoms. Non-limiting examples of symptomatic medical treatments comprise carbidopa and levodopa combination reducing stiffness and slow movement, and anticholinergics to reduce trembling and stiffness. Other optional therapies comprise e.g. deep brain stimulation and surgery. There remains a need for therapy affecting the underlying pathophysiology. For example, antibodies targeting alpha-synuclein protein, or other proteins relevant for brain cell death in PD, may be used to prevent and/or treat PD.

In certain embodiments, methods of the present disclosure may be used to treat subjects suffering from PD and other synucleinopathies. In certain embodiments, methods of the present disclosure may be used to treat subjects suspected of developing PD and other synucleinopathies.

AAV particles, pharmaceutical formulations and methods of using the viral particles described in the present disclosure may be used to prevent, manage and/or treat PD.

### Spinal Muscular Atrophy

Spinal muscular atrophy (SMA) is a hereditary disease-causing weakness and wasting of the voluntary muscles in the arms and legs of infants and children. SMA is associated with abnormalities in the protein production of the survival motor neuron gene 1 (SMN1). Lack of the protein affects degeneration and death of lower motor neurons. Typical symptoms comprise floppy limbs and trunk, feeble movement of the arms and legs, difficulties in swallowing and eating, and impaired breathing. SMA is the most common genetic disorder leading to death of children under 2 years of age. SMA affects one in 6,000 to 10,000 people.

As of today, there is no cure for SMA. Therapies available are aimed at management of the symptoms and prevention of additional complications. Such therapies are associated e.g. with cardiology, movement management, respiratory care and mental health. There remains a need for therapy affecting the underlying pathophysiology of SMA and related diseases and ailments.

In certain embodiments, the AAV particles and methods of the present disclosure may be used to treat subjects suffering from SMA and related diseases and ailments. In certain embodiments, methods of the present disclosure may be used to treat subjects suspected of developing SMA or related diseases and ailments.

AAV particles, pharmaceutical formulations and methods of using the viral particles described in the present disclosure may be used to prevent, manage and/or treat SMA and related diseases and ailments.

### Alzheimer's Disease

Alzheimer's Disease (AD) is a debilitating neurodegenerative disease and the most common form of dementia affecting the memory, thinking and behavior. Typical early symptom is difficulty of remembering newly learned information. As the disease advances, symptoms comprise disorientation, changes in sleep, changes in mood and behavior, confusion, unfound suspicions and eventually difficulty to speak, swallow and walk. AD currently afflicts more than 35 million people worldwide, with that number expected to double in coming decades.

As of today, no cure or prevention therapy for AD has been identified. Drug therapy to treat memory loss, behavioral changes and sleep changes, and to slow down the progression of AD are available. However, these symptomatic treatments do not address the underlying pathophysiology.

In certain embodiments, methods of the present disclosure may be used to treat subjects suffering from AD and related diseases and ailments. In certain embodiments, methods of the present disclosure may be used as a therapy for to treat subjects suspected of developing AD or related diseases and ailments.

AAV particles, pharmaceutical formulations and methods of using the viral particles described in the present disclosure may be used to prevent, manage and/or treat AD and related diseases and ailments.

### Amyotrophic Lateral Sclerosis

Amyotrophic Lateral Sclerosis (ALS), also known as Lou Gehrig's disease or classical motor neuron disease, is a rapidly progressive and fatal neurological disease. ALS is associated with cell degeneration and death of the upper and lower motor neurons, leading to enablement of muscle movement, weakening, wasting and loss of control over voluntary muscle movement. Early symptoms comprise muscle weakness of hands, legs and swallowing muscles, eventually progressing to inability to breathe due to diaphragm failure. According to Centers for Disease Control and Prevention (CDC), ALS affects an estimated 12,000-15,000 individuals in the US. About 5-10% of cases are familial.

ALS, as other non-infectious neurodegenerative diseases, has been characterized by presence of misfolded proteins. Familial ALS has been associated with mutations of TAR DNA-binding protein 43 (TDP-43) and RNA-binding protein FUS/TLS. Some proteins have been identified to slow down progression of ALS, such as, but not limited, to growth factors, e.g. insulin-like growth factor 1 (IGF-1), glial cell line-derived growth factor, brain-derived growth factor, vascular endothelial growth factor and ciliary neurotrophic factor, or growth factors promoting muscle growth, e.g. myostatin.

As of today, there is no prevention or cure for ALS. FDA approved drug niluzole has been approved to prolong the life but does not have an effect on symptoms. Additionally, drugs and medical devices are available to tolerate pain and attacks associated with ALS. There remains a need for therapy affecting the underlying pathophysiology.

In certain embodiments, methods of the present disclosure may be used to treat subjects suffering from ALS and related diseases and ailments. In certain embodiments, methods of the present disclosure may be used to treat subjects suspected of developing ALS or related diseases and ailments.

AAV particles, pharmaceutical formulations and methods of using the viral particles described in the present disclosure may be used to prevent, manage and/or treat ALS and related diseases and ailments.

### Huntington's Disease

Huntington's disease (HD) is a rare, inherited disorder causing degeneration of neurons in the motor control region of the brain, as well as other areas. Typical symptoms of the disease comprise uncontrolled movements (chorea), abnormal postures, impaired coordination, slurred speech and difficulty of feeding and swallowing accompanied by changes in behavior, judgment and cognition. HD is caused by mutations in the gene associated with the huntingtin (HTT) protein. The mutation causes the (CAG) blocks of DNA to repeat abnormally many times. HD affects approximately 30,000 individuals in the US.

HD is characterized by mutations of the huntingtin (HTT) protein with abnormal expansions of polyglutamine tracts, e.g. expansion of the length of glutamine residues encoded by CAG repeats. The expansion threshold for occurrence of the disease is considered to be approximately 35-40 residues. HD is also associated with beta sheet rich aggregates in striatal neurons formed by N-terminal region of HTT. The expansions and aggregates lead to gradual loss of neurons as HD progresses. Additionally, the cell death in FED is associated with death receptor 6 (DR6) which is known to induce apoptosis.

As of today, there is no therapy to cure, or prevent the progression of the disease. Drug therapies available are aimed at management of the symptoms. For example, FDA has approved tetrabenezine to be prescribed for prevention of chorea. Additionally, e.g. antipsychotic drugs may help to control delusions, hallucinations and violent outbursts. There remains a need for therapy affecting the underlying, pathophysiology, such as antibody therapies targeting the HTT protein, DR6 protein, and/or other HD associated proteins.

In certain embodiments, methods of the present disclosure may be used to treat subjects suffering from HD and related diseases and ailments. In certain embodiments, methods of the present disclosure may he used to treat subjects suspected of developing HD or related diseases and ailments.

AAV particles, pharmaceutical formulations and methods of using the viral particles described in the present disclosure may be used to prevent, manage and/or treat HD and related diseases and ailments.

### VI. DEFINITIONS

At various places in the present disclosure, substituents or properties of compounds of the present disclosure are disclosed in groups or in ranges. It is specifically intended that the present disclosure comprise each and every individual or sub-combination of the members of such groups and ranges.

Unless stated otherwise, the following terms and phrases have the meanings described below. The definitions are not meant to be limiting in nature and serve to provide a clearer understanding of certain aspects of the present disclosure.

Adeno-associated virus: The term “adeno-associated virus” or “AAV” as used herein refers to members of the dependovirus genus comprising any particle, sequence, gene, protein, or component derived therefrom.

AAV Particle: As used herein, an “AAV particle” is a virus which comprises a capsid and a viral genome with at least one payload region and at least one ITR region. AAV particles of the present disclosure may be produced recombinantly and may be based on adeno-associated virus (AAV) parent or reference sequences. AAV particle may be derived from any serotype, described herein or known in the art, comprising combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions of the present disclosure may have activity and this activity may involve one or more biological events.

Administering: As used herein, the term “administering” refers to providing a pharmaceutical agent or composition to a subject.

Administered in combination: As used herein, the term “administered in combination” or “combined administration” means that two or more agents are administered to a subject at the same time or within an interval such that there may be an overlap of an effect of each agent on the patient. In certain embodiments, they are administered within about 60, 30, 15, 10, 5, or 1 minute of one another. hi certain embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration comprises the reduction of neuron loss.

Animal: As used herein, the term “animal” refers to any member of the animal kingdom. In certain embodiments, “animal” refers to humans at any stage of development. in certain embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In certain embodiments, animals comprise, but are not limited to, mammals, birds, reptiles, amphibians, fish, and worms. In certain embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. As used herein, the term “about” means +/−10% of the recited value. In certain embodiments, the term “approximately” refers to a ranae of values that fall within 25%, 20%, 19%, 18%, 17%, 16%, 15%, 14%, 13%, 12%, 11%, 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Baculoviral expression vector (BEV): As used herein a BEV is a baculoviral expression vector, i.e., a polynucleotide vector of baculoviral origin. Systems using BEVs are known as baculoviral expression vector systems (BEVSs),

mBEV or modified BEV: As used herein, a modified BEV is an expression vector of baculoviral origin which has been altered from a starting BEV (whether wild type or artificial) by the addition and/or deletion and/or duplication and/or inversion of one or more: genes; gene fragments; cleavage sites; restriction sites; sequence regions; sequence(s) encoding a payload or gene of interest; or combinations of the foregoing.

Bifunctional: As used herein, the term “bifunctional” refers to any substance, molecule or moiety which is capable of or maintains at least two functions. The functions may affect the same outcome or a different outcome. The structure that produces the function may be the same or different.

BIIC: As used herein a BIIC is a baculoviral infected insect cell.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, an AAV particle of the present disclosure may be considered biologically active if even a portion of the encoded payload is biologically active or mimics an activity considered biologically relevant.

Capsid: As used herein, the term “capsid” refers to the protein shell of a virus particle.

Codon optimized: As used herein, the terms “codon optimized” or “codon optimization” refers to a modified nucleic acid sequence which encodes the same amino acid sequence as a parent/reference sequence, but which has been altered such that the codons of the modified nucleic acid sequence are optimized or improved for expression in a particular system (such as a particular species or group of species). As a non-limiting example, a nucleic acid sequence which comprises an AAV capsid protein can be codon optimized for expression in insect cells or in a particular insect cell such Spodoptera frugiperda cells. Codon optimization can be completed using methods and databases known to those in the art.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other, For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity, As used herein, the term “substantially complementary” means that the siRNA has a sequence (e.g., in the antisense strand) which is sufficient to bind the desired target mRNA, and to trigger the RNA silencing of the target mRNA.

Compound: Compounds of the present disclosure comprise all of the isotopes of the atoms occurring in the intermediate or final compounds. “Isotopes” refers to atoms having the same atomic number but different mass numbers resulting; from a different number of neutrons in the nuclei. For example, isotopes of hydrogen comprise tritium and deuterium.

The compounds and salts of the present disclosure can be prepared in combination with solvent or water molecules to form solvates and hydrates by routine methods.

Conditionally active: As used herein, the term “conditionally active” refers to a mutant or variant of a wild-type polypeptide, wherein the mutant or variant is more or less active at physiological conditions than the parent polypeptide. Further, the conditionally active polypeptide may have increased or decreased activity at aberrant conditions as compared to the parent polypeptide. A conditionally active polypeptide may be reversibly or irreversibly inactivated at normal physiological conditions or aberrant conditions.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In certain embodiments, two or more sequences are said to be “completely conserved” if they are 100% identical to one another. In certain embodiments, two or more sequences are said to be “highly conserved” if they are at least 70% identical, at least 80% identical, at least 90% identical, or at least 95% identical to one another. In certain embodiments, two or more sequences are said to be “highly conserved” if they are about 70% identical, about 80% identical, about 90% identical. about 95%, about 98%, or about 99% identical to one another. In certain embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. in certain embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide, or polypeptide or may apply to a portion, region or feature thereof.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“TRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Controlled Release: As used herein, the term “controlled release” refers to a pharmaceutical composition or compound release profile that conforms to a particular pattern of release to affect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering an AAV particle, a compound, substance, entity, moiety, cargo or payload.

Delivery Agent: As used herein, “delivery agent” refers to any substance which facilitates, at least in part, the in vivo delivery of an AAV particle to targeted cells.

Destabilized: As used herein, the term “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art comprising radiography, fluorescence, chemiluminescence, enzymatic activity, absorbance and the like. Detectable labels comprise radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments of the present disclosure are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicirm, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” comprises at least one AAV particle and a delivery agent or excipient.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may he of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In certain embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% for at least one stretch of at least about 20 amino acids. In certain embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

Heterologous Region: As used herein the term “heterologous region” refers to a region which would not be considered a homologous region.

Homologous Region: As used herein the term “homologous region” refers to a region which is similar in position, structure, evolution origin, character, form or function.

Identity: As used herein, the term “identity” refers to the overall relatedness between polymeric molecules, e.g., between polynucleotide molecules e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: informatics and Genome Projects, Smith, D. ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of which are each incorporated herein by reference in their entireties, insofar as they do not conflict with the present disclosure. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences comprise, but are not limited to those disclosed in Carillo, H., and Lipman, D., SIAM J Applied Math., 48:1073 (1988); the contents of which are each incorporated herein by reference in their entireties, insofar as they does not conflict with the present disclosure. Techniques for determining identity are codified in publicly available computer programs. Exemplary computer software to determine homology between two sequences comprise, but are not limited to, GCG program package, Devereux, J., et al., Nucleic Acids Research, 12(1), 387 (1984)), BLASTP, BLASTN, and PASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA. transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In certain embodiments, isolated agents are more than about 80%, about 85%, about 90%, about 91%, about 92%, about 93%, about 94%, about 95%, about 96%, about 97%, about 98%, about 99%, or more than about 99% pure, As used herein, a substance is “pure” if it is substantially free of other components. As used herein, the terra “substantially isolated” is meant that a substance is substantially separated from the environment in which it was formed or detected. Partial separation can comprise, for example, a composition enriched in the substance or AAV particles of the present disclosure. Substantial separation can comprise compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein “linker” refers to a molecule or group of molecules which connects two molecules. A linker may be a nucleic acid sequence connecting two nucleic acid sequences encoding two different polypeptides. The linker may or may not be translated, The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule of the present disclosure. Molecules may be modified in many ways comprising chemically, structurally, and functionally. As used herein, embodiments of the disclosure are “modified” when they have or possess a feature or property, whether structural or chemical, that varies from a starting point, wild type or native molecule.

Mutation: As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes.

Naturally Occurring: As used herein, “naturally occurring” or “wild-type” means existing in nature without artificial aid, or involvement of the hand of man.

Non-human vertebrate: As used herein, a “non-human vertebrate” comprises all vertebrates except Homo sapiens, comprising wild and domesticated species. Examples of non-human vertebrates comprise, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic Acid: As used herein, the term “nucleic acid”, “polynucleotide” and “oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms comprise double- and single-stranded DNA, as well as double- and single stranded RNA.

Off-target: As used herein, “off target” refers to any unintended effect on any one or more taraet, gene, or cellular transcript.

Open reading frame: As used herein, “open reading frame” or “ORF” refers to a sequence which does not contain a stop codon within the given reading frame, other than at the end of the reading frame.

Operably linked: As used herein, the phrase “operably linked” refers to a functional connection between two or more molecules, constructs, transcripts, entities, moieties or the like. As a non-limiting example, a promoter is “operably linked” to a nucleotide sequence when the promoter sequence controls and/or regulates the transcription of the nucleotide sequence.

Patient: As used herein, “patient” refers to a subject who may seek or need treatment, requires treatment, is receiving treatment, will receive treatment, or a subject who is under care by a trained professional for a particular disease or condition.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, or a modulatory nucleic acid or regulatory nucleic acid.

Payload construct: As used herein, “payload construct” is one or more vector construct which comprises a polynucleotide region encoding or comprising a payload that flanked on one or both sides by an inverted terminal repeat (ITR) sequence. The payload construct presents a template that is replicated in a viral production cell to produce a therapeutic viral genome.

Payload construct vector: As used herein, “payload construct vector” is a vector encoding or comprising a payload construct, and regulatory regions for replication and expression of the payload construct in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Peptide: As used herein, “peptide” is less than or equal to 50 amino acids long, e.g., about 5, 10, 15, 20, 25, 30, 35. 40, 45, or 50 amino acids long.

Pharmaceutically acceptable: The phrase “pharmaceutically acceptable” employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. 111901 Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may comprise, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients comprise, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also comprises pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts comprise, but are not limited to, mineral or organic acid. salts of basic residues such as amines; alkali or organic salts of acidic residues such as carboxylic acids; and the like. Representative acid addition salts comprise acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, laurel sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts comprise sodium, lithium, potassium, calcium, magnesium, and the like, as well as nontoxic ammonium, quaternary ammonium, and amine cations, comprising, but not limited to ammonium, tetramethvlammonium, tetraethylammonium, methylamine, dimethylamine, trimethylamine, triethyla.mine, ethylamine, and the like. The pharmaceutically acceptable salts of the present disclosure comprise the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile can be used. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), the contents of which are each incorporated herein by reference in their entireties, insofar as they do not conflict with the present disclosure.

Pharmaceutically acceptable solvate: The term “pharmaceutically acceptable solvate,” as used herein, means a compound of the present disclosure wherein molecules of a suitable solvent are incorporated in the crystal lattice. A suitable solvent is physiologically tolerable at the dosage administered. For example, solvates may be prepared by crystallization, recrystallization, or precipitation from a solution that comprises organic solvents, water, or a mixture thereof. Examples of suitable solvents are ethanol, water (for example, mono-, di-, and tri-hydrates)), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas comprising the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue,

Physicochemical: As used herein, “physicochemical” means of or relating to a physical and/or chemical property.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” comprise those provided herein and fragments, mutants, variants, and alterations thereof.

Proximal: As used herein, the term “proximal” means situated nearer to the center or to a point or region of interest.

Purified: As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In certain embodiments, when referring to a protein or protein module, a region may comprise a linear sequence of amino acids along the protein or protein module or may comprise a three-dimensional area, an epitope and/or a cluster of epitopes. In certain embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In certain embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In certain embodiments, N-terminal regions may comprise any length of amino acids that comprises the N-terminus but does not comprise the C-terminus. In certain embodiments, C-terminal regions may comprise any length of amino acids, which comprise the C-terminus, but do not comprise the N-terminus.

In certain embodiments, when referring to a polynucleotide, a region may comprise a linear sequence of nucleic acids along the polynucleotide or may comprise a three-dimensional area, secondary structure, or tertiary structure. In certain embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In certain embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In certain embodiments, 5′ regions may comprise any length of nucleic acids that comprises the 5′ terminus but does not comprise the 3′ terminus. In certain embodiments, 3′ regions may comprise any length of nucleic acids, which comprise the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering, or RATAi: As used herein, the term “RNA interfering” or “RNAI” refers to a sequence specific regulatory mechanism mediated by RNA molecules which results in the inhibition or interfering or “silencing” of the expression of a corresponding protein-coding gene. RNAi has been observed in many types of organisms, comprising plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs),

Sample: As used herein, the term “sample” or “biological sample” refers to a subset of its tissues, cells or component parts (e.g. body fluids, comprising but not limited to blood, mucus, lymphatic fluid, synovial fluid, cerebrospinal fluid, saliva, amniotic fluid, amniotic cord blood, urine, vaginal fluid and semen). A sample further may comprise a homogenate, lysate or extract prepared from a whole organism or a subset of its tissues, cells or component parts, or a fraction or portion thereof, comprising but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle comprised of at least two components, a protein capsid and a polynucleotide sequence encoding a self-complementary genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” comprises a siRNA strand having sufficient complementarity to a section of about 10-50 nucleotides of the mRNA of the gene targeted for silencing and a siRNA strand haying sufficient complementarity to form a duplex with the other siRNA strand.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. In certain embodiments, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24. nucleotides (or nucleotide analogs). The term “short” siRNA refers to a siRNA comprising 5-23 nucleotides, such as 21 nucleotides (or nucleotide analogs), for example, 19, 20, 21 or 22 nucleotides. The term “long” siRNA refers to a siRNA comprising 24-60 nucleotides, such as about 24-25 nucleotides, for example, 23, 24, 25 or 26 nucleotides. Short siRNAs may, in some instances, comprise fewer than 19 nucleotides, e.g., 16, 17 or 18 nucleotides, or as few as 5 nucleotides, provided that the shorter siRNA retains the ability to mediate RNAi. Likewise, long siRNAs may, in some instances, comprise more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules ss-siRNAs) or double stranded RNA molecules (ds-siRNAs) comprising a sense strand and an antisense strand which hybridized to form a duplex structure called siRNA duplex.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event. In certain embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and in certain embodiments, capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the present disclosure may he administered, e.g., for experimental, diagnostic, prophylactic, and/or therapeutic purposes. Typical subjects comprise animals (e.g., mammals such as mice, rats, rabbits, non-human primates, and humans) and/or plants. The subject or patient may seek or need treatment, require treatment, is receiving treatment, will receive treatment, or is under care by a trained professional for a particular disease or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used. herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In certain embodiments, an individual who is susceptible to a disease, disorder, and/or condition (fir example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In certain embodiments, an individual who is susceptible to a disease, disorder, and/or condition will develop the disease, disorder, and/or condition. In certain embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time,

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may he an animal, such as a mammal, a human, or a human patient.

Terminal region: As used herein, the term “terminal region” refers to a region on the 5′ or 3′ end of a region of linked nucleosides or amino acids (polynucleotide or polypeptide, respectively).

Terminally optimized: The term “terminally optimized” when referring to nucleic acids means the terminal regions of the nucleic acid are improved in some way, e.g., codon optimized, over the native or wild type terminal regions.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. in certain embodiments, a therapeutically effective amount is provided in a single dose. In certain embodiments, a therapeutically effective amount is administered in a dosage regimen comprising a plurality of doses. Those skilled in the art will appreciate that in certain embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24-hour period. It may be administered as a single unit dose.

Transfection: As used herein, the term “transfection” refers to methods to introduce exogenous nucleic acids into a cell. Methods of transfection comprise, but are not limited to, chemical methods, physical treatments and cationic lipids or mixtures,

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may comprise adeno-associated virus (AAV) parent or reference sequence. Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, which sequence may he wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral genome: As used herein, a “viral genome” or “vector genome” refers to the nucleic acid sequence(s) encapsulated in an AAV particle.

### VII. EQUIVALENTS AND SCOPE

Those skilled in the art will recognize or be able to ascertain using no more than routine experimentation, many equivalents to the specific embodiments in accordance with the present disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that comprise “or” between one or more members of a group are considered satisfied if one, more than one, or all of the group members are present in, employed in, or otherwise relevant to a given product or process unless indicated to the contrary or otherwise evident from the context. The present disclosure comprises embodiments in which exactly one member of the group is present in, employed in, or otherwise relevant to a given product or process. The present disclosure comprises embodiments in which more than one, or the entire group members are present in, employed in, or otherwise relevant to a given product or process,

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are comprised. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

All publications, patent applications, patents, and other references mentioned herein are incorporated by reference in their entirety. In case of conflict, the present specification, comprising definitions, will control. In addition, section headings, the materials, methods, and examples are illustrative only and not intended to be limiting.

### VIII. EXAMPLES

### Example 1. Production of Source Rep/Cap BIICs (A)

### CP BEE Pool

One vial of the Sf9 CB was thawed in a 125 mL shaker flask (37° C. using waterbath, 1-5 minutes until ice crystals dissipate), and then diluted into 19-20 mL working volume of Hvclone SFX Insect Cell Culture Media. The shaker flask was incubated at 27° C. (135 rpm shaking, 0% v/v of CO₂) in a first expansion (P0, 3-4 days) until the cell density of the Sf9 cell mixture was expanded to 4.0×10⁶ cells/mL.

The cell mixture was then seeded and expanded through multiple additional expansion steps using larger shaker flasks, with a target output density of 4.0×10⁶ cells/mL for each expansion step to allow for a consistent seeding density of 0.5-1.5×10⁶ cells/mL in subsequent expansion steps. Expansions were completed at 27° C. for 3-5 days (0% v/v of CO₂) with 135 rpm shaking (≤2 L working volume) or 90 rpm shaking (>2 L working volume). The following additional expansions were completed: (i) expansion up to 200 mL working volume in a 1.0 L flask; and (ii) expansion up to 1000 mL working volume in a 3 L flask.

A Rep/Cap Transfection Mixture was prepared by combining 5 μg of Rep/Cap Bacmid. material with 375 μL of WFI water. The diluted Bacmid mixture was combined with 30 μL of Promega FuGENE HD (Transfection Agent) and an additional 345 μL of WFI water, and then incubated at 27° C. for 15 minutes to provide a Transfection Cocktail.

25 mL of expanded Sf9 cell mixture was seeded into a 125 mL flask (1.0×10⁶ cells/mL seeding concentration) and expanded to a target infection density of 2.5-4.0×10⁶ cells/mL. The Transfection Cocktail was added to the 125 mL flask and incubated at 27° C. for 5-7 days (0% v/v of CO₂, 135 rpm agitation). The resulting mixture was centrifuged in 50 mL conical tubes for 5 minutes, and the supernatant containing P1 BEVs was collected and. pooled with other PI BEV supernatants, The P1 BEV pool was stored at 5° C.

Expanded Sf9 cell mixture was seeded into a Cellstar 6-well Cell Polystyrene Culture Plate (2 mL per well, 0.5-1.0×10⁶ cells/mL seeding concentration) with gentle rocking to evenly distribute cells, followed by incubation at 27° C. for 90 minutes (0% v/v of CO₂, 0 rpm agitation). P1 BEVs were serial diluted with 1-lyclone SFX Insect Cell Culture Media to a target dilution of 1.0×10⁷ BEVs/mL, and then 1 mL of diluted P1 BEV mixture was added to each well with gentle rocking to evenly distribute P1 BEVs. The infection mixture was incubated at 27° C. for 90 minutes (0% v/v of CO₂, 0 rpm agitation),

Agarose gel was prepared by combining 4% w/v agarose 1:3 with Life Technologies Sf-900 Medium overlay (melt agarose at 70° C., cool to 37° C. for combination). 2 mL of Agarose Overlay was then added to each well, and the plates were maintained at room temperature for 15-20 minutes for the agarose gel to harden. Overlaid plates were then incubated at 27° C. for 5-14 days (0% v/v of CO₂, 0 rpm agitation) until plaque formation was observed. Plaques in each well were processed through testing and Plaque Picking to provide a single Plaque for Clonal Plaque Purification (i.e. Single Plaque Expansion). The Single Plaque was expanded using SIP cell mixture and incubated at 27° C. for 3-5 days (0% v/v of CO₂, 0 rpm. agitation). The resulting CPI BEVs were harvested using centrifugation in 50 mL conical tubes for 5 minutes and collection of supernatant containing CP1 BEVs into a CP1 BEV pool.

### BEV Infection/BIIC Production

Sf9 cell mixture was seeded and expanded through multiple expansion steps using larger shaker flasks, with a target output density of 4.0×10⁶ cells/mL for each expansion step to allow for a consistent seeding density of 0.5-1.0×10⁶ cells/mL in subsequent expansion steps. Expansions were completed at 27° C. for 3-5 days (0% v/v of CO₂) with 135 rpm shaking (≤2 L working volume) or 90 rpm shaking (>2 L working volume). The following expansions were completed: (i) expansion up to 200 mL working volume in a 1.0 L (ii) expansion up to 1000 mL working volume in a 3 L flask; and (iii) expansion up to 2500 mL working volume in a 5 L flask, with a final output density of 2.0×10⁶ cells/mL.

200 mL of expanded Sf9 cell mixture was seeded into a 1.0 L flask (1.0×10⁶ cells/mL seeding density) and expanded to a viable infection cell density of ≥2.0×10⁶ cells/mL, and then infected with 0.01 MOI of CP1 BEV. Infected cells were then incubated and expanded at 27° C. for 48-80 hours (0% v/v of CO₂, 135 rpm) until cells reach ≥2.0×10⁶ cells/mL (VCD), ≥16.5 μm cell diameter and ≥75% cell viability. The infected cells were harvested by spinning down (polypropylene centrifuge tubes, 5 min) and resuspending the cell pellet at 4.0×10⁷ cells/mL in Hyclone SFX Insect Cell Culture Media, followed by the addition of 300 mM Trehalose, 14% v/v of DMSO, and additional SEX Culture Media to provide target VCD of 2.0×10⁶ cells/mL. Rep/Cap Source BIICs were aliquoted into 2 mL or 5 mL cryovials and frozen down to ≤−65° C. using control rate freezer, and then stored at −80° C. or in LN₂ vapor.

### Example 2. Production of Source Transgene BIICs (A)

Transgene Source BIICs were produced according to Example 1, with Transgene Bacmid. material used for P1 BEV production instead of Rep/Cap Bacmid material.

### Example 3. Production Source ReplCap BIICs (B)

### CP BEV Pool

One vial of the Sf9 CB was thawed in a 125 mL shaker flask (37° C. using waterbath, 1-5 minutes until ice crystals dissipate), and then diluted into 40 mL working volume of Hyclone SFX Insect Cell Culture Media. The shaker flask was incubated for a first expansion (P0, 3-4 days) until the cell density of the Sf9 cell mixture was expanded to 4.0×10⁶ cells/mL.

The culture was then seeded and expanded through multiple additional expansion steps using larger shaker flasks, with a target output density of 4.0×10⁶ cells/int for each expansion step to allow for a consistent seeding density of 0.5-1.0×10⁶ cells/mL in subsequent expansion steps. Expansions were completed at 27° C. for 3-5 days and comprised: (i) expansion up to 200 mL working volume in a 1.0 L flask (PI); and (ii) expansion up to 1000 mL working volume in a 3 L flask (P2).

A Rep/Cap Transfection Mixture was prepared by combining 30 μg of Rep/Cap Bacmid material with 0.6 mL of ThermtoFisher Grace's insect Media (Transfection Media). The diluted Bacmid mixture was combined with 30 μL of ThermoFisher Cellfectin II Reagent (Transfection Agent) and an additional 0.6 mL of Transfection Media, followed by incubation at 18-25° C. for 25-35 minutes, and then further dilution with 4.8 mL of Transfection Media to provide a Transfection Cocktail.

60 mL of expanded Sf9 cell mixture was seeded into a 125 mL flask and expanded up to 1.0×10⁶ cells/int seeding concentration. The Sf9 cell mixture was then seeded into a 6-well Cell Culture Plate (2 mL per well, 1.0×10⁶ cells/mL seeding concentration). 1 mL of Transfection Cocktail was added to each well, and the plate was incubated at 27° C. for 4-5 hours. 2 mL of Hyclone SFX Insect Cell Culture Media was added to each well, and the plates were then incubated at 27° C. for 3-4 days. The resulting mixtures were centrifuged in 50 mL conical tubes for 5 minutes, and supernatant containing P1 BEVs was collected and pooled with other P1 BEV supernatants. The P1 BEV pool was stored at 4-8° C.

Expanded Sf9 cell mixture was seeded into a 6-well Cell Culture Plate (2 mL per well, 0.5-1.0 cells/mL seeding concentration) with gentle rocking to evenly distribute cells, followed by incubation at 27° C. for 90 minutes. P1 BEVs were serial diluted with Hyclone SFX Insect Cell Culture Media to a target dilution of 1.0-5.0×10⁷ BEVs, and then 1 mL of diluted P1 BEV mixture was added to each well with gentle rocking to evenly distribute P1 BEVs. The infection mixture was incubated at 27° C. for 90 minutes.

Agarose gel was prepared by combining 4% w/v agarose 1:3 with Life Technologies Sf-900 Medium overlay. Agarose Overlay was added to each well, and the plates were maintained at room temperature for 15-20 minutes for the agarose gel to harden. Overlaid plates were then incubated at 27° C. for 10 days until plaque formation was observed. Plaques in each well were processed through testing and Plaque Picking to provide a single Plaque for Clonal Plaque Purification (i.e. Single Plaque Expansion). The Single Plaque was expanded using Std cell mixture of 120 mL pools in 500 mL flask, with incubation at 27° C. for 4 days, The resulting CP2 BEVs were harvested using centrifugation in 50 mL conical tubes for 5 minutes and collection of supernatant containing CP2 BEVs into a CP2 BEV pool.

### BEV Infection/BIIC Production

Sf9 cell mixture was seeded and expanded through multiple expansion steps up to 3000 mL working volume in a 5L flask, with a final infection density of 1.0×10⁶ cells/mL. The expanded Sf9 cell mixture was then infected with 0.01 MO1 of CP2 BEV. Infected cells were incubated and expanded at 27° C. for 48-36 hours, then harvested by spinning down (polypropylene centrifuge tubes, 5 min) and resuspending the cell pellet at 2.0×10⁷ cells/mt in Hyclone SFX Insect Cell Culture Media, followed by the addition of 300 nM Trehalose, 14% v/v of DMSO, and additional SFX Culture Media to provide target VCD of 2.0×10⁶ cells/mL. Rep/Cap Source BIICs were aliquoted into 2 mL or 5 mL cryovials and frozen down to ≤−65° C. using control rate freezer, and then stored at −80° C. or in LN₂ vapor.

### Example 4. Production of Source Transgene BACs (B)

Transgene Source BIICs were produced according to Example 3, with Transgene Bacmid material used for P1 BEV production instead of Rep/Cap Bacmid material.

### Example 5. Production of Infection BIICs from Source BIICs

One vial of Sf9 9f4 CB was thawed in a 125 mL shaker flask (37° C. using waterbath, 1-5 minutes until ice crystals dissipate), and then diluted into 20 mL working volume of ESF-AF culture medium. The shaker flask was incubated at 27° C. (100 rpm shaking, 2-inch orbital diameter) in a non-humidified, ambient air, temperature regulated incubator in a first expansion until the cell density was expanded to between 5.0-8.0×10⁶ cells/mL.

The culture was then seeded and expanded through multiple additional expansion steps using larger shaker flasks, with a target output density of 5.0-8.0×10⁶ cells/mL for each expansion step to allow for a consistent seeding density of 1.0-1.5×10⁶ cells/mi, in subsequent expansion steps. Expansions were completed at 27° C. for 3-5 days with 100 rpm shaking (≤2 L working volume) or 80 rpm shaking (>2 L working volume).

The following additional expansions were completed: (i) expansion up to 100 mL working volume in a 500mL flask; (ii) expansion up to 400 mL working volume in a 1.0 L flask; (iii) expansion up to 1500 mL working volume in a 3 L flask; and (iv) expansion up to 2500 mL working volume in each of two 5 L Production Flasks (Rep/Cap Production Flask and Transgene Production Flask).

The Rep/Cap Production Flask was incubated until cell concentration was expanded to 1.8-2.5×10⁶ cells/mL and was then infected with Rep/Cap Source BIIC (Sf9:BIIC Infection Ratio of 1.0 ×10⁴ cell to cell (c/c), equivalent to 1.0×10⁵ (v/v) infection ratio). The infected cells were incubated for 72 hours (target cell diameter of ≥19.0 μm, cell culture density target of ≥3.0×10⁶ cells/mL), and then harvested by spinning down (polypropylene centrifuge tubes, 5 min at 4.0° C.) and resuspending the cell pellet at 2.0×10⁷ cells/mL in 50% 2× Freezing media (858 mL/L of ESF-AF Media, 140 mL/L of Dimethyl Sulfoxide, 113 mL/L of Trehalose, dihydrate) and 50% ESF-AF Media. The resuspended culture of Rep/Cap Infection BIICs was aliquoted into 2 mL or 5 mL ctyovials and stored in LN₂ vapor.

The Transgene Production Flask was incubated until cell concentration was expanded to 1.8-2.5×10⁶ cells/mL and was then infected with Transgene Source BIIC (Sf9:BIIC Infection Ratio of 1.0 ×10⁴ cell-to-cell (c/c), equivalent to 1.0×10⁵ v/v infection ratio). The infected cells were incubated for 96-100 hours (target cell diameter of ≥19.0 μm, cell culture density target of 3.0×10⁶ cells/mL), and then harvested by spinning down (polypropylene centrifuge tubes, 5 min at 4.0° C.) and resuspending the cell pellet at 2.0×10⁷ cells/mL in 50% 2× Freezing media (858 mL/L of ESF-AF Media, 140 mL/L of Dimethyl Sulfoxide, 113 mL/L of Trehalose, dihydrate) and 50% ESF-AF Media. The resuspended culture of Transgene Infection BIICs was aliquoted into 2 ml, or 5 ml, cryovials and stored in LN₂ vapor.

### Example 6. Upstream Production of Bulk Particle Pool (A)

One vial of the SN 9f4 CB was thawed in a 125 mL shaker flask (37° C. using waterbath, 1-5 minutes until ice crystals dissipate), and then diluted into 20 mL working volume of ESF-AF culture medium. The shaker flask was incubated at 27 CC (130-150 rpm shaking, 25 mm orbital diameter) for about 48 hours in a non-humidified, ambient air, temperature regulated incubator in a first expansion until the cell density was expanded to between 5.0-8.0×10⁶ cells/mL.

The culture was then seeded and expanded through multiple additional expansion steps using larger shaker flasks, with a target output density of 5.0×10⁶-1.0×10⁷ cells/mL for each expansion step to allow for a consistent target seeding density of 1.0-1.5×10⁶ cells/mL in subsequent expansion steps. Expansions were completed at 27° C. for 3-5 days with 130-150rpm shaking (≤400 mL working volume) 100-120 rpm shaking (>400 mL working volume).

The following additional expansions were completed: (i) expansion up to 100 mL working volume in a 250 or 500 mL flask; (ii) expansion up to 400 mL working volume in a 1.0 L flask; (iii) expansion up to 1500 mL working volume in a 3 L flask; and (iv) expansion up to 2500 mL working volume in each of two 5 L flasks (5000 mL total working volume).

The expanded culture mixture was transferred to a 50 L GE WAVE bioreactor (0.25 mL/min fixed air sparge, oxygen on demand up to 40% dissolved O₂, 20 rpm rocking, 9° rocking angle, 250 mL/min air inlet) for an additional expansion (3-5 days at 27° C.) up to a 25 L working volume with a target output density of 5.0-8.0×10⁶ cells/mL. The culture medium was then seeded into a stirred-tank GE Xcellerax Bioreactor (68 rpm agitation, 0.5mL/min fixed air sparge, cascading oxygen on demand up to 40% dissolved O₂, 0.5 mL/min headspace flow rate), and expanded (N-1 Bioreactor step, 2-3 days at 27° C.) up to a 125 L working volume with a target output density of 1.5-2.0×10⁶ cells/mL.

The culture mixture was seeded into a single-use Production Bioreactor with a seeding density of 0.8-1.5×10⁶ cells/int and 200 L working volume. The culture medium was further expanded in the Bioreactor (6 W/m³ impeller, 0.8 mL/min fixed air sparge, cascading oxygen on demand up to 40% dissolved O₂, 0.8 mL/min headspace flow rate) up to 3.0-3.2×10⁶ cells/mL in a 200 L working volume.

The cells in the Bioreactor were then co-infected with Rep/Cap Infection BIICs (1:250k v/v) and Transgene Infection BIICs (1:80k v/v). Infected cells were incubated for 144 hours (6 days) and the bulk harvest was collected for lysis and processing through Downstream processing.

Samples were taken through each of the expansion and bioreaction steps to monitor cell density and viability throughout the upstream process.

In one alternative, the expansion Bioreactor was a Pall 200 L Allegro Bioreactor maintained as 35 rpm agitation, 1.3 mL/min fixed air sparge, cascading oxygen on demand up to 40% dissolved O₂, and 0.8mL/min headspace flow rate.

In one alternative, the processing parameters for the Production Bioreactor were 41 agitation rpm (pre-infection), 51 agitation rpm (post-infection), 2.5 mL/min fixed air sparge, cascading oxygen on demand up to 40% dissolved O₂, and 1.2 mL/min headspace flow rate, with a target expansion up to 3.2-3.4×10⁶ cells/mt in a 200 L working volume.

In one alternative, the expanded culture mixture was transferred to a Pall Allegro XRS 25 L Bioreactor for an additional expansion (25 cpm agitation, cascading oxygen on demand up to 40% dissolved O₂, 0.3 mL/min fixed air sparge, 0.5 mL/min headspace flow rate 3-5 days at 27° C.) up to a 10 L working; volume with a target output density of 5.0×10⁶-1.0×10⁷ cells/mL.

In one alternative, the N-1 bioreactor was a Pall I25L Allegro Bioreactor (45 rpm agitation, cascading oxygen on demand up to 40% dissolved O₂, 0.8 L/min air overlay, 27° C. vessel temp, 1.5 L/min O₂ flow rate) with a target output density of 5.0×10⁶-1.0×10⁷ cells/mL.

In one alternative, the N production bioreactor was a PD 200L Allegro Bioreactor (60 rpm agitation, cascading oxygen on demand up to 40% dissolved O₂, 1.2 L/min air overlay, 27±1° C. vessel temp, 2.5 L/min O₂ flow rate). The culture mixture was seeded into the Production Bioreactor with a target seeding density of about 1.0×10⁶ cells/mL and 200 L working volume. The culture medium was further expanded in the Bioreactor up to about 3.2×10⁶ cells/ad, in a 200 L working volume. The cells in the Bioreactor were then co-infected with Rep/Cap Infection BIICs (1:300k v/v) and Transgene Infection BACs (1:100k v/v). Infected cells were incubated for 168 hours (7 days). Post-infection, the bioreactor conditions were adjusted as follows: 70 rpm agitation, cascading oxygen on demand up to 40% dissolved O₂, 1.2 L/min air overlay, 27° C. vessel temp, 3.0 L/min O₂ flow rate. The bulk harvest was collected for lysis and processing through Downstream processing.

### Example 7. Upstream—Production of Bulk Particle Pool (B)

One vial of the Sf9 9f4 CB was thawed in a 125 mL shaker flask (37° C. using waterbath, 1-5 minutes until ice crystals dissipate), and then diluted into 20 mL working volume of ESE-AF culture medium. The shaker flask was incubated at 27° C. (100 rpm shaking, 2-inch orbital diameter) in a non-humidified, ambient air, temperature regulated incubator in a first expansion until the cell density was expanded to between 5.0-8.0×10⁶ cells/mL.

The culture was then seeded and expanded through multiple additional expansion steps using larger shaker flasks, with a target output density of 4.0-5.0×10⁶ cells/mL for each expansion step to allow for a consistent seeding density of 0.5×10⁶ cells/mL in subsequent expansion steps. Expansions were completed at 27° C. for 3-5 days with 100 rpm shaking (≤2 L working volume) or 80 rpm shaking (>2 L working volume).

The following additional expansions were completed: (i) expansion up to 200 mL working volume in a 1 L flask; (ii) expansion up to 1000 mL working volume in a 3 L flask; and (iii) expansion up to 3000 mL working volume in 5 L flasks.

5 L of expanded culture mixture was spiked with 10% w/v Pluronic F-68 (2.045 v/v spike), which was then transferred to a 50 L GE WAVE bioreactor (0.25 mL/min fixed air sparge, oxygen on demand up to 40% dissolved O₂, 20 rpm rocking up to 9° angle) for an additional expansion (3-5 days at 27° C.) up to a 25 L working volume with a target output density of 3.0×10⁶ cells/mL.

The culture medium was spiked again with 10% w/v Pluronic F-68 (2.045 v/v spike) and then seeded into a GE 250 L Xcellerax Bioreactor with a seeding density of 0.8×10⁶ cells/mi. and 125 L working volume (Hvcione SFX Insect Cell Culture Media). The culture medium was expanded in the Bioreactor for 2-4 days (cascading oxygen on demand up to 40% dissolved O₂, 1 L/min air overlay, 27° C. vessel temp, 60° C. vent heater temp, downward mixer direction of 80 rpm) up to 3.0×10⁶ cells/mL in a 200 L working volume.

The cells in the Bioreactor were then co-infected with Rep/Cap Infection BIICs and Transgene Infection BIICs (1:1 BLIC ratio, 5.0 ×10³ SF9:BIIC ratio). Infected cells were incubated for 5-7 days and the bulk harvest was collected for lysis and processing through Downstream processing.

Samples were taken through each of the expansion and bioreaction steps to monitor cell density and viability throughout the upstream process.

### Example 8. Upstream—Production of Bulk Particle Pool (C)

One vial of the Sf9 9f4 CB was thawed in a 125 mL shaker flask (37° C. using waterbath, 1-5 minutes until ice crystals dissipate), and then diluted into 20 mL working volume of ESF-AF culture medium. The shaker flask was incubated at 27° C. (100 rpm shaking, 2-inch orbital diameter) in a non-humidified, ambient air, temperature regulated incubator in a first expansion until the cell density was expanded to between 5.0-8.0×10⁶ cells/mL.

The culture was then seeded and expanded through multiple additional expansion steps using larger shaker flasks, with a target output density of 4.0-5.0×10⁶ cells/mL for each expansion step to allow for a consistent seeding density of 0.5×10⁶ cells/mL in subsequent expansion steps. Expansions were completed at 27° C. for 3-5 days with 100 rpm shaking (≤2 L working volume) or 80 rpm shaking (>2 L working volume).

The following additional expansions were completed: (i) expansion up to 200 mL working volume in a 1 L flask; (ii) expansion up to 1000 mL working volume in a 3 L flask; and (iii) expansion up to 3000 mL working volume in 5 L flasks.

5 L of expanded culture mixture was spiked with 10% w/v Pluronic F-68 (2.045 v/v spike), which was then transferred to a 50 L GE WAVE bioreactor (0.25 mL/min fixed air sparge, oxygen on demand up to 40% dissolved O₂, 20 rpm rocking up to 9° angle) for an additional expansion (3-5 days at 27° C.) up to a 25 L working volume with a target output density of 3.0×10⁶ cells/mL.

The culture medium was spiked again with 10% w/v Pluronic F-68 (2.5 v/v spike) and then seeded into a Thermofisher 250 L HyPerfonna Bioreactor with a seeding density of 1.0-2.0×10⁶ cells/mL and 125 L working volume (Hyclone SFX Insect Cell Culture Media). The culture medium was expanded in the Bioreactor for 2-3 days (cascading oxygen on demand up to 40% dissolved O₂, 0.25 L/min air sparge, 7 L/min air overlay, 27° C. vessel temp, 65° C. vent heater temp, 60 rpm mixer) up to 2.5-2.75×10⁶ cells/mL in a 200 L working volume.

The cells in the Bioreactor were then co-infected with Rep/Cap Infection BIICs (1:250k v/v) and Transgene Infection BIICs (1:50k v/v). Infected cells were incubated for 1-2 days and the bulk harvest was collected for lysis and processing through Downstream processing.

Samples were taken through each of the expansion and bioreaction steps to monitor cell density and viability throughout the upstream process.

### Example 9. Downstream—Cell Lysis

Chemical Lysis was initiated on the bulk harvest in the Production Bioreactor by adding 0.2 M Arginine HCl (pH 7.5, 11.43% v/v), followed by 10% Triton X-100 surfactant (2.86% v/v, for 0.25% v/v Triton X-100 in final lysis mixture), followed by Benzonase nuclease (Grade I, 99% pure, 10 U/mL), and finally 2 M Tris Base to provide a lysis pH of 6.9-7.1. The lysis mixture was held at 37° C. for 4.0-6.0 hours at 6 W/m³ agitation, until a crude lysate pool was generated. The crude lysate pool was be brought to room temperature and aseptically sampled prior to further processing.

In one alternative, Chemical Lysis was initiated on the 200 L hulk harvest in the Production Bioreactor by adding 0.2 M Arginine HCl (pH 7.5, 11.43% v/v), followed by 10% Triton X-100 surfactant (5.3% v/v, for 0.5% w/v Triton X-100 in final lysis mixture) to provide a lysis pH of 6.8-7.5. The lysis mixture was held at 27° C. for 4.0-6.0 hours at 6 W/m³ agitation, until a crude lysate pool was generated.

In one alternative, chemical lysis was initiated on the bulk harvest in the Production Bioreactor (225 L working volume) by adding 2M Tris Base to provide a lysis pH of 6.9-7.1, followed by adding 0.2M Arginine HCl (pH 7.5, 30 kg of 1.18 kg/L), then Sartorius Denarase nuclease (5 U/mL), and finally 20% Triton X-100 surfactant in PBS background (3.25% kg of 1.00 kg/L). The lysis mixture was held at 37° C. for 3-4 hours at 60 rpm agitation, until a crude lysate pool was generated.

In one alternative, chemical lysis was initiated on the bulk harvest in the Production Bioreactor by adding 10% Triton X-100 surfactant (2.57% v/v spike), followed by Benzonase nuclease (Grade I, 99% pure, 10 U/mL). The lysis mixture was held at 37° C. for 6-12 hours with agitation, until a crude lysate pool was generated.

In one alternative, 0.5M Arginine or Arginine HCl is used in place of 0.2M Arginine HCl.

### Example 10. Downstream—Depth Filtration

A crude lysate pool from Chemical Lysis was processed through Depth Filtration using an EMD Millipore Millistak⁺ POD filter (DOFIC media series) (≤250 L/m² load challenge, 150-200 LMH load flux) with a differential pressure of less than 30 psid and an inlet pressure of less than 50 psig. A filter recovery flush using 20 mM sodium phosphate, 350 mM sodium chloride and 0.001% w/v Pluronic F-68 (mixture pH of 7.4) was passed through the depth filter, with the flushed recovery being added to the depth filtered pool.

In one alternative, the crude lysate, pool from Chemical Lysis was processed through Depth Filtration using an EMD Millipore Millistak⁺ POD filter (COSP media series) (≤250 L/m² load challenge).

In one alternative, the recovery flush (10-20 L/m² load challenge, 150-200 LMH load flux) used 50 mM sodium phosphate, 350 mM. sodium chloride and 0.001% w/v Pluronic F-68 (mixture pH of 7.4). In one alternative, the recovery flush (9-12 L/m² load challenge) used PBS.

### Example 11. Downstream—0.2 μm Filtration

A depth filtered pool from Depth Filtration was processed through 0.2 μm Filtration using an EMD Millipore Express SFIC XL10 0.5/0.2 μm filter (≤250 L/m² load challenge, 300 LMH load flux) with a differential pressure of less than 72.5 psid, an inlet pressure of less than 80 psig and a back pressure of less than 29 psig. A filter recovery flush using 20 mM sodium phosphate, 350 mM sodium chloride and 0.001% w/v Pluronic F-68 (mixture pH of 7.4) was passed through the 0.2 μm filter, with the flushed recovery being added to the 0.2 μm filtered pool. The resulting 0.2 μm filtered pool is spiked with 5 M NaCl (7.0-7.5% v/v) and held for 1-2 days to form a clarified lysate pool. The clarified lysate pool was stored at 2-8° C.

In one alternative, the 0.2 μm Filtration used a Sartorius Sartopore 2XLG, 0.8/0.2 lam filter with a 300 L/m² load challenge. In another alternative, the 0.2 μm Filtration comprised a recovery flush using 50 mM sodium phosphate, 350 mM sodium chloride and 0.001% w/v Pluronic F-68 (mixture pH of 7.4).

### Example 12. Downstream Affinity Chromatograohy

A clarified lysate pool from Depth Filtration and 0.2 μm Filtration was processed through Affinity Chromatography (AFC) using a GE AVB Sepharose HP column resin (3.0-3.5 L column volume). The column resin was equilibrated (3-7 column volumes (CV), 150 cm/hr) with a mixture of 20 mM sodium phosphate, 350 mM sodium chloride and 0.001% w/v Pluronic F-68 (mixture pH of 7.4′. The column resin was then loaded with the clarified lysate pool (≤5.0×10¹³ VG/mL-r load challenge) at 18-25° C., and then flushed (1-3 CV, 150 cm/hr) with a mixture of 20 mM sodium phosphate, 350 mM sodium chloride and 0.001% w/v Pluronic F-68 (mixture pH of 7.4). This was followed by a first wash of the column resin (5 CV, 150 cm/hr) with a mixture of 20 mM sodium citrate, 1M sodium chloride and 0.001% w/v Pluronic F-68 (mixture pH of 6.0); and a second wash of the column resin (5 CV, 150 cm/hr) with a mixture of 10 mM sodium citrate, 350 sodium chloride and 0.001% w/v Pluronic F-68 (mixture pH of 6.0). The filtered product was then eluted from the column resin (2-3 CV, 150 cm/hr) using a mixture of 20 mM sodium citrate, 350 mM sodium chloride and 0.001% w/v Pluronic F-68 (mixture pH of 3.0), with a target elution pool of 2.5-3.0 CV.

The resulting elution pool was neutralized with 0.5 M Tris Base and 0.001% w/v Pluronic F-68 (8-12% v/v, target final pH of 8.0). The neutralized elution pool was then processed through 0.2 μm Filtration using an EMI) Millipore Express SHC XL6000 0.5/0.2 filter (≤1000 L/m² load challenge, ≤30 psi, ≤400 L/m²/hr flow rate), resulting in an AFC pool (also referred to as an AVB pool) with a working pool volume of 8.5-9.0 L.

In one alternative, the clarified lysate pool was spiked with 5 M NaCl (7.53% v/v) before being processed through Affinity Chromatography.

In one alternative, the column resin was equilibrated (5-7 CV, 150 cm/hr) and flushed (2 CV, 150 cm/hr) with a mixture of 50 mM sodium phosphate, 350 mM sodium chloride and 0.001% w/v Pluronic F-68 (mixture pH of 7.4).

In one alternative, the column resin was not flushed before the first and second wash steps.

In one alternative, the resulting elution pool was neutralized with 2 M Tris Base and 0.001% wily Plutonic F-68 (3.0% v/v spike, target final pH of 8.0-8.5).

In one alternative, the clarified lysate pool was processed through AFC using Poros AAVX or Poros AAV9 resins, with glycine used for elution in place of citrate. In one alternative, the wash solution comprised 150 mM sodium chloride instead of 350 mM sodium chloride. In one alternative, the wash solution comprised 0 mM sodium chloride instead of 350 mM sodium chloride.

In one alternative, the clarified lysate pool was processed through AFC using Poros AAV9 resins (≤5.0×10¹³ VG/mL-r load challenge, 4-minute residence), with a first wash as disclosed above and a second wash which comprised a mixture of 50 mM sodium citrate, 350 mM sodium chloride and 0.001% w/v Pluronic F-68 (mixture pH of 5.5), followed by an elution mixture which comprised 200 mM glycine (pH 2).

In one alternative, glycine was used for elution in place of citrate. In one alternative, the wash solution comprised 150 mM sodium chloride instead of 350 mM sodium chloride. In one alternative, the wash solution comprised 0 mM sodium chloride instead of 350 mM sodium chloride.

In one alternative, the clarified lysate pool was processed through hydrophobic interaction chromatography (HIC) under the following conditions: Target Run Load of 5% bulk AAV drug product in 2M Ammonium Sulfate_{;} 0.001% F68, pH 7; Target Load Challenge of about 2.5-10.0×10¹² vg/mL-r; Equilibration with 2M Ammonium Sulfate, 50 mM Tris, 0.001% F68, pH 7.75; and Elution with 250 mM Ammonium Sulfate, 50 mM Tris, 0.001% F68, pH 7.75. in one alternative, the clarified lysate pool was processed through hydrophobic interaction chromatography (HIC) under the following conditions: Target Run Load of 5% bulk AAV drug product in 1M Ammonium Sulfate, 0.001% F68, pH 7; Target Load Challenge of about 2.5-10.0×10¹² vg/mL-r; Equilibration with 1M Ammonium Sulfate, 50 mM Tris, 0.001% F68, pH 7.75; and Elution with 250 mM Ammonium Sulfate, 50 mM Tris, 0.001% F68, pH 7.75. The HIC resins used were Tosoh PPG 600M, Tosoh Phenyl 650M, Tosoh Butyl 650M, or Toros Benzyl

### Example 13. Downstream—Ion-Exchange Chromatography

A neutralized AFC/AVB pool was processed through Anion-Exchange Chromatography (AEX) using a Sartorius Sartobind Q Membrane (150 mL membrane volume, bind-and-elute mode). The AEX membrane was equilibrated (20 membrane volumes (MV), 5-7 MV/min) with a first mixture of 20 mM Tris, 2 M sodium chloride and 0.001% w/v Pluronic F-68 (mixture pH of 8.0), and then a second mixture of 20 mM Tris, 100 mM sodium chloride and 0.001% w/v Plutonic F-68 (mixture pH of 8.0). The AFC pool was load adjusted by 1:4 dilution with 300-320% v/v of 20 mM Tris and 0.001% w/v Pluronic F-68 (mixture pH of 8.0), with a target pool conductivity of 10 mS/cm and a target pool pH of 8.0. The AEX membrane system was then loaded with the diluted AFC pool (4.0×10¹³ VG/mL-r load challenge) at 18-25° C. and 5 MV/min. The system was flushed (20 MV, 5 MV/min) with a mixture of 20 mM Tris, 100 mM sodium chloride and 0.001% w/v Pluronic F-68 (mixture pH of 8.0). The product was then eluted from the AEX membrane system (20 MV, 5 MV/min) with a mixture of 20 mM Tris, 220 mM sodium chloride and 0,001% w/v Pluronic F-68 (mixture pH of 8.0), with the entire elution being collected. The AEX elution pool was then processed through 0.2 μm Filtration using an EMD Millipore Express SHCXL150 filter (≤1000 L/m² load challenge, ≤30 psi), resulting in an AEX pool with a working pool volume of 1.5-2.0 L.

In one alternative, the neutralized AFC/AVB pool was processed through AEX using a Millipore Fractogel TMAE HiCap(m) Flow-Through membrane resin. The AEX membrane was charged and equilibrated (5 CV, 150 cm/hr) with a first mixture of 20 mM Tris, 2 M sodium chloride and 0.001% w/v Pluronic F-68 (mixture pH of 8.0), and then a second mixture of 40 mM Tris, 170 mM sodium chloride and 0.001% w/v Pluronic F-68 (mixture pH of 8.5). The AFC pool was load adjusted by 100-110% v/v spike of 10 mM Tris and 0.001% w/v Pluronic F-68 (mixture pH of 8.0-8.5), with a target pool conductivity of 17 mS/cm (adjusted with 5 M NaCl) and a target pool pH of 8.5 (adjust with 2 M Iris Base), The AEX membrane system was then loaded with the adjusted AFC pool (1.0-5.0×10¹³ VG/mL-r load challenge) at 18-25° C. The system was flushed and eluted (2 CV, 150 cm/hr) with a mixture of 40 mM Tris, 170 mM sodium chloride and 0.001% w/v Pluronic F-68 (mixture pH of 8.5), with the entire elution being collected. The AEX elution pool was then processed through 0.2 μm Filtration using an EMD Millipore Express SHCXL150 filter (≤1000 L/m² load challenge, ≤30 psi), resulting in an AEX pool.

In one alternative, the neutralized AFC/AVB pool was processed through AEX using a GE Q Sepharose HP membrane resin. The AEX membrane was equilibrated (5-20 MV, 150 cm/hr) with a mixture of 50 mM Bis-Tris Propane, 200 mM sodium chloride and 0.001%) w/v Pluronic F-68 (mixture pH of 9.0). The AFC pool was load adjusted up to a pH of 9.0 with 0.5 mM Bis-Tris Propane and 0.001% w/v Pluronic F-68, followed by 1:1 dilution with 100 mill Bis-Tris Propane, 20 mM sodium chloride and 0.001% w/v Pluronic F-68 (mixture pH of 9.0). The AEX membrane system was then loaded with the diluted AFC pool (≤1.0×10¹⁴ VG/mLr load challenge) at 18-25° C. and 150 cm/hr. The system was flushed and eluted (3 CV, 150 cm/hr) with a mixture of 50 mM Bis-Tris Propane, 200 mM sodium chloride and 0.001% w/v Pluronic F-68 (mixture pH of 9.0). The AEX elution pool was neutralized with a 10% v/v spike of 1 M Iris, 2 M NaCl, and 0.001% (w/v) Pluronic F-68 (mixture pH of pH 7.5). The AEX elution pool was then processed through 0.2 nm Filtration using an EMD Millipore Express SH CXL150 filter (≤1000 L/m² load challenge, ≤30 psi), resulting in an AEX pool.

In one alternative, the neutralized AFC/AVB pool was processed through AEX using a Sartorius Sartobind STIC Membrane (<2.5 L membrane volume per 250 L batch, bind-and-elute mode, ≤1.0×10¹⁴ VG/mLr load challenge). The system was flushed and eluted with a mixture of 20 mM sodium phosphate, 50 mM sodium chloride, and 0.001% (w/v) Pluronic F-68 (mixture pH of 6.8). In one alternative, the clarified lysate pool from Depth Filtration (Example 10) and 0.2 μm Filtration (Example 11) is processed through AEX prior to being processes through Affinity Chromatography (AFC) (Example 12).

In one alternative, the neutralized AFC/AVB pool was processed through Cation-Exchange Chromatography (CEX) using a Poros XS membrane resin. The CEX membrane was charged (6-10 CV, 500 cm/hr) with 1 M NaCl, then equilibrated with 20 mM Tris, 100 mM NaCl, and 0.001% (w/v) Pluronic F-68 (mixture pH of pH 8.5). The AFC pool was load adjusted up to a pH of 8.5 with 2 M Tris Base, followed by dilution with WFI water to target pool conductivity of 15 mS/cm, and then followed by the addition of 0.001% w/v of 1% Pluronic F-68. The CEX membrane system was then loaded with the diluted AFC pool (≤1.0×10¹⁴ VG/mL-r load challenge, 3.3-minute residence). The system was washed (6 CV, 3.3 minute residence) with 20 mM Tris and 0.001% (w/v) Pluronic F-68 (mixture pH of 8.5); followed by a first elution (10 CV, ≤500 cm/hr) with 20 mM Tris, 290 mM NaCl, and 0.001% (w/v) Pluronic F-68 (mixture pH of pH 8.5); and then a second elution (10 CV, ≤500 cm/hr) with 20 mM Tris, 305 mM NaCl, and 0.001% (w/v) Pluronic F-68 (mixture pH of pH 8.5), The CEX elution pool was neutralized with 1 M acetic acid to a mixture pH 7.0. The AEX elution pool was then processed through 0.2 p.m Filtration using an EMD Millipore Express SHC XL150 filter (50 L/m² load challenge), resulting in a CEX pool as an AEX pool equivalent.

In one alternative, the neutralized AFC/AVB pool was processed through AEX using a Poros HQ membrane resin (3.4 mL CV), with a target pool conductivity of 16-20 mS/cm at pH 7.8-8.2. In one alternative, the neutralized AFC/AVB pool was processed through AEX using the following parameters: Targeted Load Challenge of about 1×10¹³ vgs/ml-r, Target Load Conductivity of <5-6 mS/cm, Load pH of 8-10, Elution pH of 8-10 with Sodium Chloride and Sodium Acetate salts. In one alternative, the neutralized AFC/AVB pool was processed through Multimodal Chromatography (MMC) using a Nuvia aPrime 4A membrane using the following parameters: Targeted Load Challenge of about 1×10¹³ vgs/ml-r, Target Load Conductivity of <5-6 mS/cm, Load pH of 8-10, Elution pH of 8-10 with Sodium Chloride and Sodium Acetate salts. In one alternative, the neutralized AFC/AVB pool was processed through AEX or MMC using a pH of 9.5 or higher.

### Example 14. Downstream—TFF Filtration

A neutralized AEX pool was processed through Tangential Flow Filtration (TFF) using a Spectrum mPES Hollow Fiber TFF system (0.5 mm fiber ID, 100 kDA MWCO, 20-30 L/m² load challenge, 4-5 psi IMP, 2000 sec⁻¹ shear). The TFF system was first rinsed (20 L/m) with WFI water, then sanitized (20 L/m², 45 min hold) with 0.1 M NaOH, then equilibrated (20 L/m²) with an AEX Elution Buffer (pH 8.0) comprising 20 mM Tris, 220 mM sodium chloride, and 0.001% (w/v) Pluronic F-68, with equilibration continuing until both permeate and retentate effluents are at pH 8.0. The AEX pool was processed through Pre-TFF Nanofiltration using an Asahi Kasei Planova 35N filter as a single load, continuous flow process with constant pressure (12.5 L/m² load challenge, 10 psi) to produce a TFF Load pool. The TFF Load pool was spiked (11% v/v) with a 50% sucrose mixture, then processed through a first diafiltration (DF) step (5-6 diafiltration volumes (DV), low shear) using a first diafiltration buffer (high salt, low sucrose) which comprises 10 mM sodium phosphate, 1.5 mM potassium phosphate, 220 mM sodium chloride, 5% w/v Sucrose, and 0.001% (w/v) Poloxamer 188 (buffer pH of 7.5). Diafiltration of the product pool was followed by concentration through ultrafiltration to a target concentration of 7.0×10¹³ VG/mL (confirmed by ddPCR), and then a second diafiltration step (7-8 DV, low shear) using a final formulation buffer (low salt, high sucrose) which comprises 10 mM sodium phosphate (dibasic), 1.5 mM potassium phosphate (monobasic), 100 mM sodium chloride, 7% w/v Sucrose, and 0.001% (w/v) Poloxamer 188 (buffer pH of 7.5). Retentate comprising the product and final formulation buffer was collected into a Final TFF Pool. Viral titer of the Final TFF Pool was analyzed overnight using ddPCR.

The TFF system was subjected to a Recovery Flush (110% v/v of system holdup, 10 min recirculation) using final formulation buffer (low salt, high sucrose) which comprises 10 mM sodium phosphate (dibasic), 1.5 mM, potassium phosphate (monobasic), 100 mM sodium chloride, 7% w/v Sucrose, and 0.001% (w/v) Poloxamer 188 (buffer pH of 7.5). The Final TFF Recovery Flush is collected separately from the Final TFF Pool. Viral titer of the Final TFF Recovery Flush was analyzed overnight using ddPCR. Final TFF Recovery Flush was added to Final TFF Pool to provide VRF Load Pool with viral concentration of 3.5×10¹³ VG/mL. Additional final formulation buffer (low salt, high sucrose) was added as necessary to achieve target viral concentration for VRF Load Pool.

In one alternative, the neutralized AEX pool was processed through ITT using a Millipore Pellicon-3 Ultracef PLCTK system (30 kDA MWCO, 2-3 L/m² load challenge). The TFF system was equilibrated (20 L/m², 5-minute contact) with 20 mM Tris, 290 mM sodium chloride, and 0.001% (w/v) Pluronic F-68, with equilibration continuing until both permeate and retentate effluents are at pH 7.0. The TFF Load pool was diluted with 20 mM Iris, 290 mM. sodium chloride, and 0.001% (w/v) Pluronic F-68 to a viral concentration of 2.0-3.0×10¹² VG/mL. The TFF Load pool was not processed through Pre-TFF Nanofiltration, but was instead processed directly into a diafiltration step (6 DV, 5-10 psi TMP, 10-20 psi inlet, 80 LMH) using a diafiltration buffer which comprises 10 mM Sodium Phosphate, 180 mM Sodium Chloride, and 0.001% (w/v) Pluronic F-68 (mixture pH of 7.3). The pool was then concentrated through ultrafiltration (10-20 psi inlet, 80LMH) to a target concentration of 5.0×10¹² VG/mL. Retentate comprising the product and formulation buffer was collected into a Final TFF Pool. The process did not comprise a second diafiltration step or a Recovery Flush.

In one alternative, the neutralized AEX pool was processed through TFF using a Millipore Ultracel PLCTK system with Pellicon-3 cassette (0.57 in², 30 kDA MWCO, 2-3 L/m² load challenge, 6 psi TMP, 5-10 psi inlet feed). The TFF system was first rinsed (25 L/m²) with WFI water, then sanitized (25 L/m², 45 min hold) with 0.25 M NaOH, then equilibrated (25 L/m²) with an equilibration buffer (pH 8.5) comprising 40 mM Tris, 170 mM sodium chloride, and 0.001% (w/v) Pluronic F-68, with equilibration continuing until both permeate and retentate effluents are at pH 8.5, The IFF Load pool was not processed through Pre-TFF Nanofiltration or a first diafiltration step, but was instead concentrated through ultrafiltration (2.6 L/min) to a target concentration of 5.0×10¹² VG/mL (confirmed by ddPCR), and then a diafiltration step (8 DV, 2.6 L/min) using a diafiltration buffer which comprises 10 mM Sodium Phosphate, 180 mM Sodium Chloride, and 0.001% (w/v) Pluronic F-68 (mixture pH of 7.3). The TFF system was subjected to a Recovery Flush (110% v/v of system holdup, 5-10 min recirculation) using the same diafiltration buffer. The Final TFT Recovery Flush is collected separately from the Final TFT Pool, and each pool is processed separately through 0.2 μm Filtration using an EMD Millipore Express SHCXL150 filter (≤1000 L/m² load challenge, ≤400 L/m²/hr flow rate, ≤30 psi). Filtered TFF Recovery Flush was added to Filtered TFF Pool, and then diluted as needed with diafiltration buffer, to provide VRF Load Pool with viral concentration of 2.0-6.0×10¹² VG/mL.

In one alternative, the system uses Millipore Ultracel PLCTK system with Pellicon-3 cassette (0.57 m², 30 kDA MWCO, 2-3 L/m² load challenge, 25 psig at feed TMP, 350 LMH, 7 diafiltration volumes, <6.0×10¹⁶ VG/m² load).

In one alternative, the TFF system was equilibrated (10-15 L/m²) with a mixture of 10 mM sodium phosphate. 2 mM potassium phosphate, 2.7 mM potassium chloride, 192 mM sodium chloride, and 0.001% (w/v) Pluronic F-68 (mixture pH of 7.5). The TFF Load pool was not processed through Pre-TFF Nanofiltration or a first diafiltration step, but was instead concentrated through ultrafiltration (0.5 mm fiber ID, 100 kDA MWCO, 60 L/m² load challenge, 4-5 psi IMP, 4000 sec⁻¹ shear) to a target concentration of 5.0×10¹² VG/mL (confirmed by qPCR), and then a diafiltration step (5 DV, 4-5 psi TMP, 4000 sec⁻¹ shear) using a diafiltration buffer which comprises 10 mM Sodium Phosphate, 2 mM Potassium Phosphate, 2.7 mM Potassium Chloride, 192 mM Sodium Chloride, 0.001% (w/v) Pluronic F-68 (mixture pH of 7.5). The TFF system was subjected to a Recovery Flush (110% v/v of system holdup, 5 min recirculation) using a buffer which comprises 10 mM Sodium Phosphate, 2 mM Potassium Phosphate, 2.7 mM Potassium Chloride, 192 mM Sodium Chloride, and 0.001% (w/v) Pluronic F-68. Final TFF Recovery Flush was added to Final TFF Pool to provide VRF Load Pool with viral concentration of 3.0-5.0×1.0¹² VG/mL.

### Example 15. Downstream—Virus Retentive Filtration

A VRF Load Pool was processed through Virus Retentive Filtration (VRF) using art Asahi Kasei Planova 35N filter (50.0-100.0 L/m² load challenge, 10 psi) which had been processed through a pre-use flush (10.0 L/m² load challenge, 10 psi) with a formulation buffer of 10 mM sodium phosphate (dibasic), 1.5 mM potassium phosphate (monobasic), 100 mM sodium chloride, 7% w/v Sucrose, and 0.001% (w/v) Poloxamer 188 (buffer pH of 7.5). The VRF filtration was followed by processing through 0.2 μm Filtration using an EMD Millipore Express SHCXL150 filter (≤1000 L/m² load challenge, ≤30 psi), resulting in an VRF pool with a working viral concentration of 3.5-5.0×10¹² VG/mL.

The VRF pool was then processed through Millipore Final Filtration (FF) using an EMD Millipore Sterile Millipak 0.22 μm (Pre-use flush with formulation buffer, ≥10.0 L/m² load challenge; FF process with ≤1000 L/m² load challenge, 200 LMH load flux, ≤60 psi differential pressure, ≤75 psi inlet pressure) to provide a Drug Substance pool with a working viral concentration of 3.5-5.0×10¹² VG/mL. A portion of the Drug Substance pool was stored for ≤1 month at 2-8° C. in aseptic bioprocess bag closed to atmosphere. A portion of the Drug Substance pool was stored for ≥1 month at ≤−60° C. in aseptic Polypropylene container closed to atmosphere.

In one alternative, the VRF filter and FF filters are both pre-use-flushed (10 L/m², 240-300 LMH, 14 psi) with WFI water, followed by a second pre-use-flush with 10 sodium phosphate. 180 mM sodium chloride, and 0.001% Pluronic F68 (mixture pH of 7.3).

In one alternative, the VRF filter is pre-use-flushed (10-20 L/m²) with a mixture of 10 mM sodium phosphate, 2 mM Potassium Phosphate, 2.7 mM Potassium Chloride, 192 mM Sodium Chloride, and 0.001% Pluronic F68 (mixture pH of 7.5).

In one alternative, the VRF filter is a Millipore NFR filter.

### Example 16. Downstream—Fill and Finish

A Pooled Drug Substance was transferred to a Biosafety Cabinet (BSC) and filtered through a EMD Millipore Millipak Gamma Gold 0.22 μm filter (dual-in-line sterilizing grade filters, ≤1000 L/m² load challenge, 200 LMH load flux, ≤60 psi differential pressure, ≤75 psi inlet pressure). The filtered Drug Substance pool comprised 10 mM sodium phosphate, 180 mM sodium chloride, and 0.001% Pluronic F68 (mixture pH of 7.3), with a target AAV concentration of 3.0-5.0×10¹² VG/mL. The filtered Drug Substance pool was then aseptically filled into 2 ml Cryovials (1.8 ml fill volume, 1.6 ml extractable) utilizing a programmable Peristaltic dispensing pump within the BSC. Product vials were stoppered, seal capped, 100% visually inspected and labeled (at 25° C.), and then stored at ≤−65° C.

In one alternative, the Pooled Drug Substance was filtered through a Pall Supor EKV, 0.2 μm sterilizing-grade filter.

We claim:

1. A method for producing a recombinant adeno-associated virus (rAAV), comprising:
(a) introducing at least one viral production cell (VPC) into a bioreactor and expanding the number of VPCs in the bioreactor to a target VPC cell density;
(b) introducing into the bioreactor at least one expression baculovirus infected insect cell (BIIC) which comprises an AAV viral expression construct and at least one payload BIIC which comprises an AAV payload construct;
(c) incubating the mixture of VPCs, expression BIICs and payload BIICs in the bioreactor under conditions which result in the production of one or more rAAVs within one or more of the VPCs;
(d) harvesting a viral production pool from the bioreactor, wherein the viral production pool comprises a liquid media and the one or more VPCs containing the one or more rAAVs;
(e) exposing the one or more VPCs within the viral production pool to chemical lysis using a chemical lysis solution under chemical lysis conditions, wherein the chemical lysis releases the one or more rAAVs from the VPCs into the liquid media of the viral production pool;
(f) processing the viral production pool through one or more clarification filtration steps in which the viral production pool is processed through one or more clarification filtration systems;
(g) processing the viral production pool through one or more affinity chromatography steps in which the viral production pool is processed through one or more affinity chromatography systems;
(h) processing the viral production pool through one or more ion exchange chromatography steps in which the viral production pool is processed through one or more ion exchange chromatography systems;
(i) processing the viral production pool through one or more tangential flow filtration (TFF) steps in which the viral production pool is processed through one or more tangential flow filtration (TFF) systems; and
(j) processing the viral production pool through one or more virus retentive filtration (VRF) steps in which the viral production pool is processed through one or more virus retentive filtration (VRF) systems.

2. The method of claim 1, wherein the VPCs comprise Sf9 insect cells, and wherein the rAAVs are produced using a baculovirus production system.

3. The method of any one of claims 1-2, wherein the volume of the bioreactor is at least 5 L, 10 L, 20 L, 50 L, 100 L, or 200 L.

4. The method of any one of claims 1-3, wherein the target VPC cell density at BIIC Introduction is 2.0-4.0×10⁶ cells/mL, 2.5-3.5×10⁶ cells/mL, or about 3.0×10⁶ cells/mL.

5. The method of any one of claims 1-4, wherein the ratio of VPC cells at BIIC introduction relative to the number of expression BIICs introduced into the bioreactor is between 1:2.0×10⁵-1:4.0×10⁵ v/v, between 1:2.5×10⁵-1:3.5×10⁵ v/v, about 1:2.5×10⁵ v/v, about 1:3.0×10⁵ v/v, about 1:3.5×10⁵ v/v, or about 1:4.0×10⁵ v/v.

6. The method of any one of claims 1-5, wherein the ratio of VPC cells at BIIC introduction relative to the number of payload BIICs introduced into the bioreactor is between 1:5.0×10⁴-2.0×10⁵ v/v, between 1:8.0×10⁴-1:1.5×10⁵ v/v, about 1:8.0×10⁴ v/v, about 1:1.0×1.0⁵ v/v, or about 1:1.5×10⁵ v/v.

7. The method of any one of claims 1-6, wherein the ratio of expression BIICs introduced into the bioreactor relative payload BIICs introduced into the bioreactor is between 1:1-5:1, between 2:1-4:1, between 2.5:1-3.5:1, or about 3:1.

8. The method of any one of claims 1-7, wherein the one or more clarification filtration steps comprises processing the viral production pool through a depth filtration system, a 0.2 μm microfiltration system, or a combination thereof.

9. The method of any one of claims 1-7, wherein the one or more clarification filtration steps comprises processing the viral production pool through a depth filtration system and then a 0.2 μm microfiltration system.

10. The method of any one of claims 1-7, wherein the one or more clarification filtration steps comprises processing the viral production pool through a first depth filtration system, then a second depth filtration system, and then a 0.2 μm microfiltration system.

11. The method of any one of claims 1-10, wherein the one or more affinity chromatography steps comprises processing the viral production pool through one or more immunoaffinity chromatography systems in bind -elute mode; wherein the immunoaffinity chromatography system comprises one or more recombinant single-chain antibodies which are capable of binding to one or more AAV capsid variants.

12. The method of claim 11, wherein the affinity chromatography system comprises an AVB column resin, AAV9 column resin or AAVX column resin.

13. The method of any one of claims 1-12, wherein the one or more ion exchange chromatography steps comprises processing the viral production pool through one or more anion exchange chromatography systems in flow-through mode; wherein the anion exchange chromatography system comprises a stationary phase which binds non-viral impurities, non-AAV viral particles, or a combination thereof: and wherein the stationary phase of the anion exchange chromatography system does not hind to the one or more rAAVs in the viral production pool.

14. The method of claim 13, wherein the stationary phase of the anion exchange chromatography system comprises a quaternary amine functional group or a trimethylammonium ethyl (TMAE) functional group.

15. The method of any one of claims 1-14, wherein a 50% sucrose mixture is added to the viral production pool at a centration between 9-13% v/v prior to the one or more IFF steps.

16. The method of any one of claims 1-15, wherein the one or more TFF steps comprises a first diafiltration step in which at least a portion of the liquid media of the viral production pool is replaced with a low-sucrose diafiltration buffer, wherein the low-sucrose diafiltration buffer comprises between 4-6% w/v of a sugar or sugar substitute and between 150-250 mM of an alkali chloride salt, preferably between 4.5-5.5% w/v of sucrose and between 210-230 mM sodium chloride, and more preferably 5% w/v of sucrose and 220 mM sodium chloride.

17. The method of any one of claims 1-16, wherein the one or more TFF steps comprises an ultrafiltration concentration step, wherein the AAV particles in the viral production pool are concentrated to between 1.0×10¹²-5.0×10¹³ vg/mL, between 1.0-5.0×10¹³ vg/mL, between 2.0-3.0×10¹³ vg/mL, or about 2.7×10¹³ vg/mL.

18. The method of any one of claims 1-17, wherein the one or more TFF steps comprises a formulation diafiltration step in which at least a portion of the liquid media of the viral production pool is replaced with a high-sucrose formulation buffer, wherein the high-sucrose formulation buffer comprises between 6-8% w/v of a sugar or sugar substitute and between 90-100 mM of an alkali chloride salt, preferably 7% w/v of sucrose and between 90-100 mM sodium chloride, and more preferably 7% w/v of sucrose, 10 mM sodium phosphate, between 95-100 mM sodium chloride, and 0.001% w/v) Poloxamer 188.

19. The method of any one of claims 1-18, wherein the VRF system comprises a filter medium that retains particles that are 35 nm or larger, or a filter medium that retains particles that are 20 nm or larger.

20. A method of producing a pharmaceutical formulation, comprising. providing one or more rAAVs produced by the method of any one of claims 1-19; and (ii) combining the one or more rAAVs with one or more one pharmaceutical excipient.

21. A pharmaceutical formulation produced by the method of claim 20.

22. A method of producing a gene therapy product, comprising: (i) providing the pharmaceutical formulation of claim 21; and (ii) suitably aliquoting the pharmaceutical formulation into a formulation container.
